(19) United States
(12) Patent Application Publication
(10) Pub. No.: US 2003/0130485 A1
(43) Pub. Date: Jul. 10, 2003

(54) Novel human genes and methods of use thereof

(76) Inventors: Rachel E. Meyers, Newton, MA (US); Rory A. J. Curtis, Framingham, MA (US); Maria Alexandra Glucksmann, Lexington, MA (US); Rajasekhar Bandaru, Watertown, MA (US); Rosana Kapeller-Libermann, Chestnut Hill, MA (US)

Correspondence Address: LOUIS MYERS, Fish & Richardson P.C., 225 Franklin Street, Boston, MA, 02110-2804, US

(21) Appl. No.: 10/176,306

(22) Filed: Jun. 20, 2002

Related U.S. Application Data
(63) Continuation-in-part of application No. 10/001,137, filed on Nov. 14, 2001.
(63) Continuation-in-part of application No. PCT/US01/45291, filed on Nov. 14, 2001.
(60) Provisional application No. 60/248,362, filed on Nov. 14, 2000.
(60) Provisional application No. 60/248,331, filed on Nov. 14, 2000.
(60) Provisional application No. 60/248,365, filed on Nov. 14, 2000.
(60) Provisional application No. 60/250,077, filed on Nov. 30, 2000.
(60) Provisional application No. 60/250,327, filed on Nov. 30, 2000.
(60) Provisional application No. 60/250,176, filed on Nov. 30, 2000.

(30) Foreign Application Priority Data
Dec. 18, 2001 (WO) PCT/US01/49416
Oct. 22, 2001 (WO) PCT/US01/46717

(51) Int. Cl. C07K 14/435; C07K 16/40; C12P 21/02; C12N 5/06; C07H 21/04

(52) U.S. Cl. 530/350; 536/23.5; 530/388.1; 435/69.1; 435/320.1; 435/325

(57) ABSTRACT

The invention provides isolated nucleic acids molecules, designated 47476, 67210, 49875, 46842, 33201, 83378, 84233, 64708, 85041, 84234, 21617, 55562, 23566, 33489, and 57779 nucleic acid molecules, which encode novel human genes. The invention also provides antisense nucleic acid molecules, recombinant expression vectors containing 47476, 67210, 49875, 46842, 33201, 83378, 84233, 64708, 85041, 84234, 21617, 55562, 23566, 33489, or 57779 nucleic acid molecules, host cells into which the expression vectors have been introduced, and nonhuman transgenic animals in which a 47476, 67210, 49875, 46842, 33201, 83378, 84233, 64708, 85041, 84234, 21617, 55562, 23566, 33489, or 57779 gene has been introduced or disrupted. The invention still further provides isolated 47476, 67210, 49875, 46842, 33201, 83378, 84233, 64708, 85041, 84234, 21617, 55562, 23566, 33489, or 57779 proteins, fusion proteins, antigenic peptides and anti-47476, 67210, 49875, 46842, 33201, 83378, 84233, 64708, 85041, 84234, 21617, 55562, 23566, 33489, or 57779 antibodies. Diagnostic methods utilizing compositions of the invention are also provided.

### RELATED APPLICATIONS

This application is a continuation-in-part and claims priority to U.S. application Ser. No. 10/001,137, filed Nov. 14, 2001, and International Application Serial No. PCT/US01/45291, filed Nov. 14, 2001, which claim the benefit of U.S. Provisional Application Serial Nos. 60/248,362, 60/248,331, and 60/248,365, all filed on Nov. 14, 2000; and No. 60/250,077, 60/250,327, and 60/250,176, all filed on Nov. 30, 2000; and U.S. application Ser. No. 10/023,617, filed Dec. 18, 2001, and International Application Serial No. PCT/US01/49416, filed Dec. 18, 2001, which claim the benefit of U.S. Provisional Application Serial Nos. 60/256,249 and 60/256,405, both filed on Dec. 18, 2000; and U.S. application Ser. No. 10/083,248, filed Oct. 22, 2001, and International Application Serial No. PCT/US01/46717, filed Oct. 22, 2001, which claim the benefit of U.S. Provisional Application Serial Nos. 60/241,989, and 60/242,324, both filed on Oct. 20, 2000; and No. 60/242,518, filed on Oct. 23, 2000, the contents of which are incorporated herein by reference.

### BACKGROUND OF THE 47476, 67210, 49875, 46842, 33201, 83378, 84233, 64708, 85041, AND 84234 INVENTION

Guanine Nucleoside Dissociation Stimulators

Ras and ras-like proteins are membrane-associated molecular switches that bind and slowly hydrolyze GTP to GDP. Cell-surface receptors that signal through tyrosine kinases activate ras and/or ras-like proteins by stimulating a guanine nucleotide exchange reaction. In the case of ras, genetic and biochemical studies have indicated that this exchange reaction is controlled by the ras guanine nucleotide exchange factor Son of sevenless (SOS).

The balance between the GTP bound (active) and GDP bound (inactive) states of ras or a ras-like protein is regulated by the opposing action of proteins that activate the GTPase activity and proteins that promote the loss of bound GDP and the uptake of fresh GTP. The former proteins are GTPase-activating proteins, and the latter proteins are guanine nucleotide dissociation stimulators (GDSS) (or also as guanine nucleotide releasing (or exchange) factors (GRFs or GEFs)).

Proteins that act as GDSs can be classified into at least two families, CDC24 or CDC25, on the basis of sequence similarities. The size of the proteins in the CDC25 family range from 309 residues (LTE1) to 1596 residues (SOS). The sequence similarity shared by all GDS proteins is limited to a region of about 250 amino acids generally located in the C-terminal section (currently, the only exceptions are SOS and ralGDS, in which this domain makes up the central portion of the protein). This 250 amino acid domain has been shown to be essential for the activity of GDS proteins.

Glycosyltransferases

Glycosyltransferases catalyze the synthesis of glycoconjugates, including glycolipids, glycoproteins, and polysaccharides, by transferring an activated mono- or oligosaccharide residue to an existing acceptor molecule for the initiation or elongation of the carbohydrate chain. A catalytic reaction is believed to involve the recognition of both the donor and acceptor by suitable domains, as well as the catalytic site of the enzyme (Amado et al. (1999) Biochim Biophys Acta 1473:35-53; Kapitonov et al. (1999) Glycobiology 9:961-78). Evidence indicates that formation of glycosidic linkages is covered by large homologous glycosyltransferase gene families, and that the existence of multiple enzyme isoforms provides a degree of redundancy as well as a higher level of regulation (Kapitonov et al. (1999), supra).

Glycosylation is the principal chemical modification to proteins as they pass through Golgi vesicles. Glycosyltransferases of the Golgi do not possess an obvious sequence homology which would suggest a common Golgi retention signal. However, they are all membrane proteins and share type II topology, consisting of an amino terminal cytoplasmic tail, a signal anchor transmembrane domain, a stem region, and a large luminal catalytic domain. The membrane-spanning domain and its flanking regions contain necessary and sufficient information for Golgi retention of these enzymes (Jaskiewicz (1997) Acta Biochim Pol 44:173-9). ER localized glycosyltransferases can have either a type II topology, like the Golgi glycosyltransferases, or a type I topolgy, i.e., the N-terminus and catalytic domain reside in the lumen of the ER (Kapitonov et al. (1999), supra). Some glycosyltransferases are present on the cell surface and are thought to function as cell adhesion molecules by binding oligosaccharide substrates on adjacent cell surfaces or in the extracellular matrix. The best studied of these is beta 1,4-galactosyltransferase, which mediates sperm binding to the egg coat and selected cell interactions with the basal lamina (Shur (1993) Curr Opin Cell Biol 5:854-63).

DEAD Type Helicases

A large number of biological processes require the unwinding from double-stranded or base-paired regions of DNA/DNA, RNA/RNA or RNA/DNA hybrids to single-stranded polynucleotides. These complex reactions are dependent on helicases, mechanochemical enzymes that couple the energy of nucleoside triphosphate hydrolysis to the dehybridization or unwinding of duplex nucleic acid molecules. Helicases comprise a large number of proteins that share high sequence similarity (Aubourg et al. (1999) Nucleic Acids Res 27(2):628-36). A sequence based classification has led to the definition of three superfamilies of helicases, namely SF1, SF2 and SF3 (Gorbalenya and Koonin (1993) Curr Opin Struct Biol 3:419-429). To date, SF2 is the best characterized superfamily, which includes the DEAD and DEAH box (“DEAD/H”) helicases.

The DEAD box RNA helicase family has been defined by Linder et al. (1989), Nature 337:121-122, and named according to the highly conserved residues, Asp-Glu-Ala-Asp (D-E-A-D) motif. DEAD box RNA helicases differ mainly by the addition of N- and C-terminal sequences containing different targeting signals, RNA-binding motifs, and regions required for interactions with structural or regulatory proteins. Aubourg et al. (1999), supra.

Notwithstanding their shared nucleic acid unwinding activity, helicases are involved in a number of different molecular mechanisms, including viral replication, RNA splicing, ribosome assembly, and initiation of translation including transcription regulation (e.g., SNF2, STH1, brahma, MOT1), maintenance of chromosome stability during mitosis (e.g., lodestar), and various aspects of processing DNA damage, including DNA excision repair (e.g., RAD16 and ERCC6), recombinational pathways (e.g., RAD54) and post-replication daughter strand gap repair (e.g., RAD5) (Eisen et al. (1995) Nucleic Acid Res 23:2715-23; Schmid and Linder (1992) Mol Microbiol 6:283-292; and Stevenson et al. (1998) J Pathol 184:351-359). Thus, helicases are important, inter alia, in replication, regulation of transcription, and cellular growth and differentiation.

Centaurins

Centaurin proteins are a family of regulatory proteins that control the small GTPase Arf. One subclass of centaurins, the ccntaurin-g proteins, typically has at least three types of canonical protein domains: a plekstrin homology (PH) domain, an ArfGAP domain, and ankyrin domains (for a review, see Jackson et al. (2000) Trends. Biochem. Sci.25:489). Arf proteins are small guanine nucleotide binding proteins that regulate vesicular trafficking and the cytoskeleton. ArfGAP domains stimulate the intrinsic GTPase activity of Arf such that GTP bound to Arf is hydrolyzed to GDP. Centaurin proteins are implicated in the regulation of vesicle trafficking, and the actin cytoskeleton, such as in focal adhesions and membrane ruffling. As pivotal regulatory molecules in cell signalling networks, centaurins are potential modulators of cell motility, cell adhesion, and secretory events. Such physiological functions are commonly aberrant or perturbed in a number of diseases and pathologies.

Dehydrogenases/Reductases

The superfamily of dehydrogenases and reductases is comprised of numerous members, including dehydrogenase/reductases and quinone oxidoreductases. When members of the superfamily are aligned, only a few typically critical amino acid residues are conserved among the various proteins.

Humans possess nine variants of alcohol dehydrogenase/reductases (ADH). Dehydrogenase/reductases play fundamental roles in degradative, synthetic, and detoxification pathways. These enzymes catalyze the reversible oxidation of ethanol to acetaldehyde with the concomitant reduction of NAD. Some vertebrate ADHs metabolize alcohols other than ethanol, such as retinol (vitamin A). The ability of some ADHs to act as retinol dehydrogenases suggests that they may participate in the synthesis of retinoic acid, the active form of vitamin A involved in regulating cellular differentiation and embryonic development (Zgombic-Knoght et al. (1995) J. Biol. Chem. 270:10868-10877).

Dehydrogenase/reductases have been implicated in a variety of developmental processes and pathophysiological disease states. For example, allelic variations of ADH2 and ADH3 appear to influence the susceptibility to alcoholism and alcoholic liver cirrhosis in Asians (Thomasson et al. (1991) Am. J. Hum Genet. 48:677-681, Chao et al. (1994) Hepatology 19:360-366, and Higuchi et al. (1995) Am. J. Psychiatry 152:1219-1221). Furthermore, several lines of evidence indicate that first-pass metabolism of alcohol in humans may occur in the liver via the activity of members of the mammalian ADH family. First-pass metabolism is the difference between the quantity of ethanol that reaches the systemic circulation by the intravenous route and the quantity that reaches the system circulation by the oral route (Yin et al. (1999) Enzymology and Molecular Biology of Carbonyl Metabolism 7, Plenum Publishers, New York). A role for dehydrogenase/reductases has also been proposed in the etiology of Parkinson's disease (Buervenich et al. (2000) Mov. Disord. 15:813-818).

Quinone oxidoreductases catalyze the reduction of quinones. Members of this class of enzymes can contribute to the antitumor effects of certain bioreductive drugs by metabolizing anti-tumor quinones, thereby activating their cytotoxic effects (Fitzsimmons et al. (1996) J. Natl. Cancer Inst. 88:259-269).

Metal Transporters

The concentration of metallic ions such as cadmium, zinc, and cobalt is maintained within a narrow range in mammalian cells. A diverse family of metal ion transporter proteins, the “cation diffusion facilitator” family, contributes to the maintenance of cellular metallic ion homeostais (Paulsen et al. (1997) J. Membr. Biol. 156:99-103).

Zinc is an essential component of metalloenzymes, transcription factors, and other proteins, but can be toxic to mammalian cells at high concentrations. When intracellular zinc exceeds a certain concentration, it is thought to trigger regulatory cellular processes. Various homeostatic mechanisms are thought to be used by cells to regulate intracellular zinc: regulation of zinc influx across the plasma membrane; regulation of zinc efflux across the plasma membrane; sequestration of zinc within subcellular compartments; and synthesis of molecules, e.g., metallothioneins, that bind tightly to zinc (Palmiter et al. (1996) EMBO J. 15:1784-1791; Palmiter et al. (1996) Proc. Natl. Acad. Sci. USA 93:14934-14939).

The genes encoding several zinc transporters have been cloned. Each of the proteins encoded by these genes appears to contribute to cellular resistance to zinc toxicity. Zinc transporter-1 (ZnT-1) encodes a plasma membrane protein that stimulates zinc efflux. ZnT-1 appears to be activated by excess cellular zinc concentrations (Palmiter et al. (1995) EMBO J. 14:639-649). Zinc transporter-2 (ZnT-2) encodes a vesicular protein that promotes the vesicular sequestration of zinc. Thus, ZnT-2 appears to help protect cells from zinc toxicity by facilitating zinc transport into an endosomal/lysosomal compartment (Palmiter et al. (1996), EMBO J. 15:1784-1791). Zinc transporter-3 (ZnT-3) encodes a putative transporter of zinc into synaptic vesicles. ZnT-3, which is expressed in the brain and testis, is proposed to be a component of the complex that sequesters zinc in synaptic vesicles, thereby serving as a neuromodulator (Palmiter et al. (1996) Proc. Natl. Acad. Sci. USA 93:14934-14939). ZnT-1, ZnT-2, and ZnT-3 share a common topology characterized by six membrane-spanning domains, a histidine-rich cytoplasmic loop between membrane spanning regions four and five, and a long C-terminal tail.

Guanine nucleoside dissociation stimulators, glycosyltransferases, DEAD type helicases, centaurins, dehydrogenases/reductases, and metal transporters have all been implicated in human disease. Consequently, the isolation and characterization of additional guanine nucleoside dissociation stimulators, glycosyltransferases, DEAD type helicases, centaurins, dehydrogenases/reductases, and metal transporters will provide novel reagents for the treatment or prevention of disease, as well as new targets for the development of drugs that can be used to treat or prevent disease.

### SUMMARY OF THE 47476, 67210, 49875, 46842, 33201, 83378, 84233, 64708, 85041, AND 84234 INVENTION

The present invention is based, in part, on the discovery of novel Guanine Nucleotide Dissociation Stimulator, Glycosyltransferase, DEAD Type Helicase, Centaurin, Dehydrogenase/Reductase, and Metal Transporter family members, referred to herein as “47476, 67210, 49875, 46842, 33201, 83378, 84233, 64708, 85041, and 84234”. The nucleotide sequences of cDNAs encoding 47476, 67210, 49875, 46842, 33201, 83378, 84233, 64708, 85041, and 84234 are shown in SEQ ID NO:1, SEQ ID NO:4, SEQ ID NO:7, SEQ ID NO:10, SEQ ID NO:13, SEQ ID NO:16, SEQ ID NO:19, SEQ ID NO:22, SEQ ID NO:25, and SEQ ID NO:28, respectively, and the amino acid sequences of a 47476, 67210, 49875, 46842, 33201, 83378, 84233, 64708, 85041, and 84234 polypeptide is shown in SEQ ID NO:2, SEQ ID NO:5, SEQ ID NO:8, SEQ ID NO:11, SEQ ID NO:14, SEQ ID NO:17, SEQ ID NO:20, SEQ ID NO:23, SEQ ID NO:26, and SEQ ID NO:29, respectively. In addition, the nucleotide sequences of the coding regions are depicted in SEQ ID NO:3, SEQ ID NO:6, SEQ ID NO:9, SEQ ID NO:12, SEQ ID NO:15, SEQ ID NO:18, SEQ ID NO:21, SEQ ID NO:24, SEQ ID NO:27, and SEQ ID NO:30, respectively.

Accordingly, in one aspect, the invention features a nucleic acid molecule that encodes a 47476, 67210, 49875, 46842, 33201, 83378, 84233, 64708, 85041, or 84234 protein or polypeptide, e.g., a biologically active portion of the 47476, 67210, 49875, 46842, 33201, 83378, 84233, 64708, 85041, or 84234 protein. In a preferred embodiment the isolated nucleic acid molecule encodes a polypeptide having the amino acid sequence of SEQ ID NO:2, SEQ ID NO:5, SEQ ID NO:8, SEQ ID NO:11, SEQ ID NO:14, SEQ ID NO:17, SEQ ID NO:20, SEQ ID NO:23, SEQ ID NO:26, or SEQ ID NO:29. In other embodiments, the invention provides isolated 47476, 67210, 49875, 46842, 33201, 83378, 84233, 64708, 85041, or 84234 nucleic acid molecules having the nucleotide sequence shown in SEQ ID NO:1, SEQ ID NO:4, SEQ ID NO:7, SEQ ID NO:10, SEQ ID NO:13, SEQ ID NO:16, SEQ ID NO:19, SEQ ID NO:22, SEQ ID NO:25, or SEQ ID NO:28, having the nucleotide sequence shown in SEQ ID NO:3, SEQ ID NO:6, SEQ ID NO:9, SEQ ID NO:12, SEQ ID NO:15, SEQ ID NO:18, SEQ ID NO:21, SEQ ID NO:24, SEQ ID NO:27, or SEQ ID NO:30, or having the nucleotide sequence of the DNA insert of a plasmid deposited with an ATCC Accession Number as described herein. In still other embodiments, the invention provides nucleic acid molecules that are substantially identical (e.g., naturally occurring allelic variants) to the nucleotide sequence shown in SEQ ID NO:1, SEQ ID NO:4, SEQ ID NO:7, SEQ ID NO:10, SEQ ID NO:13, SEQ ID NO:16, SEQ ID NO:19, SEQ ID NO:22, SEQ ID NO:25, and SEQ ID NO:28, the nucleotide sequence shown in SEQ ID NO:3, SEQ ID NO:6, SEQ ID NO:9, SEQ ID NO:12, SEQ ID NO:15, SEQ ID NO:18, SEQ ID NO:21, SEQ ID NO:24, SEQ ID NO:27, or SEQ ID NO:30, or the nucleotide sequence of the DNA insert of a plasmid deposited with an ATCC Accession Number as described herein. In other embodiments, the invention provides a nucleic acid molecule which hybridizes under a stringency condition described herein to a nucleic acid molecule comprising the nucleotide sequence of SEQ ID NO:1, SEQ ID NO:4, SEQ ID NO:7, SEQ ID NO:10, SEQ ID NO:13, SEQ ID NO:16, SEQ ID NO:19, SEQ ID NO:22, SEQ ID NO:25, and SEQ ID NO:28, the nucleotide sequence of SEQ ID NO:3, SEQ ID NO:6, SEQ ID NO:9, SEQ ID NO:12, SEQ ID NO:15, SEQ ID NO:18, SEQ ID NO:21, SEQ ID NO:24, SEQ ID NO:27, or SEQ ID NO:30, or the nucleotide sequence of the DNA insert of a plasmid deposited with an ATCC Accession Number as described herein, wherein the nucleic acid encodes a full length 47476, 67210, 49875, 46842, 33201, 83378, 84233, 64708, 85041, or 84234 protein or an active fragment thereof.

In a related aspect, the invention further provides nucleic acid constructs that include a 47476, 67210, 49875, 46842, 33201, 83378, 84233, 64708, 85041, or 84234 nucleic acid molecule described herein. In certain embodiments, the nucleic acid molecules of the invention are operatively linked to native or heterologous regulatory sequences. Also included, are vectors and host cells containing the 47476, 67210, 49875, 46842, 33201, 83378, 84233, 64708, 85041, or 84234 nucleic acid molecules of the invention e.g., vectors and host cells suitable for producing 47476, 67210, 49875, 46842, 33201, 83378, 84233, 64708, 85041, or 84234 nucleic acid molecules and polypeptides.

In another related aspect, the invention provides nucleic acid fragments suitable as primers or hybridization probes for the detection of 47476, 67210, 49875, 46842, 33201, 83378, 84233, 64708, 85041, or 84234-encoding nucleic acids.

In still another related aspect, isolated nucleic acid molecules that are antisense to a 47476, 67210, 49875, 46842, 33201, 83378, 84233, 64708, 85041, or 84234 encoding nucleic acid molecule are provided.

In another aspect, the invention features, 47476, 67210, 49875, 46842, 33201, 83378, 84233, 64708, 85041, or 84234 polypeptides, and biologically active or antigenic fragments thereof that are useful, e.g., as reagents or targets in assays applicable to treatment and diagnosis of 47476, 67210, 49875, 46842, 33201, 83378, 84233, 64708, 85041, or 84234-mediated or -related disorders. In another embodiment, the invention provides 47476, 67210, 49875, 46842, 33201, 83378, 84233, 64708, 85041, or 84234 polypeptides having a 47476, 67210, 49875, 46842, 33201, 83378, 84233, 64708, 85041, or 84234 activity. Preferred polypeptides are 47476, 67210, 49875, 46842, 33201, 83378, 84233, 64708, 85041, or 84234 proteins including at least one Guanine Nucleotide Dissociation Stimulator, Glycosyltransferase, DEAD type helicase, Centaurin, Dehydrogenase/Reductase, or Metal Transporter domain, and, preferably, having a 47476, 67210, 49875, 46842, 33201, 83378, 84233, 64708, 85041, or 84234 activity, e.g., a 47476, 67210, 49875, 46842, 33201, 83378, 84233, 64708, 85041, or 84234 activity as described herein.

In other embodiments, the invention provides 47476, 67210, 49875, 46842, 33201, 83378, 84233, 64708, 85041, or 84234 polypeptides, e.g., a 47476, 67210, 49875, 46842, 33201, 83378, 84233, 64708, 85041, or 84234 polypeptide having the amino acid sequence shown in SEQ ID NO:2, SEQ ID NO:5, SEQ ID NO:8, SEQ ID NO:11, SEQ ID NO:14, SEQ ID NO:17, SEQ ID NO:20, SEQ ID NO:23, SEQ ID NO:26, OR SEQ ID NO:29 or the amino acid sequence encoded by the cDNA insert of the plasmid deposited with ATCC Accession Number as described herein; an amino acid sequence that is substantially identical to the amino acid sequence shown in SEQ ID NO:2, SEQ ID NO:5, SEQ ID NO:8, SEQ ID NO:11, SEQ ID NO:14, SEQ ID NO:17, SEQ ID NO:20, SEQ ID NO:23, SEQ ID NO:26, OR SEQ ID NO:29 or the amino acid sequence encoded by the cDNA insert of a plasmid deposited with an ATCC Accession Number as described herein; or an amino acid sequence encoded by a nucleic acid molecule having a nucleotide sequence which hybridizes under a stringency condition described herein to a nucleic acid molecule comprising the nucleotide sequence of SEQ ID NO:1, SEQ ID NO:4, SEQ ID NO:7, SEQ ID NO:10, SEQ ID NO:13, SEQ ID NO:16, SEQ ID NO:19, SEQ ID NO:22, SEQ ID NO:25, and SEQ ID NO:28, the nucleotide sequence of SEQ ID NO:3, SEQ ID NO:6, SEQ ID NO:9, SEQ ID NO:12, SEQ ID NO:15, SEQ ID NO:18, SEQ ID NO:21, SEQ ID NO:24, SEQ ID NO:27, or SEQ ID NO:30, or the nucleotide sequence of the DNA insert of a plasmid deposited with an ATCC Accession Number as described herein, wherein the nucleic acid encodes a full length 47476, 67210, 49875, 46842, 33201, 83378, 84233, 64708, 85041, or 84234 protein or an active fragment thereof.

In a related aspect, the invention provides 47476, 67210, 49875, 46842, 33201, 83378, 84233, 64708, 85041, or 84234 polypeptides or fragments operatively linked to non-47476, 67210, 49875, 46842, 33201, 83378, 84233, 64708, 85041, or 84234 polypeptides to form fusion proteins.

In another aspect, the invention features antibodies and antigen-binding fragments thereof, that react with, or more preferably specifically bind 47476, 67210, 49875, 46842, 33201, 83378, 84233, 64708, 85041, or 84234 polypeptides or fragments thereof, e.g., an extracellular domain or a catalytic domain of an 47476, 67210, 49875, 46842, 33201, 83378, 84233, 64708, 85041, or 84234 polypeptide.

In another aspect, the invention provides methods of screening for agents, e.g., compounds, that modulate the expression or activity of the 47476, 67210, 49875, 46842, 33201, 83378, 84233, 64708, 85041, or 84234 polypeptides or nucleic acids.

In still another aspect, the invention provides a process for modulating 47476, 67210, 49875, 46842, 33201, 83378, 84233, 64708, 85041, or 84234 polypeptide or nucleic acid expression or activity, e.g. using the screened compounds. In certain embodiments, the methods involve treatment of conditions related to aberrant activity or expression of the 47476, 67210, 49875, 46842, 33201, 83378, 84233, 64708, 85041, or 84234 polypeptides or nucleic acids, such as conditions involving aberrant or deficient cellular function resulting in a disorder, e.g., an immunological disorder, neurological disorder, metabolic disorder, cellular proliferation and/or differentiation disorder, disorder of metal ion imbalance, or a protein trafficing disorder.

In some embodiments, the agent, e.g., compound, is an inhibitor of a 47476, 67210, 49875, 46842, 33201, 83378, 84233, 64708, 85041, or 84234 polypeptide. Preferably, the inhibitor is chosen from a peptide, a phosphopeptide, a small organic molecule, a small inorganic molecule and an antibody (e.g., an antibody conjugated to a therapeutic moiety). In other embodiments, the compound is an inhibitor of a 47476, 67210, 49875, 46842, 33201, 83378, 84233, 64708, 85041, or 84234 nucleic acid, e.g., an antisense, a ribozyme, a double stranded RNA, or a triple helix molecule.

In some embodiments, the agent, e.g., compound, is an activator of a 47476, 67210, 49875, 46842, 33201, 83378, 84233, 64708, 85041, or 84234 polypeptide. Preferably, the activator is chosen from a peptide, a phosphopeptide, a small organic molecule, a small inorganic molecule and an antibody. In other embodiments, the compound activates (i.e., increases) the transcription or translation of a 47476, 67210, 49875, 46842, 33201, 83378, 84233, 64708, 85041, or 84234 nucleic acid, e.g., by directly or indirectly modulating the activity of a transcription or translation factor.

In some embodiments, the agent, e.g., compound is administered (e.g., to cells or to a subject) in combination with a second agent, e.g., a compound, e.g., a known therapeutic agent or compound. Such an agent, e.g., compound, could be used to treat or prevent immunological disorders, neurological disorders, metabolic disorders, cellular proliferation and/or differentiation disorders, disorders of metal ion imbalance, or a protein trafficing disorders.

In another aspect, the invention features methods for treating or preventing a disorder characterized by aberrant cellular proliferation or differentiation of a 47476, 67210, 49875, 46842, 33201, 83378, 84233, 64708, 85041, or 84234-expressing cell, in a subject. Preferably, the method includes administering to the subject (e.g., a mammal, e.g., a human) an effective amount of a compound (e.g., a compound identified using the methods described herein) that modulates the activity, or expression, of the 47476, 67210, 49875, 46842, 33201, 83378, 84233, 64708, 85041, or 84234 polypeptide or nucleic acid. In a preferred embodiment, the disorder is a cancerous or pre-cancerous condition.

In a further aspect, the invention provides methods for evaluating the efficacy of a treatment of a disorder, e.g., an immunological disorder, neurological disorder, metabolic disorder, cellular proliferation and/or differentiation disorder, disorder of metal ion imbalance, or a protein trafficing disorder. The method includes: treating a subject, e.g., a patient or an animal, with a protocol under evaluation (e.g., treating a subject with one or more of: chemotherapy, radiation, and/or a compound identified using the methods described herein); and evaluating the expression of a 47476, 67210, 49875, 46842, 33201, 83378, 84233, 64708, 85041, or 84234 nucleic acid or polypeptide before and after treatment. A change, e.g., a decrease or increase, in the level of a 47476, 67210, 49875, 46842, 33201, 83378, 84233, 64708, 85041, or 84234 nucleic acid (e.g., mRNA) or polypeptide after treatment, relative to the level of expression before treatment, is indicative of the efficacy of the treatment of the disorder. The level of 47476, 67210, 49875, 46842, 33201, 83378, 84233, 64708, 85041, or 84234 nucleic acid or polypeptide expression can be detected by any method described herein.

In a preferred embodiment, the evaluating step includes obtaining a sample (e.g., a tissue sample, e.g., a biopsy, or a fluid sample) from the subject, before and after treatment and comparing the level of expressing of a 47476, 67210, 49875, 46842, 33201, 83378, 84233, 64708, 85041, or 84234 nucleic acid (e.g., mRNA) or polypeptide before and after treatment.

In another aspect, the invention provides methods for evaluating the efficacy of a therapeutic or prophylactic agent (e.g., an anti-neoplastic agent). The method includes: contacting a sample with an agent (e.g., a compound identified using the methods described herein, a cytotoxic agent) and, evaluating the expression of 47476, 67210, 49875, 46842, 33201, 83378, 84233, 64708, 85041, or 84234 nucleic acid or polypeptide in the sample before and after the contacting step. A change, e.g., a decrease or increase, in the level of 47476, 67210, 49875, 46842, 33201, 83378, 84233, 64708, 85041, or 84234 nucleic acid (e.g., mRNA) or polypeptide in the sample obtained after the contacting step, relative to the level of expression in the sample before the contacting step, is indicative of the efficacy of the agent. The level of 47476, 67210, 49875, 46842, 33201, 83378, 84233, 64708, 85041, or 84234 nucleic acid or polypeptide expression can be detected by any method described herein. In a preferred embodiment, the sample includes cells obtained from the blood (e.g., hematopoietic cells, e.g., white blood cells or red blood cells), bone marrow, spleen, liver, kidney, stomach, a neural tissue, a cardiovascular tissue (e.g., heart, endothelial, or smooth muscle cells), or a cancerous tissue a cancerous tissue, e.g., a cancerous lung, breast, or ovary tissue.

The invention also provides assays for determining the activity of, or the presence or absence of, 47476, 67210, 49875, 46842, 33201, 83378, 84233, 64708, 85041, or 84234 polypeptides or nucleic acid molecules in a biological sample, including for disease diagnosis.

In further aspect, the invention provides assays for determining the presence or absence of a genetic alteration in a 47476, 67210, 49875, 46842, 33201, 83378, 84233, 64708, 85041, or 84234 polypeptide or nucleic acid molecule, including for disease diagnosis.

In another aspect, the invention features a two dimensional array having a plurality of addresses, each address of the plurality being positionally distinguishable from each other address of the plurality, and each address of the plurality having a unique capture probe, e.g., a nucleic acid or peptide sequence. At least one address of the plurality has a capture probe that recognizes a 47476, 67210, 49875, 46842, 33201, 83378, 84233, 64708, 85041, or 84234 molecule. In one embodiment, the capture probe is a nucleic acid, e.g., a probe complementary to a 47476, 67210, 49875, 46842, 33201, 83378, 84233, 64708, 85041, or 84234 nucleic acid sequence. In another embodiment, the capture probe is a polypeptide, e.g., an antibody specific for 47476, 67210, 49875, 46842, 33201, 83378, 84233, 64708, 85041, or 84234 polypeptides. Also featured is a method of analyzing a sample by contacting the sample to the aforementioned array and detecting binding of the sample to the array.

Other features and advantages of the invention will be apparent from the following detailed description, and from the claims.

### BRIEF DESCRIPTION OF THE DRAWINGS

FIG. 1 depicts a hydropathy plot of human 47476. Relative hydrophobic residues are shown above the dashed horizontal line, and relative hydrophilic residues are below the dashed horizontal line. Numbers corresponding to positions in the amino acid sequence of human 47476 are indicated. Polypeptides of the invention include fragments which include: all or part of a hydrophobic sequence, i.e., a sequence above the dashed line, e.g., the sequence from about amino acid 82 to 105, from about 341 to 360, and from about 521 to 540 of SEQ ID NO:2; all or part of a hydrophilic sequence, i.e., a sequence below the dashed line, e.g., the sequence of from about amino acid 106 to 122, from about 325 to 340, and from about 500 to 520 of SEQ ID NO:2.

FIGS. 2A-2C depict alignments of the ras guanine nucleotide dissociation stimulator domain of human 47476 with consensus amino acid sequences derived from a hidden Markov model (HMM) from PFAM and SMART. The upper sequence of FIG. 2A is the consensus amino acid sequence according to PFAM (SEQ ID NO:31), while the lower amino acid sequence corresponds to amino acids 195 to 381 of SEQ ID NO:2. FIG. 2B shows an alignment of the ras guanine nucleotide dissociation stimulator domain consensus amino acid sequence according to SMART (SEQ ID NO:32) with amino acids 197 to 433 of SEQ ID NO:2, while FIG. 2C shows an alignment of the guanine nucleotide dissociation stimulator N-terminal motif consensus sequence according to SMART (SEQ ID NO:33) with amino acids 55 to 172 of SEQ ID NO:2.

FIGS. 3A-3B depict alignments of the EF-hand calcium-binding domain of human 47476 with consensus amino acid sequences derived from a hidden Markov model (HMM) from PFAM and SMART, respectively. The upper sequence of FIG. 3A is the consensus amino acid sequence according to PFAM (SEQ ID NO:34), while the lower amino acid sequence corresponds to amino acids 470 to 498 of SEQ ID NO:2. FIG. 3B shows an alignment of the consensus amino acid sequence according to SMART (SEQ ID NO:35) with amino acids 470 to 498 of SEQ ID NO:2.

FIGS. 4A-4B depict alignments of the phorbol ester/diacylglycerol binding domain (C1 domain) of human 47476 with consensus amino acid sequences derived from a hidden Markov model (HMM) from PFAM and SMART. The upper sequence of FIGS. 4A and 4B are the consensus amino acid sequences according to PFAM (SEQ ID NO:36) and SMART (SEQ ID NO:37), respectively, while the lower amino acid sequences correspond to amino acids 541 to 590 of SEQ ID NO:2.

FIG. 5 depicts a hydropathy plot of human 67210. Relative hydrophobic residues are shown above the dashed horizontal line, and relative hydrophilic residues are below the dashed horizontal line. Numbers corresponding to positions in the amino acid sequence of human 67210 are indicated. Polypeptides of the invention include fragments which include: all or part of a hydrophobic sequence, i.e., a sequence above the dashed line, e.g., the sequence from about amino acid 10 to 25 of SEQ ID NO:5; all or part of a hydrophilic sequence, i.e., a sequence below the dashed line, e.g., the sequence of from about amino acid 240-260 of SEQ ID NO:5; a sequence which includes a Cys, or a glycosylation site

FIG. 6 depicts an alignment of the glycosyltransferase domain of human 67210 with a consensus amino acid sequence derived from a hidden Markov model (HMM) from PFAM. The upper sequence is the consensus amino acid sequence (SEQ ID NO:38), while the lower amino acid sequence corresponds to amino acids 63 to 340 of SEQ ID NO:5.

FIG. 7 depicts a hydropathy plot of human 49875. Relative hydrophobic residues are shown above the dashed horizontal line, and relative hydrophilic residues are below the dashed horizontal line. Numbers corresponding to positions in the amino acid sequence of human 49875 are indicated. Polypeptides of the invention include fragments which include: all or part of a hydrophobic sequence, i.e., a sequence above the dashed line, e.g., the sequence from about amino acid 285 to 295 of SEQ ID NO:8; all or part of a hydrophilic sequence, i.e., a sequence below the dashed line, e.g., the sequence of from about amino acid 520 to 550 of SEQ ID NO:8; a sequence which includes a Cys, or a glycosylation site.

FIGS. 8A-8B depict alignments of the DEAD-type helicase domain of human 49875 with consensus amino acid sequences derived from a hidden Markov model (HMM) from PFAM (PF00270) (FIG. 8A) and from SMART (FIG. 8B). The upper sequences are the consensus amino acid sequences according to PFAM (SEQ ID NO:39) and according to SMART (SEQ ID NO:40), while the lower amino acid sequences corresponds to amino acids 22 to 245 (FIG. 8A) or amino acids 28 to 245 (FIG. 8B) of SEQ ID NO:8.

FIGS. 9A-9B depict alignments of the conserved helicase C-terminal domain of human 49875 with consensus amino acid sequences derived from a hidden Markov model (HMM) from PFAM (PF00271) (FIG. 9A) and from SMART (FIG. 9B). The upper sequences are the consensus amino acid sequences according to PFAM (SEQ ID NO:41) and according to SMART (SEQ ID NO:42), while the lower amino acid sequences corresponds to amino acids 281 to 363 (FIG. 9A) or amino acids 281 to 363 (FIG. 9B) of SEQ ID NO:8.

FIG. 10 depicts a hydropathy plot of human 46842. Relative hydrophobic residues are shown above the dashed horizontal line, and relative hydrophilic residues are below the dashed horizontal line. The cysteine residues (cys) are indicated by short vertical lines just below the hydropathy trace. The numbers corresponding to the amino acid sequence of human 46842 are indicated. Polypeptides of the invention include fragments which include: all or part of a hydrophobic sequence, i.e., a sequence above the dashed line, e.g., the sequence from about amino acid 431 to 439, from about 558 to 566, and from about 706 to 719 of SEQ ID NO:11; all or part of a hydrophilic sequence, i.e., a sequence below the dashed line, e.g., the sequence of from about amino acid 567 to 580, from about 720 to 737, and from about 757 to 771 of SEQ ID NO:11; a sequence which includes a Cys, or a glycosylation site.

FIG. 11 depicts an alignment of the PH domain of human 46842 and the corresponding consensus amino acid sequences derived from a hidden Markov model (HMM) from PFAM. The upper sequence is the consensus amino acid sequence (SEQ ID NO:43), while the lower amino acid sequence corresponds to amino acids 269 to 363 of SEQ ID NO:11.

FIG. 12 depicts an alignment of the ArfGAP domain of human 46842 and the corresponding consensus amino acid sequences derived from a hidden Markov model (HMM) from PFAM. The upper sequence is the consensus amino acid sequence (SEQ ID NO:44), while the lower amino acid sequence corresponds to amino acids 403 to 525 of SEQ ID NO:11.

FIGS. 13A-13B depict alignments of the ankyrin domains of human 46842 and the corresponding consensus amino acid sequences derived from a hidden Markov model (HMM) from PFAM. The upper sequence is the consensus amino acid sequence (SEQ ID NO:45), while the lower amino acid sequence corresponds to amino acids 702 to 734 (FIG. 13A) and 735 to 767 (FIG. 13B) of SEQ ID NO:11.

FIG. 14 depicts a hydropathy plot of human 33201. Relative hydrophobic residues are shown above the dashed horizontal line, and relative hydrophilic residues are below the dashed horizontal line. Numbers corresponding to positions in the amino acid sequence of human 33201 are indicated. Polypeptides of the invention include fragments which include: all or part of a hydrophobic sequence, i.e., a sequence above the dashed line, e.g., the sequence from about amino acid 70 to 80, and from about 158 to 178 of SEQ ID NO:2; all or part of a hydrophilic sequence, i.e., a sequence below the dashed line, e.g., the sequence of from about amino acid 50 to 60, from about 82 to 90, and from about 205 to 210 of SEQ ID NO:14; a sequence which includes at least one Cys residue, or a glycosylation site.

FIGS. 15A-15B depicts an alignment of the dehydrogenase/reductase domain of human 33201 with a consensus amino acid sequence derived from a hidden Markov model (HMM) from PFAM. The upper sequence is the consensus amino acid sequence (SEQ ID NO:46), while the lower amino acid sequence corresponds to amino acids 22 to 345 of SEQ ID NO:14.

FIG. 16 depicts a hydropathy plot of human 83378. Relative hydrophobic residues are shown above the dashed horizontal line, and relative hydrophilic residues are below the dashed horizontal line. Numbers corresponding to positions in the amino acid sequence of human 83378 are indicated. Polypeptides of the invention include fragments which include: all or part of a hydrophobic sequence, i.e., a sequence above the dashed line, e.g., the sequence from about amino acid 325 to 335, from about 340 to 350, and from about 415 to 430 of SEQ ID NO:17; all or part of a hydrophilic sequence, i.e., a sequence below the dashed line, e.g., the sequence of from about amino acid 150 to 160, from about 220 to 235, and from about 355 to 370 of SEQ ID NO:17.

FIGS. 17A-17B depict alignments of the first and second cation efflux domains of human 83378 with consensus amino acid sequences derived from hidden Markov models (HMM) from PFAM. In FIG. 17A, the upper sequence is the consensus amino acid sequence (SEQ ID NO:47), while the lower amino acid sequence corresponds to the first cation efflux domain of human 83378 (amino acids 11 to 133 of SEQ ID NO:17). Similarly, in FIG. 17B, the upper sequence is the consensus amino acid sequence (SEQ ID NO:48), while the lower amino acid sequence corresponds to the second cation efflux domain of human 88378 (amino acids 231 to 389 of SEQ ID NO:17).

FIGS. 18A-8B depicts an alignment of the amino acid sequence of human 83378 (upper sequence; SEQ ID NO:17) with the amino acid sequence of rat ZnT-1 (lower sequence; GenBank3 Accession Number Q62720; SEQ ID NO:49).

FIGS. 18C-18D depicts an alignment of the amino acid sequence of human 83378 (lower sequence; SEQ ID NO:17) with the amino acid sequence of human GenBank3 Accession Number AL359609 (upper sequence; SEQ ID NO:50).

FIG. 19 depicts a hydropathy plot of human 84233. Relative hydrophobic residues are shown above the dashed horizontal line, and relative hydrophilic residues are below the dashed horizontal line. Numbers corresponding to positions in the amino acid sequence of human 84233 are indicated. Polypeptides of the invention include fragments which include: all or part of a hydrophobic sequence, i.e., a sequence above the dashed line, e.g., the sequence from about amino acid 220 to 230, from about 240 to 260, and from about 262 to 273 of SEQ ID NO:20; all or part of a hydrophilic sequence, i.e., a sequence below the dashed line, e.g., the sequence of from about amino acid 1 to 20, from about amino acid 80 to 90, and from about 150 to 160 of SEQ ID NO:20.

FIG. 20 depicts an alignment of the cation efflux domain of human 84233 with a consensus amino acid sequence derived from a hidden Markov model (HMM) from PFAM. The upper sequence is the consensus amino acid sequence (SEQ ID NO:51), while the lower amino acid sequence corresponds to amino acids 25 to 310 of SEQ ID NO:20.

FIG. 21 depicts an alignment of the amino acid sequence of human 84233 (lower sequence; SEQ ID NO:20) with the amino acid sequence of human ZnT-3 (upper sequence; GenBank3 Accession Number Q99726; SEQ ID NO:52).

FIG. 22 depicts a hydropathy plot of human 64708. Relative hydrophobic residues are shown above the dashed horizontal line, and relative hydrophilic residues are below the dashed horizontal line. Numbers corresponding to positions in the amino acid sequence of human 64708 are indicated. Polypeptides of the invention include fragments which include: all or part of a hydrophobic sequence, i.e., a sequence above the dashed line, e.g., the sequence from about amino acid 180 to 195, from about 290 to 300, and from about 340 to 350 of SEQ ID NO:23; all or part of a hydrophilic sequence, i.e., a sequence below the dashed line, e.g., the sequence of from about amino acid 123 to 133, from about 380 to 395, and from about 450 to 461 of SEQ ID NO:23.

FIGS. 23A-23B depict alignments of the first and second cation efflux domains of human 64708 with consensus amino acid sequences derived from hidden Markov models (HMM) from PFAM. In FIG. 23A, the upper sequence is the consensus amino acid sequence (SEQ ID NO:53), while the lower amino acid sequence corresponds to the first cation efflux domain of human 64708 (amino acids 55 to 153 of SEQ ID NO:23). Similarly, in FIG. 23B, the upper sequence is the consensus amino acid sequence (SEQ ID NO:54), while the lower amino acid sequence corresponds to the second cation efflux domain of human 64708 (amino acids 227 to 320 of SEQ ID NO:23).

FIGS. 24A-24B depicts an alignment of the amino acid sequence of human 64708 (upper sequence; SEQ ID NO:23) with the amino acid sequence of murine ZnTl1 (lower sequence; GenBank3 Accession Number AF233321; SEQ ID NO:55).

FIG. 24C depicts an alignment of the amino acid sequence of human 64708 (lower sequence; SEQ ID NO:23) with the amino acid sequence of human GenBank3 Accession Number AK000844 (upper sequence; SEQ ID NO:56).

FIG. 25 depicts a hydropathy plot of human 85041. Relative hydrophobic residues are shown above the dashed horizontal line, and relative hydrophilic residues are below the dashed horizontal line. Numbers corresponding to positions in the amino acid sequence of human 85041 are indicated. Polypeptides of the invention include fragments which include: all or part of a hydrophobic sequence, i.e., a sequence above the dashed line, e.g., the sequence from about amino acid 35 to 50, from about 440 to 470, and from about 685 to 695 of SEQ ID NO:26; all or part of a hydrophilic sequence, i.e., a sequence below the dashed line, e.g., the sequence of from about amino acid 320 to 340, from about 555 to 575, and from about 750 to 765 of SEQ ID NO:26.

FIGS. 26A-26B depicts an alignment of the cation efflux domain of human 85041 with a consensus amino acid sequence derived from a hidden Markov model (HMM) from PFAM. The upper sequence is the consensus amino acid sequence (SEQ ID NO:57), while the lower amino acid sequence corresponds to amino acids 419 to 733 of SEQ ID NO:26.

FIGS. 27A-27B depicts an alignment of the amino acid sequence of human 85041 (upper sequence; SEQ ID NO:26) with the amino acid sequence of murine ZnTl1 (lower sequence; GenBank3 Accession Number AF233321; SEQ ID NO:55).

FIG. 28 depicts a hydropathy plot of human 84234. Relative hydrophobic residues are shown above the dashed horizontal line, and relative hydrophilic residues are below the dashed horizontal line. Numbers corresponding to positions in the amino acid sequence of human 84234 are indicated. Polypeptides of the invention include fragments which include: all or part of a hydrophobic sequence, i.e., a sequence above the dashed line, e.g., the sequence from about amino acid 59 to 70, from about 330 to 345, and from about 370 to 376 of SEQ ID NO:29; all or part of a hydrophilic sequence, i.e., a sequence below the dashed line, e.g., the sequence of from about amino acid 10 to 20, from about 165 to 175, and from about 190 to 230 of SEQ ID NO:29.

FIG. 29 depicts an alignment of the cation efflux domain of human 84234 with a consensus amino acid sequence derived from a hidden Markov model (HMM) from PFAM. The upper sequence is the consensus amino acid sequence (SEQ ID NO:58), while the lower amino acid sequence corresponds to amino acids 38 to 349 of SEQ ID NO:29.

FIG. 30 depicts an alignment of the amino acid sequence of human 84234 (upper sequence; SEQ ID NO:29) with the amino acid sequence of murine ZnTl2 (lower sequence; GenBank™ Accession Number AF233322; SEQ ID NO:59).

FIG. 31 depicts a hydropathy plot of human 21617. Relative hydrophobic residues are shown above the dashed horizontal line, and relative hydrophilic residues are below the dashed horizontal line. Numbers corresponding to positions in the amino acid sequence of human 21617 are indicated. Polypeptides of the invention include fragments which include: all or part of a hydrophobic sequence, i.e., a sequence above the dashed line, e.g., the sequence from about 1 to 20, from about 191 to 203, and from about 293 to 310 of SEQ ID NO:64; all or part of a hydrophilic sequence, i.e., a sequence below the dashed line, e.g., the sequence of from about 68 to 77, from about 222 to 236, and from about 325 to 340 of SEQ ID NO:64.

FIG. 32 depicts an alignment of the short chain dehydrogenase domain of human 21617 with a consensus amino acid sequence derived from a hidden Markov model (HMM) from PFAM. The upper sequence is the consensus amino acid sequence (SEQ ID NO:69), while the lower amino acid sequence corresponds to amino acids 37 to 249 of SEQ ID NO:64.

FIG. 33 depicts a hydropathy plot of human 55562. Relative hydrophobic residues are shown above the dashed horizontal line, and relative hydrophilic residues are below the dashed horizontal line. Numbers corresponding to positions in the amino acid sequence of human 55562 are indicated. Polypeptides of the invention include fragments which include: all or part of a hydrophobic sequence, i.e., a sequence above the dashed line, e.g., the sequence from about amino acid 39 to 44, from about 66 to 76, and from about 156 to 167 of SEQ ID NO:67; and all or part of a hydrophilic sequence, i.e., a sequence below the dashed line, e.g., the sequence of from about amino acid 2 to 9, from about 95 to 110, and from about 259 to 273 of SEQ ID NO:67.

FIG. 34 depicts an alignment of the tetratricopeptide repeat domain of human 55562 with a consensus amino acid sequence derived from a hidden Markov model (HMM) from PFAM. The upper sequence is the consensus amino acid sequence (SEQ ID NO:70), while the lower amino acid sequence corresponds to amino acids 40 to 73 of SEQ ID NO:67.

FIG. 35 depicts a BLAST alignment of a portion of human 55562 that includes the tetratricopeptide domain with a consensus amino acid sequence derived from a ProDomain, PD314595 (Release 2001.1). The upper sequence is the consensus amino acid sequence (SEQ ID NO:71), while the lower amino acid sequence corresponds to human 55562, about amino acids 40 to 266 of SEQ ID NO:67.

FIG. 36 depicts a BLAST alignment of a portion of human 55562 that includes the tetratricopeptide domain with a consensus amino acid sequence derived from a ProDomain, PD014461 (Release 1999.2). The upper sequence is the consensus amino acid sequence (SEQ ID NO:72), while the lower amino acid sequence corresponds to human 55562, about amino acids 40 to 97 of SEQ ID NO:67.

FIG. 37 depicts a hydropathy plot of human 23566. Relative hydrophobic residues are shown above the dashed horizontal line, and relative hydrophilic residues are below the dashed horizontal line. Numbers corresponding to positions in the amino acid sequence of human 23566 are indicated. Polypeptides of the invention include fragments which include: all or part of a hydrophobic sequence, i.e., a sequence above the dashed line, e.g., the sequence from about amino acid 41 to 68, from about 130 to 150, and from about 270 to 285 of SEQ ID NO:74; all or part of a hydrophilic sequence, i.e., a sequence below the dashed line, e.g., the sequence of from about amino acid 70 to 100, from about 170 to 200, and from about 285 to 310 of SEQ ID NO:74; a sequence which includes a Cys, or a glycosylation site.

FIG. 38 depicts an alignment of the zinc carboxypeptidase domain of human 23566 with a consensus amino acid sequence derived from a hidden Markov model (HMM) from PFAM. The upper sequence is the consensus amino acid sequence (SEQ ID NO:82), while the lower amino acid sequence corresponds to amino acids 180 to 443 of SEQ ID NO:74.

FIG. 39 depicts a hydropathy plot of human 33489. Relative hydrophobic residues are shown above the dashed horizontal line, and relative hydrophilic residues are below the dashed horizontal line. Numbers corresponding to positions in the amino acid sequence of human 33489 are indicated. Polypeptides of the invention include fragments which include: all or part of a hydrophobic sequence, i.e., a sequence above the dashed line, e.g., the sequence from about amino acid 58 to 80, from about 204 to 212, and from about 266 to 282 of SEQ ID NO:77; all or part of a hydrophilic sequence, i.e., a sequence below the dashed line, e.g., the sequence of from about amino acid 89 to 96, from about 218 to 232, and from about 245 to 253 of SEQ ID NO:77; a sequence which includes a Cys, or a glycosylation site.

FIGS. 40A-40B depict a BLAST alignment of the scramblase domain (about amino acids 103 to 285 of SEQ ID NO:77) of human 33489 with a consensus amino acid sequence derived from ProDom family PD006852 (ProDomain Release 2000.1). The BLAST algorithm identifies two local alignments between the consensus amino acid sequence and human 33489. The lower sequence of the first alignment (FIG. 40A) is a consensus amino acid sequence (SEQ ID NO:83), while the upper amino acid sequence corresponds to about amino acids 149 to 285 of SEQ ID NO:77. The lower sequence of the second alignment (FIG. 40B) is a consensus amino acid sequence (SEQ ID NO:84), while the upper amino acid sequence corresponds to about amino acids 103 to 200 of SEQ ID NO:77.

FIG. 41 depicts a hydropathy plot of human 57779. Relative hydrophobic residues are shown above the dashed horizontal line, and relative hydrophilic residues are below the dashed horizontal line. Numbers corresponding to positions in the amino acid sequence of human 57779 are indicated. Above the hydropathy plot, in black bars, the location of the six cadherin domain repeats present in human 57779 are depicted. Polypeptides of the invention include fragments which include: all or part of a hydrophobic sequence, i.e., a sequence above the dashed line, e.g., the sequence from about amino acid 4 to 16, from about 676 to 701, and from about 832 to 852 of SEQ ID NO:80; all or part of a hydrophilic sequence, i.e., a sequence below the dashed line, e.g., the sequence of from about amino acid 408 to 420, from about 434 to 455, and from about 719 to 740 of SEQ ID NO:80; a sequence which includes a Cys, or a glycosylation site.

FIGS. 42A-42F depict alignments of the cadherin domains of human 57779 with a consensus amino acid sequence derived from a hidden Markov model (HMM) from PFAM. In each case, the upper sequence is the consensus amino acid sequence (SEQ ID NO:85) from the PFAM database entry PF00028 entitled “cadherin,” while the lower amino acid sequence corresponds to amino acids of SEQ ID NO:80, i.e., amino acids 25 to 120 (FIG. 42A), 134 to 229 (FIG. 42B), 243 to 337 (FIG. 42C), 354 to 444 (FIG. 42D), 458 to 554 (FIG. 42E), and 573 to 663 (FIG. 42F) of SEQ ID NO:80.

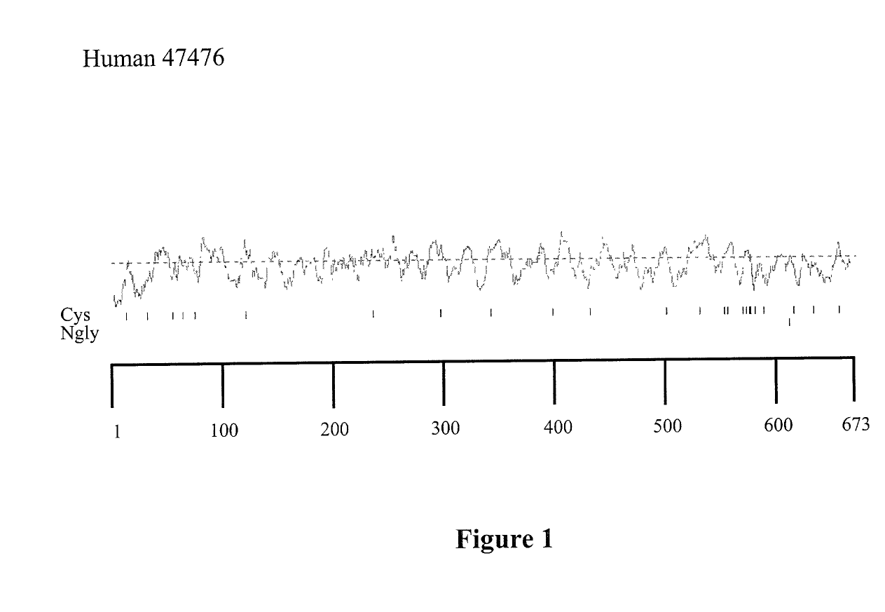

### DETAILED DESCRIPTION OF 47476, 67210, 49875, 46842, 33201, 83378, 84233, 64708, 85041, AND 84234

Human 47476

The human 47476 sequence (see SEQ ID NO:1, as recited in Example 1), which is about 3134 nucleotides long, including untranslated regions, contains a predicted methionine-initiated coding sequence of about 2022 nucleotides, including the termination codon. The coding sequence encodes a 673 amino acid protein (see SEQ ID NO:2, as recited in Example 1).

Human 47476 contains the following regions or other structural features:

a ras guanine nucleotide dissociation stimulator domain (PFAM Accession Number PF00617) located at about amino acid residues 195 to 381 of SEQ ID NO:2;

a guanine nucleotide dissociation stimulator domain N-terminal motif (SMART Accession Number SM0229) located at about amino acid residues 55 to 172 of SEQ ID NO:2;

an EF-hand calcium-binding domain (PFAM Accession Number PF00036) located at about amino acid residues 470 to 498 of SEQ ID NO:2;

a phorbol ester/diacylglycerol binding domain (C1 domain) (PFAM Accession Number PF00130) located at about amino acid residues 541 to 590 of SEQ ID NO:2;

one predicted N-glycosylation site (PS00001) located at about amino acid residues 622 to 625 of SEQ ID NO:2;

four predicted cAMP/cGMP-dependent protein kinase phosphorylation sites (PS00004) located at about amino acid rsidues 3 to 6, 7 to 10, 191 to 194 and 549 to 552 of SEQ ID NO:2;

eight predicted Protein Kinase C phosphorylation sites (PS00005) located at about amino acid residues 6 to 8, 15 to 17, 35 to 37, 233 to 235, 316 to 318, 455 to 457, 547 to 549 and 668 to 670 of SEQ ID NO:2;

fourteen predicted Casein Kinase II phosphorylation sites (PS00006) located at about amino acid residues 10 to 13, 35 to 38, 57 to 60, 210 to 213, 221 to 224, 360 to 363, 400 to 403, 418 to 421, 487 to 490, 517 to 520, 542 to 545, 552 to 555, 642 to 645 and 669 to 672 of SEQ ID NO:2;

four predicted N-myristylation sites (PS00008) located at about amino acids 232 to 237, 245 to 250, 296 to 301 and 618 to 623 of SEQ ID NO:2; and

one predicted Amidation site (PS00009) located at about amino acids 187 to 190 of SEQ ID NO:2.

A plasmid containing the nucleotide sequence encoding human 47476 (clone “Fbh47476FL”) was deposited with American Type Culture Collection (ATCC), 10801 University Boulevard, Manassas, Va. 20110-2209, on ______ and assigned Accession Number ______. This deposit will be maintained under the terms of the Budapest Treaty on the International Recognition of the Deposit of Microorganisms for the Purposes of Patent Procedure. This deposit was made merely as a convenience for those of skill in the art and is not an admission that a deposit is required under 35 U.S.C. §112.

Human 67210

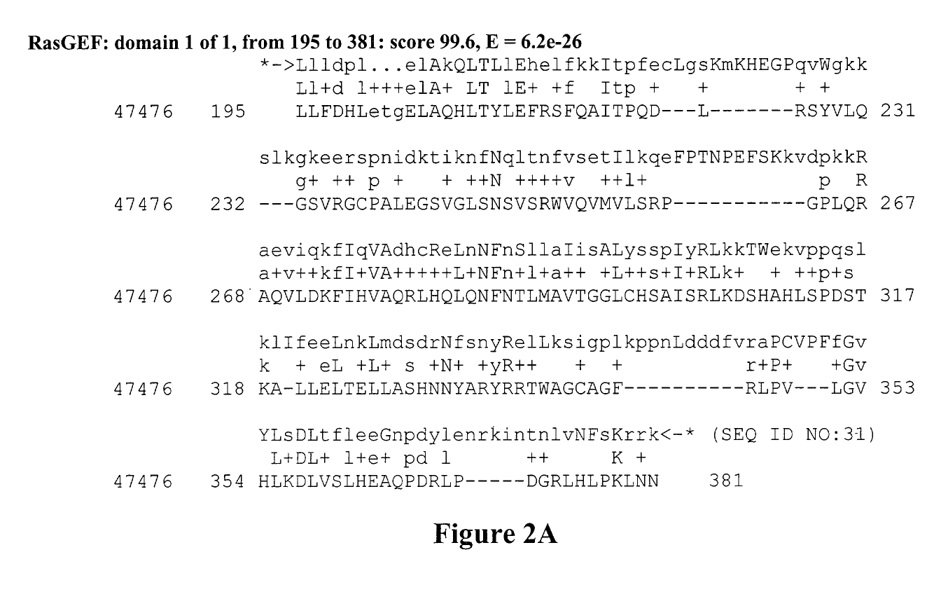

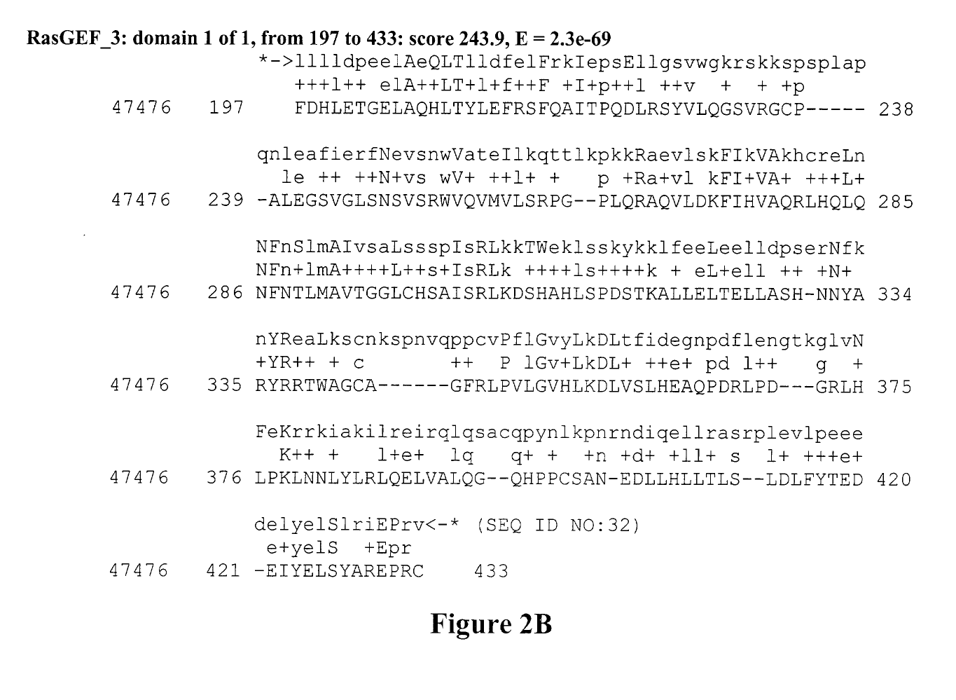

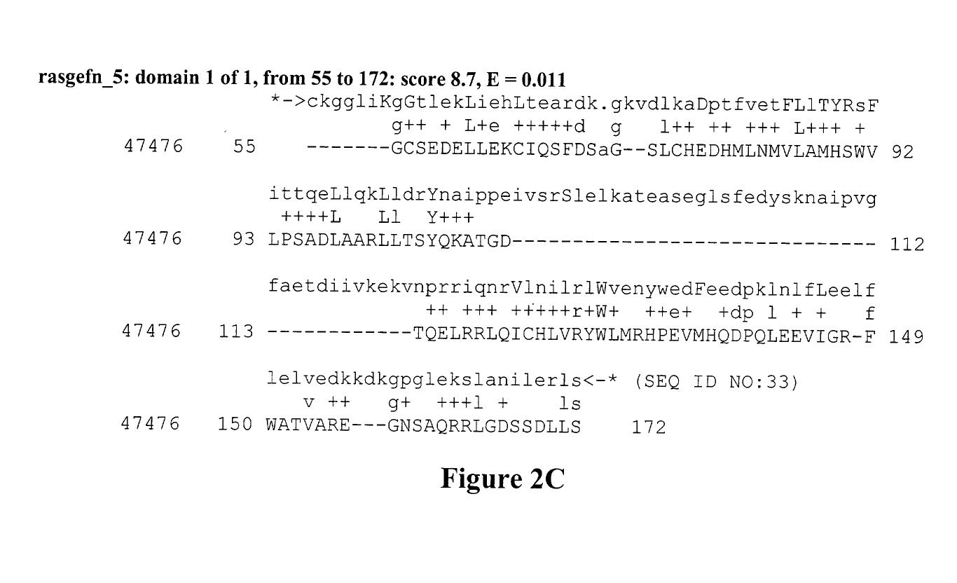

The human 67210 sequence (see SEQ ID NO:4, as recited in Example 1), which is approximately 1778 nucleotides long including untranslated regions, contains a predicted methionine-initiated coding sequence of about 1050 nucleotides, including the termination codon. The coding sequence encodes a 349 amino acid protein (see SEQ ID NO:5, as recited in Example 1). The human 67210 protein of SEQ ID NO:5 and FIG. 2 includes an amino-terminal hydrophobic amino acid sequence, consistent with a signal sequence, of about 29 amino acids (from amino acid 1 to about amino acid 29 of SEQ ID NO:5), which upon cleavage results in the production of a mature protein form.

Human 67210 contains the following regions or other structural features:

a glycosyl transferase domain (PFAM Accession Number PF01501) located at about amino acid residues 63 to 340 of SEQ ID NO:5;

a signal peptide located at about amino acids 1-29, which when cleaved gives a predicted mature protein of 319 amino acids, from about amino acid 30 to amino acid 349 of SEQ ID NO:5;

one dileucine motif located at about amino acids 3 to 4 of SEQ ID NO:5;

one predicted N-glycosylation site (PS00001) located at about amino acids 234 to 237 of SEQ ID NO:5;

one Protein Kinase C phosphorylation site (PS00005) located at about amino acids 126 to 128 of SEQ ID NO:5;

four Casein Kinase II phosphorylation sites (PS00006) located at about amino 43 to 46, 74 to 77, 127 to 130, and 189 to 192 of SEQ ID NO:5;

one tyrosine kinase phosphorylation site (PS00007) located at about amino acid 253 to 260 of SEQ ID NO:5; and

six N-myristoylation sites (PS00008) located at about amino acid 63 to 68, 86 to 91, 198 to 203, 218 to 223, 229 to 234, and 265 to 270 of SEQ ID NO:5.

A plasmid containing the nucleotide sequence encoding human 67210 (clone “Fbh67210FL”) was deposited with American Type Culture Collection (ATCC), 10801 University Boulevard, Manassas, Va. 20110-2209, on ______ and assigned Accession Number ______. This deposit will be maintained under the terms of the Budapest Treaty on the International Recognition of the Deposit of Microorganisms for the Purposes of Patent Procedure. This deposit was made merely as a convenience for those of skill in the art and is not an admission that a deposit is required under 35 U.S.C. §112.

Human 49875

The human 49875 sequence (see SEQ ID NO:7, as recited in Example 1), which is approximately 2704 nucleotides long, including untranslated regions, contains a predicted methionine-initiated coding sequence of about 1803 nucleotides, including the termination codon. The coding sequence encodes a 600 amino acid protein (see SEQ ID NO:8, as recited in Example 1).

Human 49875 contains the following regions or other structural features:

a DEAD-type helicase domain (PFAM Accession Number PF00270) located at about amino acid residues 22 to 245 of SEQ ID NO:8;

a DEAD-box subfamily ATP-dependent helicase signature motif (PS00039) located at about amino acid residues 169 to 177 of SEQ ID NO:8;

a conserved helicase C-terminal domain (PFAM Accession Number PF00271) located at about amino acid residues 281 to 363 of SEQ ID NO:8;

two N-glycosylation sites (PS00001) located at about amino acid residues 348 to 351, and 556 to 559 of SEQ ID NO:8;

five Protein Kinase C phosphorylation sites (PS00005) located at about amino acid residues 57 to 59, 224 to 226, 359 to 361, 581 to 583, and 585 to 587 of SEQ ID NO:8;

seven Casein Kinase II phosphorylation sites (PS00006) located at about amino acid residues 187 to 190, 224 to 227, 240 to 243, 507 to 510, 544 to 547, 570 to 573, and 594 to 597 of SEQ ID NO:8;

seven N-myristoylation sites (PS00008) located at about amino acid residues 7 to 12, 56 to 61, 155 to 160, 199 to 204, 229 to 234, 320 to 325, and 577 to 582 of SEQ ID NO:8;

two amidation sites (PS00009) located at about amino acid residues 510 to 513, and 581 to 584 of SEQ ID NO:8; and

one ATP/GTP-binding site motif A (P-loop) (PS00017) located at about amino acid residues 53 to 60 of SEQ ID NO:8.

A plasmid containing the nucleotide sequence encoding human 49875 (clone “Fbh49875FL”) was deposited with American Type Culture Collection (ATCC), 10801 University Boulevard, Manassas, Va. 20110-2209, on ______ and assigned Accession Number ______. This deposit will be maintained under the terms of the Budapest Treaty on the International Recognition of the Deposit of Microorganisms for the Purposes of Patent Procedure. This deposit was made merely as a convenience for those of skill in the art and is not an admission that a deposit is required under 35 U.S.C. §112.

Human 46842

The human 46842 sequence (see SEQ ID NO:10, as recited in Example 1), which is approximately 2737 nucleotides long including untranslated regions, contains a predicted methionine-initiated coding sequence of about 2505 nucleotides, including the termination codon. The coding sequence encodes a 834 amino acid protein (see SEQ ID NO:11, as recited in Example 1).

Human 46842 contains the following regions or other structural features:

a PH domain (PFAM Accession Number PF00169) located at about amino acid residues 269 to 363 SEQ ID NO:1;

an ArfGAP domain (PFAM Accession Number PF01412) located at about amino acid residues 403 to 525 of SEQ ID NO:11;

two ankyrin repeat domains (PFAM Accession Number PF00023) located at about amino acid residues 702 to 734 and 735 to 767 of SEQ ID NO:11;

an ArfGAP zinc ion coordinating motif located at about amino acid residues 421 to 440 of SEQ ID NO:11;

twelve Protein Kinase C phosphorylation sites (PS00005) located at about amino acid residues 12 to 14, 53 to 55, 102 to 104, 121 to 123, 160 to 162, 319 to 321, 347 to 349, 375 to 377, 492 to 494, 499 to 501, 544 to 546, and 549 to 551 of SEQ ID NO:11;

fourteen Casein Kinase II phosphorylation sites (PS00006) located at about amino acid residues 18 to 21, 84 to 87, 229 to 232, 253 to 256, 257 to 260, 387 to 390, 391 to 394, 500 to 503, 592 to 595, 629 to 632, 633 to 636, 645 to 648, 653 to 656, and 813 to 816 of SEQ ID NO:11;

three cAMP/cGMP-dependent protein kinase phosphorylation sites (PS00004) located at about amino acid residues 277 to 280, 493 to 496, and 577 to 580 of SEQ ID NO:11;

one tyrosine kinase phosphorylation site (PS00007) located at about amino acid residues 367 to 374 of SEQ ID NO:11;

one glycosaminoglycan attachment site (PS00002) located at about amino acid residues 610 to 613 of SEQ ID NO:11; and

ten N-myristylation sites (PS00008) located at about amino acids 42 to 47, 414 to 419, 434 to 439, 443 to 448, 449 to 454, 597 to 602, 611 to 616, 691 to 696, 726 to 731, and 808 to 813 of SEQ ID NO:11.

A plasmid containing the nucleotide sequence encoding human 46842 was deposited with American Type Culture Collection (ATCC), 10801 University Boulevard, Manassas, Va. 20110-2209, on ______ and assigned Accession Number ______. This deposit will be maintained under the terms of the Budapest Treaty on the International Recognition of the Deposit of Microorganisms for the Purposes of Patent Procedure. This deposit was made merely as a convenience for those of skill in the art and is not an admission that a deposit is required under 35 U.S.C. §112.

Human 33201

The human 33201 sequence (see SEQ ID NO:13, as recited in Example 1), which is approximately 1718 nucleotides long including untranslated regions, contains a predicted methionine-initiated coding sequence of about 1056 nucleotides, including the termination codon. The coding sequence encodes a 351 amino acid protein (see SEQ ID NO:14, as recited in Example 1).

Human 33201 contains the following regions or other structural features:

A dehydrogenase/reductase domain (PFAM Accession number PF00107) located at about amino acid residues 22 to 345 of SEQ ID NO:14;

three conserved glycine residues located at about amino acid residues 76, 93, and 224 of SEQ ID NO:14;

one Protein Kinase C phosphorylation site (PS00005) located at about amino acid residues 154 to 156 of SEQ ID NO:14;

two Casein Kinase II phosphorylation sites (PS00006) located at about amino acid residues 60 to 63 and 91 to 94 of SEQ ID NO:14;

four N-glycosylation sites (PS00001) located at about amino acids 89 to 92, 155 to 158, 235 to 238 and 281 to 284 of SEQ ID NO:14: and

eight N-myristylation sites (PS00008) located at about amino acids 16-21, 76-81, 153-158, 162-167, 168-173, 185-190, 233-238, and 335-340 of SEQ ID NO:14.

A plasmid containing the nucleotide sequence encoding human 33201 (clone “Fbh67210FL”) was deposited with American Type Culture Collection (ATCC), 10801 University Boulevard, Manassas, Va. 20110-2209, on ______ and assigned Accession Number ______. This deposit will be maintained under the terms of the Budapest Treaty on the International Recognition of the Deposit of Microorganisms for the Purposes of Patent Procedure. This deposit was made merely as a convenience for those of skill in the art and is not an admission that a deposit is required under 35 U.S.C. §112.

Human 83378

The human 83378 sequence (see SEQ ID NO:16, as recited in Example 1), which is approximately 1827 nucleotides long including untranslated regions, contains a predicted methionine-initiated coding sequence of about 1458 nucleotides, including the termination codon. The coding sequence encodes a 485 amino acid protein (see SEQ ID NO:17, as recited in Example 1).

Human 83378 contains the following regions or other structural features:

a first and a second cation efflux domain (PFAM Accession Number PF01545) located at about amino acid residues 11 to 133 and 231 to 389 of SEQ ID NO:17;

six transmembrane domains, located at about 11 to 31, 44 to 61, 79 to 98, 115 to 134, 241 to 265, and 283 to 299 of SEQ ID NO:17;

four cytoplasmic domains located at about amino acids 1 to 10 (amino terminus), 62 to 78, 135 to 240, and 300 to 485 (carboxy terminus) of SEQ ID NO:17;

three non-cytoplasmic loops located at about amino acids 32 to 43, 99 to 114, and 266 to 282 of SEQ ID NO:17;

three N-glycosylation sites (PS00001) located at about amino acid residues 377-380, 471-474, and 481-484 of SEQ ID NO:17;

one cAMP/cGMP-dependent protein kinase phosphorylation site (PS00004) located at about amino acid residues 62-65 of SEQ ID NO:17;

seven Protein Kinase C phosphorylation sites (PS00005) located at about amino acid residues 5-7,8-10, 149-151, 192-194, 342-344, 363-365, and 446-448 of SEQ ID NO:17;

five Casein Kinase II phosphorylation sites (PS00006) located at about amino acid residues 196-199, 219-222, 331-334, 446-449, and 473-476 of SEQ ID NO:17;

two tyrosine kinase phosphorylation sites (PS00007) located at about amino acid resdues 352-359 and 472-479 of SEQ ID NO:17; and

eight N-myristylation sites (PS00008) located at about amino acid residues 32-37, 54-59, 81-86, 123-128, 161-166, 168-173, 186-191, and 467-472 of SEQ ID NO:17.

A plasmid containing the nucleotide sequence encoding human 83378 (clone “Fbh83378FL”) was deposited with American Type Culture Collection (ATCC), 10801 University Boulevard, Manassas, Va. 20110-2209, on ______ and assigned Accession Number ______. This deposit will be maintained under the terms of the Budapest Treaty on the International Recognition of the Deposit of Microorganisms for the Purposes of Patent Procedure. This deposit was made merely as a convenience for those of skill in the art and is not an admission that a deposit is required under 35 U.S.C. §112.

Human 84233

The human 84233 sequence (see SEQ ID NO:19, as recited in Example 1), which is approximately 2165 nucleotides long including untranslated regions, contains a predicted methionine-initiated coding sequence of about 963 nucleotides, including the termination codon. The coding sequence encodes a 320 amino acid protein (see SEQ ID NO:20, as recited in Example 1).

Human 84233 contains the following regions or other structural features:

a cation efflux domain (PFAM Accession Number PF01545) located at about amino acid residues 25 to 310 of SEQ ID NO:20;

six transmembrane domains located at about amino acid residues 25 to 49, 58 to 74, 92 to 113, 128 to 147, 167 to 191, and 201 to 218 of SEQ ID NO:20;

four cytoplasmic domains located at about amino acid residues 1 to 24 (amino terminus), 75 to 91, 148 to 166, and 219 to 320 (carboxy terminus) of SEQ ID NO:20;

three non-cytoplasmic loops located at about amino acid residues 50 to 57, 114 to 127, and 192 to 200 of SEQ ID NO:20;

two N-glycosylation sites (PS00001) located at about amino acid residues 162-165 and 234-237 of SEQ ID NO:20;

one cAMP/cGMP-dependent protein kinase phosphorylation site (PS00004) located at about amino acid residues 81-84 of SEQ ID NO:20;

four Protein Kinase C phosphorylation sites (PS00005) located at about amino acid residues 11-13, 75-77, 80-82, and 164-166 of SEQ ID NO:20;

one Casein Kinase II phosphorylation site (PS00006) located at about amino acid residues 304-307 of SEQ ID NO:20;

one tyrosine kinase phosphorylation site (PS00007) located at about amino acid residues 13-20 of SEQ ID NO:20; and

four N-myristylation sites (PS00008) located at about amino acid residues 7-12, 42-47, 94-99, and 228-233 of SEQ ID NO:20.

A plasmid containing the nucleotide sequence encoding human 84233 (clone “Fbh84233FL”) was deposited with American Type Culture Collection (ATCC), 10801 University Boulevard, Manassas, Va. 20110-2209, on ______ and assigned Accession Number ______. This deposit will be maintained under the terms of the Budapest Treaty on the International Recognition of the Deposit of Microorganisms for the Purposes of Patent Procedure. This deposit was made merely as a convenience for those of skill in the art and is not an admission that a deposit is required under 35 U.S.C. §112.

Human 64708

The human 64708 sequence (see SEQ ID NO:22, as recited in Example 1), which is approximately 2130 nucleotides long including untranslated regions, contains a predicted methionine-initiated coding sequence of about 1386 nucleotides, including the termination codon. The coding sequence encodes a 461 amino acid protein (see SEQ ID NO:23, as recited in Example 1).

Human 64708 contains the following regions or other structural features: a first and a second cation efflux domain (PFAM Accession Number PF01545) located at about amino acid residues 55 to 153 and 227 to 320 of SEQ ID NO:23;

six transmembrane domains located at about amino acid residues 34 to 51, 58 to 82, 101 to 119, 137 to 155, 202 to 219, and 232 to 249 of SEQ ID NO:23;

four cytoplasmic domains located at about amino acid residues 1 to 33 (amino terminus), 83 to 100, 156 to 201, and 250 to 461 (carboxy terminus) of SEQ ID NO:23;

three non-cytoplasmic loops located at about amino acid residues 52 to 57, 120 to 136, and 220 to 231 of SEQ ID NO:23;

one N-glycosylation site (PS00001) located at about amino acid residues 352-355 of SEQ ID NO:23;

one cAMP/cGMP-dependent protein kinase phosphorylation site (PS00004) located at about amino acid residues 86-89 of SEQ ID NO:23;

six Protein Kinase C phosphorylation sites (PS00005) located at about amino acid residues 31-33, 84-86, 134-136, 154-156, 250-252, and 317-319 of SEQ ID NO:23;

two Casein Kinase II phosphorylation sites (PS00006) located at about amino acid residues 67-70 and 274-277 of SEQ ID NO:23;

six N-myristylation sites (PS00008) located at about amino acid residues 140-145, 185-190, 293-298, 412-417, 432-437, and 438-443 of SEQ ID NO:23; and one aminoacyl-transfer RNA synthetases class II site (PS00339) located at about amino acid residues 93-102 of SEQ ID NO:23.

A plasmid containing the nucleotide sequence encoding human 64708 (clone “Fbh64708FL”) was deposited with American Type Culture Collection (ATCC), 10801 University Boulevard, Manassas, Va. 20110-2209, on ______ and assigned Accession Number ______. This deposit will be maintained under the terms of the Budapest Treaty on the International Recognition of the Deposit of Microorganisms for the Purposes of Patent Procedure. This deposit was made merely as a convenience for those of skill in the art and is not an admission that a deposit is required under 35 U.S.C. §112.

Human 85041

The human 85041 sequence (see SEQ ID NO:25, as recited in Example 1), which is approximately 3304 nucleotides long including untranslated regions, contains a predicted methionine-initiated coding sequence of about 2298 nucleotides, including the termination codon. The coding sequence encodes a 765 amino acid protein (see SEQ ID NO:26, as recited in Example 1).

Human 85041 contains the following regions or other structural features: a cation efflux domain (PFAM Accession Number PF01545) located at about amino acid residues 419 to 733 of SEQ ID NO:26;

14 transmembrane domains located at about amino acid residues 59 to 77, 99 to 119, 129 to 145, 152 to 168, 190 to 214, 239 to 258, 267 to 288, 304 to 320, 343 to 362, 419 to 439, 486 to 505, 521 to 541, 592 to 613, and 618 to 641 of SEQ ID NO:26;

eight cytoplasmic domains located at about amino acid residues 1 to 58 (amino terminus), 120 to 128, 169 to 189, 259 to 266, 321 to 342, 438 to 485, 542 to 591, and 642 to 765 (carboxy terminus) of SEQ ID NO:26;

seven non-cytoplasmic loops located at about amino acid residues 78 to 98, 146 to 151, 215 to 238, 289 to 303, 363 to 418, 506 to 520, and 614 to 617 of SEQ ID NO:26;

one N-glycosylation site (PS00001) located at about amino acid residues 721-724 of SEQ ID NO:26;

one glycosaminoglycan attachment site (PS00002) located at about amino acid residues 143-146 of SEQ ID NO:26;

one cAMP/cGMP-dependent protein kinase phosphorylation site (PS00004) located at about amino acid residues 225-228 of SEQ ID NO:26;

eight Protein Kinase C phosphorylation sites (PS00005) located at about amino acid residues 26-28, 83-85, 224-226, 293-295, 366-368, 406-408, 675-677, and 754-756 of SEQ ID NO:26;

three Casein Kinase II phosphorylation sites (PS00006) located at about amino acid residues 262-265, 430-433, and 675-678 of SEQ ID NO:26;

one tyrosine kinase phosphorylation site (PS00007) located at about amino acid residues 750-757 of SEQ ID NO:26; and

15 N-myristylation sites (PS00008) located at about amino acid residues 14-19, 46-51, 102-107, 112-117, 144-149, 317-322, 347-352, 369-374, 437-442, 462-467, 529-534, 549-554, 579-584, 605-610, and 737-742 of SEQ ID NO:26.

A plasmid containing the nucleotide sequence encoding human 85041 (clone “Fbh85041FL”) was deposited with American Type Culture Collection (ATCC), 10801 University Boulevard, Manassas, Va. 20110-2209, on ______ and assigned Accession Number ______. This deposit will be maintained under the terms of the Budapest Treaty on the International Recognition of the Deposit of Microorganisms for the Purposes of Patent Procedure. This deposit was made merely as a convenience for those of skill in the art and is not an admission that a deposit is required under 35 U.S.C. §112.

Human 84234

The human 84234 sequence (see SEQ ID NO:28, as recited in Example 1), which is approximately 2637 nucleotides long including untranslated regions, contains a predicted methionine-initiated coding sequence of about 1131 nucleotides, including the termination codon. The coding sequence encodes a 376 amino acid protein (see SEQ ID NO:29, as recited in Example 1).

Human 84234 contains the following regions or other structural features:

a cation efflux domain (PFAM Accession Number PF01545) located at about amino acid residues 38 to 349 of SEQ ID NO:29;

six transmembrane domains located at about amino acid residues 38 to 58, 71 to 87, 105 to 123, 141 to 159, 237 to 256, and 263 to 286 of SEQ ID NO:29;

four cytoplasmic domains located at about amino acid residues 1 to 37 (amino terminus), 88 to 104, 160 to 236, and 287 to 376 (carboxy terminus) of SEQ ID NO:29;

three non-cytoplasmic loops located at about amino acid residues 59 to 70, 124 to 140, and 257 to 262 of SEQ ID NO:29;

one N-glycosylation site (PS00001) located at about amino acid residues 45 to 48 of SEQ ID NO:29;

one glycosaminoglycan attachment site (PS00002) located at about amino acid residues 170-173 of SEQ ID NO:29;

five Protein Kinase C phosphorylation sites (PS00005) located at about amino acid residues 5 to 7, 31 to 33, 34 to 36, 222 to 224, and 337 to 339 of SEQ ID NO:29;

three Casein Kinase II phosphorylation sites (PS00006) located at about amino acid residues 5 to 8, 222 to 225, and 320 to 323 of SEQ ID NO:29;

one tyrosine kinase phosphorylation site (PS00007) located at about amino acid residues 91 to 98 of SEQ ID NO:29; and

seven N-myristylation sites (PS00008) located at about amino acid residues 56 to 61, 81 to 86, 126 to 131, 169 to 174, 201 to 206, 250 to 255, and 262 to 267 of SEQ ID NO:29.

A plasmid containing the nucleotide sequence encoding human 84234 (clone “Fbh84234FL”) was deposited with American Type Culture Collection (ATCC), 10801 University Boulevard, Manassas, Va. 20110-2209, on ______ and assigned Accession Number ______. This deposit will be maintained under the terms of the Budapest Treaty on the International Recognition of the Deposit of Microorganisms for the Purposes of Patent Procedure. This deposit was made merely as a convenience for those of skill in the art and is not an admission that a deposit is required under 35 U.S.C. §112.

| | | | | ATCC |
|---|---|---|---|---|
| Gene Name | cDNA | Protein | Coding Region | accession number |
| 47476 | SEQ ID NO: 1 | SEQ ID NO: 2 | SEQ ID NO: 3 | |
| 67210 | SEQ ID NO: 4 | SEQ ID NO: 5 | SEQ ID NO: 6 | |
| 49875 | SEQ ID NO: 7 | SEQ ID NO: 8 | SEQ ID NO: 9 | |
| 46842 | SEQ ID NO: 10 | SEQ ID NO: 11 | SEQ ID NO: 12 | |
| 33201 | SEQ ID NO: 13 | SEQ ID NO: 14 | SEQ ID NO: 15 | |
| 83378 | SEQ ID NO: 16 | SEQ ID NO: 17 | SEQ ID NO: 18 | |
| 84233 | SEQ ID NO: 19 | SEQ ID NO: 20 | SEQ ID NO: 21 | |
| 64708 | SEQ ID NO: 22 | SEQ ID NO: 23 | SEQ ID NO: 24 | |
| 85041 | SEQ ID NO: 25 | SEQ ID NO: 26 | SEQ ID NO: 27 | |
| 84234 | SEQ ID NO: 28 | SEQ ID NO: 29 | SEQ ID NO: 30 | |

47476 Polypeptide Characteristics

The 47476 protein contains a significant number of structural characteristics in common with members of the ras guanine nucleotide dissociation stimulator family of proteins, the EF-hand calcium-binding domain family of proteins, and the phorbol ester/diacylglycerol binding domain (C1 domain) family of proteins. The term “family” when referring to the protein and nucleic acid molecules of the invention means two or more proteins or nucleic acid molecules having a common structural domain or motif and having sufficient amino acid or nucleotide sequence homology as defined herein. Such family members can be naturally or non-naturally occurring and can be from either the same or different species. For example, a family can contain a first protein of human origin as well as other distinct proteins of human origin, or alternatively, can contain homologues of non-human origin, e.g., rat or mouse proteins. Members of a family can also have common functional characteristics.

A ras guanine nucleotide dissociation stimulator family of proteins is characterized by a common fold. Ras guanine nucleotide dissociation stimulator proteins interact with small guanine nucleotide binding proteins such as ras and/or ras family members, e.g., rac, rho, and CDC42, and via their interaction stimulate the small guanine nucleotide binding proteins to release the guanine nucleotide to which they are bound (usually GDP). After releasing the guanine numcleotide, the small guanine nucleotide binding proteins will bind to another guanine nucleotide. Cellular concentrations of GTP are about ten-fold greater than the concentrations of GDP, so after a small guanine nucleotide binding proteins releases the guanine nucleotide to which it is bound, it wll typically bind to a GTP molecule. Thus, ras guanine nucleotide dissociation stimulator proteins act to stimulate the exchange of GDP for GTP bound to small guanine nucleotide binding proteins, which results in the activation of small guanine nucleotide binding proteins. The interaction between ras and the guanine nucleotide dissociation stimulator SOS has been described in Boriack-Sjodin et al. (1998), Nature 394:337-343, the contents of which are incorporated herein by reference. A ras guanine nucleotide dissociation stimulator can include a “ras guanine nucleotide dissociation stimulator CDC25 family signature motif”, defined by the sequence: [GAP]-[CT]-V-P-[FY]-X-X-X-X-[LIVMFY]-X-[DN]-[LIVM]. A ras guanine nucleotide dissociation stimulator CDC25 family signature motif, as defined, can be involved in triggering the dissociation of a guanine nucleotide, e.g., GDP, from a ras or ras-like protein.

A 47476 polypeptide can include a “ras guanine nucleotide dissociation stimulator domain” or regions which are homologous with a “ras guanine nucleotide dissociation stimulator domain”.

As used herein, the term “ras guanine nucleotide dissociation stimulator domain” includes an amino acid sequence of about 125 to 325 amino acid residues in length having a bit score for the alignment of the sequence to a ras guanine nucleotide dissociation stimulators domain (HMM) of at least 75. Preferably, a ras guanine nucleotide dissociation stimulator domain includes at least about 170 to 300 amino acids, more preferably about 180 to 250 amino acid residues, or about 185 to 240 amino acids and has a bit score for the alignment of the sequence to the ras guanine nucleotide dissociation stimulator domain (HMM) of at least 100, 150, 200, 225, 240, or greater. The ras guanine nucleotide dissociation stimulator domain (HMM) has been assigned the SMART Accession Number SM0147. An alternative model (HMM) for the ras guanine nucleotide dissociation stimulator domain has been assigned the PFAM Accession Number PF00617. Alignments of the ras guanine nucleotide dissociation stimulator domain (amino acids 195 to 381, or 197 to 433 of SEQ ID NO:2) of human 47476 with consensus amino acid sequences derived from a hidden Markov model according to PFAM (SEQ ID NO:31) and SMART (SEQ ID NO:32) are depicted in FIGS. 2A and 2B, respectively.

In a preferred embodiment a 47476 polypeptide or protein includes a “ras guanine nucleotide dissociation stimulator domain” or a region which includes at least about 170 to 300 amino acids, more preferably about 180 to 250 amino acid residues, or about 185 to 240 amino acid residues and has at least about 50%, 60%, 70%, 80%, 90%, 95%, 98%, 99%, or more homology with a “ras guanine nucleotide dissociation stimulator domain”, e.g., the ras guanine nucleotide dissociation stimulator domain of human 47476 (e.g., residues 195 to 381 or 197 to 433 of SEQ ID NO:2).

Some ras guanine nucleotide dissociation stimulator family members include a “ras guanine nucleotide dissociation stimulator N-terminal motif”, which is a domain that is predominantly alpha helical, is located N-terminally to the ras guanine nucleotide dissociation stimulator domain, and is believed to play a structural role (i.e., non-catalytic role) within the context of the full-length protein.

A 47476 polypeptide can include a “ras guanine nucleotide dissociation stimulator N-terminal motif” or or regions which are homologous with a “ras guanine nucleotide dissociation stimulator N-terminal motif”.

As used herein, the term “ras guanine nucleotide dissociation stimulator N-terminal motif” includes an amino acid sequence of about 75 to 250 amino acid residues in length having a bit score for the alignment of the sequence to the ras guanine nucleotide dissociation stimulator N-terminal motif domain profile (HMM) of at least 5. Preferably, a ras guanine nucleotide dissociation stimulator N-terminal motif includes at least about 100 to 200 amino acids, more preferably about 110 to 150 amino acid residues, or about 115 to 125 amino acids and has a bit score for the alignment of the sequence to the ras guanine nucleotide dissociation stimulator N-terminal motif domain profile (HMM) of at least 6, 7, 8, or greater. The ras guanine nucleotide dissociation stimulator N-terminal motif domain profile (HMM) has been assigned the SMART Accession Number SM0229. An alignment of the ras guanine nucleotide dissociation stimulator N-terminal motif (amino acids 55 to 172 of SEQ ID NO:2) of human 47476 with a consensus amino acid sequence (SEQ ID NO:33) derived from a hidden Markov model according to SMART is depicted in FIGS. 2C.

In a preferred embodiment a 47476 polypeptide or protein includes a “ras guanine nucleotide dissociation stimulator N-terminal motif” or a region which includes at least about 100 to 200 amino acids, more preferably about 110 to 150 amino acid residues, or about 115 to 125 amino acid residues and has at least about 50%, 60%, 70%, 80%, 90%, 95%, 98%, 99%, or more homology with a “ras guanine nucleotide dissociation stimulator N-terminal motif”, e.g., the ras guanine nucleotide dissociation stimulator N-terminal motif of human 47476 (e.g., residues 55 to 172 of SEQ ID NO:2).

An EF-hand family of proteins is also characterized by a common fold. EF-hand domains typically consist of a twelve-residue loop flanked on both sides by twelve residue alpha-helical domains. In an EF-hand loop, a calcium ion can be coordinated in a pentagonal bipyramidal configuration. The six residues involved in the binding are in positions 1, 3, 5, 7, 9 and 12 of the loop. The invariant Glu or Asp at position 12 provides two oxygens for liganding Ca²⁺, a bidentate ligand. An EF-hand domain can include an “EF-hand calcium-binding motif”, defined by the sequence: D-X-[DNS]-{ILVFYW}-[DENSTG]-[DNQGHRK]-{GP}-[LIVMC]-[DENQSTAGC]-X-X-[DE]-[LIVMFYW]. An EF-hand calcium-binding motif, as defined, can be involved in binding a calcium ion, e.g., a calcium ion present in the cytoplasm of a cell.

A 47476 polypeptide can include an “EF-hand calcium-binding domain” or regions homologous with an “EF-hand calcium-binding domain”.

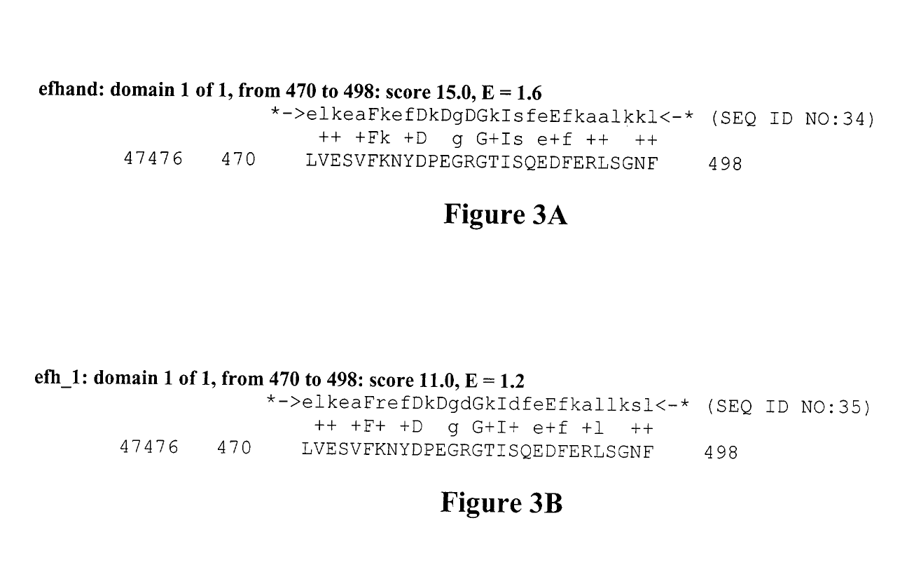

As used herein, the term “EF-hand calcium-binding domain” includes an amino acid sequence of about 20 to 60 amino acid residues in length and having a bit score for the alignment of the sequence to the EF-hand calcium-binding domain (HMM) of at least 8. Preferably, an EF-hand calcium-binding domain includes at least about 20 to 50 amino acids, more preferably about 25 to 40 amino acid residues, or about 28 to 30 amino acids and has a bit score for the alignment of the sequence to the EF-hand calcium-binding domain (HMM) of at least 10, 11, 12, 13, 14, 15, or greater. The EF-hand calcium-binding domain (HMM) has been assigned the PFAM Accession Number PF00036. An alternative model (HMM) for the ras guanine nucleotide dissociation stimulator domain has been assigned the SMART Accession Number SM0054. Alignments of the EF-hand calcium-binding domain (amino acids 470 to 498 of SEQ ID NO:2) of human 47476 with consensus amino acid sequences derived from hidden Markov models according to PFAM (SEQ ID NO:34) and SMART (SEQ ID NO:35) are depicted in FIGS. 3A and 3B, respectively.

In a preferred embodiment a 47476 polypeptide or protein includes an “EF-hand calcium-binding domain” or a region which includes at least about 20 to 50, more preferably about 25 to 40, or about 28 to 30 amino acid residues and has at least about 50%, 60%, 70%, 80%, 90%, 95%, 98%, 99%, or more homology with an “EF-hand calcium-binding domain” e.g., the EF-hand calcium-binding domain of human 47476 (e.g., residues 470 to 498 of SEQ ID NO:2).

A phorbol ester/diacylglycerol binding family of proteins is characterized by a common fold. The following sequence pattern is representative of a phorbol ester/diacylglycerol binding domain (i.e., a C1 domain): H-X-[LIVMFYW]-X(8,11)-C-X(2)-C-X(3)-[LIVMFC]-X(5,10)-C-X(2)-C-X(4)-[HD]-X(2)-C-X(5,9)-C (SEQ ID NO:60). Phorbol esters can directly stimulate Protein Kinase C, and the N-terminal region of Protein Kinase C, known as C1, has been shown to bind phorbol esters and diacylglycerols in a phospholipid and zinc-dependent fashion. The C1 region contains one or two copies of a cysteine-rich domain about 50 amino acids in length, and is essential for phorbol ester/diacylglycerol binding. The phorbol ester/diacylglycerol binding domain binds two zinc ions, the ligands of which are most likely the six cysteines and two histidines that are conserved within the phorbol ester/diacylglycerol binding domain.

A 47476 polypeptide can further include a “phorbol ester/diacylglycerol binding domain (C1 domain)” or regions homologous with a “phorbol ester/diacylglycerol binding domain (C1 domain)”.

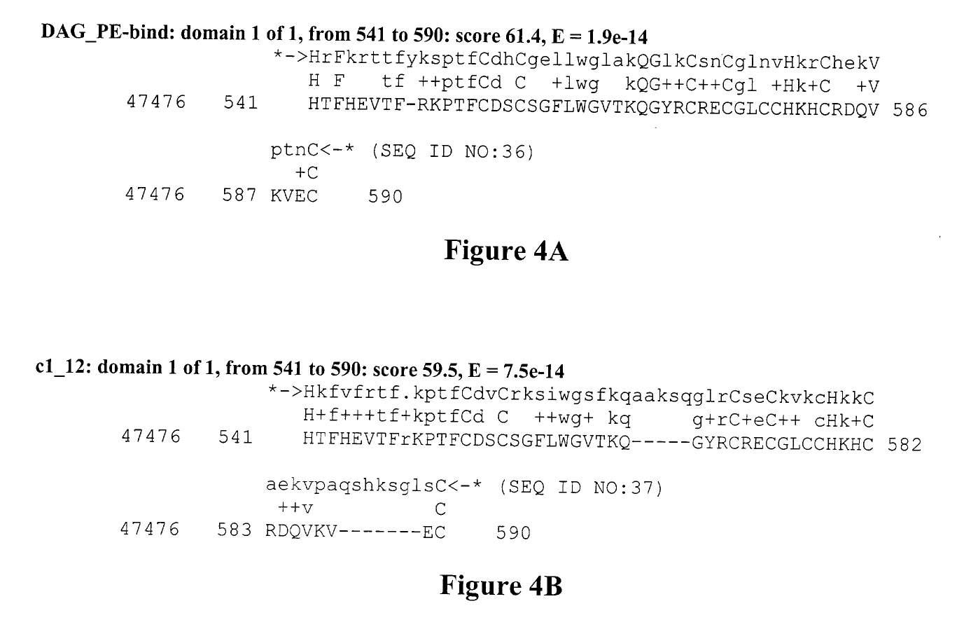

As used herein, the term “phorbol ester/diacylglycerol binding domain (C1 domain)” includes an amino acid sequence of about 30 to 100 amino acid residues in length and having a bit score for the alignment of the sequence to the phorbol ester/diacylglycerol binding domain (C1 domain) (HMM) of at least 30. Preferably, a phorbol ester/diacylglycerol binding domain (C1 domain) includes at least about 35 to 75 amino acids, more preferably about 40 to 60 amino acid residues, or about 45 to 55 amino acids and has a bit score for the alignment of the sequence to the phorbol ester/diacylglycerol binding domain (C1 domain) (HMM) of at least 40, 50, 55, 59, or greater. The phorbol ester/diacylglycerol binding domain (C1 domain) (HMM) has been assigned the PFAM Accession Number PF00130. An alternative model (HMM) for the phorbol ester/diacylglycerol binding domain has been assigned the SMART Accession Number SM0109. Alignments of the phorbol ester/diacylglycerol binding domain (C1 domain) (amino acids 541 to 590 of SEQ ID NO:2) of human 47476 with consensus amino acid sequences derived from a hidden Markov model according to PFAM (SEQ ID NO:36) and SMART (SEQ ID NO:37) are depicted in FIGS. 4A and 4B, respectively.

In a preferred embodiment a 47476 polypeptide or protein includes a “phorbol ester/diacylglycerol binding domain (C1 domain)” or a region which includes at least about 10 to 100, more preferably about 25 to 75, or about 40 to 60 amino acid residues and has at least about 50%, 60%, 70%, 80%, 90%, 95%, 98%, 99%, or more homology with a “phorbol ester/diacylglycerol binding domain (C1 domain)” e.g., the phorbol ester/diacylglycerol binding domain (C1 domain) of human 47476 (e.g., residues 541 to 590 of SEQ ID NO:2).

To identify the presence of a “Ras guanine nucleotide dissociation stimulator domain”, a “ras guanine nucleotide dissociation stimulator N-terminal motif”, an “EF-hand calcium binding domain”, or a “phorbol ester/diacylglycerol bining domain” in a 47476 protein sequence, and make the determination that a polypeptide or protein of interest has a particular profile, the amino acid sequence of the protein can be searched against the PFAM database of HMMs (e.g., the PFAM database, release 2.1) using the default parameters. For example, the hmmsf program, which is available as part of the HMMER package of search programs, is a family specific default program for MILPAT0063 and a score of 15 is the default threshold score for determining a hit. Alternatively, the threshold score for determining a hit can be lowered (e.g., to 8 bits). A description of the PFAM database can be found in Sonhammer et al. (1997) Proteins 28(3):405-420 and a detailed description of HMMs can be found, for example, in Gribskov et al. (1990) Meth. Enzymol. 183:146-159; Gribskov et al. (1987) Proc. Natl. Acad. Sci. USA 84:4355-4358; Krogh et al. (1994) J. Mol. Biol. 235:1501-1531; and Stultz et al. (1993) Protein Sci. 2:305-314, the contents of which are incorporated herein by reference. A search was performed against the HMM database resulting in the identification of: a “ras guanine nucleotide dissociation stimulator domain” in the amino acid sequence of human 47476 located at about residues 195 to 381 of SEQ ID NO:2 (see FIG. 2A); an “EF-hand calcium binding domain” in the amino acid sequence of human 47476 located at about residues 470 to 498 of SEQ ID NO:2 (see FIG. 3A); and a “phorbol ester/diacylglycerol binding domain” in the amino acid sequence of human 47476 located at about residues 541 to 590 of SEQ ID NO:2 (see FIG. 4A).

Alternatively, to identify the presence of a “ras guanine nucleotide dissociation stimulator domain”, a “ras guanine nucleotide dissociation stimulator N-terminal motif”, an “EF-hand calcium binding domain”, or a “phorbol ester/diacylglycerol binding domain” in a 47476 protein sequence, and make the determination that a polypeptide or protein of interest has a particular profile, the amino acid sequence of the protein can be searched against a SMART database (Simple Modular Architecture Research Tool) of HMMs as described in Schultz et al. (1998), Proc. Natl. Acad. Sci. USA 95:5857 and Schultz et al. (200) Nucl. Acids Res 28:231, the contents of which are incorporated herein by reference. The database contains domains identified by profiling with the hidden Markov models of the HMMer2 search program (R. Durbin et al. (1998) Biological sequence analysis: probabilistic models of proteins and nucleic acids. Cambridge University Press). The database also is extensively annotated and monitored by experts to enhance accuracy. A search was performed against the HMM database resulting in the identification of: a “ras guanine nucleotide dissociation stimulator domain” in the amino acid sequence of human 47476 at about residues 195 to 381 of SEQ ID NO:2 (see FIG. 2B); a “ras guanine nucleotide dissociation stimulator N-terminal motif” in the amino acid sequence of human 47476 at about residues 55 to 172 of SEQ ID NO:2 (see FIG. 2C); an “EF-hand calcium binding domain” in the amino acid sequence of human 47476 at about residues 470 to 498 of SEQ ID NO:2 (see FIG. 3B); and a “phorbol ester/diacylglycerol binding domain” in the amino acid sequence of human 47476 at about residues 541 to 590 of SEQ ID NO:2 (see FIG. 4B).

A 47476 family member can include at least one ras guanine nucleotide dissociation stimulator domain, at least one ras guanine nucleotide dissociation sitmulator N-terminal motif, at least one EF-hand calcium-binding domain, and at least one phorbol ester/diacylglycerol binding domain (C1 domain). Furthermore, a 47476 family member can include: at least one predicted N-glycosylation sites (PS00001); at least one, two, three, and preferably four predicted cAMP- and cGMP-dependent protein kinase phosphorylation sites (PS00004); at least one, two, three, four, five, six, seven, and preferably eight predicted protein kinase C phosphorylation sites (PS00005); at least one, two, three, four, five, six, seven, eight, nine, ten, eleven, twelve, thirteen and preferably fourteen predicted casein kinase II phosphorylation sites (PS00006); at least one, two, three, and preferably four predicted N-myristylation sites (PS00008); at least one predicted amidation site (PS00009); and at least one predicted phorbol ester/diacylglycerol binding domain (PS00479).

As the 47476 polypeptides of the invention may modulate 47476-mediated activities, they may be useful as of for developing novel diagnostic and therapeutic agents for 47476-mediated or related disorders, as described below.

As used herein, a “47476 activity”, “biological activity of 47476” or “functional activity of 47476”, refers to an activity exerted by a 47476 protein, polypeptide or nucleic acid molecule. For example, a 47476 activity can be an activity exerted by 47476 in a physiological milieu on, e.g., a 47476-responsive cell or on a 47476 substrate, e.g., a protein substrate. A 47476 activity can be determined in vivo or in vitro. In one embodiment, a 47476 activity is a direct activity, such as an association with a 47476 target molecule. A “target molecule” or “binding partner” is a molecule with which a 47476 protein binds or interacts in nature. In an exemplary embodiment, a 47476 polypeptide or protein has the ability to interact with, and thereby activate, ras and/or a ras-like protein.

A 47476 activity can also be an indirect activity, e.g., a cellular signaling activity mediated by another protein, e.g., ras or a ras-like protein, that the 47476 protein interacts with. Based on the above-described sequence similarities, the 47476 molecules of the present invention are predicted to have similar biological activities as ras guanine nucleotide dissociation stimulator (also known as “ras guanine nucleotide exchange factor”) family members. For example, the 47476 proteins of the present invention can have one or more of the following activities: (1) the ability to stimulate the exchange of guanine nucleotides (e.g., GTP for GDP) by another protein, e.g., ras or a ras-like protein; (2) the ability to bind calcium ions; (3) the ability to bind zinc ions; (4) the ability to bind the second messenger, diacylglycerol; (5) the ability to bind analogs of diacylglycerol, such as phorbol esters; (6) the ability to activate members of the ras superfamily of proteins; (7) the ability to influence the acitivity of signaling pathways that include ras or ras-like proteins, e.g., growth factor signaling pathways or cellular adhesion signaling pathways; (8) the ability to influence cellular proliferation; (9) the ability to influence cellular differentiation; (10) the ability to influence cellular adhesion; (11) the ability to influence cell migration; or (12) has the ability to antagonize or inhibit, competitively or non-competitively, any of 1-11.

In addition, the 47476 molecules of the invention can be expected to function in tissues in which they are expressed. For example, human 47476 is expressed in blood cells (e.g., erythroid cells, megakaryocytes, neutrophils, periperal blood mononuclear cells), bone marrow mononuclear cells, spleen, and lung (see Example 2). Thus, the 47476 molecules can act as novel diagnostic targets and therapeutic agents for controlling aberrant or deficient signal transduction resulting in, e.g., hematopoeitic disorders, including, e.g., blood clotting disorders, autoimmune disorders, or disorders related to an inability to clear infections (e.g., viral viral or bacterial infections), as well as disorders related to abnormal cellular proliferation or differentiation, e.g., leukemias.

The 67210 protein contains a significant number of structural characteristics in common with members of the glycosyl transferase family. The term “family” when referring to the protein and nucleic acid molecules of the invention means two or more proteins or nucleic acid molecules having a common structural domain or motif and having sufficient amino acid or nucleotide sequence homology as defined herein. Such family members can be naturally or non-naturally occurring and can be from either the same or different species. For example, a family can contain a first protein of human origin as well as other distinct proteins of human origin, or alternatively, can contain homologues of non-human origin, e.g., rat or mouse proteins. Members of a family can also have common functional characteristics.

The glycosyl transferase family comprises a number of related enzymes that are capable of catalyzing the synthesis of glycoconjugates, including glycolipids, glycoproteins, and polysaccharides, by transferring an activated mono- or oligosaccharide residue to an existing acceptor molecule for the initiation or elongation of the carbohydrate chain. The acceptor can be a lipid, a protein, a heterocyclic compound, or another carbohydrate residue. Glycosyltransferases can be divided into numerous subfamilies based upon their specificity for sugar moieties and acceptor molecules. The glycosyltransferase domain of human 67210 bears similarity to a subfamily designated “group 8” glycosyltransferases. These enzymes comprise a subfamily whose members are involved in lipopolysaccharide biosynthesis and glycogen synthesis. Members of this family include lipopolysaccharide galactosyl-transferase, lipopolysaccharide glucosyltransferase 1, and glycogenin glucosyltransferase. Thus, this family includes enzymes critical for the proper function of many physiological systems, including carbohydrate and lipid metabolism, and cellular proliferation and differentiation.

A 67210 polypeptide can include a “glycosyltransferase domain” or regions homologous with a “glycosyltransferase domain”.

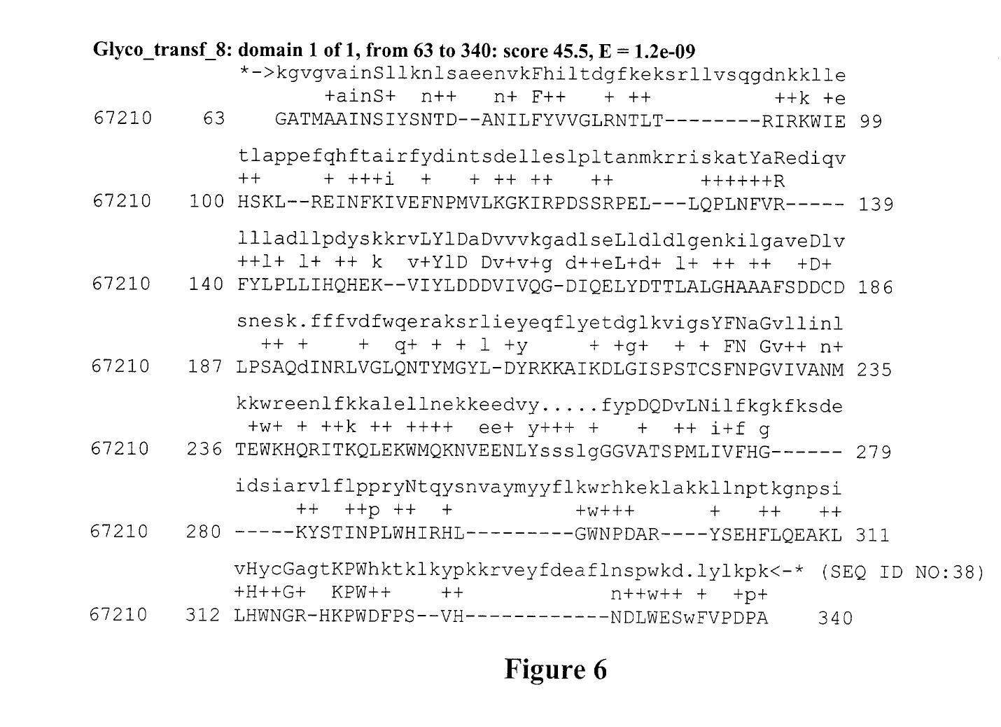

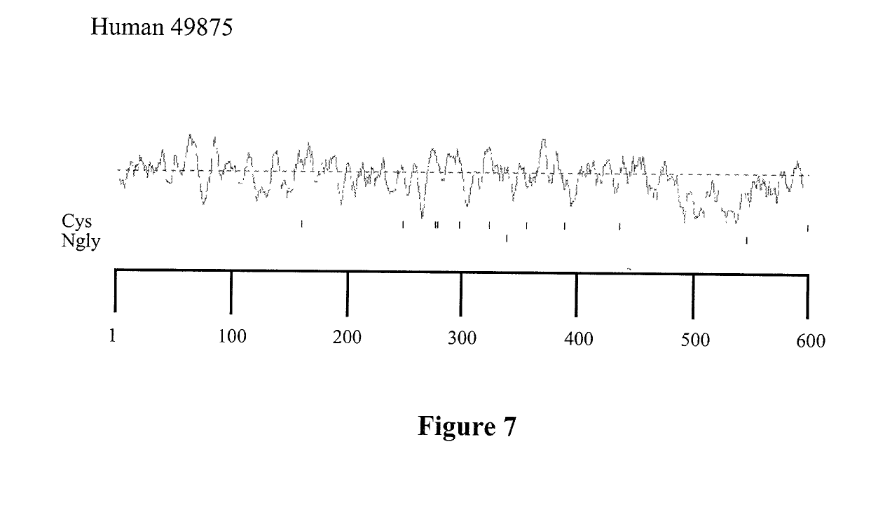

As used herein, the term “glycosyl transferase domain” includes an amino acid sequence of about 100 to 450 amino acid residues in length, having a bit score for the alignment of the sequence to the glycosyltransferase domain (HMM) of at least 25. Preferably, a glycosyl transferase domain includes about 200 to 350 amino acid residues, or more preferably about 250 to 300 amino acids and has a bit score for the alignment of the sequence to the glycosyltransferase domain (HMM) of at least 30, 35, more 40, or more. The glycosyl transferase domain (HMM) has been assigned the PFAM Accession Number PF01501. An alignment of glycosyl transferase domain (about amino acids 63 to 340 of SEQ ID NO:5) of human 67210 with a consensus amino acid sequence (SEQ ID NO:38) derived from a hidden Markov model is depicted in FIG. 6.

In preferred embodiments, a 67210 polypeptide or protein has a “glycosyl transferase domain” or a region which includes at least about 100 to 450, more preferably about 200 to 350, or 250 to 300 amino acid residues and has at least about 60%, 70%, 80%, 90%, 95%, 98%, 99%, or more homology with a “glycosyltransferase,” e.g., the glycosyltransferase domain of human 67210 (e.g., residues 63 to 340 of SEQ ID NO:5).

To identify the presence of a “glycosyltransferase” domain in a 67210 protein sequence, and make the determination that a polypeptide or protein of interest has a particular profile, the amino acid sequence of the protein can be searched against the PFAM database of HMMs (e.g., the Pfam database, release 2.1) using the default parameters. For example, the hmmsf program, which is available as part of the HMMER package of search programs, is a family specific default program for MILPAT0063 and a score of 15 is the default threshold score for determining a hit. Alternatively, the threshold score for determining a hit can be lowered (e.g., to 8 bits). A description of the PFAM database can be found in Sonhammer et al. (1997) Proteins 28(3):405-420 and a detailed description of HMMs can be found, for example, in Gribskov et al. (1990) Meth. Enzymol. 183:146-159; Gribskov et al. (1987) Proc. Natl. Acad. Sci. USA 84:4355-4358; Krogh et al. (1994) J. Mol. Biol. 235:1501-1531; and Stultz et al. (1993) Protein Sci. 2:305-314, the contents of which are incorporated herein by reference. A search was performed against the HMM database resulting in the identification of a “glycosyl transferase” domain in the amino acid sequence of human 67210 located at about residues 63 to 340 of SEQ ID NO:5 (see FIG. 6).

A 67210 peptide can further include a signal sequence. As used herein, a “signal peptide” or “signal sequence” refers to a peptide of about 20 to 60, preferably about 25 to 55, or more preferably about 29 amino acid residues in length which occurs at the N-terminus of secretory and integral membrane proteins and which contains a majority of hydrophobic amino acid residues. For example, a signal sequence contains at least about 20 to 60, preferably about 25 to 55, or more preferably about 30 amino acid residues, and has at least about 40-70%, preferably about 50-65%, and more preferably about 55-60% hydrophobic amino acid residues (e.g., alanine, valine, leucine, isoleucine, phenylalanine, tyrosine, tryptophan, or proline). Such a “signal sequence”, also referred to in the art as a “signal peptide”, serves to direct a protein containing such a sequence to a lipid bilayer. For example, in one embodiment, a 67210 protein contains a signal sequence located at about amino acid residues 1 to 29 of SEQ ID NO:5. The “signal sequence” is cleaved during processing of the mature protein, and the mature 67210 protein corresponds to about amino acid residues 30 to 349 of SEQ ID NO:5.

A 67210 family member can include at least one glycosyl transferase domain and at least one signal peptide. A 67210 family member can further include: at least one dileucine motif; at least one predicted N-glycosylation site (PS00001); at least one predicted Protein Kinase C phosphorylation site (PS00005); at least one, two, three, preferably four predicted Casein Kinase II phosphorylation sites (PS00006); at least one predicted tyrosine kinase phosphorylation site (PS00007); and at least one, two, three, four, five, preferably six predicted N-myristoylation sites (PS00008).

As the 67210 polypeptides of the invention may modulate 67210-mediated activities, they may be useful as of for developing novel diagnostic and therapeutic agents for 67210-mediated or related disorders, as described below.

As used herein, a “67210 activity”, “biological activity of 67210” or “functional activity of 67210”, refers to an activity exerted by a 67210 protein, polypeptide or nucleic acid molecule. For example, a 67210 activity can be an activity exerted by 67210 in a physiological milieu on, e.g., a 67210-responsive cell or on a 67210 substrate, e.g., a protein substrate. A 67210 activity can be determined in vivo or in vitro. In one embodiment, a 67210 activity is a direct activity, such as an association with a 67210 target molecule. A “target molecule” or “binding partner” is a molecule with which a 67210 protein binds or interacts in nature. In an exemplary embodiment, 67210 is an enzyme that modifies (e.g., adds a sugar residue to) a lipid, protein, or carbohydrate substrate.

A 67210 activity can also be an indirect activity, e.g., a cellular signaling activity mediated by interaction of the 67210 protein with a 67210 receptor. The features of the 67210 molecules of the present invention can provide similar biological activities as glycosyltransferase family members. For example, the 67210 proteins of the present invention can have one or more of the following activities: (1) catalyzes the transfer of an activated sugar residue to an acceptor molecule; (2) catalyzes the processing, folding, and secretion of glycoproteins; (3) catalyzes carbohydrate metabolism, e.g., lipopolysaccharide biosynthesis and glycogen synthesis; (4) catalyzes lipid metabolism; (5) modulates cell-cell interactions, e.g., between endothelial cells and blood cells; (6) modulates cell-matrix adhesive interactions; (7) modulates signal transduction, e.g., growth factor signaling; (8) modulates cell proliferation and/or differentiation; (9) modulates tumor cell growth, invasion and/or metastasis; (10) regulates myelin formation; (11) regulates viral and microbial adhesion; (12) modulates oligodendrocyte development; (13) controls sperm-egg binding; (14) regulates evasion of immune detection; (15) modulates xenograft rejection; or (16) has the ability to antagonize or inhibit, competitively or non-competitively, any of 1-15.

In addition, the 67210 molecules of the invention can be expected to function in tissues in which they are expressed. For example, human 67210 is expressed in cardiovascular tissues (e.g., arteries and smooth muscle cells), neural tissues (e.g., the brain cortex), and breast and ovary tissues (see Example 2). Thus, the 67210 molecules can act as novel diagnostic targets and therapeutic agents for controlling disorders of metabolic imbalance (e.g., disorders of lipopolysaccharide biosynthesis or glycogen synthesis), immunological disorders (e.g., autoimmune disorders or disorders associated with an inability to clear an infection, e.g., a viral or bacterial infection), cardiovascular disorders, nueurological disorders, or cellular proliferation and/or differentiation disorders, e.g., cancer.

49875

The 49875 protein contains a significant number of structural characteristics in common with members of the DEAD type helicase family. The term “family” when referring to the protein and nucleic acid molecules of the invention means two or more proteins or nucleic acid molecules having a common structural domain or motif and having sufficient amino acid or nucleotide sequence homology as defined herein. Such family members can be naturally or non-naturally occurring and can be from either the same or different species. For example, a family can contain a first protein of human origin as well as other distinct proteins of human origin, or alternatively, can contain homologues of non-human origin, e.g., rat or mouse proteins. Members of a family can also have common functional characteristics.

A DEAD type helicase family of proteins is characterized by a common DEAD-box subfamily ATP-dependent helicase signature motif. The DEAD-type helicase family comprises a number of related enzymes that share high structural homology and a common catalytic mechanism whereby the enzyme converts the energy from ATP hydrolysis into the mechanical energy required for unwinding of nucleic acid duplexes. For example, DEAD-type helicases catalyze the unwinding of ribonucleic acids during RNA splicing. Thus, this family includes enzymes critical for the proper function of many physiological systems, including replication, transcription, and cellular proliferation and differentiation.

A 49875 polypeptide can include a “DEAD type helicase domain” or regions homologous with a “DEAD type helicase domain”.

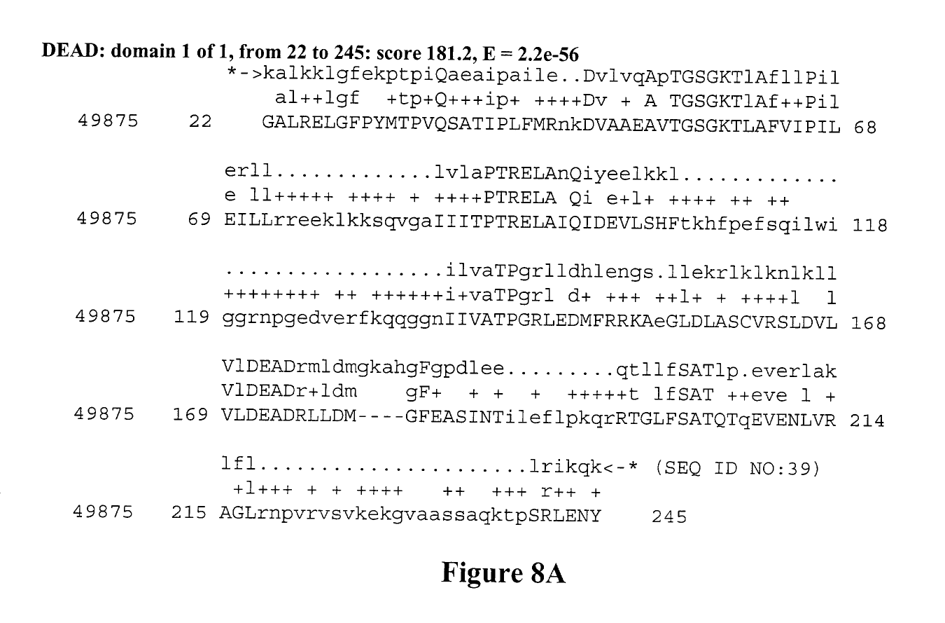

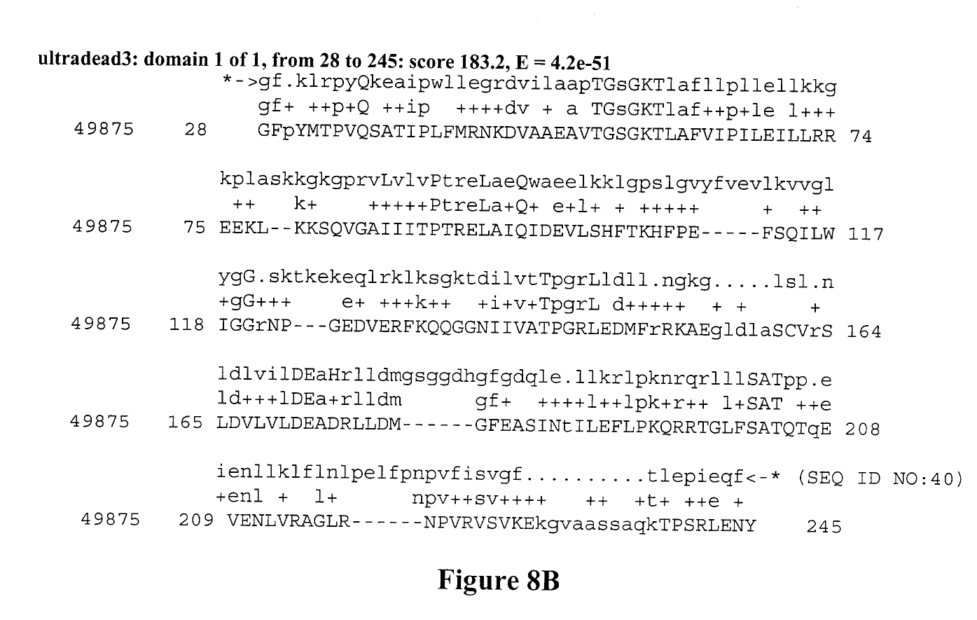

As used herein, the term “DEAD-type helicase domain” is an amino acid sequence of at least 100 amino acid residues in length and having a bit score for the alignment of the sequence to the DEAD type helicase domain (HMM) of at least 100. Preferably, a DEAD type helicase domain includes an amino acid sequence of about 100 to 350 amino acid residues in length, more preferably about 200 to 250 amino acid residues, or about 215 to 235 amino acids and having a bit score for the alignment of the sequence to the DEAD type helicase domain (HMM) of at least 100, preferably 150, and most preferably 180 or more. The DEAD type helicase domain (HMM) has been assigned the PFAM Accession Number PF00270 (http;//genome.wustl.edu/Pfam/.html). An alternative model (HMM) for the DEAD type helicase domain has been assigned the SMART Accession Number SM0487. Alignments of the DEAD type helicase domain (amino acids 22 to 245 or 28 to 245 of SEQ ID NO:8) of human 49875 with a consensus amino acid sequence derived from a hidden Markov model according to PFAM (SEQ ID NO:39) or according to SMART (SEQ ID NO:40) are depicted in FIGS. 8A and 8B, respectively.

In a preferred embodiment 49875 polypeptide or protein has a “DEAD-type helicase domain” or a region which includes at least about 100 to 350, more preferably about 200 to 250, or 215 to 235 amino acid residues and has at least about 60%, 70%, 80%, 90%, 95%, 98%, 99%, or more homology with a “DEAD-type helicase,” e.g., the DEAD-type helicase domain of human 49875 (e.g., residues 22 to 245 of SEQ ID NO:8).

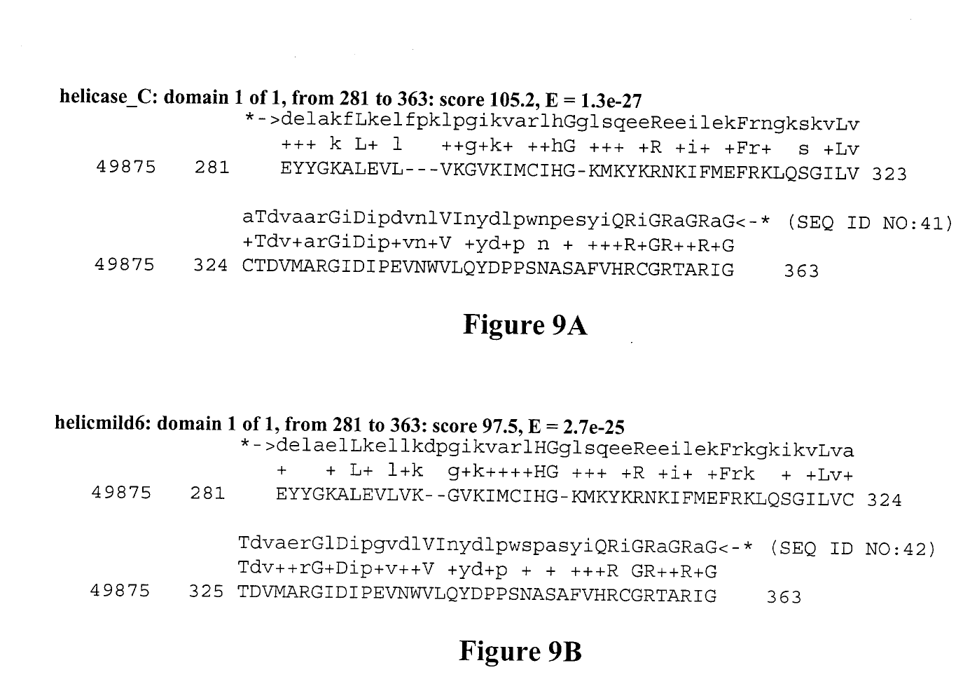

As used herein, the term “conserved helicase C-terminal domain” is an amino acid sequence of at least 30 amino acid residues in length having a bit score for the alignment of the sequence to the C-terminal helicase domain (HMM) of at least 50. A “conserved helicase C-terminal domain” preferably includes an amino acid sequence of about 20 to 140 amino acid residues in length, more preferably about 40 to 120 amino acid residues, or about 60 to 100 amino acids and having a bit score for the alignment of the sequence to the C-terminal helicase domain (HMM) of at least 50, preferably 80, and most preferably 90 or more. The conserved helicase C-terminal domain (HMM) has been assigned the PFAM Accession Number PF00271). An alternative model (HMM) for the conserved helicase C-terminal domain has been assigned the SMART Accession Number SM0490. Alignments of the conserved helicase C-terminal domain (amino acids 281 to 363 of SEQ ID NO:8) of human 49875 with consensus amino acid sequences derived from a hidden Markov model derived from PFAM (SEQ ID NO:41) and SMART (SEQ ID NO:42) are depicted in FIGS. 9A and 9B.

In a preferred embodiment 49875 polypeptide or protein has a “conserved helicase C-terminal domain” or a region which includes at least about 20 to 140, more preferably about 40 to 120, or 60 to 100 amino acid residues and has at least about 60%, 70% 80% 90% 95%, 99%, or 100% homology with a “conserved helicase C-terminal domain,” e.g., the conserved helicase C-terminal domain of human 49875 (e.g., residues 281 to 363 of SEQ ID NO:8).

To identify the presence of a “DEAD type helicase domain” or a “conserved helicase C-terminal domain” in a 49875 protein sequence, and make the determination that a polypeptide or protein of interest has a particular profile, the amino acid sequence of the protein can be searched against the PFAM database of HMMs (e.g., the PFAM database, release 2.1) using the default parameters. For example, the hmmsf program, which is available as part of the HMMER package of search programs, is a family specific default program for MILPAT0063 and a score of 15 is the default threshold score for determining a hit. Alternatively, the threshold score for determining a hit can be lowered (e.g., to 8 bits). A description of the PFAM database can be found in Sonhammer et al. (1997) Proteins 28(3):405-420 and a detailed description of HMMs can be found, for example, in Gribskov et al. (1990) Meth. Enzymol. 183:146-159; Gribskov et al. (1987) Proc. Natl. Acad. Sci. USA 84:4355-4358; Krogh et al. (1994) J. Mol. Biol. 235:1501-1531; and Stultz et al. (1993) Protein Sci. 2:305-314, the contents of which are incorporated herein by reference. A search was performed against the HMM database resulting in the identification of: a “DEAD type helicase domain” in the amino acid sequence of human 49875 at about residues 22 to 245 of SEQ ID NO:8 (see FIG. 8A); and a “conserved helicase C-terminal domain” in the amino acid sequence of human 49875 at about residues 281 to 363 of SEQ ID NO:8 (see FIG. 9A).

Alternatively, to identify the presence of a “DEAD type helicase domain” or a “conserved helicase C-terminal domain” in a 49875 protein sequence, and make the determination that a polypeptide or protein of interest has a particular profile, the amino acid sequence of the protein can be searched against a SMART database (Simple Modular Architecture Research Tool) of HMMs as described in Schultz et al. (1998), Proc. Natl. Acad. Sci. USA 95:5857 and Schultz et al. (200) Nucl. Acids Res 28:231, the contents of which are incorporated herein by reference. The database contains domains identified by profiling with the hidden Markov models of the HMMer2 search program (R. Durbin et al. (1998) Biological sequence analysis: probabilistic models of proteins and nucleic acids. Cambridge University Press). The database also is extensively annotated and monitored by experts to enhance accuracy. A search was performed against the HMM database resulting in the identification of: a “DEAD type helicase domain” in the amino acid sequence of human 49875 at about residues 28 to 245 of SEQ ID NO:8 (see FIG. 8B); and a “conserved helicase C-terminal domain” (“helicase_C”) in the amino acid sequence of human 49875 at about residues 281 to 363 of SEQ ID NO:8 (see FIG. 9B).

In some embodiments, a 49875 protein includes at least one DEAD-box subfamily ATP-dependent helicase signature motif. As used herein, a “DEAD-box subfamily ATP-dependent helicase signature motif” includes a sequence of at least six amino acid residues defined by the sequence: [LIVMF]-[LIVMF]-D-E-A-D-[RKEN]-X-[LIVMFYGSTN] (SEQ ID NO:61). A DEAD-box subfamily ATP-dependent helicase signature motif, as defined, can be involved in unwinding a nucleic acid double helix, e.g., a DNA or RNA double helix. More preferably, a DEAD-box subfamily ATP-dependent helicase signature motif includes 7, 8, and most preferably 9 amino acid residues. The DEAD-box subfamily ATP-dependent helicase signature motif have been given the PROSITE Accession Number PS00039.

In preferred embodiments, a 49875 polypeptide or protein has at least one DEAD-box subfamily ATP-dependent helicase signature motif, or a region which includes at least 6, 7, 8, or even 9 amino acid residues and has at least 70%, 80%, 90%, or 100% homology with a “DEAD-box subfamily ATP-dependent helicase signature motif”, e.g., DEAD-box subfamily ATP-dependent helicase signature motif of human 49875, e.g., about amino acid residues 169 to 177 of SEQ ID NO:8.

A 49875 family member can include at least one DEAD type helicase domain, and at least one conserved helicase C-terminal domain. Furthermore, a 49875 family member can include: at least one DEAD-box subfamily ATP-dependent helicase signature motif (PS00039); at least one, preferably two predicted N-glycosylation sites (PS00001); at least one, two, three, preferably four predicted protein kinase C phosphorylation sites (PS00005); at least one, two, three, four, five, six, preferably seven predicted casein kinase II phosphorylation sites (PS00006); at least one, two, three, four, five, six, preferably seven predicted N-myristylation sites (PS00008); at least one, preferably two predicted amidation sites (PS00009); and at least one ATP/GTP binding site (P-loop) (PS00017).

As the 49875 polypeptides of the invention may modulate 49875-mediated activities, they may be useful as of for developing novel diagnostic and therapeutic agents for 49875-mediated or related disorders, as described below.

As used herein, a “49875 activity”, “biological activity of 49875” or “functional activity of 49875”, refers to an activity exerted by a 49875 protein, polypeptide or nucleic acid molecule. For example, a 49875 activity can be an activity exerted by 49875 in a physiological milieu on, e.g., a 49875-responsive cell or on a 49875 substrate, e.g., ATP or a nucleic acid. A 49875 activity can be determined in vivo or in vitro. In one embodiment, a 49875 activity is a direct activity, such as an association with a 49875 target molecule. A “target molecule” or “binding partner” is a molecule with which a 49875 protein binds or interacts in nature, e.g., ATP or a nucleic acid. In an exemplary embodiment, 49875 is an unwinding enzyme for a nucleic acid substrate, e.g., a double stranded nucleic acid substrate, e.g., double stranded RNA.

The features of the 49875 molecules of the present invention can provide similar biological activities as DEAD type helicase family members. For example, the 49875 proteins of the present invention can have one or more of the following activities: (1) binds nucleic acid molecules, e.g., RNA; (2) binds and hydrolyzes nucleoside 5′-triphosphate (NTP), e.g., ATP or GTP; (3) catalyzes the separation of two complementary strands of a duplex nucleic acid molecules; (4) modulates replication; (5) modulates recombination; (6) modulates transcription; (7) modulates translation; (8) modulates RNA splicing; (9) modulates nucleic acid metabolism; (10) acts as a transcriptional regulator; or (11) has the ability to antagonize or inhibit, competitively or non-competitively, any of 1-10. As a result, the 49875 protein may have a critical function in one or more of the following physiological processes: (1) replication, e.g., eukaryotic or viral replication; (2) regulation of transcription; or (3) cell proliferation and differentiation.

In addition, the 49875 molecules of the invention can be expected to function in tissues in which they are expressed. For example, human 49875 is expressed in ovary and lung tissues, and the expression is upregulated in ovary and lung tumors (see Example 2). Thus, the 49875 molecules can act as novel diagnostic targets and therapeutic agents for controlling disorders of proliferation and/or differentiation, e.g., cancer, or immunological disorders.

46842

The 46842 protein contains a significant number of structural characteristics in common with members of the centaurin family. The 46842 protein has the organization of protein domains typical of centaurins, especially centaurin-γ family members. This domain structure is as follows: a PH domain, an ArfGAP domain, a first ankyrin domain, and a second ankyrin domain. The term “family” when referring to the protein and nucleic acid molecules of the invention means two or more proteins or nucleic acid molecules having a common structural domain or motif and having sufficient amino acid or nucleotide sequence homology as defined herein. Such family members can be naturally or non-naturally occurring and can be from either the same or different species. For example, a family can contain a first protein of human origin as well as other distinct proteins of human origin, or alternatively, can contain homologues of non-human origin, e.g., rat or mouse proteins. Members of a family can also have common functional characteristics.

PH domains are small domains found in a diverse class of proteins, including those involved in intracellular signaling and the cytoskeleton. The domain has been implicated in binding the βγ subunit of heterotrimeric G proteins, lipids (e.g., ascorbyl sterates, and phosphoinositols), and phosphoserine and phosphothreonine. The ligand specificity of PH domains can vary from domain to domain. The structures of multiple PH domains have been determined (for a review, see Riddihough (1994) Nat. Struct. Biol. 1:755-757). From analysis of such structures, it is evident that the PH domains represent a conserved fold consisting of two perpendicular β-sheets followed by an amphipathic α-helix despite the lack of absolutely conserved residues. However, the loop regions differ greatly in length and composition.

A 46842 polypeptide can include a “PH domain” or regions homologous with a “PH domain”.

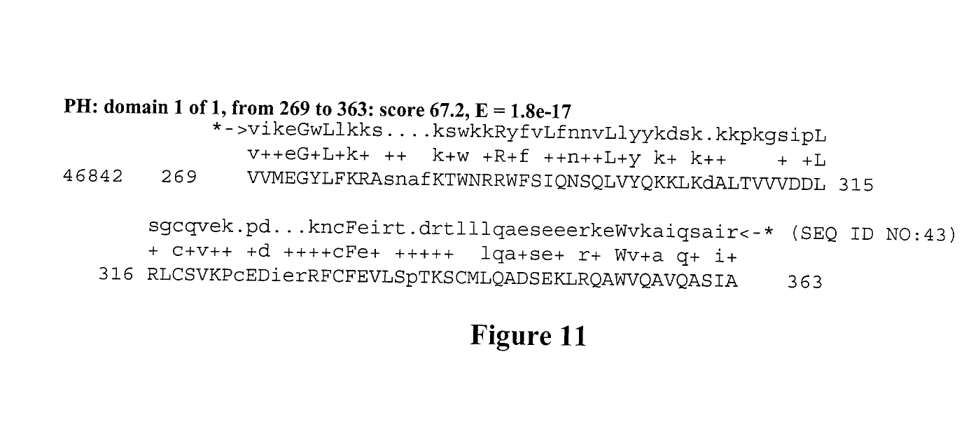

As used herein, the term “PH domain” includes an amino acid sequence of about 80 to 120 amino acid residues in length and having a bit score for the alignment of the sequence to the PH domain (HMM) of at least 30. Preferably, a PH domain includes at least about 80 to 130 amino acids, more preferably about 85 to 120 amino acid residues, or about 90 to 100 amino acids and has a bit score for the alignment of the sequence to the PH domain (HMM) of at least 40, 50, 60 or greater. The PH domain (HMM) has been assigned the PFAM Accession Number PF00169. An alignment of the PH domain (amino acids 269 to 363 of SEQ ID NO:11) of human 46842 with a consensus amino acid sequence (SEQ ID NO:43) derived from a hidden Markov model is depicted in FIG. 11.

In a preferred embodiment 46842 polypeptide or protein has a “PH domain” or a region which includes at least about 80 to 130 more preferably about 85 to 120 or 90 to 100 amino acid residues and has at least about 70%, 80%, 90%, 95%, 98%, 99%, or more homology with a “PH domain,” e.g., the PH domain of human 46842 (e.g., residues 269 to 363 of SEQ ID NO:11). Preferably the PH domain of the 46842 polypeptide includes the tripeptide sequence “RWF”, e.g., the sequence located at about amino acids 289 to 291 of SEQ ID NO:11. Typically, this tripeptide is part of the six characteristic residues of the PH domain motif, defined by the sequence: K-X-[GAST]-X(6, 11)-[RK]-X-R-[ILVFWY]-[ILVFWY]. The PH domain motif has been described in Dowler et al. (2000), Biochem J 351(Pt 1): 19-31, the contents of which are incorporated herein by reference.

A 46842 molecule can further include an ArfGAP domain. The ArfGAP domain differs structurally from other GAP domains, e.g., the SOS GAP domain. A signature feature of the ArfGAP structure is a bound zinc ion, and conserved cysteine residues for coordinating the zinc ion. An Arf GAP domain structure can be modeled based on the structure of the PAPβ protein (Mandiyan et al. (1999) EMBO J. 18:6890). This domain has a three-stranded β-sheet which is surrounded by five α-helices. The PAPβ structural model also contains two ankyrin repeats located on the carboxy terminal side of the ArfGAP. These two ankyrin repeats have an extensive surface which contacts the ArfGAP domain. The ArfGAP domain is highly evolved to bind to Arf small GTPases and to stimulated their ability to hydrolyze GTP to GDP.

A 46842 polypeptide can include an “ArfGAP domain” or regions homologous with an “ArfGAP domain”.

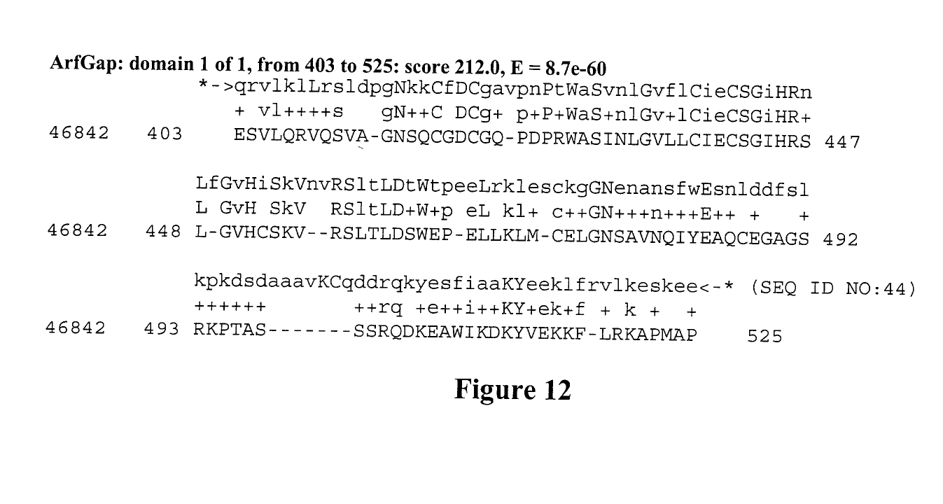

As used herein, the term “ArfGAP domain” includes an amino acid sequence of about 100 to 140 amino acid residues in length and having a bit score for the alignment of the sequence to the ArfGAP domain (HMM) of at least 195. Preferably, an ArfGAP domain includes the motif Cys-X-X-Cys-X(16,17)-Cys-X-X-Cys (SEQ ID NO:62). Preferably, an ArfGAP domain includes at least about 100 to 140 amino acids, more preferably about 110 to 130 amino acid residues, or about 115 to 125 amino acids and has a bit score for the alignment of the sequence to the ArfGAP domain (HMM) of at least 100, 120, 140, 160, 180, preferably 190 or greater. The ArfGAP domain (HMM) has been assigned the PFAM Accession Number PF01412. An alignment of the ArfGAP domain (amino acids 403 to 525 of SEQ ID NO:11) of human 46842 with a consensus amino acid sequence (SEQ ID NO:44) derived from a hidden Markov model (PFAM) is depicted in FIG. 12.

In a preferred embodiment, 46842 polypeptide or protein has a “ArfGAP domain” or a region which includes at least about 100 to 140 more preferably about 110 to 130 or 115 to 125 amino acid residues and has at least about 60%, 70% 80% 90% 95%, 99%, or 100% homology with a “ArfGAP domain,” e.g., the ArfGAP domain of human 46842 (e.g., residues 403 to 525 of SEQ ID NO:11). Preferably, the 46842 protein also includes the sequence matching the ArfGAP zinc ion coordinating motif, defined by the sequence: Cys-X-X-Cys-X(16,17)-Cys-X-X-Cys (SEQ ID NO:62). An ArfGAP zinc ion coordinating motif is present in the human 46842 sequence, located at about amino acid residues 421 to 440 of SEQ ID NO:11.

A 46842 polypeptide can include at least one, preferably two “ankyrin repeats” or regions homologous with “ankyrin repeats”.

Ankyrin repeats are short, approximately 30 to 40 amino acid elements form two α-helices, separated by a β-turn. The repeats are typically, consecutive, allowing the helices to pack against one another to form an accordion like structure. Key features of ankyrin repeats include highly conserved alanine, glycine, and dileucine residues.

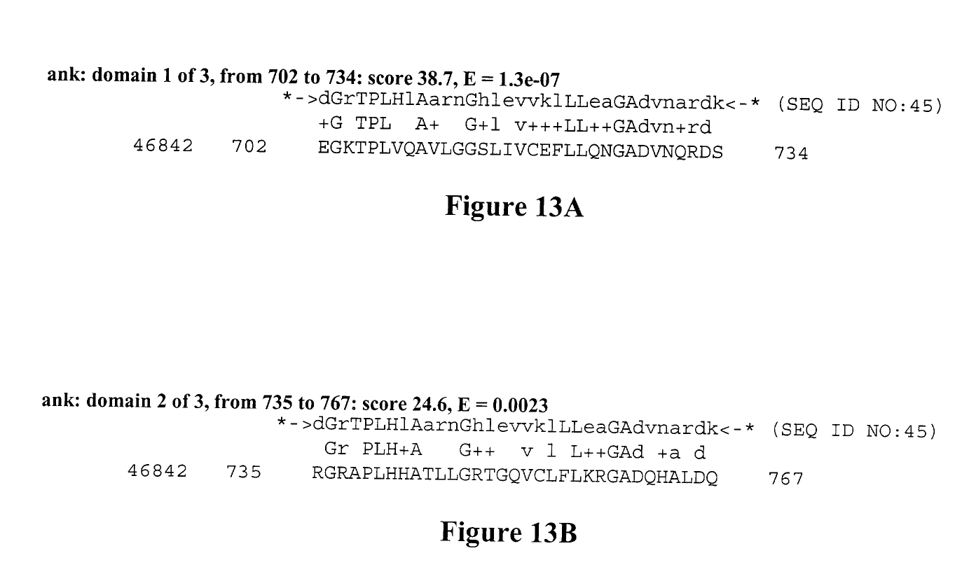

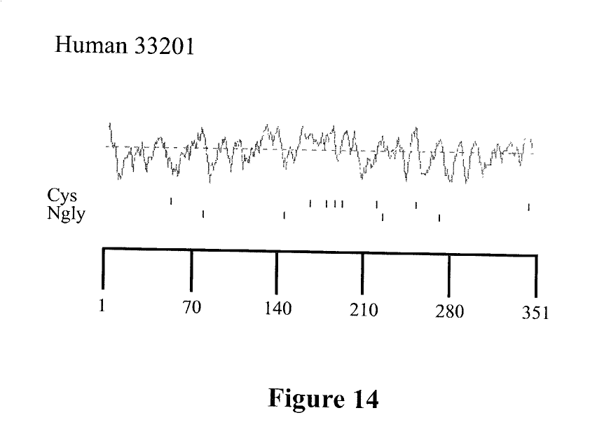

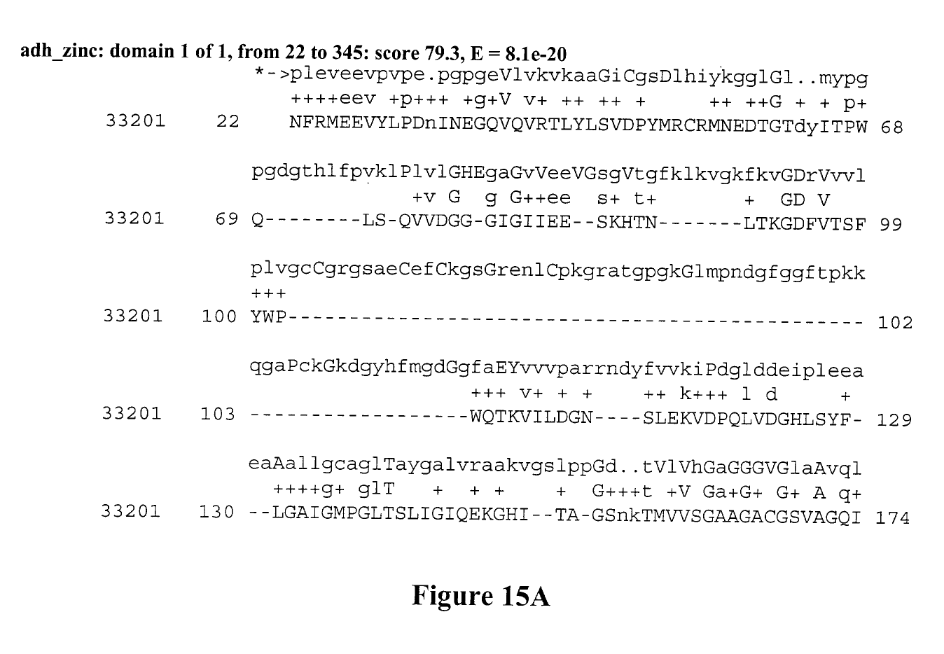

As used herein, the term “ankyrin repeat” includes an amino acid sequence of about 30 to 50 amino acid residues in length and having a bit score for the alignment of the sequence to the ankyrin repeat (HMM) of at least 15. Preferably, a centaurin domain includes at least about 20 to 50 amino acids, more preferably about 25 to 45 amino acid residues, or about 30 to 40 amino acids and has a bit score for the alignment of the sequence to the centaurin domain (HMM) of at least 5, more preferably 10, 15, 16 or greater. The ankyrin repeat domain (HMM) has been assigned the PFAM Accession Number PF00023. An alignment of the ankyrin repeats (amino acids 702 to 734, and 735 to 767 of SEQ ID NO:11) of human 46842 with a consensus amino acid sequence (SEQ ID NO:45) derived from a PFAM hidden Markov model (HMM) is depicted in FIGS. 13A and 13B.

In a preferred embodiment, a 46842 polypeptide or protein has a “ankyrin repeat domain” or a region which includes at least about 20 to 50 more preferably about 25 to 45 or 30 to 40 amino acid residues and has at least about 60%, 70% 80% 90% 95%, 99%, or 100% homology with a “ankyrin repeat domain”, e.g., the ankyrin repeat domains of human 46842 (e.g., residues 702 to 734, and 735 to 767 of SEQ ID NO:11). Preferably, the ankyrin repeats include conserved residues, e.g., the conserved alanine located at about residue 710, glycine at about residue 714, dileucine at about residues 722 to 723, and glycine at about residues 726; the conserved alanine located at about residue 743, glycine at about residue 747, leucine at about residue 756, and glycine at about residues 759 of SEQ ID NO:11.

To identify the presence of a “PH domain”, an “ArfGAP domain”, or an “ankyrin repeat domain” in a 46842 protein sequence, and make the determination that a polypeptide or protein of interest has a particular profile, the amino acid sequence of the protein can be searched against the Pfam database of HMMs (e.g., the Pfam database, release 2.1) using the default parameters. For example, the hmmsf program, which is available as part of the HMMER package of search programs, is a family specific default program for MILPAT0063 and a score of 15 is the default threshold score for determining a hit. Alternatively, the threshold score for determining a hit can be lowered (e.g., to 8 bits). A description of the Pfam database can be found in Sonhammer et al. (1997) Proteins 28(3):405-420 and a detailed description of HMMs can be found, for example, in Gribskov et al.(1990) Meth. Enzymol. 183:146-159; Gribskov et al. (1987) Proc. Natl. Acad. Sci. USA 84:4355-4358; Krogh et al. (1994) J. Mol. Biol. 235:1501-1531; and Stultz et al. (1993) Protein Sci. 2:305-314, the contents of which are incorporated herein by reference. A search was performed against the HMM database resulting in the identification of: a “PH domain” in the amino acid sequence of human 46842 at about residues 269 to 363 of SEQ ID NO:11 (see FIG. 11); an “ArfGAP domain” at about residues 403 to 525 of SEQ ID NO:11 (see FIG. 12); and two “ankyrin repeats” at about residues 702 to 734, and 735 to 767 of SEQ ID NO:11 (see FIGS. 13A and 13B).

Alternatively, to identify the presence of a PH domain, an ArfGAP domain, or an ankyrin repeat in a 46842 protein sequence, and make the determination that a polypeptide or protein of interest has a particular profile, the amino acid sequence of the protein can be also be searched against a SMART database (Simple Modular Architecture Research Tool), of HMMs as described in Schultz et al. (1998), Proc. Natl. Acad. Sci. USA 95:5857 and Schultz et al. (200) Nucl. Acids Res 28:231. The database contains domains identified by profiling with the hidden Markov models of the HMMer2 search program (R. Durbin et al. (1998) Biological sequence analysis: probabilistic models of proteins and nucleic acids. Cambridge University Press). The database also is extensively annotated and monitored by experts to enhance accuracy.

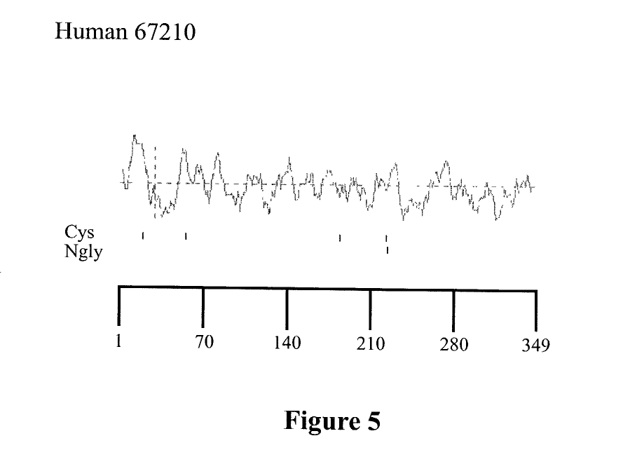

The human 46842 amino acid sequence can also be searched against a database of domains, e.g., the ProDom database (Corpet et al. (1999), Nucl. Acids Res. 27:263-267). For example, FIG. 5 of U.S. S No. 60/250,327 shows a BLAST alignment of the amino terminal domain of human 46842 with a consensus amino acid sequence for oligophrenins derived from a ProDomain No. 1568 (Release 1999.2; see also ProDom family PD023027 (ProDomain Release 2000.1). The ProDom protein domain database consists of an automatic compilation of homologous domains. Current versions of ProDom are built using recursive PSI-BLAST searches (Altschul SF et al. (1997) Nucleic Acids Res. 25:3389-3402; Gouzy et al. (1999) Computers and Chemistry 23:333-340.) of the SWISS-PROT 38 and TREMBL protein databases. The database automatically generates a consensus sequence for each domain. A BLAST search was performed against the ProDom database resulting in the identification of a consensus amino acid sequence for oligophrenins in the amino acid sequence of human 46842 at about residues 3 to 183 of SEQ ID NO:11.

A 46842 family member can include at least one PH domain, at least one ArfGAP domain, and at least one, preferably two ankyrin repeats. Furthermore, a 46842 family member can include: at least one ArfGAP zinc ion coordinating motifs; at least one, two, three, four, five, six, seven, eight, nine, ten, eleven, preferably twelve predicted protein kinase C phosphorylation sites (PS00005); at least one, two, three, four, five, six, seven, eight, nine, ten, eleven, twelve, preferably fourteen predicted casein kinase II phosphorylation sites (PS00006); at least one, two, preferably three predicted cAMP/cGMP-dependent protein kinase phosphorylation sites (PS00004); at least one predicted tyrosine kinase phosphorylation site (PS00007); at least one predicted glycosaminoglycan attachment site (PS00002); and at least one, two, three, four, five, six, seven, eight, nine, preferably ten predicted N-myristylation sites (PS00008).

As the 46842 polypeptides of the invention may modulate 46842-mediated activities, they may be useful as of for developing novel diagnostic and therapeutic agents for 46842-mediated or related disorders, as described below.

As used herein, a “46842 activity”, “biological activity of 46842” or “functional activity of 46842”, refers to an activity exerted by a 46842 protein, polypeptide or nucleic acid molecule on e.g., a 46842-responsive cell or on a 46842 substrate, e.g., a protein substrate, as determined in vivo or in vitro. In one embodiment, a 46842 activity is a direct activity, such as an association with a 46842 target molecule. A “target molecule” or “binding partner” is a molecule with which a 46842 protein binds or interacts in nature. In an exemplary embodiment, a 46842 polypeptide or protein is a GTP activating protein (GAP) for Arf proteins, e.g., a phosphoinositide-activated GAP protein.

A 46842 activity can also be an indirect activity, e.g., a cellular signaling activity mediated by another protein that interacts with a 46842 protein, e.g., an Arf or Arf-like protein. Based on the above-described sequence similarities, the 46842 molecules of the present invention are predicted to have similar biological activities as centaurin family members. For example, the 46842 proteins of the present invention can have one or more of the following activities: (1) Arf protein binding; (2) stimulation of Arf GTP hydrolysis; (3) phosphoinositide binding; (4) membrane binding; (5) binding and regulation of actin cytoskeletal structures, e.g., cortical actin, focal adhesions, and/or membrane ruffles; (6) modulation of vesicular transport, e.g., protein trafficing and secretion; or (7) the ability to antagonize or inhibit, competitively or non-competitively, any of 1-6.

In addition, the 46842 molecules of the invention can be expected to function in tissues in which they are expressed. Thus, the 46842 molecules can act as novel diagnostic targets and therapeutic agents for controlling cell motility and adhesion disorders (e.g., a metastatic disorder or an immunological disorder, e.g., a disorder related to an inability to clear an infection, e.g., a bacterial or viral infection), and secretory disorders (e.g., a neurological or viral disorder).

33201

The 33201 protein contains a significant number of structural characteristics in common with members of the dehydrogenase/reductase family. The term “family” when referring to the protein and nucleic acid molecules of the invention means two or more proteins or nucleic acid molecules having a common structural domain or motif and having sufficient amino acid or nucleotide sequence homology as defined herein. Such family members can be naturally or non-naturally occurring and can be from either the same or different species. For example, a family can contain a first protein of human origin as well as other distinct proteins of human origin, or alternatively, can contain homologues of non-human origin, e.g., rat or mouse proteins. Members of a family can also have common functional characteristics.

A dehydrogenase/reductase family member is an enzyme composed of two subunits. Members of the dehydrogenase/reductase family include alcohol dehydrogenase and quinone reductase. The α, β, and γ forms of these subunits can be mixed and matched among the different human forms of the dehydrogenases, creating mixed dimers that are still active. These dimers can exist as homo- or hetero-dimers. Dehydrogenase/reductases typically use two interactions to accomplish their reaction with ethanol. For example, the first is to use an associated zinc atom to position the alcoholic group on ethanol. The second is to use an NAD cofactor (from the vitamin niacin), which performs the reaction of oxidizing the alcohol.

Thus, a 33201 polypeptide can include a domain having dehydrogenase or a reductase activity. Furthermore, a 33201 polypeptide can have domain(s) that confer both dehydrogenase and reductase activity. The particular activity of such a polypeptide, i.e., whether it functions as a dehydrogenase or a reductase may depend upon the conditions, e.g., coenzyme availability, etc. Because of the reversibility of the reaction, the dehydrogenase and reductase domains of a 33201 polypeptide may be the same. Alternatively, the proteins may be bi-functional in that two separate domains confer dehydrogenase and reductase activity. The domains that confer these activities may therefore be located in the same or different regions of the polypeptide. Similarly, subsequences or fragments of 33201 can be capable of one of either of the activities, or can be capable of both dehydrogenase and reductase activity.

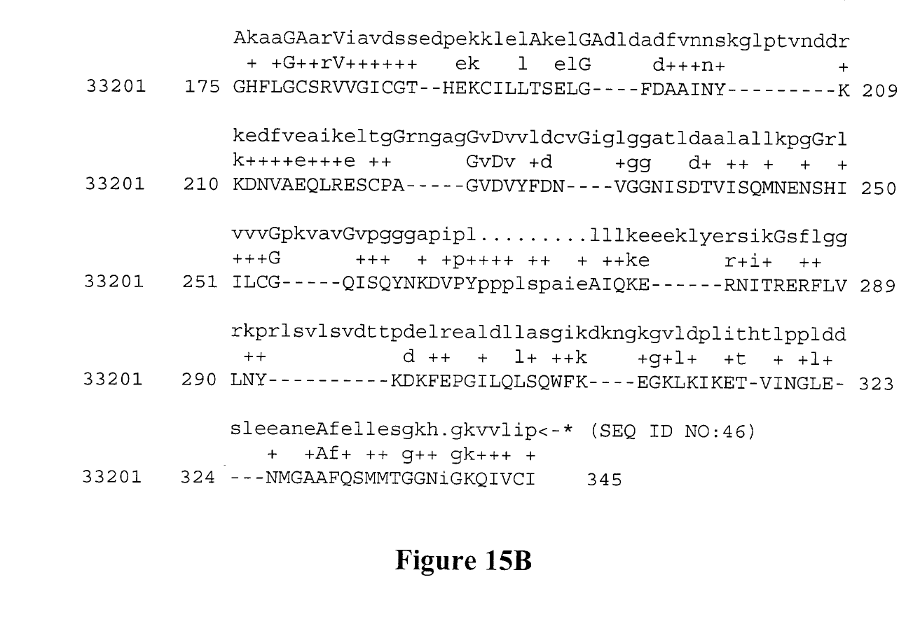

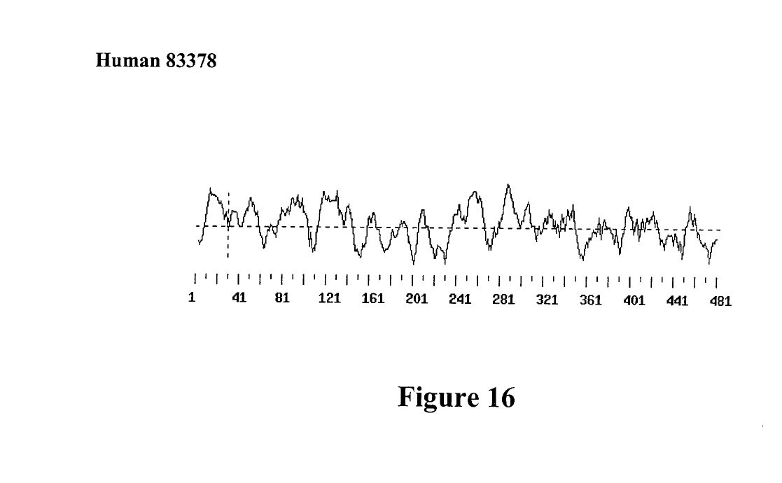

A 33201 polypeptide can include a “dehydrogenase/reductase domain” or an “alcohol dehydrogenase domain” or regions homologous with a “dehydrogenase/reductase domain”. Dehydrogenase/reductase domains typically participate in the reversible oxidation of alcohols to aldehydes. As used herein, the term “dehydrogenase/reductase domain” includes an amino acid sequence of about 200 to 400 amino acid residues in length and having a bit score for the alignment of the sequence to the dehydrogenase/reductase domain (HMM) of at least 40. Preferably, an dehydrogenase/reductase domain includes at least about 250-375 amino acids, more preferably about 275-350 amino acid residues, or about 310-340 amino acids and has a bit score for the alignment of the sequence to the dehydrogenase/reductase domain (HMM) of at least 50, 60, 70 or greater. The dehydrogenase/reductase domain (HMM) has been assigned the PFAM Accession Number PF00107. An alignment of the dehydrogenase/reductase domain (amino acids 22 to 345 of SEQ ID NO:14) of human 33201 with a consensus amino acid sequence derived from a hidden Markov model is depicted in FIG. 15. In one embodiment, a 33201 polypeptide is a zinc-containing dehydrogenase/reductase, e.g., is capable of binding one or two atoms of zinc.

When members of the superfamily are aligned, several amino acid residues are highly conserved among the various proteins. For example, a comparison of 106 proteins belonging to this superfamily reveals that only three residues are strictly conserved among all members (corresponding to Gly66, Gly86, and Gly201 of mammalian class I dehydrogenase/reductase) (Persson et al. (1994) Eur J. Biochem. 226:15-22). In preferred embodiments, a 33201 polypeptide or protein includes at least three conserved glycine residues, e.g., the three conserved glycine residues of human 33201 located at about amino acid residues 76, 93, and 224 of SEQ ID NO:14.

In a preferred embodiment 33201 polypeptide or protein has a “alcohol hydrogenase domain” or a region which includes at least about 250 to 375, more preferably about 275 to 350, or 310 to 340 amino acid residues and has at least about 50%, 60%, 70%, 80%, 90%, 95%, 98%, 99%, or more homology with a “dehydrogenase/reductase domain,” e.g., the dehydrogenase/reductase domain of human 33201 (e.g., residues 22 to 345 of SEQ ID NO:14).

To identify the presence of an “dehydrogenase/reductase” domain in a 33201 protein sequence, and make the determination that a polypeptide or protein of interest has a particular profile, the amino acid sequence of the protein can be searched against the Pfam database of HMMs (e.g., the Pfam database, release 2.1) using the default parameters. For example, the hmmsf program, which is available as part of the HMMER package of search programs, is a family specific default program for MILPAT0063 and a score of 15 is the default threshold score for determining a hit. Alternatively, the threshold score for determining a hit can be lowered (e.g., to 8 bits). A description of the Pfam database can be found in Sonhammer et al. (1997) Proteins 28(3):405-420 and a detailed description of HMMs can be found, for example, in Gribskov et al. (1990) Meth. Enzymol. 183:146-159; Gribskov et al. (1987) Proc. Natl. Acad. Sci. USA 84:4355-4358; Krogh et al. (1994) J. Mol. Biol. 235:1501-1531; and Stultz et al. (1993) Protein Sci. 2:305-314, the contents of which are incorporated herein by reference. A search was performed against the HMM database resulting in the identification of an “dehydrogenase/reductase” domain in the amino acid sequence of human 33201 at about residues 22 to 345 of SEQ ID NO:14 (see FIG. 15).

A 33201 family member can include at least one dehydrogenase/reductase domain. Furthermore, a 33201 family member can include: at least one, two, preferably three conserved glycine residues; at least one, two, three, preferably four-predicted N-glycosylation sites (PS00001); at least one predicted protein kinase C phosphorylation sites (PS00005); at least one, preferably two predicted casein kinase II phosphorylation sites (PS00006); and at least one, two, three, four, five, six, seven, preferably eight predicted N-myristylation sites (PS00008).

As the 33201 polypeptides of the invention may modulate 33201-mediated activities, they may be useful as of for developing novel diagnostic and therapeutic agents for 33201-mediated or related disorders, as described below.

As used herein, a “33201 activity”, “biological activity of 33201” or “functional activity of 33201”, refers to an activity exerted by a 33201 protein, polypeptide or nucleic acid molecule. For example, a 33201 activity can be an activity exerted by 33201 in a physiological milieu on, e.g., a 33201-responsive cell or on a 33201 substrate, e.g., a protein substrate. A 33201 activity can be determined in vivo or in vitro. In one embodiment, a 33201 activity is a direct activity, such as an association with a 33201 target molecule. A “target molecule” or “binding partner” is a molecule with which a 33201 protein binds or interacts in nature, e.g., an alcohol or a quinone. In an exemplary embodiment, a 33201 polypeptide or protein is an enzyme, e.g., an enzyme that catalyzes the oxidation and/or reduction of a 33201 substrate, e.g., a molecule that contains an alcohol group or a quinone.

A 33201 activity can also be an indirect activity, e.g., a cellular signaling activity mediated by a 33201 substrate, such that the activity of the 33201 upon the substrate alters its ability to signal, e.g., alters its concentration.

Depending on the conditions, such as cofactor and/or coenzyme availability, a 33201 polypeptide can have reductase or dehydrogenase activity. As used herein, the term “reductase activity” means the ability to add one or more hydrides to a substrate having, for example, a keto group. Typically, the hydride is provided by NADH, NADP, NADPH, or other coenzyme or hydride donor. For example, in the biological conversion of 4-androstenedione to testosterone, a hydrogen ion is transferred from NADPH to the substrate thereby forming NADP+ product. Coenzymes of 33201 polypeptide also include, but are not limited to NAD+ and NAD+ analogues (Plapp et al. (1986) Biochemistry 25:5396-5402 and Yamazaki et al. (1984) J. Biochem. 95:109-115), NADH, NADP+, and NADPH (LaRhee et al. (1984) Biochemistry 23:486-491 and Pollow et al. (1976) J. Steroid Biochem. 7:45-50). For example, a 33201 polypeptide can catalyze the reduction of quinone.

A 33201 polypeptide can have a “dehydrogenase activity.” As used herein, the term “dehydrogenase activity” means the ability to directly or indirectly remove a hydride from a substrate. Typically, after removal of a hydride from a substrate, electrons of the hydride are transferred to NAD+, NADP+, or other coenzyme (e.g., 3-acetylpyridine adenine dinucleotide phosphate) or hydride acceptor. For example, if the substrate has hydroxyl, dehydrogenation converts the hydroxyl to a keto group and produces NADH or NADPH and a proton. Hydride removal from substrate however does not require the presence of an acceptor. Free hydride can be detected optically by H+ binding to a dye molecule, for example

Based on the above-described sequence similarities, the 33201 molecules of the present invention are predicted to have one or more biological activities of dehydrogenase/reductase family members. For example, the 33201 proteins of the present invention can have one or more of the following activities: (1) the ability to metabolize an alcohol, e.g., to catalyze the reversible oxidation of ethanol to acetaldehyde; (2) the ability to metabolize or remove endogenous or non-endogenous (e.g., xenobiotic) substances, such as toxins; (3) the ability to catalyze the oxidation of retinoic acid; (4) the ability to catalyze the reduction of a substrate, e.g., quinone; (5) the ability to modulate dopamine metabolism; (6) the ability to reduce a bioreductive compound, e.g., a bioreductive antitumor quinone; (7) the ability to modulate cellular differentiation; (8) the ability to modulate cellular proliferation; or (9) the ability to agonize or antagonize one or more of the activities 1-7.

Consequently, the 33201 molecules of the invention can be involved in pathological conditions, e.g., conditions involving cellular degeneration (e.g., neurodegeneration). Furthermore, the 33201 molecules of the invention can be allelic variants associated with a genetic disorder or predisposition due to decreased or increased activity/expression of the variant relative to wild-type activity, e.g., increased predisposition towards alcoholism or increased ethanol sensitivity due to a mutation in an dehydrogenase/reductase gene that correlates with predisposition/sensitivity or modulation of chemosensitivity, e.g., due to increased or decreased quinone reductase activity of the tissue relative to wild type activity.

In addition, the 33201 molecules of the invention can be expected to function in tissues in which they are expressed. Thus, the 33201 molecules can act as novel diagnostic targets and therapeutic agents for controlling one or more disorders, including metabolic disorders, liver disorders, kidney disorders, digestive disorders, and cellular proliferative and/or differentiative disorders.

83378, 84233, 64708, 85041, and 84234

The 83378, 84233, 64708, 85041, and 84234 proteins contain a significant number of structural characteristics in common with members of the metal transporter family. The term “family” when referring to the protein and nucleic acid molecules of the invention means two or more proteins or nucleic acid molecules having a common structural domain or motif and having sufficient amino acid or nucleotide sequence homology as defined herein. Such family members can be naturally or non-naturally occurring and can be from either the same or different species. For example, a family can contain a first protein of human origin as well as other distinct proteins of human origin, or alternatively, can contain homologues of non-human origin, e.g., rat or mouse proteins. Members of a family can also have common functional characteristics.

Members of the metal transporter family of proteins are membrane proteins that increase cellular tolerance to divalent metal ions such as zinc, cadmium, and cobalt by mediating cation diffusion across membranes. Some metal transporter proteins are efflux pumps that remove divalent metal ions from cells. Other metal transporter proteins function to increase cellular tolerance to metal ions by mediating the sequestration of ions in subcellular compartments. Some metal transporter proteins are characterized by a topology comprising six membrane spanning domains, a histidine-rich loop between the fourth and fifth membrane spanning domains, and a long C-terminal tail. Examples of metal transporter proteins include ZnT-1, ZnT-2, and ZnT-3. ZnT-1, a plasma membrane protein, functions as a zinc transporter, mediating the cellular efflux of zinc. ZnT-2 is located in vesicles within a cell and mediates the vesicular sequestration of zinc. ZnT-3 is thought to participate in the accumulation of zinc in synaptic vesicles. As the 83378, 84233, 64708, 85041, or 84234 proteins show homology to metal transporter proteins, they are likely to be involved in mediating tolerance to divalent metal ions, e.g., zinc, in the cells in which they are expressed. 83378, 84233, 64708, 85041, or 84234 proteins are predicted to be, like other members of the metal transporter family, transmembrane proteins, embedded either within the plasma membrane or the membrane of a subcellular organelle.

A 83378, 84233, 64708, 85041, or 84234 polypeptide can include a “cation efflux domain” or regions homologous with a “cation efflux domain.”

As used herein, the term “cation efflux domain” includes an amino acid sequence of about 100 to 400 amino acid residues in length and having a bit score for the alignment of the sequence to the cation efflux domain (HMM) of at least 50. Preferably, a cation efflux domain includes at least about 150 to 350 amino acids, more preferably about 200 to 350 amino acid residues, or about 220 to 330 amino acids and has a bit score for the alignment of the sequence to the cation efflux domain (HMM) of at least 65 or greater. The cation efflux domain (HMM) has been assigned the PFAM Accession Number PF01545.

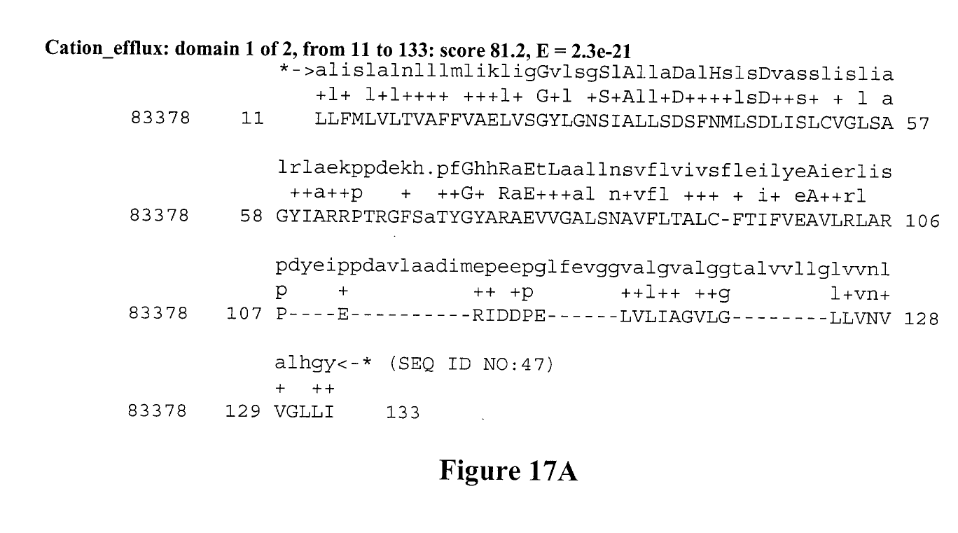

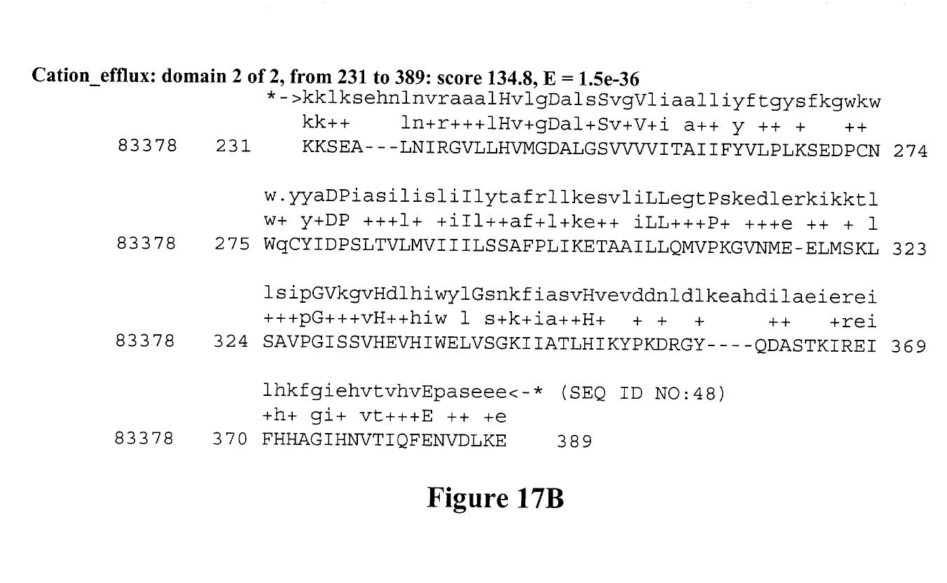

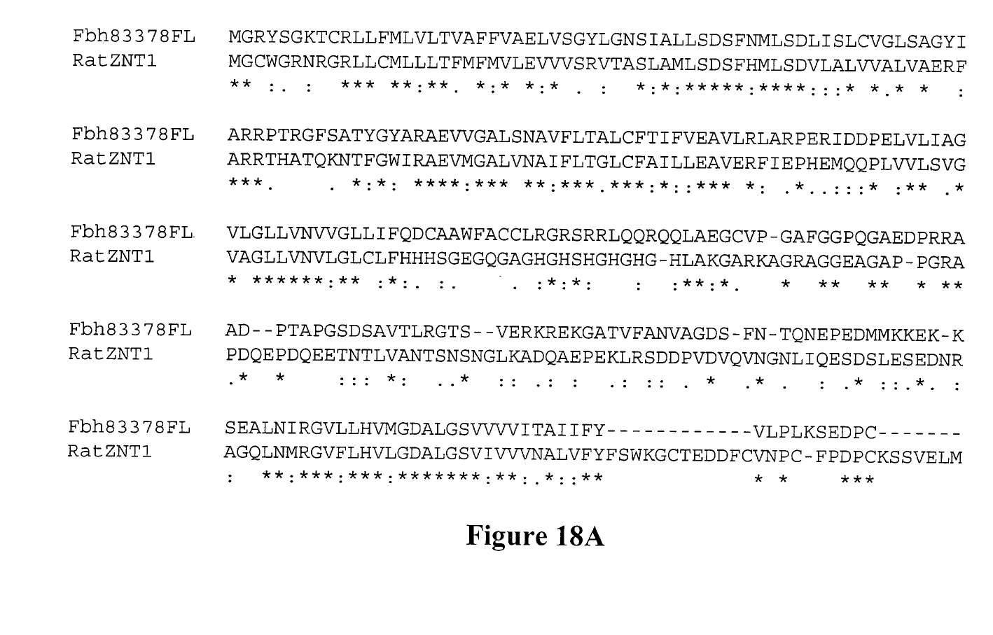

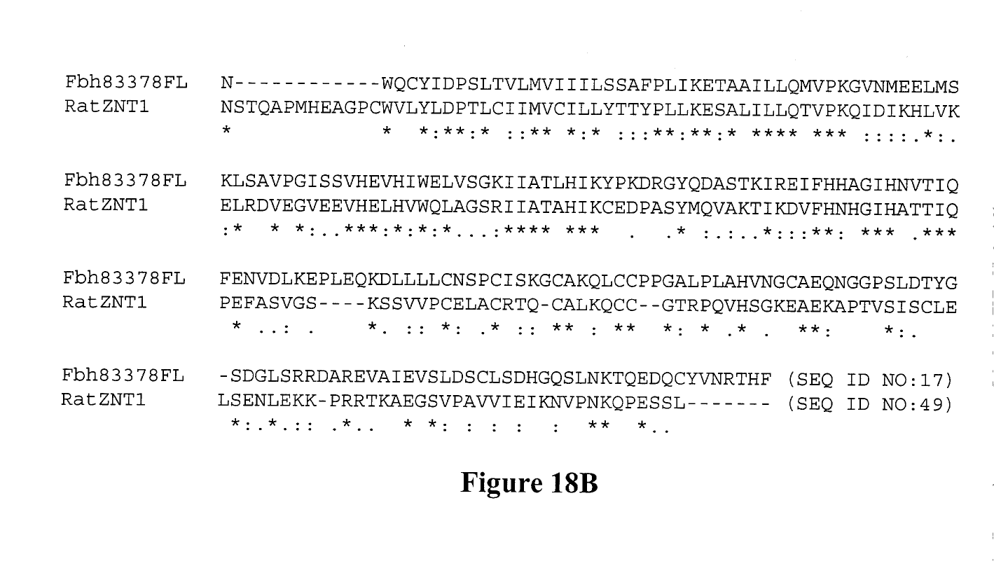

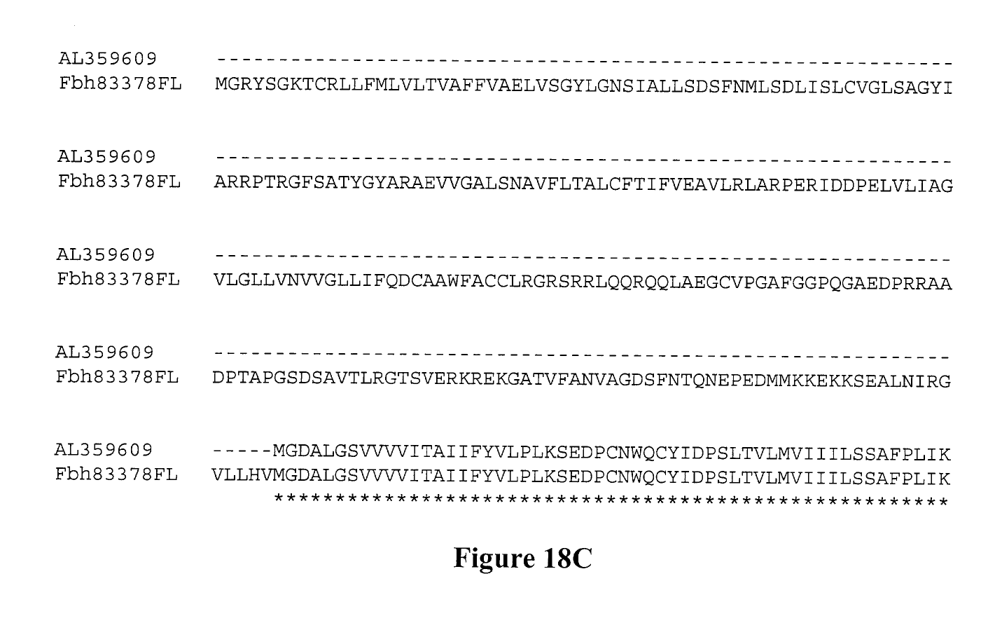

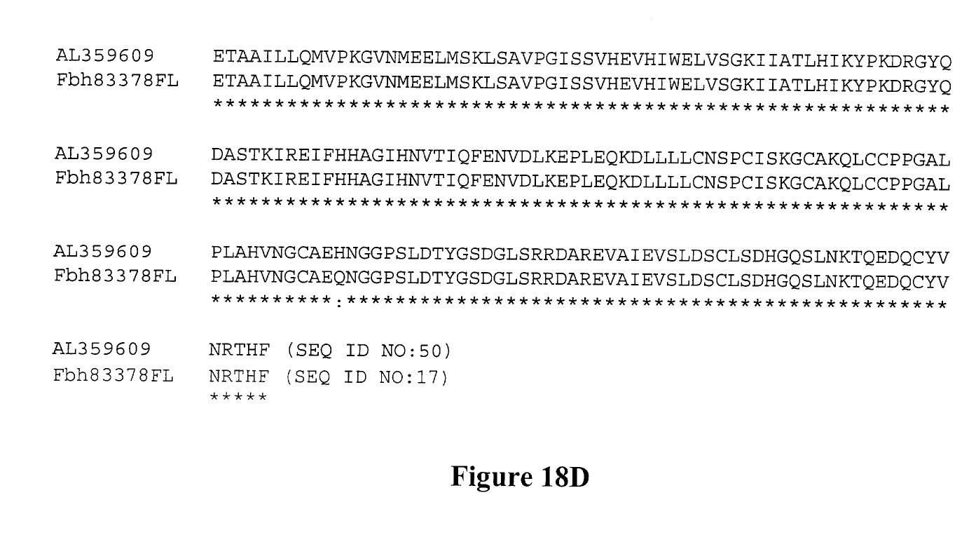

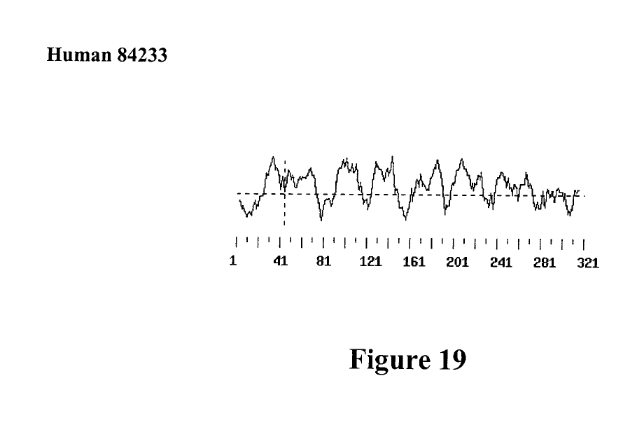

Alignments of the first and second cation efflux domains (amino 11 to 133 and 231 to 389 of SEQ ID NO:17) of human 83378 with consensus amino acid sequences (SEQ ID NO:47 and SEQ ID NO:48) derived from hidden Markov models are depicted in FIGS. 17A and 17B. A cation efflux domain of a 83378 polypeptide preferably includes about 120-160 amino acids and has a bit score for the alignment of the sequence to the cation efflux domain (HMM) of at least 80, 130, or greater.

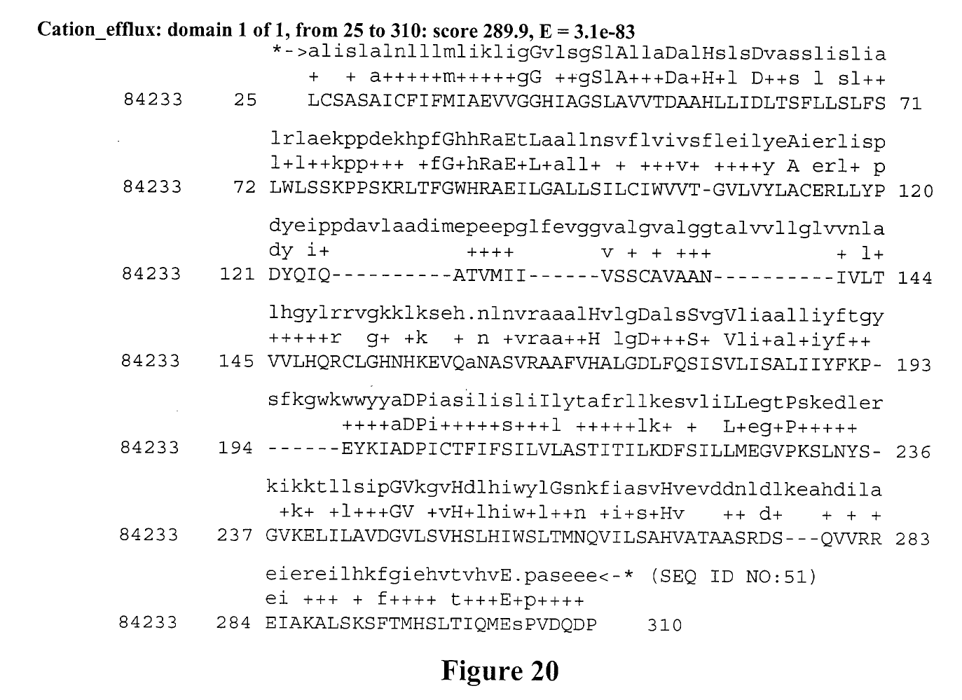

An alignment of the cation efflux domain (amino acids 25 to 310 of SEQ ID NO:20) of human 84233 with a consensus amino acid sequence (SEQ ID NO:51) derived from a hidden Markov model is depicted in FIG. 20. A cation efflux domain of a 84233 polypeptide preferably includes about 270 to 290 amino acids and has a bit score for the alignment of the sequence to the cation efflux domain (HMM) of at least 250 or greater.

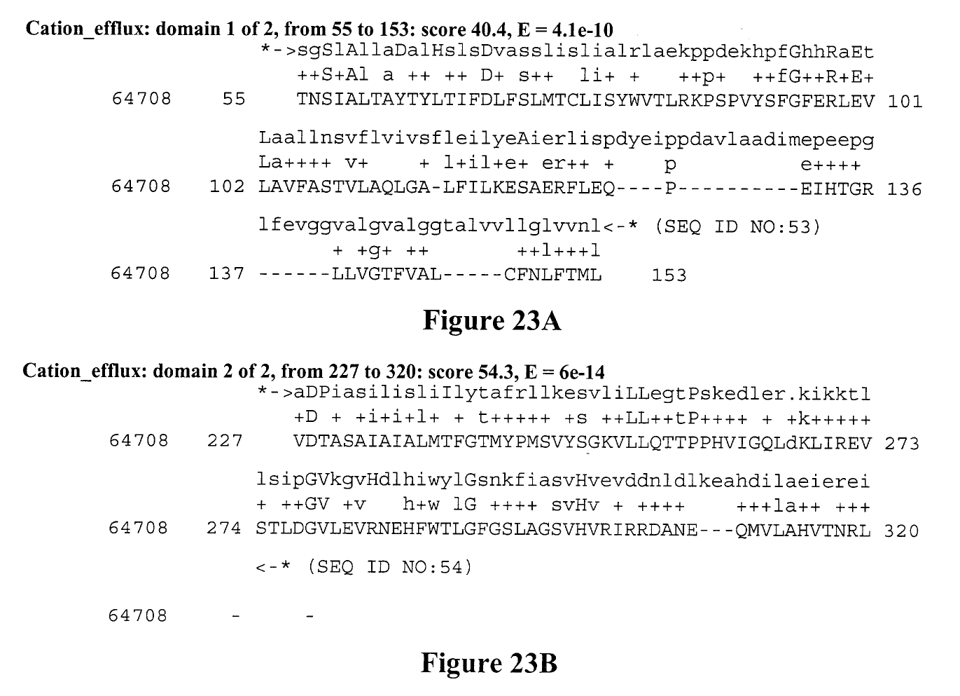

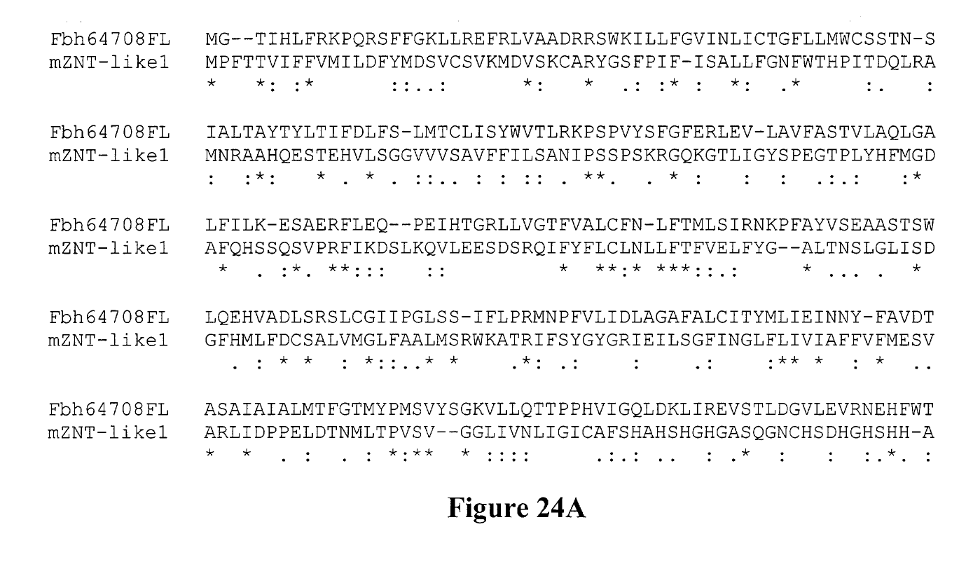

Alignments of the first and second cation efflux domains (amino acids 55 to 153 and 227 to 320 of SEQ ID NO:23) of human 64708 with consensus amino acid sequences (SEQ ID NO:53 and SEQ ID NO:54) derived from hidden Markov models are depicted in FIGS. 23A and 23B. A cation efflux domain of a 64708 polypeptide preferably includes about 90 to 100 amino acids and has a bit score for the alignment of the sequence to the cation efflux domain (HMM) of at least 40 or greater.

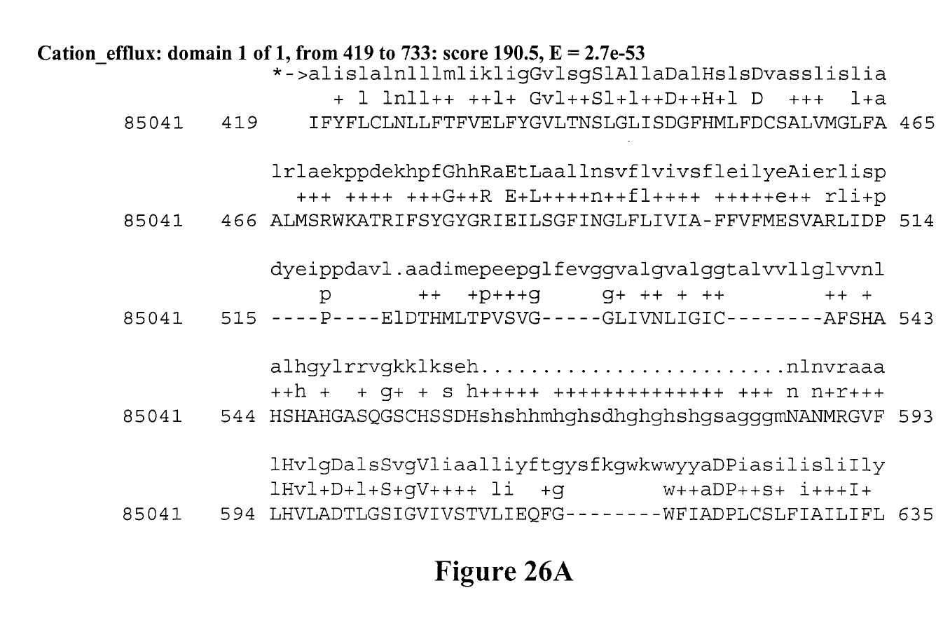

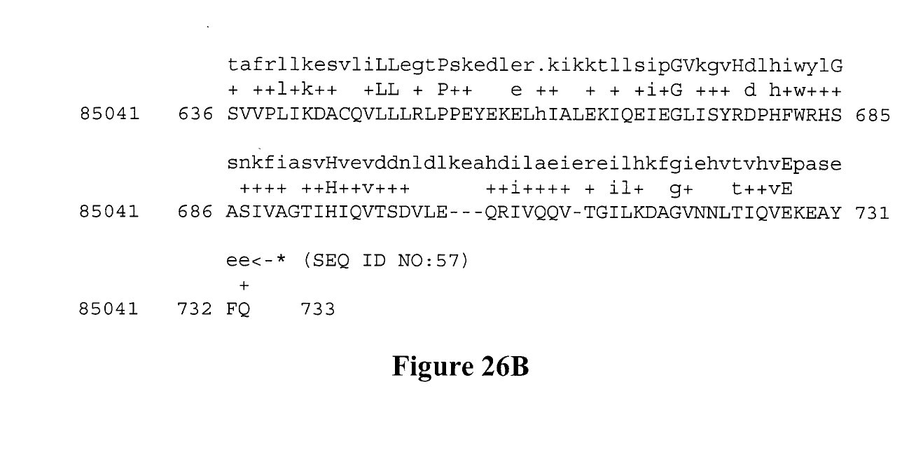

An alignment of the cation efflux domain (amino acids 419 to 733 of SEQ ID NO:26) of human 85041 with a consensus amino acid sequence (SEQ ID NO:57) derived from a hidden Markov model is depicted in FIG. 26. A cation efflux domain of a 85041 polypeptide preferably includes about 310 to 320 amino acids and has a bit score for the alignment of the sequence to the cation efflux domain (HMM) of at least 180 or greater.

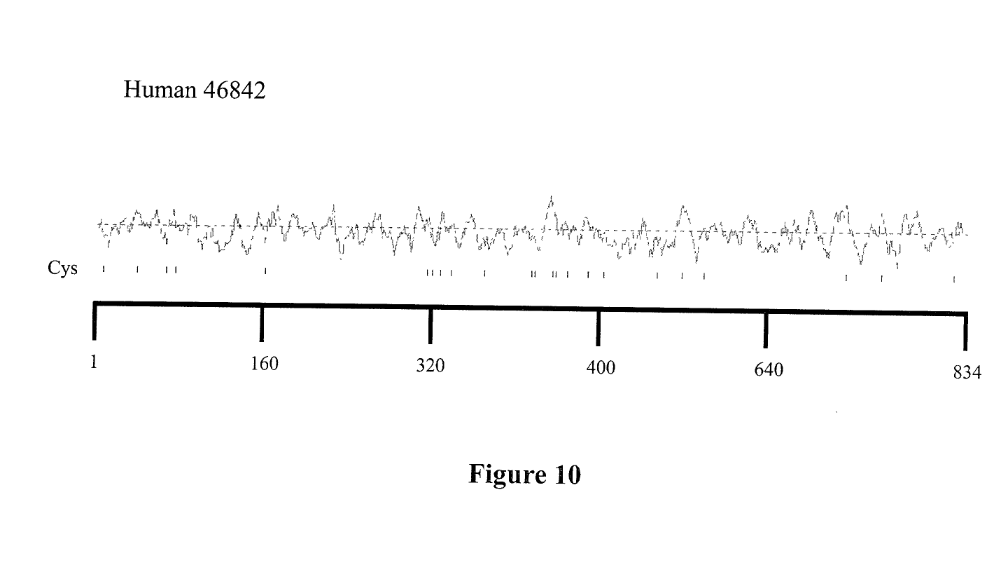

An alignment of the cation efflux domain (amino acids 38 to 349 of SEQ ID NO:29) of human 84234 with a consensus amino acid sequence (SEQ ID NO:58) derived from a hidden Markov model is depicted in FIG. 10. A cation efflux domain of a 84234 polypeptide preferably includes about 310 to 320 amino acids and has a bit score for the alignment of the sequence to the cation efflux domain (HMM) of at least 160 or greater.

In a preferred embodiment 83378, 84233, 64708, 85041, or 84234 polypeptide or protein has a “cation efflux domain” or a region which includes at least about 150 to 350 more preferably about 200 to 350 or 220 to 330 amino acid residues and has at least about 50%, 60%, 70% 80% 90% 95%, 99%, or 100% homology with a “cation efflux domain,” e.g., the cation efflux domain of human 83378, 84233, 64708, 85041, or 84234 (e.g., residues 11 to 133 or 231 to 389 of SEQ ID NO:17, residues 25 to 310 of SEQ ID NO:20, residues 55 to 153 or 227 to 320 of SEQ ID NO:23, residues 419 to 733 of SEQ ID NO:26, or residues 38 to 349 of SEQ ID NO:29).

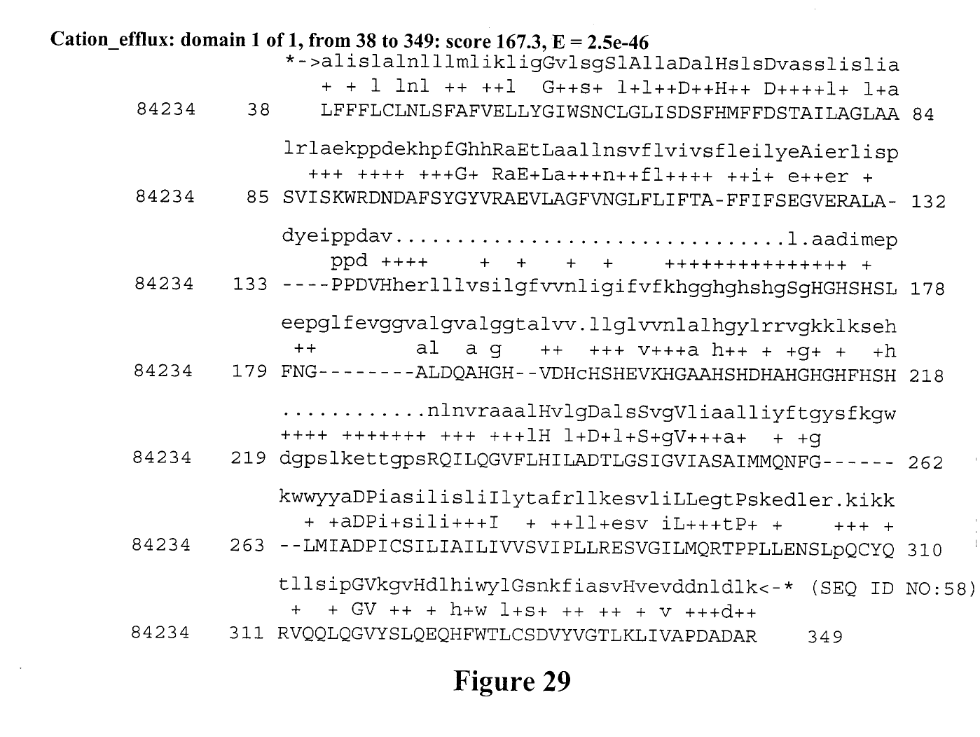

To identify the presence of a “cation efflux” domain in a 83378, 84233, 64708, 85041, or 84234 protein sequence, and make the determination that a polypeptide or protein of interest has a particular profile, the amino acid sequence of the protein can be searched against the Pfam database of HMMs (e.g., the PFAM database, release 2.1) using the default parameters. For example, the hmmsf program, which is available as part of the HMMER package of search programs, is a family specific default program for MILPAT0063 and a score of 15 is the default threshold score for determining a hit. Alternatively, the threshold score for determining a hit can be lowered (e.g., to 8 bits). A description of the Pfam database can be found in Sonhammer et al. (1997) Proteins 28(3):405-420 and a detailed description of HMMs can be found, for example, in Gribskov et al. (990) Meth. Enzymol. 183:146-159; Gribskov et al. (987) Proc. Natl. Acad. Sci. USA 84:4355-4358; Krogh et al. (1994) J. Mol. Biol. 235:1501-1531; and Stultz et al. (1993) Protein Sci. 2:305-314, the contents of which are incorporated herein by reference. Searches were performed against the HMM database resulting in the identification of “cation efflux domains” in the amino acid sequence of human 83378, 84233, 64708, 85041, an 84234 at about: residues 11 to 133 and 231 to 389 of SEQ ID NO:17 (see FIGS. 17A and 17B); residues 25 to 310 of SEQ ID NO:20 (see FIG. 20); residues 55 to 153 and 227 to 320 of SEQ ID NO:23 (see FIGS. 23A and 23B); residues 419 to 733 of SEQ ID NO:26 (see FIG. 26), and residues 38 to 349 of SEQ ID NO:29 (see FIG. 29).

A 83378, 84233, 64708, 85041, or 84234 polypeptide can include a “transmembrane domain” or regions homologous with a “transmembrane domain”.

As used herein, the term “transmembrane domain” includes an amino acid sequence of about 15 amino acid residues in length which spans a phospholipid membrane. More preferably, a transmembrane domain includes about at least 20, 25, 30, 35, 40, or 45 amino acid residues and spans a phospholipid membrane. Transmembrane domains are rich in hydrophobic residues, and typically have an alpha-helical structure. In a preferred embodiment, at least 50%, 60%, 70%, 80%, 90%, 95% or more of the amino acids of a transmembrane domain are hydrophobic, e.g., leucines, isoleucines, tyrosines, or tryptophans. Transmembrane domains are described in, for example, Zagotta W. N. et al., (1996) Annual Rev. Neurosci. 19: 235-263, the contents of which are incorporated herein by reference.

Amino acid residues 11-31, 44-61, 79-98, 115-134, 241-265, and 283-299 of the 83378 protein (SEQ ID NO:17) are predicted to comprise transmembrane domains. Accordingly, 83378 proteins having at least 50-60% homology, preferably about 60-70%, more preferably about 70-80%, or about 80-90% homology with a transmembrane domain of human 83378 are within the scope of the invention.

Amino acid residues 25-49, 58-74, 92-113, 128-147, 167-191, and 201-218 of the 84233 protein (SEQ ID NO:20) are predicted to comprise transmembrane domains. Accordingly, 84233 proteins having at least 50-60% homology, preferably about 60-70%, more preferably about 70-80%, or about 80-90% homology with a transmembrane domain of human 84233 are within the scope of the invention.

Amino acid residues 34-51, 58-82, 101-119, 137-155, 202-219, and 232-249 of the 64708 protein (SEQ ID NO:23) are predicted to comprise transmembrane domains. Accordingly, 64708 proteins having at least 50-60% homology, preferably about 60-70%, more preferably about 70-80%, or about 80-90% homology with a transmembrane domain of human 64708 are within the scope of the invention.

Amino acid residues 59-77, 99-119, 129-145, 152-168, 190-214, 239-258, 267-288, 304-320, 343-362, 419-439, 486-505, 521-541, 592-613, and 618-641 of the 85041 protein (SEQ ID NO:26) are predicted to comprise transmembrane domains. Accordingly, 85041 proteins having at least 50-60% homology, preferably about 60-70%, more preferably about 70-80%, or about 80-90% homology with a transmembrane domain of human 85041 are within the scope of the invention.

Amino acid residues 38-58, 71-87, 105-123, 141-159, 237-256, and 263-286 of the 84234 protein (SEQ ID NO:29) are predicted to comprise transmembrane domains. Accordingly, 84234 proteins having at least 50-60% homology, preferably about 60-70%, more preferably about 70-80%, or about 80-90% homology with a transmembrane domain of human 84234 are within the scope of the invention.

In one embodiment, a 83378, 84233, 64708, 85041, or 84234 protein includes at least one cytoplasmic domain. When located at the N-terminal domain the cytoplasmic domain is referred to herein as an “N-terminal cytoplasmic domain”. As used herein, an “N-terminal cytoplasmic domain” includes an amino acid sequence having about 1-300, preferably about 1-250, preferably about 1-200, more preferably about 1-150, more preferably about 1-100, more preferably about 1-80, or even more preferably about 1-60 amino acid residues in length and is located inside of a cell or intracellularly. The C-terminal amino acid residue of a “N-terminal cytoplasmic domain” is adjacent to an N-terminal amino acid residue of a transmembrane domain in a 83378, 84233, 64708, 85041, or 84234 protein. For example, an N-terminal cytoplasmic domain is located at about amino acid residues 1-10 of SEQ ID NO:17, 1-24 of SEQ ID NO:20, 1-33 of SEQ ID NO:23, 1-58 of SEQ ID NO:26, and 1-37 of SEQ ID NO:29.

In a preferred embodiment, a 83378, 84233, 64708, 85041, or 84234 polypeptide or protein has at least one cytoplasmic domain or a region which includes at least about 5, preferably about 10-300, and more preferably about 30-220 amino acid residues and has at least about 60%, 70%, 80%, 90%, 95%, 99%, or 100% homology with an “cytoplasmic domain,” e.g., at least one cytoplasmic domain of human 83378, 84233, 64708, 85041, or 84234 (e.g., residues 1-10, 62-78, 135-240, and 300-485 of SEQ ID NO:17; residues 1-24, 75-91, 148-166, and 219-320 of SEQ ID NO:20; residues 1-33, 83-100, 156-201, and 250-461 of SEQ ID NO:23; residues 1-58, 120-128, 169-189, 259-266, 321-342, 438-485, 542-591, and 642-765 of SEQ ID NO:26; and residues 1-37, 88-104, 160-236, and 287-376 of SEQ ID NO:29).

In another embodiment, a 52906, 33408, or 12189 protein includes at least one non-cytoplasmic loop. As used herein, the term “loop” includes an amino acid sequence that resides outside of a phospholipid membrane, having a length of at least about 4, preferably about 5-80, and more preferably about 5-60 amino acid residues, and has an amino acid sequence that connects two transmembrane domains within a protein or polypeptide. Non-cytoplasmic loops include extracellular domains (i.e., outside of the cell) and intracellular domains (i.e., within the cell). When referring to membrane-bound proteins found in intracellular organelles (e.g., mitochondria, endoplasmic reticulum, peroxisomes microsomes, vesicles, endosomes, and lysosomes), non-cytoplasmic loops include those domains of the protein that reside in the lumen of the organelle or the matrix or the intermembrane space. Accordingly, the N-terminal amino acid of a non-cytoplasmic loop is adjacent to a C-terminal amino acid of a transmembrane domain in a 83378, 84233, 64708, 85041, or 84234 molecule, and the C-terminal amino acid of a non-cytoplasmic loop is adjacent to an N-terminal amino acid of a transmembrane domain in a 83378, 84233, 64708, 85041, or 84234 molecule. As used herein, a “non-cytoplasmic loop” includes an amino acid sequence located outside of a cell or within an intracellular organelle. For example, a “non-cytoplasmic loop” can be found at about amino acids 32-43, 99-114, and 266-282 of SEQ ID NO:17; at about amino acids 50-57, 114-127, and 192-200 of SEQ ID NO:20; at about amino acids 52-57, 120-136, and 220-231 of SEQ ID NO:23; at about amino acids 78-98, 146-151, 215-238, 289-303, 363-418, 506-520, and 614-617 of SEQ ID NO:26; and at about amino acids 59-70, 124-140, and 257-262 of SEQ ID NO:29.

In a preferred embodiment, a 83378, 84233, 64708, 85041, or 84234 polypeptide or protein has at least one non-cytoplasmic loop or a region which includes at least about 4, preferably about 5-10, preferably about 10-20, and more preferably about 20-30 amino acid residues and has at least about 60%, 70%, 80%, 90%, 95%, 99%, or 100% homology with an “non-cytoplasmic loop,” e.g., at least one non-cytoplasmic loop of human 83378, 84233, 64708, 85041, or 84234 (e.g., residues 32-43, 99-114, and 266-282 of SEQ ID NO:17; residues 50-57, 114-127, and 192-200 of SEQ ID NO:20; residues 52-57, 120-136, and 220-231 of SEQ ID NO:23; residues 78-98, 146-151, 215-238, 289-303, 363-418, 506-520, and 614-617 of SEQ ID NO:26; and residues 59-70, 124-140, and 257-262 of SEQ ID NO:29).

In another embodiment, a 83378, 84233, 64708, 85041, or 84234 protein includes a “C-terminal cytoplasmic domain”, also referred to herein as a C-terminal cytoplasmic tail, in the sequence of the protein. As used herein, a “C-terminal cytoplasmic domain” includes an amino acid sequence having a length of at least about 30, preferably about 50-350, preferably about 60-250, more preferably about 80-220 amino acid residues and is located within a cell or within the cytoplasm of a cell. Accordingly, the N-terminal amino acid residue of a “C-terminal cytoplasmic domain” is adjacent to a C-terminal amino acid residue of a transmembrane domain in a 83378, 84233, 64708, 85041, or 84234 protein. For example, a C-terminal cytoplasmic domain is found at about amino acid residues 300-485 of SEQ ID NO:17; at about amino acid residues 219-320 of SEQ ID NO:20; at about amino acid residues 250-461 of SEQ ID NO:23; at about amino acid residues 642-765 of SEQ ID NO:26; and at about amino acid residues 287-376 of SEQ ID NO:29.

In a preferred embodiment, a 83378, 84233, 64708, 85041, or 84234 polypeptide or protein has a C-terminal cytoplasmic domain or a region which includes at least about 50, preferably about 150-550, more preferably about 50-70 amino acid residues and has at least about 60%, 70% 80% 90% 95%, 99%, or 100% homology with an “C-terminal cytoplasmic domain,” e.g., the C-terminal cytoplasmic domain of human 83378, 84233, 64708, 85041, or 84234 (e.g., residues 300-485 of SEQ ID NO:17; residues 219-320 of SEQ ID NO:20; residues 250-461 of SEQ ID NO:23; residues 642-765 of SEQ ID NO:26; and residues 287-376 of SEQ ID NO:29).

Histidine residues in metal transporter proteins play important roles in binding to divalent metal ions such as zinc. Histidine residues located in the cytoplasmic domain between the fourth and fifth transmembrane domains as well as those located in the C-terminal cytoplasmic domain of metal trasnporter proteins are thought to be of particular importance.

The 83378 protein has seven histidine residues in the C-terminal cytoplasmic domain (amino acid residues 300-485 of SEQ ID NO:17). A preferred 83378 polypeptide has at least one, preferably two, three, four, five, six, or seven histidine residues in a C-terminal cytoplasmic domain. An alignment of 83378 with rat ZnT-1 (SEQ ID NO:49) is shown in FIG. 20A. An alignment of 83378 with the amino acid sequence of human GenBank™ Accession Number AL359609 (SEQ ID NO:50) is shown in FIG. 20B.

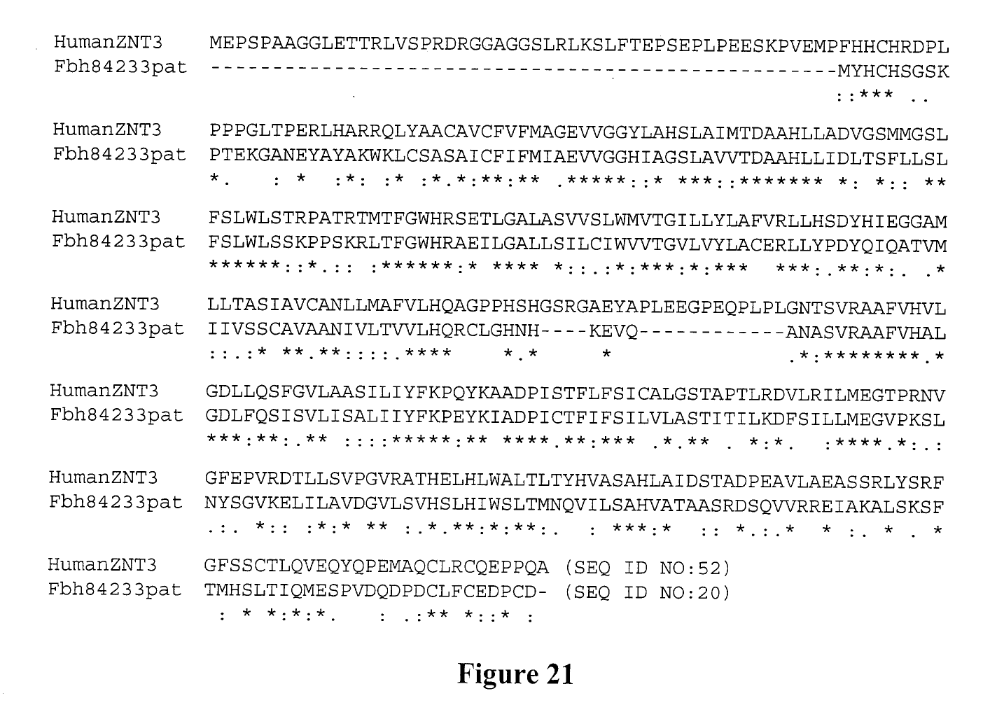

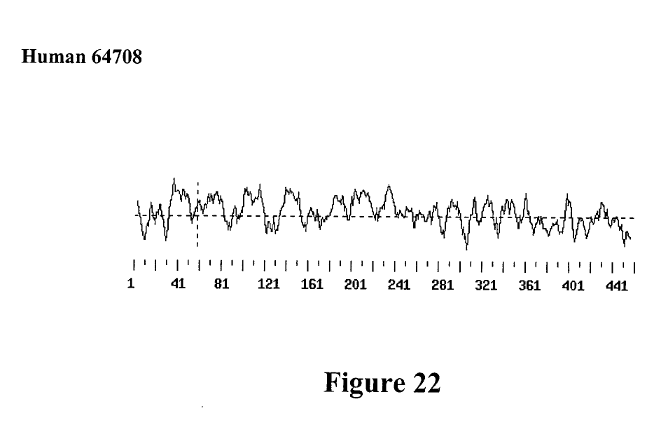

The 84233 protein has three histidine residues in the cytoplasmic domain between the fourth and fifth transmembrane domains (amino acid residues 148-166 of SEQ ID NO:20) and four histidine residues in the C-terminal cytoplasmic domain (amino acid residues 219-320 of SEQ ID NO:20). A preferred 84233 polypeptide has at least one, preferably two or three histidine residues in a cytoplasmic domain, e.g., a cytoplasmic domain located between the fourth and fifth transmembrane domains. A preferred 84233 polypeptide has at least one, preferably two, three, or four histidine residues in a C-terminal cytoplasmic domain. An alignment of 84233 with human ZnT-3 (SEQ ID NO:52) is shown in FIG. 21.

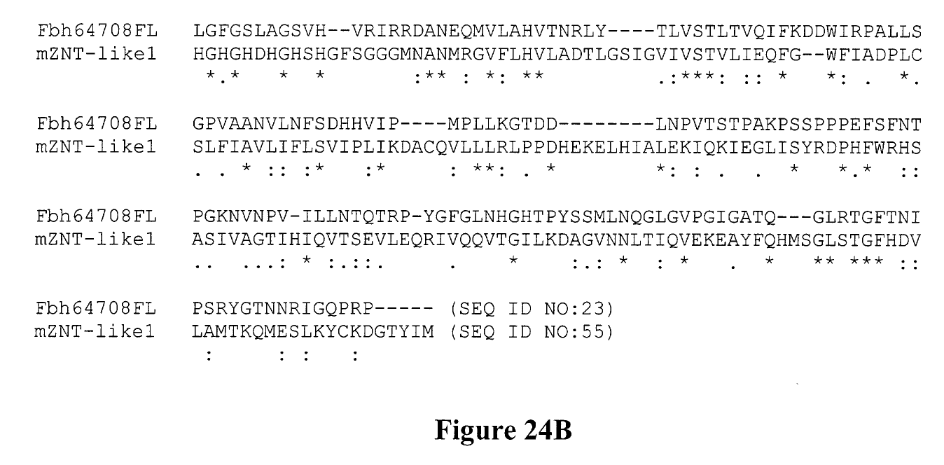

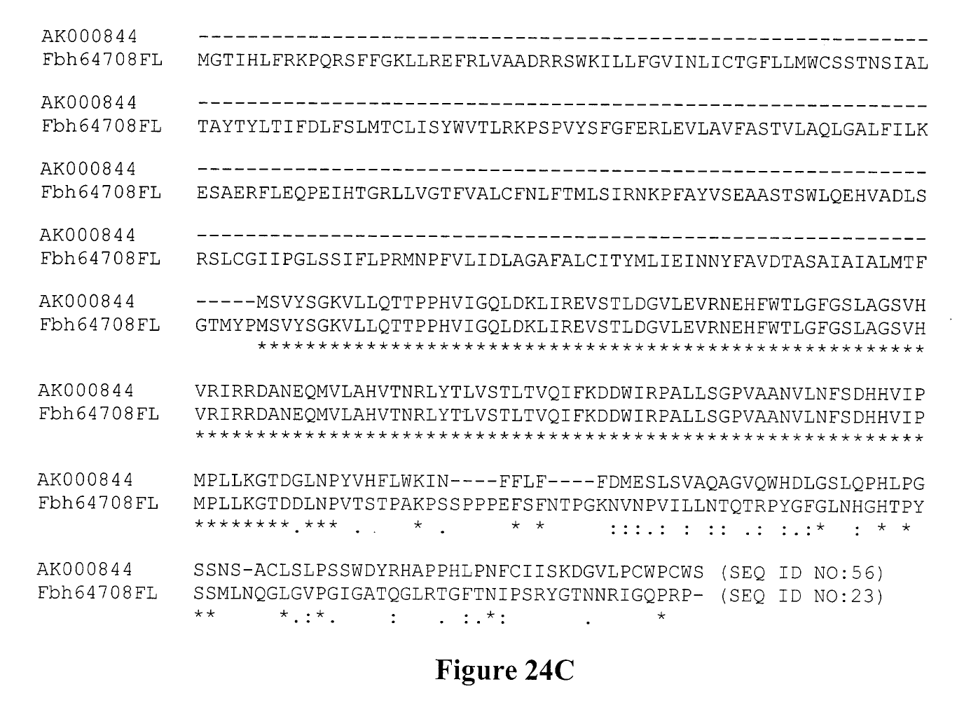

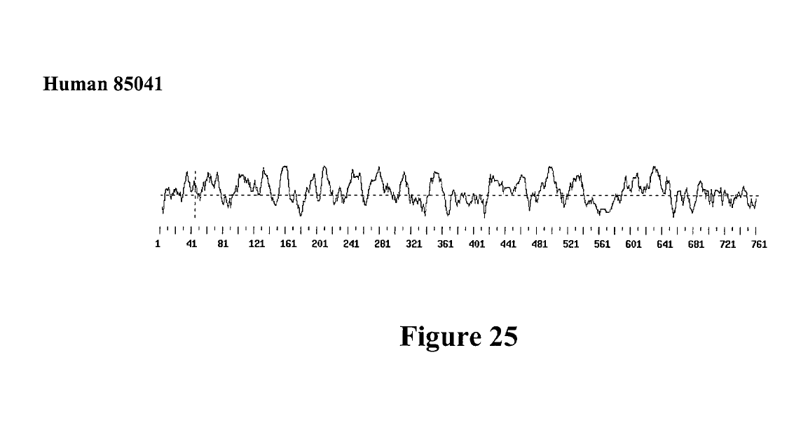

The 64708 protein has one histidine residue in the cytoplasmic domain between the fourth and fifth transmembrane domains (amino acid residues 156-201 of SEQ ID NO:23) and six histidine residues in the C-terminal cytoplasmic domain (amino acid residues 250-461 of SEQ ID NO:23). A preferred 64708 polypeptide has at least one histidine residue in a cytoplasmic domain, e.g., a cytoplasmic domain located between the fourth and fifth transmembrane domains. A preferred 64708 polypeptide has at least one, preferably two, three, four, five, or six histidine residues in a C-terminal cytoplasmic domain. An alignment of 64708 with murine ZnT-l1 (SEQ ID NO:55) is shown in FIG. 24A. An alignment of 64708 with the amino acid sequence of human GenBank™ Accession Number AK000844 (SEQ ID NO:56) is shown in FIG. 24B.

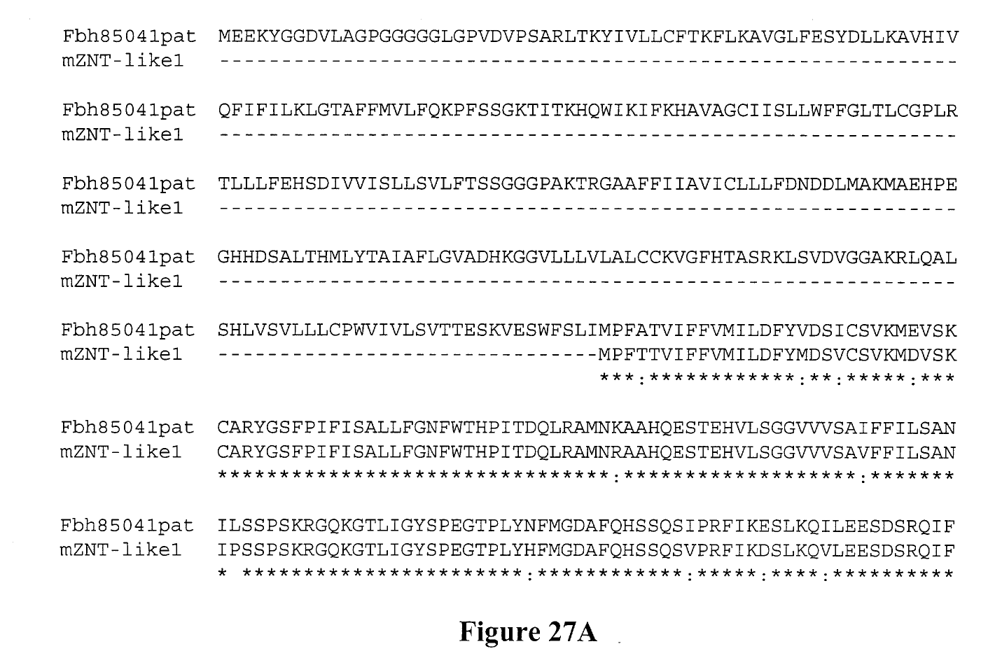

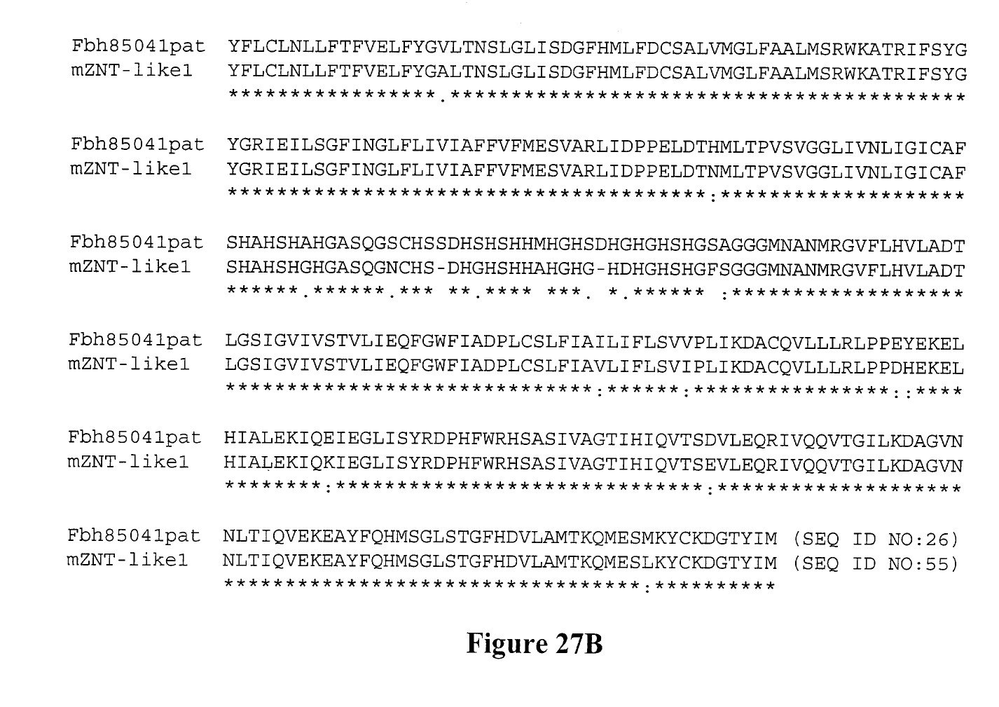

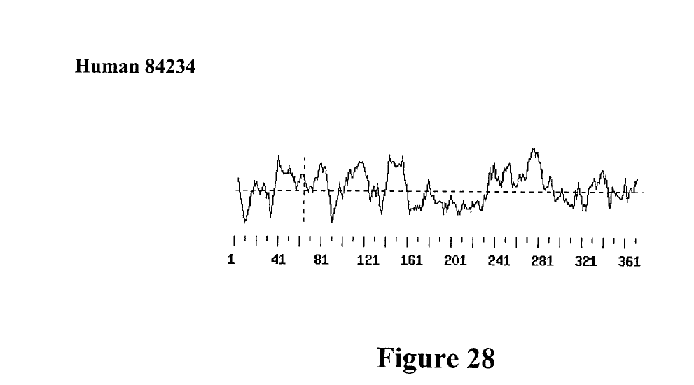

The 85041 protein has 15 histidine residue in the cytoplasmic domain between the twelfth and thirteenth transmembrane domains (amino acid residues 542-591 of SEQ ID NO:26) and six histidine residues in the C-terminal cytoplasmic domain (amino acid residues 642-765 of SEQ ID NO:26). A preferred 85041 polypeptide has at least one, preferably two, three, four, five, six, seven, eight, nine, 10, 11, 12, 13, 14, or 15 histidine residues in a cytoplasmic domain, e.g., a cytoplasmic domain located between the twelfth and thirteenth transmembrane domains. A preferred 85041 polypeptide has at least one, preferably two, three, four, five, or six histidine residues in a C-terminal cytoplasmic domain. An alignment of 85041 with murine ZnT-l1 (SEQ ID NO:55) is shown in FIG. 27.

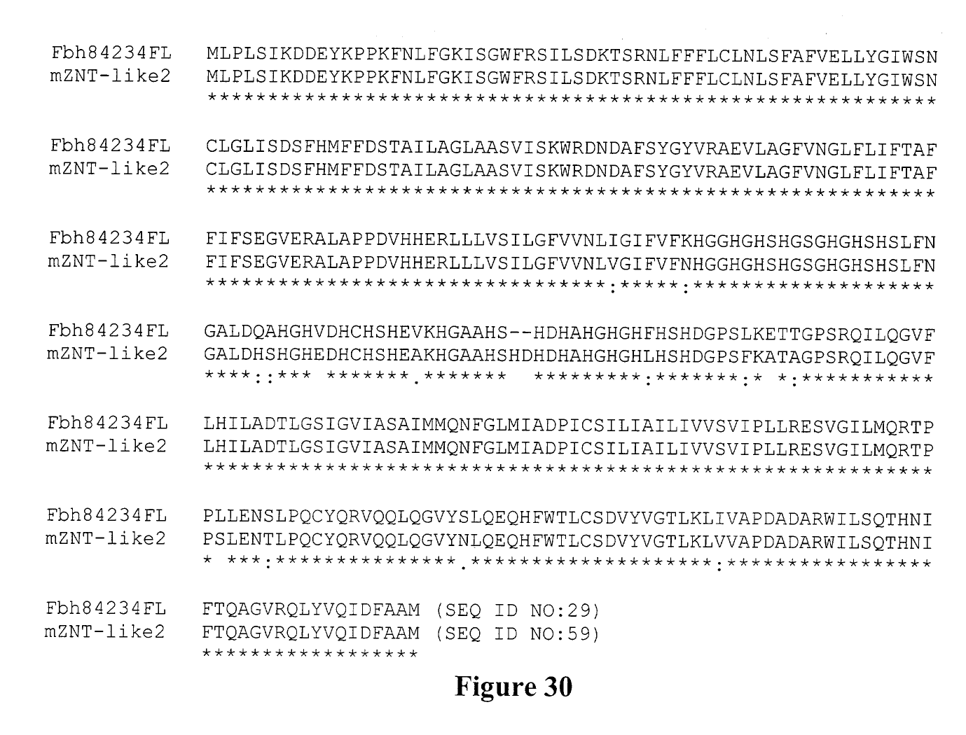

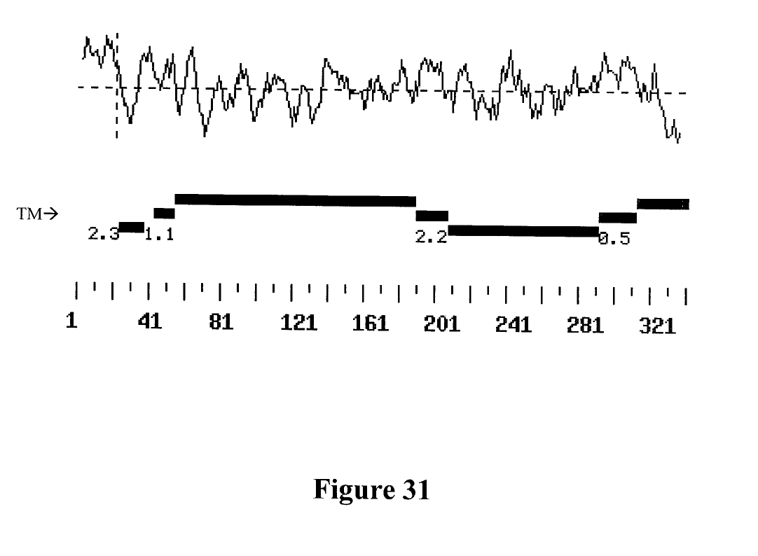

The 84234 protein has 21 histidine residue in the cytoplasmic domain between the fourth and fifth transmembrane domains (amino acid residues 160-236 of SEQ ID NO:29) and two histidine residues in the C-terminal cytoplasmic domain (amino acid residues 287-376 of SEQ ID NO:29). A preferred 84234 polypeptide has at least one, preferably two, three, four, five, six, seven, eight, nine, 10, 11, 12, 13, 14, 15, 16, 17, 18, 19, 20, or 21 histidine residues in a cytoplasmic domain, e.g., a cytoplasmic domainlocated between the fourth and fifth transmembrane domains. A preferred 84234 polypeptide has at least one, preferably two histidine residues in a C-terminal cytoplasmic domain. An alignrment of 84234 with murine ZnT-l2 (SEQ ID NO:59) is shown in FIG. 30.

A 83378, 84233, 64708, 85041, or 84234 family member can include a cation efflux domain and at least one, preferably two, three, four, five, or six transmembrane domains. A 85041 family member can further include seven, eight, nine, 10, 11, 12, 13, or 14 transmembrane domains. Furthermore, a 83378, 84233, 64708, 85041, or 84234 family member can include at least one, two, three, or preferably four cytoplasmic domains. A 85041 family member can further include five six, seven, or eight cytoplasmic domains. Furthermore, a 83378, 84233, 64708, 85041, or 84234 family member can include at least one, two, or preferably three non-cytoplasmic loops. A 85041 family member can further include four, five six, or seven, non-cytoplasmic loops.

As the 83378, 84233, 64708, 85041, or 84234 polypeptides of the invention may modulate 83378, 84233, 64708, 85041, or 84234-mediated activities, they may be useful as of for developing novel diagnostic and therapeutic agents for 83378, 84233, 64708, 85041, or 84234-mediated or related disorders, as described below.

As used herein, a “83378, 84233, 64708, 85041, or 84234 activity”, “biological activity of 83378, 84233, 64708, 85041, or 84234” or “functional activity of 83378, 84233, 64708, 85041, or 84234”, refers to an activity exerted by a 83378, 84233, 64708, 85041, or 84234 protein, polypeptide or nucleic acid molecule. For example, a 83378, 84233, 64708, 85041, or 84234 activity can be an activity exerted by 83378, 84233, 64708, 85041, or 84234 in a physiological milieu on, e.g., a 83378, 84233, 64708, 85041, or 84234-responsive cell or on a 83378, 84233, 64708, 85041, or 84234 substrate, e.g., a protein substrate. A 83378, 84233, 64708, 85041, or 84234 activity can be determined in vivo or in vitro. In one embodiment, a 83378, 84233, 64708, 85041, or 84234 activity is a direct activity, such as an association with a 83378, 84233, 64708, 85041, or 84234 target molecule. A “target molecule” or “binding partner” is a molecule with which a 83378, 84233, 64708, 85041, or 84234 protein binds or interacts in nature, e.g., a divalent metal ion, e.g., zinc. In an exemplary embodiment, 83378, 84233, 64708, 85041, or 84234 is a transporter of divalent metal ions.

A 83378, 84233, 64708, 85041, or 84234 activity can also be an indirect activity, e.g., a cellular signaling activity mediated by interaction of the 83378, 84233, 64708, 85041, or 84234 protein with a 83378, 84233, 64708, 85041, or 84234 receptor. The features of the 83378, 84233, 64708, 85041, or 84234 molecules of the present invention can provide similar biological activities as metal transporter family members. For example, the 83378, 84233, 64708, 85041, or 84234 proteins of the present invention can have one or more of the following activities: (1) modulate cellular tolerance and/or resistance to a metal ion, e.g., zinc; (2) facilitate cation diffusion; (3) modulate cellular efflux of a metal ion, e.g., zinc; (4) modulate vesicular sequestration of a metal ion, e.g., zinc; (5) modulate sequestration of a metal ion, e.g., zinc, in synaptic vesicles; or (6) bind to a metal ion, e.g., zinc.

In addition, the 83378, 84233, 64708, 85041, or 84234 molecules of the invention can be expected to function in tissues in which they are expressed. Thus, the 83378, 84233, 64708, 85041, or 84234 molecules can act as novel diagnostic targets and therapeutic agents for controlling metal transport-related disorders, e.g., disorders associated with cellular toxicity resulting from aberrant or deficient cation diffusion. Furthermore, 83378, 84233, 64708, 85041, or 84234 molecules can be used for metal detoxification, e.g., to treat cells or individuals containing excessive or unwanted amounts of metal ions.

Accordingly, the 47476, 67210, 49875, 46842, 33201, 83378, 84233, 64708, 85041, or 84234 molecules can act as novel diagnostic targets and therapeutic agents for controlling one or more of cellular proliferative and/or differentiative disorders, immunological disorders (e.g., inflammatory disorders), red blood cell disorders, viral diseases, neurological disorders (e.g., brain disorders), pain or metabolic disorders, liver disorders, kidney disorders, disorders of the small intestine, disorder of metal ion imbalance, protein trafficking disorders, cardiovascular disorders, and disorders associated with bone metabolism.

Examples of cellular proliferative and/or differentiative disorders include cancer, e.g., carcinoma, sarcoma, metastatic disorders or hematopoietic neoplastic disorders, e.g., leukemias. A metastatic tumor can arise from a multitude of primary tumor types, including but not limited to those of prostate, colon, lung, breast and liver origin.

As used herein, the terms “cancer”, “hyperproliferative” and “neoplastic” refer to cells having the capacity for autonomous growth. Examples of such cells include cells having an abnormal state or condition characterized by rapidly proliferating cell growth. Hyperproliferative and neoplastic disease states may be categorized as pathologic, i.e., characterizing or constituting a disease state, or may be categorized as non-pathologic, i.e., a deviation from normal but not associated with a disease state. The term is meant to include all types of cancerous growths or oncogenic processes, metastatic tissues or malignantly transformed cells, tissues, or organs, irrespective of histopathologic type or stage of invasiveness. “Pathologic hyperproliferative” cells occur in disease states characterized by malignant tumor growth. Examples of non-pathologic hyperproliferative cells include proliferation of cells associated with wound repair.

The terms “cancer” or “neoplasms” include malignancies of the various organ systems, such as affecting lung, breast, thyroid, lymphoid, gastrointestinal, and genito-urinary tract, as well as adenocarcinomas which include malignancies such as most colon cancers, renal-cell carcinoma, prostate cancer and/or testicular tumors, non-small cell carcinoma of the lung, cancer of the small intestine and cancer of the esophagus.

The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. Exemplary carcinomas include those forming from tissue of the cervix, lung, prostate, breast, head and neck, colon and ovary. The term also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures.

The term “sarcoma” is art recognized and refers to malignant tumors of mesenchymal derivation.

Examples of cellular proliferative and/or differentiative disorders of the colon include, but are not limited to, non-neoplastic polyps, adenomas, familial syndromes, colorectal carcinogenesis, colorectal carcinoma, and carcinoid tumors.

Examples of cellular proliferative and/or differentiative disorders of the liver include, but are not limited to, nodular hyperplasias, adenomas, and malignant tumors, including primary carcinoma of the liver and metastatic tumors.

Examples of cellular proliferative and/or differentiative disorders of the breast include, but are not limited to, proliferative breast disease including, e.g., epithelial hyperplasia, sclerosing adenosis, and small duct papillomas; tumors, e.g., stromal tumors such as fibroadenoma, phyllodes tumor, and sarcomas, and epithelial tumors such as large duct papilloma; carcinoma of the breast including in situ (noninvasive) carcinoma that includes ductal carcinoma in situ (including Paget's disease) and lobular carcinoma in situ, and invasive (infiltrating) carcinoma including, but not limited to, invasive ductal carcinoma, invasive lobular carcinoma, medullary carcinoma, colloid (mucinous) carcinoma, tubular carcinoma, and invasive papillary carcinoma, and miscellaneous malignant neoplasms. Disorders in the male breast include, but are not limited to, gynecomastia and carcinoma.

Examples of cellular proliferative and/or differentiative disorders of the lung include, but are not limited to, bronchogenic carcinoma, including paraneoplastic syndromes, bronchioloalveolar carcinoma, neuroendocrine tumors, such as bronchial carcinoid, miscellaneous tumors, and metastatic tumors; pathologies of the pleura, including inflammatory pleural effusions, noninflammatory pleural effusions, pneumothorax, and pleural tumors, including solitary fibrous tumors (pleural fibroma) and malignant mesothelioma.

Additional examples of proliferative disorders include hematopoietic neoplastic disorders. As used herein, the term “hematopoietic neoplastic disorders” includes diseases involving hyperplastic/neoplastic cells of hematopoietic origin. A hematopoietic neoplastic disorder can arise from myeloid, lymphoid or erythroid lineages, or precursor cells thereof. Preferably, the diseases arise from poorly differentiated acute leukemias, e.g., erythroblastic leukemia and acute megakaryoblastic leukemia. Additional exemplary myeloid disorders include, but are not limited to, acute promyeloid leukemia (APML), acute myelogenous leukemia (AML) and chronic myelogenous leukemia (CML) (reviewed in Vaickus, L. (1991) Crit Rev. in Oncol./Hemotol. 11:267-97); lymphoid malignancies include, but are not limited to acute lymphoblastic leukemia (ALL) which includes B-lineage ALL and T-lineage ALL, chronic lymphocytic leukemia (CLL), prolymphocytic leukemia (PLL), hairy cell leukemia (HLL) and Waldenstrom's macroglobulinemia (WM). Additional forms of malignant lymphomas include, but are not limited to non-Hodgkin lymphoma and variants thereof, peripheral T cell lymphomas, adult T cell leukemia/lymphoma (ATL), cutaneous T-cell lymphoma (CTCL), large granular lymphocytic leukemia (LGF), Hodgkin's disease and Reed-Stemberg disease.

Immunological disorders or “inflammatory disorders” include disorders that involve hematopoieitic cells, e.g., a myeloid, lymphoid or erythroid cell, or a precursor cell thereof. Examples of such cells include myelocytic cells (polymorphonuclear cells), erythrocytic cells, lymphocytes, monocytes, reticular cells, plasma cells and megakaryocytes, as well as stem cells for the different lineages, and precursors for the committed progenitor cells, for example, precursors of blood cells (e.g., red blood cells, such as erythroblasts), macrophages (monoblasts), platelets (megakaryocytes), polymorphonuclear leucocytes (myeloblasts), and lymphocytes (lymphoblasts). Immunological disorders or diseases include, but are not limited to, autoimmune diseases (including, for example, diabetes mellitus, arthritis (including rheumatoid arthritis, juvenile rheumatoid arthritis, osteoarthritis, psoriatic arthritis), multiple sclerosis, encephalomyelitis, myasthenia gravis, systemic lupus erythematosis, autoimmune thyroiditis, dermatitis (including atopic dermatitis and eczematous dermatitis), psoriasis, Sjogren's Syndrome, Crohn's disease, aphthous ulcer, iritis, conjunctivitis, keratoconjunctivitis, ulcerative colitis, asthma, allergic asthma, cutaneous lupus erythematosus, scleroderma, vaginitis, proctitis, drug eruptions, leprosy reversal reactions, erythema nodosum leprosum, autoimmune uveitis, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis, Wegener's granulomatosis, chronic active hepatitis, Stevens-Johnson syndrome, idiopathic sprue, lichen planus, Graves' disease, sarcoidosis, primary biliary cirrhosis, uveitis posterior, and interstitial lung fibrosis), graft-versus-host disease, cases of transplantation, and allergy such as, atopic allergy.

Disorders involving red blood cells include, but are not limited to, anemias, such as hemolytic anemias, including hereditary spherocytosis, hemolytic disease due to erythrocyte enzyme defects: glucose-6-phosphate dehydrogenase deficiency, sickle cell disease, thalassemia syndromes, paroxysmal nocturnal hemoglobinuria, immunohemolytic anemia, and hemolytic anemia resulting from trauma to red cells; and anemias of diminished erythropoiesis, including megaloblastic anemias, such as anemias of vitamin B12 deficiency: pernicious anemia, and anemia of folate deficiency, iron deficiency anemia, anemia of chronic disease, aplastic anemia, pure red cell aplasia, and other forms of marrow failure.

Disorders related to reduced platelet number, thrombocytopenia, include idiopathic thrombocytopenic purpura, including acute idiopathic thrombocytopenic purpura, drug-induced thrombocytopenia, HIV-associated thrombocytopenia, and thrombotic microangiopathies: thrombotic thrombocytopenic purpura and hemolytic-uremic syndrome.

Bone marrow disorders include but are not limited to the following: diseases involving hematopocitic stem cells; committed lymphoid progenitor cells; lymphoid cells including B and T-cells; committed myeloid progenitors, including monocytes, granulocytes, and megakaryocytes; and committed erythroid progenitors. These include but are not limited to the leukemias, including B-lymphoid leukemias, T-lymphoid leukemias, undifferentiated leukemias; erythroleukemia, megakaryoblastic leukemia, monocytic; [leukemias are encompassed with and without differentiation; chronic and acute lymphoblastic leukemia, chronic and acute lymphocytic leukemia, chronic and acute myelogenous leukemia, lymphoma, myelo dysplastic syndrome, chronic and acute myeloid leukemia, myelomonocytic leukemia; chronic and acute myeloblastic leukemia, chronic and acute myelogenous leukemia, chronic and acute promyclocytic leukemia, chronic and acute myelocytic leukemia, hematologic malignancies of monocyte-macrophage lineage, such as juvenile chronic myelogenous leukemia; secondary AML, antecedent hematological disorder; refractory anemia; aplastic anemia; reactive cutaneous angioendotheliomatosis; fibrosing disorders involving altered expression in dendritic cells, disorders including systemic sclerosis, E-M syndrome, epidemic toxic oil syndrome, eosinophilic fasciitis localized forms of scleroderrna, keloid, and fibrosing colonopathy; angiomatoid malignant fibrous histiocytoma; carcinoma, including primary head and neck squamous cell carcinoma; sarcoma, including kaposi's sarcoma; fibroadanoma and phyllodes tumors, including mammary fibroadenoma; stromal tumors; phyllodes tumors, including histiocytoma; erythroblastosis; neurofibromatosis; diseases of the vascular endothelium; demyelinating, particularly in old lesions; gliosis, vasogenic edema, vascular disease, Alzheimer's and Parkinson's disease; T-cell lymphomas; B-cell lymphomas.

Disorders involving the spleen include, but are not limited to, splenomegaly, including nonspecific acute splenitis, congestive spenomegaly, and spenic infarcts; neoplasms, congenital anomalies, and rupture. Disorders associated with splenomegaly include infections, such as nonspecific splenitis, infectious mononucleosis, tuberculosis, typhoid fever, brucellosis, cytomegalovirus, syphilis, malaria, histoplasmosis, toxoplasmosis, kala-azar, trypanosomiasis, schistosomiasis, leishmaniasis, and echinococcosis; congestive states related to partial hypertension, such as cirrhosis of the liver, portal or splenic vein thrombosis, and cardiac failure; lymphohematogenous disorders, such as Hodgkin disease, non-Hodgkin lymphomas/leukemia, multiple myeloma, myeloproliferative disorders, hemolytic anemias, and thrombocytopenic purpura; immunologic-inflammatory conditions, such as rheumatoid arthritis and systemic lupus erythematosus; storage diseases such as Gaucher disease, Niemann-Pick disease, and mucopolysaccharidoses; and other conditions, such as amyloidosis, primary neoplasms and cysts, and secondary neoplasms.

Examples of viral diseases include, but are not limited to, Hepatitis B, Hepatitis C and Herpes Simplex Virus (HSV). Modulators of 47476, 67210, and 46842 activity, in particular, could be used to control viral diseases. The modulators can be used in the treatment and/or diagnosis of viral infected tissue or virus-associated tissue fibrosis, especially liver and liver fibrosis. Also, 47476, 67210, and 46842 modulators can be used in the treatment and/or diagnosis of virus-associated carcinoma, especially hepatocellular cancer.

Neurological disorders or disorders involving the brain include, but are not limited to, disorders involving neurons, and disorders involving glia, such as astrocytes, oligodendrocytes, ependymal cells, and microglia; cerebral edema, raised intracranial pressure and herniation, and hydrocephalus; malformations and developmental diseases, such as neural tube defects, forebrain anomalies, posterior fossa anomalies, and syringomyelia and hydromyelia; perinatal brain injury; cerebrovascular diseases, such as those related to hypoxia, ischemia, and infarction, including hypotension, hypoperfusion, and low-flow states—global cerebral ischemia and focal cerebral ischemia—infarction from obstruction of local blood supply, intracranial hemorrhage, including intracerebral (intraparenchymal) hemorrhage, subarachnoid hemorrhage and ruptured berry aneurysms, and vascular malformations, hypertensive cerebrovascular disease, including lacunar infarcts, slit hemorrhages, and hypertensive encephalopathy; infections, such as acute meningitis, including acute pyogenic (bacterial) meningitis and acute aseptic (viral) meningitis, acute focal suppurative infections, including brain abscess, subdural empyema, and extradural abscess, chronic bacterial meningoencephalitis, including tuberculosis and mycobacterioses, neurosyphilis, and neuroborreliosis (Lyme disease), viral meningoencephalitis, including arthropod-borne (Arbo) viral encephalitis, Herpes simplex virus Type 1, Herpes simplex virus Type 2, Varicalla-zoster virus (Herpes zoster), cytomegalovirus, poliomyelitis, rabies, and human immunodeficiency virus 1, including HIV-1 meningoencephalitis (subacute encephalitis), vacuolar myelopathy, AIDS-associated myopathy, peripheral neuropathy, and AIDS in children, progressive multifocal leukoencephalopathy, subacute sclerosing panencephalitis, fungal meningoencephalitis, other infectious diseases of the nervous system; transmissible spongiform encephalopathies (prion diseases); demyelinating diseases, including multiple sclerosis, multiple sclerosis variants, acute disseminated encephalomyelitis and acute necrotizing hemorrhagic encephalomyelitis, and other diseases with demyelination; degenerative diseases, such as degenerative diseases affecting the cerebral cortex, including Alzheimer disease and Pick disease, degenerative diseases of basal ganglia and brain stem, including Parkinsonism, idiopathic Parkinson disease (paralysis agitans), progressive supranuclear palsy, corticobasal degenration, multiple system atrophy, including striatonigral degenration, Shy-Drager syndrome, and olivopontocerebellar atrophy, and Huntington disease; spinocerebellar degenerations, including spinocerebellar ataxias, including Friedreich ataxia, and ataxia-telanglectasia, degenerative diseases affecting motor neurons, including amyotrophic lateral sclerosis (motor neuron disease), bulbospinal atrophy (Kennedy syndrome), and spinal muscular atrophy; inborn errors of metabolism, such as leukodystrophies, including Krabbe disease, metachromatic leukodystrophy, adrenoleukodystrophy, Pelizaeus-Merzbacher disease, and Canavan disease, mitochondrial encephalomyopathies, including Leigh disease and other mitochondrial encephalomyopathies; toxic and acquired metabolic diseases, including vitamin deficiencies such as thiamine (vitamin B1) deficiency and vitamin B12 deficiency, neurologic sequelae of metabolic disturbances, including hypoglycemia, hyperglycemia, and hepatic encephatopathy, toxic disorders, including carbon monoxide, methanol, ethanol, and radiation, including combined methotrexate and radiation-induced injury; tumors, such as gliomas, including astrocytoma, including fibrillary (diffuse) astrocytoma and glioblastoma multiforme, pilocytic astrocytoma, pleomorphic xanthoastrocytoma, and brain stem glioma, oligodendroglioma, and ependymoma and related paraventricular mass lesions, neuronal tumors, poorly differentiated neoplasms, including medulloblastoma, other parenchymal tumors, including primary brain lymphoma, germ cell tumors, and pineal parenchymal tumors, meningiomas, metastatic tumors, paraneoplastic syndromes, peripheral nerve sheath tumors, including schwannoma, neurofibroma, and malignant peripheral nerve sheath tumor (malignant schwannoma), and neurocutaneous syndromes (phakomatoses), including neurofibromotosis, including Type 1 neurofibromatosis (NF1) and TYPE 2 neurofibromatosis (NF2), tuberous sclerosis, and Von Hippel-Lindau disease.

Diseases of metabolic imbalance include, but are not limited to, obesity, anorexia nervosa, cachexia, lipid disorders, and diabetes. Examples of pain disorders include, but are not limited to, pain response elicited during various forms of tissue injury, e.g., inflammation, infection, and ischemia, usually referred to as hyperalgesia (described in, for example, Fields, H. L. (1987) Pain, New York:McGraw-Hill); pain associated with musculoskeletal disorders, e.g., joint pain; tooth pain; headaches; pain associated with surgery; pain related to irritable bowel syndrome; or chest pain.

Liver disorders can be associated with an accumulation in the liver of fibrous tissue, such as that resulting from an imbalance between production and degradation of the extracellular matrix accompanied by the collapse and condensation of preexisting fibers. In addition, liver disorders can involve hepatocellular necrosis or injury induced by a wide variety of agents including processes which disturb homeostasis, such as an inflammatory process, tissue damage resulting from toxic injury or altered hepatic blood flow, and infections (e.g., bacterial, viral and parasitic). Causes of liver fibrosis include, but are not limited to portal hypertension, inborn errors of metabolism, for example, fibrosis resulting from a storage disorder such as Gaucher's disease (lipid abnormalities) or a glycogen storage disease, A1-antitrypsin deficiency, a disorder mediating the accumulation (e.g., storage) of an exogenous substance, for example, hemochromatosis (iron-overload syndrome) and copper storage diseases (Wilson's disease), disorders resulting in the accumulation of a toxic metabolite (e.g., tyrosinemia, fructosemia and galactosemia) and peroxisomal disorders (e.g., Zellweger syndrome), or the administration of various chemicals or drugs, such as methotrexate, isonizaid, oxyphenisatin, methyldopa, chlorpromazine, tolbutamide or alcohol, or a vascular disorder such as obstruction of either the intrahepatic or extrahepatic bile flow or an alteration in hepatic circulation resulting, for example, from chronic heart failure, veno-occlusive disease, portal vein thrombosis or Budd-Chiari syndrome.

Disorders involving the kidney include, but are not limited to, congenital anomalies including, but not limited to, cystic diseases of the kidney, that include but are not limited to, cystic renal dysplasia, autosomal dominant (adult) polycystic kidney disease, autosomal recessive (childhood) polycystic kidney disease, and cystic diseases of renal medulla, which include, but are not limited to, medullary sponge kidney, and nephronophthisis-uremic medullary cystic disease complex, acquired (dialysis-associated) cystic disease, such as simple cysts; glomerular diseases including pathologies of glomerular injury that include, but are not limited to, in situ immune complex deposition, that includes, but is not limited to, anti-GBM nephritis, Heymann nephritis, and antibodies against planted antigens, circulating immune complex nephritis, antibodies to glomerular cells, cell-mediated immunity in glomerulonephritis, activation of alternative complement pathway, epithelial cell injury, and pathologies involving mediators of glomerular injury including cellular and soluble mediators, acute glomerulonephritis, such as acute proliferative (poststreptococcal, postinfectious) glomerulonephritis, including but not limited to, poststreptococcal glomerulonephritis and nonstreptococcal acute glomerulonephritis, rapidly progressive (crescentic) glomerulonephritis, nephrotic syndrome, membranous glomerulonephritis (membranous nephropathy), minimal change disease (lipoid nephrosis), focal segmental glomerulosclerosis, membranoproliferative glomerulonephritis, IgA nephropathy (Berger disease), focal proliferative and necrotizing glomerulonephritis (focal glomerulonephritis), hereditary nephritis, including but not limited to, Alport syndrome and thin membrane disease (benign familial hematuria), chronic glomerulonephritis, glomerular lesions associated with systemic disease, including but not limited to, systemic lupus erythematosus, Henoch-Schonlein purpura, bacterial endocarditis, diabetic glomerulosclerosis, amyloidosis, fibrillary and immunotactoid glomerulonephritis, and other systemic disorders; diseases affecting tubules and interstitium, including acute tubular necrosis and tubulointerstitial nephritis, including but not limited to, pyelonephritis and urinary tract infection, acute pyelonephritis, chronic pyelonephritis and reflux nephropathy, and tubulointerstitial nephritis induced by drugs and toxins, including but not limited to, acute drug-induced interstitial nephritis, analgesic abuse nephropathy, nephropathy associated with nonsteroidal anti-inflammatory drugs, and other tubulointerstitial diseases including, but not limited to, urate nephropathy, hypercalcemia and nephrocalcinosis, and multiple myeloma; diseases of blood vessels including benign nephrosclerosis, malignant hypertension and accelerated nephrosclerosis, renal artery stenosis, and thrombotic microangiopathies including, but not limited to, classic (childhood) hemolytic-uremic syndrome, adult hemolytic-uremic syndrome/thrombotic thrombocytopenic purpura, idiopathic HUS/TTP, and other vascular disorders including, but not limited to, atherosclerotic ischemic renal disease, atheroembolic renal disease, sickle cell disease nephropathy, diffuse cortical necrosis, and renal infarcts; urinary tract obstruction (obstructive uropathy); urolithiasis (renal calculi, stones); and tumors of the kidney including, but not limited to, benign tumors, such as renal papillary adenoma, renal fibroma or hamartoma (renomedullary interstitial cell tumor), angiomyolipoma, and oncocytoma, and malignant tumors, including renal cell carcinoma (hypemephroma, adenocarcinoma of kidney), which includes urothelial carcinomas of renal pelvis.

Disorders involving the small intestine include the malabsorption syndromes such as, celiac sprue, tropical sprue (postinfectious sprue), whipple disease, disaccharidase (lactase) deficiency, abetalipoproteinemia, and tumors of the small intestine including adenomas and adenocarcinoma.

As used herein, disorders involving the heart, or “cardiovascular disease” or a “cardiovascular disorder” include diseases or disorders that affect the cardiovascular system, e.g., the heart, the blood vessels, and/or the blood. A cardiovascular disorder can be caused by an imbalance in arterial pressure, a malfunction of the heart, or an occlusion of a blood vessel, e.g., by a thrombus. A cardiovascular disorder includes, but is not limited to disorders such as arteriosclerosis, atherosclerosis, cardiac hypertrophy, ischemia reperfusion injury, restenosis, arterial inflammation, vascular wall remodeling, ventricular remodeling, rapid ventricular pacing, coronary microembolism, tachycardia, bradycardia, pressure overload, aortic bending, coronary artery ligation, vascular heart disease, valvular disease, including but not limited to, valvular degeneration caused by calcification, rheumatic heart disease, endocarditis, or complications of artificial valves; atrial fibrillation, long-QT syndrome, congestive heart failure, sinus node dysfunction, angina, heart failure, hypertension, atrial fibrillation, atrial flutter, pericardial disease, including but not limited to, pericardial effusion and pericarditis; cardiomyopathies, e.g., dilated cardiomyopathy or idiopathic cardiomyopathy, myocardial infarction, coronary artery disease, coronary artery spasm, ischemic disease, arrhythmia, sudden cardiac death, and cardiovascular developmental disorders (e.g., arteriovenous malformations, arteriovenous fistulae, raynaud's syndrome, neurogenic thoracic outlet syndrome, causalgia/reflex sympathetic dystrophy, hemangioma, aneurysm, cavernous angioma, aortic valve stenosis, atrial septal defects, atrioventricular canal, coarctation of the aorta, ebsteins anomaly, hypoplastic left heart syndrome, interruption of the aortic arch, mitral valve prolapse, ductus arteriosus, patent foramen ovale, partial anomalous pulmonary venous return, pulmonary atresia with ventricular septal defect, pulmonary atresia without ventricular septal defect, persistance of the fetal circulation, pulmonary valve stenosis, single ventricle, total anomalous pulmonary venous return, transposition of the great vessels, tricuspid atresia, truncus arteriosus, ventricular septal defects). A cardiovasular disease or disorder also can include an endothelial cell disorder and a hematological disorder.

A hematological disorder can include thrombosis. Thrombosis can result from platelet dysfunction, e.g., seen in myocardial infarction, angina, hypertension, lipid disorders, diabetes mellitus; myelodysplastic syndromes; mycloproliferative syndromes (including polycythemia vera and thombocythemia); thrombotic thrombocytopenic purpuras; HIV-induced platelet disorders (AIDS-Thrombocytopenia); heparin induced thrombocytopenia; mural cell alterations/interactions leading to platelet aggregation/degranulation, vascular endothelial cell activation/injury, monocyte/macrophage extravasation and smooth muscle cell proliferation; autoimmune disorders such as, but not limited to vasculitis, antiphospholipid syndromes, systemic lupus erythromatosis; inflammatory diseases, such as, but not limited to immune activation; graft vs. host disease; radiation induced hypercoagulation; clotting factor dysregulation either hereditary (autosomal dominant or recessive) such as, but not limited to clotting factor pathways including protein C/S, Anti-thrombin III deficiency, and the Factor V Leiden mutation or acquired such as but not limited to autoimmune, cancer-associated and drug-induced dysregulation of clotting factors.

As used herein, an “endothelial cell disorder” includes a disorder characterized by aberrant, unregulated, or unwanted endothelial cell activity, e.g., proliferation, migration, angiogenesis, or vascularization; or aberrant expression of cell surface adhesion molecules or genes associated with angiogenesis, e.g., TIE-2, FLT and FLK. Endothelial cell disorders include tumorigenesis, tumor metastasis, psoriasis, diabetic retinopathy, endometriosis, Grave's disease, ischemic disease (e.g., atherosclerosis), and chronic inflammatory diseases (e.g., rheumatoid arthritis).

Disorders involving blood vessels include, but are not limited to, responses of vascular cell walls to injury, such as endothelial dysfunction and endothelial activation and intimal thickening; vascular diseases including, but not limited to, congenital anomalies, such as arteriovenous fistula, atherosclerosis, and hypertensive vascular disease, such as hypertension; inflammatory disease—the vasculitides, such as giant cell (temporal) arteritis, Takayasu arteritis, polyarteritis nodosa (classic), Kawasaki syndrome (mucocutaneous lymph node syndrome), microscopic polyanglitis (microscopic polyarteritis, hypersensitivity or leukocytoclastic anglitis), Wegener granulomatosis, thromboanglitis obliterans (Buerger disease), vasculitis associated with other disorders, and infectious arteritis; Raynaud disease; aneurysms and dissection, such as abdominal aortic aneurysms, syphilitic (luetic) aneurysms, and aortic dissection (dissecting hematoma); disorders of veins and lymphatics, such as varicose veins, thrombophlebitis and phlebothrombosis, obstruction of superior vena cava (superior vena cava syndrome), obstruction of inferior vena cava (inferior vena cava syndrome), and lymphangitis and lymphedema; tumors, including benign tumors and tumor-like conditions, such as hemangioma, lymphangioma, glomus tumor (glomangioma), vascular ectasias, and bacillary angiomatosis, and intermediate-grade (borderline low-grade malignant) tumors, such as Kaposi sarcoma and hemangloendothelioma, and malignant tumors, such as angiosarcoma and hemangiopericytoma; and pathology of therapeutic interventions in vascular disease, such as balloon angioplasty and related techniques and vascular replacement, such as coronary artery bypass graft surgery.

“Bone metabolism” refers to direct or indirect effects in the formation or degeneration of bone structures, e.g., bone formation, bone resorption, etc., which may ultimately affect the concentrations in serum of calcium and phosphate. This term also includes activities mediated by proteins or small molecules that influence bone cells, e.g. osteoclasts and osteoblasts, resulting in bone formation and degeneration, such as the stimulation of differentiation of monocytes and mononuclear phagocytes into osteoclasts. Examples of bone metabolism disorders include, but are not limited to, osteoporosis, osteodystrophy, osteomalacia, rickets, osteitis fibrosa cystica, renal osteodystrophy, osteosclerosis, anti-convulsant treatment, osteopenia, fibrogenesis-imperfecta ossium, secondary hyperparathyrodism, hypoparathyroidism, hyperparathyroidism, cirrhosis, obstructive jaundice, drug induced metabolism, medullary carcinoma, chronic renal disease, rickets, sarcoidosis, glucocorticoid antagonism, malabsorption syndrome, steatorrhea, tropical sprue, idiopathic hypercalcemia and milk fever.

The 47476, 67210, 49875, 46842, 33201, 83378, 84233, 64708, 85041, and 84234 proteins, fragments thereof, and derivatives and other variants of the sequences in SEQ ID NO:2, SEQ ID NO:5, SEQ ID NO:8, SEQ ID NO:11, SEQ ID NO:14, SEQ ID NO:17, SEQ ID NO:20, SEQ ID NO:23, SEQ ID NO:26, and SEQ ID NO:29 thereof are collectively referred to as “polypeptides or proteins of the invention” or “47476”, “67210”, “49875”, “46842”, “33201”, “83378”, “84233”, “64708”, “85041”, or “84234” polypeptides or proteins. Nucleic acid molecules encoding such polypeptides or proteins are collectively referred to as “nucleic acids of the invention” or “47476”, “67210”, “49875”, “46842”, “33201”, “83378”, “84233”, “64708”, “85041”, and “84234” nucleic acids. 47476, 67210, 49875, 46842, 33201, 83378, 84233, 64708, 85041, and 84234 molecules refer to 47476, 67210, 49875, 46842, 33201, 83378, 84233, 64708, 85041, or 84234 nucleic acids, polypeptides, and antibodies.

As used herein, the term “nucleic acid molecule” includes DNA molecules (e.g., a cDNA or genomic DNA), RNA molecules (e.g., an mRNA) and analogs of the DNA or RNA. A DNA or RNA analog can be synthesized from nucleotide analogs. The nucleic acid molecule can be single-stranded or double-stranded, but preferably is double-stranded DNA.

The term “isolated nucleic acid molecule” or “purified nucleic acid molecule” includes nucleic acid molecules that are separated from other nucleic acid molecules present in the natural source of the nucleic acid. For example, with regards to genomic DNA, the term “isolated” includes nucleic acid molecules which are separated from the chromosome with which the genomic DNA is naturally associated. Preferably, an “isolated” nucleic acid is free of sequences which naturally flank the nucleic acid (i.e., sequences located at the 5′ and/or 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb or 0.1 kb of 5′ and/or 3′ nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material, or culture medium when produced by recombinant techniques, or substantially free of chemical precursors or other chemicals when chemically synthesized.

As used herein, the term “hybridizes under low stringency, medium stringency, high stringency, or very high stringency conditions” describes conditions for hybridization and washing. Guidance for performing hybridization reactions can be found in Current Protocols in Molecular Biology, John Wiley & Sons, N.Y. (1989), 6.3.1-6.3.6, which is incorporated by reference. Aqueous and nonaqueous methods are described in that reference and either can be used. Specific hybridization conditions referred to herein are as follows: 1) low stringency hybridization conditions in 6× sodium chloride/sodium citrate (SSC) at about 45° C., followed by two washes in 0.2× SSC, 0.1% SDS at least at 50° C. (the temperature of the washes can be increased to 55° C. for low stringency conditions); 2) medium stringency hybridization conditions in 6× SSC at about 45° C., followed by one or more washes in 0.2× SSC, 0.1% SDS at 60° C.; 3) high stringency hybridization conditions in 6× SSC at about 45° C., followed by one or more washes in 0.2× SSC, 0.1% SDS at 65° C.; and preferably 4) very high stringency hybridization conditions are 0.5M sodium phosphate, 7% SDS at 65° C., followed by one or more washes at 0.2× SSC, 1% SDS at 65° C. Very high stringency conditions (4) are the preferred conditions and the ones that should be used unless otherwise specified.

Preferably, an isolated nucleic acid molecule of the invention that hybridizes under a stringency condition described herein to the sequence of SEQ ID NO:1, SEQ ID NO:4, SEQ ID NO:7, SEQ ID NO:10, SEQ ID NO:13, SEQ ID NO:16, SEQ ID NO:19, SEQ ID NO:22, SEQ ID NO:25, or SEQ ID NO:28, corresponds to a naturally-occurring nucleic acid molecule.

As used herein, a “naturally-occurring” nucleic acid molecule refers to an RNA or DNA molecule having a nucleotide sequence that occurs in nature. For example a naturally occurring nucleic acid molecule can encode a natural protein.

As used herein, the terms “gene” and “recombinant gene” refer to nucleic acid molecules which include at least an open reading frame encoding a 47476, 67210, 49875, 46842, 33201, 83378, 84233, 64708, 85041, or 84234 protein. The gene can optionally further include non-coding sequences, e.g., regulatory sequences and introns. Preferably, a gene encodes a mammalian 47476, 67210, 49875, 46842, 33201, 83378, 84233, 64708, 85041, or 84234 protein or derivative thereof.

An “isolated” or “purified” polypeptide or protein is substantially free of cellular material or other contaminating proteins from the cell or tissue source from which the protein is derived, or substantially free from chemical precursors or other chemicals when chemically synthesized. “Substantially free” means that a preparation of 47476, 67210, 49875, 46842, 33201, 83378, 84233, 64708, 85041, or 84234 protein is at least 10% pure. In a preferred embodiment, the preparation of 47476, 67210, 49875, 46842, 33201, 83378, 84233, 64708, 85041, or 84234 protein has less than about 30%, 20%, 10% and more preferably 5% (by dry weight), of non-47476, 67210, 49875, 46842, 33201, 83378, 84233, 64708, 85041, or 84234 protein (also referred to herein as a “contaminating protein”), or of chemical precursors or non-47476, 67210, 49875, 46842, 33201, 83378, 84233, 64708, 85041, or 84234 chemicals. When the 47476, 67210, 49875, 46842, 33201, 83378, 84233, 64708, 85041, or 84234 protein or biologically active portion thereof is recombinantly produced, it is also preferably substantially free of culture medium, i.e., culture medium represents less than about 20%, more preferably less than about 10%, and most preferably less than about 5% of the volume of the protein preparation. The invention includes isolated or purified preparations of at least 0.01, 0.1, 1.0, and 10 milligrams in dry weight.

A “non-essential” amino acid residue is a residue that can be altered from the wild-type sequence of 47476, 67210, 49875, 46842, 33201, 83378, 84233, 64708, 85041, or 84234 without abolishing or substantially altering a 47476, 67210, 49875, 46842, 33201, 83378, 84233, 64708, 85041, or 84234 activity. Preferably the alteration does not substantially alter the 47476, 67210, 49875, 46842, 33201, 83378, 84233, 64708, 85041, or 84234 activity, e.g., the activity is at least 20%, 40%, 60%, 70% or 80% of wild-type. An “essential” amino acid residue is a residue that, when altered from the wild-type sequence of 47476, 67210, 49875, 46842, 33201, 83378, 84233, 64708, 85041, or 84234, results in abolishing a 47476, 67210, 49875, 46842, 33201, 83378, 84233, 64708, 85041, or 84234 activity such that less than 20% of the wild-type activity is present. For example, conserved amino acid residues in 47476, 67210, 49875, 46842, 33201, 83378, 84233, 64708, 85041, or 84234 are predicted to be particularly unamenable to alteration.

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in a 47476, 67210, 49875, 46842, 33201, 83378, 84233, 64708, 85041, or 84234 protein is preferably replaced with another amino acid residue from the same side chain family. Alternatively, in another embodiment, mutations can be introduced randomly along all or part of a 47476, 67210, 49875, 46842, 33201, 83378, 84233, 64708, 85041, or 84234 coding sequence, such as by saturation mutagenesis, and the resultant mutants can be screened for 47476, 67210, 49875, 46842, 33201, 83378, 84233, 64708, 85041, or 84234 biological activity to identify mutants that retain activity. Following mutagenesis of SEQ ID NO:1, SEQ ID NO:4, SEQ ID NO:7, SEQ ID NO:10, SEQ ID NO:13, SEQ ID NO:16, SEQ ID NO:19, SEQ ID NO:22, SEQ ID NO:25, SEQ ID NO:28, SEQ ID NO:3, SEQ ID NO:6, SEQ ID NO:9, SEQ ID NO:12, SEQ ID NO:15, SEQ ID NO:18, SEQ ID NO:21, SEQ ID NO:24, SEQ ID NO:27, or SEQ ID NO:30, the encoded protein can be expressed recombinantly and the activity of the protein can be determined.

As used herein, a “biologically active portion” of a 47476, 67210, 49875, 46842, 33201, 83378, 84233, 64708, 85041, or 84234 protein includes a fragment of a 47476, 67210, 49875, 46842, 33201, 83378, 84233, 64708, 85041, or 84234 protein which participates in an interaction, e.g., an intramolecular or an inter-molecular interaction. An inter-molecular interaction can be a specific binding interaction or an enzymatic interaction (e.g., the interaction can be transient and a covalent bond is formed or broken). An inter-molecular interaction can be between a 47476, 67210, 49875, 46842, 33201, 83378, 84233, 64708, 85041, or 84234 molecule and a non-47476, 67210, 49875, 46842, 33201, 83378, 84233, 64708, 85041, or 84234 molecule or between a first 47476, 67210, 49875, 46842, 33201, 83378, 84233, 64708, 85041, or 84234 molecule and a second 47476, 67210, 49875, 46842, 33201, 83378, 84233, 64708, 85041, or 84234 molecule (e.g., a dimerization interaction). Biologically active portions of a 47476, 67210, 49875, 46842, 33201, 83378, 84233, 64708, 85041, or 84234 protein include peptides comprising amino acid sequences sufficiently homologous to or derived from the amino acid sequence of the 47476, 67210, 49875, 46842, 33201, 83378, 84233, 64708, 85041, or 84234 protein, e.g., the amino acid sequence shown in SEQ ID NO:2, SEQ ID NO:5, SEQ ID NO:8, SEQ ID NO:11, SEQ ID NO:14, SEQ ID NO:17, SEQ ID NO:20, SEQ ID NO:23, SEQ ID NO:26, or SEQ ID NO:29, which include less amino acids than the full length 47476, 67210, 49875, 46842, 33201, 83378, 84233, 64708, 85041, or 84234 proteins, and exhibit at least one activity of a 47476, 67210, 49875, 46842, 33201, 83378, 84233, 64708, 85041, or 84234 protein. Typically, biologically active portions comprise a domain or motif with at least one activity of the 47476, 67210, 49875, 46842, 33201, 83378, 84233, 64708, 85041, or 84234 protein, e.g., stimulation of guanine nucleoside dissociation from a GTPase protein, transfer of a sugar residue to another molecule, unwinding of a duplex nucleic acid molecule, stimulation of the hydrolysis of GTP molecule bound to a GTPase protein, catalyzsis of the oxidation of an alcohol group present on a molecule, or transport of metal ions across a lipid bilayer. A biologically active portion of a 47476, 67210, 49875, 46842, 33201, 83378, 84233, 64708, 85041, or 84234 protein can be a polypeptide which is, for example, 10, 25, 50, 100, 200 or more amino acids in length. Biologically active portions of a 47476, 67210, 49875, 46842, 33201, 83378, 84233, 64708, 85041, or 84234 protein can be used as targets for developing agents which modulate a 47476, 67210, 49875, 46842, 33201, 83378, 84233, 64708, 85041, or 84234 mediated activity, e.g., stimulation of guanine nucleoside dissociation from a GTPase protein, transfer of a sugar residue to another molecule, unwinding of a duplex nucleic acid molecule, stimulation of the hydrolysis of GTP molecule bound to a GTPase protein, catalyzsis of the oxidation of an alcohol group present on a molecule, or transport of metal ions across a lipid bilayer.

Calculations of homology or sequence identity between sequences (the terms are used interchangeably herein) are performed as follows.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, 60%, and even more preferably at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”).

The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch ((1970) J. Mol. Biol. 48:444-453) algorithm which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. A particularly preferred set of parameters (and the one that should be used unless otherwise specified) are a Blossum 62 scoring matrix with a gap penalty of 12, a gap extend penalty of 4, and a frameshift gap penalty of 5.

The percent identity between two amino acid or nucleotide sequences can be determined using the algorithm of E. Meyers and W. Miller ((1989) CABIOS, 4:11-17) which has been incorporated into the ALIGN program (version 2.0), using a PAM120 weight residue table, a gap length penalty of 12 and a gap penalty of 4.

The nucleic acid and protein sequences described herein can be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to 47476, 67210, 49875, 46842, 33201, 83378, 84233, 64708, 85041, or 84234 nucleic acid molecules of the invention. BLAST protein searches can be performed with the XBLAST program, score=50, wordlength=3 to obtain amino acid sequences homologous to 47476, 67210, 49875, 46842, 33201, 83378, 84233, 64708, 85041, or 84234 protein molecules of the invention. To obtain gapped alignments for comparison purposes, Gapped BLAST can be utilized as described in Altschul et al., (1997) Nucleic Acids Res. 25:3389-3402. When utilizing BLAST and Gapped BLAST programs, the default parameters of the respective programs (e.g., XBLAST and NBLAST) can be used. See http://www.ncbi.nlm.nih.gov.

Particularly preferred 47476, 67210, 49875, 46842, 33201, 83378, 84233, 64708, 85041, or 84234 polypeptides of the present invention have an amino acid sequence substantially identical to the amino acid sequence of SEQ ID NO:2, SEQ ID NO:5, SEQ ID NO:8, SEQ ID NO:11, SEQ ID NO:14, SEQ ID NO:17, SEQ ID NO:20, SEQ ID NO:23, SEQ ID NO:26, or SEQ ID NO:29. In the context of an amino acid sequence, the term “substantially identical” is used herein to refer to a first amino acid that contains a sufficient or minimum number of amino acid residues that are i) identical to, or ii) conservative substitutions of aligned amino acid residues in a second amino acid sequence such that the first and second amino acid sequences can have a common structural domain and/or common functional activity. For example, amino acid sequences that contain a common structural domain having at least about 60%, or 65% identity, likely 75% identity, more likely 85%, 90%. 91%, 92%, 93%, 94%, 95%, 96%, 97%, 98% or 99% identity to SEQ ID NO:2, SEQ ID NO:5, SEQ ID NO:8, SEQ ID NO:11, SEQ ID NO:14, SEQ ID NO:17, SEQ ID NO:20, SEQ ID NO:23, SEQ ID NO:26, or SEQ ID NO:29 are termed substantially identical.

In the context of nucleotide sequence, the term “substantially identical” is used herein to refer to a first nucleic acid sequence that contains a sufficient or minimum number of nucleotides that are identical to aligned nucleotides in a second nucleic acid sequence such that the first and second nucleotide sequences encode a polypeptide having common functional activity, or encode a common structural polypeptide domain or a common functional polypeptide activity. For example, nucleotide sequences having at least about 60%, or 65% identity, likely 75% identity, more likely 85%, 90%. 91%, 92%, 93%, 94%, 95%, 96%, 97%, 98% or 99% identity to SEQ ID NO:1, SEQ ID NO:4, SEQ ID NO:7, SEQ ID NO:10, SEQ ID NO:13, SEQ ID NO:16, SEQ ID NO:19, SEQ ID NO:22, SEQ ID NO:25, or SEQ ID NO:28, or SEQ ID NO:3, SEQ ID NO:6, SEQ ID NO:9, SEQ ID NO:12, SEQ ID NO:15, SEQ ID NO:18, SEQ ID NO:21, SEQ ID NO:24, SEQ ID NO:27, or SEQ ID NO:30 are termed substantially identical.

“Misexpression or aberrant expression”, as used herein, refers to a non-wildtype pattern of gene expression at the RNA or protein level. It includes: expression at non-wild type levels, i.e., over- or under-expression; a pattern of expression that differs from wild type in terms of the time or stage at which the gene is expressed, e.g., increased or decreased expression (as compared with wild type) at a predetermined developmental period or stage; a pattern of expression that differs from wild type in terms of altered, e.g., increased or decreased, expression (as compared with wild type) in a predetermined cell type or tissue type; a pattern of expression that differs from wild type in terms of the splicing size, translated amino acid sequence, post-transitional modification, or biological activity of the expressed polypeptide; a pattern of expression that differs from wild type in terms of the effect of an environmental stimulus or extracellular stimulus on expression of the gene, e.g., a pattern of increased or decreased expression (as compared with wild type) in the presence of an increase or decrease in the strength of the stimulus.

“Subject,” as used herein, refers to human and non-human animals. The term “non-human animals” of the invention includes all vertebrates, e.g., mammals, such as non-human primates (particularly higher primates), sheep, dog, rodent (e.g., mouse or rat), guinea pig, goat, pig, cat, rabbits, cow, and non-mammals, such as chickens, amphibians, reptiles, etc. In a preferred embodiment, the subject is a human. In another embodiment, the subject is an experimental animal or animal suitable as a disease model.

A “purified preparation of cells”, as used herein, refers to an in vitro preparation of cells. In the case cells from multicellular organisms (e.g., plants and animals), a purified preparation of cells is a subset of cells obtained from the organism, not the entire intact organism. In the case of unicellular microorganisms (e.g., cultured cells and microbial cells), it consists of a preparation of at least 10% and more preferably 50% of the subject cells.

Various aspects of the invention are described in further detail below.

Isolated Nucleic Acid Molecules of 47476, 67210, 49875, 46842, 33201, 83378, 84233, 64708, 85041, and 84234

In one aspect, the invention provides, an isolated or purified, nucleic acid molecule that encodes a 47476, 67210, 49875, 46842, 33201, 83378, 84233, 64708, 85041, or 84234 polypeptide described herein, e.g., a full-length 47476, 67210, 49875, 46842, 33201, 83378, 84233, 64708, 85041, or 84234 protein or a fragment thereof, e.g., a biologically active portion of 47476, 67210, 49875, 46842, 33201, 83378, 84233, 64708, 85041, or 84234 protein. Also included is a nucleic acid fragment suitable for use as a hybridization probe, which can be used, e.g., to identify a nucleic acid molecule encoding a polypeptide of the invention, 47476, 67210, 49875, 46842, 33201, 83378, 84233, 64708, 85041, or 84234 mRNA, and fragments suitable for use as primers, e.g., PCR primers for the amplification or mutation of nucleic acid molecules.

In one embodiment, an isolated nucleic acid molecule of the invention includes the nucleotide sequence shown in SEQ ID NO:1, SEQ ID NO:4, SEQ ID NO:7, SEQ ID NO:10, SEQ ID NO:13, SEQ ID NO:16, SEQ ID NO:19, SEQ ID NO:22, SEQ ID NO:25, and SEQ ID NO:28, or a portion of any of these nucleotide sequences. In one embodiment, the nucleic acid molecule includes sequences encoding the human 47476, 67210, 49875, 46842, 33201, 83378, 84233, 64708, 85041, or 84234 protein (i.e., “the coding region” of SEQ ID NO:1, SEQ ID NO:4, SEQ ID NO:7, SEQ ID NO:10, SEQ ID NO:13, SEQ ID NO:16, SEQ ID NO:19, SEQ ID NO:22, SEQ ID NO:25, and SEQ ID NO:28, as shown in SEQ ID NO:3, SEQ ID NO:6, SEQ ID NO:9, SEQ ID NO:12, SEQ ID NO:15, SEQ ID NO:18, SEQ ID NO:21, SEQ ID NO:24, SEQ ID NO:27, or SEQ ID NO:30), as well as 5′ untranslated sequences. Alternatively, the nucleic acid molecule can include only the coding region of SEQ ID NO:1, SEQ ID NO:4, SEQ ID NO:7, SEQ ID NO:10, SEQ ID NO:13, SEQ ID NO:16, SEQ ID NO:19, SEQ ID NO:22, SEQ ID NO:25, and SEQ ID NO:28 (e.g., SEQ ID NO:3, SEQ ID NO:6, SEQ ID NO:9, SEQ ID NO:12, SEQ ID NO:15, SEQ ID NO:18, SEQ ID NO:21, SEQ ID NO:24, SEQ ID NO:27, or SEQ ID NO:30) and, e.g., no flanking sequences which normally accompany the subject sequence. In another embodiment, the nucleic acid molecule encodes a sequence corresponding to a mature fragment of a 47476, 67210, 49875, 46842, 33201, 83378, 84233, 64708, 85041, or 84234 protein.

In another embodiment, an isolated nucleic acid molecule of the invention includes a nucleic acid molecule which is a complement (e.g., a full complement) of the nucleotide sequence shown in SEQ ID NO:1, SEQ ID NO:4, SEQ ID NO:7, SEQ ID NO:10, SEQ ID NO:13, SEQ ID NO:16, SEQ ID NO:19, SEQ ID NO:22, SEQ ID NO:25, or SEQ ID NO:28, or SEQ ID NO:3, SEQ ID NO:6, SEQ ID NO:9, SEQ ID NO:12, SEQ ID NO:15, SEQ ID NO:18, SEQ ID NO:21, SEQ ID NO:24, SEQ ID NO:27, or SEQ ID NO:30, or a portion of any of these nucleotide sequences. In other embodiments, the nucleic acid molecule of the invention is sufficiently complementary to the nucleotide sequence shown in SEQ ID NO:1, SEQ ID NO:4, SEQ ID NO:7, SEQ ID NO:10, SEQ ID NO:13, SEQ ID NO:16, SEQ ID NO:19, SEQ ID NO:22, SEQ ID NO:25, or SEQ ID NO:28, or SEQ ID NO:3, SEQ ID NO:6, SEQ ID NO:9, SEQ ID NO:12, SEQ ID NO:15, SEQ ID NO:18, SEQ ID NO:21, SEQ ID NO:24, SEQ ID NO:27, or SEQ ID NO:30, such that it can hybridize (e.g., under a stringency condition described herein) to the nucleotide sequence shown in SEQ ID NO:1, SEQ ID NO:4, SEQ ID NO:7, SEQ ID NO:10, SEQ ID NO:13, SEQ ID NO:16, SEQ ID NO:19, SEQ ID NO:22, SEQ ID NO:25, or SEQ ID NO:28, or SEQ ID NO:3, SEQ ID NO:6, SEQ ID NO:9, SEQ ID NO:12, SEQ ID NO:15, SEQ ID NO:18, SEQ ID NO:21, SEQ ID NO:24, SEQ ID NO:27, or SEQ ID NO:30, thereby forming a stable duplex.

In one embodiment, an isolated nucleic acid molecule of the present invention includes a nucleotide sequence which is at least about: 60%, 65%, 70%, 75%, 80%, 85%, 90%, 91%, 92%, 93%, 94%, 95%, 96%, 97%, 98%, 99%, or more homologous to the entire length of the nucleotide sequence shown in SEQ ID NO:1, SEQ ID NO:4, SEQ ID NO:7, SEQ ID NO:10, SEQ ID NO:13, SEQ ID NO:16, SEQ ID NO:19, SEQ ID NO:22, SEQ ID NO:25, or SEQ ID NO:28, or SEQ ID NO:3, SEQ ID NO:6, SEQ ID NO:9, SEQ ID NO:12, SEQ ID NO:15, SEQ ID NO:18, SEQ ID NO:21, SEQ ID NO:24, SEQ ID NO:27, or SEQ ID NO:30, or a portion, preferably of the same length, of any of these nucleotide sequences.

47476 Nucleic Acid Fragments

A nucleic acid molecule of the invention can include only a portion of the nucleic acid sequence of SEQ ID NO:1 or SEQ ID NO:3. For example, such a nucleic acid molecule can include a fragment which can be used as a probe or primer or a fragment encoding a portion of a 47476 protein, e.g., an immunogenic or biologically active portion of a 47476 protein. A fragment can comprise those nucleotides of SEQ ID NO:1, which encode a ras guanine nucleotide dissociation stimulator domain of human 47476. The nucleotide sequence determined from the cloning of the 47476 gene allows for the generation of probes and primers designed for use in identifying and/or cloning other 47476 family members, or fragments thereof, as well as 47476 homologues, or fragments thereof, from other species.

In another embodiment, a nucleic acid includes a nucleotide sequence that includes part, or all, of the coding region and extends into either (or both) the 5′ or 3′ noncoding region. Other embodiments include a fragment that includes a nucleotide sequence encoding an amino acid fragment described herein. Nucleic acid fragments can encode a specific domain or site described herein or fragments thereof, particularly fragments thereof which are at least 100 amino acids in length. Preferably, the nucleic acid fragments encode a specific domain or fragment thereof, wherein the domain or fragment is at least 25, 28, 45, 50, 150, 180 and 230 amino acids in length. Fragments also include nucleic acid sequences corresponding to specific amino acid sequences described above or fragments thereof. Nucleic acid fragments should not to be construed as encompassing those fragments that may have been disclosed prior to the invention.

A nucleic acid fragment can include a sequence corresponding to a domain, region, or functional site described herein. A nucleic acid fragment can also include one or more domains, regions, or functional sites described herein. Thus, for example, a 47476 nucleic acid fragment can include a sequence corresponding to a ras guanine nucleotide dissociation stimulator domain, a guanine nucleotide dissociation stimulator domain N-terminal motif, an EF-hand calcium-binding domain, or a phorbol ester/diacylglycerol binding domain (C1 domain).

47476 probes and primers are provided. Typically a probe/primer is an isolated or purified oligonucleotide. The oligonucleotide typically includes a region of nucleotide sequence that hybridizes under a stringency condition described herein to at least about 7, 12 or 15, preferably about 20 or 25, more preferably about 30, 35, 40, 45, 50, 55, 60, 65, or 75 consecutive nucleotides of a sense or antisense sequence of SEQ ID NO:1 or SEQ ID NO:3, or of a naturally occurring allelic variant or mutant of SEQ ID NO:1 or SEQ ID NO:3. Preferably, an oligonucleotide is less than about 200, 150, 120, or 100 nucleotides in length.

In one embodiment, the probe or primer is attached to a solid support, e.g., a solid support described herein.

One exemplary kit of primers includes a forward primer that anneals to the coding strand and a reverse primer that anneals to the non-coding strand. The forward primer can anneal to the start codon, e.g., the nucleic acid sequence encoding amino acid residue 1 of SEQ ID NO:2. The reverse primer can anneal to the ultimate codon, e.g., the codon immediately before the stop codon, e.g., the codon encoding amino acid residue 673 of SEQ ID NO:2. In a preferred embodiment, the annealing temperatures of the forward and reverse primers differ by no more than 5, 4, 3, or 2° C.

In a preferred embodiment, the nucleic acid is a probe which is at least 10, 12, 15, 18, 20 and less than 200, more preferably less than 100, or less than 50, nucleotides in length. It should be identical, or differ by 1, or 2, or less than 5 or 10 nucleotides, from a sequence disclosed herein. If alignment is needed for this comparison the sequences should be aligned for maximum homology. “Looped” out sequences from deletions or insertions, or mismatches, are considered differences.

A probe or primer can be derived from the sense or anti-sense strand of a nucleic acid that encodes: a guanine nucleotide dissociation stimulator domain, e.g., located at about amino acid residues 195 to 381 of SEQ ID NO:2; a guanine nucleotide dissociation stimulator domain N-terminal motif, e.g., located at about amino acid residues 55 to 172 of SEQ ID NO:2; an EF-hand calcium-binding domain, e.g., located at about amino acid residues 470 to 498 of SEQ ID NO:2; or a phorbol ester/diacylglycerol binding domain (C1 domain), e.g., located at about amino acid residues 541 to 590 of SEQ ID NO:2.

In another embodiment a set of primers is provided, e.g., primers suitable for use in a PCR, which can be used to amplify a selected region of a 47476 sequence, e.g., a domain, region, site or other sequence described herein. The primers should be at least 5, 10, or 50 base pairs in length and less than 100, or less than 200, base pairs in length. The primers should be identical, or differs by one base from a sequence disclosed herein or from a naturally occurring variant. For example, primers suitable for amplifying all or a portion of any of the following regions are provided: a ras guanine nucleotide dissociation stimulator domain, e.g., located at about amino acid 195 to 381 of SEQ ID NO:2; a guanine nucleotide dissociation stimulator domain N-terminal motif, e.g., located at about amino acid residues 55 to 172 of SEQ ID NO:2; an EF-hand calcium-binding domain, e.g., located at about amino acid 470 to 498 of SEQ ID NO:2; or a phorbol ester/diacylglycerol binding domain (C1 domain), e.g., located at about amino acid 541 to 590 of SEQ ID NO:2.

A nucleic acid fragment can encode an epitope bearing region of a polypeptide described herein.

A nucleic acid fragment encoding a “biologically active portion of a 47476 polypeptide” can be prepared by isolating a portion of the nucleotide sequence of SEQ ID NO:1 or SEQ ID NO:3, which encodes a polypeptide having a 47476 biological activity (e.g., the biological activities of the 47476 proteins are described herein), expressing the encoded portion of the 47476 protein (e.g., by recombinant expression in vitro) and assessing the activity of the encoded portion of the 47476 protein. For example, a nucleic acid fragment encoding a biologically active portion of 47476 includes a Ras guanine nucleotide dissociation stimulator domain, a guanine nucleotide dissociation stimulator domain N-terminal motif, an EF-handed calcium-binding domain, or a phorbol ester/diacylglycerol binding domain (C1 domain), e.g., about amino acid residues 195 to 381, 55 to 172, 470 to 498, and 541 to 590 of SEQ ID NO:2, respectively. A nucleic acid fragment encoding a biologically active portion of a 47476 polypeptide, may comprise a nucleotide sequence which is greater than 300 or more nucleotides in length In preferred embodiments, a nucleic acid fragment includes a nucleotide sequence which is about 300, 400, 500, 600, 700, 800, 900, 1000, 1100, 1200, 1300 or more nucleotides in length and hybridizes under a stringency condition described herein to a nucleic acid molecule of SEQ ID NO:1, SEQ ID NO:3, or the nucleotide sequence of the DNA insert of the plasmid deposited with ATCC Accession Number as described herein.

In a preferred embodiment, a nucleic acid fragment differs by at least 1, 2, 3, 10, 20, or more nucleotides from a sequence previously disclosed. Differences can include differing in length or sequence identity. For example, a nucleic acid fragment can: include one or more nucleotides from SEQ ID NO:1 or SEQ ID NO:3 located outside a region of overlap with a sequence previously disclosed; not include all of the nucleotides of a sequence previously disclosed, e.g., can be one or more nucleotides shorter (at one or both ends) than the previously disclosed sequence; or can differ by one or more nucleotides in the region of overlap. The nucleic acid fragment can include a sequence identical to the region of nucleotides 1 to 300, 300 to 800, 500 to 1000, 700 to 1300, 1000 to 1500, 1400 to 1900, 1800 to 2300, 2200 to 2700, 2600 to 3100 of SEQ ID NO:1.

67210 Nucleic Acid Fragments

A nucleic acid molecule of the invention can include only a portion of the nucleic acid sequence of SEQ ID NO:4 or SEQ ID NO:6. For example, such a nucleic acid molecule can include a fragment which can be used as a probe or primer or a fragment encoding a portion of a 67210 protein, e.g., an immunogenic or biologically active portion of a 67210 protein. A fragment can comprise those nucleotides of SEQ ID NO:4, which encode a glycosyltransferase domain of human 67210. The nucleotide sequence determined from the cloning of the 67210 gene allows for the generation of probes and primers designed for use in identifying and/or cloning other 67210 family members, or fragments thereof, as well as 67210 homologues, or fragments thereof, from other species.

In another embodiment, a nucleic acid includes a nucleotide sequence that includes part, or all, of the coding region and extends into either (or both) the 5′ or 3′ noncoding region. Other embodiments include a fragment which includes a nucleotide sequence encoding an amino acid fragment described herein. Nucleic acid fragments can encode a specific domain or site described herein or fragments thereof, particularly fragments thereof which are at least 50 amino acids in length, preferably 75, 100, 150, 200, 250, 300, 325, 340 or more amino acids in length. Preferably, the nucleic acid fragments encode a specific domain or fragment thereof, wherein the domain or fragment is at least 200, 300, 400, 500, 600, 700, 800, 900, 1000, 1100, 1200, 1300, 1400, 1500, 1600, 1700 nucleic acids in length. Fragments also include nucleic acid sequences corresponding to specific amino acid sequences described above or fragments thereof. Nucleic acid fragments should not to be construed as encompassing those fragments that may have been disclosed prior to the invention.

A nucleic acid fragment can include a sequence corresponding to a domain, region, or functional site described herein. A nucleic acid fragment can also include one or more domain, region, or functional site described herein. Thus, for example, a 67210 nucleic acid fragment can include a sequence corresponding to a glycosyltransferase domain.

67210 probes and primers are provided. Typically a probe/primer is an isolated or purified oligonucleotide. The oligonucleotide typically includes a region of nucleotide sequence that hybridizes under a stringency condition described herein to at least about 7, 12 or 15, preferably about 20 or 25, more preferably about 30, 35, 40, 45, 50, 55, 60, 65, or 75 consecutive nucleotides of a sense or antisense sequence of SEQ ID NO:4 or SEQ ID NO:6, or of a naturally occurring allelic variant or mutant of SEQ ID NO:4 or SEQ ID NO:6. Preferably, an oligonucleotide is less than about 200, 150, 120, or 100 nucleotides in length.

In one embodiment, the probe or primer is attached to a solid support, e.g., a solid support described herein.

One exemplary kit of primers includes a forward primer that anneals to the coding strand and a reverse primer that anneals to the non-coding strand. The forward primer can anneal to the start codon, e.g., the nucleic acid sequence encoding amino acid residue 1 of SEQ ID NO:5. The reverse primer can anneal to the ultimate codon, e.g., the codon immediately before the stop codon, e.g., the codon encoding amino acid residue 349 of SEQ ID NO:5. In a preferred embodiment, the annealing temperatures of the forward and reverse primers differ by no more than 5, 4, 3, or 2° C.

In a preferred embodiment the nucleic acid is a probe which is at least 10, 12, 15, 18, 20 and less than 200, more preferably less than 100, or less than 50, nucleotides in length. It should be identical, or differ by 1, or 2, or less than 5 or 10 nucleotides, from a sequence disclosed herein. If alignment is needed for this comparison the sequences should be aligned for maximum homology. “Looped” out sequences from deletions or insertions, or mismatches, are considered differences.

A probe or primer can be derived from the sense or anti-sense strand of a nucleic acid which encodes a glycosyltransferase domain (from amino acid 63 to 340 of SEQ ID NO:5).

In another embodiment a set of primers is provided, e.g., primers suitable for use in a PCR, which can be used to amplify a selected region of a 67210 sequence, e.g., a domain, region, site or other sequence described herein. The primers should be at least 5, 10, or 50 base pairs in length and less than 100, or less than 200, base pairs in length. The primers should be identical, or differs by one base from a sequence disclosed herein or from a naturally occurring variant. For example, primers suitable for amplifying all or a portion of any of the following regions are provided: a glycosyltransferase domain, e.g., located at about amino acid 63 to 340 of SEQ ID NO:5.

A nucleic acid fragment can encode an epitope bearing region of a polypeptide described herein.

A nucleic acid fragment encoding a “biologically active portion of a 67210 polypeptide” can be prepared by isolating a portion of the nucleotide sequence of SEQ ID NO:4 or 6, which encodes a polypeptide having a 67210 biological activity (e.g., the biological activities of the 67210 proteins are described herein), expressing the encoded portion of the 67210 protein (e.g., by recombinant expression in vitro) and assessing the activity of the encoded portion of the 67210 protein. For example, a nucleic acid fragment encoding a biologically active portion of 67210 includes a glycosyltransferase domain, e.g., amino acid residues about 63 to 340 of SEQ ID NO:5. A nucleic acid fragment encoding a biologically active portion of a 67210 polypeptide, may comprise a nucleotide sequence which is greater than 200 or more nucleotides in length.

In preferred embodiments, a nucleic acid includes a nucleotide sequence which is about 300, 400, 500, 600, 700, 800, 900, 1000, 1100, 1200, 1300 or more nucleotides in length and hybridizes under a stringency condition described herein to a nucleic acid molecule of SEQ ID NO:4, or SEQ ID NO:6.

In a preferred embodiment, a nucleic acid fragment differs by at least 1, 2, 3, 10, 20, or more nucleotides from the sequence of Genbank accession number AC013776 or AC023550. Differences can include differing in length or sequence identity. For example, a nucleic acid fragment can: include one or more nucleotides from SEQ ID NO:4 or SEQ ID NO:6 located outside the region of nucleotides 290 to 560, 675 to 1042, or 1152 to 1743 of SEQ ID NO:4; not include all of the nucleotides of Genbank accession number AC013776 or AC023550, e.g., can be one or more nucleotides shorter (at one or both ends) than the sequence of Genbank accession number AC013776 or AC023550; or can differ by one or more nucleotides in the region of overlap.

49875 Nucleic Acid Fragments

A nucleic acid molecule of the invention can include only a portion of the nucleic acid sequence of SEQ ID NO:7 or SEQ ID NO:9. For example, such a nucleic acid molecule can include a fragment which can be used as a probe or primer or a fragment encoding a portion of a 49875 protein, e.g., an immunogenic or biologically active portion of a 49875 protein. A fragment can comprise those nucleotides of SEQ ID NO:7, which encode a DEAD type helicase domain of human 49875. The nucleotide sequence determined from the cloning of the 49875 gene allows for the generation of probes and primers designed for use in identifying and/or cloning other 49875 family members, or fragments thereof, as well as 49875 homologues, or fragments thereof, from other species.

In another embodiment, a nucleic acid includes a nucleotide sequence that includes part, or all, of the coding region and extends into either (or both) the 5′ or 3′ noncoding region. Other embodiments include a fragment which includes a nucleotide sequence encoding an amino acid fragment described herein. Nucleic acid fragments can encode a specific domain or site described herein or fragments thereof, particularly fragments thereof which are at least 50 amino acids in length, preferably 100, 200, 250, 300, 350, 400, 450, 500, 550 or more amino acids in length. Fragments also include nucleic acid sequences corresponding to specific amino acid sequences described above or fragments thereof. Nucleic acid fragments should not to be construed as encompassing those fragments that may have been disclosed prior to the invention.

A nucleic acid fragment can include a sequence corresponding to a domain, region, or functional site described herein. A nucleic acid fragment can also include one or more domains, regions, or functional sites described herein. Thus, for example, a 49875 nucleic acid fragment can include a sequence corresponding to a DEAD-type helicase domain (amino acid residues 22 to 245 of SEQ ID NO:8); a DEAD-box subfamily ATP-dependent helicase signature motif (amino acid 169 to 177 of SEQ ID NO:8); a conserved helicase C-terminal domain (amino acid residues 281 to 363 of SEQ ID NO:8); an ATP/GTP-binding site motif A (P-loop) (amino acid 53 to 60 of SEQ ID NO:8).

49875 probes and primers are provided. Typically a probe/primer is an isolated or purified oligonucleotide. The oligonucleotide typically includes a region of nucleotide sequence that hybridizes under a stringency condition described herein to at least about 7, 12 or 15, preferably about 20 or 25, more preferably about 30, 35, 40, 45, 50, 55, 60, 65, or 75 consecutive nucleotides of a sense or antisense sequence of SEQ ID NO:7 or SEQ ID NO:9, or of a naturally occurring allelic variant or mutant of SEQ ID NO:7 or SEQ ID NO:9. Preferably, an oligonucleotide is less than about 200, 150, 120, or 100 nucleotides in length.

In one embodiment, the probe or primer is attached to a solid support, e.g., a solid support described herein.

One exemplary kit of primers includes a forward primer that anneals to the coding strand and a reverse primer that anneals to the non-coding strand. The forward primer can anneal to the start codon, e.g., the nucleic acid sequence encoding amino acid residue 1 of SEQ ID NO:8. The reverse primer can anneal to the ultimate codon, e.g., the codon immediately before the stop codon, e.g., the codon encoding amino acid residue 600 of SEQ ID NO:8. In a preferred embodiment, the annealing temperatures of the forward and reverse primers differ by no more than 5, 4, 3, or 2° C.

In a preferred embodiment the nucleic acid is a probe which is at least 10, 12, 15, 18, 20 and less than 200, more preferably less than 100, or less than 50, nucleotides in length. It should be identical, or differ by 1, or 2, or less than 5 or 10 nucleotides, from a sequence disclosed herein. If alignment is needed for this comparison the sequences should be aligned for maximum homology. “Looped” out sequences from deletions or insertions, or mismatches, are considered differences.

A probe or primer can be derived from the sense or anti-sense strand of a nucleic acid which encodes: a DEAD-type helicase domain, e.g, about amino acid residues 22 to 245 of SEQ ID NO:8; a DEAD-box subfamily ATP-dependent helicase signature motif, e.g., about amino acid residues 169 to 177 of SEQ ID NO:8; a conserved helicase C-terminal domain, e.g., about amino acid residues 281 to 363 of SEQ ID NO:8; or an ATP/GTP-binding site motif A (P-loop), e.g., about amino acid residues 53 to 60 of SEQ ID NO:8.

In another embodiment a set of primers is provided, e.g., primers suitable for use in a PCR, which can be used to amplify a selected region of a 49875 sequence, e.g., a domain, region, site or other sequence described herein. The primers should be at least 5, 10, or 50 base pairs in length and less than 100, or less than 200, base pairs in length. The primers should be identical, or differs by one base from a sequence disclosed herein or from a naturally occurring variant. For example, primers suitable for amplifying all or a portion of any of the following regions are provided: a DEAD-type helicase domain, e.g, about amino acid residues 22 to 245 of SEQ ID NO:8; a DEAD-box subfamily ATP-dependent helicase signature motif, e.g., about amino acid residues 169 to 177 of SEQ ID NO:8; a conserved helicase C-terminal domain, e.g., about amino acid residues 281 to 363 of SEQ ID NO:8; or an ATP/GTP-binding site motif A (P-loop), e.g., about amino acid residues 53 to 60 of SEQ ID NO:8.

A nucleic acid fragment can encode an epitope bearing region of a polypeptide described herein.

A nucleic acid fragment encoding a “biologically active portion of a 49875 polypeptide” can be prepared by isolating a portion of the nucleotide sequence of SEQ ID NO:7 or SEQ ID NO:9, which encodes a polypeptide having a 49875 biological activity (e.g., the biological activities of the 49875 proteins are described herein), expressing the encoded portion of the 49875 protein (e.g., by recombinant expression in vitro) and assessing the activity of the encoded portion of the 49875 protein. For example, a nucleic acid fragment encoding a biologically active portion of 49875 includes a DEAD type helicase domain, e.g., amino acid residues about 22 to 245 of SEQ ID NO:8. A nucleic acid fragment encoding a biologically active portion of a 49875 polypeptide, may comprise a nucleotide sequence which is greater than 150 or more nucleotides in length.

In preferred embodiments, a nucleic acid includes a nucleotide sequence which is about 300, 400, 500, 600, 700, 800, 900, 1000, 1100, 1200, 1300 or more nucleotides in length and hybridizes under a stringency condition described herein to a nucleic acid molecule of SEQ ID NO:7, or SEQ ID NO:9.

In a preferred embodiment, a nucleic acid fragment differs by at least 1, 2, 3, 10, 20, or more nucleotides from a sequence disclosed in WO 01/55301, WO 01/57188 or WO 01/62927. Differences can include differing in length or sequence identity. For example, a nucleic acid fragment can: include one or more nucleotides from SEQ ID NO:7 or SEQ ID NO:9 located outside a region of overlap with a sequence disclosed in WO 01/55301, WO 01/57188 or WO 01/62927; not include all of the nucleotides of a sequence previously disclosed, e.g., can be one or more nucleotides shorter (at one or both ends) than a sequence disclosed in WO 01/55301, WO 01/57188 or WO 01/62927; or can differ by one or more nucleotides in the region of overlap.

46842 Nucleic Acid Fragments

A nucleic acid molecule of the invention can include only a portion of the nucleic acid sequence of SEQ ID NO:10 or SEQ ID NO:12, or the nucleotide sequence of the DNA insert of the plasmid deposited with ATCC Accession Number as described herein. For example, such a nucleic acid molecule can include a fragment which can be used as a probe or primer or a fragment encoding a portion of a 46842 protein, e.g., an immunogenic or biologically active portion of a 46842 protein. A fragment can comprise those nucleotides of SEQ ID NO:1 which encode an ArfGAP domain of human 46842. The nucleotide sequence determined from the cloning of the 46842 gene allows for the generation of probes and primers designed for use in identifying and/or cloning other 46842 family members, or fragments thereof, as well as 46842 homologues, or fragments thereof, from other species.

In another embodiment, a nucleic acid includes a nucleotide sequence that includes part, or all, of the coding region and extends into either (or both) the 5′ or 3′ noncoding region. Other embodiments include a fragment which includes a nucleotide sequence encoding an amino acid fragment described herein. Nucleic acid fragments can encode a specific domain or site described herein or fragments thereof, particularly fragments thereof which are at least 100, 200, 300, 400, or 500 amino acids in length. Fragments also include nucleic acid sequences corresponding to specific amino acid sequences described above or fragments thereof. Nucleic acid fragments should not to be construed as encompassing those fragments that may have been disclosed prior to the invention.

A nucleic acid fragment can include a sequence corresponding to a domain, region, or functional site described herein. A nucleic acid fragment can also include one or more domain, region, or functional site described herein. Thus, for example, a 46842 nucleic acid fragment can include a sequence corresponding to an ArfGAP domain.

46842 probes and primers are provided. Typically a probe/primer is an isolated or purified oligonucleotide. The oligonucleotide typically includes a region of nucleotide sequence that hybridizes under a stringency condition described herein to at least about 7, 12 or 15, preferably about 20 or 25, more preferably about 30, 35, 40, 45, 50, 55, 60, 65, or 75 consecutive nucleotides of a sense or antisense sequence of SEQ ID NO:10 or SEQ ID NO:12, or of a naturally occurring allelic variant or mutant of SEQ ID NO:10 or SEQ ID NO:12. Preferably, an oligonucleotide is less than about 200, 150, 120, or 100 nucleotides in length.

In one embodiment, the probe or primer is attached to a solid support, e.g., a solid support described herein.

One exemplary kit of primers includes a forward primer that anneals to the coding strand and a reverse primer that anneals to the non-coding strand. The forward primer can anneal to the start codon, e.g., the nucleic acid sequence encoding amino acid residue 1 of SEQ ID NO:11. The reverse primer can anneal to the ultimate codon, e.g., the codon immediately before the stop codon, e.g., the codon encoding amino acid residue 834 of SEQ ID NO:11. In a preferred embodiment, the annealing temperatures of the forward and reverse primers differ by no more than 5, 4, 3, or 2° C.

In a preferred embodiment the nucleic acid is a probe which is at least 10, 12, 15, 18, 20 and less than 200, more preferably less than 100, or less than 50, nucleotides in length. It should be identical, or differ by 1, or 2, or less than 5 or 10 nucleotides, from a sequence disclosed herein. If alignment is needed for this comparison the sequences should be aligned for maximum homology. “Looped” out sequences from deletions or insertions, or mismatches, are considered differences.

A probe or primer can be derived from the sense or anti-sense strand of a nucleic acid which encodes: a PH domain, e.g., located at about amino acid residues 269 to 363 SEQ ID NO:11; an ArfGAP domain, e.g., located at about amino acid residues 403 to 525 of SEQ ID NO:11; or an ankyrin repeat domain, e.g., located at about amino acid residues 702 to 734 or 735 to 767 of SEQ ID NO:11.

In another embodiment a set of primers is provided, e.g., primers suitable for use in a PCR, which can be used to amplify a selected region of a 46842 sequence, e.g., a domain, region, site or other sequence described herein. The primers should be at least 5, 10, or 50 base pairs in length and less than 100, or less than 200, base pairs in length. The primers should be identical, or differs by one base from a sequence disclosed herein or from a naturally occurring variant. For example, primers suitable for amplifying all or a portion of any of the following regions are provided: a PH domain, e.g., located at about amino acid residues 269 to 363 SEQ ID NO:11; an ArfGAP domain, e.g., located at about amino acid residues 403 to 525 of SEQ ID NO:11; or an ankyrin repeat domain, e.g., located at about amino acid residues 702 to 734 or 735 to 767 of SEQ ID NO:11.

A nucleic acid fragment can encode an epitope bearing region of a polypeptide described herein.

A nucleic acid fragment encoding a “biologically active portion of a 46842 polypeptide” can be prepared by isolating a portion of the nucleotide sequence of SEQ ID NO:10 or SEQ ID NO:13, or the nucleotide sequence of the DNA insert of the plasmid deposited with ATCC Accession Number as disclosed herein, which encodes a polypeptide having a 46842 biological activity (e.g., the biological activities of the 46842 proteins are described herein), expressing the encoded portion of the 46842 protein (e.g., by recombinant expression in vitro) and assessing the activity of the encoded portion of the 46842 protein. For example, a nucleic acid fragment encoding a biologically active portion of 46842 includes an ArfGAP domain, e.g., about amino acid residues 403 to 525 of SEQ ID NO:11. A nucleic acid fragment encoding a biologically active portion of a 46842 polypeptide may comprise a nucleotide sequence which is greater than 300 or more nucleotides in length.

In preferred embodiments, a nucleic acid includes a nucleotide sequence which is about 300, 400, 500, 600, 700, 800, 900, 1000, 1200, 1400, 1800 or more nucleotides in length and hybridizes under a stringency condition described herein to a nucleic acid molecule of SEQ ID NO:10 or SEQ ID NO:12.

In a preferred embodiment, a nucleic acid fragment differs by at least 1, 2, 3, 10, 20, or more nucleotides from the sequence of GenBank accession number BAB21807 (KIAA1716). Differences can include differing in length or sequence identity. For example, a nucleic acid fragment can: include one or more nucleotides from SEQ ID NO:10 or SEQ ID NO:12 located outside the region of nucleotides 1 to 100, 50 to 109, 200 to 400, 600 to 800, 700 to 1200, 1200 to 1500, 1420 to 1950, or 2210 to 2425; not include all of the nucleotides of GenBank accession number BAB21807; or can differ by one or more nucleotides in the region of overlap:

33201 Nucleic Acid Fragments

A nucleic acid molecule of the invention can include only a portion of the nucleic acid sequence of SEQ ID NO:13 or SEQ ID NO:15. For example, such a nucleic acid molecule can include a fragment which can be used as a probe or primer or a fragment encoding a portion of a 33201 protein, e.g., an immunogenic or biologically active portion of a 33201 protein. A fragment can comprise those nucleotides of SEQ ID NO:13, which encode a dehydrogenase/reductase domain of human 33201. The nucleotide sequence determined from the cloning of the 33201 gene allows for the generation of probes and primers designed for use in identifying and/or cloning other 33201 family members, or fragments thereof, as well as 33201 homologues, or fragments thereof, from other species.

In another embodiment, a nucleic acid includes a nucleotide sequence that includes part, or all, of the coding region and extends into either (or both) the 5′ or 3′ noncoding region. Other embodiments include a fragment which includes a nucleotide sequence encoding an amino acid fragment described herein. Nucleic acid fragments can encode a specific domain or site described herein or fragments thereof, particularly fragments thereof which are at least 100 amino acids in length. Preferably, the nucleic acid fragments encode a specific domain or fragment thereof, wherein the domain or fragment is at least 105, or more preferably 110, 120, or even 130 amino acids in length. Fragments also include nucleic acid sequences corresponding to specific amino acid sequences described above or fragments thereof. Nucleic acid fragments should not to be construed as encompassing those fragments that may have been disclosed prior to the invention.

A nucleic acid fragment can include a sequence corresponding to a domain, region, or functional site described herein. A nucleic acid fragment can also include one or more domains, regions, or functional sites described herein. Thus, for example, a 33201 nucleic acid fragment can include a sequence corresponding to a dehydrogenase/reductase activity.

33201 probes and primers are provided. Typically a probe/primer is an isolated or purified oligonucleotide. The oligonucleotide typically includes a region of nucleotide sequence that hybridizes under a stringency condition described herein to at least about 7, 12 or 15, preferably about 20 or 25, more preferably about 30, 35, 40, 45, 50, 55, 60, 65, or 75 consecutive nucleotides of a sense or antisense sequence of SEQ ID NO:13 or SEQ ID NO:15, or of a naturally occurring allelic variant or mutant of SEQ ID NO:13 or SEQ ID NO:15. Preferably, an oligonucleotide is less than about 200, 150, 120, or 100 nucleotides in length.

In one embodiment, the probe or primer is attached to a solid support, e.g., a solid support described herein.

One exemplary kit of primers includes a forward primer that anneals to the coding strand and a reverse primer that anneals to the non-coding strand. The forward primer can anneal to the start codon, e.g., the nucleic acid sequence encoding amino acid residue 1 of SEQ ID NO:14. The reverse primer can anneal to the ultimate codon, e.g., the codon immediately before the stop codon, e.g., the codon encoding amino acid residue 351 of SEQ ID NO:14. In a preferred embodiment, the annealing temperatures of the forward and reverse primers differ by no more than 5, 4, 3, or 2° C.

In a preferred embodiment the nucleic acid is a probe which is at least 10, 12, 15, 18, 20 and less than 200, more preferably less than 100, or less than 50, nucleotides in length. It should be identical, or differ by 1, or 2, or less than 5 or 10 nucleotides, from a sequence disclosed herein. If alignment is needed for this comparison the sequences should be aligned for maximum homology. “Looped” out sequences from deletions or insertions, or mismatches, are considered differences.

A probe or primer can be derived from the sense or anti-sense strand of a nucleic acid which encodes a dehydrogenase/reductase domain (e.g., about amino acid residues 22 to 345 of SEQ ID NO:14).

In another embodiment a set of primers is provided, e.g., primers suitable for use in a PCR, which can be used to amplify a selected region of a 33201 sequence, e.g., a domain, region, site or other sequence described herein. The primers should be at least 5, 10, or 50 base pairs in length and less than 100, or less than 200, base pairs in length. The primers should be identical, or differs by one base from a sequence disclosed herein or from a naturally occurring variant. For example, primers suitable for amplifying all or a portion of any of the following regions are provided: a dehydrogenase/reductase domain, e.g., from about amino acid residues 22 to 345 of SEQ ID NO:14.

A nucleic acid fragment can encode an epitope bearing region of a polypeptide described herein.

A nucleic acid fragment encoding a “biologically active portion of a 33201 polypeptide” can be prepared by isolating a portion of the nucleotide sequence of SEQ ID NO:13 or SEQ ID NO:15, which encodes a polypeptide having a 33201 biological activity (e.g., the biological activities of the 33201 proteins are described herein), expressing the encoded portion of the 33201 protein (e.g., by recombinant expression in vitro) and assessing the activity of the encoded portion of the 33201 protein. For example, a nucleic acid fragment encoding a biologically active portion of 33201 can include a dehydrogenase/reductase domain, e.g., amino acid residues about 22 to 345 of SEQ ID NO:14. A nucleic acid fragment encoding a biologically active portion of a 33201 polypeptide, may comprise a nucleotide sequence which is greater than 300 or more nucleotides in length.

In preferred embodiments, a nucleic acid includes a nucleotide sequence which is about 300, 400, 500, 600, 700, 800, 900, 1000, 1100, 1200, 1300 or more nucleotides in length and hybridizes under a stringency condition described herein to a nucleic acid molecule of SEQ ID NO:13, or SEQ ID NO:15.

In a preferred embodiment, a nucleic acid fragment differs by at least 1, 2, 3, 10, 20, or more nucleotides from the sequence of Genbank accession number AC005520. Differences can include differing in length or sequence identity. For example, a nucleic acid fragment can: include one or more nucleotides from SEQ ID NO:13 or SEQ ID NO:15 located outside the region of nucleotides 1117 to 1423, 1425 to 1627, or 1629 to 1717; not include all of the nucleotides of Genbank accession AC005520, e.g., can be one or more nucleotides shorter (at one or both ends) than the sequence of Genbank accession number AC005520, or can differ by one or more nucleotides in the region of overlap.

83378, 84233, 64708, 85041, or 84234 Nucleic Acid Fragments

A nucleic acid molecule of the invention can include only a portion of the nucleic acid sequence of SEQ ID NO:16, SEQ ID NO:19, SEQ ID NO:22, SEQ ID NO:25, or SEQ ID NO:28, or SEQ ID NO:18, SEQ ID NO:21, SEQ ID NO:24, SEQ ID NO:27, or SEQ ID NO:30. For example, such a nucleic acid molecule can include a fragment which can be used as a probe or primer or a fragment encoding a portion of a 83378, 84233, 64708, 85041, or 84234 protein, e.g., an immunogenic or biologically active portion of a 83378, 84233, 64708, 85041, or 84234 protein. A fragment can comprise those nucleotides of SEQ ID NO:16, SEQ ID NO:19, SEQ ID NO:22, SEQ ID NO:25, or SEQ ID NO:28, which encode a cation efflux domain of human 83378, 84233, 64708, 85041, or 84234. The nucleotide sequence determined from the cloning of the 83378, 84233, 64708, 85041, or 84234 gene allows for the generation of probes and primers designed for use in identifying and/or cloning other 83378, 84233, 64708, 85041, or 84234 family members, or fragments thereof, as well as 83378, 84233, 64708, 85041, or 84234 homologues, or fragments thereof, from other species.

In another embodiment, a nucleic acid includes a nucleotide sequence that includes part, or all, of the coding region and extends into either (or both) the 5′ or 3′ noncoding region. Other embodiments include a fragment which includes a nucleotide sequence encoding an amino acid fragment described herein. Nucleic acid fragments can encode a specific domain or site described herein or fragments thereof, particularly fragments thereof which are at least 50 amino acids in length. Preferably, the nucleic acid fragments encode a specific domain or fragment thereof, wherein the domain or fragment is at least 100, 150, 200, 250, 300, 350, 400, 450, 500, 550, 600, 650, 700, or 750 amino acids in length. Fragments also include nucleic acid sequences corresponding to specific amino acid sequences described above or fragments thereof. Nucleic acid fragments should not to be construed as encompassing those fragments that may have been disclosed prior to the invention.

A nucleic acid fragment can include a sequence corresponding to a domain, region, or functional site described herein. A nucleic acid fragment can also include one or more domain, region, or functional site described herein. Thus, for example, a 83378, 84233, 64708, 85041, or 84234 nucleic acid fragment can include a sequence corresponding to a cation efflux domain, a transmembrane domain, a cytoplasmic domain, or a non-cytoplasmic loop.

83378, 84233, 64708, 85041, or 84234 probes and primers are provided. Typically a probe/primer is an isolated or purified oligonucleotide. The oligonucleotide typically includes a region of nucleotide sequence that hybridizes under a stringency condition described herein to at least about 7, 12 or 15, preferably about 20 or 25, more preferably about 30, 35, 40, 45, 50, 55, 60, 65, or 75 consecutive nucleotides of a sense or antisense sequence of SEQ ID NO:16, SEQ ID NO:18, SEQ ID NO:19, SEQ ID NO:21, SEQ ID NO:22, SEQ ID NO:24, SEQ ID NO:25, SEQ ID NO:27, SEQ ID NO:28, or SEQ ID NO:30, or of a naturally occurring allelic variant or mutant of SEQ ID NO:16, SEQ ID NO:18, SEQ ID NO:19, SEQ ID NO:21, SEQ ID NO:22, SEQ ID NO:24; SEQ ID NO:25, SEQ ID NO:27, SEQ ID NO:28, or SEQ ID NO:30. Preferably, an oligonucleotide is less than about 200, 150, 120, or 100 nucleotides in length.

In one embodiment, the probe or primer is attached to a solid support, e.g., a solid support described herein.

One exemplary kit of primers includes a forward primer that anneals to the coding strand and a reverse primer that anneals to the non-coding strand. The forward primer can anneal to the start codon, e.g., the nucleic acid sequence encoding amino acid residue 1 of SEQ ID NO:17, SEQ ID NO:20, SEQ ID NO:23, SEQ ID NO:26, or SEQ ID NO:29. The reverse primer can anneal to the ultimate codon, e.g., the codon immediately before the stop codon, e.g., the codon encoding amino acid residue 485 of SEQ ID NO:17, amino acid residue 320 of SEQ ID NO:20, amino acid residue 461 of SEQ ID NO:23, amino acid residue 765 of SEQ ID NO:26, or amino acid residue 376 of SEQ ID NO:29. In a preferred embodiment, the annealing temperatures of the forward and reverse primers differ by no more than 5, 4, 3, or 2° C.

In a preferred embodiment the nucleic acid is a probe which is at least 10, 12, 15, 18, 20 and less than 200, more preferably less than 100, or less than 50, nucleotides in length. It should be identical, or differ by 1, or 2, or less than 5 or 10 nucleotides, from a sequence disclosed herein. If alignment is needed for this comparison the sequences should be aligned for maximum homology. “Looped” out sequences from deletions or insertions, or mismatches, are considered differences.

A probe or primer can be derived from the sense or anti-sense strand of a nucleic acid which encodes: a cation efflux domain (e.g., residues 11 to 133 or 231 to 389 of SEQ ID NO:17, residues 25 to 310 of SEQ ID NO:20, residues 55 to 153 or 227 to 320 of SEQ ID NO:23, residues 419 to 733 of SEQ ID NO:26, or residues 38 to 349 of SEQ ID NO:29); a transmembrane domain (e.g., residues 11-31, 44-61, 79-98, 115-134, 241-265, or 283-299 of SEQ ID NO:17; residues 25-49, 58-74,92-113, 128-147, 167-191, or 201-218 of SEQ ID NO:20; residues 34-51,58-82, 101-119, 137-155,202-219, or 232-249 of SEQ ID NO:23; residues 59-77, 99-119, 129-145, 152-168, 190-214, 239-258, 267-288, 304-320, 343-362, 419-439, 486-505, 521-541, 592-613, or 618-641 of SEQ ID NO:26; or residues 38-58, 71-87, 105-123, 141-159, 237-256, or 263-286 of SEQ ID NO:29); a cytoplasmic domain (e.g., residues 1-10, 62-78, 135-240, or 300-485 of SEQ ID NO:17; residues 1-24, 75-91, 148-166, or 219-320 of SEQ ID NO:20; residues 1-33, 83-100, 156-201, or 250-461 of SEQ ID NO:23; residues 1-58, 120-128, 169-189, 259-266, 321-342, 438-485, 542-591, or 642-765 of SEQ ID NO:26; and residues 1-37, 88-104, 160-236, or 287-376 of SEQ ID NO:29); or a non-cytoplasmic loop (residues 32-43, 99-114, or 266-282 of SEQ ID NO:17; residues 50-57, 114-127, or 192-200 of SEQ ID NO:20; residues 52-57, 120-136, or 220-231 of SEQ ID NO:23; residues 78-98, 146-151, 215-238, 289-303, 363-418, 506-520, or 614-617 of SEQ ID NO:26; or residues 59-70, 124-140, or 257-262 of SEQ ID NO:29).

In another embodiment a set of primers is provided, e.g., primers suitable for use in a PCR, which can be used to amplify a selected region of a 83378, 84233, 64708, 85041, or 84234 sequence, e.g., a domain, region, site or other sequence described herein. The primers should be at least 5, 10, or 50 base pairs in length and less than 100, or less than 200, base pairs in length. The primers should be identical, or differs by one base from a sequence disclosed herein or from a naturally occurring variant. For example, primers suitable for amplifying all or a portion of any of the following regions are provided a cation efflux domain (e.g., residues 11 to 133 or 231 to 389 of SEQ ID NO:17, residues 25 to 310 of SEQ ID NO:20, residues 55 to 153 or 227 to 320 of SEQ ID NO:23, residues 419 to 733 of SEQ ID NO:26, or residues 38 to 349 of SEQ ID NO:29); a transmembrane domain (e.g., residues 11-31, 44-61, 79-98, 115-134, 241-265, or 283-299 of SEQ ID NO:17; residues 25-49, 58-74, 92-113, 128-147, 167-191, or 201-218 of SEQ ID NO:20; residues 34-51, 58-82, 101-119, 137-155, 202-219, or 232-249 of SEQ ID NO:23; residues 59-77, 99-119, 129-145, 152-168, 190-214, 239-258, 267-288, 304-320, 343-362, 419-439, 486-505, 521-541, 592-613, or 618-641 of SEQ ID NO:26; or residues 38-58, 71-87, 105-123, 141-159, 237-256, or 263-286 of SEQ ID NO:29); a cytoplasmic domain (e.g., residues 1-10, 62-78, 135-240, or 300-485 of SEQ ID NO:17; residues 1-24, 75-91, 148-166, or 219-320 of SEQ ID NO:20; residues 1-33, 83-100, 156-201, or 250-461 of SEQ ID NO:23; residues 1-58, 120-128, 169-189, 259-266, 321-342, 438-485, 542-591, or 642-765 of SEQ ID NO:26; and residues 1-37, 88-104, 160-236, or 287-376 of SEQ ID NO:29); or a non-cytoplasmic loop (residues 32-43, 99-114, or 266-282 of SEQ ID NO:17; residues 50-57, 114-127, or 192-200 of SEQ ID NO:20; residues 52-57, 120-136, or 220-231 of SEQ ID NO:23; residues 78-98, 146-151, 215-238, 289-303, 363-418, 506-520, or 614-617 of SEQ ID NO:26; or residues 59-70, 124-140, or 257-262 of SEQ ID NO:29).

A nucleic acid fragment can encode an epitope bearing region of a polypeptide described herein.

A nucleic acid fragment encoding a “biologically active portion of a 83378, 84233, 64708, 85041, or 84234 polypeptide” can be prepared by isolating a portion of the nucleotide sequence of SEQ ID NO:16, SEQ ID NO:18, SEQ ID NO:19, SEQ ID NO:21, SEQ ID NO:22, SEQ ID NO:24, SEQ ID NO:25, SEQ ID NO:27, SEQ ID NO:28, or SEQ ID NO:30, which encodes a polypeptide having a 83378, 84233, 64708, 85041, or 84234 biological activity (e.g., the biological activities of the 83378, 84233, 64708, 85041, or 84234 proteins are described herein), expressing the encoded portion of the 83378, 84233, 64708, 85041, or 84234 protein (e.g., by recombinant expression in vitro) and assessing the activity of the encoded portion of the 83378, 84233, 64708, 85041, or 84234 protein. For example, a nucleic acid fragment encoding a biologically active portion of 83378, 84233, 64708, 85041, or 84234 includes a cation efflux domain, e.g., residues 11 to 133 or 231 to 389 of SEQ ID NO:17, residues 25 to 310 of SEQ ID NO:20, residues 55 to 153 or 227 to 320 of SEQ ID NO:23, residues 419 to 733 of SEQ ID NO:26, or residues 38 to 349 of SEQ ID NO:29. A nucleic acid fragment encoding a biologically active portion of a 83378, 84233, 64708, 85041, or 84234 polypeptide, may comprise a nucleotide sequence which is greater than 300 or more nucleotides in length.

In preferred embodiments, a nucleic acid includes a nucleotide sequence which is about 300, 400, 500, 600, 700, 800, 900, 1000, 1100, 1200, 1300, 1400, 1500, 1600, 1700, 1800, or more nucleotides in length and hybridizes under a stringency condition described herein to a nucleic acid molecule of SEQ ID NO:16, SEQ ID NO:18, SEQ ID NO:19, SEQ ID NO:21, SEQ ID NO:22, SEQ ID NO:24, SEQ ID NO:25, SEQ ID NO:27, SEQ ID NO:28, or SEQ ID NO:30.

In a preferred embodiment, a 83378 nucleic acid fragment differs by at least 1, 2, 3, 10, 20, or more nucleotides from the sequence of Genbank accession number AL359609 or a sequence disclosed in WO 01/62918, WO 01/55314, WO 01/55355. Differences can include differing in length or sequence identity. For example, a nucleic acid fragment can: include one or more nucleotides from SEQ ID NO:16 or SEQ ID NO:18 located outside the region of nucleotides 7-1044, 1085-1638, 1253-1638, 1299-1638, 723-1775 of SEQ ID NO:16; not include all of the nucleotides of the sequence of Genbank accession number AL359609 or a sequence disclosed in WO 01/62918, WO 01/55314, WO 01/55355, e.g., can be one or more nucleotides shorter (at one or both ends) than the sequence of Genbank accession number AL359609 or a sequence disclosed in WO 01/62918, WO 01/55314, WO 01/55355; or can differ by one or more nucleotides in the region of overlap.

In a preferred embodiment, a 84233 nucleic acid fragment differs by at least 1, 2, 3, 10, 20, or more nucleotides from the sequence of Genbank accession number AX061210 or AX086187 or a sequence disclosed in WO 01/12659, WO 01/51628, or WO 00/78953. Differences can include differing in length or sequence identity. For example, a nucleic acid fragment can: include one or more nucleotides from SEQ ID NO:19 or SEQ ID NO:21 located outside the region of nucleotides 420-2103, 998-1589, 1-429, 1146-1582, 996-1425, 998-1341, 996-1379, 996-1368 of SEQ ID NO:19; not include all of the nucleotides of Genbank accession number AX061210 or AX086187 or a sequence disclosed in WO 01/12659, WO 01/51628, or WO 00/78953, e.g., can be one or more nucleotides shorter (at one or both ends) than the sequence of Genbank accession number AX061210 or AX086187 or a sequence disclosed in WO 01/12659, WO 01/51628, or WO 00/78953; or can differ by one or more nucleotides in the region of overlap.

In a preferred embodiment, a 64708 nucleic acid fragment differs by at least 1, 2, 3, 10, 20, or more nucleotides from the sequence of Genbank accession number AK000844 or a sequence disclosed in WO 01/57188, WO 01/57270, WO 01/57272, WO 01/57275, WO 01/57276, WO 01/57277, or WO 01/57278. Differences can include differing in length or sequence identity. For example, a nucleic acid fragment can: include one or more nucleotides from SEQ ID NO:22 or SEQ ID NO:24 located outside the region of nucleotides 209-697, 918-1456, 918-1418, 1348-1790, 20-442, 911-1280, or 911-1226 of SEQ ID NO:22; not include all of the nucleotides of Genbank accession number AK000844 or a sequence disclosed in WO 01/57188, WO 01/57270, WO 01/57272, WO 01/57275, WO 01/57276, WO 01/57277, or WO 01/57278, e.g., can be one or more nucleotides shorter (at one or both ends) than the sequence of Genbank accession number AK000844 or a sequence disclosed in WO 01/57188, WO 01/57270, WO 01/57272, WO 01/57275, WO 01/57276, WO 01/57277, or WO 01/57278; or can differ by one or more nucleotides in the region of overlap.

In a preferred embodiment, a 85041 nucleic acid fragment differs by at least 1, 2, 3, 10, 20, or more nucleotides from the sequence of Genbank accession number AK022558, AK022818, or AF233321, or a sequence disclosed in WO 01/40466, WO 01/54472, WO 01/55318, or WO 01/53312. Differences can include differing in length or sequence identity. For example, a nucleic acid fragment can: include one or more nucleotides from SEQ ID NO:25 or SEQ ID NO:27 located outside the region of nucleotides 55-2750, 1069-3010, 1347-3224, 1320-3010,1347-3010, 602-3005, 483-2740, 1015-1942, 786-3259, 602-3005, 883-2051, 2139-3010, 94-809, or 1843-3010 of SEQ ID NO:25; not include all of the nucleotides of Genbank accession number AK022558, AK022818, or AF233321, or a sequence disclosed in WO 01/40466, WO 01/54472, WO 01/55318, or WO 01/53312, e.g., can be one or more nucleotides shorter (at one or both ends) than the sequence of Genbank accession number AK022558, AK022818, or AF233321, or a sequence disclosed in WO 01/40466, WO 01/54472, WO 01/55318, or WO 01/53312; or can differ by one or more nucleotides in the region of overlap.

In a preferred embodiment, a 84234 nucleic acid fragment differs by at least 1, 2, 3, 10, 20, or more nucleotides from a sequence disclosed in WO 01/53312. Differences can include differing in length or sequence identity. For example, a nucleic acid fragment can: include one or more nucleotides from SEQ ID NO:28 or SEQ ID NO:30 located outside the region of nucleotides 129-1292 or 165-1292 of SEQ ID NO:28; not include all of the nucleotides of a sequence disclosed in WO 01/53312, e.g., can be one or more nucleotides shorter (at one or both ends) than the sequence of a sequence disclosed in WO 01/53312; or can differ by one or more nucleotides in the region of overlap.

47476, 67210, 49875, 46842, 33201, 83378, 84233, 64708, 85041, or 84234 Nucleic Acid Variants

The invention further encompasses nucleic acid molecules that differ from the nucleotide sequence shown in SEQ ID NO:1, SEQ ID NO:4, SEQ ID NO:7, SEQ ID NO:10, SEQ ID NO:13, SEQ ID NO:16, SEQ ID NO:19, SEQ ID NO:22, SEQ ID NO:25, or SEQ ID NO:28, or SEQ ID NO:3, SEQ ID NO:6, SEQ ID NO:9, SEQ ID NO:12, SEQ ID NO:15, SEQ ID NO:18, SEQ ID NO:21, SEQ ID NO:24, SEQ ID NO:27, or SEQ ID NO:30. Such differences can be due to degeneracy of the genetic code (and result in a nucleic acid which encodes the same 47476, 67210, 49875, 46842, 33201, 83378, 84233, 64708, 85041, or 84234 proteins as those encoded by the nucleotide sequence disclosed herein. In another embodiment, an isolated nucleic acid molecule of the invention has a nucleotide sequence encoding a protein having an amino acid sequence which differs, by at least 1, but less than 5, 10, 20, 50, or 100 amino acid residues that shown in SEQ ID NO:2, SEQ ID NO:5, SEQ ID NO:8, SEQ ID NO:11, SEQ ID NO:14, SEQ ID NO:17, SEQ ID NO:20, SEQ ID NO:23, SEQ ID NO:26, or SEQ ID NO:29. If alignment is needed for this comparison the sequences should be aligned for maximum homology. The encoded protein can differ by no more than 5, 4, 3, 2, or 1 amino acid. “Looped” out sequences from deletions or insertions, or mismatches, are considered differences.

Nucleic acids of the inventor can be chosen for having codons, which are preferred, or non-preferred, for a particular expression system. E.g., the nucleic acid can be one in which at least one codon, at preferably at least 10%, or 20% of the codons has been altered such that the sequence is optimized for expression in E. coli, yeast, human, insect, or CHO cells.

Nucleic acid variants can be naturally occurring, such as allelic variants (same locus), homologs (different locus), and orthologs (different organism) or can be non naturally occurring. Non-naturally occurring variants can be made by mutagenesis techniques, including those applied to polynucleotides, cells, or organisms. The variants can contain nucleotide substitutions, deletions, inversions and insertions. Variation can occur in either or both the coding and non-coding regions. The variations can produce both conservative and non-conservative amino acid substitutions (as compared in the encoded product).

In a preferred embodiment, the nucleic acid differs from that of SEQ ID NO:1, SEQ ID NO:4, SEQ ID NO:7, SEQ ID NO:10, SEQ ID NO:13, SEQ ID NO:16, SEQ ID NO:19, SEQ ID NO:22, SEQ ID NO:25, or SEQ ID NO:28, or SEQ ID NO:3, SEQ ID NO:6, SEQ ID NO:9, SEQ ID NO:12, SEQ ID NO:15, SEQ ID NO:18, SEQ ID NO:21, SEQ ID NO:24, SEQ ID NO:27, or SEQ ID NO:30, e.g., as follows: by at least one but less than 10, 20, 30, or 40 nucleotides; at least one but less than 1%, 5%, 10% or 20% of the nucleotides in the subject nucleic acid. The nucleic acid can differ by no more than 5, 4, 3, 2, or 1 nucleotide. If necessary for this analysis the sequences should be aligned for maximum homology. “Looped” out sequences from deletions or insertions, or mismatches, are considered differences.

Orthologs, homologs, and allelic variants can be identified using methods known in the art. These variants comprise a nucleotide sequence encoding a polypeptide that is 50%, at least about 55%, typically at least about 70-75%, more typically at least about 80-85%, and most typically at least about 90-95% or more identical to the nucleotide sequence shown in SEQ ID NO:2, SEQ ID NO:5, SEQ ID NO:8, SEQ ID NO:11, SEQ ID NO:14, SEQ ID NO:17, SEQ ID NO:20, SEQ ID NO:23, SEQ ID NO:26, or SEQ ID NO:29, or a fragment of this sequence. Such nucleic acid molecules can readily be identified as being able to hybridize under a stringency condition described herein, to the nucleotide sequence shown in SEQ ID NO:2, SEQ ID NO:5, SEQ ID NO:8, SEQ ID NO:11, SEQ ID NO:14, SEQ ID NO:17, SEQ ID NO:20, SEQ ID NO:23, SEQ ID NO:26, or SEQ ID NO:29, or a fragment of the sequence. Nucleic acid molecules corresponding to orthologs, homologs, and allelic variants of the 47476, 67210, 49875, 46842, 33201, 83378, 84233, 64708, 85041, or 84234 cDNAs of the invention can further be isolated by mapping to the same chromosome or locus as the 47476, 67210, 49875, 46842, 33201, 83378, 84233, 64708, 85041, or 84234 gene.

Preferred 47476 variants include those that are correlated with the ability to exchange guanine nucleotides (GTP for GDP); the ability to bind calcium; the ability to bind at least one and preferably two zinc ions; the ability to bind a second messenger, e.g., diacylglycerol; the ability to bind analogs of diacylglycerol, such as phorbol esters; or the ability to activate the ras superfamily of proteins.

Allelic variants of 47476, e.g., human 47476, include both functional and non-functional proteins. Functional allelic variants are naturally occurring amino acid sequence variants of the 47476 protein within a population that maintain the ability to exchange guanine nucleotides (GTP for GDP); to bind calcium; to bind at least one and preferably two zinc ions; to bind a second messenger, e.g., diacylglycerol; to bind analogs of diacylglycerol, such as phorbol esters; or to activate one or more members of the ras superfamily of proteins. Functional allelic variants will typically contain only conservative substitution of one or more amino acids of SEQ ID NO:2, or substitution, deletion or insertion of non-critical residues in non-critical regions of the protein. Non-functional allelic variants are naturally-occurring amino acid sequence variants of the 47476, e.g., human 47476, protein within a population that do not have the ability to: exchange guanine nucleotides (GTP for GDP); to bind calcium; bind at least one and preferably two zinc ions; bind a second messenger, e.g., diacylglycerol; bind analogs of diacylglycerol, such as phorbol esters; or to activate one or more members of the ras superfamily of proteins. Non-functional allelic variants will typically contain a non-conservative substitution, a deletion, or insertion, or premature truncation of the amino acid sequence of SEQ ID NO:2, or a substitution, insertion, or deletion in critical residues or critical regions of the protein.

Preferred 67210 variants include those that are correlated with glycosyltransferase activity.

Allelic variants of 67210, e.g., human 67210, include both functional and non-functional proteins. Functional allelic variants are naturally occurring amino acid sequence variants of the 67210 protein within a population that maintain the ability to transfer an activated mono- or oligosaccharide residue to an existing acceptor molecule for the initiation or elongation of a carbohydrate chain. Functional allelic variants will typically contain only conservative substitution of one or more amino acids of SEQ ID NO:5, or substitution, deletion or insertion of non-critical residues in non-critical regions of the protein. Non-functional allelic variants are naturally-occurring amino acid sequence variants of the 67210, e.g., human 67210, protein within a population that do not have the ability to transferr an activated mono- or oligosaccharide residue to an existing acceptor molecule for the initiation or elongation of a carbohydrate chain. Non-functional allelic variants will typically contain a non-conservative substitution, a deletion, or insertion, or premature truncation of the amino acid sequence of SEQ ID NO:5, or a substitution, insertion, or deletion in critical residues or critical regions of the protein.

Preferred 49875 variants include those that are correlated with helicase activity, e.g., RNA or DNA helicase activity.

Allelic variants of 49875, e.g., human 49875, include both functional and non-functional proteins. Functional allelic variants are naturally occurring amino acid sequence variants of the 49875 protein within a population that maintain the ability to bind an NTP, e.g., ATP, and/or unwind a nucleic acid duplex. Functional allelic variants will typically contain only conservative substitution of one or more amino acids of SEQ ID NO:8, or substitution, deletion or insertion of non-critical residues in non-critical regions of the protein. Non-functional allelic variants are naturally-occurring amino acid sequence variants of the 49875, e.g., human 49875, protein within a population that do not have the ability to bind an NTP, e.g., ATP, and/or unwind a nucleic acid duplex. Non-functional allelic variants will typically contain a non-conservative substitution, a deletion, or insertion, or premature truncation of the amino acid sequence of SEQ ID NO:8, or a substitution, insertion, or deletion in critical residues or critical regions of the protein.

Preferred 46842 variants include those that are correlated with Arf GTPase stimulating activity.

Allelic variants of 46842, e.g., human 46842, include both functional and non-functional proteins. Functional allelic variants are naturally occurring amino acid sequence variants of the 46842 protein within a population that maintain the ability to interact with, e.g., bind to, Arf proteins and phosphoinositides. Functional allelic variants will typically contain only conservative substitution of one or more amino acids of SEQ ID NO:11, or substitution, deletion or insertion of non-critical residues in non-critical regions of the protein. Non-functional allelic variants are naturally-occurring amino acid sequence variants of the 46842, e.g., human 46842, protein within a population that do not have the ability to interact with, e.g., bind to, Arf proteins and phosphoinositides. Non-functional allelic variants will typically contain a non-conservative substitution, a deletion, or insertion, or premature truncation of the amino acid sequence of SEQ ID NO:11, or a substitution, insertion, or deletion in critical residues or critical regions of the protein.

Preferred 33201 variants include those that are correlated with the ability to metabolize alcohols and/or the ability to catalyze the reduction of quinines.

Allelic variants of 33201, e.g., human 33201, include both functional and non-functional proteins. Functional allelic variants are naturally occurring amino acid sequence variants of the 33201 protein within a population that maintain the ability to metabolize alcohols and/or the ability to catalyze the reduction of quinones. Functional allelic variants will typically contain only conservative substitution of one or more amino acids of SEQ ID NO:14, or substitution, deletion or insertion of non-critical residues in non-critical regions of the protein. Non-functional allelic variants are naturally-occurring amino acid sequence variants of the 33201, e.g., human 33201, protein within a population that do not have the ability to metabolize alcohols and/or the ability to catalyze the reduction of quinones. Non-functional allelic variants will typically contain a non-conservative substitution, a deletion, or insertion, or premature truncation of the amino acid sequence of SEQ ID NO:14, or a substitution, insertion, or deletion in critical residues or critical regions of the protein.

Preferred 83378, 84233, 64708, 85041, or 84234 variants include those that are correlated with the ability to facilitate cation diffusion.

Allelic variants of 83378, 84233, 64708, 85041, or 84234, e.g., human 83378, 84233, 64708, 85041, or 84234, include both functional and non-functional proteins. Functional allelic variants are naturally occurring amino acid sequence variants of the 83378, 84233, 64708, 85041, or 84234 protein within a population that maintain the ability to facilitate cation diffusion. Functional allelic variants will typically contain only conservative substitution of one or more amino acids of SEQ ID NO:17, SEQ ID NO:20, SEQ ID NO:23, SEQ ID NO:26, or SEQ ID NO:29, or substitution, deletion or insertion of non-critical residues in non-critical regions of the protein. Non-functional allelic variants are naturally-occurring amino acid sequence variants of the 83378, 84233, 64708, 85041, or 84234, e.g., human 83378, 84233, 64708, 85041, or 84234, protein within a population that do not have the ability to facilitate cation diffusion. Non-functional allelic variants will typically contain a non-conservative substitution, a deletion, or insertion, or premature truncation of the amino acid sequence of SEQ ID NO:17, SEQ ID NO:20, SEQ ID NO:23, SEQ ID NO:26, or SEQ ID NO:29, or a substitution, insertion, or deletion in critical residues or critical regions of the protein.

Moreover, nucleic acid molecules encoding other 47476, 67210, 49875, 46842, 33201, 83378, 84233, 64708, 85041, or 84234 family members and, thus, which have a nucleotide sequence which differs from the 47476, 67210, 49875, 46842, 33201, 83378, 84233, 64708, 85041, or 84234 sequences of SEQ ID NO:1, SEQ ID NO:4, SEQ ID NO:7, SEQ ID NO:10, SEQ ID NO:13, SEQ ID NO:16, SEQ ID NO:19, SEQ ID NO:22, SEQ ID NO:25, or SEQ ID NO:28, or SEQ ID NO:3, SEQ ID NO:6, SEQ ID NO:9, SEQ ID NO:12, SEQ ID NO:15, SEQ ID NO:18, SEQ ID NO:21, SEQ ID NO:24, SEQ ID NO:27, or SEQ ID NO:30 are intended to be within the scope of the invention.

Antisense Nucleic Acid Molecules, Ribozymes, and Modified 47476, 67210, 49875, 46842, 33201, 83378, 84233, 64708, 85041, or 84234 Nucleic Acid Molecules

In another aspect, the invention features, an isolated nucleic acid molecule which is antisense to 47476, 67210, 49875, 46842, 33201, 83378, 84233, 64708, 85041, or 84234. An “antisense” nucleic acid can include a nucleotide sequence which is complementary to a “sense” nucleic acid encoding a protein, e.g., complementary to the coding strand of a double-stranded cDNA molecule or complementary to an mRNA sequence. The antisense nucleic acid can be complementary to an entire 47476, 67210, 49875, 46842, 33201, 83378, 84233, 64708, 85041, or 84234 coding strand, or to only a portion thereof (e.g., the coding region of human 47476, 67210, 49875, 46842, 33201, 83378, 84233, 64708, 85041, or 84234 corresponding to SEQ ID NO:1, SEQ ID NO:4, SEQ ID NO:7, SEQ ID NO:10, SEQ ID NO:13, SEQ ID NO:16, SEQ ID NO:19, SEQ ID NO:22, SEQ ID NO:25, or SEQ ID NO:28, or SEQ ID NO:3, SEQ ID NO:6, SEQ ID NO:9, SEQ ID NO:12, SEQ ID NO:15, SEQ ID NO:18, SEQ ID NO:21, SEQ ID NO:24, SEQ ID NO:27, or SEQ ID NO:30. In another embodiment, the antisense nucleic acid molecule is antisense to a “noncoding region” of the coding strand of a nucleotide sequence encoding 47476, 67210, 49875, 46842, 33201, 83378, 84233, 64708, 85041, or 84234 (e.g., the 5′ and 3′ untranslated regions).

An antisense nucleic acid can be designed such that it is complementary to the entire coding region of 47476, 67210, 49875, 46842, 33201, 83378, 84233, 64708, 85041, or 84234 mRNA, but more preferably is an oligonucleotide which is antisense to only a portion of the coding or noncoding region of 47476, 67210, 49875, 46842, 33201, 83378, 84233, 64708, 85041, or 84234 mRNA. For example, the antisense oligonucleotide can be complementary to the region surrounding the translation start site of 47476, 67210, 49875, 46842, 33201, 83378, 84233, 64708, 85041, or 84234 mRNA, e.g., between the −10 and +10 regions of the target gene nucleotide sequence of interest. An antisense oligonucleotide can be, for example, about 7, 10, 15, 20, 25, 30, 35, 40, 45, 50, 55, 60, 65, 70, 75, 80, or more nucleotides in length.

An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. The antisense nucleic acid also can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject (e.g., by direct injection at a tissue site), or generated in situ such that they hybridize with or bind to cellular mRNA and/or genomic DNA encoding a 47476, 67210, 49875, 46842, 33201, 83378, 84233, 64708, 85041, or 84234 protein to thereby inhibit expression of the protein, e.g., by inhibiting transcription and/or translation. Alternatively, antisense nucleic acid molecules can be modified to target selected cells and then administered systemically. For systemic administration, antisense molecules can be modified such that they specifically bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids. Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

In still another embodiment, an antisense nucleic acid of the invention is a ribozyme. A ribozyme having specificity for a 47476, 67210, 49875, 46842, 33201, 83378, 84233, 64708, 85041, or 84234-encoding nucleic acid can include one or more sequences complementary to the nucleotide sequence of a 47476, 67210, 49875, 46842, 33201, 83378, 84233, 64708, 85041, or 84234 cDNA disclosed herein (i.e., SEQ ID NO:1, SEQ ID NO:4, SEQ ID NO:7, SEQ ID NO:10, SEQ ID NO:13, SEQ ID NO:16, SEQ ID NO:19, SEQ ID NO:22, SEQ ID NO:25, or SEQ ID NO:28, or SEQ ID NO:3, SEQ ID NO:6, SEQ ID NO:9, SEQ ID NO:12, SEQ ID NO:15, SEQ ID NO:18, SEQ ID NO:21, SEQ ID NO:24, SEQ ID NO:27, or SEQ ID NO:30), and a sequence having known catalytic sequence responsible for mRNA cleavage (see U.S. Pat. No. 5,093,246 or Haselhoff and Gerlach (1988) Nature 334:585-591). For example, a derivative of a Tetrahymena L-19 IVS RNA can be constructed in which the nucleotide sequence of the active site is complementary to the nucleotide sequence to be cleaved in a 47476, 67210, 49875, 46842, 33201, 83378, 84233, 64708, 85041, or 84234-encoding mRNA. See, e.g., Cech et al. U.S. Pat. No. 4,987,071; and Cech et al. U.S. Pat. No. 5,116,742. Alternatively, 47476, 67210, 49875, 46842, 33201, 83378, 84233, 64708, 85041, or 84234 mRNA can be used to select a catalytic RNA having a specific ribonuclease activity from a pool of RNA molecules. See, e.g., Bartel, D. and Szostak, J. W. (1993) Science 261:1411-1418.

47476, 67210, 49875, 46842, 33201, 83378, 84233, 64708, 85041, or 84234 gene expression can be inhibited by targeting nucleotide sequences complementary to the regulatory region of the 47476, 67210, 49875, 46842, 33201, 83378, 84233, 64708, 85041, or 84234 (e.g., the 47476, 67210, 49875, 46842, 33201, 83378, 84233, 64708, 85041, or 84234 promoter and/or enhancers) to form triple helical structures that prevent transcription of the 47476, 67210, 49875, 46842, 33201, 83378, 84233, 64708, 85041, or 84234 gene in target cells. See generally, Helene, C. (1991) Anticancer Drug Des. 6:569-84; Helene, C. i (1992) Ann. N.Y. Acad. Sci. 660:27-36; and Maher, L. J. (1992) Bioassays 14:807-15. The potential sequences that can be targeted for triple helix formation can be increased by creating a so-called “switchback” nucleic acid molecule. Switchback molecules are synthesized in an alternating 5′-3′, 3′-5′ manner, such that they base pair with first one strand of a duplex and then the other, eliminating the necessity for a sizeable stretch of either purines or pyrimidines to be present on one strand of a duplex.

The invention also provides detectably labeled oligonucleotide primer and probe molecules. Typically, such labels are chemiluminescent, fluorescent, radioactive, or colorimetric.

A 47476, 67210, 49875, 46842, 33201, 83378, 84233, 64708, 85041, or 84234 nucleic acid molecule can be modified at the base moiety, sugar moiety or phosphate backbone to improve, e.g., the stability, hybridization, or solubility of the molecule. For non-limiting examples of synthetic oligonucleotides with modifications see Toulmé (2001) Nature Biotech. 19:17 and Faria et al. (2001) Nature Biotech. 19:40-44. Such phosphoramidite oligonucleotides can be effective antisense agents.

For example, the deoxyribose phosphate backbone of the nucleic acid molecules can be modified to generate peptide nucleic acids (see Hyrup B. et al. (1996) Bioorganic & Medicinal Chemistry 4: 5-23). As used herein, the terms “peptide nucleic acid” or “PNA” refers to a nucleic acid mimic, e.g., a DNA mimic, in which the deoxyribose phosphate backbone is replaced by a pseudopeptide backbone and only the four natural nucleobases are retained. The neutral backbone of a PNA can allow for specific hybridization to DNA and RNA under conditions of low ionic strength. The synthesis of PNA oligomers can be performed using standard solid phase peptide synthesis protocols as described in Hyrup B. et al. (1996) supra and Perry-O'Keefe et al. Proc. Natl. Acad. Sci. 93: 14670-675.

PNAs of 47476, 67210, 49875, 46842, 33201, 83378, 84233, 64708, 85041, or 84234 nucleic acid molecules can be used in therapeutic and diagnostic applications. For example, PNAs can be used as antisense or antigene agents for sequence-specific modulation of gene expression by, for example, inducing transcription or translation arrest or inhibiting replication. PNAs of 47476, 67210, 49875, 46842, 33201, 83378, 84233, 64708, 85041, or 84234 nucleic acid molecules can also be used in the analysis of single base pair mutations in a gene, (e.g., by PNA-directed PCR clamping); as ‘artificial restriction enzymes’ when used in combination with other enzymes, (e.g., S1 nucleases (Hyrup B. et al. (1996) supra)); or as probes or primers for DNA sequencing or hybridization (Hyrup B. et al. (1996) supra; Perry-O'Keefe supra).

In other embodiments, the oligonucleotide may include other appended groups such as peptides (e.g., for targeting host cell receptors in vivo), or agents facilitating transport across the cell membrane (see, e.g., Letsinger et al. (1989) Proc. Natl. Acad. Sci. USA 86:6553-6556; Lemaitre et al. (1987) Proc. Natl. Acad. Sci. USA 84:648-652; PCT Publication No. WO88/09810) or the blood-brain barrier (see, e.g., PCT Publication No. WO89/10134). In addition, oligonucleotides can be modified with hybridization-triggered cleavage agents (see, e.g., Krol et al. (1988) Bio-Techniques 6:958-976) or intercalating agents. (see, e.g., Zon (1988) Pharm. Res. 5:539-549). To this end, the oligonucleotide may be conjugated to another molecule, (e.g., a peptide, hybridization triggered cross-linking agent, transport agent, or hybridization-triggered cleavage agent).

The invention also includes molecular beacon oligonucleotide primer and probe molecules having at least one region which is complementary to a 47476, 67210, 49875, 46842, 33201, 83378, 84233, 64708, 85041, or 84234 nucleic acid of the invention, two complementary regions one having a fluorophore and one a quencher such that the molecular beacon is useful for quantitating the presence of the 47476, 67210, 49875, 46842, 33201, 83378, 84233, 64708, 85041, or 84234 nucleic acid of the invention in a sample. Molecular beacon nucleic acids are described, for example, in Lizardi et al., U.S. Pat. No. 5,854,033; Nazarenko et al., U.S. Pat. No. 5,866,336, and Livak et al., U.S. Pat. No. 5,876,930.

Isolated 47476 Polypeptides

In another aspect, the invention features, an isolated 47476 protein, or fragment, e.g., a biologically active portion, for use as immunogens or antigens to raise or test (or more generally to bind) anti-47476 antibodies. 47476 protein can be isolated from cells or tissue sources using standard protein purification techniques. 47476 protein or fragments thereof can be produced by recombinant DNA techniques or synthesized chemically.

Polypeptides of the invention include those that arise as a result of the existence of multiple genes, alternative transcription events, alternative RNA splicing events, and alternative translational and post-translational events. The polypeptide can be expressed in systems, e.g., cultured cells, which result in substantially the same post-translational modifications present when expressed the polypeptide is expressed in a native cell, or in systems which result in the alteration or omission of post-translational modifications, e.g., glycosylation or cleavage, present when expressed in a native cell.

In a preferred embodiment, a 47476 polypeptide has one or more of the following characteristics

(i) it has the ability to stimulate the exchange of guanine nucleotides (GTP for GDP) bound to small guanine nucleotide binding proteins, in particular, ras or ras-like proteins;

(ii) it has the ability to bind calcium;

(iii) it has the ability to bind at least one and preferably two zinc ions;

(iv) it has the ability to bind a second messenger, e.g., diacylglycerol or analogs thereof, such as phorbol esters;

(v) it has the ability to activate a ras superfamily member;

(vi) it has a molecular weight, e.g., a deduced molecular weight, preferably ignoring any contribution of post translational modifications, amino acid composition or other physical characteristic of a 47476 polypeptide, e.g., a polypeptide of SEQ ID NO:2;

(vii) it has an overall sequence similarity of at least 60%, more preferably at least 70%, 80%, 90%, 95%, 98%, 99%, or more with a polypeptide of SEQ ID NO:2;

(viii) it has a ras guanine nucleotide dissociation stimulator domain which is preferably about 70%, 80%, 90%, 95%, 98%, 99%, or more homologous to amino acid residues about 195 to 381 of SEQ ID NO:2;

(ix) it has a guanine nucleotide dissociation stimulator domain N-terminal motif which is preferably about 70%, 80%, 90%, 95%, 98%, 99%, or more homologous to amino acid residues about 55 to 172 of SEQ ID NO:2;

(x) it has an EF-hand calcium-binding domain which is preferably about 70%, 80%, 90%, 95%, 98%, 99%, or more homologous to amino acid residues about 470 to 498 of SEQ ID NO:2;

(xi) it has a phorbol ester/diacylglycerol binding domain (C1 domain) which is preferably about 70%, 80%, 90%, 95%, 98%, 99%, or more homologous to amino acid residues about 541 to 590 of SEQ ID NO:2;

(xii) it has a guanine nucleotide dissociation stimulator domain N-terminal motif which is preferably about 70%, 80%, 90%, 95%, 98%, 99%, or more homologous to amino acid residues about 55 to 172 of SEQ ID NO:2;

(xiii) it has at least one predicted N-glycosylation site (PS00001);

(xiv) it has at least one, two, three, preferably four predicted cAMP/cGMP-dependent protein kinase phosphorylation sites (PS00004);

(xv) it has at least one, two, three, four, five, six, seven, preferably eight predicted Protein Kinase C phosphorylation sites (PS00005);

(xvi) it has at least one, two, three, four, five, six, seven, eight, nine, ten, eleven, twelve, thirteen, preferably fourteen predicted Casein Kinase II phosphorylation sites (PS00006);

(xvii) it has at least one, two, three, preferably four predicted N-myristylation sites (PS00008); and

(xviii) it has at least one predicted Amidation site (PS00009).

In a preferred embodiment the 47476 protein, or fragment thereof, differs from the corresponding sequence in SEQ ID NO:2. In one embodiment it differs by at least one but by less than 15, 10 or 5 amino acid residues. In another it differs from the corresponding sequence in SEQ ID NO:2 by at least one residue but less than 20%, 15%, 10% or 5% of the residues in it differ from the corresponding sequence in SEQ ID NO:2. (If this comparison requires alignment the sequences should be aligned for maximum homology. “Looped” out sequences from deletions or insertions, or mismatches, are considered differences.) The differences are, preferably, differences or changes at a non-essential residue or a conservative substitution. In a preferred embodiment, the differences are not in the ras guanine nucleotide dissociation stimulator domain, the EF-hand calcium-binding domain or the phorbol ester/diacylglycerol binding domain (C1 domain). In another preferred embodiment, one or more differences are in the ras guanine nucleotide dissociation stimulator domain, the EF-hand calcium-binding domain, and/or the phorbol ester/diacylglycerol binding domain (C1 domain).

Other embodiments include a protein that contain one or more changes in amino acid sequence, e.g., a change in an amino acid residue which is not essential for activity. Such 47476 proteins differ in amino acid sequence from SEQ ID NO:2, yet retain biological activity.

In one embodiment, the protein includes an amino acid sequence at least about 60%, 65%, 70%, 75%, 80%, 85%, 90%, 95%, 98% or more homologous to SEQ ID NO:2.

A 47476 protein or fragment is provided which varies from the sequence of SEQ ID NO.2 in regions defined by amino acids about 1 to 54, 173 to 194, 382 to 469, 499 to 540, and 591 to 672 by at least one but by less than 15, 10 or 5 amino acid residues in the protein or fragment, but which does not differ from SEQ ID NO.2 in regions defined by amino acids about 55 to 173, 195 to 381, 470 to 498 or 541 to 590 of SEQ ID NO:2. (If this comparison requires alignment the sequences should be aligned for maximum homology. “Looped” out sequences from deletions or insertions, or mismatches, are considered differences.) In some embodiments the difference is at a non-essential residue or is a conservative substitution, while in others the difference is at an essential residue or is a non-conservative substitution.

In one embodiment, a biologically active portion of a 47476 protein includes a ras guanine nucleotide dissociation stimulator domain. Moreover, other biologically active portions, in which other regions of the protein are deleted, can be prepared by recombinant techniques and evaluated for one or more of the functional activities of a native 47476 protein.

In a preferred embodiment, the 47476 protein has an amino acid sequence shown in SEQ ID NO:2. In other embodiments, the 47476 protein is substantially identical to SEQ ID NO:2. In yet another embodiment, the 47476 protein is substantially identical to SEQ ID NO:2 and retains the functional activity of the protein of SEQ ID NO:2, as described in detail in the subsections above.

In a preferred embodiment, a 47476 polypeptide fragment differs by at least 1, 2, 3, 10, 20, or more amino acid residues of a previously disclosed sequence. Differences can include differing in length or sequence identity. For example, a fragment can: include one or more amino acid residues from SEQ ID NO:5 outside the region of overlap with the previously disclosed amino acid sequence; not include all of the amino acid residues encoded by a previously disclosed polypeptide sequence, e.g., can be one or more amino acid residues shorter (at one or both ends) than a previously disclocsed polypeptide sequence, or can differ by one or more amino acid residues in the region of overlap.

Isolated 67210 Polypeptides

In another aspect, the invention features, an isolated 67210 protein, or fragment, e.g., a biologically active portion, for use as immunogens or antigens to raise or test (or more generally to bind) anti-67210 antibodies. 67210 protein can be isolated from cells or tissue sources using standard protein purification techniques. 67210 protein or fragments thereof can be produced by recombinant DNA techniques or synthesized chemically.

Polypeptides of the invention include those which arise as a result of the existence of multiple genes, alternative transcription events, alternative RNA splicing events, and alternative translational and post-translational events. The polypeptide can be expressed in systems, e.g., cultured cells, which result in substantially the same post-translational modifications present when expressed the polypeptide is expressed in a native cell, or in systems which result in the alteration or omission of post-translational modifications, e.g., glycosylation or cleavage, present when expressed in a native cell.

In a preferred embodiment, a 67210 polypeptide has one or more of the following characteristics:

(i) it has the ability to catalyze the transfer of an activated sugar residue to an acceptor molecule;

(ii) it has the ability to catalyzes the processing, folding, and secretion of proteins;

(iii) it has a molecular weight, e.g., a deduced molecular weight, preferably ignoring any contribution of post translational modifications, amino acid composition or other physical characteristic of SEQ ID NO:5;

(iv) it has an overall sequence similarity of at least 60%, more preferably at least 70, 80, 90, or 95%, with a polypeptide of SEQ ID NO:5;

(v) it has a glycosyl transferase domain which is preferably about 70%, 80%, 90% or 95% homologous with amino acid residues about 63 to 340 of SEQ ID NO:5;

(vi) it can be found in any one or more of: coronary smooth muscle cells (SMC), normal artery, normal brain cortex, normal breast, and normal ovary;

(vii) it has at least one predicted signal peptide;

(viii) it has at least one dileucine motif;

(ix) it has at least one predicted N-glycosylation site (PS00001);

(x) it has at least one predicted Protein Kinase C phosphorylation site (PS00005);

(xi) it has at least one, two, three, preferably four predicted Casein Kinase II phosphorylation sites (PS00006);

(xii) it has at least one predicted tyrosine kinase phosphorylation site (PS00007); and

(xiii) it has at least one, two, three, four, five, preferably six predicted N-myristoylation sites (PS00008).

In a preferred embodiment the 67210 protein, or fragment thereof, differs from the corresponding sequence in SEQ ID NO:2. In one embodiment it differs by at least one but by less than 15, 10 or 5 amino acid residues. In another it differs from the corresponding sequence in SEQ ID NO:5 by at least one residue but less than 20%, 15%, 10% or 5% of the residues in it differ from the corresponding sequence in SEQ ID NO:5. (If this comparison requires alignment the sequences should be aligned for maximum homology. “Looped” out sequences from deletions or insertions, or mismatches, are considered differences.) The differences are, preferably, differences or changes at a non-essential residue or a conservative substitution. In a preferred embodiment the differences are not in the glycosyltransferase domain (amino acid 63 to 340 of SEQ ID NO:5). In another preferred embodiment one or more differences are in the glycosyltransferase domain (amino acid 63 to 340 of SEQ ID NO:5).

Other embodiments include a protein that contain one or more changes in amino acid sequence, e.g., a change in an amino acid residue which is not essential for activity. Such 67210 proteins differ in amino acid sequence from SEQ ID NO:5, yet retain biological activity.

In one embodiment, the protein includes an amino acid sequence at least about 50%, 55%, 60%, 65%, 70%, 75%, 80%, 85%, 90%, 95%, 98% or more homologous to SEQ ID NO:5.

A 67210 protein or fragment is provided which varies from the sequence of SEQ ID NO:5 in regions defined by amino acids about 1 to 62 and 341 to 349 by at least one but by less than 15, 10 or 5 amino acid residues in the protein or fragment but which does not differ from SEQ ID NO:5 in regions defined by amino acids about 63 to 340. (If this comparison requires alignment the sequences should be aligned for maximum homology. “Looped” out sequences from deletions or insertions, or mismatches, are considered differences.) In some embodiments the difference is at a non-essential residue or is a conservative substitution, while in others the difference is at an essential residue or is a non-conservative substitution.

In one embodiment, a biologically active portion of a 67210 protein includes a glycosyltransferase domain. Moreover, other biologically active portions, in which other regions of the protein are deleted, can be prepared by recombinant techniques and evaluated for one or more of the functional activities of a native 67210 protein.

In a preferred embodiment, the 67210 protein has an amino acid sequence shown in SEQ ID NO:5. In other embodiments, the 67210 protein is substantially identical to SEQ ID NO:5. In yet another embodiment, the 67210 protein is substantially identical to SEQ ID NO:5 and retains the functional activity of the protein of SEQ ID NO:5, as described in detail in the subsections above.

In a preferred embodiment, a fragment differs by at least 1, 2, 3, 10, 20, or more amino acid residues encoded by a sequence present in Genbank accession number AC013776 or AC023550. Differences can include differing in length or sequence identity. For example, a fragment can: include one or more amino acid residues from SEQ ID NO:5 outside the region encoded by nucleotides 290 to 560, 675 to 1042, or 1152 to 1743 of SEQ ID NO:4; not include all of the amino acid residues encoded by a nucleotide sequence in Genbank accession number AC013776 or AC023550, e.g., can be one or more amino acid residues shorter (at one or both ends) than a sequence encoded by the nucleotide sequence in Genbank accession number AC013776 or AC023550, or can differ by one or more amino acid residues in the region of overlap.

Isolated 49875 Polypeptides

In another aspect, the invention features, an isolated 49875 protein, or fragment, e.g., a biologically active portion, for use as immunogens or antigens to raise or test (or more generally to bind) anti-49875 antibodies. 49875 protein can be isolated from cells or tissue sources using standard protein purification techniques. 49875 protein or fragments thereof can be produced by recombinant DNA techniques or synthesized chemically.

Polypeptides of the invention include those that arise as a result of the existence of multiple genes, alternative transcription events, alternative RNA splicing events, and alternative translational and post-translational events. The polypeptide can be expressed in systems, e.g., cultured cells, which result in substantially the same post-translational modifications present when expressed the polypeptide is expressed in a native cell, or in systems which result in the alteration or omission of post-translational modifications, e.g., glycosylation or cleavage, present when expressed in a native cell.

In a preferred embodiment, a 49875 polypeptide has one or more of the following characteristics:

(i) it has the ability to bind and hydrolyze ATG or GTP;

(ii) it has the ability to break the hydrogen bonds between the two strands of a nucleic acid duplex and unwind the duplex;

(iii) it has the ability to modulate replication;

(iv) it has the ability to modulate transcription or translation;

(v) it has a molecular weight, e.g., a deduced molecular weight, preferably ignoring any contribution of post translational modifications, amino acid composition or other physical characteristic of SEQ ID NO:8;

(vi) it has an overall sequence similarity of at least 60%, more preferably at least 70%, 80%, 90%, 95%, 98%, 99%, or more with a polypeptide of SEQ ID NO:8;

(vii) it has a DEAD-type helicase domain which is preferably about 70%, 80%, 90%, 95%, 98%, 99%, or more homologous with amino acid residues about 22 to 245 of SEQ ID NO:8; or

(vii) it has a conserved helicase C-terminal domain which is preferably about 70%, 80%, 90%, 95%, 98%, 99%, or more homologous with amino acid residues about 281 to 363 of SEQ ID NO:8.

(viii) it has a conserved DEAD-box subfamily ATP-dependent helicase signature motif (PS00039);

(ix) it can be found in, e.g., breast, colon, lung and/or ovary normal and tumor tissue and placenta.

(x) it has at least one, preferably two predicted N-glycosylation sites;

(xi) it has at least one, two, three, four, preferably five predicted Protein Kinase C phosphorylation sites (PS00005);

(xii) it has at least one, two, three, four, five, six, preferably seven predicted Casein Kinase II phosphorylation sites (PS00006);

(xiii) it has at least one, two, three, four, five, six, preferably seven predicted N-myristoylation sites (PS00008);

(xiv) it has at least one, preferably two predicted amidation sites (PS00009); or

(xv) it has at least one predicted ATP/GTP-binding site motif A (P-loop) (PS00017).

In a preferred embodiment the 49875 protein, or fragment thereof, differs from the corresponding sequence in SEQ ID:8. In one embodiment it differs by at least one but by less than 15, 10 or 5 amino acid residues. In another it differs from the corresponding sequence in SEQ ID NO:8 by at least one residue but less than 20%, 15%, 10% or 5% of the residues in it differ from the corresponding sequence in SEQ ID NO:8. (If this comparison requires alignment the sequences should be aligned for maximum homology. “Looped” out sequences from deletions or insertions, or mismatches, are considered differences.) The differences are, preferably, differences or changes at a non essential residue or a conservative substitution. In a preferred embodiment the differences are not in the DEAD type helicase domain (amino acids 22-245 of SEQ ID NO:8). In another preferred embodiment one or more differences are in the DEAD type helicase domain (amino acids 22-245 of SEQ ID NO:8).

Other embodiments include a protein that contain one or more changes in amino acid sequence, e.g., a change in an amino acid residue which is not essential for activity. Such 49875 proteins differ in amino acid sequence from SEQ ID NO:8, yet retain biological activity.

In one embodiment, the protein includes an amino acid sequence at least about 60%, 65%, 70%, 75%, 80%, 85%, 90%, 95%, 98% or more homologous to SEQ ID NO:8.

A 49875 protein or fragment is provided which varies from the sequence of SEQ ID NO:8 in regions defined by amino acids about amino acids 1-21 and 246-600 of SEQ ID NO:8 by at least one but by less than 15, 10 or 5 amino acid residues in the protein or fragment but which does not differ from SEQ ID NO:8 in regions defined by amino acids about 22-245. (If this comparison requires alignment the sequences should be aligned for maximum homology. “Looped” out sequences from deletions or insertions, or mismatches, are considered differences.) In some embodiments the difference is at a non-essential residue or is a conservative substitution, while in others the difference is at an essential residue or is a non-conservative substitution.

In one embodiment, a biologically active portion of a 49875 protein includes a DEAD type helicase domain or a conserved C-terminal helicase domain. Moreover, other biologically active portions, in which other regions of the protein are deleted, can be prepared by recombinant techniques and evaluated for one or more of the functional activities of a native 49875 protein.

In a preferred embodiment, the 49875 protein has an amino acid sequence shown in SEQ ID NO:8. In other embodiments, the 49875 protein is substantially identical to SEQ ID NO:8. In yet another embodiment, the 49875 protein is substantially identical to SEQ ID NO:8 and retains the functional activity of the protein of SEQ ID NO:8, as described in detail in the subsections above.

In a preferred embodiment, a fragment differs by at least 1, 2, 3, 10, 20, or more amino acid residues encoded by a sequence present in WO 01/55301, WO 01/57188 or WO 01/62927. Differences can include differing in length or sequence identity. For example, a fragment can: not include all of the amino acid residues encoded by a nucleotide sequence in WO 01/55301, WO 01/57188 or WO 01/62927, e.g., can be one or more amino acid residues shorter (at one or both ends) than a sequence encoded by a nucleotide sequence in WO 01/55301, WO 01/57188 or WO 01/62927 or can differ by one or more amino acid residues in the region of overlap.

Isolated 46842 Polypeptides

In another aspect, the invention features, an isolated 46842 protein, or fragment, e.g., a biologically active portion, for use as immunogens or antigens to raise or test (or more generally to bind) anti-46842 antibodies. 46842 protein can be isolated from cells or tissue sources using standard protein purification techniques. 46842 protein or fragments thereof can be produced by recombinant DNA techniques or synthesized chemically.

Polypeptides of the invention include those which arise as a result of the existence of multiple genes, alternative transcription events, alternative RNA splicing events, and alternative translational and post-translational events. The polypeptide can be expressed in systems, e.g., cultured cells, which result in substantially the same post-translational modifications present when expressed the polypeptide is expressed in a native cell, or in systems which result in the alteration or omission of post-translational modifications, e.g., glycosylation or cleavage, present when expressed in a native cell.

In a preferred embodiment, A 46842 polypeptide has one or more of the following characteristics:

(i) it has the ability to stimulate GTP hydrolysis of GTP bound by an Arf or Arf-like protein;

(ii) it has the ability to respond to phosphoinositide second messengers

(iii) it has a molecular weight, e.g., a deduced molecular weight, preferably ignoring any contribution of post translational modifications, amino acid composition or other physical characteristic of SEQ ID NO:11;

(iv) it has an overall sequence similarity of at least 60%, more preferably at least 70%, 80%, 90%, 95%, 98%, 99%, or more with a polypeptide of SEQ ID NO:11;

(v) it has a PH domain which is preferably about 70%, 80%, 90%, 95%, 98%, 99%, or more homologous with amino acid residues about 269 to 363 of SEQ ID NO:11;

(vi) it has an ArfGAP domain which is preferably about 70%, 80%, 90%, 95%, 98%, 99%, or more homologous with amino acid residues about 403 to 525 of SEQ ID NO:11;

(vii) it has at least one, preferably two ankyrin domains which are preferably about 70%, 80%, 90%, 95%, 98%, 99%, or more homologous with amino acid residues about 702 to 734 or 735 to 767 of SEQ ID NO:11;

(viii) it can colocalize with Arf proteins;

(ix) it can localize to a vesicle membrane and/or the plasma membrane;

(x) it has the conserved cysteines for coordinating a zinc ion at about residues 421, 418, 438, AND 440° F. SEQ ID NO:1;

(xi) it has at least one, two, three, four, five, six, seven, eight, nine, ten, eleven, preferably twelve predicted Protein Kinase C phosphorylation sites (PS00005);

(xii) it has at least one, two, three, four, five, six, seven, eight, nine, ten, eleven, twelve, thirteen, preferably fourteen predicted Casein Kinase II phosphorylation sites (PS00006);

(xiii) it has at least one, two, preferably three predicted cAMP/cGMP-dependent protein kinase phosphorylation sites (PS00004);

(xiv) it has at least one predicted tyrosine kinase phosphorylation site (PS00007);

(xv) it has at least one predicted glycosaminoglycan attachment site (PS00002); and

(xvi) it has at least one, two, three, four, five, six, seven, eight, nine, preferably ten predicted N-myristylation sites (PS00008).

In a preferred embodiment the 46842 protein, or fragment thereof, differs from the corresponding sequence in SEQ ID NO:11. In one embodiment it differs by at least one but by less than 15, 10 or 5 amino acid residues. In another it differs from the corresponding sequence in SEQ ID NO:11 by at least one residue but less than 20%, 15%, 10% or 5% of the residues in it differ from the corresponding sequence in SEQ ID NO:11. (If this comparison requires alignment the sequences should be aligned for maximum homology. “Looped” out sequences from deletions or insertions, or mismatches, are considered differences.) The differences are, preferably, differences or changes at a non essential residue or a conservative substitution. In a preferred embodiment the differences are not in the ArfGAP, PH or ankyrin repeat domains. In another preferred embodiment one or more differences are in the ArfGAP, PH or ankyrin repeat domains.

Other embodiments include a protein that contain one or more changes in amino acid sequence, e.g., a change in an amino acid residue which is not essential for activity. Such 46842 proteins differ in amino acid sequence from SEQ ID NO:11, yet retain biological activity.

In one embodiment, the protein includes an amino acid sequence at least about 50%, 55%, 60%, 65%, 70%, 75%, 80%, 85%, 90%, 95%, 98% or more homologous to SEQ ID NO:11.

A 46842 protein or fragment is provided which varies from the sequence of SEQ ID NO:11 in regions defined by amino acids about 403 to 525 by at least one but by less than 15, 10 or 5 amino acid residues in the protein or fragment but which does not differ from SEQ ID NO:11 in regions defined by amino acids about 1 to 402, and/or 526 to 834. (If this comparison requires alignment the sequences should be aligned for maximum homology. “Looped” out sequences from deletions or insertions, or mismatches, are considered differences.) In some embodiments the difference is at a non-essential residue or is a conservative substitution, while in others the difference is at an essential residue or is a non-conservative substitution.

In one embodiment, a biologically active portion of a 46842 protein includes an ArfGAP domain, a PH domain, or an ankyrin repeat domain. Moreover, other biologically active portions, in which other regions of the protein are deleted, can be prepared by recombinant techniques and evaluated for one or more of the functional activities of a native 46842 protein.

In a preferred embodiment, the 46842 protein has an amino acid sequence shown in SEQ ID NO:11. In other embodiments, the 46842 protein is substantially identical to SEQ ID NO:11. In yet another embodiment, the 46842 protein is substantially identical to SEQ ID NO:11 and retains the functional activity of the protein of SEQ ID NO:11, as described in detail in the subsections above. In still another embodiment, the protein includes the motif SLSSDSGLG at about amino acids 606 to 614 of SEQ ID NO:11.

In a preferred embodiment, a fragment differs by at least 1, 2, 3, 10, 20, or more amino acid residues encoded by a sequence present in KIAA1716 (see, e.g., Genbank accession number gi12697977). Differences can include differing in length or sequence identity. For example, a fragment can: include one or more amino acid residues from SEQ ID NO:11 outside the region of about amino acid residue 200 to 500, 300 to 600, or 400 to 834; not include all of the amino acid residues of the sequence in Genbank accession number gi12697977, e.g., can be one or more amino acid residues shorter (at one or both ends) than a sequence encoded by the nucleotide sequence in Genbank accession number gi12697977, or can differ by one or more amino acid residues in the region of overlap.

Isolated 33201 Polypeptides

In another aspect, the invention features, an isolated 33201 protein, or fragment, e.g., a biologically active portion, for use as immunogens or antigens to raise or test (or more generally to bind) anti-33201 antibodies. 33201 protein can be isolated from cells or tissue sources using standard protein purification techniques. 33201 protein or fragments thereof can be produced by recombinant DNA techniques or synthesized chemically.

Polypeptides of the invention include those which arise as a result of the existence of multiple genes, alternative transcription events, alternative RNA splicing events, and alternative translational and post-translational events. The polypeptide can be expressed in systems, e.g., cultured cells, which result in substantially the same post-translational modifications present when expressed the polypeptide is expressed in a native cell, or in systems which result in the alteration or omission of post-translational modifications, e.g., glycosylation or cleavage, present when expressed in a native cell.

In a preferred embodiment, a 33201 polypeptide has one or more of the following characteristics:

(i) it has the ability to metabolize one or more alcohols;

(ii) it has the ability to catalyze the reduction of quinones;

(iii) it has the ability to reduce a bioreductive compound, e.g., a bioreductive antitumor quinone;

(iv) it has the abilitity to metabolize and/or degrade toxins;

(v) it has a molecular weight, e.g., a deduced molecular weight, preferably ignoring any contribution of post-translational modifications, amino acid composition or other physical characteristic of a 33201 polypeptide, e.g., a polypeptide pf a SEQ ID NO:14;

(vi) it has an overall sequence similarity of at least 60%, more preferably at least 70%, 80%, 90%, 95%, 98%, 99%, or more with a polypeptide of SEQ ID NO:14;

(vii) it has a dehydrogenase/reductase domain which is preferably about 70%, 80%, 90%, 95%, 98%, 99%, or more homolgous with amino acid residues about 22 to 345 of SEQ ID NO:14;

(viii) it has at least two, preferably four, five, six and most preferably seven of the cysteines found in the amino acid sequence of the native protein;

(ix) it has at least one, two, preferably three conserved glycine residues;

(x) it has at least one predicted Protein Kinase C phosphorylation site (PS00005);

(xi) it has at least one, preferably two predicted Casein Kinase II phosphorylation sites (PS00006);

(xii) it has at least one, two, three, preferably four predicted N-glycosylation sites (PS00001); and

(xiii) it has at least one, two, three, four, five, six, seven, preferably eight predicted N-myristylation sites (PS00008).

In a preferred embodiment the 33201 protein, or fragment thereof, differs from the corresponding sequence in SEQ ID NO:14. In one embodiment it differs by at least one but by less than 15, 10 or 5 amino acid residues. In another it differs from the corresponding sequence in SEQ ID NO:14 by at least one residue but less than 20%, 15%, 10% or 5% of the residues in it differ from the corresponding sequence in SEQ ID NO:14. (If this comparison requires alignment the sequences should be aligned for maximum homology. “Looped” out sequences from deletions or insertions, or mismatches, are considered differences.) The differences are, preferably, differences or changes at a non-essential residue or a conservative substitution. In a preferred embodiment the differences are not in the dehydrogenase/reductase domain (e.g., about amino acids 55-380 of SEQ ID NO:14). In another preferred embodiment one or more differences are in the dehydrogenase/reductase domain. (e.g., about amino acids 55-380 of SEQ ID NO:14).

Other embodiments include a protein that contain one or more changes in amino acid sequence, e.g., a change in an amino acid residue which is not essential for activity. Such 33201 proteins differ in amino acid sequence from SEQ ID NO:14, yet retain biological activity.

In one embodiment, the protein includes an amino acid sequence at least about 50%, 55%, 60%, 65%, 70%, 75%, 80%, 85%, 90%, 95%, 98% or more homologous to SEQ ID NO:14.

A 33201 protein or fragment is provided which varies from the sequence of SEQ ID NO:14 in regions defined by amino acids about 1 to 21 and/or 346 to 351 of SEQ ID NO:14 by at least one but by less than 15, 10 or 5 amino acid residues in the protein or fragment, but which does not differ from SEQ ID NO:14 in regions defined by amino acids about 22 to 345 of SEQ ID NO:14. (If this comparison requires alignment the sequences should be aligned for maximum homology. “Looped” out sequences from deletions or insertions, or mismatches, are considered differences.) In some embodiments the difference is at a non-essential residue or is a conservative substitution, while in others the difference is at an essential residue or is a non-conservative substitution.

In one embodiment, a biologically active portion of a 33201 protein includes a dehydrogenase/reductase domain. Moreover, other biologically active portions, in which other regions of the protein are deleted, can be prepared by recombinant techniques and evaluated for one or more of the functional activities of a native 33201 protein.

In a preferred embodiment, the 33201 protein has an amino acid sequence shown in SEQ ID NO:14. In other embodiments, the 33201 protein is substantially identical to SEQ ID NO:14. In yet another embodiment, the 33201 protein is substantially identical to SEQ ID NO:14 and retains the functional activity of the protein of SEQ ID NO:14, as described in detail in the subsections above.

In a preferred embodiment, a fragment differs by at least 1, 2, 3, 10, 20, or more amino acid residues encoded by a sequence present in Genbank accession number AC005520. Differences can include differing in length or sequence identity. For example, a fragment can include one or more amino acid residues from SEQ ID NO:14 outside the region encoded by nucleotides 1117 to 1423, 1425 to 1627, or 1629 to 1717; not include all of the amino acid residues encoded by a nucleotide sequence in Genbank accession number AC005520, e.g., can be one or more amino acid residues shorter (at one or both ends) than a sequence encoded by the nucleotide sequence in Genbank accession number AC005520; or can differ by one or more amino acid residues in the region of overlap.

Isolated 83378, 84233, 64708, 85041, or 84234 Polypeptides

In another aspect, the invention features, an isolated 83378, 84233, 64708, 85041, or 84234 protein, or fragment, e.g., a biologically active portion, for use as immunogens or antigens to raise or test (or more generally to bind) anti-83378, 84233, 64708, 85041, or 84234 antibodies. 83378, 84233, 64708, 85041, or 84234 protein can be isolated from cells or tissue sources using standard protein purification techniques. 83378, 84233, 64708, 85041, or 84234 protein or fragments thereof can be produced by recombinant DNA techniques or synthesized chemically.

Polypeptides of the invention include those which arise as a result of the existence of multiple genes, alternative transcription events, alternative RNA splicing events, and alternative translational and post-translational events. The polypeptide can be expressed in systems, e.g., cultured cells, which result in substantially the same post-translational modifications present when expressed the polypeptide is expressed in a native cell, or in systems which result in the alteration or omission of post-translational modifications, e.g., glycosylation or cleavage, present when expressed in a native cell.

In a preferred embodiment, a 83378, 84233, 64708, 85041, or 84234 polypeptide has one or more of the following characteristics:

(i) it has the ability to modulate cellular tolerance and/or resistance to a metal ion, e.g., zinc;

(ii) it has the ability to facilitate cation diffusion;

(iii) it has the ability to modulate cellular efflux of a metal ion, e.g., zinc;

(iv) it has the ability to modulate vesicular sequestration of a metal ion, e.g., zinc;

(v) it has a molecular weight, e.g., a deduced molecular weight, preferably ignoring any contribution of post translational modifications, amino acid composition or other physical characteristic of SEQ ID NO:17, SEQ ID NO:20, SEQ ID NO:23, SEQ ID NO:26, or SEQ ID NO:29;

(vi) it has an overall sequence similarity of at least 60%, more preferably at least 70%, 80%, 90%, 95%, 98%, 99%, or more with a polypeptide of SEQ ID NO:17, SEQ ID NO:20, SEQ ID NO:23, SEQ ID NO:26, or SEQ ID NO:29;

(vii) it can be found in a membrane of a cell, e.g., a plasma membrane or a vesicular membrane;

(viii) it has a cation efflux domain which a sequence similarity of preferably about 70%, 80%, 90% or 95% with about amino acid residues 11 to 133 or 231 to 389 of SEQ ID NO:17, residues 25 to 310 of SEQ ID NO:20, residues 55 to 153 or 227 to 320 of SEQ ID NO:23, residues 419 to 733 of SEQ ID NO:26, or residues 38 to 349 of SEQ ID NO:29; or

(ix) it has at least one, two, three, four, five, six, preferably seven, or more histidine residues located on polypeptide regions present in the cytoplasm.

In a preferred embodiment the 83378, 84233, 64708, 85041, or 84234 protein, or fragment thereof, differs from the corresponding sequence in SEQ ID NO:17, SEQ ID NO:20, SEQ ID NO:23, SEQ ID NO:26, or SEQ ID NO:29. In one embodiment it differs by at least one but by less than 15, 10 or 5 amino acid residues. In another it differs from the corresponding sequence in SEQ ID NO:17, SEQ ID NO:20, SEQ ID NO:23, SEQ ID NO:26, or SEQ ID NO:29 by at least one residue but less than 20%, 15%, 10% or 5% of the residues in it differ from the corresponding sequence in SEQ ID NO:17, SEQ ID NO:20, SEQ ID NO:23, SEQ ID NO:26, or SEQ ID NO:29. (If this comparison requires alignment the sequences should be aligned for maximum homology. “Looped” out sequences from deletions or insertions, or mismatches, are considered differences.) The differences are, preferably, differences or changes at a non essential residue or a conservative substitution. In a preferred embodiment the differences are not in the cation efflux domain. In another preferred embodiment one or more differences are in the cation efflux domain.

Other embodiments include a protein that contain one or more changes in amino acid sequence, e.g., a change in an amino acid residue which is not essential for activity. Such 83378, 84233, 64708, 85041, or 84234 proteins differ in amino acid sequence from SEQ ID NO:17, SEQ ID NO:20, SEQ ID NO:23, SEQ ID NO:26, or SEQ ID NO:29, yet retain biological activity.

In one embodiment, the protein includes an amino acid sequence at least about 50%, 55%, 60%, 65%, 70%, 75%, 80%, 85%, 90%, 95%, 98% or more homologous to SEQ ID NO:17, SEQ ID NO:20, SEQ ID NO:23, SEQ ID NO:26, or SEQ ID NO:29.

A 83378 protein or fragment is provided which varies from the sequence of SEQ ID NO:17 in regions defined by amino acids about 1-10, 134-230, or 390-485 by at least one but by less than 15, 10 or 5 amino acid residues in the protein or fragment but which does not differ from SEQ ID NO:17 in regions defined by amino acids about 11-133 or 231-389. A 84233 protein or fragment is provided which varies from the sequence of SEQ ID NO:20 in regions defined by amino acids about 1-24 or 311-320 by at least one but by less than 15, 10 or 5 amino acid residues in the protein or fragment but which does not differ from SEQ ID NO:20 in regions defined by amino acids about 25-310. A64708 protein or fragment is provided which varies from the sequence of SEQ ID NO:23 in regions defined by amino acids about 1-54, 154-226, or 321-461 by at least one but by less than 15, 10 or 5 amino acid residues in the protein or fragment but which does not differ from SEQ ID NO:23 in regions defined by amino acids about 55-153 or 227-320. A 85041 protein or fragment is provided which varies from the sequence of SEQ ID NO:26 in regions defined by amino acids about 1-418 or 734-765 by at least one but by less than 15, 10 or 5 amino acid residues in the protein or fragment but which does not differ from SEQ ID NO:26 in regions defined by amino acids about 419-733. A 84234 protein or fragment is provided which varies from the sequence of SEQ ID NO:29 in regions defined by amino acids about 1-37 or 350-376 by at least one but by less than 15, 10 or 5 amino acid residues in the protein or fragment but which does not differ from SEQ ID NO:29 in regions defined by amino acids about 38-349. (If this comparison requires alignment the sequences should be aligned for maximum homology. “Looped” out sequences from deletions or insertions, or mismatches, are considered differences.) In some embodiments the difference is at a non-essential residue or is a conservative substitution, while in others the difference is at an essential residue or is a non-conservative substitution.

In one embodiment, a biologically active portion of a 83378, 84233, 64708, 85041, or 84234 protein includes a cation efflux domain. Moreover, other biologically active portions, in which other regions of the protein are deleted, can be prepared by recombinant techniques and evaluated for one or more of the functional activities of a native 83378, 84233, 64708, 85041, or 84234 protein.

In a preferred embodiment, the 83378, 84233, 64708, 85041, or 84234 protein has an amino acid sequence shown in SEQ ID NO:17, SEQ ID NO:20, SEQ ID NO:23, SEQ ID NO:26, or SEQ ID NO:29. In other embodiments, the 83378, 84233, 64708, 85041, or 84234 protein is substantially identical to SEQ ID NO:17, SEQ ID NO:20, SEQ ID NO:23, SEQ ID NO:26, or SEQ ID NO:29. In yet another embodiment, the 83378,84233,64708, 85041, or 84234 protein is substantially identical to SEQ ID NO:17, SEQ ID NO:20, SEQ ID NO:23, SEQ ID NO:26, or SEQ ID NO:29 and retains the functional activity of the protein of SEQ ID NO:17, SEQ ID NO:20, SEQ ID NO:23, SEQ ID NO:26, or SEQ ID NO:29, as described in detail in the subsections above.

In a preferred embodiment, a 83378 fragment differs by at least 1, 2, 3, 10, 20, or more amino acid residues encoded by a sequence present in Genbank accession number AL359609 or a sequence disclosed in WO 01/62918, WO 01/55314, WO 01/55355. Differences can include differing in length or sequence identity. For example, a fragment can: include one or more amino acid residues from SEQ ID NO:17 outside the region encoded by nucleotides 7-1044, 1085-1638, 1253-1638, 1299-1638, 723-1775 of SEQ ID NO:16; not include all of the amino acid residues encoded by a nucleotide sequence in Genbank accession number AL359609 or a sequence disclosed in WO 01/62918, WO 01/55314, WO 01/55355, e.g., can be one or more amino acid residues shorter (at one or both ends) than a sequence encoded by the nucleotide sequence in Genbank accession number AL359609 or a sequence disclosed in WO 01/62918, WO 01/55314, WO 01/55355; or can differ by one or more amino acid residues in the region of overlap.

In a preferred embodiment, a 84233 fragment differs by at least 1, 2, 3, 10, 20, or more amino acid residues encoded by a sequence present in Genbank accession number AX061210 or AX086187 or a sequence disclosed in WO 01/12659, WO 01/51628, or WO 00/78953. Differences can include differing in length or sequence identity. For example, a fragment can: include one or more amino acid residues from SEQ ID NO:20 outside the region encoded by nucleotides 420-2103, 998-1589, 1-429, 1146-1582, 996-1425, 998-1341, 996-1379, 996-1368 of SEQ ID NO:19; not include all of the amino acid residues encoded by a nucleotide sequence in Genbank accession number AX061210 or AX086187 or a sequence disclosed in WO 01/12659, WO 01/51628, or WO 00/78953, e.g., can be one or more amino acid residues shorter (at one or both ends) than a sequence encoded by the nucleotide sequence in Genbank accession number AX061210 or AX086187 or a sequence disclosed in WO 01/12659, WO 01/51628, or WO 00/78953; or can differ by one or more amino acid residues in the region of overlap.

In a preferred embodiment, a 64708 fragment differs by at least 1, 2, 3, 10, 20, or more amino acid residues encoded by a sequence present in Genbank accession number AK000844 or a sequence disclosed in WO 01/57188, WO 01/57270, WO 01/57272, WO 01/57275, WO 01/57276, WO 01/57277, or WO 01/57278. Differences can include differing in length or sequence identity. For example, a fragment can: include one or more amino acid residues from SEQ ID NO:23 outside the region encoded by nucleotides 209-697, 918-1456, 918-1418, 1348-1790, 20-442, 911-1280, or 911-1226 of SEQ ID NO:22; not include all of the amino acid residues encoded by a nucleotide sequence in Genbank accession number AK000844 or a sequence disclosed in WO 01/57188, WO 01/57270, WO 01/57272, WO 01/57275, WO 01/57276, WO 01/57277, or WO 01/57278, e.g., can be one or more amino acid residues shorter (at one or both ends) than a sequence encoded by the nucleotide sequence in Genbank accession number AK000844 or a sequence disclosed in WO 01/57188, WO 01/57270, WO 01/57272, WO 01/57275, WO 01/57276, WO 01/57277, or WO 01/57278; or can differ by one or more amino acid residues in the region of overlap.

In a preferred embodiment, a 85041 fragment differs by at least 1, 2, 3, 10, 20, or more amino acid residues encoded by a sequence present in Genbank accession number AK022558, AK022818, or AF233321, or a sequence disclosed in WO 01/40466, WO 01/54472, WO 01/55318, or WO 01/53312. Differences can include differing in length or sequence identity. For example, a fragment can: include one or more amino acid residues from SEQ ID NO:26 outside the region encoded by nucleotides 55-2750, 1069-3010, 1347-3224, 1320-3010,1347-3010, 602-3005, 483-2740, 1015-1942, 786-3259, 602-3005, 883-2051, 2139-3010, 94-809, or 1843-3010 of SEQ ID NO:25; not include all of the amino acid residues encoded by a nucleotide sequence in Genbank accession number AK022558, AK022818, or AF233321, or a sequence disclosed in WO 01/40466, WO 01/54472, WO 01/55318, or WO 01/53312, e.g., can be one or more amino acid residues shorter (at one or both ends) than a sequence encoded by the nucleotide sequence in Genbank accession number AK022558, AK022818, or AF233321, or a sequence disclosed in WO 01/40466, WO 01/54472, WO 01/55318, or WO 01/53312; or can differ by one or more amino acid residues in the region of overlap.

In a preferred embodiment, a 84234 fragment differs by at least 1, 2, 3, 10, 20, or more amino acid residues encoded by a sequence disclosed in WO 01/53312. Differences can include differing in length or sequence identity. For example, a fragment can: not include all of the amino acid residues encoded by a sequence disclosed in WO 01/53312, e.g., can be one or more amino acid residues shorter (at one or both ends) than a sequence disclosed in WO 01/53312; or can differ by one or more amino acid residues in the region of overlap.

47476, 67210, 49875, 46842, 33201, 83378, 84233, 64708, 85041, or 84234 Chimeric or Fusion Proteins

In another aspect, the invention provides 47476, 67210, 49875, 46842, 33201, 83378, 84233, 64708, 85041, or 84234 chimeric or fusion proteins. As used herein, a 47476, 67210, 49875, 46842, 33201, 83378, 84233, 64708, 85041, or 84234 “chimeric protein” or “fusion protein” includes a 47476, 67210, 49875, 46842, 33201, 83378, 84233, 64708, 85041, or 84234 polypeptide linked to a non-47476, 67210, 49875, 46842, 33201, 83378, 84233, 64708, 85041, or 84234 polypeptide. A “non-47476, 67210, 49875, 46842, 33201, 83378, 84233, 64708, 85041, or 84234 polypeptide” refers to a polypeptide having an amino acid sequence corresponding to a protein which is not substantially homologous to the 47476, 67210, 49875, 46842, 33201, 83378, 84233, 64708, 85041, or 84234 protein, e.g., a protein which is different from the 47476, 67210, 49875, 46842, 33201, 83378, 84233, 64708, 85041, or 84234 protein and which is derived from the same or a different organism. The 47476, 67210, 49875, 46842, 33201, 83378, 84233, 64708, 85041, or 84234 polypeptide of the fusion protein can correspond to all or a portion e.g., a fragment described herein of a 47476, 67210, 49875, 46842, 33201, 83378, 84233, 64708, 85041, or 84234 amino acid sequence. In a preferred embodiment, a 47476, 67210, 49875, 46842, 33201, 83378, 84233, 64708, 85041, or 84234 fusion protein includes at least one (or two) biologically active portion of a 47476, 67210, 49875, 46842, 33201, 83378, 84233, 64708, 85041, or 84234 protein. The non-47476, 67210, 49875, 46842, 33201, 83378, 84233, 64708, 85041, or 84234 polypeptide can be fused to the N-terminus or C-terminus of the 47476, 67210, 49875, 46842, 33201, 83378, 84233, 64708, 85041, or 84234 polypeptide.

The fusion protein can include a moiety which has a high affinity for a ligand. For example, the fusion protein can be a GST-47476, 67210, 49875, 46842, 33201, 83378, 84233, 64708, 85041, or 84234 fusion protein in which the 47476, 67210, 49875, 46842, 33201, 83378, 84233, 64708, 85041, or 84234 sequences are fused to the C-terminus of the GST sequences. Such fusion proteins can facilitate the purification of recombinant 47476, 67210, 49875, 46842, 33201, 83378, 84233, 64708, 85041, or 84234. Alternatively, the fusion protein can be a 47476, 67210, 49875, 46842, 33201, 83378, 84233, 64708, 85041, or 84234 protein containing a heterologous signal sequence at its N-terminus. In certain host cells (e.g., mammalian host cells), expression and/or secretion of 47476, 67210, 49875, 46842, 33201, 83378, 84233, 64708, 85041, or 84234 can be increased through use of a heterologous signal sequence.

Fusion proteins can include all or a part of a serum protein, e.g., an IgG constant region, or human serum albumin.

The 47476, 67210, 49875, 46842, 33201, 83378, 84233, 64708, 85041, or 84234 fusion proteins of the invention can be incorporated into pharmaceutical compositions and administered to a subject in vivo. The 47476, 67210, 49875, 46842, 33201, 83378, 84233, 64708, 85041, or 84234 fusion proteins can be used to affect the bioavailability of a 47476, 67210, 49875, 46842, 33201, 83378, 84233, 64708, 85041, or 84234 substrate. 47476, 67210, 49875, 46842, 33201, 83378, 84233, 64708, 85041, or 84234 fusion proteins may be useful therapeutically for the treatment of disorders caused by, for example, (i) aberrant modification or mutation of a gene encoding a 47476, 67210, 49875, 46842, 33201, 83378, 84233, 64708, 85041, or 84234 protein; (ii) mis-regulation of the 47476, 67210, 49875, 46842, 33201, 83378, 84233, 64708, 85041, or 84234 gene; and (iii) aberrant post-translational modification of a 47476, 67210, 49875, 46842, 33201, 83378, 84233, 64708, 85041, or 84234 protein.

Moreover, the 47476, 67210, 49875, 46842, 33201, 83378, 84233, 64708, 85041, or 84234-fusion proteins of the invention can be used as immunogens to produce anti-47476, 67210, 49875, 46842, 33201, 83378, 84233, 64708, 85041, or 84234 antibodies in a subject, to purify 47476, 67210, 49875, 46842, 33201, 83378, 84233, 64708, 85041, or 84234 ligands and in screening assays to identify molecules which inhibit the interaction of 47476, 67210, 49875, 46842, 33201, 83378, 84233, 64708, 85041, or 84234 with a 47476, 67210, 49875, 46842, 33201, 83378, 84233, 64708, 85041, or 84234 substrate.

Expression vectors are commercially available that already encode a fusion moiety (e.g., a GST polypeptide). A 47476, 67210, 49875, 46842, 33201, 83378, 84233, 64708, 85041, or 84234-encoding nucleic acid can be cloned into such an expression vector such that the fusion moiety is linked in-frame to the 47476, 67210, 49875, 46842, 33201, 83378, 84233, 64708, 85041, or 84234 protein.

Variants of 47476, 67210, 49875, 46842, 33201, 83378, 84233, 64708, 85041, or 84234 Proteins

In another aspect, the invention also features a variant of a 47476, 67210, 49875, 46842, 33201, 83378, 84233, 64708, 85041, or 84234 polypeptide, e.g., which functions as an agonist (mimetics) or as an antagonist. Variants of the 47476, 67210, 49875, 46842, 33201, 83378, 84233, 64708, 85041, or 84234 proteins can be generated by mutagenesis, e.g., discrete point mutation, the insertion or deletion of sequences or the truncation of a 47476, 67210, 49875, 46842, 33201, 83378, 84233, 64708, 85041, or 84234 protein. An agonist of the 47476, 67210, 49875, 46842, 33201, 83378, 84233, 64708, 85041, or 84234 proteins can retain substantially the same, or a subset, of the biological activities of the naturally occurring form of a 47476, 67210, 49875, 46842, 33201, 83378, 84233, 64708, 85041, or 84234 protein. An antagonist of a 47476, 67210, 49875, 46842, 33201, 83378, 84233, 64708, 85041, or 84234 protein can inhibit one or more of the activities of the naturally occurring form of the 47476, 67210, 49875, 46842, 33201, 83378, 84233, 64708, 85041, or 84234 protein by, for example, competitively modulating a 47476, 67210, 49875, 46842, 33201, 83378, 84233, 64708, 85041, or 84234-mediated activity of a 47476, 67210, 49875, 46842, 33201, 83378, 84233, 64708, 85041, or 84234 protein. Thus, specific biological effects can be elicited by treatment with a variant of limited function. Preferably, treatment of a subject with a variant having a subset of the biological activities of the naturally occurring form of the protein has fewer side effects in a subject relative to treatment with the naturally occurring form of the 47476, 67210, 49875, 46842, 33201, 83378, 84233, 64708, 85041, or 84234 protein.

Variants of a 47476, 67210, 49875, 46842, 33201, 83378, 84233, 64708, 85041, or 84234 protein can be identified by screening combinatorial libraries of mutants, e.g., truncation mutants, of a 47476, 67210, 49875, 46842, 33201, 83378, 84233, 64708, 85041, or 84234 protein for agonist or antagonist activity.

Libraries of fragments e.g., N-terminal, C-terminal, or internal fragments of a 47476, 67210, 49875, 46842, 33201, 83378, 84233, 64708, 85041, or 84234 protein coding sequence can be used to generate a variegated population of fragments for screening and subsequent selection of variants of a 47476, 67210, 49875, 46842, 33201, 83378, 84233, 64708, 85041, or 84234 protein. Variants in which a cysteine residues is added or deleted or in which a residue which is glycosylated is added or deleted are particularly preferred.

Methods for screening gene products of combinatorial libraries made by point mutations or truncation, and for screening cDNA libraries for gene products having a selected property are known in the art. Such methods are adaptable for rapid screening of the gene libraries generated by combinatorial mutagenesis of 47476, 67210, 49875, 46842, 33201, 83378, 84233, 64708, 85041, or 84234 proteins. Recursive ensemble mutagenesis (REM), a new technique which enhances the frequency of functional mutants in the libraries, can be used in combination with the screening assays to identify 47476, 67210, 49875, 46842, 33201, 83378, 84233, 64708, 85041, or 84234 variants (Arkin and Yourvan (1992) Proc. Natl. Acad. Sci. USA 89:7811-7815; Delgrave et al. (1993) Protein Engineering 6:327-331).

Cell based assays can be exploited to analyze a variegated 47476 library. For example, a library of expression vectors can be transfected into a cell line, e.g., a cell line, which ordinarily responds to 47476 in a substrate-dependent manner. The transfected cells are then contacted with the substrate and the effect of the expression of the 47476 mutant on signaling by the substrate can be detected, e.g., by measuring GTP loading of a ras superfamily protein, measuring signal transduction that involves an activated ras superfamily protein, or by assaying for the changes induced by such signal transduction, e.g., cellular proliferation, differentiation, or migration. Plasmid DNA can then be recovered from the cells which score for inhibition, or alternatively, potentiation of signaling by the 47476 substrate, and the individual clones further characterized.

Cell based assays can be exploited to analyze a variegated 67210 library. For example, a library of expression vectors can be transfected into a cell line, e.g., a cell line, which ordinarily responds to 67210 in a substrate-dependent manner. The transfected cells are then contacted with the substrate and the effect of the expression of the 67210 mutant on signaling by the substrate can be detected, e.g., by measuring cellular properties influenced by 67210 activity, e.g., the appearance of particular glycosylated molecules on the cell surface, cellular adhesion, or signal transduction, e.g., as measured by cell proliferation or differentiation. Plasmid DNA can then be recovered from the cells which score for inhibition, or alternatively, potentiation of signaling by the 67210 substrate, and the individual clones further characterized.

Cell based assays can be exploited to analyze a variegated 49875 library. For example, a library of expression vectors can be transfected into a cell line, e.g., a cell line, which ordinarily responds to 49875 in a substrate-dependent manner. The transfected cells are then contacted with the substrate and the effect of the expression of the 49875 mutant on signaling by the substrate can be detected, e.g., by measuring a cellular process responsive to 49875 helicase activity, e.g., cellular proliferation or differentiation. Plasmid DNA can then be recovered from the cells which score for inhibition, or alternatively, potentiation of signaling by the 49875 substrate, and the individual clones further characterized.

Cell based assays can be exploited to analyze a variegated 46842 library. For example, a library of expression vectors can be transfected into a cell line, e.g., a cell line, which ordinarily responds to 46842 in a substrate-dependent manner. The transfected cells are then contacted with the substrate and the effect of the expression of the 46842 mutant on signaling by the substrate can be detected, e.g., by measuring GTP hydrolysis (e.g., ARF GTP hydrolysis), or cellular changes that reflect the presence or absence of Arf activity, e.g., changes in the transport of an appropriate marker protein, e.g., a cell surface or secreted marker protein. Plasmid DNA can then be recovered from the cells which score for inhibition, or alternatively, potentiation of signaling by the substrate, and the individual clones further characterized.

Cell based assays can be exploited to analyze a variegated 33201 library. For example, a library of expression vectors can be transfected into a cell line, e.g., a cell line, which ordinarily responds to 33201 in a substrate-dependent manner. The transfected cells are then contacted with the substrate and the effect of the expression of the 33201 mutant on signaling by the substrate can be detected, e.g., by measuring a cellular property dependent upon the substrate, e.g., cellular proliferation or cellular differentiation. Plasmid DNA can then be recovered from the cells which score for inhibition, or alternatively, potentiation of signaling by the substrate, and the individual clones further characterized.

Cell based assays can be exploited to analyze a variegated 83378, 84233, 64708, 85041, or 84234 library. For example, a library of expression vectors can be transfected into a cell line, e.g., a cell line, which ordinarily responds to 83378, 84233, 64708, 85041, or 84234 in a substrate-dependent manner. The transfected cells are then contacted with the sustrate and the effect of the expression of the 83378, 84233, 64708, 85041, or 84234 mutant on signaling by the substrate can be detected, e.g., by measuring cation diffusion or a response of the cells to the substrate, e.g., a normal cellular response such as programmed cell death. Plasmid DNA can then be recovered from the cells which score for inhibition, or alternatively, potentiation of signaling by the substrate, and the individual clones further characterized.

In some embodiments, the cell based assays decribed above do not require the addition of a substrate, e.g., the cell may respond to simply the expression of the 47476, 67210, 49875, 46842, 33201, 83378, 84233, 64708, 85041, or 84234 polypeptide or protein. In such cases, the assay can be modified such that the 47476, 67210, 49875, 46842, 33201, 83378, 84233, 64708, 85041, or 84234 polypeptides or proteins are expressed by an inducible promoter.

In another aspect, the invention features a method of making a 47476, 67210, 49875, 46842, 33201, 83378, 84233, 64708, 85041, or 84234 polypeptide, e.g., a peptide having a non-wild type activity, e.g., an antagonist, agonist, or super agonist of a naturally occurring 47476, 67210, 49875, 46842, 33201, 83378, 84233, 64708, 85041, or 84234 polypeptide, e.g., a naturally occurring 47476, 67210, 49875, 46842, 33201, 83378, 84233, 64708, 85041, or 84234 polypeptide. The method includes: altering the sequence of a 47476, 67210, 49875, 46842, 33201, 83378, 84233, 64708, 85041, or 84234 polypeptide, e.g., altering the sequence, e.g., by substitution or deletion of one or more residues of a non-conserved region, a domain or residue disclosed herein, and testing the altered polypeptide for the desired activity.

In another aspect, the invention features a method of making a fragment or analog of a 47476, 67210, 49875, 46842, 33201, 83378, 84233, 64708, 85041, or 84234 polypeptide a biological activity of a naturally occurring 47476, 67210, 49875, 46842, 33201, 83378, 84233, 64708, 85041, or 84234 polypeptide. The method includes: altering the sequence, e.g., by substitution or deletion of one or more residues, of a 47476, 67210, 49875, 46842, 33201, 83378, 84233, 64708, 85041, or 84234 polypeptide, e.g., altering the sequence of a non-conserved region, or a domain or residue described herein, and testing the altered polypeptide for the desired activity.

Anti-47476, 67210, 49875, 46842, 33201, 83378, 84233, 64708, 85041, or 84234 Antibodies

In another aspect, the invention provides an anti-47476, 67210, 49875, 46842, 33201, 83378, 84233, 64708, 85041, or 84234 antibody, or a fragment thereof (e.g., an antigen-binding fragment thereof). The term “antibody” as used herein refers to an immunoglobulin molecule or immunologically active portion thereof, i.e., an antigen-binding portion. As used herein, the term “antibody” refers to a protein comprising at least one, and preferably two, heavy (H) chain variable regions (abbreviated herein as VH), and at least one and preferably two light (L) chain variable regions (abbreviated herein as VL). The VH and VL regions can be further subdivided into regions of hypervariability, termed “complementarity determining regions” (“CDR”), interspersed with regions that are more conserved, termed “framework regions” (FR). The extent of the framework region and CDR's has been precisely defined (see, Kabat, E. A., et al. (1991) Sequences of Proteins of Immunological Interest, Fifth Edition, U.S. Department of Health and Human Services, NIH Publication No. 91-3242, and Chothia, C. et al. (1987) J. Mol. Biol. 196:901-917, which are incorporated herein by reference). Each VH and VL is composed of three CDR's and four FRs, arranged from amino-terminus to carboxy-terminus in the following order: FR1, CDR1, FR2, CDR2, FR3, CDR3, FR4.

The anti-47476, 67210, 49875, 46842, 33201, 83378, 84233, 64708, 85041, or 84234 antibody can further include a heavy and light chain constant region, to thereby form a heavy and light immunoglobulin chain, respectively. In one embodiment, the antibody is a tetramer of two heavy immunoglobulin chains and two light immunoglobulin chains, wherein the heavy and light immunoglobulin chains are inter-connected by, e.g., disulfide bonds. The heavy chain constant region is comprised of three domains, CH1, CH2 and CH3. The light chain constant region is comprised of one domain, CL. The variable region of the heavy and light chains contains a binding domain that interacts with an antigen. The constant regions of the antibodies typically mediate the binding of the antibody to host tissues or factors, including various cells of the immune system (e.g., effector cells) and the first component (C1q) of the classical complement system.

As used herein, the term “immunoglobulin” refers to a protein consisting of one or more polypeptides substantially encoded by immunoglobulin genes. The recognized human immunoglobulin genes include the kappa, lambda, alpha (IgA1 and IgA2), gamma (IgG1, IgG2, IgG3, IgG4), delta, epsilon and mu constant region genes, as well as the myriad immunoglobulin variable region genes. Full-length immunoglobulin “light chains” (about 25 KDa or 214 amino acids) are encoded by a variable region gene at the NH2-terminus (about 110 amino acids) and a kappa or lambda constant region gene at the COOH—terminus. Full-length immunoglobulin “heavy chains” (about 50 KDa or 446 amino acids), are similarly encoded by a variable region gene (about 116 amino acids) and one of the other aforementioned constant region genes, e.g., gamma (encoding about 330 amino acids).

The term “antigen-binding fragment” of an antibody (or simply “antibody portion,” or “fragment”), as used herein, refers to one or more fragments of a full-length antibody that retain the ability to specifically bind to the antigen, e.g., 47476, 67210, 49875, 46842, 33201, 83378, 84233, 64708, 85041, or 84234 polypeptide or fragment thereof. Examples of antigen-binding fragments of the anti-47476, 67210, 49875, 46842, 33201, 83378, 84233, 64708, 85041, or 84234 antibody include, but are not limited to: (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also encompassed within the term “antigen-binding fragment” of an antibody. These antibody fragments are obtained using conventional techniques known to those with skill in the art, and the fragments are screened for utility in the same manner as are intact antibodies.

The anti-47476, 67210, 49875, 46842, 33201, 83378, 84233, 64708, 85041, or 84234 antibody can be a polyclonal or a monoclonal antibody. In other embodiments, the antibody can be recombinantly produced, e.g., produced by phage display or by combinatorial methods.

Phage display and combinatorial methods for generating anti-47476, 67210, 49875, 46842, 33201, 83378, 84233, 64708, 85041, or 84234 antibodies are known in the art (as described in, e.g., Ladner et al. U.S. Pat. No. 5,223,409; Kang et al. International Publication No. WO 92/18619; Dower et al. International Publication No. WO 91/17271; Winter et al. International Publication WO 92/20791; Markland et al. International Publication No. WO 92/15679; Breitling et al. International Publication WO 93/01288; McCafferty et al. International Publication No. WO 92/01047; Garrard et al. International Publication No. WO 92/09690; Ladner et al. International Publication No. WO 90/02809; Fuchs et al. (1991) Bio/Technology 2:1370-1372; Hay et al. (1992) Hum Antibod Hybridomas 3:81-85; Huse et al. (1989) Science 246:1275-1281; Griffths et al. (1993) EMBO J. 12:725-734; Hawkins et al. (1992) J Mol Biol 226:889-896; Clackson et al. (1991) Nature 352:624-628; Gram et al. (1992) PNAS 89:3576-3580; Garrad et al. (1991) Bio/Technology 9:1373-1377; Hoogenboom et al. (1991) Nuc Acid Res 19:4133-4137; and Barbas et al. (1991) PNAS 88:7978-7982, the contents of all of which are incorporated by reference herein).

In one embodiment, the anti-47476, 67210, 49875, 46842, 33201, 83378, 84233, 64708, 85041, or 84234 antibody is a fully human antibody (e.g., an antibody made in a mouse which has been genetically engineered to produce an antibody from a human immunoglobulin sequence), or a non-human antibody, e.g., a rodent (mouse or rat), goat, primate (e.g., monkey), camel antibody. Preferably, the non-human antibody is a rodent (mouse or rat antibody). Method of producing rodent antibodies are known in the art.

Human monoclonal antibodies can be generated using transgenic mice carrying the human immunoglobulin genes rather than the mouse system. Splenocytes from these transgenic mice immunized with the antigen of interest are used to produce hybridomas that secrete human mAbs with specific affinities for epitopes from a human protein (see, e.g., Wood et al. International Application WO 91/00906, Kucherlapati et al. PCT publication WO 91/10741; Lonberg et al. International Application WO 92/03918; Kay et al. International Application 92/03917; Lonberg, N. et al. 1994 Nature 368:856-859; Green, L. L. et al. 1994 Nature Genet. 7:13-21; Morrison, S. L. et al. 1994 Proc. Natl. Acad. Sci. USA 81:6851-6855; Bruggeman et al. 1993 Year Immunol 7:33-40; Tuaillon et al. 1993 PNAS 90:3720-3724; Bruggeman et al. 1991 Eur J Immunol 21:1323-1326).

An anti-47476, 67210, 49875, 46842, 33201, 83378, 84233, 64708, 85041, or 84234 antibody can be one in which the variable region, or a portion thereof, e.g., the CDR's, are generated in a non-human organism, e.g., a rat or mouse. Chimeric, CDR-grafted, and humanized antibodies are within the invention. Antibodies generated in a non-human organism, e.g., a rat or mouse, and then modified, e.g., in the variable framework or constant region, to decrease antigenicity in a human are within the invention.

Chimeric antibodies can be produced by recombinant DNA techniques known in the art. For example, a gene encoding the Fc constant region of a murine (or other species) monoclonal antibody molecule is digested with restriction enzymes to remove the region encoding the murine Fc, and the equivalent portion of a gene encoding a human Fe constant region is substituted (see Robinson et al., International Patent Publication PCT/US86/02269; Akira, et al., European Patent Application 184,187; Taniguchi, M., European Patent Application 171,496; Morrison et al., European Patent Application 173,494; Neuberger et al., International Application WO 86/01533; Cabilly et al. U.S. Pat. No. 4,816,567; Cabilly et al., European Patent Application 125,023; Better et al. (1988 Science 240:1041-1043); Liu et al. (1987) PNAS 84:3439-3443; Liu et al., 1987, J. Immunol. 139:3521-3526; Sun et al. (1987) PNAS 84:214-218; Nishimura et al., 1987, Canc. Res. 47:999-1005; Wood et al. (1985) Nature 314:446-449; and Shaw et al., 1988, J. Natl Cancer Inst. 80:1553-1559).

A humanized or CDR-grafted antibody will have at least one or two but generally all three recipient CDR's (of heavy and or light immuoglobulin chains) replaced with a donor CDR. The antibody may be replaced with at least a portion of a non-human CDR or only some of the CDR's may be replaced with non-human CDR's. It is only necessary to replace the number of CDR's required for binding of the humanized antibody to a 47476, 67210, 49875, 46842, 33201, 83378, 84233, 64708, 85041, or 84234 or a fragment thereof. Preferably, the donor will be a rodent antibody, e.g., a rat or mouse antibody, and the recipient will be a human framework or a human consensus framework. Typically, the immunoglobulin providing the CDR's is called the “donor” and the immunoglobulin providing the framework is called the “acceptor.” In one embodiment, the donor immunoglobulin is a non-human (e.g., rodent). The acceptor framework is a naturally-occurring (e.g., a human) framework or a consensus framework, or a sequence about 85% or higher, preferably 90%, 95%, 99% or higher identical thereto.

As used herein, the term “consensus sequence” refers to the sequence formed from the most frequently occurring amino acids (or nucleotides) in a family of related sequences (See e.g., Winnaker, From Genes to Clones (Verlagsgesellschaft, Weinheim, Germany 1987). In a family of proteins, each position in the consensus sequence is occupied by the amino acid occurring most frequently at that position in the family. If two amino acids occur equally frequently, either can be included in the consensus sequence. A “consensus framework” refers to the framework region in the consensus immunoglobulin sequence.

An antibody can be humanized by methods known in the art. Humanized antibodies can be generated by replacing sequences of the Fv variable region which are not directly involved in antigen binding with equivalent sequences from human Fv variable regions. General methods for generating humanized antibodies are provided by Morrison, S. L., 1985, Science 229:1202-1207, by Oi et al., 1986, BioTechniques 4:214, and by Queen et al. U.S. Pat. No. 5,585,089, U.S. Pat. No. 5,693,761 and U.S. Pat. No. 5,693,762, the contents of all of which are hereby incorporated by reference. Those methods include isolating, manipulating, and expressing the nucleic acid sequences that encode all or part of immunoglobulin Fv variable regions from at least one of a heavy or light chain. Sources of such nucleic acid are well known to those skilled in the art and, for example, may be obtained from a hybridoma producing an antibody against a 47476, 67210, 49875, 46842, 33201, 83378, 84233, 64708, 85041, or 84234 polypeptide or fragment thereof. The recombinant DNA encoding the humanized antibody, or fragment thereof, can then be cloned into an appropriate expression vector.

Humanized or CDR-grafted antibodies can be produced by CDR-grafting or CDR substitution, wherein one, two, or all CDR's of an immunoglobulin chain can be replaced. See e.g., U.S. Pat. No. 5,225,539; Jones et al. 1986 Nature 321:552-525; Verhoeyan et al. 1988 Science 239:1534; Beidler et al. 1988 J. Immunol. 141:4053-4060; Winter U.S. Pat. No. 5,225,539, the contents of all of which are hereby expressly incorporated by reference. Winter describes a CDR-grafting method which may be used to prepare the humanized antibodies of the present invention (UK Patent Application GB 2188638A, filed on Mar. 26, 1987; Winter U.S. Pat. No. 5,225,539), the contents of which is expressly incorporated by reference.

Also within the scope of the invention are humanized antibodies in which specific amino acids have been substituted, deleted or added. Preferred humanized antibodies have amino acid substitutions in the framework region, such as to improve binding to the antigen. For example, a humanized antibody will have framework residues identical to the donor framework residue or to another amino acid other than the recipient framework residue. To generate such antibodies, a selected, small number of acceptor framework residues of the humanized immunoglobulin chain can be replaced by the corresponding donor amino acids. Preferred locations of the substitutions include amino acid residues adjacent to the CDR, or which are capable of interacting with a CDR (see e.g., U.S. Pat. No. 5,585,089). Criteria for selecting amino acids from the donor are described in U.S. Pat. No. 5,585,089, e.g., columns 12-16 of U.S. Pat. No. 5,585,089, the e.g., columns 12-16 of U.S. Pat. No. 5,585,089, the contents of which are hereby incorporated by reference. Other techniques for humanizing antibodies are described in Padlan et al. EP 519596 A1, published on Dec. 23, 1992.

In preferred embodiments an antibody can be made by immunizing with purified 47476, 67210, 49875, 46842, 33201, 83378, 84233, 64708, 85041, or 84234 antigen, or a fragment thereof, e.g., a fragment described herein, membrane associated antigen, tissue, e.g., crude tissue preparations, whole cells, preferably living cells, lysed cells, or cell fractions, e.g., cytosolic fractions or membrane fractions.

A full-length 47476, 67210, 49875, 46842, 33201, 83378, 84233, 64708, 85041, or 84234 protein or, antigenic peptide fragment of 47476, 67210, 49875, 46842, 33201, 83378, 84233, 64708, 85041, or 84234 can be used as an immunogen or can be used to identify anti-47476, 67210, 49875, 46842, 33201, 83378, 84233, 64708, 85041, or 84234 antibodies made with other immunogens, e.g., cells, membrane preparations, and the like. The antigenic peptide of 47476, 67210, 49875, 46842, 33201, 83378, 84233, 64708, 85041, or 84234 should include at least 8 amino acid residues of the amino acid sequence shown in SEQ ID NO:2, SEQ ID NO:5, SEQ ID NO:8, SEQ ID NO:11, SEQ ID NO:14, SEQ ID NO:17, SEQ ID NO:20, SEQ ID NO:23, SEQ ID NO:26, or SEQ ID NO:29 and encompasses an epitope of 47476, 67210, 49875, 46842, 33201, 83378, 84233, 64708, 85041, or 84234. Preferably, the antigenic peptide includes at least 10 amino acid residues, more preferably at least 15 amino acid residues, even more preferably at least 20 amino acid residues, and most preferably at least 30 amino acid residues.

Fragments of 47476 can be used as immunogens or used to characterize the specificity of an antibody. For example, fragments of 47476 that include, e.g., residues about 106 to 122, about 325 to 340, or about 500 to 520 of SEQ ID NO:2, can be used to makeantibodies against hydrophilic regions of the 47476 protein. Similarly, fragments of 47476 which include, e.g., residues about 82 to 105, about 341 to 360, or about 521 to 540 of SEQ ID NO:2, can be used to make an antibody against a hydrophobic region of the 47476 protein; fragments of 47476, which include, e.g., residues about 55 to 173, about 195 to 381, about 470 to 498, or about 541 to 590 of SEQ ID NO:2, can be used to make an antibody against the a guanine nucleotide dissociation stimulator domain N-terminal motif, a ras guanine nucleotide dissociation stimulator domain, an EF-hand calcium-binding domain and a phorbol ester/diacylglycerol binding domain (C1 domain), respectively, of the 47476 protein.

Fragments of 67210 can be used as immunogens or used to characterize the specificity of an antibody. For example, fragments of 67210 which include, e.g., residues about 240-260, can be used to make antibodies against hydrophilic regions of the 67210 protein. Similarly, fragments of 67210 which include, e.g., residues about 10-25 of SEQ ID NO:5, can be used to make an antibody against a hydrophobic region of the 67210 protein; and fragments of 67210 which include, e.g., residues about 63 to 340 of SEQ ID NO:5, can be used to make an antibody against the glycosyltransferase region of the 67210 protein.

Fragments of 49875 can be used as immunogens or used to characterize the specificity of an antibody. For example, fragments of 49875 which include, e.g., residues about 520 to 550 of SEQ ID NO:8, can be used to make antibodies against hydrophilic regions of the 49875 protein. Similarly, fragments of 49875 which include, e.g., residues about 285 to 295 of SEQ ID NO:8, can be used to make an antibody against a hydrophobic region of the 49875 protein; fragments of 49875 which include, e.g., residues about 22 to 245 of SEQ ID NO:8, can be used to make an antibody against the DEAD-type helicase region of the 49875 protein; fragments of 49875 which include, e.g., about amino acid residues 169 to 177 of SEQ ID NO:8, can be used to maka an antibody against a DEAD-box subfamily ATP-dependent helicase signature motif; and fragments of 49875 which include, e.g., residues about 281 to 363 of SEQ ID NO:8, can be used to make an antibody against the conserved helicase C-terminal region of the 49875 protein.

Fragments of 46842 can be used as immunogens or used to characterize the specificity of an antibody. For example, fragments of 46842 which include, e.g., residues about 567 to 580, 720 to 737, or 757 to 771 of SEQ ID NO:11, can be used to make antibodies against hydrophilic regions of the 46842 protein. Similarly, fragments of 46842 which include, e.g., residues about 431 to 439, from about 558 to 566, and from about 706 to 719 of SEQ ID NO:11, can be used to make an antibody against a hydrophobic region of the 46842 protein; fragments of 46842 which include, e.g., residues about 269 to 363 of SEQ ID NO:11, can be used to make an antibody against the PH domain region of the 46842 protein; fragments of 46842 which include, e.g., residues about 403 to 525 of SEQ ID NO:11, can be used to make an antibody against the ArfGAP domain region of the 46842 protein; and fragments of 46842 which include, e.g., residues about 702 to 734 or 735 to 767 of SEQ ID NO:11, can be used to make an antibody against the ankyrin repeat domains of 46842.

Fragments of 33201 can be used, e.g., to characterize the specificity of an antibody or to make immunogens. For example, fragments of 33201 which include, e.g., residues about 50 to 60, 82 to 90, or 205 to 210 of SEQ ID NO:14, can be used to make antibodies against hydrophilic regions of the 33201 protein. Similarly, fragments of 33201 which include, e.g., residues about 70 to 80 or 158 to 178 of SEQ ID NO:14, can be used to make an antibody against a hydrophobic region of the 33201 protein; fragments of 33201 which include residues from about 22 to 345 of SEQ ID NO:14, or a fragment thereof, e.g., 22 to 50, 50 to 100, 150 to 200, 250 to 300, or 300 to 345 of SEQ ID NO:14, can be used to make an antibody against a dehydrogenase/reductase domain of the 33201 protein.

Fragments of 83378, 84233, 64708, 85041, or 84234 can be used, e.g., as immunogens or to characterize the specificity of an antibody. For example, fragments of 83378, 84233, 64708, 85041, or 84234 which include about amino acid residues 150 to 160, 220 to 235, or 355 to 370 of SEQ ID NO:17, about amino acid residues 1 to 20, 80 to 90, or 150 to 160 of SEQ ID NO:20, about amino acid residues 123 to 133, 380 to 395, or 450 to 461 of SEQ ID NO:23, about amino acid residues 320 to 340, 555 to 575, or 750 to 765 of SEQ ID NO:26, or about amino acid residues 10 to 20, 165 to 175, or 190 to 230 of SEQ ID NO:29, can be used to make antibodies against hydrophilic regions of the 83378, 84233, 64708, 85041, or 84234 protein.

Similarly fragments of 83378, 84233, 64708, 85041, or 84234 which include residues about amino acid residues 325 to 335, 340 to 350, or 415 to 430 of SEQ ID NO:17, about amino acid residues 220 to 230, 240 to 260, or 262 to 273 of SEQ ID NO:20, about amino acid residues 180 to 195, 290 to 300, or 340 to 350 of SEQ ID NO:23, about amino acid residues 35 to 50, 440 to 470, or 685 to 695 of SEQ ID NO:26, or about amino acid residues 59 to 70, 330 to 345, or 370 to 376 of SEQ ID NO:29 can be used to make an antibody against a hydrophobic region of the 83378, 84233, 64708, 85041, or 84234 protein.

Fragments of 83378, 84233, 64708, 85041, or 84234 which include about amino acid residues 32 to 43, 99 to 114, or 266 to 282 of SEQ ID NO:17, about amino acid residues 50 to 57, 114 to 127, or 192 to 200 of SEQ ID NO:20, about amino acid residues 52 to 57, 120 to 136, or 220 to 231 of SEQ ID NO:23, about amino acid residues 78 to 98, 146 to 151, 215 to 238, 289 to 303, 363 to 418, 506 to 520, or 614 to 617 of SEQ ID NO:26, or about amino acid residues 59 to 70, 124 to 140, or 257 to 262 of SEQ ID NO:29 can be used to make an antibody against a non-cytoplasmic loop of the 83378, 84233, 64708, 85041, or 84234 protein.

Fragments of 83378, 84233, 64708, 85041, or 84234 which include about amino acid residues 1 to 10, 62 to 78, 135 to 240, or 300 to 485 of SEQ ID NO:17, about amino acid residues 1 to 24, 75 to 91, 148 to 166, or 219 to 320 of SEQ ID NO:20, about amino acid residues 1 to 33, 83 to 100, 156 to 201, or 250 to 461 of SEQ ID NO:23, about amino acid residues 1 to 58, 120 to 128, 169 to 189, 259 to 266, 321 to 342, 438 to 485, 542 to 591, or 642 to 765 of SEQ ID NO:26, or about amino acid residues 1 to 37, 88 to 104, 160 to 236, or 287 to 376 of SEQ ID NO:29 can be used to make an antibody against a cytoplasmic domain of the 83378, 84233, 64708, 85041, or 84234 protein.

Fragment of 83378, 84233, 64708, 85041, or 84234 which include about amino acid residues 11 to 133 or 231 to 389 of SEQ ID NO:17, about amino acid residues 25 to 310 of SEQ ID NO:20, about amino acid residues 55 to 153 or 227 to 320 of SEQ ID NO:23, about amino acid residues 419 to 733 of SEQ ID NO:26, or about amino acid residues 38 to 349 of SEQ ID NO:29 can be used to make an antibody against the cation efflux region of the 83378, 84233, 64708, 85041, or 84234 protein.

Antibodies reactive with, or specific for, any of these regions, or other regions or domains described herein are provided.

Antibodies which bind only native 47476, 67210, 49875, 46842, 33201, 83378, 84233, 64708, 85041, or 84234 protein, only denatured or otherwise non-native 47476, 67210, 49875, 46842, 33201, 83378, 84233, 64708, 85041, or 84234 protein, or which bind both, are with in the invention. Antibodies with linear or conformational epitopes are within the invention. Conformational epitopes can sometimes be identified by identifying antibodies which bind to native but not denatured 47476, 67210, 49875, 46842, 33201, 83378, 84233, 64708, 85041, or 84234 protein.

Preferred epitopes encompassed by the antigenic peptide are regions of 47476, 67210, 49875, 46842, 33201, 83378, 84233, 64708, 85041, or 84234 are located on the surface of the protein, e.g., hydrophilic regions, as well as regions with high antigenicity. For example, an Emini surface probability analysis of the human 47476, 67210, 49875, 46842, 33201, 83378, 84233, 64708, 85041, or 84234 protein sequence can be used to indicate the regions that have a particularly high probability of being localized to the surface of the 47476, 67210, 49875, 46842, 33201, 83378, 84233, 64708, 85041, or 84234 protein.

In a preferred embodiment the antibody can bind to the extracellular portion of a 67210, 83378, 84233, 64708, 85041, or 84234 protein, e.g., it can bind to a whole cell which expresses a 67210, 83378, 84233, 64708, 85041, or 84234 protein. In another embodiment, the antibody binds an intracellular portion of a 47476, 67210, 49875, 46842, 33201, 83378, 84233, 64708, 85041, or 84234 protein. In preferred embodiments antibodies can bind one or more of purified antigen, membrane associated antigen, tissue, e.g., tissue sections, whole cells, preferably living cells, lysed cells, cell fractions, e.g., cytosolic fractions or membrane fractions.

The anti-47476, 67210, 49875, 46842, 33201, 83378, 84233, 64708, 85041, or 84234 antibody can be a single chain antibody. A single-chain antibody (scFV) may be engineered (see, for example, Colcher, D. et al. (1999) Ann NY Acad Sci 880:263-80; and Reiter, Y. (1996) Clin Cancer Res 2:245-52). The single chain antibody can be dimerized or multimerized to generate multivalent antibodies having specificities for different epitopes of the same target 47476, 67210, 49875, 46842, 33201, 83378, 84233, 64708, 85041, or 84234 protein.

In a preferred embodiment the antibody has effector function and/or can fix complement. In other embodiments the antibody does not recruit effector cells; or fix complement.

In a preferred embodiment, the antibody has reduced or no ability to bind an Fe receptor. For example, it is a isotype or subtype, fragment or other mutant, which does not support binding to an Fe receptor, e.g., it has a mutagenized or deleted Fe receptor binding region.

In a preferred embodiment, an anti-47476 antibody alters (e.g., increases or decreases) that ability of 47476 to: (1) stimulate the exchange of guanine nucleotides (GTP for GDP) by a member of the ras superfamily of proteins; (2) bind calcium in a pentagonal bipyramidal configuration; (3) bind two zinc ions; (4) bind the second messenger diacylglycerol; (5) bind analogs of diacylglycerol, such as phorbol esters; or (6) activate one or more members of the ras superfamily of proteins: For example, the antibody can bind at or in proximity to the active site of a 47476 polypeptide or protein, e.g., to an epitope that is present within: a guanine nucleotide dissociation stimulator domain N-terminal motif, e.g., located at about amino acid residues 55 to 172 of SEQ ID NO:2; a guanine nucleotide dissociation stimulator domain, e.g., located at about amino acid residues 195 to 381 of SEQ ID NO:2; an EF-hand calcium-binding domain, e.g., located at about amino acid residues 470 to 498 of SEQ ID NO:2; or a phorbol ester/diacylglycerol binding domain (C1 domain), e.g., located at about amino acid residues 541 to 590 of SEQ ID NO:2.

In a preferred embodiment, an anti-67210 antibody alters (e.g., increases or decreases) the glycosyltransferase activity of a 67210 polypeptide. For example, the antibody can bind at or in proximity to the active site of a 67210 polypeptide or protein, e.g., to an epitope that is present within the glycosyl transferase domain, e.g., located at about amino acid residues 63 to 340 of SEQ ID NO:5.

In a preferred embodiment, an anti-49875 antibody alters (e.g., increases or decreases) the nucleic acid unwinding activity of a 49875 polypeptide. For example, the antibody can bind at or in proximity to the active site, e.g., to an epitope that includes a DEAD type helicase ATP binding motif, e.g., about amino acid residues 169 to 177 of SEQ ID NO:8.

In a preferred embodiment, an anti-46842 antibody alters (e.g., increases or decreases) the ArfGAP activity of a 46842 polypeptide. For example, the antibody can bind at or in proximity to a motif involved in ArfGAP catalytic activity, e.g., the sequence located at about residues 421 to 440 of SEQ ID NO:11.

In a preferred embodiment, an anti-33201 antibody alters (e.g., increases or decreases) the dehydrogenase/reductase activity of a 33201 polypeptide. For example, the antibody can bind at or in proximity to the active site, e.g., to an epitope that includes a residue located within a region from about 22 to 345 of SEQ ID NO:14, or a fragment thereof, e.g., 100 to 150, 150 to 200, 250 to 300, or 300 to 335 of SEQ ID NO:14.

In a preferred embodiment, an anti-83378, 84233, 64708, 85041, or 84234 antibody alters (e.g., increases or decreases) the cation diffusion activity of a 83378, 84233, 64708, 85041, or 84234 polypeptide.

The antibody can be coupled to a toxin, e.g., a polypeptide toxin, e,g, ricin or diphtheria toxin or active fragment hereof, or a radioactive nucleus, or imaging agent, e.g. a radioactive, enzymatic, or other, e.g., imaging agent, e.g., a NMR contrast agent. Labels which produce detectable radioactive emissions or fluorescence are preferred.

An anti-47476, 67210, 49875, 46842, 33201, 83378, 84233, 64708, 85041, or 84234 antibody (e.g., monoclonal antibody) can be used to isolate 47476, 67210, 49875, 46842, 33201, 83378, 84233, 64708, 85041, or 84234 by standard techniques, such as affinity chromatography or immunoprecipitation. Moreover, an anti-47476, 67210, 49875, 46842, 33201, 83378, 84233, 64708, 85041, or 84234 antibody can be used to detect 47476, 67210, 49875, 46842, 33201, 83378, 84233, 64708, 85041, or 84234 protein (e.g., in a cellular lysate or cell supernatant) in order to evaluate the abundance and pattern of expression of the protein. Anti-47476, 67210, 49875, 46842, 33201, 83378, 84233, 64708, 85041, or 84234 antibodies can be used diagnostically to monitor protein levels in tissue as part of a clinical testing procedure, e.g., to determine the efficacy of a given treatment regimen. Detection can be facilitated by coupling (i.e., physically linking) the antibody to a detectable substance (i.e., antibody labelling). Examples of detectable substances include various enzymes, prosthetic groups, fluorescent materials, luminescent materials, bioluminescent materials, and radioactive materials. Examples of suitable enzymes include horseradish peroxidase, alkaline phosphatase, βgalactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

The invention also includes a nucleic acid which encodes an anti-47476, 67210, 49875, 46842, 33201, 83378, 84233, 64708, 85041, or 84234 antibody, e.g., an anti-47476, 67210, 49875, 46842, 33201, 83378, 84233, 64708, 85041, or 84234 antibody described herein. Also included are vectors which include the nucleic acid and cells transformed with the nucleic acid, particularly cells which are useful for producing an antibody, e.g., mammalian cells, e.g. CHO or lymphatic cells.

The invention also includes cell lines, e.g., hybridomas, which make an anti-47476, 67210, 49875, 46842, 33201, 83378, 84233, 64708, 85041, or 84234 antibody, e.g., an antibody described herein, and method of using said cells to make a 47476, 67210, 49875, 46842, 33201, 83378, 84233, 64708, 85041, or 84234 antibody.

47476, 67210, 49875, 46842, 33201, 83378, 84233, 64708, 85041, and 84234 Recombinant Expression Vectors, Host Cells and Genetically Engineered Cells

In another aspect, the invention includes, vectors, preferably expression vectors, containing a nucleic acid encoding a polypeptide described herein. As used herein, the term “vector” refers to a nucleic acid molecule capable of transporting another nucleic acid to which it has been linked and can include a plasmid, cosmid or viral vector. The vector can be capable of autonomous replication or it can integrate into a host DNA. Viral vectors include, e.g., replication defective retroviruses, adenoviruses and adeno-associated viruses.

A vector can include a 47476, 67210, 49875, 46842, 33201, 83378, 84233, 64708, 85041, or 84234 nucleic acid in a form suitable for expression of the nucleic acid in a host cell. Preferably the recombinant expression vector includes one or more regulatory sequences operatively linked to the nucleic acid sequence to be expressed. The term “regulatory sequence” includes promoters, enhancers and other expression control elements (e.g., polyadenylation signals). Regulatory sequences include those which direct constitutive expression of a nucleotide sequence, as well as tissue-specific regulatory and/or inducible sequences. The design of the expression vector can depend on such factors as the choice of the host cell to be transformed, the level of expression of protein desired, and the like. The expression vectors of the invention can be introduced into host cells to thereby produce proteins or polypeptides, including fusion proteins or polypeptides, encoded by nucleic acids as described herein (e.g., 47476, 67210, 49875, 46842, 33201, 83378, 84233, 64708, 85041, or 84234 proteins, mutant forms of 47476, 67210, 49875, 46842, 33201, 83378, 84233, 64708, 85041, or 84234 proteins, fusion proteins, and the like).

The recombinant expression vectors of the invention can be designed for expression of 47476, 67210, 49875, 46842, 33201, 83378, 84233, 64708, 85041, or 84234 proteins in prokaryotic or eukaryotic cells. For example, polypeptides of the invention can be expressed in E. coli, insect cells (e.g., using baculovirus expression vectors), yeast cells or mammalian cells. Suitable host cells are discussed further in Goeddel, (1990) Gene Expression Technology: Methods in Enzymology 185, Academic Press, San Diego, Calif. Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith, D. B. and Johnson, K. S. (1988) Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

Purified fusion proteins can be used in 47476, 67210, 49875, 46842, 33201, 83378, 84233, 64708, 85041, or 84234 activity assays, (e.g., direct assays or competitive assays described in detail below), or to generate antibodies specific for 47476, 67210, 49875, 46842, 33201, 83378, 84233, 64708, 85041, or 84234 proteins. In a preferred embodiment, a fusion protein expressed in a retroviral expression vector of the present invention can be used to infect bone marrow cells which are subsequently transplanted into irradiated recipients. The pathology of the subject recipient is then examined after sufficient time has passed (e.g., six weeks).

To maximize recombinant protein expression in E. coli is to express the protein in a host bacteria with an impaired capacity to proteolytically cleave the recombinant protein (Gottesman, S., (1990) Gene Expression Technology: Methods in Enzymology 185, Academic Press, San Diego, Calif. 119-128). Another strategy is to alter the nucleic acid sequence of the nucleic acid to be inserted into an expression vector so that the individual codons for each amino acid are those preferentially utilized in E. coli (Wada et al., (1992) Nucleic Acids Res. 20:2111-2118). Such alteration of nucleic acid sequences of the invention can be carried out by standard DNA synthesis techniques.

The 47476, 67210, 49875, 46842, 33201, 83378, 84233, 64708, 85041, or 84234 expression vector can be a yeast expression vector, a vector for expression in insect cells, e.g., a baculovirus expression vector or a vector suitable for expression in mammalian cells.

When used in mammalian cells, the expression vector's control functions can be provided by viral regulatory elements. For example, commonly used promoters are derived from polyoma, Adenovirus 2, cytomegalovirus and Simian Virus 40.

In another embodiment, the promoter is an inducible promoter, e.g., a promoter regulated by a steroid hormone, by a polypeptide hormone (e.g., by means of a signal transduction pathway), or by a heterologous polypeptide (e.g., the tetracycline-inducible systems, “Tet-On” and “Tet-Off”; see, e.g., Clontech Inc., CA, Gossen and Bujard (1992) Proc. Natl. Acad. Sci. USA 89:5547, and Paillard (1989) Human Gene Therapy 9:983).

In another embodiment, the recombinant mammalian expression vector is capable of directing expression of the nucleic acid preferentially in a particular cell type (e.g., tissue-specific regulatory elements are used to express the nucleic acid). Non-limiting examples of suitable tissue-specific promoters include the albumin promoter (liver-specific; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) Proc. Natl. Acad. Sci. USA 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, for example, the murine hox promoters (Kessel and Gruss (1990) Science 249:374-379) and the α-fetoprotein promoter (Campes and Tilghman (1989) Genes Dev. 3:537-546).

The invention further provides a recombinant expression vector comprising a DNA molecule of the invention cloned into the expression vector in an antisense orientation. Regulatory sequences (e.g., viral promoters and/or enhancers) operatively linked to a nucleic acid cloned in the antisense orientation can be chosen which direct the constitutive, tissue specific or cell type specific expression of antisense RNA in a variety of cell types. The antisense expression vector can be in the form of a recombinant plasmid, phagemid or attenuated virus.

Another aspect the invention provides a host cell which includes a nucleic acid molecule described herein, e.g., a 47476, 67210, 49875, 46842, 33201, 83378, 84233, 64708, 85041, or 84234 nucleic acid molecule within a recombinant expression vector or a 47476, 67210, 49875, 46842, 33201, 83378, 84233, 64708, 85041, or 84234 nucleic acid molecule containing sequences which allow it to homologously recombine into a specific site of the host cell's genome. The terms “host cell” and “recombinant host cell” are used interchangeably herein. Such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, a 47476, 67210, 49875, 46842, 33201, 83378, 84233, 64708, 85041, or 84234 protein can be expressed in bacterial cells (such as E. coli), insect cells, yeast or mammalian cells (such as Chinese hamster ovary cells (CHO) or COS cells (African green monkey kidney cells CV-1 origin SV40 cells; Gluzman (1981) CellI23:175-182)). Other suitable host cells are known to those skilled in the art.

Vector DNA can be introduced into host cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation.

A host cell of the invention can be used to produce (i.e., express) a 47476, 67210, 49875, 46842, 33201, 83378, 84233, 64708, 85041, or 84234 protein. Accordingly, the invention further provides methods for producing a 47476, 67210, 49875, 46842, 33201, 83378, 84233, 64708, 85041, or 84234 protein using the host cells of the invention. In one embodiment, the method includes culturing the host cell of the invention (into which a recombinant expression vector encoding a 47476, 67210, 49875, 46842, 33201, 83378, 84233, 64708, 85041, or 84234 protein has been introduced) in a suitable medium such that a 47476, 67210, 49875, 46842, 33201, 83378, 84233, 64708, 85041, or 84234 protein is produced. In another embodiment, the method further includes isolating a 47476, 67210, 49875, 46842, 33201, 83378, 84233, 64708, 85041, or 84234 protein from the medium or the host cell.

In another aspect, the invention features, a cell or purified preparation of cells which include a 47476, 67210, 49875, 46842, 33201, 83378, 84233, 64708, 85041, or 84234 transgene, or which otherwise misexpress 47476, 67210, 49875, 46842, 33201, 83378, 84233, 64708, 85041, or 84234. The cell preparation can consist of human or non-human cells, e.g., rodent cells, e.g., mouse or rat cells, rabbit cells, or pig cells. In preferred embodiments, the cell or cells include a 47476, 67210, 49875, 46842, 33201, 83378, 84233, 64708, 85041, or 84234 transgene, e.g., a heterologous form of a 47476, 67210, 49875, 46842, 33201, 83378, 84233, 64708, 85041, or 84234, e.g., a gene derived from humans (in the case of a non-human cell). The 47476, 67210, 49875, 46842, 33201, 83378, 84233, 64708, 85041, or 84234 transgene can be misexpressed, e.g., overexpressed or underexpressed. In other preferred embodiments, the cell or cells include a gene that mis-expresses an endogenous 47476, 67210, 49875, 46842, 33201, 83378, 84233, 64708, 85041, or 84234, e.g., a gene the expression of which is disrupted, e.g., a knockout. Such cells can serve as a model for studying disorders that are related to mutated or mis-expressed 47476, 67210, 49875, 46842, 33201, 83378, 84233, 64708, 85041, or 84234 alleles or for use in drug screening.

In another aspect, the invention features, a human cell, e.g., a hematopoietic, neural, muscle, or hepatic stem cell, transformed with nucleic acid which encodes a subject 47476, 67210, 49875, 46842, 33201, 83378, 84233, 64708, 85041, or 84234 polypeptide.

Also provided are cells, preferably human cells, e.g., human hematopoietic, neural, muscle, hepatic or fibroblast cells, in which an endogenous 47476, 67210, 49875, 46842, 33201, 83378, 84233, 64708, 85041, or 84234 is under the control of a regulatory sequence that does not normally control the expression of the endogenous 47476, 67210, 49875, 46842, 33201, 83378, 84233, 64708, 85041, or 84234 gene. The expression characteristics of an endogenous gene within a cell, e.g., a cell line or microorganism, can be modified by inserting a heterologous DNA regulatory element into the genome of the cell such that the inserted regulatory element is operably linked to the endogenous 47476, 67210, 49875, 46842, 33201, 83378, 84233, 64708, 85041, or 84234 gene. For example, an endogenous 47476, 67210, 49875, 46842, 33201, 83378, 84233, 64708, 85041, or 84234 gene which is “transcriptionally silent,” e.g., not normally expressed, or expressed only at very low levels, may be activated by inserting a regulatory element which is capable of promoting the expression of a normally expressed gene product in that cell. Techniques such as targeted homologous recombinations, can be used to insert the heterologous DNA as described in, e.g., Chappel, U.S. Pat. No. 5,272,071; WO 91/06667, published in May 16, 1991.

In a preferred embodiment, recombinant cells described herein can be used for replacement therapy in a subject. For example, a nucleic acid encoding a 47476, 67210, 49875, 46842, 33201, 83378, 84233, 64708, 85041, or 84234 polypeptide operably linked to an inducible promoter (e.g., a steroid hormone receptor-regulated promoter) is introduced into a human or nonhuman, e.g., mammalian, e.g., porcine recombinant cell. The cell is cultivated and encapsulated in a biocompatible material, such as poly-lysine alginate, and subsequently implanted into the subject. See, e.g., Lanza (1996) Nat. Biotechnol. 14:1107; Joki et al. (2001) Nat. Biotechnol. 19:35; and U.S. Pat. No. 5,876,742. Production of 47476, 67210, 49875, 46842, 33201, 83378, 84233, 64708, 85041, or 84234 polypeptide can be regulated in the subject by administering an agent (e.g., a steroid hormone) to the subject. In another preferred embodiment, the implanted recombinant cells express and secrete an antibody specific for a 47476, 67210, 49875, 46842, 33201, 83378, 84233, 64708, 85041, or 84234 polypeptide. The antibody can be any antibody or any antibody derivative described herein.

47476, 67210, 49875, 46842, 33201, 83378, 84233, 64708, 85041, and 84234 Transgenic Animals

The invention provides non-human transgenic animals. Such animals are useful for studying the function and/or activity of a 47476, 67210, 49875, 46842, 33201, 83378, 84233, 64708, 85041, or 84234 protein and for identifying and/or evaluating modulators of 47476, 67210, 49875, 46842, 33201, 83378, 84233, 64708, 85041, or 84234 activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, and the like. A transgene is exogenous DNA or a rearrangement, e.g., a deletion of endogenous chromosomal DNA, which preferably is integrated into or occurs in the genome of the cells of a transgenic animal. A transgene can direct the expression of an encoded gene product in one or more cell types or tissues of the transgenic animal, other transgenes, e.g., a knockout, reduce expression. Thus, a transgenic animal can be one in which an endogenous 47476, 67210, 49875, 46842, 33201, 83378, 84233, 64708, 85041, or 84234 gene has been altered by, e.g., by homologous recombination between the endogenous gene and an exogenous DNA molecule introduced into a cell of the animal, e.g., an embryonic cell of the animal, prior to development of the animal.

Intronic sequences and polyadenylation signals can also be included in the transgene to increase the efficiency of expression of the transgene. A tissue-specific regulatory sequence(s) can be operably linked to a transgene of the invention to direct expression of a 47476, 67210, 49875, 46842, 33201, 83378, 84233, 64708, 85041, or 84234 protein to particular cells. A transgenic founder animal can be identified based upon the presence of a 47476, 67210, 49875, 46842, 33201, 83378, 84233, 64708, 85041, or 84234 transgene in its genome and/or expression of 47476, 67210, 49875, 46842, 33201, 83378, 84233, 64708, 85041, or 84234 mRNA in tissues or cells of the animals. A transgenic founder animal can then be used to breed additional animals carrying the transgene. Moreover, transgenic animals carrying a transgene encoding a 47476, 67210, 49875, 46842, 33201, 83378, 84233, 64708, 85041, or 84234 protein can further be bred to other transgenic animals carrying other transgenes.

47476, 67210, 49875, 46842, 33201, 83378, 84233, 64708, 85041, or 84234 proteins or polypeptides can be expressed in transgenic animals or plants, e.g., a nucleic acid encoding the protein or polypeptide can be introduced into the genome of an animal. In preferred embodiments the nucleic acid is placed under the control of a tissue specific promoter, e.g., a milk or egg specific promoter, and recovered from the milk or eggs produced by the animal. Suitable animals are mice, pigs, cows, goats, and sheep.

The invention also includes a population of cells from a transgenic animal, as discussed, e.g., below.

Uses of 47476, 67210, 49875, 46842, 33201, 83378, 84233, 64708, 85041, and 84234

The nucleic acid molecules, proteins, protein homologues, and antibodies described herein can be used in one or more of the following methods: a) screening assays; b) predictive medicine (e.g., diagnostic assays, prognostic assays, monitoring clinical trials, and pharmacogenetics); and c) methods of treatment (e.g., therapeutic and prophylactic).

The isolated nucleic acid molecules of the invention can be used, for example, to express a 47476, 67210, 49875, 46842, 33201, 83378, 84233, 64708, 85041, or 84234 protein (e.g., via a recombinant expression vector in a host cell in gene therapy applications), to detect a 47476, 67210, 49875, 46842, 33201, 83378, 84233, 64708, 85041, or 84234 mRNA (e.g., in a biological sample) or a genetic alteration in a 47476, 67210, 49875, 46842, 33201, 83378, 84233, 64708, 85041, or 84234 gene, and to modulate 47476, 67210, 49875, 46842, 33201, 83378, 84233, 64708, 85041, or 84234 activity, as described further below. The 47476, 67210, 49875, 46842, 33201, 83378, 84233, 64708, 85041, or 84234 proteins can be used to treat disorders characterized by insufficient or excessive production of a 47476, 67210, 49875, 46842, 33201, 83378, 84233, 64708, 85041, or 84234 substrate or production of 47476, 67210, 49875, 46842, 33201, 83378, 84233, 64708, 85041, or 84234 inhibitors. In addition, the 47476, 67210, 49875, 46842, 33201, 83378, 84233, 64708, 85041, or 84234 proteins can be used to screen for naturally occurring 47476, 67210, 49875, 46842, 33201, 83378, 84233, 64708, 85041, or 84234 substrates, to screen for drugs or compounds which modulate 47476, 67210, 49875, 46842, 33201, 83378, 84233, 64708, 85041, or 84234 activity, as well as to treat disorders characterized by insufficient or excessive production of 47476, 67210, 49875, 46842, 33201, 83378, 84233, 64708, 85041, or 84234 protein or production of 47476, 67210, 49875, 46842, 33201, 83378, 84233, 64708, 85041, or 84234 protein forms which have decreased, aberrant or unwanted activity compared to 47476, 67210, 49875, 46842, 33201, 83378, 84233, 64708, 85041, or 84234 wild type protein (e.g., immunological disorders, neurological disorders, metabolic disorders, cellular proliferation and/or differentiation disorders, disorders of metal ion imbalance, protein trafficing disorders, or cardiovascular disorders). Moreover, the anti-47476, 67210, 49875, 46842, 33201, 83378, 84233, 64708, 85041, or 84234 antibodies of the invention can be used to detect and isolate 47476, 67210, 49875, 46842, 33201, 83378, 84233, 64708, 85041, or 84234 proteins, regulate the bioavailability of 47476, 67210, 49875, 46842, 33201, 83378, 84233, 64708, 85041, or 84234 proteins, and modulate 47476, 67210, 49875, 46842, 33201, 83378, 84233, 64708, 85041, or 84234 activity.

A method of evaluating a compound for the ability to interact with, e.g., bind, a subject 47476, 67210, 49875, 46842, 33201, 83378, 84233, 64708, 85041, or 84234 polypeptide is provided. The method includes: contacting the compound with the subject 47476, 67210, 49875, 46842, 33201, 83378, 84233, 64708, 85041, or 84234 polypeptide; and evaluating ability of the compound to interact with, e.g., to bind or form a complex with the subject 47476, 67210, 49875, 46842, 33201, 83378, 84233, 64708, 85041, or 84234 polypeptide. This method can be performed in vitro, e.g., in a cell free system, or in vivo, e.g., in a two-hybrid interaction trap assay. This method can be used to identify naturally occurring molecules that interact with subject 47476, 67210, 49875, 46842, 33201, 83378, 84233, 64708, 85041, or 84234 polypeptide. It can also be used to find natural or synthetic inhibitors of subject 47476, 67210, 49875, 46842, 33201, 83378, 84233, 64708, 85041, or 84234 polypeptide. Screening methods are discussed in more detail below.

47476, 67210, 49875, 46842, 33201, 83378, 84233, 64708, 85041, and 84234 Screening Assays

The invention provides methods (also referred to herein as “screening assays”) for identifying modulators, i.e., candidate or test compounds or agents (e.g., proteins, peptides, peptidomimetics, peptoids, small molecules or other drugs) which bind to 47476, 67210, 49875, 46842, 33201, 83378, 84233, 64708, 85041, or 84234 proteins, have a stimulatory or inhibitory effect on, for example, 47476, 67210, 49875, 46842, 33201, 83378, 84233, 64708, 85041, or 84234 expression or 47476, 67210, 49875, 46842, 33201, 83378, 84233, 64708, 85041, or 84234 activity, or have a stimulatory or inhibitory effect on, for example, the expression or activity of a 47476, 67210, 49875, 46842, 33201, 83378, 84233, 64708, 85041, or 84234-interacting molecule, e.g., substrate molecule. Compounds thus identified can be used to modulate the activity of target gene products (e.g., 47476, 67210, 49875, 46842, 33201, 83378, 84233, 64708, 85041, or 84234 genes) in a therapeutic protocol, to elaborate the biological function of the target gene product, or to identify compounds that disrupt normal target gene interactions.

In one embodiment, the invention provides assays for screening candidate or test compounds which are substrates of a 47476, 67210, 49875, 46842, 33201, 83378, 84233, 64708, 85041, or 84234 protein or polypeptide or a biologically active portion thereof. In another embodiment, the invention provides assays for screening candidate or test compounds that bind to or modulate an activity of a 47476, 67210, 49875, 46842, 33201, 83378, 84233, 64708, 85041, or 84234 protein or polypeptide or a biologically active portion thereof.

In one embodiment, an activity of a 47476 protein can be assayed as follows: (a) introduce a 47476 necleic acid expression construct into a cell such that 47476 protein is produced; (b) activate a signal transduction pathway that utilizes a ras superfamily member that can be activated by a 47476 protein; and (c) evaluate the ability the 47476 protein or functional fragment thereof to modulate the activated signal transduction pathway, as compared to a control cell that lacks the 47476 expression construct. Assays for determining the activity level of a signal transduction pathway will depend on the particular signal transduction pathway. Nevertheless, such assays are known in the art and include, e.g., detection of GTP loading of ras or ras-like proteins, gel electrophoresis of downstream targets, e.g., MAP kinase, or detection of the level of phosphorylation of various components of the signaling pathway.

In one embodiment, an activity of a 67210 protein can be assayed as follows: (a) contacting an acceptor molecule (e.g., a lipid, protein, or carbohydrate) with a 67210 protein or functional fragment thereof in the presence of an activated mono- or oligosaccharide residue; and (b) evaluating the ability the 67210 protein or functional fragment thereof to initiate or elongate the carbohydrate chain. Assays for determining whether a carbohydrate modification has been made are known in the art and include, e.g., gel electrophoresis or antibody affinity.

In one embodiment, an activity of a 49875 protein can be assayed as follows. (a) contact a nucleic acid, e.g., a nucleic acid duplex, with a 49875 protein or functional fragment thereof in the presence of an NTP, e.g., GTP or ATP, and (b) evaluate the ability of the 49875 protein or functional fragment thereof to cause the unwinding of the nucleic acid. Assays for determining if a nucleic acid is wound or unwound are known in the art and include, e.g., gel electrophoresis.

In one embodiment, an activity of a 46842 protein can be assayed as described in Kam et al. (2000), J Biol Chem 275:9653, or Dowler et al. (2000), Biochem J 351:19, the contents of which are incorporated herein by reference.

In one embodiment, an activity of a 33201 protein can be assayed as follows: (a) contacting a cell that expresses a 33201 protein, or a fragment thereof, with a known substrate, e.g., an alcohol or quinone substrate; and (b) evaluate the ability the 33201 protein, or functional fragment thereof, to oxidize or reduce the substrate. Assays for determining whether a substrate has been oxidized or reduced are known in the art.

In one embodiment, an activity of a 83378, 84233, 64708, 85041, or 84234 protein can be assayed by measuring ion transport or by visualizing ions contained within a cell or a cellular compartment, e.g., in the presence and/or absence of a 83378, 84233, 64708, 85041, or 84234 protein. For example methods of detecting zinc transport or visualizing zinc within cellular compartments, e.g., using a zinc-specific fluorescent probe, are described in detail in, e.g., Palmiter et al. (1995) EMBO J. 14:639-649, Palmiter et al. (1996) EMBO J. 15:1784-1791, and Palmiter et al. (1996) Proc. Natl. Acad. Sci. USA 93:14934-14939, the contents of which are incorporated herein by reference.

The test compounds of the present invention can be obtained using any of the numerous approaches in combinatorial library methods known in the art, including: biological libraries; peptoid libraries (libraries of molecules having the functionalities of peptides, but with a novel, non-peptide backbone which are resistant to enzymatic degradation but which nevertheless remain bioactive; see, e.g., Zuckermann, R. N. et al. (1994) J. Med. Chem. 37:2678-85); spatially addressable parallel solid phase or solution phase libraries; synthetic library methods requiring deconvolution; the ‘one-bead one-compound’ library method; and synthetic library methods using affinity chromatography selection. The biological library and peptoid library approaches are limited to peptide libraries, while the other four approaches are applicable to peptide, non-peptide oligomer or small molecule libraries of compounds (Lam (1997) Anticancer Drug Des. 12:145).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and Gallop et al. (1994) J. Med. Chem. 37:1233.

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner, U.S. Pat. No. 5,223,409), spores (Ladner U.S. Pat. No. 5,223,409), plasmids (Cull et al. (1992) Proc Natl Acad Sci USA 89:1865-1869) or on phage (Scott and Smith (1990) Science 249:386-390; Devlin (1990) Science 249:404-406; Cwirla et al. (1990) Proc. Natl. Acad. Sci. 87:6378-6382; Felici (1991) J. Mol. Biol. 222:301-310; Ladner supra.).

In one embodiment, an assay is a cell-based assay in which a cell which expresses a 47476 protein or biologically active portion thereof is contacted with a test compound, and the ability of the test compound to modulate 47476 activity is determined. Determining the ability of the test compound to modulate 47476 activity can be accomplished, e.g., by monitoring the ability of 47476 polypeptide or proteins to activate one or more ras superfamily proteins, e.g., as measured by cellular characteristics at least partially controlled by ras superfamily members, e.g., cell shape, motility, growth, adhesion, or differentiation. The cell, for example, can be of mammalian origin, e.g., human.

In another embodiment, an assay is a cell-based assay in which a cell which expresses a 67210 protein or biologically active portion thereof is contacted with a test compound, and the ability of the test compound to modulate 67210 activity is determined. Determining the ability of the test compound to modulate 67210 activity can be accomplished, e.g., by monitoring the ability of 67210 add sugar residues to an appropriate substrate, e.g., as measured by the appearance of the modified substrate molecule of the cell surface or by changes in signal transduction with in the cell that result in changes in cellular behavior, e.g., changes in cell shape, motility, growth, adhesion, or differentiation. The cell, for example, can be of mammalian origin, e.g., human.

In another embodiment, an assay is a cell-based assay in which a cell which expresses a 49875 protein or biologically active portion thereof is contacted with a test compound, and the ability of the test compound to modulate 49875 activity is determined. Determining the ability of the test compound to modulate 49875 activity can be accomplished, e.g., by monitoring the ability of 49875 molecules to unwind duplex nucleic acid molecules, e.g., as measured by changes in transcription, e.g., using a GFP construct and GFP expression as a readout, or changes in cellular behavior, e.g., changes in cell shape, motility, growth, adhesion, or differentiation. The cell, for example, can be of mammalian origin, e.g., human.

In another embodiment, an assay is a cell-based assay in which a cell which expresses a 46842 protein or biologically active portion thereof is contacted with a test compound, and the ability of the test compound to modulate 46842 activity is determined. Determining the ability of the test compound to modulate 46842 activity can be accomplished, e.g., by monitoring the ability of 46842 molecules to stimulate the GTPase activity of an Arf or arf-like protein, e.g., as measured by changes in cell shape or protein trafficking, e.g., using an appropriate marker protein that is transported through the secretory pathway. The cell, for example, can be of mammalian origin, e.g., human.

In another embodiment, an assay is a cell-based assay in which a cell which expresses a 83378, 84233, 64708, 85041, or 84234 protein or biologically active portion thereof is contacted with a test compound, and the ability of the test compound to modulate 83378, 84233, 64708, 85041, or 84234 activity is determined. Determining the ability of the test compound to modulate 46842 activity can be accomplished, e.g., by monitoring the ability of 83378, 84233, 64708, 85041, or 84234 molecules to transport metal ions, e.g., as measured by changes in the subcellular location of metal ions, e.g., zinc ions, or in the cellular response to the presence of metal ions, e.g., programmed cell death. The cell, for example, can be of mammalian origin, e.g., human.

The ability of the test compound to modulate 47476, 67210, 49875, 46842, 33201, 83378, 84233, 64708, 85041, or 84234 binding to a compound, e.g., a 47476, 67210, 49875, 46842, 33201, 83378, 84233, 64708, 85041, or 84234 substrate, or to bind to 47476, 67210, 49875, 46842, 33201, 83378, 84233, 64708, 85041, or 84234 can also be evaluated. This can be accomplished, for example, by coupling the compound, e.g., the substrate, with a radioisotope or enzymatic label such that binding of the compound, e.g., the substrate, to 47476, 67210, 49875, 46842, 33201, 83378, 84233, 64708, 85041, or 84234 can be determined by detecting the labeled compound, e.g., substrate, in a complex. Alternatively, 47476, 67210, 49875, 46842, 33201, 83378, 84233, 64708, 85041, or 84234 could be coupled with a radioisotope or enzymatic label to monitor the ability of a test compound to modulate 47476, 67210, 49875, 46842, 33201, 83378, 84233, 64708, 85041, or 84234 binding to a 47476, 67210, 49875, 46842, 33201, 83378, 84233, 64708, 85041, or 84234 substrate in a complex. For example, compounds (e.g., 47476, 67210, 49875, 46842, 33201, 83378, 84233, 64708, 85041, or 84234 substrates) can be labeled with ¹²⁵I, ³⁵S, ¹⁴C, or ³H, either directly or indirectly, and the radioisotope detected by direct counting of radioemmission or by scintillation counting. Alternatively, compounds can be enzymatically labeled with, for example, horseradish peroxidase, alkaline phosphatase, or luciferase, and the enzymatic label detected by determination of conversion of an appropriate substrate to product.

The ability of a compound (e.g., a 47476, 67210, 49875, 46842, 33201, 83378, 84233, 64708, 85041, or 84234 substrate) to interact with 47476, 67210, 49875, 46842, 33201, 83378, 84233, 64708, 85041, or 84234 with or without the labeling of any of the interactants can be evaluated. For example, a microphysiometer can be used to detect the interaction of a compound with 47476, 67210, 49875, 46842, 33201, 83378, 84233, 64708, 85041, or 84234 without the labeling of either the compound or the 47476, 67210, 49875, 46842, 33201, 83378, 84233, 64708, 85041, or 84234. McConnell, H. M. et al. (1992) Science 257:1906-1912. As used herein, a “microphysiometer” (e.g., Cytosensor) is an analytical instrument that measures the rate at which a cell acidifies its environment using a light-addressable potentiometric sensor (LAPS). Changes in this acidification rate can be used as an indicator of the interaction between a compound and 47476, 67210, 49875, 46842, 33201, 83378, 84233, 64708, 85041, or 84234.

In yet another embodiment, a cell-free assay is provided in which a 47476, 67210, 49875, 46842, 33201, 83378, 84233, 64708, 85041, or 84234 protein or biologically active portion thereof is contacted with a test compound and the ability of the test compound to bind to the 47476, 67210, 49875, 46842, 33201, 83378, 84233, 64708, 85041, or 84234 protein or biologically active portion thereof is evaluated. Preferred biologically active portions of the 47476, 67210, 49875, 46842, 33201, 83378, 84233, 64708, 85041, or 84234 proteins to be used in assays of the present invention include fragments which participate in interactions with non-47476, 67210, 49875, 46842, 33201, 83378, 84233, 64708, 85041, or 84234 molecules, e.g., fragments with high surface probability scores.

Soluble and/or membrane-bound forms of isolated proteins (e.g., 47476, 67210, 49875, 46842, 33201, 83378, 84233, 64708, 85041, or 84234 proteins or biologically active portions thereof) can be used in the cell-free assays of the invention. When membrane-bound forms of the protein are used, it may be desirable to utilize a solubilizing agent. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)ₙ, 3-[(3-cholamidopropyl)dimethylamminio]-1-propane sulfonate (CHAPS), 3-[(3-cholamidopropyl)dimethylamminio]-2-hydroxy-1-propane sulfonate (CHAPSO), or N-dodecyl=N,N-dimethyl-3-ammonio-1-propane sulfonate.

Cell-free assays involve preparing a reaction mixture of the target gene protein and the test compound under conditions and for a time sufficient to allow the two components to interact and bind, thus forming a complex that can be removed and/or detected.

The interaction between two molecules can also be detected, e.g., using fluorescence energy transfer (FET) (see, for example, Lakowicz et al., U.S. Pat. No. 5,631,169; Stavrianopoulos, et al., U.S. Pat. No. 4,868,103). A fluorophore label on the first, ‘donor’ molecule is selected such that its emitted fluorescent energy will be absorbed by a fluorescent label on a second, ‘acceptor’ molecule, which in turn is able to fluoresce due to the absorbed energy. Alternately, the ‘donor’ protein molecule may simply utilize the natural fluorescent energy of tryptophan residues. Labels are chosen that emit different wavelengths of light, such that the ‘acceptor’ molecule label may be differentiated from that of the ‘donor’. Since the efficiency of energy transfer between the labels is related to the distance separating the molecules, the spatial relationship between the molecules can be assessed. In a situation in which binding occurs between the molecules, the fluorescent emission of the ‘acceptor’ molecule label in the assay should be maximal. An FET binding event can be conveniently measured through standard fluorometric detection means well known in the art (e.g., using a fluorimeter).

In another embodiment, determining the ability of the 47476, 67210, 49875, 46842, 33201, 83378, 84233, 64708, 85041, or 84234 protein to bind to a target molecule can be accomplished using real-time Biomolecular Interaction Analysis (BIA) (see, e.g., Sjolander, S. and Urbaniczky, C. (1991) Anal. Chem. 63:2338-2345 and Szabo et al. (1995) Curr. Opin. Struct. Biol. 5:699-705). “Surface plasmon resonance” or “BIA” detects biospecific interactions in real time, without labeling any of the interactants (e.g., BIAcore). Changes in the mass at the binding surface (indicative of a binding event) result in alterations of the refractive index of light near the surface (the optical phenomenon of surface plasmon resonance (SPR)), resulting in a detectable signal which can be used as an indication of real-time reactions between biological molecules.

In one embodiment, the target gene product or the test substance is anchored onto a solid phase. The target gene product/test compound complexes anchored on the solid phase can be detected at the end of the reaction. Preferably, the target gene product can be anchored onto a solid surface, and the test compound, (which is not anchored), can be labeled, either directly or indirectly, with detectable labels discussed herein.

It may be desirable to immobilize either 47476, 67210, 49875, 46842, 33201, 83378, 84233, 64708, 85041, or 84234, an anti-47476, 67210, 49875, 46842, 33201, 83378, 84233, 64708, 85041, or 84234 antibody or its target molecule to facilitate separation of complexed from uncomplexed forms of one or both of the proteins, as well as to accommodate automation of the assay. Binding of a test compound to a 47476, 67210, 49875, 46842, 33201, 83378, 84233, 64708, 85041, or 84234 protein, or interaction of a 47476, 67210, 49875, 46842, 33201, 83378, 84233, 64708, 85041, or 84234 protein with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided which adds a domain that allows one or both of the proteins to be bound to a matrix. For example, glutathione-S-transferase/47476, 67210, 49875, 46842, 33201, 83378, 84233, 64708, 85041, or 84234 fusion proteins or glutathione-S-transferase/target fusion proteins can be adsorbed onto glutathione sepharose beads (Sigma Chemical, St. Louis, Mo.) or glutathione derivatized microtiter plates, which are then combined with the test compound or the test compound and either the non-adsorbed target protein or 47476, 67210, 49875, 46842, 33201, 83378, 84233, 64708, 85041, or 84234 protein, and the mixture incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described above. Alternatively, the complexes can be dissociated from the matrix, and the level of 47476, 67210, 49875, 46842, 33201, 83378, 84233, 64708, 85041, or 84234 binding or activity determined using standard techniques.

Other techniques for immobilizing either a 47476, 67210, 49875, 46842, 33201, 83378, 84233, 64708, 85041, or 84234 protein or a target molecule on matrices include using conjugation of biotin and streptavidin. Biotinylated 47476, 67210, 49875, 46842, 33201, 83378, 84233, 64708, 85041, or 84234 protein or target molecules can be prepared from biotin-NHS(N-hydroxy-succinimide) using techniques known in the art (e.g., biotinylation kit, Pierce Chemicals, Rockford, Ill.), and immobilized in the wells of streptavidin-coated 96 well plates (Pierce Chemical).

In order to conduct the assay, the non-immobilized component is added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways. Where the previously non-immobilized component is pre-labeled, the detection of label immobilized on the surface indicates that complexes were formed. Where the previously non-immobilized component is not pre-labeled, an indirect label can be used to detect complexes anchored on the surface; e.g., using a labeled antibody specific for the immobilized component (the antibody, in turn, can be directly labeled or indirectly labeled with, e.g., a labeled anti-Ig antibody).

In one embodiment, this assay is performed utilizing antibodies reactive with 47476, 67210, 49875, 46842, 33201, 83378, 84233, 64708, 85041, or 84234 protein or target molecules but which do not interfere with binding of the 47476, 67210, 49875, 46842, 33201, 83378, 84233, 64708, 85041, or 84234 protein to its target molecule. Such antibodies can be derivatized to the wells of the plate, and unbound target or 47476, 67210, 49875, 46842, 33201, 83378, 84233, 64708, 85041, or 84234 protein trapped in the wells by antibody conjugation. Methods for detecting such complexes, in addition to those described above for the GST-immobilized complexes, include immunodetection of complexes using antibodies reactive with the 47476, 67210, 49875, 46842, 33201, 83378, 84233, 64708, 85041, or 84234 protein or target molecule, as well as enzyme-linked assays which rely on detecting an enzymatic activity associated with the 47476, 67210, 49875, 46842, 33201, 83378, 84233, 64708, 85041, or 84234 protein or target molecule.

Alternatively, cell free assays can be conducted in a liquid phase. In such an assay, the reaction products are separated from unreacted components, by any of a number of standard techniques, including but not limited to: differential centrifugation (see, for example, Rivas, G., and Minton, A. P., (1993) Trends Biochem Sci 18:284-7); chromatography (gel filtration chromatography, ion-exchange chromatography); electrophoresis (see, e.g., Ausubel, F. et al., eds. Current Protocols in Molecular Biology 1999, J. Wiley: New York.); and immunoprecipitation (see, for example, Ausubel, F. et al., eds. (1999) Current Protocols in Molecular Biology, J. Wiley: New York). Such resins and chromatographic techniques are known to one skilled in the art (see, e.g., Heegaard, N. H., (1998) J Mol Recognit 11:141-8; Hage, D. S., and Tweed, S. A. (1997) J Chromatogr B Biomed Sci Appl. 699:499-525). Further, fluorescence energy transfer may also be conveniently utilized, as described herein, to detect binding without further purification of the complex from solution.

In a preferred embodiment, the assay includes contacting the 47476, 67210, 49875, 46842, 33201, 83378, 84233, 64708, 85041, or 84234 protein or biologically active portion thereof with a known compound which binds 47476, 67210, 49875, 46842, 33201, 83378, 84233, 64708, 85041, or 84234 to form an assay mixture, contacting the assay mixture with a test compound, and determining the ability of the test compound to interact with a 47476, 67210, 49875, 46842, 33201, 83378, 84233, 64708, 85041, or 84234 protein, wherein determining the ability of the test compound to interact with a 47476, 67210, 49875, 46842, 33201, 83378, 84233, 64708, 85041, or 84234 protein includes determining the ability of the test compound to preferentially bind to 47476, 67210, 49875, 46842, 33201, 83378, 84233, 64708, 85041, or 84234 or biologically active portion thereof, or to modulate the activity of a target molecule, as compared to the known compound.

The target gene products of the invention can, in vivo, interact with one or more cellular or extracellular macromolecules, such as proteins. For the purposes of this discussion, such cellular and extracellular macromolecules are referred to herein as “binding partners.” Compounds that disrupt such interactions can be useful in regulating the activity of the target gene product. Such compounds can include, but are not limited to molecules such as antibodies, peptides, and small molecules. The preferred target genes/products for use in this embodiment are the 47476, 67210, 49875, 46842, 33201, 83378, 84233, 64708, 85041, or 84234 genes herein identified. In an alternative embodiment, the invention provides methods for determining the ability of the test compound to modulate the activity of a 47476, 67210, 49875, 46842, 33201, 83378, 84233, 64708, 85041, or 84234 protein through modulation of the activity of a downstream effector of a 47476, 67210, 49875, 46842, 33201, 83378, 84233, 64708, 85041, or 84234 target molecule. For example, the activity of the effector molecule on an appropriate target can be determined, or the binding of the effector to an appropriate target can be determined, as previously described.

To identify compounds that interfere with the interaction between the target gene product and its cellular or extracellular binding partner(s), a reaction mixture containing the target gene product and the binding partner is prepared, under conditions and for a time sufficient, to allow the two products to form complex. In order to test an inhibitory agent, the reaction mixture is provided in the presence and absence of the test compound. The test compound can be initially included in the reaction mixture, or can be added at a time subsequent to the addition of the target gene and its cellular or extracellular binding partner. Control reaction mixtures are incubated without the test compound or with a placebo. The formation of any complexes between the target gene product and the cellular or extracellular binding partner is then detected. The formation of a complex in the control reaction, but not in the reaction mixture containing the test compound, indicates that the compound interferes with the interaction of the target gene product and the interactive binding partner. Additionally, complex formation within reaction mixtures containing the test compound and normal target gene product can also be compared to complex formation within reaction mixtures containing the test compound and mutant target gene product. This comparison can be important in those cases wherein it is desirable to identify compounds that disrupt interactions of mutant but not normal target gene products.

These assays can be conducted in a heterogeneous or homogeneous format. Heterogeneous assays involve anchoring either the target gene product or the binding partner onto a solid phase, and detecting complexes anchored on the solid phase at the end of the reaction. In homogeneous assays, the entire reaction is carried out in a liquid phase. In either approach, the order of addition of reactants can be varied to obtain different information about the compounds being tested. For example, test compounds that interfere with the interaction between the target gene products and the binding partners, e.g., by competition, can be identified by conducting the reaction in the presence of the test substance. Alternatively, test compounds that disrupt preformed complexes, e.g., compounds with higher binding constants that displace one of the components from the complex, can be tested by adding the test compound to the reaction mixture after complexes have been formed. The various formats are briefly described below.

In a heterogeneous assay system, either the target gene product or the interactive cellular or extracellular binding partner, is anchored onto a solid surface (e.g., a microtiter plate), while the non-anchored species is labeled, either directly or indirectly. The anchored species can be immobilized by non-covalent or covalent attachments. Alternatively, an immobilized antibody specific for the species to be anchored can be used to anchor the species to the solid surface.

In order to conduct the assay, the partner of the immobilized species is exposed to the coated surface with or without the test compound. After the reaction is complete, unreacted components are removed (e.g., by washing) and any complexes formed will remain immobilized on the solid surface. Where the non-immobilized species is pre-labeled, the detection of label immobilized on the surface indicates that complexes were formed. Where the non-immobilized species is not pre-labeled, an indirect label can be used to detect complexes anchored on the surface; e.g., using a labeled antibody specific for the initially non-immobilized species (the antibody, in turn, can be directly labeled or indirectly labeled with, e.g., a labeled anti-Ig antibody). Depending upon the order of addition of reaction components, test compounds that inhibit complex formation or that disrupt preformed complexes can be detected.

Alternatively, the reaction can be conducted in a liquid phase in the presence or absence of the test compound, the reaction products separated from unreacted components, and complexes detected; e.g., using an immobilized antibody specific for one of the binding components to anchor any complexes formed in solution, and a labeled antibody specific for the other partner to detect anchored complexes. Again, depending upon the order of addition of reactants to the liquid phase, test compounds that inhibit complex or that disrupt preformed complexes can be identified.

In an alternate embodiment of the invention, a homogeneous assay can be used. For example, a preformed complex of the target gene product and the interactive cellular or extracellular binding partner product is prepared in that either the target gene products or their binding partners are labeled, but the signal generated by the label is quenched due to complex formation (see, e.g., U.S. Pat. No. 4,109,496 that utilizes this approach for immunoassays). The addition of a test substance that competes with and displaces one of the species from the preformed complex will result in the generation of a signal above background. In this way, test substances that disrupt target gene product-binding partner interaction can be identified.

In yet another aspect, the 47476, 67210, 49875, 46842, 33201, 83378, 84233, 64708, 85041, or 84234 proteins can be used as “bait proteins” in a two-hybrid assay or three-hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Biotechniques 14:920-924; Iwabuchi et al. (1993) Oncogene 8:1693-1696; and Brent WO94/10300), to identify other proteins, which bind to or interact with 47476, 67210, 49875, 46842, 33201, 83378, 84233, 64708, 85041, or 84234 (“47476, 67210, 49875, 46842, 33201, 83378, 84233, 64708, 85041, or 84234-binding proteins” or “47476, 67210, 49875, 46842, 33201, 83378, 84233, 64708, 85041, or 84234-bp”) and are involved in 47476, 67210, 49875, 46842, 33201, 83378, 84233, 64708, 85041, or 84234 activity. Such 47476, 67210, 49875, 46842, 33201, 83378, 84233, 64708, 85041, or 84234-bps can be activators or inhibitors of signals by the 47476, 67210, 49875, 46842, 33201, 83378, 84233, 64708, 85041, or 84234 proteins or 47476, 67210, 49875, 46842, 33201, 83378, 84233, 64708, 85041, or 84234 targets as, for example, downstream elements of a 47476, 67210, 49875, 46842, 33201, 83378, 84233, 64708, 85041, or 84234-mediated signaling pathway.

The two-hybrid system is based on the modular nature of most transcription factors, which consist of separable DNA-binding and activation domains. Briefly, the assay utilizes two different DNA constructs. In one construct, the gene that codes for a 47476, 67210, 49875, 46842, 33201, 83378, 84233, 64708, 85041, or 84234 protein is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. (Alternatively the: 47476, 67210, 49875, 46842, 33201, 83378, 84233, 64708, 85041, or 84234 protein can be the fused to the activator domain.) If the “bait” and the “prey” proteins are able to interact, in vivo, forming a 47476, 67210, 49875, 46842, 33201, 83378, 84233, 64708, 85041, or 84234-dependent complex, the DNA-binding and activation domains of the transcription factor are brought into close proximity. This proximity allows transcription of a reporter gene (e.g., lacZ) which is operably linked to a transcriptional regulatory site responsive to the transcription factor. Expression of the reporter gene can be detected and cell colonies containing the functional transcription factor can be isolated and used to obtain the cloned gene which encodes the protein which interacts with the 47476, 67210, 49875, 46842, 33201, 83378, 84233, 64708, 85041, or 84234 protein.

In another embodiment, modulators of 47476, 67210, 49875, 46842, 33201, 83378, 84233, 64708, 85041, or 84234 expression are identified. For example, a cell or cell free mixture is contacted with a candidate compound and the expression of 47476, 67210, 49875, 46842, 33201, 83378, 84233, 64708, 85041, or 84234 mRNA or protein evaluated relative to the level of expression of 47476, 67210, 49875, 46842, 33201, 83378, 84233, 64708, 85041, or 84234 mRNA or protein in the absence of the candidate compound. When expression of 47476, 67210, 49875, 46842, 33201, 83378, 84233, 64708, 85041, or 84234 mRNA or protein is greater in the presence of the candidate compound than in its absence, the candidate compound is identified as a stimulator of 47476, 67210, 49875, 46842, 33201, 83378, 84233, 64708, 85041, or 84234 mRNA or protein expression. Alternatively, when expression of 47476, 67210, 49875, 46842, 33201, 83378, 84233, 64708, 85041, or 84234 mRNA or protein is less (statistically significantly less) in the presence of the candidate compound than in its absence, the candidate compound is identified as an inhibitor of 47476, 67210, 49875, 46842, 33201, 83378, 84233, 64708, 85041, or 84234 mRNA or protein expression. The level of 47476, 67210, 49875, 46842, 33201, 83378, 84233, 64708, 85041, or 84234 mRNA or protein expression can be determined by methods described herein for detecting 47476, 67210, 49875, 46842, 33201, 83378, 84233, 64708, 85041, or 84234 mRNA or protein.

In another aspect, the invention pertains to a combination of two or more of the assays described herein. For example, a modulating agent can be identified using a cell-based or a cell free assay, and the ability of the agent to modulate the activity of a 47476, 67210, 49875, 46842, 33201, 83378, 84233, 64708, 85041, or 84234 protein can be confirmed in vivo, e.g., in an animal such as an animal model for immunological disorders, neurological disorders, metabolic disorders, cellular proliferation and/or differentiation disorders, disorders of metal ion imbalance, protein trafficing disorders, or cardiovascular disorders.

This invention further pertains to novel agents identified by the above-described screening assays. Accordingly, it is within the scope of this invention to further use an agent identified as described herein (e.g., a 47476, 67210, 49875, 46842, 33201, 83378, 84233, 64708, 85041, or 84234 modulating agent, an antisense 47476, 67210, 49875, 46842, 33201, 83378, 84233, 64708, 85041, or 84234 nucleic acid molecule, a 47476, 67210, 49875, 46842, 33201, 83378, 84233, 64708, 85041, or 84234-specific antibody, or a 47476, 67210, 49875, 46842, 33201, 83378, 84233, 64708, 85041, or 84234-binding partner) in an appropriate animal model to determine the efficacy, toxicity, side effects, or mechanism of action, of treatment with such an agent. Furthermore, novel agents identified by the above-described screening assays can be used for treatments as described herein.

47476, 67210, 49875, 46842, 33201, 83378, 84233, 64708, 85041, and 84234 Detection Assays

Portions or fragments of the nucleic acid sequences identified herein can be used as polynucleotide reagents. For example, these sequences can be used to: (i) map their respective genes on a chromosome e.g., to locate gene regions associated with genetic disease or to associate 47476, 67210, 49875, 46842, 33201, 83378, 84233, 64708, 85041, or 84234 with a disease; (ii) identify an individual from a minute biological sample (tissue typing); and (iii) aid in forensic identification of a biological sample. These applications are described in the subsections below.

47476, 67210, 49875, 46842, 33201, 83378, 84233, 64708, 85041, and 84234 Chromosome Mapping

The 47476, 67210, 49875, 46842, 33201, 83378, 84233, 64708, 85041, or 84234 nucleotide sequences or portions thereof can be used to map the location of the 47476, 67210, 49875, 46842, 33201, 83378, 84233, 64708, 85041, or 84234 genes on a chromosome. This process is called chromosome mapping. Chromosome mapping is useful in correlating the 47476, 67210, 49875, 46842, 33201, 83378, 84233, 64708, 85041, or 84234 sequences with genes associated with disease.

Briefly, 47476, 67210, 49875, 46842, 33201, 83378, 84233, 64708, 85041, or 84234 genes can be mapped to chromosomes by preparing PCR primers (preferably 15-25 bp in length) from the 47476, 67210, 49875, 46842, 33201, 83378, 84233, 64708, 85041, or 84234 nucleotide sequences. These primers can then be used for PCR screening of somatic cell hybrids containing individual human chromosomes. Only those hybrids containing the human gene corresponding to the 47476, 67210, 49875, 46842, 33201, 83378, 84233, 64708, 85041, or 84234 sequences will yield an amplified fragment.

A panel of somatic cell hybrids in which each cell line contains either a single human chromosome or a small number of human chromosomes, and a full set of mouse chromosomes, can allow easy mapping of individual genes to specific human chromosomes. (D'Eustachio P. et al. (1983) Science 220:919-924).

Other mapping strategies e.g., in situ hybridization (described in Fan, Y. et al. (1990) Proc. Natl. Acad. Sci. USA, 87:6223-27), pre-screening with labeled flow-sorted chromosomes, and pre-selection by hybridization to chromosome specific cDNA libraries can be used to map 47476, 67210, 49875, 46842, 33201, 83378, 84233, 64708, 85041, or 84234 to a chromosomal location.

Fluorescence in situ hybridization (FISH) of a DNA sequence to a metaphase chromosomal spread can further be used to provide a precise chromosomal location in one step. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases will suffice to get good results at a reasonable amount of time. For a review of this technique, see Verma et al., Human Chromosomes: A Manual of Basic Techniques ((1988) Pergamon Press, New York).

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. (Such data are found, for example, in V. McKusick, Mendelian Inheritance in Man, available on-line through Johns Hopkins University Welch Medical Library). The relationship between a gene and a disease, mapped to the same chromosomal region, can then be identified through linkage analysis (co-inheritance of physically adjacent genes), described in, for example, Egeland, J. et al. (1987) Nature, 325:783-787.

Moreover, differences in the DNA sequences between individuals affected and unaffected with a disease associated with the 47476, 67210, 49875, 46842, 33201, 83378, 84233, 64708, 85041, or 84234 gene, can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

47476, 67210, 49875, 46842, 33201, 83378, 84233, 64708, 85041, and 84234 Tissue Typing

47476, 67210, 49875, 46842, 33201, 83378, 84233, 64708, 85041, or 84234 sequences can be used to identify individuals from biological samples using, e.g., restriction fragment length polymorphism (RFLP). In this technique, an individual's genomic DNA is digested with one or more restriction enzymes, the fragments separated, e.g., in a Southern blot, and probed to yield bands for identification. The sequences of the present invention are useful as additional DNA markers for RFLP (described in U.S. Pat. No. 5,272,057).

Furthermore, the sequences of the present invention can also be used to determine the actual base-by-base DNA sequence of selected portions of an individual's genome. Thus, the 47476, 67210, 49875, 46842, 33201, 83378, 84233, 64708, 85041, or 84234 nucleotide sequences described herein can be used to prepare two PCR primers from the 5′ and 3′ ends of the sequences. These primers can then be used to amplify an individual's DNA and subsequently sequence it. Panels of corresponding DNA sequences from individuals, prepared in this manner, can provide unique individual identifications, as each individual will have a unique set of such DNA sequences due to allelic differences.

Allelic variation occurs to some degree in the coding regions of these sequences, and to a greater degree in the noncoding regions. Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences of SEQ ID NO:1, SEQ ID NO:4, SEQ ID NO:7, SEQ ID NO:10, SEQ ID NO:13, SEQ ID NO:16, SEQ ID NO:19, SEQ ID NO:22, SEQ ID NO:25, or SEQ ID NO:28 can provide positive individual identification with a panel of perhaps 10 to 1,000 primers which each yield a noncoding amplified sequence of 100 bases. If predicted coding sequences, such as those in SEQ ID NO:3, SEQ ID NO:6, SEQ ID NO:9, SEQ ID NO:12, SEQ ID NO:15, SEQ ID NO:18, SEQ ID NO:21, SEQ ID NO:24, SEQ ID NO:27, or SEQ ID NO:30 are used, a more appropriate number of primers for positive individual identification would be 500-2,000.

If a panel of reagents from 47476, 67210, 49875, 46842, 33201, 83378, 84233, 64708, 85041, or 84234 nucleotide sequences described herein is used to generate a unique identification database for an individual, those same reagents can later be used to identify tissue from that individual. Using the unique identification database, positive identification of the individual, living or dead, can be made from extremely small tissue samples.

Use of Partial 47476, 67210, 49875, 46842, 33201, 83378, 84233, 64708, 85041, or 84234 Sequences in Forensic Biology

DNA-based identification techniques can also be used in forensic biology. To make such an identification, PCR technology can be used to amplify DNA sequences taken from very small biological samples such as tissues, e.g., hair or skin, or body fluids, e.g., blood, saliva, or semen found at a crime scene. The amplified sequence can then be compared to a standard, thereby allowing identification of the origin of the biological sample.

The sequences of the present invention can be used to provide polynucleotide reagents, e.g., PCR primers, targeted to specific loci in the human genome, which can enhance the reliability of DNA-based forensic identifications by, for example, providing another “identification marker” (i.e. another DNA sequence that is unique to a particular individual). As mentioned above, actual base sequence information can be used for identification as an accurate alternative to patterns formed by restriction enzyme generated fragments. Sequences targeted to noncoding regions of SEQ ID NO:1, SEQ ID NO:4, SEQ ID NO:7, SEQ ID NO:10, SEQ ID NO:13, SEQ ID NO:16, SEQ ID NO:19, SEQ ID NO:22, SEQ ID NO:25, or SEQ ID NO:28 (e.g., fragments derived from the noncoding regions of SEQ ID NO:1, SEQ ID NO:4, SEQ ID NO:7, SEQ ID NO:10, SEQ ID NO:13, SEQ ID NO:16, SEQ ID NO:19, SEQ ID NO:22, SEQ ID NO:25, or SEQ ID NO:28 having a length of at least 20 bases, preferably at least 30 bases) are particularly appropriate for this use.

The 47476, 67210, 49875, 46842, 33201, 83378, 84233, 64708, 85041, or 84234 nucleotide sequences described herein can further be used to provide polynucleotide reagents, e.g., labeled or labelable probes which can be used in, for example, an in situ hybridization technique, to identify a specific tissue. This can be very useful in cases where a forensic pathologist is presented with a tissue of unknown origin. Panels of such 47476, 67210, 49875, 46842, 33201, 83378, 84233, 64708, 85041, or 84234 probes can be used to identify tissue by species and/or by organ type.

In a similar fashion, these reagents, e.g., 47476, 67210, 49875, 46842, 33201, 83378, 84233, 64708, 85041, or 84234 primers or probes can be used to screen tissue culture for contamination (i.e. screen for the presence of a mixture of different types of cells in a culture).

Predictive Medicine of 47476, 67210, 49875, 46842, 33201, 83378, 84233, 64708, 85041, and 84234

The present invention also pertains to the field of predictive medicine in which diagnostic assays, prognostic assays, and monitoring clinical trials are used for prognostic (predictive) purposes to thereby treat an individual.

Generally, the invention provides, a method of determining if a subject is at risk for a disorder related to a lesion in or the misexpression of a gene which encodes 47476, 67210, 49875, 46842, 33201, 83378, 84233, 64708, 85041, or 84234.

Such disorders include, e.g., a disorder associated with the misexpression of 47476, 67210, 49875, 46842, 33201, 83378, 84233, 64708, 85041, or 84234 gene, e.g., an immunological disorder, neurological disorder, metabolic disorder, cellular proliferation and/or differentiation disorder, disorder of metal ion imbalance, a protein trafficing disorder, or a cardiovascular disorder.

The method includes one or more of the following:

detecting, in a tissue of the subject, the presence or absence of a mutation which affects the expression of the 47476, 67210, 49875, 46842, 33201, 83378, 84233, 64708, 85041, or 84234 gene, or detecting the presence or absence of a mutation in a region which controls the expression of the gene, e.g., a mutation in the 5′ control region;

detecting, in a tissue of the subject, the presence or absence of a mutation which alters the structure of the 47476, 67210, 49875, 46842, 33201, 83378, 84233, 64708, 85041, or 84234 gene;

detecting, in a tissue of the subject, the misexpression of the 47476, 67210, 49875, 46842, 33201, 83378, 84233, 64708, 85041, or 84234 gene, at the mRNA level, e.g., detecting a non-wild type level of a mRNA;

detecting, in a tissue of the subject, the misexpression of the gene, at the protein level, e.g., detecting a non-wild type level of a 47476, 67210, 49875, 46842, 33201, 83378, 84233, 64708, 85041, or 84234 polypeptide.

In preferred embodiments the method includes: ascertaining the existence of at least one of: a deletion of one or more nucleotides from the 47476, 67210, 49875, 46842, 33201, 83378, 84233, 64708, 85041, or 84234 gene; an insertion of one or more nucleotides into the gene, a point mutation, e.g., a substitution of one or more nucleotides of the gene, a gross chromosomal rearrangement of the gene, e.g., a translocation, inversion, or deletion.

For example, detecting the genetic lesion can include: (i) providing a probe/primer including an oligonucleotide containing a region of nucleotide sequence which hybridizes to a sense or antisense sequence from SEQ ID NO:1, SEQ ID NO:4, SEQ ID NO:7, SEQ ID NO:10, SEQ ID NO:13, SEQ ID NO:16, SEQ ID NO:19, SEQ ID NO:22, SEQ ID NO:25, or SEQ ID NO:28, or naturally occurring mutants thereof or 5′ or 3′ flanking sequences naturally associated with the 47476, 67210, 49875, 46842, 33201, 83378, 84233, 64708, 85041, or 84234 gene; (ii) exposing the probe/primer to nucleic acid of the tissue; and detecting, by hybridization, e.g., in situ hybridization, of the probe/primer to the nucleic acid, the presence or absence of the genetic lesion.

In preferred embodiments detecting the misexpression includes ascertaining the existence of at least one of: an alteration in the level of a messenger RNA transcript of the 47476, 67210, 49875, 46842, 33201, 83378, 84233, 64708, 85041, or 84234 gene; the presence of a non-wild type splicing pattern of a messenger RNA transcript of the gene; or a non-wild type level of 47476, 67210, 49875, 46842, 33201, 83378, 84233, 64708, 85041, or 84234.

Methods of the invention can be used prenatally or to determine if a subject's offspring will be at risk for a disorder.

In preferred embodiments the method includes determining the structure of a 47476, 67210, 49875, 46842, 33201, 83378, 84233, 64708, 85041, or 84234 gene, an abnormal structure being indicative of risk for the disorder.

In preferred embodiments the method includes contacting a sample from the subject with an antibody to the 47476, 67210, 49875, 46842, 33201, 83378, 84233, 64708, 85041, or 84234 protein or a nucleic acid, which hybridizes specifically with the gene. These and other embodiments are discussed below.

Diagnostic and Prognostic Assays of 47476, 67210, 49875, 46842, 33201, 83378, 84233, 64708, 85041, and 84234

Diagnostic and prognostic assays of the invention include method for assessing the expression level of 47476, 67210, 49875, 46842, 33201, 83378, 84233, 64708, 85041, or 84234 molecules and for identifying variations and mutations in the sequence of 47476, 67210, 49875, 46842, 33201, 83378, 84233, 64708, 85041, or 84234 molecules.

Expression Monitoring and Profiling:

The presence, level, or absence of 47476, 67210, 49875, 46842, 33201, 83378, 84233, 64708, 85041, or 84234 protein or nucleic acid in a biological sample can be evaluated by obtaining a biological sample from a test subject and contacting the biological sample with a compound or an agent capable of detecting 47476, 67210, 49875, 46842, 33201, 83378, 84233, 64708, 85041, or 84234 protein or nucleic acid (e.g., mRNA, genomic DNA) that encodes 47476, 67210, 49875, 46842, 33201, 83378, 84233, 64708, 85041, or 84234 protein such that the presence of 47476, 67210, 49875, 46842, 33201, 83378, 84233, 64708, 85041, or 84234 protein or nucleic acid is detected in the biological sample. The term “biological sample” includes tissues, cells and biological fluids isolated from a subject, as well as tissues, cells and fluids present within a subject. A preferred biological sample is serum. The level of expression of the 47476, 67210, 49875, 46842, 33201, 83378, 84233, 64708, 85041, or 84234 gene can be measured in a number of ways, including, but not limited to: measuring the mRNA encoded by the 47476, 67210, 49875, 46842, 33201, 83378, 84233, 64708, 85041, or 84234 genes; measuring the amount of protein encoded by the 47476, 67210, 49875, 46842, 33201, 83378, 84233, 64708, 85041, or 84234 genes; or measuring the activity of the protein encoded by the 47476, 67210, 49875, 46842, 33201, 83378, 84233, 64708, 85041, or 84234 genes.

The level of mRNA corresponding to the 47476, 67210, 49875, 46842, 33201, 83378, 84233, 64708, 85041, or 84234 gene in a cell can be determined both by in situ and by in vitro formats.

The isolated mRNA can be used in hybridization or amplification assays that include, but are not limited to, Southern or Northern analyses, polymerase chain reaction analyses and probe arrays. One preferred diagnostic method for the detection of mRNA levels involves contacting the isolated mRNA with a nucleic acid molecule (probe) that can hybridize to the mRNA encoded by the gene being detected. The nucleic acid probe can be, for example, a full-length 47476, 67210, 49875, 46842, 33201, 83378, 84233, 64708, 85041, or 84234 nucleic acid, such as the nucleic acid of SEQ ID NO:1, SEQ ID NO:4, SEQ ID NO:7, SEQ ID NO:10, SEQ ID NO:13, SEQ ID NO:16, SEQ ID NO:19, SEQ ID NO:22, SEQ ID NO:25, or SEQ ID NO:28, or a portion thereof, such as an oligonucleotide of at least 7, 15, 30, 50, 100, 250 or 500 nucleotides in length and sufficient to specifically hybridize under stringent conditions to 47476, 67210, 49875, 46842, 33201, 83378, 84233, 64708, 85041, or 84234 mRNA or genomic DNA. The probe can be disposed on an address of an array, e.g., an array described below. Other suitable probes for use in the diagnostic assays are described herein.

In one format, mRNA (or cDNA) is immobilized on a surface and contacted with the probes, for example by running the isolated mRNA on an agarose gel and transferring the mRNA from the gel to a membrane, such as nitrocellulose. In an alternative format, the probes are immobilized on a surface and the mRNA (or cDNA) is contacted with the probes, for example, in a two-dimensional gene chip array described below. A skilled artisan can adapt known mRNA detection methods for use in detecting the level of mRNA encoded by the 47476, 67210, 49875, 46842, 33201, 83378, 84233, 64708, 85041, or 84234 genes.

The level of mRNA in a sample that is encoded by one of 47476, 67210, 49875, 46842, 33201, 83378, 84233, 64708, 85041, or 84234 can be evaluated with nucleic acid amplification, e.g., by rtPCR (Mullis (1987) U.S. Pat. No. 4,683,202), ligase chain reaction (Barany (1991) Proc. Natl. Acad. Sci. USA 88:189-193), self sustained sequence replication (Guatelli et al., (1990) Proc. Natl. Acad. Sci. USA 87:1874-1878), transcriptional amplification system (Kwoh et al., (1989), Proc. Natl. Acad. Sci. USA 86:1173-1177), Q-Beta Replicase (Lizardi et al., (1988) Bio/Technology 6:1197), rolling circle replication (Lizardi et al., U.S. Pat. No. 5,854,033) or any other nucleic acid amplification method, followed by the detection of the amplified molecules using techniques known in the art. As used herein, amplification primers are defined as being a pair of nucleic acid molecules that can anneal to 5′ or 3′ regions of a gene (plus and minus strands, respectively, or vice-versa) and contain a short region in between. In general, amplification primers are from about 10 to 30 nucleotides in length and flank a region from about 50 to 200 nucleotides in length. Under appropriate conditions and with appropriate reagents, such primers permit the amplification of a nucleic acid molecule comprising the nucleotide sequence flanked by the primers.

For in situ methods, a cell or tissue sample can be prepared/processed and immobilized on a support, typically a glass slide, and then contacted with a probe that can hybridize to mRNA that encodes the 47476, 67210, 49875, 46842, 33201, 83378, 84233, 64708, 85041, or 84234 gene being analyzed.

In another embodiment, the methods further contacting a control sample with a compound or agent capable of detecting 47476, 67210, 49875, 46842, 33201, 83378, 84233, 64708, 85041, or 84234 mRNA, or genomic DNA, and comparing the presence of 47476, 67210, 49875, 46842, 33201, 83378, 84233, 64708, 85041, or 84234 mRNA or genomic DNA in the control sample with the presence of 47476, 67210, 49875, 46842, 33201, 83378, 84233, 64708, 85041, or 84234 mRNA or genomic DNA in the test sample. In still another embodiment, serial analysis of gene expression, as described in U.S. Pat. No. 5,695,937, is used to detect 47476, 67210, 49875, 46842, 33201, 83378, 84233, 64708, 85041, or 84234 transcript levels.

A variety of methods can be used to determine the level of protein encoded by 47476, 67210, 49875, 46842, 33201, 83378, 84233, 64708, 85041, or 84234. In general, these methods include contacting an agent that selectively binds to the protein, such as an antibody with a sample, to evaluate the level of protein in the sample. In a preferred embodiment, the antibody bears a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with a detectable substance. Examples of detectable substances are provided herein.

The detection methods can be used to detect 47476, 67210, 49875, 46842, 33201, 83378, 84233, 64708, 85041, or 84234 protein in a biological sample in vitro as well as in vivo. In vitro techniques for detection of 47476, 67210, 49875, 46842, 33201, 83378, 84233, 64708, 85041, or 84234 protein include enzyme linked immunosorbent assays (ELISAs), immunoprecipitations, immunofluorescence, enzyme immunoassay (EIA), radioimmunoassay (RIA), and Western blot analysis. In vivo techniques for detection of 47476, 67210, 49875, 46842, 33201, 83378, 84233, 64708, 85041, or 84234 protein include introducing into a subject a labeled anti-47476, 67210, 49875, 46842, 33201, 83378, 84233, 64708, 85041, or 84234 antibody. For example, the antibody can be labeled with a radioactive marker whose presence and location in a subject can be detected by standard imaging techniques. In another embodiment, the sample is labeled, e.g., biotinylated and then contacted to the antibody, e.g., an anti-47476, 67210, 49875, 46842, 33201, 83378, 84233, 64708, 85041, or 84234 antibody positioned on an antibody array (as described below). The sample can be detected, e.g., with avidin coupled to a fluorescent label.

In another embodiment, the methods further include contacting the control sample with a compound or agent capable of detecting 47476, 67210, 49875, 46842, 33201, 83378, 84233, 64708, 85041, or 84234 protein, and comparing the presence of 47476, 67210, 49875, 46842, 33201, 83378, 84233, 64708, 85041, or 84234 protein in the control sample with the presence of 47476, 67210, 49875, 46842, 33201, 83378, 84233, 64708, 85041, or 84234 protein in the test sample.

The invention also includes kits for detecting the presence of 47476, 67210, 49875, 46842, 33201, 83378, 84233, 64708, 85041, or 84234 in a biological sample. For example, the kit can include a compound or agent capable of detecting 47476, 67210, 49875, 46842, 33201, 83378, 84233, 64708, 85041, or 84234 protein or mRNA in a biological sample; and a standard. The compound or agent can be packaged in a suitable container. The kit can further comprise instructions for using the kit to detect 47476, 67210, 49875, 46842, 33201, 83378, 84233, 64708, 85041, or 84234 protein or nucleic acid.

For antibody-based kits, the kit can include: (1) a first antibody (e.g., attached to a solid support) which binds to a polypeptide corresponding to a marker of the invention; and, optionally, (2) a second, different antibody which binds to either the polypeptide or the first antibody and is conjugated to a detectable agent.

For oligonucleotide-based kits, the kit can include: (1) an oligonucleotide, e.g., a detectably labeled oligonucleotide, which hybridizes to a nucleic acid sequence encoding a polypeptide corresponding to a marker of the invention or (2) a pair of primers useful for amplifying a nucleic acid molecule corresponding to a marker of the invention. The kit can also includes a buffering agent, a preservative, or a protein stabilizing agent. The kit can also includes components necessary for detecting the detectable agent (e.g., an enzyme or a substrate). The kit can also contain a control sample or a series of control samples which can be assayed and compared to the test sample contained. Each component of the kit can be enclosed within an individual container and all of the various containers can be within a single package, along with instructions for interpreting the results of the assays performed using the kit.

The diagnostic methods described herein can identify subjects having, or at risk of developing, a disease or disorder associated with misexpressed or aberrant or unwanted 47476, 67210, 49875, 46842, 33201, 83378, 84233, 64708, 85041, or 84234 expression or activity. As used herein, the term “unwanted” includes an unwanted phenomenon involved in a biological response such as an immunological disorder, neurological disorder, metabolic disorder, cellular proliferation and/or differentiation disorder, disorder of metal ion imbalance, a protein trafficing disorder, or a cardiovascular disorder.

In one embodiment, a disease or disorder associated with aberrant or unwanted 47476, 67210, 49875, 46842, 33201, 83378, 84233, 64708, 85041, or 84234 expression or activity is identified. A test sample is obtained from a subject and 47476, 67210, 49875, 46842, 33201, 83378, 84233, 64708, 85041, or 84234 protein or nucleic acid (e.g., mRNA or genomic DNA) is evaluated, wherein the level, e.g., the presence or absence, of 47476, 67210, 49875, 46842, 33201, 83378, 84233, 64708, 85041, or 84234 protein or nucleic acid is diagnostic for a subject having or at risk of developing a disease or disorder associated with aberrant or unwanted 47476, 67210, 49875, 46842, 33201, 83378, 84233, 64708, 85041, or 84234 expression or activity. As used herein, a “test sample” refers to a biological sample obtained from a subject of interest, including a biological fluid (e.g., serum), cell sample, or tissue.

The prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant or unwanted 47476, 67210, 49875, 46842, 33201, 83378, 84233, 64708, 85041, or 84234 expression or activity. For example, such methods can be used to determine whether a subject can be effectively treated with an agent for an immunological disorder, neurological disorder, metabolic disorder, cellular proliferation and/or differentiation disorder, disorder of metal ion imbalance, a protein trafficing disorder disorder, or a cardiovascular disorder.

In another aspect, the invention features a computer medium having a plurality of digitally encoded data records. Each data record includes a value representing the level of expression of 47476, 67210, 49875, 46842, 33201, 83378, 84233, 64708, 85041, or 84234 in a sample, and a descriptor of the sample. The descriptor of the sample can be an identifier of the sample, a subject from which the sample was derived (e.g., a patient), a diagnosis, or a treatment (e.g., a preferred treatment). In a preferred embodiment, the data record further includes values representing the level of expression of genes other than 47476, 67210, 49875, 46842, 33201, 83378, 84233, 64708, 85041, or 84234 (e.g., other genes associated with a 47476, 67210, 49875, 46842, 33201, 83378, 84233, 64708, 85041, or 84234-disorder, or other genes on an array). The data record can be structured as a table, e.g., a table that is part of a database such as a relational database (e.g., a SQL database of the Oracle or Sybase database environments).

Also featured is a method of evaluating a sample. The method includes providing a sample, e.g., from the subject, and determining a gene expression profile of the sample, wherein the profile includes a value representing the level of 47476, 67210, 49875, 46842, 33201, 83378, 84233, 64708, 85041, or 84234 expression. The method can further include comparing the value or the profile (i.e., multiple values) to a reference value or reference profile. The gene expression profile of the sample can be obtained by any of the methods described herein (e.g., by providing a nucleic acid from the sample and contacting the nucleic acid to an array). The method can be used to diagnose an immunological disorder, neurological disorder, metabolic disorder, cellular proliferation and/or differentiation disorder, disorder of metal ion imbalance, a protein trafficing disorder disorder, or a cardiovascular disorder in a subject wherein an increase or decrease in 47476, 67210, 49875, 46842, 33201, 83378, 84233, 64708, 85041, or 84234 expression is an indication that the subject has or is disposed to having an immunological disorder, neurological disorder, metabolic disorder, cellular proliferation and/or differentiation disorder, disorder of metal ion imbalance, a protein trafficing disorder, or a cardiovascular disorder. The method can be used to monitor a treatment for an immunological disorder, neurological disorder, metabolic disorder, cellular proliferation and/or differentiation disorder, disorder of metal ion imbalance, a protein trafficing disorder, or a cardiovascular disorder in a subject. For example, the gene expression profile can be determined for a sample from a subject undergoing treatment. The profile can be compared to a reference profile or to a profile obtained from the subject prior to treatment or prior to onset of the disorder (see, e.g., Golub et al. (1999) Science 286:531).

In yet another aspect, the invention features a method of evaluating a test compound (see also, “Screening Assays”, above). The method includes providing a cell and a test compound; contacting the test compound to the cell; obtaining a subject expression profile for the contacted cell; and comparing the subject expression profile to one or more reference profiles. The profiles include a value representing the level of 47476, 67210, 49875, 46842, 33201, 83378, 84233, 64708, 85041, or 84234 expression. In a preferred embodiment, the subject expression profile is compared to a target profile, e.g., a profile for a normal cell or for desired condition of a cell. The test compound is evaluated favorably if the subject expression profile is more similar to the target profile than an expression profile obtained from an uncontacted cell.

In another aspect, the invention features, a method of evaluating a subject. The method includes: a) obtaining a sample from a subject, e.g., from a caregiver, e.g., a caregiver who obtains the sample from the subject; b) determining a subject expression profile for the sample. Optionally, the method further includes either or both of steps: c) comparing the subject expression profile to one or more reference expression profiles; and d) selecting the reference profile most similar to the subject reference profile. The subject expression profile and the reference profiles include a value representing the level of 47476, 67210, 49875, 46842, 33201, 83378, 84233, 64708, 85041, or 84234 expression. A variety of routine statistical measures can be used to compare two reference profiles. One possible metric is the length of the distance vector that is the difference between the two profiles. Each of the subject and reference profile is represented as a multi-dimensional vector, wherein each dimension is a value in the profile.

The method can further include transmitting a result to a caregiver. The result can be the subject expression profile, a result of a comparison of the subject expression profile with another profile, a most similar reference profile, or a descriptor of any of the aforementioned. The result can be transmitted across a computer network, e.g., the result can be in the form of a computer transmission, e.g., a computer data signal embedded in a carrier wave.

Also featured is a computer medium having executable code for effecting the following steps: receive a subject expression profile; access a database of reference expression profiles; and either i) select a matching reference profile most similar to the subject expression profile or ii) determine at least one comparison score for the similarity of the subject expression profile to at least one reference profile. The subject expression profile, and the reference expression profiles each include a value representing the level of 47476, 67210, 49875, 46842, 33201, 83378, 84233, 64708, 85041, or 84234 expression.

47476, 67210, 49875, 46842, 33201, 83378, 84233, 64708, 85041, and 84234 Arrays and uses Thereof

In another aspect, the invention features an array that includes a substrate having a plurality of addresses. At least one address of the plurality includes a capture probe that binds specifically to a 47476, 67210, 49875, 46842, 33201, 83378, 84233, 64708, 85041, or 84234 molecule (e.g., a 47476, 67210, 49875, 46842, 33201, 83378, 84233, 64708, 85041, or 84234 nucleic acid or a 47476, 67210, 49875, 46842, 33201, 83378, 84233, 64708, 85041, or 84234 polypeptide). The array can have a density of at least than 10, 50, 100, 200, 500, 1,000, 2,000, or 10,000 or more addresses/cm², and ranges between. In a preferred embodiment, the plurality of addresses includes at least 10, 100, 500, 1,000, 5,000, 10,000, 50,000 addresses. In a preferred embodiment, the plurality of addresses includes equal to or less than 10, 100, 500, 1,000, 5,000, 10,000, or 50,000 addresses. The substrate can be a two-dimensional substrate such as a glass slide, a wafer (e.g., silica or plastic), a mass spectroscopy plate, or a three-dimensional substrate such as a gel pad. Addresses in addition to address of the plurality can be disposed on the array.

In a preferred embodiment, at least one address of the plurality includes a nucleic acid capture probe that hybridizes specifically to a 47476, 67210, 49875, 46842, 33201, 83378, 84233, 64708, 85041, or 84234 nucleic acid, e.g., the sense or anti-sense strand. In one preferred embodiment, a subset of addresses of the plurality of addresses has a nucleic acid capture probe for 47476, 67210, 49875, 46842, 33201, 83378, 84233, 64708, 85041, or 84234. Each address of the subset can include a capture probe that hybridizes to a different region of a 47476, 67210, 49875, 46842, 33201, 83378, 84233, 64708, 85041, or 84234 nucleic acid. In another preferred embodiment, addresses of the subset include a capture probe for a 47476, 67210, 49875, 46842, 33201, 83378, 84233, 64708, 85041, or 84234 nucleic acid. Each address of the subset is unique, overlapping, and complementary to a different variant of 47476, 67210, 49875, 46842, 33201, 83378, 84233, 64708, 85041, or 84234 (e.g., an allelic variant, or all possible hypothetical variants). The array can be used to sequence 47476, 67210, 49875, 46842, 33201, 83378, 84233, 64708, 85041, or 84234 by hybridization (see, e.g., U.S. Pat. No. 5,695,940).

An array can be generated by various methods, e.g., by photolithographic methods (see, e.g., U.S. Pat. Nos. 5,143,854; 5,510,270; and 5,527,681), mechanical methods (e.g., directed-flow methods as described in U.S. Pat. No. 5,384,261), pin-based methods (e.g., as described in U.S. Pat. No. 5,288,514), and bead-based techniques (e.g., as described in PCT US/93/04145).

In another preferred embodiment, at least one address of the plurality includes a polypeptide capture probe that binds specifically to a 47476, 67210, 49875, 46842, 33201, 83378, 84233, 64708, 85041, or 84234 polypeptide or fragment thereof. The polypeptide can be a naturally-occurring interaction partner of 47476, 67210, 49875, 46842, 33201, 83378, 84233, 64708, 85041, or 84234 polypeptide. Preferably, the polypeptide is an antibody, e.g., an antibody described herein (see “Anti-47476, 67210, 49875, 46842, 33201, 83378, 84233, 64708, 85041, or 84234 Antibodies,” above), such as a monoclonal antibody or a single-chain antibody.

In another aspect, the invention features a method of analyzing the expression of 47476, 67210, 49875, 46842, 33201, 83378, 84233, 64708, 85041, or 84234. The method includes providing an array as described above; contacting the array with a sample and detecting binding of a 47476, 67210, 49875, 46842, 33201, 83378, 84233, 64708, 85041, or 84234-molecule (e.g., nucleic acid or polypeptide) to the array. In a preferred embodiment, the array is a nucleic acid array. Optionally the method further includes amplifying nucleic acid from the sample prior or during contact with the array.

In another embodiment, the array can be used to assay gene expression in a tissue to ascertain tissue specificity of genes in the array, particularly the expression of 47476, 67210, 49875, 46842, 33201, 83378, 84233, 64708, 85041, or 84234. If a sufficient number of diverse samples is analyzed, clustering (e.g., hierarchical clustering, k-means clustering, Bayesian clustering and the like) can be used to identify other genes which are co-regulated with 47476, 67210, 49875, 46842, 33201, 83378, 84233, 64708, 85041, or 84234. For example, the array can be used for the quantitation of the expression of multiple genes. Thus, not only tissue specificity, but also the level of expression of a battery of genes in the tissue is ascertained. Quantitative data can be used to group (e.g., cluster) genes on the basis of their tissue expression per se and level of expression in that tissue.

For example, array analysis of gene expression can be used to assess the effect of cell-cell interactions on 47476, 67210, 49875, 46842, 33201, 83378, 84233, 64708, 85041, or 84234 expression. A first tissue can be perturbed and nucleic acid from a second tissue that interacts with the first tissue can be analyzed. In this context, the effect of one cell type on another cell type in response to a biological stimulus can be determined, e.g., to monitor the effect of cell-cell interaction at the level of gene expression.

In another embodiment, cells are contacted with a therapeutic agent. The expression profile of the cells is determined using the array, and the expression profile is compared to the profile of like cells not contacted with the agent. For example, the assay can be used to determine or analyze the molecular basis of an undesirable effect of the therapeutic agent. If an agent is administered therapeutically to treat one cell type but has an undesirable effect on another cell type, the invention provides an assay to determine the molecular basis of the undesirable effect and thus provides the opportunity to co-administer a counteracting agent or otherwise treat the undesired effect. Similarly, even within a single cell type, undesirable biological effects can be determined at the molecular level. Thus, the effects of an agent on expression of other than the target gene can be ascertained and counteracted.

In another embodiment, the array can be used to monitor expression of one or more genes in the array with respect to time. For example, samples obtained from different time points can be probed with the array. Such analysis can identify and/or characterize the development of a 47476, 67210, 49875, 46842, 33201, 83378, 84233, 64708, 85041, or 84234-associated disease or disorder; and processes, such as a cellular transformation associated with a 0.47476, 67210, 49875, 46842, 33201, 83378, 84233, 64708, 85041, or 84234-associated disease or disorder. The method can also evaluate the treatment and/or progression of a 47476, 67210, 49875, 46842, 33201, 83378, 84233, 64708, 85041, or 84234-associated disease or disorder The array is also useful for ascertaining differential expression patterns of one or more genes in normal and abnormal cells. This provides a battery of genes (e.g., including 47476, 67210, 49875, 46842, 33201, 83378, 84233, 64708, 85041, or 84234) that could serve as a molecular target for diagnosis or therapeutic intervention.

In another aspect, the invention features an array having a plurality of addresses. Each address of the plurality includes a unique polypeptide. At least one address of the plurality has disposed thereon a 47476, 67210, 49875, 46842, 33201, 83378, 84233, 64708, 85041, or 84234 polypeptide or fragment thereof. Methods of producing polypeptide arrays are described in the art, e.g., in De Wildt et al. (2000). Nature Biotech. 18, 989-994; Lueking et al. (1999). Anal. Biochem. 270, 103-111; Ge, H. (2000). Nucleic Acids Res. 28, e3, I-VII; MacBeath, G., and Schreiber, S. L. (2000). Science 289, 1760-1763; and WO 99/51773A1. In a preferred embodiment, each addresses of the plurality has disposed thereon a polypeptide at least 60, 70, 80, 85, 90, 95 or 99% identical to a 47476, 67210, 49875, 46842, 33201, 83378, 84233, 64708, 85041, or 84234 polypeptide or fragment thereof. For example, multiple variants of a 47476, 67210, 49875, 46842, 33201, 83378, 84233, 64708, 85041, or 84234 polypeptide (e.g., encoded by allelic variants, site-directed mutants, random mutants, or combinatorial mutants) can be disposed at individual addresses of the plurality. Addresses in addition to the address of the plurality can be disposed on the array.

The polypeptide array can be used to detect a 47476, 67210, 49875, 46842, 33201, 83378, 84233, 64708, 85041, or 84234 binding compound, e.g., an antibody in a sample from a subject with specificity for a 47476, 67210, 49875, 46842, 33201, 83378, 84233, 64708, 85041, or 84234 polypeptide or the presence of a 47476, 67210, 49875, 46842, 33201, 83378, 84233, 64708, 85041, or 84234-binding protein or ligand.

The array is also useful for ascertaining the effect of the expression of a gene on the expression of other genes in the same cell or in different cells (e.g., ascertaining the effect of 47476, 67210, 49875, 46842, 33201, 83378, 84233, 64708, 85041, or 84234 expression on the expression of other genes). This provides, for example, for a selection of alternate molecular targets for therapeutic intervention if the ultimate or downstream target cannot be regulated.

In another aspect, the invention features a method of analyzing a plurality of probes. The method is useful, e.g., for analyzing gene expression. The method includes: providing a two dimensional array having a plurality of addresses, each address of the plurality being positionally distinguishable from each other address of the plurality having a unique capture probe, e.g., wherein the capture probes are from a cell or subject which express 47476, 67210, 49875, 46842, 33201, 83378, 84233, 64708, 85041, or 84234 or from a cell or subject in which a 47476, 67210, 49875, 46842, 33201, 83378, 84233, 64708, 85041, or 84234 mediated response has been elicited, e.g., by contact of the cell with 47476, 67210, 49875, 46842, 33201, 83378, 84233, 64708, 85041, or 84234 nucleic acid or protein, or administration to the cell or subject 47476, 67210, 49875, 46842, 33201, 83378, 84233, 64708, 85041, or 84234 nucleic acid or protein; providing a two dimensional array having a plurality of addresses, each address of the plurality being positionally distinguishable from each other address of the plurality, and each address of the plurality having a unique capture probe, e.g., wherein the capture probes are from a cell or subject which does not express 47476, 67210, 49875, 46842, 33201, 83378, 84233, 64708, 85041, or 84234 (or does not express as highly as in the case of the 47476, 67210, 49875, 46842, 33201, 83378, 84233, 64708, 85041, or 84234 positive plurality of capture probes) or from a cell or subject which in which a 47476, 67210, 49875, 46842, 33201, 83378, 84233, 64708, 85041, or 84234 mediated response has not been elicited (or has been elicited to a lesser extent than in the first sample); contacting the array with one or more inquiry probes (which is preferably other than a 47476, 67210, 49875, 46842, 33201, 83378, 84233, 64708, 85041, or 84234 nucleic acid, polypeptide, or antibody), and thereby evaluating the plurality of capture probes. Binding, e.g., in the case of a nucleic acid, hybridization with a capture probe at an address of the plurality, is detected, e.g., by signal generated from a label attached to the nucleic acid, polypeptide, or antibody.

In another aspect, the invention features a method of analyzing a plurality of probes or a sample. The method is useful, e.g., for analyzing gene expression. The method includes: providing a two dimensional array having a plurality of addresses, each address of the plurality being positionally distinguishable from each other address of the plurality having a unique capture probe, contacting the array with a first sample from a cell or subject which express or mis-express 47476, 67210, 49875, 46842, 33201, 83378, 84233, 64708, 85041, or 84234 or from a cell or subject in which a 47476, 67210, 49875, 46842, 33201, 83378, 84233, 64708, 85041, or 84234-mediated response has been elicited, e.g., by contact of the cell with 47476, 67210, 49875, 46842, 33201, 83378, 84233, 64708, 85041, or 84234 nucleic acid or protein, or administration to the cell or subject 47476, 67210, 49875, 46842, 33201, 83378, 84233, 64708, 85041, or 84234 nucleic acid or protein; providing a two dimensional array having a plurality of addresses, each address of the plurality being positionally distinguishable from each other address of the plurality, and each address of the plurality having a unique capture probe, and contacting the array with a second sample from a cell or subject which does not express 47476, 67210, 49875, 46842, 33201, 83378, 84233, 64708, 85041, or 84234 (or does not express as highly as in the case of the 47476, 67210, 49875, 46842, 33201, 83378, 84233, 64708, 85041, or 84234 positive plurality of capture probes) or from a cell or subject which in which a 47476, 67210, 49875, 46842, 33201, 83378, 84233, 64708, 85041, or 84234 mediated response has not been elicited (or has been elicited to a lesser extent than in the first sample); and comparing the binding of the first sample with the binding of the second sample. Binding, e.g., in the case of a nucleic acid, hybridization with a capture probe at an address of the plurality, is detected, e.g., by signal generated from a label attached to the nucleic acid, polypeptide, or antibody. The same array can be used for both samples or different arrays can be used. If different arrays are used the plurality of addresses with capture probes should be present on both arrays.

In another aspect, the invention features a method of analyzing 47476, 67210, 49875, 46842, 33201, 83378, 84233, 64708, 85041, or 84234, e.g., analyzing structure, function, or relatedness to other nucleic acid or amino acid sequences. The method includes: providing a 47476, 67210, 49875, 46842, 33201, 83378, 84233, 64708, 85041, or 84234 nucleic acid or amino acid sequence; comparing the 47476, 67210, 49875, 46842, 33201, 83378, 84233, 64708, 85041, or 84234 sequence with one or more preferably a plurality of sequences from a collection of sequences, e.g., a nucleic acid or protein sequence database; to thereby analyze 47476, 67210, 49875, 46842, 33201, 83378, 84233, 4708, 85041, or 84234.

Detection of 47476, 67210, 49875, 46842, 33201, 83378, 84233, 64708, 85041, and 84234 Variations or Mutations

The methods of the invention can also be used to detect genetic alterations in a 47476, 67210, 49875, 46842, 33201, 83378, 84233, 64708, 85041, or 84234 gene, thereby determining if a subject with the altered gene is at risk for a disorder characterized by misregulation in 47476, 67210, 49875, 46842, 33201, 83378, 84233, 64708, 85041, or 84234 protein activity or nucleic acid expression, such as an immunological disorder, neurological disorder, metabolic disorder, cellular proliferation and/or differentiation disorder, disorder of metal ion imbalance, a protein trafficking disorder, or a cardiovascular disorder. In preferred embodiments, the methods include detecting, in a sample from the subject, the presence or absence of a genetic alteration characterized by at least one of an alteration affecting the integrity of a gene encoding a 47476, 67210, 49875, 46842, 33201, 83378, 84233, 64708, 85041, or 84234-protein, or the mis-expression of the 47476, 67210, 49875, 46842, 33201, 83378, 84233, 64708, 85041, or 84234 gene. For example, such genetic alterations can be detected by ascertaining the existence of at least one of 1) a deletion of one or more nucleotides from a 47476, 67210, 49875, 46842, 33201, 83378, 84233, 64708, 85041, or 84234 gene; 2) an addition of one or more nucleotides to a 47476, 67210, 49875, 46842, 33201, 83378, 84233, 64708, 85041, or 84234 gene; 3) a substitution of one or more nucleotides of a 47476, 67210, 49875, 46842, 33201, 83378, 84233, 64708, 85041, or 84234 gene, 4) a chromosomal rearrangement of a 47476, 67210, 49875, 46842, 33201, 83378, 84233, 64708, 85041, or 84234 gene; 5) an alteration in the level of a messenger RNA transcript of a 47476, 67210, 49875, 46842, 33201, 83378, 84233, 64708, 85041, or 84234 gene, 6) aberrant modification of a 47476, 67210, 49875, 46842, 33201, 83378, 84233, 64708, 85041, or 84234 gene, such as of the methylation pattern of the genomic DNA, 7) the presence of a non-wild type splicing pattern of a messenger RNA transcript of a 47476, 67210, 49875, 46842, 33201, 83378, 84233, 64708, 85041, or 84234 gene, 8) a non-wild type level of a 47476, 67210, 49875, 46842, 33201, 83378, 84233, 64708, 85041, or 84234-protein, 9) allelic loss of a 47476, 67210, 49875, 46842, 33201, 83378, 84233, 64708, 85041, or 84234 gene, and 10) inappropriate post-translational modification of a 47476, 67210, 49875, 46842, 33201, 83378, 84233, 64708, 85041, or 84234-protein.

An alteration can be detected without a probe/primer in a polymerase chain reaction, such as anchor PCR or RACE PCR, or, alternatively, in a ligation chain reaction (LCR), the latter of which can be particularly useful for detecting point mutations in the 47476, 67210, 49875, 46842, 33201, 83378, 84233, 64708, 85041, or 84234-gene. This method can include the steps of collecting a sample of cells from a subject, isolating nucleic acid (e.g., genomic, mRNA or both) from the sample, contacting the nucleic acid sample with one or more primers which specifically hybridize to a 47476, 67210, 49875, 46842, 33201, 83378, 84233, 64708, 85041, or 84234 gene under conditions such that hybridization and amplification of the 47476, 67210, 49875, 46842, 33201, 83378, 84233, 64708, 85041, or 84234-gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein. Alternatively, other amplification methods described herein or known in the art can be used.

In another embodiment, mutations in a 47476, 67210, 49875, 46842, 33201, 83378, 84233, 64708, 85041, or 84234 gene from a sample cell can be identified by detecting alterations in restriction enzyme cleavage patterns. For example, sample and control DNA is isolated, amplified (optionally), digested with one or more restriction endonucleases, and fragment length sizes are determined, e.g., by gel electrophoresis and compared. Differences in fragment length sizes between sample and control DNA indicates mutations in the sample DNA. Moreover, the use of sequence specific ribozymes (see, for example, U.S. Pat. No. 5,498,531) can be used to score for the presence of specific mutations by development or loss of a ribozyme cleavage site.

In other embodiments, genetic mutations in 47476, 67210, 49875, 46842, 33201, 83378, 84233, 64708, 85041, or 84234 can be identified by hybridizing a sample and control nucleic acids, e.g., DNA or RNA, two-dimensional arrays, e.g., chip based arrays. Such arrays include a plurality of addresses, each of which is positionally distinguishable from the other. A different probe is located at each address of the plurality. A probe can be complementary to a region of a 47476, 67210, 49875, 46842, 33201, 83378, 84233, 64708, 85041, or 84234 nucleic acid or a putative variant (e.g., allelic variant) thereof. A probe can have one or more mismatches to a region of a 47476, 67210, 49875, 46842, 33201, 83378, 84233, 64708, 85041, or 84234 nucleic acid (e.g., a destabilizing mismatch). The arrays can have a high density of addresses, e.g., can contain hundreds or thousands of oligonucleotides probes (Cronin, M. T. et al. (1996) Human Mutation 7: 244-255; Kozal, M. J. et al. (1996) Nature Medicine 2: 753-759). For example, genetic mutations in 47476, 67210, 49875, 46842, 33201, 83378, 84233, 64708, 85041, or 84234 can be identified in two-dimensional arrays containing light-generated DNA probes as described in Cronin, M. T. et al. supra. Briefly, a first hybridization array of probes can be used to scan through long stretches of DNA in a sample and control to identify base changes between the sequences by making linear arrays of sequential overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the 47476, 67210, 49875, 46842, 33201, 83378, 84233, 64708, 85041, or 84234 gene and detect mutations by comparing the sequence of the sample 47476, 67210, 49875, 46842, 33201, 83378, 84233, 64708, 85041, or 84234 with the corresponding wild-type (control) sequence. Automated sequencing procedures can be utilized when performing the diagnostic assays ((1995) Biotechniques 19:448), including sequencing by mass spectrometry.

Other methods for detecting mutations in the 47476, 67210, 49875, 46842, 33201, 83378, 84233, 64708, 85041, or 84234 gene include methods in which protection from cleavage agents is used to detect mismatched bases in RNA/RNA or RNA/DNA heteroduplexes (Myers et al. (1985) Science 230:1242; Cotton et al. (1988) Proc. Natl. Acad Sci USA 85:4397; Saleeba et al. (1992) Methods Enzymol. 217:286-295).

In still another embodiment, the mismatch cleavage reaction employs one or more proteins that recognize mismatched base pairs in double-stranded DNA (so called “DNA mismatch repair” enzymes) in defined systems for detecting and mapping point mutations in 47476, 67210, 49875, 46842, 33201, 83378, 84233, 64708, 85041, or 84234 cDNAs obtained from samples of cells. For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1662; U.S. Pat. No. 5,459,039).

In other embodiments, alterations in electrophoretic mobility will be used to identify mutations in 47476, 67210, 49875, 46842, 33201, 83378, 84233, 64708, 85041, or 84234 genes. For example, single strand conformation polymorphism (SSCP) may be used to detect differences in electrophoretic mobility between mutant and wild type nucleic acids (Orita et al. (1989) Proc Natl. Acad. Sci USA: 86:2766, see also Cotton (1993) Mutat. Res. 285:125-144; and Hayashi (1992) Genet. Anal. Tech. Appl. 9:73-79). Single-stranded DNA fragments of sample and control 47476, 67210, 49875, 46842, 33201, 83378, 84233, 64708, 85041, or 84234 nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. (1991) Trends Genet 7:5).

In yet another embodiment, the movement of mutant or wild-type fragments in polyacrylamide gels containing a gradient of denaturant is assayed using denaturing gradient gel electrophoresis (DGGE) (Myers et al. (1985) Nature 313:495). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner (1987) Biophys Chem 265:12753).

Examples of other techniques for detecting point mutations include, but are not limited to, selective oligonucleotide hybridization, selective amplification, or selective primer extension (Saiki et al. (1986) Nature 324:163); Saiki et al. (1989) Proc. Natl Acad. Sci USA 86:6230). A further method of detecting point mutations is the chemical ligation of oligonucleotides as described in Xu et al. ((2001) Nature Biotechnol. 19:148). Adjacent oligonucleotides, one of which selectively anneals to the query site, are ligated together if the nucleotide at the query site of the sample nucleic acid is complementary to the query oligonucleotide; ligation can be monitored, e.g., by fluorescent dyes coupled to the oligonucleotides.

Alternatively, allele specific amplification technology that depends on selective PCR amplification may be used in conjunction with the instant invention. Oligonucleotides used as primers for specific amplification may carry the mutation of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent, or reduce polymerase extension (Prossner (1993) Tibtech 11:238). In addition it may be desirable to introduce a novel restriction site in the region of the mutation to create cleavage-based detection (Gasparini et al. (1992) Mol. Cell Probes 6:1). It is anticipated that in certain embodiments amplification may also be performed using Taq ligase for amplification (Barany (1991) Proc. Natl. Acad. Sci USA 88:189). In such cases, ligation will occur only if there is a perfect match at the 3′ end of the 5′ sequence making it possible to detect the presence of a known mutation at a specific site by looking for the presence or absence of amplification.

In another aspect, the invention features a set of oligonucleotides. The set includes a plurality of oligonucleotides, each of which is at least partially complementary (e.g., at least 50%, 60%, 70%, 80%, 90%, 92%, 95%, 97%, 98%, or 99% complementary) to a 47476, 67210, 49875, 46842, 33201, 83378, 84233, 64708, 85041, or 84234 nucleic acid.

In a preferred embodiment the set includes a first and a second oligonucleotide. The first and second oligonucleotide can hybridize to the same or to different locations of SEQ ID NO:1, SEQ ID NO:4, SEQ ID NO:7, SEQ ID NO:10, SEQ ID NO:13, SEQ ID NO:16, SEQ ID NO:19, SEQ ID NO:22, SEQ ID NO:25, or SEQ ID NO:28. Different locations can be different but overlapping, or non-overlapping on the same strand. The first and second oligonucleotide can hybridize to sites on the same or on different strands.

The set can be useful, e.g., for identifying SNP's, or identifying specific alleles of 47476, 67210, 49875, 46842, 33201, 83378, 84233, 64708, 85041, or 84234. In a preferred embodiment, each oligonucleotide of the set has a different nucleotide at an interrogation position. In one embodiment, the set includes two oligonucleotides, each complementary to a different allele at a locus, e.g., a biallelic or polymorphic locus.

In another embodiment, the set includes four oligonucleotides, each having a different nucleotide (e.g., adenine, guanine, cytosine, or thymidine) at the interrogation position. The interrogation position can be a SNP or the site of a mutation. In another preferred embodiment, the oligonucleotides of the plurality are identical in sequence to one another (except for differences in length). The oligonucleotides can be provided with differential labels, such that an oligonucleotide that hybridizes to one allele provides a signal that is distinguishable from an oligonucleotide that hybridizes to a second allele. In still another embodiment, at least one of the oligonucleotides of the set has a nucleotide change at a position in addition to a query position, e.g., a destabilizing mutation to decrease the Tₘ of the oligonucleotide. In another embodiment, at least one oligonucleotide of the set has a non-natural nucleotide, e.g., inosine. In a preferred embodiment, the oligonucleotides are attached to a solid support, e.g., to different addresses of an array or to different beads or nanoparticles.

In a preferred embodiment the set of oligo nucleotides can be used to specifically amplify, e.g., by PCR, or detect, a 47476, 67210, 49875, 46842, 33201, 83378, 84233, 64708, 85041, or 84234 nucleic acid.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving a 47476, 67210, 49875, 46842, 33201, 83378, 84233, 64708, 85041, or 84234 gene.

Use of 47476, 67210, 49875, 46842, 33201, 83378, 84233, 64708, 85041, or 84234 Molecules as Surrogate Markers

The 47476, 67210, 49875, 46842, 33201, 83378, 84233, 64708, 85041, or 84234 molecules of the invention are also useful as markers of disorders or disease states, as markers for precursors of disease states, as markers for predisposition of disease states, as markers of drug activity, or as markers of the pharmacogenomic profile of a subject. Using the methods described herein, the presence, absence and/or quantity of the 47476, 67210, 49875, 46842, 33201, 83378, 84233, 64708, 85041, or 84234 molecules of the invention may be detected, and may be correlated with one or more biological states in vivo. For example, the 47476, 67210, 49875, 46842, 33201, 83378, 84233, 64708, 85041, or 84234 molecules of the invention may serve as surrogate markers for one or more disorders or disease states or for conditions leading up to disease states. As used herein, a “surrogate marker” is an objective biochemical marker which correlates with the absence or presence of a disease or disorder, or with the progression of a disease or disorder (e.g., with the presence or absence of a tumor). The presence or quantity of such markers is independent of the disease. Therefore, these markers may serve to indicate whether a particular course of treatment is effective in lessening a disease state or disorder. Surrogate markers are of particular use when the presence or extent of a disease state or disorder is difficult to assess through standard methodologies (e.g., early stage tumors), or when an assessment of disease progression is desired before a potentially dangerous clinical endpoint is reached (e.g., an assessment of cardiovascular disease may be made using cholesterol levels as a surrogate marker, and an analysis of HIV infection may be made using HIV RNA levels as a surrogate marker, well in advance of the undesirable clinical outcomes of myocardial infarction or fully-developed AIDS). Examples of the use of surrogate markers in the art include: Koomen et al. (2000) J. Mass. Spectrom. 35: 258-264; and James (1994) AIDS Treatment News Archive 209.

The 47476, 67210, 49875, 46842, 33201, 83378, 84233, 64708, 85041, or 84234 molecules of the invention are also useful as pharmacodynamic markers. As used herein, a “pharmacodynamic marker” is an objective biochemical marker which correlates specifically with drug effects. The presence or quantity of a pharmacodynamic marker is not related to the disease state or disorder for which the drug is being administered; therefore, the presence or quantity of the marker is indicative of the presence or activity of the drug in a subject. For example, a pharmacodynamic marker may be indicative of the concentration of the drug in a biological tissue, in that the marker is either expressed or transcribed or not expressed or transcribed in that tissue in relationship to the level of the drug. In this fashion, the distribution or uptake of the drug may be monitored by the pharmacodynamic marker. Similarly, the presence or quantity of the pharmacodynamic marker may be related to the presence or quantity of the metabolic product of a drug, such that the presence or quantity of the marker is indicative of the relative breakdown rate of the drug in vivo. Pharmacodynamic markers are of particular use in increasing the sensitivity of detection of drug effects, particularly when the drug is administered in low doses. Since even a small amount of a drug may be sufficient to activate multiple rounds of marker (e.g., a 47476, 67210, 49875, 46842, 33201, 83378, 84233, 64708, 85041, or 84234 marker) transcription or expression, the amplified marker may be in a quantity which is more readily detectable than the drug itself. Also, the marker may be more easily detected due to the nature of the marker itself; for example, using the methods described herein, anti-47476, 67210, 49875, 46842, 33201, 83378, 84233, 64708, 85041, or 84234 antibodies may be employed in an immune-based detection system for a 47476, 67210, 49875, 46842, 33201, 83378, 84233, 64708, 85041, or 84234 protein marker, or 47476, 67210, 49875, 46842, 33201, 83378, 84233, 64708, 85041, or 84234-specific radiolabeled probes may be used to detect a 47476, 67210, 49875, 46842, 33201, 83378, 84233, 64708, 85041, or 84234 mRNA marker. Furthermore, the use of a pharmacodynamic marker may offer mechanism-based prediction of risk due to drug treatment beyond the range of possible direct observations. Examples of the use of pharmacodynamic markers in the art include: Matsuda et al. U.S. Pat. No. 6,033,862; Hattis et al. (1991) Env. Health Perspect. 90: 229-238; Schentag (1999) Am. J. Health-Syst. Pharm. 56 Suppl. 3: S21-S24; and Nicolau (1999) Am, J. Health-Syst. Pharm. 56 Suppl. 3: S16-S20.

The 47476, 67210, 49875, 46842, 33201, 83378, 84233, 64708, 85041, or 84234 molecules of the invention are also useful as pharmacogenomic markers. As used herein, a “pharmacogenomic marker” is an objective biochemical marker which correlates with a specific clinical drug response or susceptibility in a subject (see, e.g., McLeod et al. (1999) Eur. J. Cancer 35:1650-1652). The presence or quantity of the pharmacogenomic marker is related to the predicted response of the subject to a specific drug or class of drugs prior to administration of the drug. By assessing the presence or quantity of one or more pharmacogenomic markers in a subject, a drug therapy which is most appropriate for the subject, or which is predicted to have a greater degree of success, may be selected. For example, based on the presence or quantity of RNA, or protein (e.g., 47476, 67210, 49875, 46842, 33201, 83378, 84233, 64708, 85041, or 84234 protein or RNA) for specific tumor markers in a subject, a drug or course of treatment may be selected that is optimized for the treatment of the specific tumor likely to be present in the subject. Similarly, the presence or absence of a specific sequence mutation in 47476, 67210, 49875, 46842, 33201, 83378, 84233, 64708, 85041, or 84234 DNA may correlate 47476, 67210, 49875, 46842, 33201, 83378, 84233, 64708, 85041, or 84234 drug response. The use of pharmacogenomic markers therefore permits the application of the most appropriate treatment for each subject without having to administer the therapy.

Pharmaceutical Compositions of 47476, 67210, 49875, 46842, 33201, 83378, 84233, 64708, 85041, and 84234

The nucleic acid and polypeptides, fragments thereof, as well as anti-47476, 67210, 49875, 46842, 33201, 83378, 84233, 64708, 85041, or 84234 antibodies (also referred to herein as “active compounds”) of the invention can be incorporated into pharmaceutical compositions. Such compositions typically include the nucleic acid molecule, protein, or antibody and a pharmaceutically acceptable carrier. As used herein the language “pharmaceutically acceptable carrier” includes solvents, dispersion media, coatings, antibacterial and antifungal agents, isotonic and absorption delaying agents, and the like, compatible with pharmaceutical administration. Supplementary active compounds can also be incorporated into the compositions.

A pharmaceutical composition is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor ELTM (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

As defined herein, a therapeutically effective amount of protein or polypeptide (i.e., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The protein or polypeptide can be administered one time per week for between about 1 to 10 weeks, preferably between 2 to 8 weeks, more preferably between about 3 to 7 weeks, and even more preferably for about 4, 5, or 6 weeks. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of a protein, polypeptide, or antibody can include a single treatment or, preferably, can include a series of treatments.

For antibodies, the preferred dosage is 0.1 mg/kg of body weight (generally 10 mg/kg to 20 mg/kg). If the antibody is to act in the brain, a dosage of 50 mg/kg to 100 mg/kg is usually appropriate. Generally, partially human antibodies and fully human antibodies have a longer half-life within the human body than other antibodies. Accordingly, lower dosages and less frequent administration is often possible. Modifications such as lipidation can be used to stabilize antibodies and to enhance uptake and tissue penetration (e.g., into the brain). A method for lipidation of antibodies is described by Cruikshank et al. ((1997) J. Acquired Immune Deficiency Syndromes and Human Retrovirology 14:193).

The present invention encompasses agents which modulate expression or activity. An agent may, for example, be a small molecule. For example, such small molecules include, but are not limited to, peptides, peptidomimetics (e.g., peptoids), amino acids, amino acid analogs, polynucleotides, polynucleotide analogs, nucleotides, nucleotide analogs, organic or inorganic compounds (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

Exemplary doses include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram. It is furthermore understood that appropriate doses of a small molecule depend upon the potency of the small molecule with respect to the expression or activity to be modulated. When one or more of these small molecules is to be administered to an animal (e.g., a human) in order to modulate expression or activity of a polypeptide or nucleic acid of the invention, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

An antibody (or fragment thereof) may be conjugated to a therapeutic moiety such as a cytotoxin, a therapeutic agent or a radioactive ion. A cytotoxin or cytotoxic agent includes any agent that is detrimental to cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, puromycin, maytansinoids, e.g., maytansinol (see U.S. Pat. No. 5,208,020), CC-1065 (see U.S. Pat. Nos. 5,475,092, 5,585,499, 5,846,545) and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, CC-1065, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine, vinblastine, taxol and maytansinoids). Radioactive ions include, but are not limited to iodine, yttrium and praseodymium.

The conjugates of the invention can be used for modifying a given biological response, the drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, α-interferon, β-interferon, nerve growth factor, platelet derived growth factor, tissue plasminogen activator; or, biological response modifiers such as, for example, lymphokines, interleukin-1 (“IL-1”), interleukin-2 (“IL-2”), interleukin-6 (“IL-6”), granulocyte macrophase colony stimulating factor (“GM-CSF”), granulocyte colony stimulating factor (“G-CSF”), or other growth factors. Alternatively, an antibody can be conjugated to a second antibody to form an antibody heteroconjugate as described by Segal in U.S. Pat. No. 4,676,980.

The nucleic acid molecules of the invention can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

The pharmaceutical compositions can be included in a container, pack, or dispenser together with instructions for administration.

Methods of Treatment for 47476, 67210, 49875, 46842, 33201, 83378, 84233, 64708, 85041, and 84234

The present invention provides for both prophylactic and therapeutic methods of treating a subject at risk of (or susceptible to) a disorder or having a disorder associated with aberrant or unwanted 47476, 67210, 49875, 46842, 33201, 83378, 84233, 64708, 85041, or 84234 expression or activity. As used herein, the term “treatment” is defined as the application or administration of a therapeutic agent to a patient, or application or administration of a therapeutic agent to an isolated tissue or cell line from a patient, who has a disease, a symptom of disease or a predisposition toward a disease, with the purpose to cure, heal, alleviate, relieve, alter, remedy, ameliorate, improve or affect the disease, the symptoms of disease or the predisposition toward disease. A therapeutic agent includes, but is not limited to, small molecules, peptides, antibodies, ribozymes and antisense oligonucleotides.

With regards to both prophylactic and therapeutic methods of treatment, such treatments may be specifically tailored or modified, based on knowledge obtained from the field of pharmacogenomics. “Pharmacogenomics”, as used herein, refers to the application of genomics technologies such as gene sequencing, statistical genetics, and gene expression analysis to drugs in clinical development and on the market. More specifically, the term refers the study of how a patient's genes determine his or her response to a drug (e.g., a patient's “drug response phenotype”, or “drug response genotype”.) Thus, another aspect of the invention provides methods for tailoring an individual's prophylactic or therapeutic treatment with either the 47476, 67210, 49875, 46842, 33201, 83378, 84233, 64708, 85041, or 84234 molecules of the present invention or 47476, 67210, 49875, 46842, 33201, 83378, 84233, 64708, 85041, or 84234 modulators according to that individual's drug response genotype. Pharmacogenomics allows a clinician or physician to target prophylactic or therapeutic treatments to patients who will most benefit from the treatment and to avoid treatment of patients who will experience toxic drug-related side effects.

In one aspect, the invention provides a method for preventing in a subject, a disease or condition associated with an aberrant or unwanted 47476, 67210, 49875, 46842, 33201, 83378, 84233, 64708, 85041, or 84234 expression or activity, by administering to the subject a 47476, 67210, 49875, 46842, 33201, 83378, 84233, 64708, 85041, or 84234 or an agent which modulates 47476, 67210, 49875, 46842, 33201, 83378, 84233, 64708, 85041, or 84234 expression or at least one 47476, 67210, 49875, 46842, 33201, 83378, 84233, 64708, 85041, or 84234 activity. Subjects at risk for a disease which is caused or contributed to by aberrant or unwanted 47476, 67210, 49875, 46842, 33201, 83378, 84233, 64708, 85041, or 84234 expression or activity can be identified by, for example, any or a combination of diagnostic or prognostic assays as described herein. Administration of a prophylactic agent can occur prior to the manifestation of symptoms characteristic of the 47476, 67210, 49875, 46842, 33201, 83378, 84233, 64708, 85041, or 84234 aberrance, such that a disease or disorder is prevented or, alternatively, delayed in its progression. Depending on the type of 47476, 67210, 49875, 46842, 33201, 83378, 84233, 64708, 85041, or 84234 aberrance, for example, a 47476, 67210, 49875, 46842, 33201, 83378, 84233, 64708, 85041, or 84234, 47476, 67210, 49875, 46842, 33201, 83378, 84233, 64708, 85041, or 84234 agonist or 47476, 67210, 49875, 46842, 33201, 83378, 84233, 64708, 85041, or 84234 antagonist agent can be used for treating the subject. The appropriate agent can be determined based on screening assays described herein.

It is possible that some 47476, 67210, 49875, 46842, 33201, 83378, 84233, 64708, 85041, or 84234 disorders can be caused, at least in part, by an abnormal level of gene product, or by the presence of a gene product exhibiting abnormal activity. As such, the reduction in the level and/or activity of such gene products would bring about the amelioration of disorder symptoms.

The 47476, 67210, 49875, 46842, 33201, 83378, 84233, 64708, 85041, or 84234 molecules can act as novel diagnostic targets and therapeutic agents for controlling one or more of cellular proliferative and/or differentiative disorders, immunological disorders (e.g., inflammatory disorders), red blood cell disorders, viral diseases, neurological disorders (e.g., brain disorders), pain or metabolic disorders, liver disorders, kidney disorders, disorders of the small intestine, disorder of metal ion imbalance, protein trafficking disorders, cardiovascular disorders, and disorders associated with bone metabolism, as discussed above.

Successful treatment of 47476, 67210, 49875, 46842, 33201, 83378, 84233, 64708, 85041, or 84234 disorders can be brought about by techniques that serve to inhibit the expression or activity of target gene products. For example, compounds, e.g., an agent identified using an assays described above, that proves to exhibit negative modulatory activity, can be used in accordance with the invention to prevent and/or ameliorate symptoms of 47476, 67210, 49875, 46842, 33201, 83378, 84233, 64708, 85041, or 84234 disorders. Such molecules can include, but are not limited to peptides, phosphopeptides, small organic or inorganic molecules, or antibodies (including, for example, polyclonal, monoclonal, humanized, anti-idiotypic, chimeric or single chain antibodies, and Fab, F(ab′)₂ and Fab expression library fragments, scFV molecules, and epitope-binding fragments thereof).

Further, antisense and ribozyme molecules that inhibit expression of the target gene can also be used in accordance with the invention to reduce the level of target gene expression, thus effectively reducing the level of target gene activity. Still further, triple helix molecules can be utilized in reducing the level of target gene activity. Antisense, ribozyme and triple helix molecules are discussed above.

It is possible that the use of antisense, ribozyme, and/or triple helix molecules to reduce or inhibit mutant gene expression can also reduce or inhibit the transcription (triple helix) and/or translation (antisense, ribozyme) of mRNA produced by normal target gene alleles, such that the concentration of normal target gene product present can be lower than is necessary for a normal phenotype. In such cases, nucleic acid molecules that encode and express target gene polypeptides exhibiting normal target gene activity can be introduced into cells via gene therapy method. Alternatively, in instances in that the target gene encodes an extracellular protein, it can be preferable to co-administer normal target gene protein into the cell or tissue in order to maintain the requisite level of cellular or tissue target gene activity.

Another method by which nucleic acid molecules may be utilized in treating or preventing a disease characterized by 47476, 67210, 49875, 46842, 33201, 83378, 84233, 64708, 85041, or 84234 expression is through the use of aptamer molecules specific for 47476, 67210, 49875, 46842, 33201, 83378, 84233, 64708, 85041, or 84234 protein. Aptamers are nucleic acid molecules having a tertiary structure which permits them to specifically bind to protein ligands (see, e.g., Osborne, et al. (1997) Curr. Opin. Chem Biol. 1: 5-9; and Patel, D. J. (1997) Curr Opin Chem Biol 1:32-46). Since nucleic acid molecules may in many cases be more conveniently introduced into target cells than therapeutic protein molecules may be, aptamers offer a method by which 47476, 67210, 49875, 46842, 33201, 83378, 84233, 64708, 85041, or 84234 protein activity may be specifically decreased without the introduction of drugs or other molecules which may have pluripotent effects.

Antibodies can be generated that are both specific for target gene product and that reduce target gene product activity. Such antibodies may, therefore, by administered in instances whereby negative modulatory techniques are appropriate for the treatment of 47476, 67210, 49875, 46842, 33201, 83378, 84233, 64708, 85041, or 84234 disorders. For a description of antibodies, see the Antibody section above.

In circumstances wherein injection of an animal or a human subject with a 47476, 67210, 49875, 46842, 33201, 83378, 84233, 64708, 85041, or 84234 protein or epitope for stimulating antibody production is harmful to the subject, it is possible to generate an immune response against 47476, 67210, 49875, 46842, 33201, 83378, 84233, 64708, 85041, or 84234 through the use of anti-idiotypic antibodies (see, for example, Herlyn, D. (1999) Ann Med 31:66-78; and Bhattacharya-Chatterjee, M., and Foon, K. A. (1998) Cancer Treat Res. 94:51-68). If an anti-idiotypic antibody is introduced into a mammal or human subject, it should stimulate the production of anti-anti-idiotypic antibodies, which should be specific to the 47476, 67210, 49875, 46842, 33201, 83378, 84233, 64708, 85041, or 84234 protein. Vaccines directed to a disease characterized by 47476, 67210, 49875, 46842, 33201, 83378, 84233, 64708, 85041, or 84234 expression may also be generated in this fashion.

In instances where the target antigen is intracellular and whole antibodies are used, internalizing antibodies may be preferred. Lipofectin or liposomes can be used to deliver the antibody or a fragment of the Fab region that binds to the target antigen into cells. Where fragments of the antibody are used, the smallest inhibitory fragment that binds to the target antigen is preferred. For example, peptides having an amino acid sequence corresponding to the Fv region of the antibody can be used. Alternatively, single chain neutralizing antibodies that bind to intracellular target antigens can also be administered. Such single chain antibodies can be administered, for example, by expressing nucleotide sequences encoding single-chain antibodies within the target cell population (see e.g., Marasco et al. (1993) Proc. Natl. Acad. Sci. USA 90:7889-7893).

The identified compounds that inhibit target gene expression, synthesis and/or activity can be administered to a patient at therapeutically effective doses to prevent, treat or ameliorate 47476, 67210, 49875, 46842, 33201, 83378, 84233, 64708, 85041, or 84234 disorders. A therapeutically effective dose refers to that amount of the compound sufficient to result in amelioration of symptoms of the disorders. Toxicity and therapeutic efficacy of such compounds can be determined by standard pharmaceutical procedures as described above.

The data obtained from the cell culture assays and animal studies can be used in formulating a range of dosage for use in humans. The dosage of such compounds lies preferably within a range of circulating concentrations that include the ED₅₀ with little or no toxicity. The dosage can vary within this range depending upon the dosage form employed and the route of administration utilized. For any compound used in the method of the invention, the therapeutically effective dose can be estimated initially from cell culture assays. A dose can be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma can be measured, for example, by high performance liquid chromatography.

Another example of determination of effective dose for an individual is the ability to directly assay levels of “free” and “bound” compound in the serum of the test subject. Such assays may utilize antibody mimics and/or “biosensors” that have been created through molecular imprinting techniques. The compound which is able to modulate 47476, 67210, 49875, 46842, 33201, 83378, 84233, 64708, 85041, or 84234 activity is used as a template, or “imprinting molecule”, to spatially organize polymerizable monomers prior to their polymerization with catalytic reagents. The subsequent removal of the imprinted molecule leaves a polymer matrix which contains a repeated “negative image” of the compound and is able to selectively rebind the molecule under biological assay conditions. A detailed review of this technique can be seen in Ansell, R. J. et al. (1996) Current Opinion in Biotechnology 7:89-94 and in Shea, K. J. (1994) Trends in Polymer Science 2:166-173. Such “imprinted” affinity matrixes are amenable to ligand-binding assays, whereby the immobilized monoclonal antibody component is replaced by an appropriately imprinted matrix. An example of the use of such matrixes in this way can be seen in Vlatakis, G. et al. (1993) Nature 361:645-647. Through the use of isotope-labeling, the “free” concentration of compound which modulates the expression or activity of 47476, 67210, 49875, 46842, 33201, 83378, 84233, 64708, 85041, or 84234 can be readily monitored and used in calculations of IC₅₀.

Such “imprinted” affinity matrixes can also be designed to include fluorescent groups whose photon-emitting properties measurably change upon local and selective binding of target compound. These changes can be readily assayed in real time using appropriate fiberoptic devices, in turn allowing the dose in a test subject to be quickly optimized based on its individual IC₅₀. An rudimentary example of such a “biosensor” is discussed in Kriz, D. et al. (1995) Analytical Chemistry 67:2142-2144.

Another aspect of the invention pertains to methods of modulating 47476, 67210, 49875, 46842, 33201, 83378, 84233, 64708, 85041, or 84234 expression or activity for therapeutic purposes. Accordingly, in an exemplary embodiment, the modulatory method of the invention involves contacting a cell with a 47476, 67210, 49875, 46842, 33201, 83378, 84233, 64708, 85041, or 84234 or agent that modulates one or more of the activities of 47476, 67210, 49875, 46842, 33201, 83378, 84233, 64708, 85041, or 84234 protein activity associated with the cell. An agent that modulates 47476, 67210, 49875, 46842, 33201, 83378, 84233, 64708, 85041, or 84234 protein activity can be an agent as described herein, such as a nucleic acid or a protein, a naturally-occurring target molecule of a 47476, 67210, 49875, 46842, 33201, 83378, 84233, 64708, 85041, or 84234 protein (e.g., a 47476, 67210, 49875, 46842, 33201, 83378, 84233, 64708, 85041, or 84234 substrate or receptor), a 47476, 67210, 49875, 46842, 33201, 83378, 84233, 64708, 85041, or 84234 antibody, a 47476, 67210, 49875, 46842, 33201, 83378, 84233, 64708, 85041, or 84234 agonist or antagonist, a peptidomimetic of a 47476, 67210, 49875, 46842, 33201, 83378, 84233, 64708, 85041, or 84234 agonist or antagonist, or other small molecule.

In one embodiment, the agent stimulates one or 47476, 67210, 49875, 46842, 33201, 83378, 84233, 64708, 85041, or 84234 activities. Examples of such stimulatory agents include active 47476, 67210, 49875, 46842, 33201, 83378, 84233, 64708, 85041, or 84234 protein and a nucleic acid molecule encoding 47476, 67210, 49875, 46842, 33201, 83378, 84233, 64708, 85041, or 84234. In another embodiment, the agent inhibits one or more 47476, 67210, 49875, 46842, 33201, 83378, 84233, 64708, 85041, or 84234 activities. Examples of such inhibitory agents include antisense 47476, 67210, 49875, 46842, 33201, 83378, 84233, 64708, 85041, or 84234 nucleic acid molecules, anti-47476, 67210, 49875, 46842, 33201, 83378, 84233, 64708, 85041, or 84234 antibodies, and 47476, 67210, 49875, 46842, 33201, 83378, 84233, 64708, 85041, or 84234 inhibitors. These modulatory methods can be performed in vitro (e.g., by culturing the cell with the agent) or, alternatively, in vivo (e.g., by administering the agent to a subject). As such, the present invention provides methods of treating an individual afflicted with a disease or disorder characterized by aberrant or unwanted expression or activity of a 47476, 67210, 49875, 46842, 33201, 83378, 84233, 64708, 85041, or 84234 protein or nucleic acid molecule. In one embodiment, the method involves administering an agent (e.g., an agent identified by a screening assay described herein), or combination of agents that modulates (e.g., up regulates or down regulates) 47476, 67210, 49875, 46842, 33201, 83378, 84233, 64708, 85041, or 84234 expression or activity. In another embodiment, the method involves administering a 47476, 67210, 49875, 46842, 33201, 83378, 84233, 64708, 85041, or 84234 protein or nucleic acid molecule as therapy to compensate for reduced, aberrant, or unwanted 47476, 67210, 49875, 46842, 33201, 83378, 84233, 64708, 85041, or 84234 expression or activity.

Stimulation of 47476, 67210, 49875, 46842, 33201, 83378, 84233, 64708, 85041, or 84234 activity is desirable in situations in which 47476, 67210, 49875, 46842, 33201, 83378, 84233, 64708, 85041, or 84234 is abnormally downregulated and/or in which increased 47476, 67210, 49875, 46842, 33201, 83378, 84233, 64708, 85041, or 84234 activity is likely to have a beneficial effect. For example, stimulation of 47476, 67210, 49875, 46842, 33201, 83378, 84233, 64708, 85041, or 84234 activity is desirable in situations in which a 47476, 67210, 49875, 46842, 33201, 83378, 84233, 64708, 85041, or 84234 is downregulated and/or in which increased 47476, 67210, 49875, 46842, 33201, 83378, 84233, 64708, 85041, or 84234 activity is likely to have a beneficial effect. Likewise, inhibition of 47476, 67210, 49875, 46842, 33201, 83378, 84233, 64708, 85041, or 84234 activity is desirable in situations in which 47476, 67210, 49875, 46842, 33201, 83378, 84233, 64708, 85041, or 84234 is abnormally upregulated and/or in which decreased 47476, 67210, 49875, 46842, 33201, 83378, 84233, 64708, 85041, or 84234 activity is likely to have a beneficial effect.

47476, 67210, 49875, 46842, 33201, 83378, 84233, 64708, 85041, and 84234 Pharmacogenomics

The 47476, 67210, 49875, 46842, 33201, 83378, 84233, 64708, 85041, or 84234 molecules of the present invention, as well as agents, or modulators which have a stimulatory or inhibitory effect on 47476, 67210, 49875, 46842, 33201, 83378, 84233, 64708, 85041, or 84234 activity (e.g., 47476, 67210, 49875, 46842, 33201, 83378, 84233, 64708, 85041, or 84234 gene expression) as identified by a screening assay described herein can be administered to individuals to treat (prophylactically or therapeutically) 47476, 67210, 49875, 46842, 33201, 83378, 84233, 64708, 85041, or 84234 associated disorders (e.g., immunological disorders, neurological disorders, metabolic disorders, cellular proliferation and/or differentiation disorders, disorders of metal ion imbalance, protein trafficing disorders, or cardiovascular disorders) associated with aberrant or unwanted 47476, 67210, 49875, 46842, 33201, 83378, 84233, 64708, 85041, or 84234 activity. In conjunction with such treatment, pharmacogenomics (i.e., the study of the relationship between an individual's genotype and that individual's response to a foreign compound or drug) may be considered. Differences in metabolism of therapeutics can lead to severe toxicity or therapeutic failure by altering the relation between dose and blood concentration of the pharmacologically active drug. Thus, a physician or clinician may consider applying knowledge obtained in relevant pharmacogenomics studies in determining whether to administer a 47476, 67210, 49875, 46842, 33201, 83378, 84233, 64708, 85041, or 84234 molecule or 47476, 67210, 49875, 46842, 33201, 83378, 84233, 64708, 85041, or 84234 modulator as well as tailoring the dosage and/or therapeutic regimen of treatment with a 47476, 67210, 49875, 46842, 33201, 83378, 84233, 64708, 85041, or 84234 molecule or 47476, 67210, 49875, 46842, 33201, 83378, 84233, 64708, 85041, or 84234 modulator.

Pharmacogenomics deals with clinically significant hereditary variations in the response to drugs due to altered drug disposition and abnormal action in affected persons. See, for example, Eichelbaum, M. et al. (1996) Clin. Exp. Pharmacol. Physiol. 23:983-985 and Linder, M. W. et al. (1997) Clin. Chem. 43:254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare genetic defects or as naturally-occurring polymorphisms. For example, glucose-6-phosphate dehydrogenase deficiency (G6PD) is a common inherited enzymopathy in which the main clinical complication is haemolysis after ingestion of oxidant drugs (anti-malarials, sulfonamides, analgesics, nitrofurans) and consumption of fava beans.

One pharmacogenomics approach to identifying genes that predict drug response, known as “a genome-wide association”, relies primarily on a high-resolution map of the human genome consisting of already known gene-related markers (e.g., a “bi-allelic” gene marker map which consists of 60,000-100,000 polymorphic or variable sites on the human genome, each of which has two variants.) Such a high-resolution genetic map can be compared to a map of the genome of each of a statistically significant number of patients taking part in a Phase II/III drug trial to identify markers associated with a particular observed drug response or side effect. Alternatively, such a high resolution map can be generated from a combination of some ten-million known single nucleotide polymorphisms (SNPs) in the human genome. As used herein, a “SNP” is a common alteration that occurs in a single nucleotide base in a stretch of DNA. For example, a SNP may occur once per every 1000 bases of DNA. A SNP may be involved in a disease process, however, the vast majority may not be disease-associated. Given a genetic map based on the occurrence of such SNPs, individuals can be grouped into genetic categories depending on a particular pattern of SNPs in their individual genome. In such a manner, treatment regimens can be tailored to groups of genetically similar individuals, taking into account traits that may be common among such genetically similar individuals.

Alternatively, a method termed the “candidate gene approach,” can be utilized to identify genes that predict drug response. According to this method, if a gene that encodes a drug's target is known (e.g., a 47476, 67210, 49875, 46842, 33201, 83378, 84233, 64708, 85041, or 84234 protein of the present invention), all common variants of that gene can be fairly easily identified in the population and it can be determined if having one version of the gene versus another is associated with a particular drug response.

Alternatively, a method termed the “gene expression profiling,” can be utilized to identify genes that predict drug response. For example, the gene expression of an animal dosed with a drug (e.g., a 47476, 67210, 49875, 46842, 33201, 83378, 84233, 64708, 85041, or 84234 molecule or 47476, 67210, 49875, 46842, 33201, 83378, 84233, 64708, 85041, or 84234 modulator of the present invention) can give an indication whether gene pathways related to toxicity have been turned on.

Information generated from more than one of the above pharmacogenomics approaches can be used to determine appropriate dosage and treatment regimens for prophylactic or therapeutic treatment of an individual. This knowledge, when applied to dosing or drug selection, can avoid adverse reactions or therapeutic failure and thus enhance therapeutic or prophylactic efficiency when treating a subject with a 47476, 67210, 49875, 46842, 33201, 83378, 84233, 64708, 85041, or 84234 molecule or 47476, 67210, 49875, 46842, 33201, 83378, 84233, 64708, 85041, or 84234 modulator, such as a modulator identified by one of the exemplary screening assays described herein.

The present invention further provides methods for identifying new agents, or combinations, that are based on identifying agents that modulate the activity of one or more of the gene products encoded by one or more of the 47476, 67210, 49875, 46842, 33201, 83378, 84233, 64708, 85041, or 84234 genes of the present invention, wherein these products may be associated with resistance of the cells to a therapeutic agent. Specifically, the activity of the proteins encoded by the 47476, 67210, 49875, 46842, 33201, 83378, 84233, 64708, 85041, or 84234 genes of the present invention can be used as a basis for identifying agents for overcoming agent resistance. By blocking the activity of one or more of the resistance proteins, target cells, e.g., human cells, will become sensitive to treatment with an agent that the unmodified target cells were resistant to.

Monitoring the influence of agents (e.g., drugs) on the expression or activity of a 47476, 67210, 49875, 46842, 33201, 83378, 84233, 64708, 85041, or 84234 protein can be applied in clinical trials. For example, the effectiveness of an agent determined by a screening assay as described herein to increase 47476, 67210, 49875, 46842, 33201, 83378, 84233, 64708, 85041, or 84234 gene expression, protein levels, or upregulate 47476, 67210, 49875, 46842, 33201, 83378, 84233, 64708, 85041, or 84234 activity, can be monitored in clinical trials of subjects exhibiting decreased 47476, 67210, 49875, 46842, 33201, 83378, 84233, 64708, 85041, or 84234 gene expression, protein levels, or downregulated 47476, 67210, 49875, 46842, 33201, 83378, 84233, 64708, 85041, or 84234 activity. Alternatively, the effectiveness of an agent determined by a screening assay to decrease 47476, 67210, 49875, 46842, 33201, 83378, 84233, 64708, 85041, or 84234 gene expression, protein levels, or downregulate 47476, 67210, 49875, 46842, 33201, 83378, 84233, 64708, 85041, or 84234 activity, can be monitored in clinical trials of subjects exhibiting increased 47476, 67210, 49875, 46842, 33201, 83378, 84233, 64708, 85041, or 84234 gene expression, protein levels, or upregulated 47476, 67210, 49875, 46842, 33201, 83378, 84233, 64708, 85041, or 84234 activity. In such clinical trials, the expression or activity of a 47476, 67210, 49875, 46842, 33201, 83378, 84233, 64708, 85041, or 84234 gene, and preferably, other genes that have been implicated in, for example, a 47476, 67210, 49875, 46842, 33201, 83378, 84233, 64708, 85041, or 84234-associated disorder can be used as a “read out” or markers of the phenotype of a particular cell.

47476, 67210, 49875, 46842, 33201, 83378, 84233, 64708, 85041, or 84234 Informatics

The sequence of a 47476, 67210, 49875, 46842, 33201, 83378, 84233, 64708, 85041, or 84234 molecule is provided in a variety of media to facilitate use thereof. A sequence can be provided as a manufacture, other than an isolated nucleic acid or amino acid molecule, which contains a 47476, 67210, 49875, 46842, 33201, 83378, 84233, 64708, 85041, or 84234. Such a manufacture can provide a nucleotide or amino acid sequence, e.g., an open reading frame, in a form which allows examination of the manufacture using means not directly applicable to examining the nucleotide or amino acid sequences, or a subset thereof, as they exists in nature or in purified form. The sequence information can include, but is not limited to, 47476, 67210, 49875, 46842, 33201, 83378, 84233, 64708, 85041, or 84234 full-length nucleotide and/or amino acid sequences, partial nucleotide and/or amino acid sequences, polymorphic sequences including single nucleotide polymorphisms (SNPs), epitope sequence, and the like. In a preferred embodiment, the manufacture is a machine-readable medium, e.g., a magnetic, optical, chemical or mechanical information storage device.

As used herein, “machine-readable media” refers to any medium that can be read and accessed directly by a machine, e.g., a digital computer or analogue computer. Non-limiting examples of a computer include a desktop PC, laptop, mainframe, server (e.g., a web server, network server, or server farm), handheld digital assistant, pager, mobile telephone, and the like. The computer can be stand-alone or connected to a communications network, e.g., a local area network (such as a VPN or intranet), a wide area network (e.g., an Extranet or the Internet), or a telephone network (e.g., a wireless, DSL, or ISDN network). Machine-readable media include, but are not limited to: magnetic storage media, such as floppy discs, hard disc storage medium, and magnetic tape; optical storage media such as CD-ROM; electrical storage media such as RAM, ROM, EPROM, EEPROM, flash memory, and the like; and hybrids of these categories such as magnetic/optical storage media.

A variety of data storage structures are available to a skilled artisan for creating a machine-readable medium having recorded thereon a nucleotide or amino acid sequence of the present invention. The choice of the data storage structure will generally be based on the means chosen to access the stored information. In addition, a variety of data processor programs and formats can be used to store the nucleotide sequence information of the present invention on computer readable medium. The sequence information can be represented in a word processing text file, formatted in commercially-available software such as WordPerfect and Microsoft Word, or represented in the form of an ASCII file, stored in a database application, such as DB2, Sybase, Oracle, or the like. The skilled artisan can readily adapt any number of data processor structuring formats (e.g., text file or database) in order to obtain computer readable medium having recorded thereon the nucleotide sequence information of the present invention.

In a preferred embodiment, the sequence information is stored in a relational database (such as Sybase or Oracle). The database can have a first table for storing sequence (nucleic acid and/or amino acid sequence) information. The sequence information can be stored in one field (e.g., a first column) of a table row and an identifier for the sequence can be store in another field (e.g., a second column) of the table row. The database can have a second table, e.g., storing annotations. The second table can have a field for the sequence identifier, a field for a descriptor or annotation text (e.g., the descriptor can refer to a functionality of the sequence, a field for the initial position in the sequence to which the annotation refers, and a field for the ultimate position in the sequence to which the annotation refers. Non-limiting examples for annotation to nucleic acid sequences include polymorphisms (e.g., SNP's) translational regulatory sites and splice junctions. Non-limiting examples for annotations to amino acid sequence include polypeptide domains, e.g., a domain described herein; active sites and other functional amino acids; and modification sites.

By providing the nucleotide or amino acid sequences of the invention in computer readable form, the skilled artisan can routinely access the sequence information for a variety of purposes. For example, one skilled in the art can use the nucleotide or amino acid sequences of the invention in computer readable form to compare a target sequence or target structural motif with the sequence information stored within the data storage means. A search is used to identify fragments or regions of the sequences of the invention which match a particular target sequence or target motif. The search can be a BLAST search or other routine sequence comparison, e.g., a search described herein.

Thus, in one aspect, the invention features a method of analyzing 47476, 67210, 49875, 46842, 33201, 83378, 84233, 64708, 85041, or 84234, e.g., analyzing structure, function, or relatedness to one or more other nucleic acid or amino acid sequences. The method includes: providing a 47476, 67210, 49875, 46842, 33201, 83378, 84233, 64708, 85041, or 84234 nucleic acid or amino acid sequence; comparing the 47476, 67210, 49875, 46842, 33201, 83378, 84233, 64708, 85041, or 84234 sequence with a second sequence, e.g., one or more preferably a plurality of sequences from a collection of sequences, e.g., a nucleic acid or protein sequence database to thereby analyze 47476, 67210, 49875, 46842, 33201, 83378, 84233, 64708, 85041, or 84234. The method can be performed in a machine, e.g., a computer, or manually by a skilled artisan.

The method can include evaluating the sequence identity between a 47476, 67210, 49875, 46842, 33201, 83378, 84233, 64708, 85041, or 84234 sequence and a database sequence. The method can be performed by accessing the database at a second site, e.g., over the Internet.

As used herein, a “target sequence” can be any DNA or amino acid sequence of six or more nucleotides or two or more amino acids. A skilled artisan can readily recognize that the longer a target sequence is, the less likely a target sequence will be present as a random occurrence in the database. Typical sequence lengths of a target sequence are from about 10 to 100 amino acids or from about 30 to 300 nucleotide residues. However, it is well recognized that commercially important fragments, such as sequence fragments involved in gene expression and protein processing, may be of shorter length.

Computer software is publicly available which allows a skilled artisan to access sequence information provided in a computer readable medium for analysis and comparison to other sequences. A variety of known algorithms are disclosed publicly and a variety of commercially available software for conducting search means are and can be used in the computer-based systems of the present invention. Examples of such software include, but are not limited to, MacPattern (EMBL), BLASTN and BLASTX (NCBI).

Thus, the invention features a method of making a computer readable record of a sequence of a 47476, 67210, 49875, 46842, 33201, 83378, 84233, 64708, 85041, or 84234 sequence which includes recording the sequence on a computer readable matrix. In a preferred embodiment the record includes one or more of the following: identification of an ORF; identification of a domain, region, or site; identification of the start of transcription; identification of the transcription terminator; the full length amino acid sequence of the protein, or a mature form thereof; the 5′ end of the translated region.

In another aspect, the invention features, a method of analyzing a sequence. The method includes: providing a 47476, 67210, 49875, 46842, 33201, 83378, 84233, 64708, 85041, or 84234 sequence, or record, in machine-readable form; comparing a second sequence to the 47476, 67210, 49875, 46842, 33201, 83378, 84233, 64708, 85041, or 84234 sequence; thereby analyzing a sequence. Comparison can include comparing to sequences for sequence identity or determining if one sequence is included within the other, e.g., determining if the 47476, 67210, 49875, 46842, 33201, 83378, 84233, 64708, 85041, or 84234 sequence includes a sequence being compared. In a preferred embodiment the 47476, 67210, 49875, 46842, 33201, 83378, 84233, 64708, 85041, or 84234 or second sequence is stored on a first computer, e.g., at a first site and the comparison is performed, read, or recorded on a second computer, e.g., at a second site. E.g., the 47476, 67210, 49875, 46842, 33201, 83378, 84233, 64708, 85041, or 84234 or second sequence can be stored in a public or proprietary database in one computer, and the results of the comparison performed, read, or recorded on a second computer. In a preferred embodiment the record includes one or more of the following: identification of an ORF; identification of a domain, region, or site; identification of the start of transcription; identification of the transcription terminator; the full length amino acid sequence of the protein, or a mature form thereof; the 5′ end of the translated region.

In another aspect, the invention provides a machine-readable medium for holding instructions for performing a method for determining whether a subject has a 47476, 67210, 49875, 46842, 33201, 83378, 84233, 64708, 85041, or 84234-associated disease or disorder or a pre-disposition to a 47476, 67210, 49875, 46842, 33201, 83378, 84233, 64708, 85041, or 84234-associated disease or disorder, wherein the method comprises the steps of determining 47476, 67210, 49875, 46842, 33201, 83378, 84233, 64708, 85041, or 84234 sequence information associated with the subject and based on the 47476, 67210, 49875, 46842, 33201, 83378, 84233, 64708, 85041, or 84234 sequence information, determining whether the subject has a 47476, 67210, 49875, 46842, 33201, 83378, 84233, 64708, 85041, or 84234-associated disease or disorder or a pre-disposition to a 47476, 67210, 49875, 46842, 33201, 83378, 84233, 64708, 85041, or 84234-associated disease or disorder and/or recommending a particular treatment for the disease, disorder or pre-disease condition.

The invention further provides in an electronic system and/or in a network, a method for determining whether a subject has a 47476, 67210, 49875, 46842, 33201, 83378, 84233, 64708, 85041, or 84234-associated disease or disorder or a pre-disposition to a disease associated with a 47476, 67210, 49875, 46842, 33201, 83378, 84233, 64708, 85041, or 84234 wherein the method comprises the steps of determining 47476, 67210, 49875, 46842, 33201, 83378, 84233, 64708, 85041, or 84234 sequence information associated with the subject, and based on the 47476, 67210, 49875, 46842, 33201, 83378, 84233, 64708, 85041, or 84234 sequence information, determining whether the subject has a 47476, 67210, 49875, 46842, 33201, 83378, 84233, 64708, 85041, or 84234-associated disease or disorder or a pre-disposition to a 47476, 67210, 49875, 46842, 33201, 83378, 84233, 64708, 85041, or 84234-associated disease or disorder, and/or recommending a particular treatment for the disease, disorder or pre-disease condition. In a preferred embodiment, the method further includes the step of receiving information, e.g., phenotypic or genotypic information, associated with the subject and/or acquiring from a network phenotypic information associated with the subject. The information can be stored in a database, e.g., a relational database. In another embodiment, the method further includes accessing the database, e.g., for records relating to other subjects, comparing the 47476, 67210, 49875, 46842, 33201, 83378, 84233, 64708, 85041, or 84234 sequence of the subject to the 47476, 67210, 49875, 46842, 33201, 83378, 84233, 64708, 85041, or 84234 sequences in the database to thereby determine whether the subject as a 47476, 67210, 49875, 46842, 33201, 83378, 84233, 64708, 85041, or 84234-associated disease or disorder, or a pre-disposition for such.

The present invention also provides in a network, a method for determining whether a subject has a 47476, 67210, 49875, 46842, 33201, 83378, 84233, 64708, 85041, or 84234 associated disease or disorder or a pre-disposition to a 47476, 67210, 49875, 46842, 33201, 83378, 84233, 64708, 85041, or 84234-associated disease or disorder associated with 47476, 67210, 49875, 46842, 33201, 83378, 84233, 64708, 85041, or 84234, said method comprising the steps of receiving 47476, 67210, 49875, 46842, 33201, 83378, 84233, 64708, 85041, or 84234 sequence information from the subject and/or information related thereto, receiving phenotypic information associated with the subject, acquiring information from the network corresponding to 47476, 67210, 49875, 46842, 33201, 83378, 84233, 64708, 85041, or 84234 and/or corresponding to a 47476, 67210, 49875, 46842, 33201, 83378, 84233, 64708, 85041, or 84234-associated disease or disorder (e.g., an immunological disorder, neurological disorder, metabolic disorder, cellular proliferation and/or differentiation disorder, disorder of metal ion imbalance, a protein trafficing disorder, or a cardiovascular disorder), and based on one or more of the phenotypic information, the 47476, 67210, 49875, 46842, 33201, 83378, 84233, 64708, 85041, or 84234 information (e.g., sequence information and/or information related thereto), and the acquired information, determining whether the subject has a 47476, 67210, 49875, 46842, 33201, 83378, 84233, 64708, 85041, or 84234-associated disease or disorder or a pre-disposition to a 47476, 67210, 49875, 46842, 33201, 83378, 84233, 64708, 85041, or 84234-associated disease or disorder. The method may further comprise the step of recommending a particular treatment for the disease, disorder or pre-disease condition.

The present invention also provides a method for determining whether a subject has a 47476, 67210, 49875, 46842, 33201, 83378, 84233, 64708, 85041, or 84234-associated disease or disorder or a pre-disposition to a 47476, 67210, 49875, 46842, 33201, 83378, 84233, 64708, 85041, or 84234-associated disease or disorder, said method comprising the steps of receiving information related to 47476, 67210, 49875, 46842, 33201, 83378, 84233, 64708, 85041, or 84234 (e.g., sequence information and/or information related thereto), receiving phenotypic information associated with the subject, acquiring information from the network related to 47476, 67210, 49875, 46842, 33201, 83378, 84233, 64708, 85041, or 84234 and/or related to a 47476, 67210, 49875, 46842, 33201, 83378, 84233, 64708, 85041, or 84234-associated disease or disorder, and based on one or more of the phenotypic information, the 47476, 67210, 49875, 46842, 33201, 83378, 84233, 64708, 85041, or 84234 information, and the acquired information, determining whether the subject has a 47476, 67210, 49875, 46842, 33201, 83378, 84233, 64708, 85041, or 84234-associated disease or disorder or a pre-disposition to a 47476, 67210, 49875, 46842, 33201, 83378, 84233, 64708, 85041, or 84234-associated disease or disorder. The method may further comprise the step of recommending a particular treatment for the disease, disorder or pre-disease condition.

This invention is further illustrated by the following examples that should not be construed as limiting. The contents of all references, patents and published patent applications cited throughout this application are incorporated herein by reference.

### BACKGROUND OF THE 21617 AND 55562 INVENTION

Dehydrogenases

Short chain dehydrogenases (SDRs) are a large and diverse collection of enzymes grouped into a superfamily comprising over 700 different enzymes including isomerases, lyases, and oxidoreductases (Opperman et al. (1999) Enzymology and Molecular Biology of Carbonyl Metabolism, 7 ed., Weiner et al., Plenum Publishers, NY p. 365-371). They are important in metabolism of small molecules, production/removal of biologically important molecules that modulate development and growth, elimination of toxins, and associated physiological processes and pathological conditions. The enzymes of this family cover a wide range of substrate specificities including sugars, steroids, alcohols, prostaglandins, metabolites (e.g., lipids), and aromatic compounds (Opperman et al. (1999), supra, p. 373-377).

Members of the alcohol dehydrogenase and short-chain dehydrogenase/reductase families catalyze the reversible, rate limiting conversion of retinol to retinal, while the oxidation of retinal to retinoic acid is catalyzed by members of the aldehyde dehydrogenase or P450 enzyme families (Deuster et al. (1996), Biochemistry 35:12221-12227). Other SDR/retinol dehydrogenases function in the visual cycle by converting either 11-cis-retinol to 11-cis-retinal or all trans-retinal to all trans-retinol (Simon et al. (1995) J Biol Chem 270:1107-1112). Retinoic acid plays a key role in the regulation of embryonic development, spermatogenesis, and epithelial differentiation (Chambon et al. (1996), FASEB J 10:940-954, and Mangelsdorf et al. (1995), Cell 83:841-850).

Alcohol dehydrogenases play fundamental roles in degradative, synthetic, and detoxification pathways and have been implicated in a variety of developmental processes and pathophysiological disease states. For example, allelic variations of ADH2 and ADH3 appear to influence the susceptibility to alcoholism and alcoholic liver cirrhosis in Asians (Thomasson et al. (1991), Am J Hum Genet 48:677-681, Chao et al. (1994), Hepatology 19:360-366, and Higuchi et al. (1995), Am J Psychiatry 152:1219-1221).

Tetratricopeptide Repeats

Tetratricopeptide repeats (TPR) are found in a diverse collection of polypeptides (Boebel and Yanagida (1991) Trends Biochem Sci. 16:173; Lamb et al. (1995) Trends Biochem. Sci. 20:257). Typically, each repeat folds as an anti-parallel pair of α-helices; adjacent repeats pack against each other to form an extensive accordion-like structure. This polypeptide fold can serve a variety of functions, including scaffolding protein-protein interactions for complex formation and regulation of protein function.

For example, the serine/threonine protein phosphatase PP5 has three tandem TPR motifs that have multiple functions (see, e.g., Das et al. (1998), EMBO J. 17:1192-99). In part, the TPR domain of PP5 is an allosteric regulator that inhibits phosphatase function until triggered by arachidonic acid. Arachidonic acid binds to the TPR domain, and relieves the inhibition, thereby activating the enzyme. Additionally, the TPR domain interacts with hsp90 and the kinase domain of the ANP-guanylate cyclase receptor in a signalling network.

TPR motifs are also found in cell division cycle genes, such as cdc16, cdc23, and cdc27, all encoding polypeptide components of the anaphase-promoting complex, which regulates cell cycle progression in mitosis. Mutations in the TPR regions of these complex members cause mitotic arrest prior to anaphase.

Another class of proteins, the SKD1 family of proteins contains a sole TPR motif. SKD1 family members, including VPS4, participate in intracellular protein trafficking, e.g., from the trans-Golgi network to the vacuole. This family of proteins can further include an AAA domain (an ATPase motif).

TPRs are also featured in proteins that regulate transcription, neurogenesis, protein kinase inhibition, NADPH oxidase, and protein folding. Thus, the TPR is a versatile and important polypeptide motif for regulating cell behaviors and physiology.

Dehydrogenases and tetratricopeptide repeat-containing protein have both been implicated in human disease. Consequently, the isolation and characterization of additional dehydrogenases and tetratricopeptide repeat-containing proteins will provide novel reagents for the treatment or prevention of disease, as well as new targets for the development of drugs that can be used to treat or prevent disease.

### SUMMARY OF THE 21617 AND 55562 INVENTION

The present invention is based, in part, on the discovery of novel dehydrogenase or tetratricopeptide repeat family members, referred to herein as “21617” or “55562”, respectively. The nucleotide sequence of a cDNA encoding 21617 is shown in SEQ ID NO:63, and the amino acid sequence of a 21617 polypeptide is shown in SEQ ID NO:64. The nucleotide sequence of a cDNA encoding 55562 is shown in SEQ ID NO:66, and the amino acid sequence of a 55562 polypeptide is shown in SEQ ID NO:67. In addition, the nucleotide sequences of the 21617 and 55562 coding regions are depicted in SEQ ID NO:65 and SEQ ID NO:68, respectively.

Accordingly, in one aspect, the invention features a nucleic acid molecule that encodes a 21617 or 55562 protein or polypeptide, e.g., a biologically active portion of the 21617 or 55562 protein. In a preferred embodiment the isolated nucleic acid molecule encodes a polypeptide having the amino acid sequence of SEQ ID NO:64 or SEQ ID NO:67. In other embodiments, the invention provides isolated 21617 or 55562 nucleic acid molecules having the nucleotide sequence shown in SEQ ID NO:63, SEQ ID NO:65, SEQ ID NO:66, or SEQ ID NO:68, or the sequence of a DNA insert of a plasmid deposited with ATCC Accession Number as described herein. In still other embodiments, the invention provides nucleic acid molecules that are substantially identical (e.g., naturally occurring allelic variants) to the nucleotide sequence shown in SEQ ID NO:63, SEQ ID NO:65, SEQ ID NO:66, or SEQ ID NO:68, or the sequence of a DNA insert of a plasmid deposited with ATCC Accession Number as described herein. In other embodiments, the invention provides a nucleic acid molecule which hybridizes under a stringency condition described herein to a nucleic acid molecule comprising the nucleotide sequence of SEQ ID NO:63, SEQ ID NO:65, SEQ ID NO:66, or SEQ ID NO:68, or the sequence of a DNA insert of a plasmid deposited with ATCC Accession Number as described herein, wherein the nucleic acid encodes a full length 21617 or 55562 protein or an active fragment thereof.

In a related aspect, the invention further provides nucleic acid constructs that include a 21617 or 55562 nucleic acid molecule described herein. In certain embodiments, the nucleic acid molecules of the invention are operatively linked to native or heterologous regulatory sequences. Also included, are vectors and host cells containing the 21617 or 55562 nucleic acid molecules of the invention e.g., vectors and host cells suitable for producing 21617 or 55562 nucleic acid molecules and polypeptides.

In another related aspect, the invention provides nucleic acid fragments suitable as primers or hybridization probes for the detection of 21617 or 55562-encoding nucleic acids.

In still another related aspect, isolated nucleic acid molecules that are antisense to a 21617 or 55562 encoding nucleic acid molecule are provided.

In another aspect, the invention features, 21617 or 55562 polypeptides, and biologically active or antigenic fragments thereof that are useful, e.g., as reagents or targets in assays applicable to treatment and diagnosis of 21617 or 55562-mediated or -related disorders. In another embodiment, the invention provides 21617 or 55562 polypeptides having a 21617 or 55562 activity. Preferred polypeptides are 21617 or 55562 proteins including at least one short chain dehydrogenase or tetratricopeptide repeat domain, and, preferably, having a 21617 or 55562 activity, e.g., a 21617 or 55562 activity as described herein.

In other embodiments, the invention provides 21617 or 55562 polypeptides, e.g., a 21617 or 55562 polypeptide having the amino acid sequence shown in SEQ ID NO:64 or SEQ ID NO:67, or the amino acid sequence encoded by the cDNA insert of a plasmid deposited with ATCC Accession Number as described herein; an amino acid sequence that is substantially identical to the amino acid sequence shown in SEQ ID NO:64 or SEQ ID NO:67, or the amino acid sequence encoded by the cDNA insert of a plasmid deposited with ATCC Accession Number as described herein; or an amino acid sequence encoded by a nucleic acid molecule having a nucleotide sequence which hybridizes under a stringency condition described herein to a nucleic acid molecule comprising the nucleotide sequence of SEQ ID NO:63, SEQ ID NO:65, SEQ ID NO:66, or SEQ ID NO:68, or the sequence of a DNA insert of a plasmid deposited with ATCC Accession Number as described herein, wherein the nucleic acid encodes a full length 21617 or 55562 protein or an active fragment thereof.

In a related aspect, the invention provides 21617 or 55562 polypeptides or fragments operatively linked to non-21617 or 55562 polypeptides to form fusion proteins.

In another aspect, the invention features antibodies and antigen-binding fragments thereof, that react with or, more preferably, specifically bind 21617 or 55562 polypeptides.

In another aspect, the invention provides methods of screening for agents, e.g., compounds, that modulate the expression or activity of a 21617 or 55562 polypeptide or nucleic acid.

In still another aspect, the invention provides a process for modulating 21617 or 55562 polypeptide or nucleic acid expression or activity, e.g. using the screened compounds. In certain embodiments, the methods involve treatment of conditions related to aberrant activity or expression of the 21617 or 55562 polypeptides or nucleic acids, such as conditions involving aberrant or deficient cellular proliferation and/or differentiation, metabolic disorders, neural disorders, or viral disorders.

In yet another aspect, the invention provides methods for modulating the activity of a 21617 or a 55562-expressing cell. In one embodiment, the methods inhibit the proliferation or induce the killing of the cell. The methods include contacting the cell with an agent, e.g., a compound (e.g., a compound identified using the methods described herein) that modulates the activity, or expression, of the 14094 polypeptide or nucleic acid, thereby modulating the activity of the 21617 or 55562-expressing cell.

In a preferred embodiment, the contacting step is effective in vitro or ex vivo. In other embodiments, the contacting step is effected in vivo, e.g., in a subject (e.g., a mammal, e.g., a human), as part of a therapeutic or prophylactic protocol.

In a preferred embodiment, the 21617 or 55562-expressing cell is a hyperproliferative cell is found in a solid tumor, a soft tissue tumor, or a metastatic lesion. Preferably, the tumor is a sarcoma, a carcinoma, or an adenocarcinoma. Preferably, the hyperproliferative cell is found in a cancerous or pre-cancerous tissue, e.g., a cancerous or pre-cancerous tissue where a 21617 or 55562 polypeptide or nucleic acid is expressed, e.g., breast, ovarian, colon, liver, lung, kidney, or brain cancer. Most preferably, the hyperproliferative cell is found in a tumor from the breast, ovary, colon, liver and lung.

In a preferred embodiment, the agent, e.g., the compound, is an inhibitor of a 21617 or 55562 polypeptide. Preferably, the inhibitor is chosen from a peptide, a phosphopeptide, a small organic molecule, a small inorganic molecule and an antibody (e.g., an antibody conjugated to a therapeutic moiety selected from a cytotoxin, a cytotoxic agent and a radioactive metal ion).

In a preferred embodiment, the agent, e.g., the compound, is an inhibitor of a 21617 or 55562 nucleic acid, e.g., an antisense, a ribozyme, or a triple helix molecule.

In some embodiments, the agent, e.g., compound is administered (e.g., to cells or to a subject) in combination with a second agent, e.g., a compound, e.g., a known therapeutic agent or compound. Such an agent, e.g., compound, could be used to treat or prevent cellular proliferation and differentiation disorders, metabolic disorders, neural disorders, or viral disorders. Examples of cytotoxic agents include anti-microtubule agent, a topoisomerase I inhibitor, a topoisomerase II inhibitor, an anti-metabolite, a mitotic inhibitor, an alkylating agent, an intercalating agent, an agent capable of interfering with a signal transduction pathway, an agent that promotes apoptosis or necrosis, and radiation.

In another aspect, the invention features methods for treating or preventing a disorder characterized by aberrant activity of a 21617 or 55562-expressing cell, in a subject. Preferably, the method includes administering to the subject (e.g., a mammal, e.g., a human) an effective amount of a compound (e.g., a compound identified using the methods described herein) that modulates the activity, or expression, of the 21617 or 55562 polypeptide or nucleic acid.

In a preferred embodiment, the disorder is a cancerous or pre-cancerous condition.

In a further aspect, the invention provides methods for evaluating the efficacy of a treatment of a disorder, e.g., a cellular proliferative and differentiative disorder, metabolic disorder, neural disorder, or viral disorder. The method includes: treating a subject, e.g., a patient or an animal, with a protocol under evaluation (e.g., treating a subject with one or more of: chemotherapy, radiation, and/or a compound identified using the methods described herein); and evaluating the expression of a 21617 or 55562 nucleic acid or polypeptide before and after treatment. A change, e.g., a decrease or increase, in the level of a 21617 or 55562 nucleic acid (e.g., mRNA) or polypeptide after treatment, relative to the level of expression before treatment, is indicative of the efficacy of the treatment of the disorder. The level of 21617 or 55562 nucleic acid or polypeptide expression can be detected by any method described herein.

In a preferred embodiment, the evaluating step includes obtaining a sample (e.g., a tissue sample, e.g., a biopsy, or a fluid sample) from the subject, before and after treatment and comparing the level of expressing of a 21617 or 55562 nucleic acid (e.g., mRNA) or polypeptide before and after treatment.

In another aspect, the invention provides methods for evaluating the efficacy of a therapeutic or prophylactic agent (e.g., an anti-neoplastic agent). The method includes: contacting a sample with an agent (e.g., a compound identified using the methods described herein or a cytotoxic agent) and, evaluating the expression of a 21617 or 55562 nucleic acid or polypeptide in the sample before and after the contacting step. A change, e.g., a decrease or increase, in the level of 21617 or 55562 nucleic acid (e.g., mRNA) or polypeptide in the sample obtained after the contacting step, relative to the level of expression in the sample before the contacting step, is indicative of the efficacy of the agent. The level of 21617 or 55562 nucleic acid or polypeptide expression can be detected by any method described herein. In a preferred embodiment, the sample includes cells obtained from the lung, colon, prostate, liver, breast, ovary, or cervix, or a cancerous tissue, e.g., a cancerous lung, colon, prostate, liver, breast, ovary, or cervix tissue.

The invention also provides assays for determining the activity of, or the presence or absence of, 21617 or 55562 polypeptides or nucleic acid molecules in a biological sample, including for disease diagnosis.

In further aspect, the invention provides assays for determining the presence or absence of a genetic alteration in a 21617 or 55562 polypeptide or nucleic acid molecule, including for disease diagnosis.

In another aspect, the invention features a two dimensional array having a plurality of addresses, each address of the plurality being positionally distinguishable from each other address of the plurality, and each address of the plurality having a unique capture probe, e.g., a nucleic acid or peptide sequence. At least one address of the plurality has a capture probe that recognizes a 21617 or 55562 molecule. In one embodiment, the capture probe is a nucleic acid, e.g., a probe complementary to a 21617 or 55562 nucleic acid sequence. In another embodiment, the capture probe is a polypeptide, e.g., an antibody specific for 21617 or 55562 polypeptides. Also featured is a method of analyzing a sample by contacting the sample to the aforementioned array and detecting binding of the sample to the array.

Other features and advantages of the invention will be apparent from the following detailed description, and from the claims.

### DETAILED DESCRIPTION OF 21617 AND 55562

Human 21617

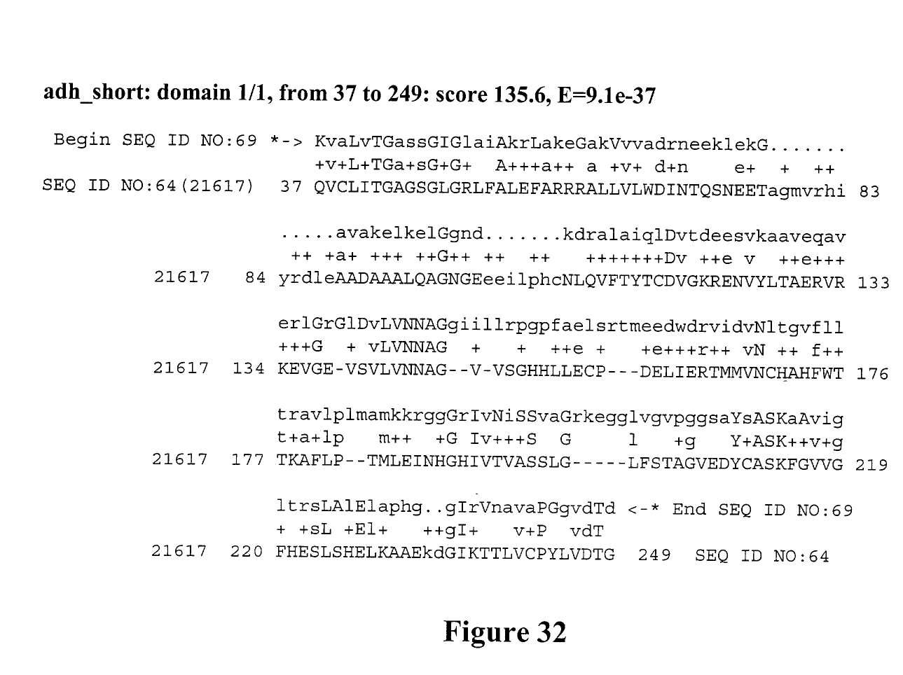

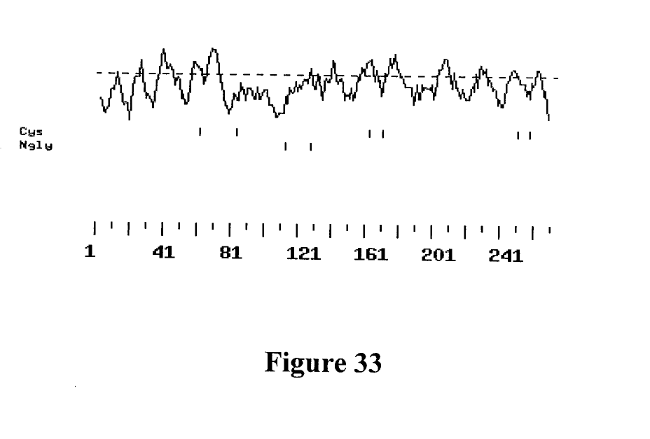

The human 21617 sequence (see SEQ ID NO:63, as recited in Example 6), which is approximately 3624 nucleotides long including untranslated regions, contains a predicted methionine-initiated coding sequence of about 1026 nucleotides, including the termination codon. The coding sequence encodes a 341 amino acid protein (see SEQ ID NO:64, as recited in Example 6). The human 21617 protein of SEQ ID NO:64 and FIG. 32 includes an amino-terminal hydrophobic amino acid sequence, consistent with a signal sequence, of about 21 amino acids (from amino acid 1 to about amino acid 21 of SEQ ID NO:64), which upon cleavage results in the production of a mature protein form). This mature protein form is approximately 319 amino acid residues in length (from about amino acid 22 to amino acid 341 of SEQ ID NO:64).

Human 21617 contains the following regions or other structural features:

a short chain dehydrogenase domain (PFAM Accession Number PF00106) located at about amino acid residues 37 to 249 of SEQ ID NO:64;

a predicted short-chain alcohol dehydrogenase family signature motif (PS00061) located at about amino acid residues 210 to 220 of SEQ ID NO:64;

a predicted signal peptide located at about amino acid residues 1 to 21 of SEQ ID NO:64, which when cleaved gives a predicted mature protein of 319 amino acids, from about amino acid residues 22 to 341 of SEQ ID NO:64;

two dileucine motifs located at about amino acid residues 62 to 63 and 154 to 155 of SEQ ID NO:64;

one predicted glycosaminoglycan attachment site (PS00002) located at about amino acid residues 46 to 49 of SEQ ID NO:64;

three predicted Protein Kinase C phosphorylation sites (PS00005) located at about amino acid residues 11 to 13, 176 to 178, and 289 to 291 of SEQ ID NO:64;

two predicted Casein Kinase II phosphorylation sites (PS00006) located at about amino acid residues 72 to 75, and 183 to 186 of SEQ ID NO:64;

six predicted N-myristoylation sites (PS00008) located at about amino acid residues 43 to 48, 147 to 152, 200 to 205, 235 to 240, 249 to 254, and 316 to 321 of SEQ ID NO:64; and

one predicted amidation site (PS00009) located at about amino acid residues 119 to 122 of SEQ ID NO:64.

For general information regarding PFAM identifiers, PS prefix and PF prefix domain identification numbers, refer to Sonnhammer et al. (1997) Protein 28:405-420 and http://www.psc.edu/general/software/packages/pfam/pfam.html.

A plasmid containing the nucleotide sequence encoding human 21617 (clone “Fbh21617FL”) was deposited with American Type Culture Collection (ATCC), 10801 University Boulevard, Manassas, Va. 20110-2209, on ______ and assigned Accession Number ______. This deposit will be maintained under the terms of the Budapest Treaty on the International Recognition of the Deposit of Microorganisms for the Purposes of Patent Procedure. This deposit was made merely as a convenience for those of skill in the art and is not an admission that a deposit is required under 35 U.S.C. §112.

Human 55562

The human 55562 sequence (see SEQ ID NO:66, as recited in Example 6), which is approximately 1327 nucleotides long including untranslated regions, contains a predicted methionine-initiated coding sequence of about 825 nucleotides, including the termination codon. The coding sequence encodes a 274 amino acid protein (see SEQ ID NO:67, as recited in Example 6).

Human 55562 contains the following regions or other structural features:

a tetratricopeptide repeat domain (PFAM Accession Number PF00515) located at about amino acid residues 40 to 73 of SEQ ID NO:67;

a PD314595 homology domain (ProDom Accession Number PD314595) located at about amino acid residues 40 to 266 of SEQ ID NO:67;

four predicted Protein Kinase C phosphorylation sites (PS00005) located at about amino acid residues 3 to 5, 22 to 24, 81 to 83, and 201 to 203 of SEQ ID NO:67;

four predicted Casein Kinase II phosphorylation sites (PS00006) located at about amino acid residues 139 to 142, 180 to 183, 216 to 219, 261 to 264 of SEQ ID NO:67;

three predicted cAMP/cGMP-dependent protein kinase phosphorylation sites (PS00004) located at about amino acid residues 5 to 8, 19 to 22, and 268 to 271 of SEQ ID NO:67;

two predicted N-glycosylation sites (PS00001) located at about amino acid residues 122 to 125, 137 to 140 of SEQ ID NO:67; and

one predicted N-myristylation sites (PS00008) located at about amino acid residues 76 to 81 of SEQ ID NO:67.

For general information regarding PFAM identifiers, PS prefix and PF prefix domain identification numbers, refer to Sonnhammer et al. (1997) Protein 28:405-420 and http://www.psc.edu/general/software/packages/pfam/pfam.html.

A plasmid containing the nucleotide sequence encoding human 55562 (clone “Fbh55562FL”) was deposited with American Type Culture Collection (ATCC), 10801 University Boulevard, Manassas, Va. 20110-2209, on ______ and assigned Accession Number ______. This deposit will be maintained under the terms of the Budapest Treaty on the International Recognition of the Deposit of Microorganisms for the Purposes of Patent Procedure. This deposit was made merely as a convenience for those of skill in the art and is not an admission that a deposit is required under 35 U.S.C. §112.

| | | | | ATCC |
|---|---|---|---|---|
| Gene Name | cDNA | Protein | Coding Region | accession number |
| 21617 | SEQ ID NO: 63 | SEQ ID NO: 64 | SEQ ID NO: 65 | |
| 55562 | SEQ ID NO: 66 | SEQ ID NO: 67 | SEQ ID NO: 68 | |

21617 Polypeptide Characteristics

The 21617 protein contains a significant number of structural characteristics in common with members of the short chain dehydrogenase family. The term “family” when referring to the protein and nucleic acid molecules of the invention means two or more proteins or nucleic acid molecules having a common structural domain or motif and having sufficient amino acid or nucleotide sequence homology as defined herein. Such family members can be naturally or non-naturally occurring and can be from either the same or different species. For example, a family can contain a first protein of human origin as well as other distinct proteins of human origin, or alternatively, can contain homologues of non-human origin, e.g., rat or mouse proteins. Members of a family can also have common functional characteristics.

A short chain dehydrogenase family of proteins is characterized by the presence of at least two domains; the first binds a coenzyme, such as NAD or NADP, and the second binds substrate. Sequence of the coenzyme domain does not appear to be conserved among dehydrogenases. The second domain determines substrate specificity and contains amino acids involved in catalysis.

Short-chain dehydrogenases/reductases (SDRs) typically function as dimers or tetramers. The subunits are composed of approximately 250 to 300 amino acid residues and include an N-terminal co-enzyme binding motif having the sequence G-X-X-X-G-X-G, and an active-site motif having the sequence Y-X-X-K (Opperman et al. (1999) Enzymology and Molecular Biology of Carbonyl Metabolism 7 ed. Weiner et al., Plenum Publishers, NY p. 373-377). Although identity between different SDR members is at the 15% to 30% level, three-dimensional structures thus far analyzed reveal a highly similar conformation consisting of a single subunit that includes seven to eight β-strands.

Members of short chain dehydrogenase family include alcohol dehydrogenase, 3-β-hydroxysteroid dehydrogenase, estradiol 17-β-dehydrogenase, retinal dehydrogenase, and NADPH-dependent carbonyl reductase. Thus, this family includes enzymes critical for the proper function of many physiological systems, including metabolism (e.g., alcohol metabolism, steroid metabolism, and the metabolism of toxins), and cellular proliferation and differentiation.

A 21617 polypeptide can include a “short chain dehydrogenase domain” or regions homologous with a “short chain dehydrogenase domain”.

As used herein, the terms “short chain dehydrogenase domain” or “dehydrogenase” includes an amino acid sequence of about 100 to 300 amino acid residues in length, having a bit score for the alignment of the sequence to the short chain dehydrogenase domain profile (PFAM HMM) of at least 70. Preferably, a short chain dehydrogenase domain includes at least about 140 to 280 amino acids, more preferably about 200 to 220 amino acid residues, and has a bit score for the alignment of the sequence to the short chain dehydrogenase domain (HMM) of at least 100, 125, 135, or greater. The short chain dehydrogenase domain (HMM) has been assigned the PFAM Accession Number PF00106. An alignment of the short chain dehydrogenase domain (amino acids 37 to 249 of SEQ ID NO:64) of human 21617 with a consensus amino acid sequence (SEQ ID NO:69) derived from a hidden Markov model is depicted in FIG. 32.

In a preferred embodiment 21617 polypeptide or protein has a “short chain dehydrogenase domain” or a region which includes at least about 100 to 300, more preferably about 140 to 280, or 200 to 220 amino acid residues and has at least about 50%, 60%, 70% 80% 90% 95%, 98%, 99%, or 100% homology with a “short chain dehydrogenase domain”, e.g., the short chain dehydrogenase domain of human 21617 (e.g., residues 37 to 249 of SEQ ID NO:64).

To identify the presence of a “short chain dehydrogenase” domain in a 21617 protein sequence, and make the determination that a polypeptide or protein of interest has a particular profile, the amino acid sequence of the protein can be searched against the PFAM database of HMMs (e.g., the PFAM database, release 2.1) using the default parameters. For example, the hmmsf program, which is available as part of the HMMER package of search programs, is a family specific default program for MILPAT0063 and a score of 15 is the default threshold score for determining a hit. Alternatively, the threshold score for determining a hit can be lowered (e.g., to 8 bits). A description of the PFAM database can be found in Sonhammer et al. (1997), Proteins 28(3):405-420, and a detailed description of HMMs can be found, for example, in Gribskov et al. (1990), Meth. Enzymol. 183:146-159; Gribskov et al. (1987), Proc. Natl. Acad. Sci. USA 84:4355-4358; Krogh et al. (1994), J. Mol. Biol. 235:1501-1531; and Stultz et al. (1993), Protein Sci. 2:305-314, the contents of which are incorporated herein by reference. A search was performed against the PFAM HMM database resulting in the identification of a “short chain dehydrogenase” domain in the amino acid sequence of human 21617 located at about amino acid residues 37 to 249 of SEQ ID NO:64 (see FIG. 32).

In some embodiments, a 21617 protein includes at least one dehydrogenase family signature motif. As used herein, a “dehydrogenase family signature motif” includes a sequence of at least eleven amino acid residues defined by the sequence: [LIVSPADNK]-X(12)-Y-[PSTAGNCV]-[STAGNQCIVM]-[STAGC]-K-{PC}-[SAGFYR]-[LIVMSTAGD]-X(2)-[LIVMFYW]-X(3)-[LIVMFYWGAPTHQ]-[GSACQRHM]. A dehydrogenase family signature motif, as defined, can be involved in the oxidation of a chemical group, e.g., an alcohol group (C—OH), or the reduction of a chemical group, e.g., a carbonyl group (C═O). A dehydrogenase family signature motif can include 16, 24, and even 29 amino acid residues. The dehydrogenase family signature motif has been given the PROSITE Accession Number PS00061.

In preferred embodiments, a 21617 polypeptide or protein has at least one dehydrogenase family signature motif, or a region which includes at least 11 amino acid residues and has at least 70%, 80%, 90%, or 100% homology with a “dehydrogenase family signature motif”, e.g., dehydrogenase family signature motif of human 21617, e.g., about amino acid residues 210 to 220 of SEQ ID NO:64.

In some embodiments, a 21617 molecule can further include a signal sequence. As used herein, a “signal peptide” or “signal sequence” refers to a peptide of about 15 to 50, preferably about 20 to 40, more preferably, 21 amino acid residues in length which occurs at the N-terminus of secretory and integral membrane proteins and which contains a majority of hydrophobic amino acid residues. For example, a signal sequence contains at least about 15 to 50, preferably about 20 to 40, more preferably, 21 amino acid residues, and has at least about 40-70%, preferably about 50-65%, and more preferably about 55-60% hydrophobic amino acid residues (e.g., alanine, valine, leucine, isoleucine, phenylalanine, tyrosine, tryptophan, or proline). Such a “signal sequence”, also referred to in the art as a “signal peptide”, serves to direct a protein containing such a sequence to a lipid bilayer. For example, in one embodiment, a 21617 protein contains a signal sequence located at about amino acid residues 1 to 21 of SEQ ID NO:64. The “signal sequence” is cleaved during processing of the mature protein. The mature 21617 protein corresponds to about amino acid residues 23 to 341 of SEQ ID NO:64.

In preferred embodiments, a 21617 polypeptide or protein has at least one predicted signal sequence, or a region which includes at least 15, 18, 20, or even 21 amino acid residues and has at least 70%, 80%, 90%, or 100% homology with a “signal sequence”, e.g., a signal sequence of human 21617, e.g., about amino acid residues 1 to 21 of SEQ ID NO:64.

A 21617 family member can include at least one short chain dehydrogenase domain. Furthermore, a 21617 family member can include at least one dehydrogenase family signature motif; at least one signal sequence; at least one, two, preferably three protein kinase C phosphorylation sites; at least one, preferably two casein kinase II phosphorylation sites; at least one, two, three, four, five, preferably six N-myristylation sites; and at least one amidation site.

As the 21617 polypeptides of the invention may modulate 21617-mediated activities, they may be useful as of for developing novel diagnostic and therapeutic agents for 21617-mediated or related disorders, as described below.

As used herein, a “21617 activity”, “biological activity of 21617” or “functional activity of 21617”, refers to an activity exerted by a 21617 protein, polypeptide or nucleic acid molecule. For example, a 21617 activity can be an activity exerted by 21617 in a physiological milieu on, e.g., a 21617-responsive cell or on a 21617 substrate, e.g., a small molecule (e.g. a steroid molecule or a toxin) or a protein. A 21617 activity can be determined in vivo or in vitro. In one embodiment, a 21617 activity is a direct activity, such as an association with a 21617 target molecule. A “target molecule” or “binding partner” is a molecule with which a 21617 protein binds or interacts in nature. In an exemplary embodiment, 21617 is an enzyme that oxidizes an alcohol group or reduces a carbonyl group found in a substrate.

A 21617 activity can also be an indirect activity, e.g., a cellular signaling activity mediated by interaction of a 21617 substrate with a receptor. The features of the 21617 molecules of the present invention can provide similar biological activities as short chain dehydrogenase family members. For example, the 21617 proteins of the present invention can have one or more of the following activities: (1) steroid biosynthesis or metabolism (breakdown); (2) developmental changes associated with steroid biosynthesis or metabolism (e.g., sex trait development); (3) metabolism or removal of natural or xenobiotic substances (e.g., ethanol, toxins, etc.); or (4) cellular proliferation or differentiation.

Furthermore, the 21617 molecules of the invention can be expected to function in the tissues where they are expressed, e.g., colon, breast, lung, cervis, ovary, liver, kidney, endothelial cells, and tumor tissue derived thereof. Thus, the 21617 molecules can act as novel diagnostic targets and therapeutic agents for controlling metabolic disorders, e.g., involving the metabolism of small molecules (e.g., steroids or alcohols), proliferation and differentiation disorders, e.g., cancer (e.g., colon, colorectal, breast, lung, cervical, ovarian or liver cancer), kidney disorders, or endothelial cell disorders.

55562 Polypeptide Characteristics

The 55562 protein contains a significant number of structural characteristics in common with members of the tetratricopeptide repeat (TPR) family. The term “family” when referring to the protein and nucleic acid molecules of the invention means two or more proteins or nucleic acid molecules having a common structural domain or motif and having sufficient amino acid or nucleotide sequence homology as defined herein. Such family members can be naturally or non-naturally occurring and can be from either the same or different species. For example, a family can contain a first protein of human origin as well as other distinct proteins of human origin, or alternatively, can contain homologues of non-human origin, e.g., rat or mouse proteins. Members of a family can also have common functional characteristics.

A common fold characterizes the TPR domains of the TPR family of proteins. TPR repeats can be highly degenerate. However, a pattern of small and large residues is required for the repeat to adopt the TPR fold. Each repeat of a TPR domain folds into an antiparallel pair of α-helices. Adjacent repeats can pack against one another in a parallel format to produce a right-handed super-helical structure with a continuous amphipathic groove, e.g., a possible binding site of an α-helix of an interaction partner (Das et al., supra).

TPR domains can serve a variety of functions, including scaffolding protein-protein interactions for complex formation and regulation of protein function. Consequently, TPRs have been found in proteins that regulate a variety of different processes, including transcription, neurogenesis, signal transduction, metabolism, and protein folding and trafficking.

A 55562 polypeptide can include a “TPR domain” or regions homologous with a “TPR domain”.

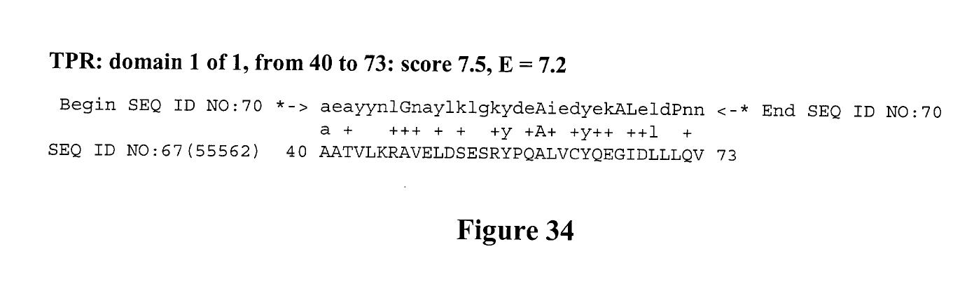

As used herein, the terms “tetratricopeptide repeat domain” or “TPR domain” include an amino acid sequence of about 20 to 45 amino acid residues in length and having a bit score for the alignment of the sequence to the TPR domain (HMM) of at least 5. Preferably, a TPR domain includes at least about 15 to 60 amino acids, more preferably about 20 to 45 amino acid residues, or about 27 to 36 amino acids and has a bit score for the alignment of the sequence to the TPR domain (HMM) of at least 1, 2, 3, 4, 5, 6, 7, or greater. Preferably, a TRP domain includes at least one small hydrophobic residue in both the first and second helix which are capable of interacting with one another such that interaction between the two helices is stabilized. In addition, a TRP domain can include a conserved aromatic residue. The TPR domain (HMM) has been assigned the PFAM Accession Number PF00515. An alignment of the TPR domain (amino acids 40 to 73 of SEQ ID NO:67) of human 55562 with a consensus amino acid sequence (SEQ ID NO:70) derived from a hidden Markov model is depicted in FIG. 34. As can be seen from the alignment, human 55562 includes alanine residues located at about amino acid residues 47 and 58 of SEQ ID NO:67, as well as a tyrosine residue located at about amino acid residue 55 of SEQ ID NO:67.

In a preferred embodiment, a 55562 polypeptide or protein has a “TPR domain” or a region which includes at least about 15 to 60 more preferably about 20 to 45 or 27 to 36, e.g., about 33 amino acid residues and has at least about 60%, 70%, 80%, 90%, 95%, 98%, 99%, or 100% homology with a “TPR domain,” e.g., the TPR domain of human 55562 (e.g., residues 40 to 73 of SEQ ID NO:67).

To identify the presence of a “TPR” domain in a 55562 protein sequence, and make the determination that a polypeptide or protein of interest has a particular profile, the amino acid sequence of the protein can be searched against the PFAM database of HMMs (e.g., the Pfam database, release 2.1) using the default parameters. For example, the hmmsf program, which is available as part of the HMMER package of search programs, is a family specific default program for MILPAT0063 and a score of 15 is the default threshold score for determining a hit. Alternatively, the threshold score for determining a hit can be lowered (e.g., to 8 bits). A description of the PFAM database can be found in Sonhammer et al. (1997) Proteins 28(3):405-420 and a detailed description of HMMs can be found, for example, in Gribskov et al. (1990) Meth. Enzymol. 183:146-159; Gribskov et al. (1987) Proc. Natl. Acad. Sci. USA 84:4355-4358; Krogh et al. (1994) J. Mol. Biol. 235:1501-1531; and Stultz et al. (1993) Protein Sci. 2:305-314, the contents of which are incorporated herein by reference. A search was performed against the HMM database resulting in the identification of a “TPR domain” domain in the amino acid sequence of human 55562 located at about amino acid residues 40 to 73 of SEQ ID NO:67 (see FIG. 34).

A 55562 family member can further include a “PD314595 homology domain” or regions homologous with a “PD314595 homology domain”.

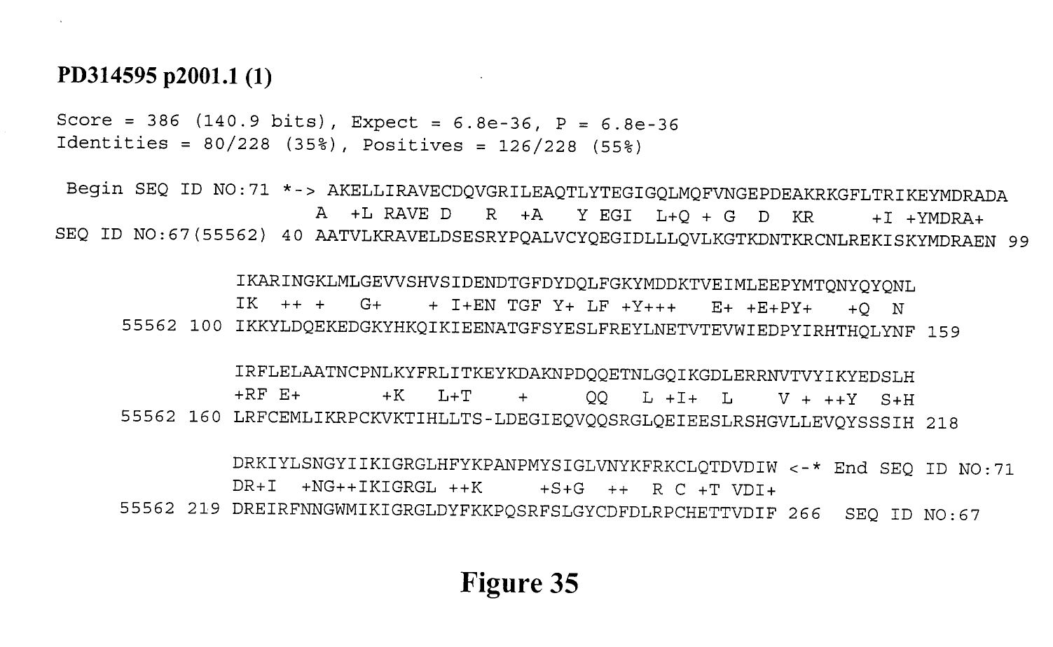

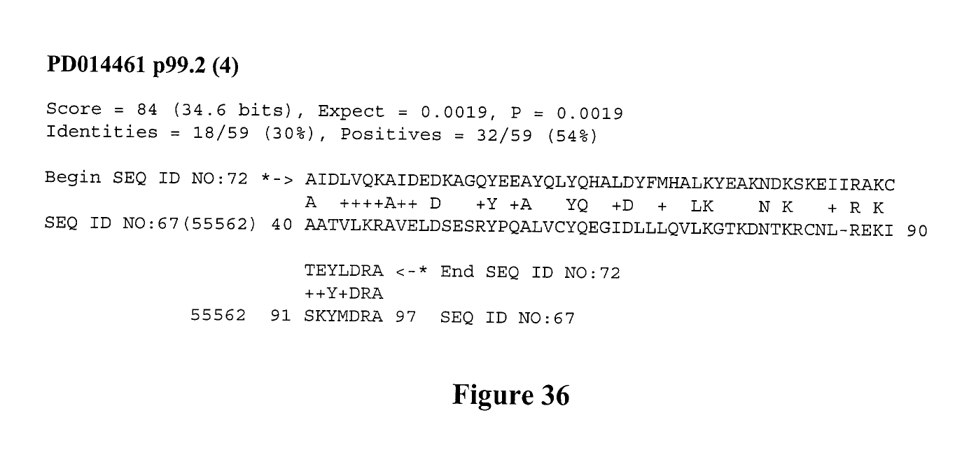

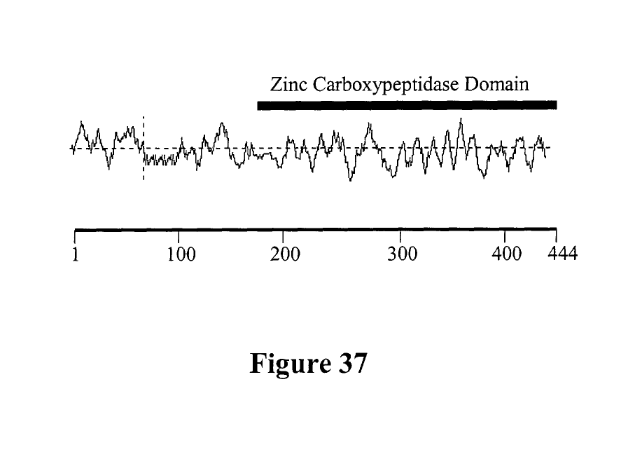

As used herein, the term “PD314595 homology domain” includes an amino acid sequence of about 150 to 300 amino acid residues in length and having a bit score for the alignment of the sequence to the TPR domain (HMM) of at least 70. Preferably, a PD314595 homology domain includes at least about 175 to 275 amino acids, more preferably about 200 to 250 amino acid residues, or about 220 to 235 amino acids and has a bit score for the alignment of the sequence to the TPR domain (HMM) of at least 100, 125, 130, 135, 140, or greater. Preferably, a PD314595 homology domain includes at least one tetratricopeptide repeat located near the N-terminus of the domain. The PD314595 homology domain has been given the ProDom accession number PD134595. An alignment of the PD314595 homology domain (about amino acids 40 to 266 of SEQ ID NO:67) of human 55562 with a consensus amino acid sequence (SEQ ID NO:71) is depicted in FIG. 35.

In a preferred embodiment, a 55562 polypeptide or protein has a “PD314595 homology domain” or a region which includes at least about 175 to 275, more preferably about 200 to 250, or about 220 to 235 amino acid residues and has at least about 60%, 70%, 80%, 90%, 95%, 98%, 99%, or 100% homology with a “PD314595 homology domain,” e.g., the PD314595 homology domain of human 55562 (e.g., residues 40 to 266 of SEQ ID NO:67).

To identify the presence of a “PD314595 homology domain” in a 55562 protein sequence, and make the determination that a polypeptide or protein of interest has a particular profile, the human 55562 amino acid sequence can be searched against the ProDom database of domains (Corpet et al. (1999), Nucl. Acids Res. 27:263-267). The ProDom protein domain database consists of an automatic compilation of homologous domains. Current versions of ProDom are built using recursive PSI-BLAST searches (Altschul SF et al. (1997) Nucleic Acids Res. 25:3389-3402; Gouzy et al. (1999) Computers and Chemistry 23:333-340.) of the SWISS-PROT 38 and TREMBL protein databases. The database automatically generates a consensus sequence for each domain. A BLAST search was performed against the ProDom database resulting in the identification of a consensus amino acid sequence for the PD314595 homology domain in the amino acid sequence of human 55562 at about residues 40 to 266 of SEQ ID NO:67 (see FIG. 35).

A 55562 family member can include at least one TPR domain and at least one PD314595 homology domain. Furthermore, a 55562 family member can include at least one, preferably two predicted N-glycosylation sites; at least one, two, three, preferably four protein kinase C phosphorylation sites (PS00005); at least one, two, three, preferably four predicted casein kinase II phosphorylation sites (PS00006); at least one, two, preferably three cAMP and cGMP-dependent protein kinase phosphorylation sites; and at least one predicted N-myristylation sites (PS00008).

As the 55562 polypeptides of the invention may modulate 55562-mediated activities, they may be useful as of for developing novel diagnostic and therapeutic agents for 55562-mediated or related disorders, as described below.

As used herein, a “55562 activity”, “biological activity of 55562” or “functional activity of 55562”, refers to an activity exerted by a 55562 protein, polypeptide or nucleic acid molecule on e.g., a 55562-responsive cell or on a 55562 substrate, e.g., a protein substrate, as determined in vivo or in vitro. In one embodiment, a 55562 activity is a direct activity, such as an association with a 55562 target molecule. A “target molecule” or “binding partner” is a molecule with which a 55562 protein binds or interacts in nature. In an exemplary embodiment, 55562 is a receptor, e.g., for a polyunsaturated fatty acid; a interface for binding a chaperone; or an interface for scaffolding with a protein complex. A 55562 activity can also be an indirect activity, e.g., a cellular signaling activity mediated by interaction of the 55562 protein with a 55562 receptor.

Based on the above-described sequence similarities, the 55562 molecules of the present invention are predicted to have similar biological activities as TPR family members. For example, the 55562 proteins of the present invention can have one or more of the following activities: (1) sensing a second messenger, e.g., a polyunsaturated fatty acid (e.g., arachidonic acid); (2) associating with other proteins so as to form a multimeric protein assembly; (3) allosterically inhibiting an enzyme activity, e.g., an anaphase promoting activity, a kinase activity, or a phosphatase activity; (4) regulating an intracellular trafficking pathway; (5) interfacing with intracellular trafficking landmark and regulator proteins; (6) regulation of metabolic processes including, e.g., regulation of metabolic enzymes, e.g., NADPH oxidase; or (7) inhibiting any of (1)-(6), e.g., via the formation of a dominant negative fragment of 55562.

Thus, the 55562 molecules can act as novel diagnostic targets and therapeutic agents for controlling cell proliferation and/or differentiation disorders, neural disorders (e.g., disorders of the brain), metabolic disorders, or viral disorders (e.g., as related to the viral inhibition of protein trafficking).

The 21617 or 55562 molecules can act as novel diagnostic targets and therapeutic agents for controlling one or more of cellular proliferative and/or differentiative disorders, metabolic disorders, kidney disorders, endothelial cell disorders, neural disorders (e.g., brain disorders), and viral disorders.

Examples of cellular proliferative and/or differentiative disorders include cancer, e.g., carcinoma, sarcoma, metastatic disorders or hematopoietic neoplastic disorders, e.g., leukemias. A metastatic tumor can arise from a multitude of primary tumor types, including but not limited to those of prostate, colon, lung, breast and liver origin.

As used herein, the terms “cancer”, “hyperproliferative” and “neoplastic” refer to cells having the capacity for autonomous growth. Examples of such cells include cells having an abnormal state or condition characterized by rapidly proliferating cell growth. Hyperproliferative and neoplastic disease states may be categorized as pathologic, i.e., characterizing or constituting a disease state, or may be categorized as non-pathologic, i.e., a deviation from normal but not associated with a disease state. The term is meant to include all types of cancerous growths or oncogenic processes, metastatic tissues or malignantly transformed cells, tissues, or organs, irrespective of histopathologic type or stage of invasiveness. “Pathologic hyperproliferative” cells occur in disease states characterized by malignant tumor growth. Examples of non-pathologic hyperproliferative cells include proliferation of cells associated with wound repair.

The terms “cancer” or “neoplasms” include malignancies of the various organ systems, such as affecting lung, breast, thyroid, lymphoid, gastrointestinal, and genito-urinary tract, as well as adenocarcinomas which include malignancies such as most colon cancers, renal-cell carcinoma, prostate cancer and/or testicular tumors, non-small cell carcinoma of the lung, cancer of the small intestine and cancer of the esophagus.

The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. Exemplary carcinomas include those forming from tissue of the cervix, lung, prostate, breast, head and neck, colon and ovary. The term also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures.

The term “sarcoma” is art recognized and refers to malignant tumors of mesenchymal derivation.

Examples of cellular proliferative and/or differentiative disorders of the colon include, but are not limited to, non-neoplastic polyps, adenomas, familial syndromes, colorectal carcinogenesis, colorectal carcinoma, and carcinoid tumors.

Examples of cellular proliferative and/or differentiative disorders of the liver include, but are not limited to, nodular hyperplasias, adenomas, and malignant tumors, including primary carcinoma of the liver and metastatic tumors.

Examples of cellular proliferative and/or differentiative disorders of the breast include, but are not limited to, proliferative breast disease including, e.g., epithelial hyperplasia, sclerosing adenosis, and small duct papillomas; tumors, e.g., stromal tumors such as fibroadenoma, phyllodes tumor, and sarcomas, and epithelial tumors such as large duct papilloma; carcinoma of the breast including in situ (noninvasive) carcinoma that includes ductal carcinoma in situ (including Paget's disease) and lobular carcinoma in situ, and invasive (infiltrating) carcinoma including, but not limited to, invasive ductal carcinoma, invasive lobular carcinoma, medullary carcinoma, colloid (mucinous) carcinoma, tubular carcinoma, and invasive papillary carcinoma, and miscellaneous malignant neoplasms. Disorders in the male breast include, but are not limited to, gynecomastia and carcinoma.

Examples of cellular proliferative and/or differentiative disorders of the lung include, but are not limited to, bronchogenic carcinoma, including paraneoplastic syndromes, bronchioloalveolar carcinoma, neuroendocrine tumors, such as bronchial carcinoid, miscellaneous tumors, and metastatic tumors; pathologies of the pleura, including inflammatory pleural effusions, noninflammatory pleural effusions, pneumothorax, and pleural tumors, including solitary fibrous tumors (pleural fibroma) and malignant mesothelioma.

Additional examples of proliferative disorders include hematopoietic neoplastic disorders. As used herein, the term “hematopoietic neoplastic disorders” includes diseases involving hyperplastic/neoplastic cells of hematopoietic origin. A hematopoietic neoplastic disorder can arise from myeloid, lymphoid or erythroid lineages, or precursor cells thereof. Preferably, the diseases arise from poorly differentiated acute leukemias, e.g., erythroblastic leukemia and acute megakaryoblastic leukemia. Additional exemplary myeloid disorders include, but are not limited to, acute promyeloid leukemia (APML), acute myelogenous leukemia (AML) and chronic myelogenous leukemia (CML) (reviewed in Vaickus, L. (1991) Crit Rev. in Oncol./Hemotol. 11:267-97); lymphoid malignancies include, but are not limited to acute lymphoblastic leukemia (ALL) which includes B-lineage ALL and T-lineage ALL, chronic lymphocytic leukemia (CLL), prolymphocytic leukemia (PLL), hairy cell leukemia (HLL) and Waldenstrom's macroglobulinemia (WM). Additional forms of malignant lymphomas include, but are not limited to non-Hodgkin lymphoma and variants thereof, peripheral T cell lymphomas, adult T cell leukemia/lymphoma (ATL), cutaneous T-cell Iymphoma (CTCL), large granular lymphocytic leukemia (LGF), Hodgkin's disease and Reed-Stemberg disease.

Examples of metabolic disorders include, but are not limited to, diseases of metabolic imbalance, e.g., obesity, anorexia nervosa, cachexia, lipid disorders (e.g., involving lipid production or storage, or the conversion of steroids, e.g., to active or inactive forms), and diabetes. In addition, metabolic disorders can be associated with an inability to eliminate toxins by enxymatically converting a toxin to an inactive form or, conversely, by converting a xenobiotic compound into a toxin.

Disorders involving the kidney include, but are not limited to, congenital anomalies including, but not limited to, cystic diseases of the kidney, that include but are not limited to, cystic renal dysplasia, autosomal dominant (adult) polycystic kidney disease, autosomal recessive (childhood) polycystic kidney disease, and cystic diseases of renal medulla, which include, but are not limited to, medullary sponge kidney, and nephronophthisis-uremic medullary cystic disease complex, acquired (dialysis-associated) cystic disease, such as simple cysts; glomerular diseases including pathologies of glomerular injury that include, but are not limited to, in situ immune complex deposition, that includes, but is not limited to, anti-GBM nephritis, Heymann nephritis, and antibodies against planted antigens, circulating immune complex nephritis, antibodies to glomerular cells, cell-mediated immunity in glomerulonephritis, activation of alternative complement pathway, epithelial cell injury, and pathologies involving mediators of glomerular injury including cellular and soluble mediators, acute glomerulonephritis, such as acute proliferative (poststreptococcal, postinfectious) glomerulonephritis, including but not limited to, poststreptococcal glomerulonephritis and nonstreptococcal acute glomerulonephritis, rapidly progressive (crescentic) glomerulonephritis, nephrotic syndrome, membranous glomerulonephritis (membranous nephropathy), minimal change disease (lipoid nephrosis), focal segmental glomerulosclerosis, membranoproliferative glomerulonephritis, IgA nephropathy (Berger disease), focal proliferative and necrotizing glomerulonephritis (focal glomerulonephritis), hereditary nephritis, including but not limited to, Alport syndrome and thin membrane disease (benign familial hematuria), chronic glomerulonephritis, glomerular lesions associated with systemic disease, including but not limited to, systemic lupus erythematosus, Henoch-Schonlein purpura, bacterial endocarditis, diabetic glomerulosclerosis, amyloidosis, fibrillary and immunotactoid glomerulonephritis, and other systemic disorders; diseases affecting tubules and interstitium, including acute tubular necrosis and tubulointerstitial nephritis, including but not limited to, pyelonephritis and urinary tract infection, acute pyelonephritis, chronic pyelonephritis and reflux nephropathy, and tubulointerstitial nephritis induced by drugs and toxins, including but not limited to, acute drug-induced interstitial nephritis, analgesic abuse nephropathy, nephropathy associated with nonsteroidal anti-inflammatory drugs, and other tubulointerstitial diseases including, but not limited to, urate nephropathy, hypercalcemia and nephrocalcinosis, and multiple myeloma; diseases of blood vessels including benign nephrosclerosis, malignant hypertension and accelerated nephrosclerosis, renal artery stenosis, and thrombotic microangiopathies including, but not limited to, classic (childhood) hemolytic-uremic syndrome, adult hemolytic-uremic syndrome/thrombotic thrombocytopenic purpura, idiopathic HUS/TTP, and other vascular disorders including, but not limited to, atherosclerotic ischemic renal disease, atheroembolic renal disease, sickle cell disease nephropathy, diffuse cortical necrosis, and renal infarcts; urinary tract obstruction (obstructive uropathy); urolithiasis (renal calculi, stones); and tumors of the kidney including, but not limited to, benign tumors, such as renal papillary adenoma, renal fibroma or hamartoma (renomedullary interstitial cell tumor), angiomyolipoma, and oncocytoma, and malignant tumors, including renal cell carcinoma (hypemephroma, adenocarcinoma of kidney), which includes urothelial carcinomas of renal pelvis.

Disorders involving endothelial cells include, but are not limited to, disorders characterized by aberrant, unregulated, or unwanted endothelial cell activity, e.g., proliferation, migration, angiogenesis, or vascularization; or aberrant expression of cell surface adhesion molecules or genes associated with angiogenesis, e.g., TIE-2, FLT and FLK. Endothelial cell disorders include tumorigenesis, tumor metastasis, psoriasis, diabetic retinopathy, endometriosis, Grave's disease, ischemic disease (e.g., atherosclerosis), and chronic inflammatory diseases (e.g., rheumatoid arthritis).

Disorders involving the brain include, but are not limited to, disorders involving neurons, and disorders involving glia, such as astrocytes, oligodendrocytes, ependymal cells, and microglia; cerebral edema, raised intracranial pressure and herniation, and hydrocephalus; malformations and developmental diseases, such as neural tube defects, forebrain anomalies, posterior fossa anomalies, and syringomyelia and hydromyelia; perinatal brain injury; cerebrovascular diseases, such as those related to hypoxia, ischemia, and infarction, including hypotension, hypoperfusion, and low-flow states—global cerebral ischemia and focal cerebral ischemia—infarction from obstruction of local blood supply, intracranial hemorrhage, including intracerebral (intraparenchymal) hemorrhage, subarachnoid hemorrhage and ruptured berry aneurysms, and vascular malformations, hypertensive cerebrovascular disease, including lacunar infarcts, slit hemorrhages, and hypertensive encephalopathy; infections, such as acute meningitis, including acute pyogenic (bacterial) meningitis and acute aseptic (viral) meningitis, acute focal suppurative infections, including brain abscess, subdural empyema, and extradural abscess, chronic bacterial meningoencephalitis, including tuberculosis and mycobacterioses, neurosyphilis, and neuroborreliosis (Lyme disease), viral meningoencephalitis, including arthropod-borne (Arbo) viral encephalitis, Herpes simplex virus Type 1, Herpes simplex virus Type 2, Varicalla-zoster virus (Herpes zoster), cytomegalovirus, poliomyelitis, rabies, and human immunodeficiency virus 1, including HIV-1 meningoencephalitis (subacute encephalitis), vacuolar myelopathy, AIDS-associated myopathy, peripheral neuropathy, and AIDS in children, progressive multifocal leukoencephalopathy, subacute sclerosing panencephalitis, fungal meningoencephalitis, other infectious diseases of the nervous system; transmissible spongiform encephalopathies (prion diseases); demyelinating diseases, including multiple sclerosis, multiple sclerosis variants, acute disseminated encephalomyelitis and acute necrotizing hemorrhagic encephalomyelitis, and other diseases with demyelination; degenerative diseases, such as degenerative diseases affecting the cerebral cortex, including Alzheimer disease and Pick disease, degenerative diseases of basal ganglia and brain stem, including Parkinsonism, idiopathic Parkinson disease (paralysis agitans), progressive supranuclear palsy, corticobasal degenration, multiple system atrophy, including striatonigral degenration, Shy-Drager syndrome, and olivopontocerebellar atrophy, and Huntington disease; spinocerebellar degenerations, including spinocerebellar ataxias, including Friedreich ataxia, and ataxia-telanglectasia, degenerative diseases affecting motor neurons, including amyotrophic lateral sclerosis (motor neuron disease), bulbospinal atrophy (Kennedy syndrome), and spinal muscular atrophy; inborn errors of metabolism, such as leukodystrophies, including Krabbe disease, metachromatic leukodystrophy, adrenoleukodystrophy, Pelizaeus-Merzbacher disease, and Canavan disease, mitochondrial encephalomyopathies, including Leigh disease and other mitochondrial encephalomyopathies; toxic and acquired metabolic diseases, including vitamin deficiencies such as thiamine (vitamin B₁) deficiency and vitamin B₁₂ deficiency, neurologic sequelae of metabolic disturbances, including hypoglycemia, hyperglycemia, and hepatic encephatopathy, toxic disorders, including carbon monoxide, methanol, ethanol, and radiation, including combined methotrexate and radiation-induced injury; tumors, such as gliomas, including astrocytoma, including fibrillary (diffuse) astrocytoma and glioblastoma multiforme, pilocytic astrocytoma, pleomorphic xanthoastrocytoma, and brain stem glioma, oligodendroglioma, and ependymoma and related paraventricular mass lesions, neuronal tumors, poorly differentiated neoplasms, including medulloblastoma, other parenchymal tumors, including primary brain lymphoma, germ cell tumors, and pineal parenchymal tumors, meningiomas, metastatic tumors, paraneoplastic syndromes, peripheral nerve sheath tumors, including schwannoma, neurofibroma, and malignant peripheral nerve sheath tumor (malignant schwannoma), and neurocutaneous syndromes (phakomatoses), including neurofibromotosis, including Type 1 neurofibromatosis (NF1) and TYPE 2 neurofibromatosis (NF2), tuberous sclerosis, and Von Hippel-Lindau disease.

Examples of viral disorders include, but are not limited to, Hepatitis B, Hepatitis C, Herpes Simplex Virus (HSV), and HIV. Viral diseases are typically associated with cell death and tissue fibrosis, especially liver fibrosis, epithelial sores, or loss of white blood cells. In addition, viruses can cause virus-associated carcinoma, especially hepatocellular cancer.

The 21617 or 55562 protein, fragments thereof, and derivatives and other variants of the sequences in SEQ ID NO:64 or SEQ ID NO:67 thereof are collectively referred to as “polypeptides or proteins of the invention” or “21617 polypeptides or proteins” or “55562 polypeptides or proteins”. Nucleic acid molecules encoding such polypeptides or proteins are collectively referred to as “nucleic acids of the invention” or “21617 nucleic acids” or “55562 nucleic acids.” 21617 or 55562 molecules refer to 21617 or 55562 nucleic acids, polypeptides, and antibodies.

As used herein, the term “nucleic acid molecule” includes DNA molecules (e.g., a cDNA or genomic DNA), RNA molecules (e.g., an mRNA) and analogs of the DNA or RNA. A DNA or RNA analog can be synthesized from nucleotide analogs. The nucleic acid molecule can be single-stranded or double-stranded, but preferably is double-stranded DNA.

The term “isolated nucleic acid molecule” or “purified nucleic acid molecule” includes nucleic acid molecules that are separated from other nucleic acid molecules present in the natural source of the nucleic acid. For example, with regards to genomic DNA, the term “isolated” includes nucleic acid molecules which are separated from the chromosome with which the genomic DNA is naturally associated. Preferably, an “isolated” nucleic acid is free of sequences which naturally flank the nucleic acid (i.e., sequences located at the 5′ and/or 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb or 0.1 kb of 5′ and/or 3′ nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material, or culture medium when produced by recombinant techniques, or substantially free of chemical precursors or other chemicals when chemically synthesized.

As used herein, the term “hybridizes under low stringency, medium stringency, high stringency, or very high stringency conditions” describes conditions for hybridization and washing. Guidance for performing hybridization reactions can be found in Current Protocols in Molecular Biology, John Wiley & Sons, N.Y. (1989), 6.3.1-6.3.6, which is incorporated by reference. Aqueous and nonaqueous methods are described in that reference and either can be used. Specific hybridization conditions referred to herein are as follows: 1) low stringency hybridization conditions in 6× sodium chloride/sodium citrate (SSC) at about 45° C., followed by two washes in 0.2× SSC, 0.1% SDS at least at 50° C. (the temperature of the washes can be increased to 55° C. for low stringency conditions); 2) medium stringency hybridization conditions in 6× SSC at about 45° C., followed by one or more washes in 0.2× SSC, 0.1% SDS at 60° C.; 3) high stringency hybridization conditions in 6× SSC at about 45° C., followed by one or more washes in 0.2× SSC, 0.1% SDS at 65° C.; and preferably 4) very high stringency hybridization conditions are 0.5M sodium phosphate, 7% SDS at 65° C., followed by one or more washes at 0.2× SSC, 1% SDS at 65° C. Very high stringency conditions (4) are the preferred conditions and the ones that should be used unless otherwise specified.

Preferably, an isolated nucleic acid molecule of the invention that hybridizes under a stringency condition described herein to the sequence of SEQ ID NO:63, SEQ ID NO:65, SEQ ID NO:66, or SEQ ID NO:68, corresponds to a naturally-occurring nucleic acid molecule.

As used herein, a “naturally-occurring” nucleic acid molecule refers to an RNA or DNA molecule having a nucleotide sequence that occurs in nature. For example a naturally occurring nucleic acid molecule can encode a natural protein.

As used herein, the terms “gene” and “recombinant gene” refer to nucleic acid molecules that include at least an open reading frame encoding a 21617 or 55562 protein. The gene can optionally further include non-coding sequences, e.g., regulatory sequences and introns. Preferably, a gene encodes a mammalian 21617 or 55562 protein or derivative thereof.

An “isolated” or “purified” polypeptide or protein is substantially free of cellular material or other contaminating proteins from the cell or tissue source from which the protein is derived, or substantially free from chemical precursors or other chemicals when chemically synthesized. “Substantially free” means that a preparation of 21617 or 55562 protein is at least 10% pure. In a preferred embodiment, the preparation of 21617 or 55562 protein has less than about 30%, 20%, 10% and more preferably 5% (by dry weight), of non-21617 or 55562 protein (also referred to herein as a “contaminating protein”), or of chemical precursors or non-21617 or 55562 chemicals. When the 21617 or 55562 protein or biologically active portion thereof is recombinantly produced, it is also preferably substantially free of culture medium, i.e., culture medium represents less than about 20%, more preferably less than about 10%, and most preferably less than about 5% of the volume of the protein preparation. The invention includes isolated or purified preparations of at least 0.01, 0.1, 1.0, and 10 milligrams in dry weight.

A “non-essential” amino acid residue is a residue that can be altered from the wild-type sequence of 21617 or 55562 without abolishing or substantially altering a 21617 or 55562 activity. Preferably the alteration does not substantially alter the 21617 or 55562 activity, e.g., the activity is at least 20%, 40%, 60%, 70% or 80% of wild-type. An “essential” amino acid residue is a residue that, when altered from the wild-type sequence of 21617 or 55562, results in abolishing a 21617 or 55562 activity such that less than 20% of the wild-type activity is present. For example, conserved amino acid residues in 21617 or 55562 are predicted to be particularly unamenable to alteration.

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in a 21617 or 55562 protein is preferably replaced with another amino acid residue from the same side chain family. Alternatively, in another embodiment, mutations can be introduced randomly along all or part of a 21617 or 55562 coding sequence, such as by saturation mutagenesis, and the resultant mutants can be screened for 21617 or 55562 biological activity to identify mutants that retain activity. Following mutagenesis SEQ ID NO:63, SEQ ID NO:65, SEQ ID NO:66, or SEQ ID NO:68, the encoded protein can be expressed recombinantly and the activity of the protein can be determined.

As used herein, a “biologically active portion” of a 21617 or 55562 protein includes a fragment of a 21617 or 55562 protein which participates in an interaction, e.g., an intramolecular or an inter-molecular interaction. An inter-molecular interaction can be a specific binding interaction or an enzymatic interaction (e.g., the interaction can be transient and a covalent bond is formed or broken). An inter-molecular interaction can be between a 21617 or 55562 molecule and a non-21617 or 55562 molecule or between a first 21617 or 55562 molecule and a second 21617 or 55562 molecule (e.g., a dimerization interaction). Biologically active portions of a 21617 or 55562 protein include peptides comprising amino acid sequences sufficiently homologous to or derived from the amino acid sequence of the 21617 or 55562 protein, e.g., the amino acid sequence shown in SEQ ID NO:64 or SEQ ID NO:67, which include less amino acids than the full length 21617 or 55562 proteins, and exhibit at least one activity of a 21617 or 55562 protein. Typically, biologically active portions comprise a domain or motif with at least one activity of the 21617 or 55562 protein, e.g., hydrolase activity or the ability to contribute to the formation of complexes, e.g., by interacting with other tetritricopeptide proteins. A biologically active portion of a 21617 or 55562 protein can be a polypeptide which is, for example, 10, 25, 50, 100, 200 or more amino acids in length. Biologically active portions of a 21617 or 55562 protein can be used as targets for developing agents which modulate a 21617 or 55562 mediated activity, e.g., hydrolase activity or the ability to contribute to the formation of complexes, e.g., by interacting with other tetritricopeptide proteins.

Calculations of homology or sequence identity between sequences (the terms are used interchangeably herein) are performed as follows.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, 60%, and even more preferably at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”).

The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch ((1970) J. Mol. Biol. 48:444-453) algorithm which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. A particularly preferred set of parameters (and the one that should be used unless otherwise specified) are a Blossum 62 scoring matrix with a gap penalty of 12, a gap extend penalty of 4, and a frameshift gap penalty of 5.

The percent identity between two amino acid or nucleotide sequences can be determined using the algorithm of E. Meyers and W. Miller ((1989) CABIOS, 4:11-17) which has been incorporated into the ALIGN program (version 2.0), using a PAM120 weight residue table, a gap length penalty of 12 and a gap penalty of 4.

The nucleic acid and protein sequences described herein can be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to 21617 or 55562 nucleic acid molecules of the invention. BLAST protein searches can be performed with the XBLAST program, score=50, wordlength=3 to obtain amino acid sequences homologous to 21617 or 55562 protein molecules of the invention. To obtain gapped alignments for comparison purposes, Gapped BLAST can be utilized as described in Altschul et al., (1997) Nucleic Acids Res. 25:3389-3402. When utilizing BLAST and Gapped BLAST programs, the default parameters of the respective programs (e.g., XBLAST and NBLAST) can be used. See http://www.ncbi.nlm.nih.gov.

Particularly preferred 21617 or 55562 polypeptides of the present invention have an amino acid sequence substantially identical to the amino acid sequence of SEQ ID NO:64 or SEQ ID NO:67. In the context of an amino acid sequence, the term “substantially identical” is used herein to refer to a first amino acid that contains a sufficient or minimum number of amino acid residues that are i) identical to, or ii) conservative substitutions of aligned amino acid residues in a second amino acid sequence such that the first and second amino acid sequences can have a common structural domain and/or common functional activity. For example, amino acid sequences that contain a common structural domain having at least about 60%, or 65% identity, likely 75% identity, more likely 85%, 90%. 91%, 92%, 93%, 94%, 95%, 96%, 97%, 98% or 99% identity to SEQ ID NO:64 or SEQ ID NO:67 are termed substantially identical.

In the context of nucleotide sequence, the term “substantially identical” is used herein to refer to a first nucleic acid sequence that contains a sufficient or minimum number of nucleotides that are identical to aligned nucleotides in a second nucleic acid sequence such that the first and second nucleotide sequences encode a polypeptide having common functional activity, or encode a common structural polypeptide domain or a common functional polypeptide activity. For example, nucleotide sequences having at least about 60%, or 65% identity, likely 75% identity, more likely 85%, 90%. 91%, 92%, 93%, 94%, 95%, 96%, 97%, 98% or 99% identity to SEQ ID NO:63, SEQ ID NO:65, SEQ ID NO:66, or SEQ ID NO:68 are termed substantially identical.

“Misexpression or aberrant expression”, as used herein, refers to a non-wildtype pattern of gene expression at the RNA or protein level. It includes: expression at non-wild type levels, i.e., over- or under-expression; a pattern of expression that differs from wild type in terms of the time or stage at which the gene is expressed, e.g., increased or decreased expression (as compared with wild type) at a predetermined developmental period or stage; a pattern of expression that differs from wild type in terms of altered, e.g., increased or decreased, expression (as compared with wild type) in a predetermined cell type or tissue type; a pattern of expression that differs from wild type in terms of the splicing size, translated amino acid sequence, post-transitional modification, or biological activity of the expressed polypeptide; a pattern of expression that differs from wild type in terms of the effect of an environmental stimulus or extracellular stimulus on expression of the gene, e.g., a pattern of increased or decreased expression (as compared with wild type) in the presence of an increase or decrease in the strength of the stimulus.

“Subject,” as used herein, refers to human and non-human animals. The term “non-human animals” of the invention includes all vertebrates, e.g., mammals, such as non-human primates (particularly higher primates), sheep, dog, rodent (e.g., mouse or rat), guinea pig, goat, pig, cat, rabbits, cow, and non-mammals, such as chickens, amphibians, reptiles, etc. In a preferred embodiment, the subject is a human. In another embodiment, the subject is an experimental animal or animal suitable as a disease model.

A “purified preparation of cells”, as used herein, refers to an in vitro preparation of cells. In the case cells from multicellular organisms (e.g., plants and animals), a purified preparation of cells is a subset of cells obtained from the organism, not the entire intact organism. In the case of unicellular microorganisms (e.g., cultured cells and microbial cells), it consists of a preparation of at least 10% and more preferably 50% of the subject cells.

Various aspects of the invention are described in further detail below.

Isolated Nucleic Acid Molecules of 21617 and 55562

In one aspect, the invention provides, an isolated or purified, nucleic acid molecule that encodes a 21617 or 55562 polypeptide described herein, e.g., a full-length 21617 or 55562 protein or a fragment thereof, e.g., a biologically active portion of a 21617 or 55562 protein. Also included is a nucleic acid fragment suitable for use as a hybridization probe, which can be used, e.g., to identify a nucleic acid molecule encoding a polypeptide of the invention, 21617 or 55562 mRNA, and fragments suitable for use as primers, e.g., PCR primers for the amplification or mutation of nucleic acid molecules.

In one embodiment, an isolated nucleic acid molecule of the invention includes the nucleotide sequence shown in SEQ ID NO:63 or SEQ ID NO:66, or a portion of either of these nucleotide sequences. In one embodiment, the nucleic acid molecule includes sequences encoding the human 21617 or 55562 protein (i.e., “the coding region” of SEQ ID NO:63 or SEQ ID NO:66, as shown in SEQ ID NO:65 and SEQ ID NO:68, respectively), as well as 5′ untranslated sequences. Alternatively, the nucleic acid molecule can include only the coding region of SEQ ID NO:63 or SEQ ID NO:66 (e.g., SEQ ID NO:65 or SEQ ID NO:68, respectively) and, e.g., no flanking sequences which normally accompany the subject sequence. In another embodiment, the nucleic acid molecule encodes a sequence corresponding to a fragment of a 21617 protein from about amino acid residues 37 to 249 of SEQ ID NO:64, or a fragment of a 55562 protein from about amino acid residues 40 to 73 of SEQ ID NO:67.

In another embodiment, an isolated nucleic acid molecule of the invention includes a nucleic acid molecule which is a full complement of the nucleotide sequence shown in SEQ ID NO:63, SEQ ID NO:65, SEQ ID NO:66, or SEQ ID NO:68, or a portion of any of these nucleotide sequences. In other embodiments, the nucleic acid molecule of the invention is sufficiently complementary to the nucleotide sequence shown in SEQ ID NO:63, SEQ ID NO:65, SEQ ID NO:66, or SEQ ID NO:68, such that it can hybridize (e.g., under a stringency condition described herein) to the nucleotide sequence shown in SEQ ID NO:63, SEQ ID NO:65, SEQ ID NO:66, or SEQ ID NO:68, thereby forming a stable duplex.

In one embodiment, an isolated nucleic acid molecule of the present invention includes a nucleotide sequence which is at least about: 60%, 65%, 70%, 75%, 80%, 85%, 90%, 91%, 92%, 93%, 94%, 95%, 96%, 97%, 98%, 99%, or more homologous to the entire length of the nucleotide sequence shown in SEQ ID NO:63, SEQ ID NO:65, SEQ ID NO:66, or SEQ ID NO:68, or a portion, preferably of the same length, of any of these nucleotide sequences.

21617 Nucleic Acid Fragments

A nucleic acid molecule of the invention can include only a portion of the nucleic acid sequence of SEQ ID NO:63 or SEQ ID NO:65. For example, such a nucleic acid molecule can include a fragment which can be used as a probe or primer or a fragment encoding a portion of a 21617 protein, e.g., an immunogenic or biologically active portion of a 21617 protein. A fragment can comprise those nucleotides of SEQ ID NO:63, which encode a short chain dehydrogenase domain of human 21617. The nucleotide sequence determined from the cloning of the 21617 gene allows for the generation of probes and primers designed for use in identifying and/or cloning other 21617 family members, or fragments thereof, as well as 21617 homologues, or fragments thereof, from other species.

In another embodiment, a nucleic acid includes a nucleotide sequence that includes part, or all, of the coding region and extends into either (or both) the 5′ or 3′ noncoding region. Other embodiments include a fragment which includes a nucleotide sequence encoding an amino acid fragment described herein. Nucleic acid fragments can encode a specific domain or site described herein or fragments thereof, particularly fragments thereof which are at least 50 amino acids in length, preferably 70, 80, 90, 100, 125, 150, 200, 250, 300, or 325 amino acids in length. Fragments also include nucleic acid sequences corresponding to specific amino acid sequences described above or fragments thereof. Nucleic acid fragments should not to be construed as encompassing those fragments that may have been disclosed prior to the invention.

A nucleic acid fragment can include a sequence corresponding to a domain, region, or functional site described herein. A nucleic acid fragment can also include one or more domains, regions, or functional sites described herein. Thus, for example, a 21617 nucleic acid fragment can include a sequence corresponding to a short chain dehydrogenase domain, a dehydrogenase family signature motif, or a signal peptide domain.

21617 probes and primers are provided. Typically a probe/primer is an isolated or purified oligonucleotide. The oligonucleotide typically includes a region of nucleotide sequence that hybridizes under a stringency condition described herein to at least about 7, 12 or 15, preferably about 20 or 25, more preferably about 30, 35, 40, 45, 50, 55, 60, 65, or 75 consecutive nucleotides of a sense or antisense sequence of SEQ ID NO:63 or SEQ ID NO:65, or of a naturally occurring allelic variant or mutant of SEQ ID NO:63 or SEQ ID NO:65. Preferably, an oligonucleotide is less than about 200, 150, 120, or 100 nucleotides in length.

In one embodiment, the probe or primer is attached to a solid support, e.g., a solid support described herein.

One exemplary kit of primers includes a forward primer that anneals to the coding strand and a reverse primer that anneals to the non-coding strand. The forward primer can anneal to the start codon, e.g., the nucleic acid sequence encoding amino acid residue 1 of SEQ ID NO:64. The reverse primer can anneal to the ultimate codon, e.g., the codon immediately before the stop codon, e.g., the codon encoding amino acid residue 341 of SEQ ID NO:64. In a preferred embodiment, the annealing temperatures of the forward and reverse primers differ by no more than 5, 4, 3, or 2° C.

In a preferred embodiment the nucleic acid is a probe which is at least 10, 12, 15, 18, 20 and less than 200, more preferably less than 100, or less than 50, nucleotides in length. It should be identical, or differ by 1, or 2, or less than 5 or 10 nucleotides, from a sequence disclosed herein. If alignment is needed for this comparison the sequences should be aligned for maximum homology. “Looped” out sequences from deletions or insertions, or mismatches, are considered differences.

A probe or primer can be derived from the sense or anti-sense strand of a nucleic acid which encodes a short chain dehydrogenase domain, e.g., the domain at about amino acid 37 to 249 of SEQ ID NO:64.

In another embodiment a set of primers is provided, e.g., primers suitable for use in a PCR, which can be used to amplify a selected region of a 21617 sequence, e.g., a domain, region, site or other sequence described herein. The primers should be at least 5, 10, or 50 base pairs in length and less than 100, or less than 200, base pairs in length. The primers should be identical, or differs by one base from a sequence disclosed herein or from a naturally occurring variant. For example, primers suitable for amplifying all or a portion of any of the following regions are provided: a short chain dehydrogenase domain from about amino acid 37 to 249 of SEQ ID NO:64.

A nucleic acid fragment can encode an epitope bearing region of a polypeptide described herein.

A nucleic acid fragment encoding a “biologically active portion of a 21617 polypeptide” can be prepared by isolating a portion of the nucleotide sequence of SEQ ID NO:63 or SEQ ID NO:65, which encodes a polypeptide having a 21617 biological activity (e.g., the biological activities of the 21617 proteins are described herein), expressing the encoded portion of the 21617 protein (e.g., by recombinant expression in vitro) and assessing the activity of the encoded portion of the 21617 protein. For example, a nucleic acid fragment encoding a biologically active portion of 21617 includes a short chain dehydrogenase domain, e.g., amino acid residues about 37 to 249 of SEQ ID NO:64. A nucleic acid fragment encoding a biologically active portion of a 21617 polypeptide, may comprise a nucleotide sequence which is greater than 300 or more nucleotides in length.

In preferred embodiments, a nucleic acid includes a nucleotide sequence which is about 300, 400, 500, 600, 700, 800, 900, 1000, 1100, 1200, 1300, 1400, 1500, 1600, 1700, 1800, 1900, 2000, 2200, 2400, 2600, 2800, 3000, 3200, 3400, 3600, or more nucleotides in length and hybridizes under a stringency condition described herein to a nucleic acid molecule of SEQ ID NO:63, or SEQ ID NO:65.

In a preferred embodiment, a nucleic acid fragment differs by at least 1, 2, 3, 10, 20, or more nucleotides from the sequence of Genbank accession number AF286885, G28278, G29385, or G25914. In some preferred embodiments, a nucleic acid fragment differs by at least 1, 2, 3, 10, 20, or more nucleotides from a sequence disclosed in one or more of WO 00/09552, WO 01/51628, WO 01/55301, WO 01/55364, WO 01/77289, WO 01/5719, EP1033401 or WO 01/02568. Differences can include differing in length or sequence identity. For example, a nucleic acid fragment can: include one or more nucleotides from SEQ ID NO:63 or SEQ ID NO:65 located outside the region of nucleotides 475-3607; not include all of the nucleotides of Genbank accession number AF286885, G28278, G29385, or G25914 and a sequence disclosed in one or more of WO 00/09552, WO 01/51628, WO 01/55301, WO 01/55364, WO 01/77289, WO 01/5719, EP1033401 or WO 01/02568, e.g., can be one or more nucleotides shorter (at one or both ends) than a sequence disclosed in Genbank accession number AF286885, G28278, G29385, or G25914 or a sequence disclosed in one or more of WO 00/09552, WO 01/51628, WO 01/55301, WO 01/55364, WO 01/77289, WO 01/5719, EP1033401 or WO 01/02568; or can differ by one or more nucleotides in the region of overlap.

55562 Nucleic Acid Fragments

A nucleic acid molecule of the invention can include only a portion of the nucleic acid sequence of SEQ ID NO:66 or SEQ ID NO:68. For example, such a nucleic acid molecule can include a fragment that can be used as a probe or primer or a fragment encoding a portion of a 55562 protein, e.g., an immunogenic or biologically active portion of a 55562 protein. A fragment can comprise those nucleotides of SEQ ID NO:66 or SEQ ID NO:68, which encode a tetratricopeptide repeat domain of human 55562. The nucleotide sequence determined from the cloning of the 55562 gene allows for the generation of probes and primers designed for use in identifying and/or cloning other 55562 family members, or fragments thereof, as well as 55562 homologues, or fragments thereof, from other species.

In another embodiment, a nucleic acid includes a nucleotide sequence that includes part, or all, of the coding region and extends into either (or both) the 5′ or 3′ noncoding region. Other embodiments include a fragment that includes a nucleotide sequence encoding an amino acid fragment described herein. Nucleic acid fragments can encode a specific domain or site described herein or fragments thereof, particularly fragments thereof which are at least 50, 79, 91, 100, 111, or more amino acids in length. Fragments also include nucleic acid sequences corresponding to specific amino acid sequences described above or fragments thereof. Nucleic acid fragments should not to be construed as encompassing those fragments that may have been disclosed prior to the invention.

A nucleic acid fragment can include a sequence corresponding to a domain, region, or functional site described herein. A nucleic acid fragment can also include one or more domains, regions, or functional sites described herein. Thus, for example, a 55562 nucleic acid fragment can include a sequence corresponding to a tetratricopeptide repeat domain, or a PD314595 homology domain.

55562 probes and primers are provided. Typically a probe/primer is an isolated or purified oligonucleotide. The oligonucleotide typically includes a region of nucleotide sequence that hybridizes under a stringency condition described herein to at least about 7, 12 or 15, preferably about 20 or 25, more preferably about 30, 35, 40, 45, 50, 55, 60, 65, or 75 consecutive nucleotides of a sense or antisense sequence of SEQ ID NO:66 or SEQ ID NO:68, or of a naturally occurring allelic variant or mutant of SEQ ID NO:66 or SEQ ID NO:68. Preferably, an oligonucleotide is less than about 200, 150, 120, or 100 nucleotides in length.

In one embodiment, the probe or primer is attached to a solid support, e.g., a solid support described herein.

One exemplary kit of primers includes a forward primer that anneals to the coding strand and a reverse primer that anneals to the non-coding strand. The forward primer can anneal to the start codon, e.g., the nucleic acid sequence encoding amino acid residue 1 of SEQ ID NO:67. The reverse primer can anneal to the ultimate codon, e.g., the codon immediately before the stop codon, e.g., the codon encoding amino acid residue 274 of SEQ ID NO:67. In a preferred embodiment, the annealing temperatures of the forward and reverse primers differ by no more than 5, 4, 3, or 2° C.

In a preferred embodiment the nucleic acid is a probe which is at least 10, 12, 15, 18, 20 and less than 200, more preferably less than 100, or less than 50, nucleotides in length. It should be identical, or differ by 1, or 2, or less than 5 or 10 nucleotides, from a sequence disclosed herein. If alignment is needed for this comparison the sequences should be aligned for maximum homology. “Looped” out sequences from deletions or insertions, or mismatches, are considered differences.

A probe or primer can be derived from the sense or anti-sense strand of a nucleic acid which encodes a tetratricopeptide repeat domain, e.g., a tetratricopeptide domain of human 55562, e.g., about amino acid residues 40 to 73 of SEQ ID NO:67, or a PD314595 homology domain, e.g., a PD314595 homology domain of human 55562, e.g., about amino acid residues 40 to 266 of SEQ ID NO:67.

In another embodiment a set of primers is provided, e.g., primers suitable for use in a PCR, which can be used to amplify a selected region of a 21617 or 55562 sequence, e.g., a domain, region, site or other sequence described herein. The primers should be at least 5, 10, or 50 base pairs in length and less than 100, or less than 200, base pairs in length. The primers should be identical, or differs by one base from a sequence disclosed herein or from a naturally occurring variant. For example, primers suitable for amplifying all or a portion of any of the following regions are provided: a tetratricopeptide repeat domain, e.g., a tetratricopeptide domain of human 55562, e.g., about amino acid residues 40 to 73 of SEQ ID NO:67, or a PD314595 homology domain, e.g., a PD314595 homology domain of human 55562, e.g., about amino acid residues 40 to 266 of SEQ ID NO:67.

A nucleic acid fragment can encode an epitope bearing region of a polypeptide described herein.

A nucleic acid fragment encoding a “biologically active portion of a 55562 polypeptide” can be prepared by isolating a portion of the nucleotide sequence of SEQ ID NO:66 or SEQ ID NO:68, which encodes a polypeptide having a 55562 biological activity (e.g., the biological activities of the 55562 proteins are described herein), expressing the encoded portion of the 55562 protein (e.g., by recombinant expression in vitro) and assessing the activity of the encoded portion of the 55562 protein. For example, a nucleic acid fragment encoding a biologically active portion of 21617 or 55562 includes a tetratricopeptide repeat domain, e.g., amino acid residues about 40 to 73 of SEQ ID NO:67. A nucleic acid fragment encoding a biologically active portion of a 55562 polypeptide may comprise a nucleotide sequence which is greater than 240, 275, 300, 331, or more nucleotides in length.

In preferred embodiments, a nucleic acid includes a nucleotide sequence which is about 300, 400, 500, 600, 700, 800, 900, 1000, 1100, 1200, 1300, or more nucleotides in length and hybridizes under a stringency condition described herein to a nucleic acid molecule of SEQ ID NO:66 or SEQ ID NO:68.

In a preferred embodiment, a nucleic acid fragment differs by at least 1, 2, 3, 10, 20, or more nucleotides from the sequence of Genbank accession number AC092587 or AL161992. In some preferred embodiments, a nucleic acid fragment differs by at least 1, 2, 3, 10, 20, or more nucleotides from a sequence disclosed in one or more of WO 01/70979 (e.g., SEQ ID NO:17485), WO 01/22920 (e.g., SEQ ID NO:338), EP 1033401 (e.g., SEQ ID NO:13112), WO 00/55174 (e.g., SEQ ID NO:213), and WO 01/57182 (e.g., SEQ ID NO:33456). Differences can include differing in length or sequence identity. For example, a nucleic acid fragment can: include one or more nucleotides from SEQ ID NO:66 located outside the region of nucleotides 401 to 594 and 672 to 1001; not include all of the nucleotides of Genbank accession number AC092587 or AL161992 and a sequence disclosed in one or more of WO 01/70979, WO 01/22920, EP 1033401, WO 00/55174, and WO 01/57182; or can differ by one or more nucleotides in the region of overlap.

21617 or 55562 Nucleic Acid Variants

The invention further encompasses nucleic acid molecules that differ from the nucleotide sequence shown in SEQ ID NO:63, SEQ ID NO:65, SEQ ID NO:66, or SEQ ID NO:68. Such differences can be due to degeneracy of the genetic code (and result in a nucleic acid which encodes the same 21617 or 55562 proteins as those encoded by the nucleotide sequence disclosed herein. In another embodiment, an isolated nucleic acid molecule of the invention has a nucleotide sequence encoding a protein having an amino acid sequence which differs, by at least 1, but less than 5, 10, 20, 50, or 100 amino acid residues that shown in SEQ ID NO:64 or SEQ ID NO:67. If alignment is needed for this comparison the sequences should be aligned for maximum homology. The encoded protein can differ by no more than 5, 4, 3, 2, or 1 amino acid. “Looped” out sequences from deletions or insertions, or mismatches, are considered differences.

Nucleic acids of the inventor can be chosen for having codons, which are preferred, or non-preferred, for a particular expression system. E.g., the nucleic acid can be one in which at least one codon, at preferably at least 10%, or 20% of the codons has been altered such that the sequence is optimized for expression in E. coli, yeast, human, insect, or CHO cells.

Nucleic acid variants can be naturally occurring, such as allelic variants (same locus), homologs (different locus), and orthologs (different organism) or can be non naturally occurring.

Non-naturally occurring variants can be made by mutagenesis techniques, including those applied to polynucleotides, cells, or organisms. The variants can contain nucleotide substitutions, deletions, inversions and insertions. Variation can occur in either or both the coding and non-coding regions. The variations can produce both conservative and non-conservative amino acid substitutions (as compared in the encoded product).

In a preferred embodiment, the nucleic acid differs from that of SEQ ID NO:63, SEQ ID NO:65, SEQ ID NO:66, or SEQ ID NO:68, e.g., as follows: by at least one but less than 10, 20, 30, or 40 nucleotides; at least one but less than 1%, 5%, 10% or 20% of the nucleotides in the subject nucleic acid. The nucleic acid can differ by no more than 5, 4, 3, 2, or 1 nucleotide. If necessary for this analysis the sequences should be aligned for maximum homology. “Looped” out sequences from deletions or insertions, or mismatches, are considered differences.

Orthologs, homologs, and allelic variants can be identified using methods known in the art. These variants comprise a nucleotide sequence encoding a polypeptide that is 50%, at least about 55%, typically at least about 70-75%, more typically at least about 80-85%, and most typically at least about 90-95% or more identical to the nucleotide sequence shown in SEQ ID NO:64 or SEQ ID NO:67 or a fragment of this sequence. Such nucleic acid molecules can readily be identified as being able to hybridize under a stringency condition described herein, to the nucleotide sequence shown in SEQ ID NO:64 or SEQ ID NO:67, or a fragment of the sequence. Nucleic acid molecules corresponding to orthologs, homologs, and allelic variants of the 21617 or 55562 cDNAs of the invention can further be isolated by mapping to the same chromosome or locus as the 21617 or 55562 gene.

Preferred 21617 variants include those that are correlated with the ability to oxidize an alcohol group or reduce a carbonyl group present in a small molecule, e.g., a steroid, lipid, toxin, or xenobiotic compound, or a protein substrate.

Allelic variants of 21617, e.g., human 21617, include both functional and non-functional proteins. Functional allelic variants are naturally occurring amino acid sequence variants of the 21617 protein within a population that maintain the ability to bind a substrate, e.g., an alcohol or steroid. Functional allelic variants will typically contain only conservative substitution of one or more amino acids of SEQ ID NO:64, or substitution, deletion or insertion of non-critical residues in non-critical regions of the protein. Non-functional allelic variants are naturally-occurring amino acid sequence variants of the 21617, e.g., human 21617, protein within a population that do not have the ability to bind or otherwise act upon a substrate. Non-functional allelic variants will typically contain a non-conservative substitution, a deletion, or insertion, or premature truncation of the amino acid sequence of SEQ ID NO:64, or a substitution, insertion, or deletion in critical residues or critical regions of the protein.

Preferred 55562 variants include those that are correlated with either the ability to stimulate the formation of a complex, e.g., with other tetratricopeptide repeat containing molecules or, conversely, the ability to disrupt the formation of such a complex.

Allelic variants of 55562, e.g., human 21617 or 55562, include both functional and non-functional proteins. Functional allelic variants are naturally occurring amino acid sequence variants of the 21617 or 55562 protein within a population that maintain the ability to bind to human 55562-interacting proteins. Functional allelic variants will typically contain only conservative substitution of one or more amino acids of SEQ ID NO:67, or substitution, deletion or insertion of non-critical residues in non-critical regions of the protein. Non-functional allelic variants are naturally-occurring amino acid sequence variants of the 55562, e.g., human 55562, protein within a population that do not have the ability human 55562-interacting proteins. Non-functional allelic variants will typically contain a non-conservative substitution, a deletion, or insertion, or premature truncation of the amino acid sequence of SEQ ID NO:67, or a substitution, insertion, or deletion in critical residues or critical regions of the protein.

Moreover, nucleic acid molecules encoding other 21617 or 55562 family members and, thus, which have a nucleotide sequence which differs from the 21617 or 55562 sequences of SEQ ID NO:63, SEQ ID NO:65, SEQ ID NO:66, or SEQ ID NO:68 are intended to be within the scope of the invention.

Antisense Nucleic Acid Molecules, Ribozymes and Modified 21617 or 55562 Nucleic Acid Molecules

In another aspect, the invention features, an isolated nucleic acid molecule which is antisense to 21617 or 55562. An “antisense” nucleic acid can include a nucleotide sequence which is complementary to a “sense” nucleic acid encoding a protein, e.g., complementary to the coding strand of a double-stranded cDNA molecule or complementary to an mRNA sequence. The antisense nucleic acid can be complementary to an entire 21617 or 55562 coding strand, or to only a portion thereof (e.g., the coding region of human 21617 or 55562 corresponding to SEQ ID NO:65 and SEQ ID NO:68, respectively). In another embodiment, the antisense nucleic acid molecule is antisense to a “noncoding region” of the coding strand of a nucleotide sequence encoding 21617 or 55562 (e.g., the 5′ and 3′ untranslated regions).

An antisense nucleic acid can be designed such that it is complementary to the entire coding region of 21617 or 55562 mRNA, but more preferably is an oligonucleotide which is antisense to only a portion of the coding or noncoding region of 21617 or 55562 mRNA. For example, the antisense oligonucleotide can be complementary to the region surrounding the translation start site of 21617 or 55562 mRNA, e.g., between the −10 and +10 regions of the target gene nucleotide sequence of interest. An antisense oligonucleotide can be, for example, about 7, 10, 15, 20, 25, 30, 35, 40, 45, 50, 55, 60, 65, 70, 75, 80, or more nucleotides in length.

An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. The antisense nucleic acid also can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject (e.g., by direct injection at a tissue site), or generated in situ such that they hybridize with or bind to cellular mRNA and/or genomic DNA encoding a 21617 or 55562 protein to thereby inhibit expression of the protein, e.g., by inhibiting transcription and/or translation. Alternatively, antisense nucleic acid molecules can be modified to target selected cells and then administered systemically. For systemic administration, antisense molecules can be modified such that they specifically bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids. Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

In still another embodiment, an antisense nucleic acid of the invention is a ribozyme. A ribozyme having specificity for a 21617 or 55562-encoding nucleic acid can include one or more sequences complementary to the nucleotide sequence of a 21617 or 55562 cDNA disclosed herein (i.e., SEQ ID NO:63, SEQ ID NO:65, SEQ ID NO:66, or SEQ ID NO:68), and a sequence having known catalytic sequence responsible for mRNA cleavage (see U.S. Pat. No. 5,093,246 or Haselhoff and Gerlach (1988) Nature 334:585-591). For example, a derivative of a Tetrahymena L-19 IVS RNA can be constructed in which the nucleotide sequence of the active site is complementary to the nucleotide sequence to be cleaved in a 21617 or 55562-encoding mRNA. See, e.g., Cech et al. U.S. Pat. No. 4,987,071; and Cech et al. U.S. Pat. No. 5,116,742. Alternatively, 21617 or 55562 mRNA can be used to select a catalytic RNA having a specific ribonuclease activity from a pool of RNA molecules. See, e.g., Bartel, D. and Szostak, J. W. (1993) Science 261:1411-1418. 21617 or 55562 gene expression can be inhibited by targeting nucleotide sequences complementary to the regulatory region of the 21617 or 55562 (e.g., the 21617 or 55562 promoter and/or enhancers) to form triple helical structures that prevent transcription of the 21617 or 55562 gene in target cells. See generally, Helene, C. (1991)Anticancer Drug Des. 6:569-84; Helene, C. i (1992) Ann. N.Y. Acad. Sci. 660:27-36; and Maher, L. J. (1992) Bioassays 14:807-15. The potential sequences that can be targeted for triple helix formation can be increased by creating a so-called “switchback” nucleic acid molecule. Switchback molecules are synthesized in an alternating 5′-3′, 3′-5′ manner, such that they base pair with first one strand of a duplex and then the other, eliminating the necessity for a sizeable stretch of either purines or pyrimidines to be present on one strand of a duplex.

The invention also provides detectably labeled oligonucleotide primer and probe molecules. Typically, such labels are chemiluminescent, fluorescent, radioactive, or colorimetric.

A 21617 or 55562 nucleic acid molecule can be modified at the base moiety, sugar moiety or phosphate backbone to improve, e.g., the stability, hybridization, or solubility of the molecule. For non-limiting examples of synthetic oligonucleotides with modifications see Toulmé (2001) Nature Biotech. 19:17 and Faria et al. (2001) Nature Biotech. 19:40-44. Such phosphoramidite oligonucleotides can be effective antisense agents.

For example, the deoxyribose phosphate backbone of the nucleic acid molecules can be modified to generate peptide nucleic acids (see Hyrup B. et al. (1996) Bioorganic & Medicinal Chemistry 4: 5-23). As used herein, the terms “peptide nucleic acid” or “PNA” refers to a nucleic acid mimic, e.g., a DNA mimic, in which the deoxyribose phosphate backbone is replaced by a pseudopeptide backbone and only the four natural nucleobases are retained. The neutral backbone of a PNA can allow for specific hybridization to DNA and RNA under conditions of low ionic strength. The synthesis of PNA oligomers can be performed using standard solid phase peptide synthesis protocols as described in Hyrup B. et al. (1996) supra and Perry-O'Keefe et al. Proc. Natl. Acad. Sci. 93: 14670-675.

PNAs of 21617 or 55562 nucleic acid molecules can be used in therapeutic and diagnostic applications. For example, PNAs can be used as antisense or antigene agents for sequence-specific modulation of gene expression by, for example, inducing transcription or translation arrest or inhibiting replication. PNAs of 21617 or 55562 nucleic acid molecules can also be used in the analysis of single base pair mutations in a gene, (e.g., by PNA-directed PCR clamping); as ‘artificial restriction enzymes’ when used in combination with other enzymes, (e.g., S1 nucleases (Hyrup B. et al. (1996) supra)); or as probes or primers for DNA sequencing or hybridization (Hyrup B. et al. (1996) supra; Perry-O'Keefe supra).

In other embodiments, the oligonucleotide may include other appended groups such as peptides (e.g., for targeting host cell receptors in vivo), or agents facilitating transport across the cell membrane (see, e.g., Letsinger et al. (1989) Proc. Natl. Acad. Sci. USA 86:6553-6556; Lemaitre et al. (1987) Proc. Natl. Acad. Sci. USA 84:648-652; PCT Publication No. WO88/09810) or the blood-brain barrier (see, e.g., PCT Publication No. WO89/10134). In addition, oligonucleotides can be modified with hybridization-triggered cleavage agents (see, e.g., Krol et al. (1988) Bio-Techniques 6:958-976) or intercalating agents. (see, e.g., Zon (1988) Pharm. Res. 5:539-549). To this end, the oligonucleotide maybe conjugated to another molecule, (e.g., a peptide, hybridization triggered cross-linking agent, transport agent, or hybridization-triggered cleavage agent).

The invention also includes molecular beacon oligonucleotide primer and probe molecules having at least one region which is complementary to a 21617 or 55562 nucleic acid of the invention, two complementary regions one having a fluorophore and one a quencher such that the molecular beacon is useful for quantitating the presence of the 21617 or 55562 nucleic acid of the invention in a sample. Molecular beacon nucleic acids are described, for example, in Lizardi et al., U.S. Pat. No. 5,854,033; Nazarenko et al., U.S. Pat. No. 5,866,336, and Livak et al., U.S. Pat. No. 5,876,930.

Isolated 21617 Polypeptides

In another aspect, the invention features an isolated 21617 protein or fragments thereof, e.g., biologically active portions, for use as immunogens or antigens to raise or test (or more generally to bind) anti-21617 antibodies. 21617 protein can be isolated from cells or tissue sources using standard protein purification techniques. 21617 protein or fragments thereof can be produced by recombinant DNA techniques or synthesized chemically.

Polypeptides of the invention include those which arise as a result of the existence of multiple genes, alternative transcription events, alternative RNA splicing events, and alternative translational and post-translational events. The polypeptide can be expressed in systems, e.g., cultured cells, which result in substantially the same post-translational modifications present when expressed the polypeptide is expressed in a native cell, or in systems which result in the alteration or omission of post-translational modifications, e.g., glycosylation or cleavage, present when expressed in a native cell.

In a preferred embodiment, a 21617 polypeptide has one or more of the following characteristics:

(i) it has the ability to oxidize an alcohol group on a substrate molecule;

(ii) it has the ability to reduce a carbonyl group on a substrate molecule;

(iii) it has the ability bind a co-enzyme;

(iv) it participates in the metabolism of a substrate, e.g., a small molecule substrate, e.g., an alcohol, steroid, or fatty acid molecule;

(v) it has a molecular weight, e.g., a deduced molecular weight (preferably ignoring any contribution of post translational modifications), amino acid composition, or other physical characteristic of a 21617 polypeptide, e.g., the protein described in SEQ ID NO:64;

(vi) it has an overall sequence similarity of at least 60%, more preferably at least 70%, 80%, 90%, or 95%, with a polypeptide of SEQ ID NO:64; or

(vii) it has a short chain dehydrogenase domain which is preferably about 70%, 80%, 90%, 95%, 98%, 99%, or more homologous with amino acid residues about 37 to 249 of SEQ ID NO:64;

(viii) it can be found in a tumor cell or tissue, e.g., a colon, colorectal, breast, lung, cervical, or liver tumor cell or tissue;

(ix) it has a dehydrogenase family signature motif (PS00061);

(x) it has a predicted signal peptide;

(xi) it has at least one, preferable two dileucine motifs;

(xii) it has at least one predicted glycosaminoglycan attachment site (PS00002);

(xiii) it has at least one, two, preferably three predicted Protein Kinase C phosphorylation sites (PS00005);

(xiv) it has at least one, preferable two predicted Casein Kinase II phosphorylation sites (PS00006);

(xv) it has at least one, two, three, four, five, preferably six predicted N-myristoylation sites (PS00008); and

(xvi) it has at least one predicted amidation site (PS00009).

In a preferred embodiment the 21617 protein, or fragment thereof, differs from the corresponding sequence in SEQ ID NO:64. In one embodiment it differs by at least one but by less than 15, 10 or 5 amino acid residues. In another it differs from the corresponding sequence in SEQ ID NO:64 by at least one residue but less than 20%, 15%, 10% or 5% of the residues in it differ from the corresponding sequence in SEQ ID NO:64. (If this comparison requires alignment the sequences should be aligned for maximum homology. “Looped” out sequences from deletions or insertions, or mismatches, are considered differences.) The differences are, preferably, differences or changes at a non essential residue or a conservative substitution. In a preferred embodiment the differences are not in the short chain dehydrogenase domain. For example, the differences are in the region of amino acid residues 1-36 and 250-341 of SEQ ID NO:64. In another preferred embodiment one or more differences are in the short chain dehydrogenase domain, e.g., residues 37-249 of SEQ ID NO:64.

Other embodiments include a protein that contain one or more changes in amino acid sequence, e.g., a change in an amino acid residue which is not essential for activity. Such 21617 proteins differ in amino acid sequence from SEQ ID NO:64, yet retain biological activity.

In one embodiment, the protein includes an amino acid sequence at least about 60%, 65%, 70%, 75%, 80%, 85%, 90%, 95%, 98%, 99%, or more homologous to SEQ ID NO:64.

A 21617 protein or fragment is provided which varies from the sequence of SEQ ID NO:64 in regions defined by amino acids about 1-36 and 250-341 by at least one but by less than 15, 10 or 5 amino acid residues in the protein or fragment but which does not differ from SEQ ID NO:64 in regions defined by amino acids about 37-249 of SEQ ID NO:64. (If this comparison requires alignment the sequences should be aligned for maximum homology. “Looped” out sequences from deletions or insertions, or mismatches, are considered differences.) In some embodiments the difference is at a non-essential residue or is a conservative substitution, while in others the difference is at an essential residue or is a non-conservative substitution.

In one embodiment, a biologically active portion of a 21617 protein includes a short chain dehydrogenase domain. Moreover, other biologically active portions, in which other regions of the protein are deleted, can be prepared by recombinant techniques and evaluated for one or more of the functional activities of a native 21617 protein.

In a preferred embodiment, the 21617 protein has an amino acid sequence shown in SEQ ID NO:64. In other embodiments, the 21617 protein is substantially identical to SEQ ID NO:64. In yet another embodiment, the 21617 protein is substantially identical to SEQ ID NO:64 and retains the functional activity of the protein of SEQ ID NO:64, as described in detail in the subsections above.

In a preferred embodiment, a fragment differs by at least 1, 2, 3, 10, 20, or more amino acid residues encoded by a sequence present in Genbank accession number AF286885, G28278, G29385, or G25914 or a sequence disclosed in one or more of WO 00/09552, WO 01/51628, WO 01/55301, WO 01/55364, WO 01/77289, WO 01/5719, EP1033401 or WO 01/02568. Differences can include differing in length or sequence identity. For example, a fragment can: include one or more amino acid residues from SEQ ID NO:64 outside the region encoded by nucleotides 475-3607; not include all of the amino acid residues encoded by a nucleotide sequence in Genbank accession number AF286885, G28278, G29385, or G25914, or a sequence disclosed in WO 00/09552, WO 01/51628, WO 01/55301, WO 01/55364, WO 01/77289, WO 01/5719, EP1033401 or WO 01/02568, e.g., can be one or more amino acid residues shorter (at one or both ends) than a sequence encoded by the nucleotide sequence in Genbank accession number AF286885, G28278, G29385, or G25914 or a sequence disclosed in one or more of WO 00/09552, WO 01/51628, WO 01/55301, WO 01/55364, WO 01/77289, WO 01/5719, EP1033401 or WO 01/02568; or can differ by one or more amino acid residues in the region of overlap.

Isolated 55562 Polypeptides

In another aspect, the invention features an isolated 5562 protein or fragments thereof, e.g., biologically active portions, for use as immunogens or antigens to raise or test (or more generally to bind) anti-55562 antibodies. 55562 protein can be isolated from cells or tissue sources using standard protein purification techniques. 55562 protein or fragments thereof can be produced by recombinant DNA techniques or synthesized chemically.

Polypeptides of the invention include those that arise as a result of the existence of multiple genes, alternative transcription events, alternative RNA splicing events, and alternative translational and post-translational events. The polypeptide can be expressed in systems, e.g., cultured cells, which result in substantially the same post-translational modifications present when expressed the polypeptide is expressed in a native cell, or in systems which result in the alteration or omission of post-translational modifications, e.g., glycosylation or cleavage, present when expressed in a native cell.

In a preferred embodiment, a 55562 polypeptide has one or more of the following characteristics:

(i) it has the ability to facilitate the formation of a protein complex, e.g., a protein complex containing at least one other tetratricopeptide repeat containing protein;

(ii) it has a molecular weight, e.g., a deduced molecular weight (preferably ignoring any contribution of post translational modifications), amino acid composition, or other physical characteristic of a 55562 polypeptide, e.g., the protein of SEQ ID NO:67;

(iii) it has an overall sequence similarity of at least 60%, more preferably at least 70%, 80%, 90%, 95%, 98%, 99%, or more with a polypeptide of SEQ ID NO:67;

(iv) it has a tetratricopeptide repeat domain which is preferably about 70%, 80%, 90%, 95%, 98%, 99%, or more homologous with amino acid residues about 40 to 73 of SEQ ID NO:67;

(v) it has conserved TPR features, e.g., an alanine corresponding to residue 47 of SEQ ID NO:67, an alanine corresponding to residue 58 of SEQ ID NO:67; and a tyrosine corresponding to residue 55 of SEQ ID NO:67;

(vi) it has a PD314595 homology domain which is preferably about 70%, 80%, 90%, 95%, 98%, 99%, or more homologous with amino acid residues about 40 to 266 of SEQ ID NO:67;

(vii) it has the ability to modulate intracellular transport;

(viii) it has the ability to modulate an enzyme activity, e.g., in response to a second messenger;

(viii) it has at least one, two three, preferably four predicted Protein Kinase C phosphorylation sites (PS00005);

(ix) it has at least one, two, three, preferably four predicted Casein Kinase II phosphorylation sites (PS00006);

(x) it has at least one, two, preferably three predicted cAMP/cGMP-dependent protein kinase phosphorylation sites (PS00004;

(xi) it has at least one, preferably two predicted N-glycosylation sites (PS00001); or

(xii) it has at least one predicted N-myristylation sites (PS00008).

In a preferred embodiment the 55562 protein, or fragment thereof, differs from the corresponding sequence in SEQ ID NO:67. In one embodiment it differs by at least one but by less than 15, 10 or 5 amino acid residues. In another it differs from the corresponding sequence in SEQ ID NO:67 by at least one residue but less than 20%, 15%, 10% or 5% of the residues in it differ from the corresponding sequence in SEQ ID NO:67. (If this comparison requires alignment the sequences should be aligned for maximum homology. “Looped” out sequences from deletions or insertions, or mismatches, are considered differences.) The differences are, preferably, differences or changes at a non-essential residue or a conservative substitution. In a preferred embodiment the differences are not in the tetratricopeptide repeat domain. In another preferred embodiment one or more differences are in the tetratricopeptide repeat domain.

Other embodiments include a protein that contain one or more changes in amino acid sequence, e.g., a change in an amino acid residue which is not essential for activity. Such 55562 proteins differ in amino acid sequence from SEQ ID NO:67, yet retain biological activity.

In one embodiment, the protein includes an amino acid sequence at least about 60%, 65%, 70%, 75%, 80%, 85%, 90%, 95%, 98%, 99%, or more homologous to SEQ ID NO:67.

A 55562 protein or fragment is provided which varies from the sequence of SEQ ID ID NO:5 in regions defined by amino acids about 1 to 39 and 74 to 274 by at least one but by less than 15, 10 or 5 amino acid residues in the protein or fragment but which does not differ from SEQ ID NO:67 in regions defined by amino acids about 40 to 73 of SEQ ID ID NO:5. (If this comparison requires alignment the sequences should be aligned for maximum homology. “Looped” out sequences from deletions or insertions, or mismatches, are considered differences.) In some embodiments the difference is at a non-essential residue or is a conservative substitution, while in others the difference is at an essential residue or is a non-conservative substitution.

In one embodiment, a biologically active portion of a 55562 protein includes a tetratricopeptide repeat domain. Moreover, other biologically active portions, in which other regions of the protein are deleted, can be prepared by recombinant techniques and evaluated for one or more of the functional activities of a native 55562 protein.

In a preferred embodiment, the 55562 protein has an amino acid sequence shown in SEQ ID NO:67. In other embodiments, the 55562 protein is substantially identical to SEQ ID NO:67. In yet another embodiment, the 55562 protein is substantially identical to SEQ ID NO:67 and retains the functional activity of the protein of SEQ ID NO:67, as described in detail in the subsections above.

In a preferred embodiment, a 55562 protein fragment differs by at least 1, 2, 3, 10, 20, or more amino acids from protein sequence encoded by the sequence of Genbank accession number AC092587, AL161992, or AK005342. In some preferred embodiments, a 55562 protein fragment differs by at least 1, 2, 3, 10, 20, or more amino acids from a protein sequence encoded by a sequence disclosed in one or more of WO 01/70979 (e.g., SEQ ID NO:17485), WO 01/22920 (e.g., SEQ ID NO:338), EP 1033401 (e.g., SEQ ID NO:13112), WO 00/55174 (e.g., SEQ ID NO:213), and WO 01/57182 (e.g., SEQ ID NO:33456). Differences can include differing in length or sequence identity. For example, a 55562 protein fragment can: include one or more amino acid residues of SEQ ID NO:67 located outside the region encoded by nucleotides 401 to 594 and 672 to 1001 of SEQ ID NO:66; not include all of the amino acid encoded by the nucleic acid molecules of Genbank accession number AC092587, AL161992 or AK005342, or a sequence disclosed in one or more of WO 01/70979, WO 01/22920, EP 1033401, WO 00/55174, and WO 01/57182, e.g., can be one or more amino acid residues shorter (at one or both ends) than a sequence encoded by the nucleotide sequence in Genbank accession number AC092587, AL161992 or AK005342, or a sequence disclosed in one or more of WO 01/70979, WO 01/22920, EP 1033401, WO 00/55174, and WO 01/57182; or can differ by one or more amino acid in the region of overlap.

21617 or 55562 Chimeric or Fusion Proteins

In another aspect, the invention provides 21617 or 55562 chimeric or fusion proteins. As used herein, a 21617 or 55562 “chimeric protein” or “fusion protein” includes a 21617 or 55562 polypeptide linked to a non-21617 or non-55562 polypeptide, respectively. A “non-21617 polypeptide” or “non-55562 polypeptide” refers to a polypeptide having an amino acid sequence corresponding to a protein which is not substantially homologous to the 21617 or 55562 protein, e.g., a protein which is different from the 21617 or 55562 protein and which is derived from the same or a different organism. The 21617 or 55562 polypeptide of the fusion protein can correspond to all or a portion e.g., a fragment described herein of a 21617 or 55562 amino acid sequence. In a preferred embodiment, a 21617 or 55562 fusion protein includes at least one (or two) biologically active portion of a 21617 or 55562 protein. The non-21617 or 55562 polypeptide can be fused to the N-terminus or C-terminus of the 21617 or 55562 polypeptide.

The fusion protein can include a moiety which has a high affinity for a ligand. For example, the fusion protein can be a GST-21617 or 55562 fusion protein in which the 21617 or 55562 sequences are fused to the C-terminus of the GST sequences. Such fusion proteins can facilitate the purification of recombinant 21617 or 55562. Alternatively, the fusion protein can be a 21617 or 55562 protein containing a heterologous signal sequence at its N-terminus. In certain host cells (e.g., mammalian host cells), expression and/or secretion of 21617 or 55562 can be increased through use of a heterologous signal sequence.

Fusion proteins can include all or a part of a serum protein, e.g., an IgG constant region, or human serum albumin.

The 21617 or 55562 fusion proteins of the invention can be incorporated into pharmaceutical compositions and administered to a subject in vivo. The 21617 or 55562 fusion proteins can be used to affect the bioavailability of a 21617 or 55562 substrate. 21617 or 55562 fusion proteins may be useful therapeutically for the treatment of disorders caused by, for example, (i) aberrant modification or mutation of a gene encoding a 21617 or 55562 protein; (ii) mis-regulation of the 21617 or 55562 gene; and (iii) aberrant post-translational modification of a 21617 or 55562 protein.

Moreover, the 21617 or 55562-fusion proteins of the invention can be used as immunogens to produce anti-21617 or 55562 antibodies in a subject, to purify 21617 or 55562 ligands and in screening assays to identify molecules which inhibit the interaction of 21617 or 55562 with a 21617 or 55562 substrate.

Expression vectors are commercially available that already encode a fusion moiety (e.g., a GST polypeptide). A 21617 or 55562-encoding nucleic acid can be cloned into such an expression vector such that the fusion moiety is linked in-frame to the 21617 or 55562 protein.

Variants of 21617 or 55562 Proteins

In another aspect, the invention also features a variant of a 21617 or 55562 polypeptide, e.g., which functions as an agonist (mimetics) or as an antagonist. Variants of the 21617 or 55562 proteins can be generated by mutagenesis, e.g., discrete point mutation, the insertion or deletion of sequences or the truncation of a 21617 or 55562 protein. An agonist of the 21617 or 55562 proteins can retain substantially the same, or a subset, of the biological activities of the naturally occurring form of a 21617 or 55562 protein. An antagonist of a 21617 or 55562 protein can inhibit one or more of the activities of the naturally occurring form of the 21617 or 55562 protein by, for example, competitively modulating a 21617 or 55562-mediated activity of a 21617 or 55562 protein. Thus, specific biological effects can be elicited by treatment with a variant of limited function. Preferably, treatment of a subject with a variant having a subset of the biological activities of the naturally occurring form of the protein has fewer side effects in a subject relative to treatment with the naturally occurring form of the 21617 or 55562 protein.

Variants of a 21617 or 55562 protein can be identified by screening combinatorial libraries of mutants, e.g., truncation mutants, of a 21617 or 55562 protein for agonist or antagonist activity.

Libraries of fragments e.g., N-terminal, C-terminal, or internal fragments, of a 21617 or 55562 protein coding sequence can be used to generate a variegated population of fragments for screening and subsequent selection of variants of a 21617 or 55562 protein. Variants in which a cysteine residues is added or deleted or in which a residue which is glycosylated is added or deleted are particularly preferred.

Methods for screening gene products of combinatorial libraries made by point mutations or truncation, and for screening cDNA libraries for gene products having a selected property are known in the art. Such methods are adaptable for rapid screening of the gene libraries generated by combinatorial mutagenesis of 21617 or 55562 proteins. Recursive ensemble mutagenesis (REM), a new technique which enhances the frequency of functional mutants in the libraries, can be used in combination with the screening assays to identify 21617 or 55562 variants (Arkin and Yourvan (1992) Proc. Natl. Acad. Sci. USA 89:7811-7815; Delgrave et al. (1993) Protein Engineering 6:327-331).

Cell based assays can be exploited to analyze a variegated 21617 or 55562 library. For example, a library of expression vectors can be transfected into a cell line, e.g., a cell line, which ordinarily responds to 21617 or 55562 in a substrate-dependent manner. The transfected cells are then contacted with 21617 or 55562 and the effect of the expression of the mutant on signaling by the 21617 or 55562 substrate can be detected, e.g., by measuring an activity associated with 21617 or 55562 expression, e.g., cellular proliferation and/or differentiation. Plasmid DNA can then be recovered from the cells which score for inhibition, or alternatively, potentiation of signaling by the 21617 or 55562 substrate, and the individual clones further characterized.

In another aspect, the invention features a method of making a 21617 or 55562 polypeptide, e.g., a peptide having a non-wild type activity, e.g., an antagonist, agonist, or super agonist of a naturally occurring 21617 or 55562 polypeptide, e.g., a naturally occurring 21617 or 55562 polypeptide. The method includes: altering the sequence of a 21617 or 55562 polypeptide, e.g., altering the sequence, e.g., by substitution or deletion of one or more residues of a non-conserved region, a domain or residue disclosed herein, and testing the altered polypeptide for the desired activity.

In another aspect, the invention features a method of making a fragment or analog of a 21617 or 55562 polypeptide a biological activity of a naturally occurring 21617 or 55562 polypeptide. The method includes: altering the sequence, e.g., by substitution or deletion of one or more residues, of a 21617 or 55562 polypeptide, e.g., altering the sequence of a non-conserved region, or a domain or residue described herein, and testing the altered polypeptide for the desired activity.

Anti-21617 or 55562 Antibodies

In another aspect, the invention provides an anti-21617 or 55562 antibody, or a fragment thereof (e.g., an antigen-binding fragment thereof). The term “antibody” as used herein refers to an immunoglobulin molecule or immunologically active portion thereof, i.e., an antigen-binding portion. As used herein, the term “antibody” refers to a protein comprising at least one, and preferably two, heavy (H) chain variable regions (abbreviated herein as VH), and at least one and preferably two light (L) chain variable regions (abbreviated herein as VL). The VH and VL regions can be further subdivided into regions of hypervariability, termed “complementarity determining regions” (“CDR”), interspersed with regions that are more conserved, termed “framework regions” (FR). The extent of the framework region and CDR's has been precisely defined (see, Kabat, E. A., et al. (1991) Sequences of Proteins of Immunological Interest, Fifth Edition, U.S. Department of Health and Human Services, NIH Publication No. 91-3242, and Chothia, C. et al. (1987) J. Mol. Biol. 196:901-917, which are incorporated herein by reference). Each VH and VL is composed of three CDR's and four FRs, arranged from amino-terminus to carboxy-terminus in the following order: FR1, CDR1, FR2, CDR2, FR3, CDR3, FR4.

The anti-21617 or 55562 antibody can further include a heavy and light chain constant region, to thereby form a heavy and light immunoglobulin chain, respectively. In one embodiment, the antibody is a tetramer of two heavy immunoglobulin chains and two light immunoglobulin chains, wherein the heavy and light immunoglobulin chains are inter-connected by, e.g., disulfide bonds. The heavy chain constant region is comprised of three domains, CH1, CH2 and CH3. The light chain constant region is comprised of one domain, CL. The variable region of the heavy and light chains contains a binding domain that interacts with an antigen. The constant regions of the antibodies typically mediate the binding of the antibody to host tissues or factors, including various cells of the immune system (e.g., effector cells) and the first component (C1q) of the classical complement system.

As used herein, the term “immunoglobulin” refers to a protein consisting of one or more polypeptides substantially encoded by immunoglobulin genes. The recognized human immunoglobulin genes include the kappa, lambda, alpha (IgA1 and IgA2), gamma (IgG1, IgG2, IgG3, IgG4), delta, epsilon and mu constant region genes, as well as the myriad immunoglobulin variable region genes. Full-length immunoglobulin “light chains” (about 25 KDa or 214 amino acids) are encoded by a variable region gene at the NH2-terminus (about 110 amino acids) and a kappa or lambda constant region gene at the COOH—terminus. Full-length immunoglobulin “heavy chains” (about 50 KDa or 446 amino acids), are similarly encoded by a variable region gene (about 116 amino acids) and one of the other aforementioned constant region genes, e.g., gamma (encoding about 330 amino acids).

The term “antigen-binding fragment” of an antibody (or simply “antibody portion,” or “fragment”), as used herein, refers to one or more fragments of a full-length antibody that retain the ability to specifically bind to the antigen, e.g., 21617 or 55562 polypeptide or fragment thereof. Examples of antigen-binding fragments of the anti-21617 or 55562 antibody include, but are not limited to: (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also encompassed within the term “antigen-binding fragment” of an antibody. These antibody fragments are obtained using conventional techniques known to those with skill in the art, and the fragments are screened for utility in the same manner as are intact antibodies.

The anti-21617 or 55562 antibody can be a polyclonal or a monoclonal antibody. In other embodiments, the antibody can be recombinantly produced, e.g., produced by phage display or by combinatorial methods.

Phage display and combinatorial methods for generating anti-21617 or 55562 antibodies are known in the art (as described in, e.g., Ladner et al. U.S. Pat. No. 5,223,409; Kang et al. International Publication No. WO 92/18619; Dower et al. International Publication No. WO 91/17271; Winter et al. International Publication WO 92/20791; Markland et al. International Publication No. WO 92/15679; Breitling et al. International Publication WO 93/01288; McCafferty et al. International Publication No. WO 92/01047; Garrard et al. International Publication No. WO 92/09690; Ladner et al. International Publication No. WO 90/02809; Fuchs et al. (1991) Bio/Technology 9:1370-1372; Hay et al. (1992) Hum Antibod Hybridomas 3:81-85; Huse et al. (1989) Science 246:1275-1281; Griffths et al. (1993) EMBO J. 12:725-734; Hawkins et al. (1992) J Mol Biol 226:889-896; Clackson et al. (1991) Nature 352:624-628; Gram et al. (1992) PNAS 89:3576-3580; Garrad et al. (1991) Bio/Technology 9:1373-1377; Hoogenboom et al. (1991) Nuc Acid Res 19:4133-4137; and Barbas et al. (1991) PNAS 88:7978-7982, the contents of all of which are incorporated by reference herein).

In one embodiment, the anti-21617 or 55562 antibody is a fully human antibody (e.g., an antibody made in a mouse which has been genetically engineered to produce an antibody from a human immunoglobulin sequence), or a non-human antibody, e.g., a rodent (mouse or rat), goat, primate (e.g., monkey), camel antibody. Preferably, the non-human antibody is a rodent (mouse or rat antibody). Method of producing rodent antibodies are known in the art.

Human monoclonal antibodies can be generated using transgenic mice carrying the human immunoglobulin genes rather than the mouse system. Splenocytes from these transgenic mice immunized with the antigen of interest are used to produce hybridomas that secrete human mAbs with specific affinities for epitopes from a human protein (see, e.g., Wood et al. International Application WO 91/00906, Kucherlapati et al. PCT publication WO 91/10741; Lonberg et al. International Application WO 92/03918; Kay et al. International Application 92/03917; Lonberg, N. et al. 1994 Nature 368:856-859; Green, L. L. et al. 1994 Nature Genet. 7:13-21; Morrison, S. L. et al. 1994 Proc. Natl. Acad. Sci. USA 81:6851-6855; Bruggeman et al. 1993 Year Immunol 7:33-40; Tuaillon et al. 1993 PNAS 90:3720-3724; Bruggeman et al. 1991 Eur J Immunol 21:1323-1326).

An anti-21617 or 55562 antibody can be one in which the variable region, or a portion thereof, e.g., the CDR's, are generated in a non-human organism, e.g., a rat or mouse. Chimeric, CDR-grafted, and humanized antibodies are within the invention. Antibodies generated in a non-human organism, e.g., a rat or mouse, and then modified, e.g., in the variable framework or constant region, to decrease antigenicity in a human are within the invention.

Chimeric antibodies can be produced by recombinant DNA techniques known in the art. For example, a gene encoding the Fc constant region of a murine (or other species) monoclonal antibody molecule is digested with restriction enzymes to remove the region encoding the murine Fe, and the equivalent portion of a gene encoding a human Fe constant region is substituted (see Robinson et al., International Patent Publication PCT/US86/02269; Akira, et al., European Patent Application 184,187; Taniguchi, M., European Patent Application 171,496; Morrison et al., European Patent Application 173,494; Neuberger et al., International Application WO 86/01533; Cabilly et al. U.S. Pat. No. 4,816,567; Cabilly et al., European Patent Application 125,023; Better et al. (1988 Science 240:1041-1043); Liu et al. (1987) PNAS 84:3439-3443; Liu et al., 1987, J. Immunol. 139:3521-3526; Sun et al. (1987) PNAS 84:214-218; Nishimura et al., 1987, Canc. Res. 47:999-1005; Wood et al. (1985) Nature 314:446-449; and Shaw et al., 1988, J. Natl Cancer Inst. 80:1553-1559).

A humanized or CDR-grafted antibody will have at least one or two but generally all three recipient CDR's (of heavy and or light immuoglobulin chains) replaced with a donor CDR. The antibody may be replaced with at least a portion of a non-human CDR or only some of the CDR's may be replaced with non-human CDR's. It is only necessary to replace the number of CDR's required for binding of the humanized antibody to a 21617 or 55562 or a fragment thereof. Preferably, the donor will be a rodent antibody, e.g., a rat or mouse antibody, and the recipient will be a human framework or a human consensus framework. Typically, the immunoglobulin providing the CDR's is called the “donor” and the immunoglobulin providing the framework is called the “acceptor.” In one embodiment, the donor immunoglobulin is a non-human (e.g., rodent). The acceptor framework is a naturally-occurring (e.g., a human) framework or a consensus framework, or a sequence about 85% or higher, preferably 90%, 95%, 99% or higher identical thereto.

As used herein, the term “consensus sequence” refers to the sequence formed from the most frequently occurring amino acids (or nucleotides) in a family of related sequences (See e.g., Winnaker, From Genes to Clones (Verlagsgesellschaft, Weinheim, Germany 1987). In a family of proteins, each position in the consensus sequence is occupied by the amino acid occurring most frequently at that position in the family. If two amino acids occur equally frequently, either can be included in the consensus sequence. A “consensus framework” refers to the framework region in the consensus immunoglobulin sequence.

An antibody can be humanized by methods known in the art. Humanized antibodies can be generated by replacing sequences of the Fv variable region which are not directly involved in antigen binding with equivalent sequences from human Fv variable regions. General methods for generating humanized antibodies are provided by Morrison, S. L., 1985, Science 229:1202-1207, by Oi et al., 1986, BioTechniques 4:214, and by Queen et al. U.S. Pat. No. 5,585,089, U.S. Pat. No. 5,693,761 and U.S. Pat. No. 5,693,762, the contents of all of which are hereby incorporated by reference. Those methods include isolating, manipulating, and expressing the nucleic acid sequences that encode all or part of immunoglobulin Fv variable regions from at least one of a heavy or light chain. Sources of such nucleic acid are well known to those skilled in the art and, for example, may be obtained from a hybridoma producing an antibody against a 21617 or 55562 polypeptide or fragment thereof. The recombinant DNA encoding the humanized antibody, or fragment thereof, can then be cloned into an appropriate expression vector.

Humanized or CDR-grafted antibodies can be produced by CDR-grafting or CDR substitution, wherein one, two, or all CDR's of an immunoglobulin chain can be replaced. See e.g., U.S. Pat. No. 5,225,539; Jones et al. 1986 Nature 321:552-525; Verhoeyan et al. 1988 Science 239:1534; Beidler et al. 1988 J. Immunol. 141:4053-4060; Winter U.S. Pat. No. 5,225,539, the contents of all of which are hereby expressly incorporated by reference. Winter describes a CDR-grafting method which may be used to prepare the humanized antibodies of the present invention (UK Patent Application GB 2188638A, filed on Mar. 26, 1987; Winter U.S. Pat. No. 5,225,539), the contents of which is expressly incorporated by reference.

Also within the scope of the invention are humanized antibodies in which specific amino acids have been substituted, deleted or added. Preferred humanized antibodies have amino acid substitutions in the framework region, such as to improve binding to the antigen. For example, a humanized antibody will have framework residues identical to the donor framework residue or to another amino acid other than the recipient framework residue. To generate such antibodies, a selected, small number of acceptor framework residues of the humanized immunoglobulin chain can be replaced by the corresponding donor amino acids. Preferred locations of the substitutions include amino acid residues adjacent to the CDR, or which are capable of interacting with a CDR (see e.g., U.S. Pat. No. 5,585,089). Criteria for selecting amino acids from the donor are described in U.S. Pat. No. 5,585,089, e.g., columns 12-16 of U.S. Pat. No. 5,585,089, the e.g., columns 12-16 of U.S. Pat. No. 5,585,089, the contents of which are hereby incorporated by reference. Other techniques for humanizing antibodies are described in Padlan et al. EP 519596 A1, published on Dec. 23, 1992.

In preferred embodiments an antibody can be made by immunizing with purified 21617 or 55562 antigen, or a fragment thereof, e.g., a fragment described herein, membrane associated antigen, tissue, e.g., crude tissue preparations, whole cells, preferably living cells, lysed cells, or cell fractions, e.g., membrane fractions or cytosolic fractions.

A full-length 21617 or 55562 protein or, antigenic peptide fragment of 21617 or 55562 can be used as an immunogen or can be used to identify anti-21617 or 55562 antibodies made with other immunogens, e.g., cells, membrane preparations, and the like. The antigenic peptide of 21617 or 55562 should include at least 8 amino acid residues of the amino acid sequence shown in SEQ ID NO:64 or SEQ ID NO:67 and encompasses an epitope of 21617 or 55562. Preferably, the antigenic peptide includes at least 10 amino acid residues, more preferably at least 15 amino acid residues, even more preferably at least 20 amino acid residues, and most preferably at least 30 amino acid residues.

Fragments of 21617 can be used, e.g., to characterize the specificity of an antibody or to make immunogens. For example, fragments of 21617 which include, e.g., residues about 68 to 77, 222 to 236, or 325 to 340 of SEQ ID NO:64 can be used to make antibodies against hydrophilic regions of the 21617 protein. Similarly, fragments of 21617 which include, e.g., residues 1 to 20, 191 to 203, or 293 to 310 of SEQ ID NO:64 can be used to make an antibody against a hydrophobic region of the 21617 protein; a fragment of 21617 which includes, e.g., residues about 37 to 249 of SEQ ID NO:64 can be used to make an antibody against the short chain dehydrogenase region of the 21617 protein; and a fragment of 21617 which includes, e.g., about amino acid residues 210 to 220 of SEQ ID NO:64 can be used to make an antibody against the dehydrogenase family signature motif of the 21617 protein.

Fragments of 55562 can be used, e.g., to characterize the specificity of an antibody or to make immunogens. For example, fragments of 55562 which include, e.g., residues about 2 to 9, 95 to 110, or 259 to 273 of SEQ ID NO:67 can be used to make antibodies against hydrophilic regions of the 55562 protein. Similarly, fragments of 55562 which include residues from about amino acid 39 to 44, 66 to 76, or 156 to 167 of SEQ ID NO:67 can be used to make an antibody against a hydrophobic region of the 55562 protein; a fragment of 55562 which include residues about 40 to 73 can be used to make an antibody against the TPR region of the 55562 protein; and fragments of 55562 which include about amino acid residues 40 to 266 can be used to make an antibody against the PD314595 homology domain of the 55562 protein.

Antibodies reactive with, or specific for, any of these regions, or other regions or domains described herein are provided.

Antibodies which bind only native 21617 or 55562 protein, only denatured or otherwise non-native 21617 or 55562 protein, or which bind both, are with in the invention. Antibodies with linear or conformational epitopes are within the invention. Conformational epitopes can sometimes be identified by identifying antibodies which bind to native but not denatured 21617 or 55562 protein.

Preferred epitopes encompassed by the antigenic peptide are regions of 21617 or 55562 are located on the surface of the protein, e.g., hydrophilic regions, as well as regions with high antigenicity. For example, an Emini surface probability analysis of the human 21617 or 55562 protein sequence can be used to indicate the regions that have a particularly high probability of being localized to the surface of the 21617 or 55562 protein and are thus likely to constitute surface residues useful for targeting antibody production.

In preferred embodiments the antibodies can bind one or more of purified antigen, membrane associated antigen, tissue, e.g., tissue sections, whole cells, preferably living cells, lysed cells, cell fractions, e.g., membrane fractions or cytosolic fractions.

The anti-21617 or 55562 antibody can be a single chain antibody. A single-chain antibody (scFV) may be engineered (see, for example, Colcher, D. et al. (1999) Ann NY Acad Sci 880:263-80; and Reiter, Y. (1996) Clin Cancer Res 2:245-52). The single chain antibody can be dimerized or multimerized to generate multivalent antibodies having specificities for different epitopes of the same target 21617 or 55562 protein.

In a preferred embodiment the antibody has effector function and/or can fix complement. In other embodiments the antibody does not recruit effector cells; or fix complement.

In a preferred embodiment, the antibody has reduced or no ability to bind an Fc receptor. For example, it is a isotype or subtype, fragment or other mutant, which does not support binding to an Fe receptor, e.g., it has a mutagenized or deleted Fe receptor binding region.

In a preferred embodiment, an anti-21617 antibody alters (e.g., increases or decreases) the dehydrogenase activity of a 21617 polypeptide. For example, the antibody can bind at or in proximity to the active site, e.g., to an epitope that includes a residue located from about amino acid 210 to 220 of SEQ ID NO:64.

In a preferred embodiment, an anti-21617 or 55562 antibody alters (e.g., increases or decreases) the activity of a 55562 polypeptide. For example, the antibody can bind at or in proximity to the tetratricopeptide repeat domain of 55562, e.g., to an epitope that includes a residue located from about 40 to 73 of SEQ ID NO:67.

The antibody can be coupled to a toxin, e.g., a polypeptide toxin, e,g, ricin or diphtheria toxin or active fragment hereof, or a radioactive nucleus, or imaging agent, e.g. a radioactive, enzymatic, or other, e.g., imaging agent, e.g., a NMR contrast agent. Labels that produce detectable radioactive emissions or fluorescence are preferred.

An anti-21617 or 55562 antibody (e.g., monoclonal antibody) can be used to isolate 21617 or 55562 by standard techniques, such as affinity chromatography or immunoprecipitation. Moreover, an anti-21617 or 55562 antibody can be used to detect 21617 or 55562 protein (e.g., in a cellular lysate or cell supernatant) in order to evaluate the abundance and pattern of expression of the protein. Anti-21617 or 55562 antibodies can be used diagnostically to monitor protein levels in tissue as part of a clinical testing procedure, e.g., to determine the efficacy of a given treatment regimen. Detection can be facilitated by coupling (i.e., physically linking) the antibody to a detectable substance (i.e., antibody labelling). Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

The invention also includes a nucleic acid which encodes an anti-21617 or 55562 antibody, e.g., an anti-21617 or 55562 antibody described herein. Also included are vectors which include the nucleic acid and cells transformed with the nucleic acid, particularly cells which are useful for producing an antibody, e.g., mammalian cells, e.g. CHO or lymphatic cells.

The invention also includes cell lines, e.g., hybridomas, which make an anti-21617 or 55562 antibody, e.g., an antibody described herein, and method of using said cells to make a 21617 or 55562 antibody.

21617 and 55562 Recombinant Expression Vectors, Host Cells and Genetically Engineered Cells

In another aspect, the invention includes, vectors, preferably expression vectors, containing a nucleic acid encoding a polypeptide described herein. As used herein, the term “vector” refers to a nucleic acid molecule capable of transporting another nucleic acid to which it has been linked and can include a plasmid, cosmid or viral vector. The vector can be capable of autonomous replication or it can integrate into a host DNA. Viral vectors include, e.g., replication defective retroviruses, adenoviruses and adeno-associated viruses.

A vector can include a 21617 or 55562 nucleic acid in a form suitable for expression of the nucleic acid in a host cell. Preferably the recombinant expression vector includes one or more regulatory sequences operatively linked to the nucleic acid sequence to be expressed. The term “regulatory sequence” includes promoters, enhancers and other expression control elements (e.g., polyadenylation signals). Regulatory sequences include those which direct constitutive expression of a nucleotide sequence, as well as tissue-specific regulatory and/or inducible sequences. The design of the expression vector can depend on such factors as the choice of the host cell to be transformed, the level of expression of protein desired, and the like. The expression vectors of the invention can be introduced into host cells to thereby produce proteins or polypeptides, including fusion proteins or polypeptides, encoded by nucleic acids as described herein (e.g., 21617 or 55562 proteins, mutant forms of 21617 or 55562 proteins, fusion proteins, and the like).

The recombinant expression vectors of the invention can be designed for expression of 21617 or 55562 proteins in prokaryotic or eukaryotic cells. For example, polypeptides of the invention can be expressed in E. coli, insect cells (e.g., using baculovirus expression vectors), yeast cells or mammalian cells. Suitable host cells are discussed further in Goeddel, (1990) Gene Expression Technology: Methods in Enzymology 185, Academic Press, San Diego, Calif. Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith, D. B. and Johnson, K. S. (1988) Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

Purified fusion proteins can be used in 21617 or 55562 activity assays, (e.g., direct assays or competitive assays described in detail below), or to generate antibodies specific for 21617 or 55562 proteins. In a preferred embodiment, a fusion protein expressed in a retroviral expression vector of the present invention can be used to infect bone marrow cells which are subsequently transplanted into irradiated recipients. The pathology of the subject recipient is then examined after sufficient time has passed (e.g., six weeks).

To maximize recombinant protein expression in E. coli is to express the protein in a host bacteria with an impaired capacity to proteolytically cleave the recombinant protein (Gottesman, S., (1990) Gene Expression Technology: Methods in Enzymology 185, Academic Press, San Diego, Calif. 119-128). Another strategy is to alter the nucleic acid sequence of the nucleic acid to be inserted into an expression vector so that the individual codons for each amino acid are those preferentially utilized in E. coli (Wada et al., (1992) Nucleic Acids Res. 20:2111-2118). Such alteration of nucleic acid sequences of the invention can be carried out by standard DNA synthesis techniques.

The 21617 or 55562 expression vector can be a yeast expression vector, a vector for expression in insect cells, e.g., a baculovirus expression vector or a vector suitable for expression in mammalian cells.

When used in mammalian cells, the expression vector's control functions can be provided by viral regulatory elements. For example, commonly used promoters are derived from polyoma, Adenovirus 2, cytomegalovirus and Simian Virus 40.

In another embodiment, the promoter is an inducible promoter, e.g., a promoter regulated by a steroid hormone, by a polypeptide hormone (e.g., by means of a signal transduction pathway), or by a heterologous polypeptide (e.g., the tetracycline-inducible systems, “Tet-On” and “Tet-Off”; see, e.g., Clontech Inc., CA, Gossen and Bujard (1992) Proc. Natl. Acad. Sci. USA 89:5547, and Paillard (1989) Human Gene Therapy 9:983).

In another embodiment, the recombinant mammalian expression vector is capable of directing expression of the nucleic acid preferentially in a particular cell type (e.g., tissue-specific regulatory elements are used to express the nucleic acid). Non-limiting examples of suitable tissue-specific promoters include the albumin promoter (liver-specific; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) Proc. Natl. Acad. Sci. USA 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, for example, the murine hox promoters (Kessel and Gruss (1990) Science 249:374-379) and the (x-fetoprotein promoter (Campes and Tilghman (1989) Genes Dev. 3:537-546).

The invention further provides a recombinant expression vector comprising a DNA molecule of the invention cloned into the expression vector in an antisense orientation. Regulatory sequences (e.g., viral promoters and/or enhancers) operatively linked to a nucleic acid cloned in the antisense orientation can be chosen which direct the constitutive, tissue specific or cell type specific expression of antisense RNA in a variety of cell types. The antisense expression vector can be in the form of a recombinant plasmid, phagemid or attenuated virus.

Another aspect the invention provides a host cell which includes a nucleic acid molecule described herein, e.g., a 21617 or 55562 nucleic acid molecule within a recombinant expression vector or a 21617 or 55562 nucleic acid molecule containing sequences which allow it to homologously recombine into a specific site of the host cell's genome. The terms “host cell” and “recombinant host cell” are used interchangeably herein. Such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, a 21617 or 55562 protein can be expressed in bacterial cells (such as E. coli), insect cells, yeast or mammalian cells (such as Chinese hamster ovary cells (CHO) or COS cells (African green monkey kidney cells CV-1 origin SV40 cells; Gluzman (1981) CellI23:175-182)). Other suitable host cells are known to those skilled in the art.

Vector DNA can be introduced into host cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation.

A host cell of the invention can be used to produce (i.e., express) a 21617 or 55562 protein. Accordingly, the invention further provides methods for producing a 21617 or 55562 protein using the host cells of the invention. In one embodiment, the method includes culturing the host cell of the invention (into which a recombinant expression vector encoding a 21617 or 55562 protein has been introduced) in a suitable medium such that a 21617 or 55562 protein is produced. In another embodiment, the method further includes isolating a 21617 or 55562 protein from the medium or the host cell.

In another aspect, the invention features, a cell or purified preparation of cells which include a 21617 or 55562 transgene, or which otherwise misexpress 21617 or 55562. The cell preparation can consist of human or non-human cells, e.g., rodent cells, e.g., mouse or rat cells, rabbit cells, or pig cells. In preferred embodiments, the cell or cells include a 21617 or 55562 transgene, e.g., a heterologous form of a 21617 or 55562, e.g., a gene derived from humans (in the case of a non-human cell). The 21617 or 55562 transgene can be misexpressed, e.g., overexpressed or underexpressed. In other preferred embodiments, the cell or cells include a gene that mis-expresses an endogenous 21617 or 55562, e.g., a gene the expression of which is disrupted, e.g., a knockout. Such cells can serve as a model for studying disorders that are related to mutated or mis-expressed 21617 or 55562 alleles or for use in drug screening.

In another aspect, the invention features, a human cell, e.g., a hematopoietic, hepatic, neural, or muscle stem cell, transformed with nucleic acid which encodes a subject 21617 or 55562 polypeptide.

Also provided are cells, preferably human cells, e.g., human hematopoietic, hepatic, neural, or muscle cells, fibroblast cells, or tumor-derived cells in which an endogenous 21617 or 55562 is under the control of a regulatory sequence that does not normally control the expression of the endogenous 21617 or 55562 gene. The expression characteristics of an endogenous gene within a cell, e.g., a cell line or microorganism, can be modified by inserting a heterologous DNA regulatory element into the genome of the cell such that the inserted regulatory element is operably linked to the endogenous 21617 or 55562 gene. For example, an endogenous 21617 or 55562 gene which is “transcriptionally silent,” e.g., not normally expressed, or expressed only at very low levels, may be activated by inserting a regulatory element which is capable of promoting the expression of a normally expressed gene product in that cell. Techniques such as targeted homologous recombinations, can be used to insert the heterologous DNA as described in, e.g., Chappel, U.S. Pat. No. 5,272,071; WO 91/06667, published in May 16, 1991.

In a preferred embodiment, recombinant cells described herein can be used for replacement therapy in a subject. For example, a nucleic acid encoding a 21617 or 55562 polypeptide operably linked to an inducible promoter (e.g., a steroid hormone receptor-regulated promoter) is introduced into a human or nonhuman, e.g., mammalian, e.g., porcine recombinant cell. The cell is cultivated and encapsulated in a biocompatible material, such as poly-lysine alginate, and subsequently implanted into the subject. See, e.g., Lanza (1996) Nat. Biotechnol. 14:1107; Joki et al. (2001) Nat. Biotechnol. 19:35; and U.S. Pat. No. 5,876,742. Production of 21617 or 55562 polypeptide can be regulated in the subject by administering an agent (e.g., a steroid hormone) to the subject. In another preferred embodiment, the implanted recombinant cells express and secrete an antibody specific for a 21617 or 55562 polypeptide. The antibody can be any antibody or any antibody derivative described herein.

21617 and 55562 Transgenic Animals

The invention provides non-human transgenic animals. Such animals are useful for studying the function and/or activity of a 21617 or 55562 protein and for identifying and/or evaluating modulators of 21617 or 55562 activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, and the like. A transgene is exogenous DNA or a rearrangement, e.g., a deletion of endogenous chromosomal DNA, which preferably is integrated into or occurs in the genome of the cells of a transgenic animal. A transgene can direct the expression of an encoded gene product in one or more cell types or tissues of the transgenic animal, other transgenes, e.g., a knockout, reduce expression. Thus, a transgenic animal can be one in which an endogenous 21617 or 55562 gene has been altered by, e.g., by homologous recombination between the endogenous gene and an exogenous DNA molecule introduced into a cell of the animal, e.g., an embryonic cell of the animal, prior to development of the animal.

Intronic sequences and polyadenylation signals can also be included in the transgene to increase the efficiency of expression of the transgene. A tissue-specific regulatory sequence(s) can be operably linked to a transgene of the invention to direct expression of a 21617 or 55562 protein to particular cells. A transgenic founder animal can be identified based upon the presence of a 21617 or 55562 transgene in its genome and/or expression of 21617 or 55562 mRNA in tissues or cells of the animals. A transgenic founder animal can then be used to breed additional animals carrying the transgene. Moreover, transgenic animals carrying a transgene encoding a 21617 or 55562 protein can further be bred to other transgenic animals carrying other transgenes.

21617 or 55562 proteins or polypeptides can be expressed in transgenic animals or plants, e.g., a nucleic acid encoding the protein or polypeptide can be introduced into the genome of an animal. In preferred embodiments the nucleic acid is placed under the control of a tissue specific promoter, e.g., a milk or egg specific promoter, and recovered from the milk or eggs produced by the animal. Suitable animals are mice, pigs, cows, goats, and sheep.

The invention also includes a population of cells from a transgenic animal, as discussed, e.g., below.

Uses of 21617 and 55562

The nucleic acid molecules, proteins, protein homologues, and antibodies described herein can be used in one or more of the following methods: a) screening assays; b) predictive medicine (e.g., diagnostic assays, prognostic assays, monitoring clinical trials, and pharmacogenetics); and c) methods of treatment (e.g., therapeutic and prophylactic).

In addition, 21617 proteins of the invention can be used in vitro, e.g., for the oxidation or reduction of substrate molecules in a synthetic or diagnostic process. Dehydrogenase and/or reductase activity is widely used for the quantitation of ethanol in biological fluids. It can also be used in coupled enzyme reactions for determination of metabolites in biological fluids. Dehydrogenases catalyzes the oxidation of alcohol and the reduction of aldehydes as shown below:

Acetaldehyde+NADH+H+<->Ethanol+NAD+

As carried out by yeasts, this fermentation generates the alcohol in alcoholic beverages. Yeasts used in baking also carry out the alcoholic fermentation; the CO2 produced by pyruvate decarboxylation causes bread to rise, and the ethanol produced evaporates during baking.

The isolated nucleic acid molecules of the invention can be used, for example, to express a 21617 or 55562 protein (e.g., via a recombinant expression vector in a host cell in gene therapy applications), to detect a 21617 or 55562 mRNA (e.g., in a biological sample) or a genetic alteration in a 21617 or 55562 gene, and to modulate 21617 or 55562 activity, as described further below. The 21617 or 55562 proteins can be used to treat disorders characterized by insufficient or excessive production of a 21617 or 55562 substrate or production of 21617 or 55562 inhibitors. In addition, the 21617 or 55562 proteins can be used to screen for naturally occurring 21617 or 55562 substrates, to screen for drugs or compounds which modulate 21617 or 55562 activity, as well as to treat disorders characterized by insufficient or excessive production of 21617 or 55562 protein or production of 21617 or 55562 protein forms which have decreased, aberrant or unwanted activity compared to 21617 or 55562 wild type protein (e.g., cellular proliferation and/or differentiation disorders, metabolic disorders, kidney disorders, endothelial cell disorders, neural disorders, and viral disorders). Moreover, the anti-21617 or 55562 antibodies of the invention can be used to detect and isolate 21617 or 55562 proteins, regulate the bioavailability of 21617 or 55562 proteins, and modulate 21617 or 55562 activity.

A method of evaluating a compound for the ability to interact with, e.g., bind, a subject 21617 or 55562 polypeptide is provided. The method includes: contacting the compound with the subject 21617 or 55562 polypeptide; and evaluating ability of the compound to interact with, e.g., to bind or form a complex with the subject 21617 or 55562 polypeptide. This method can be performed in vitro, e.g., in a cell free system, or in vivo, e.g., in a two-hybrid interaction trap assay. This method can be used to identify naturally occurring molecules that interact with subject 21617 or 55562 polypeptide. It can also be used to find natural or synthetic inhibitors of subject 21617 or 55562 polypeptide. Screening methods are discussed in more detail below.

21617 and 55562 Screening Assays

The invention provides methods (also referred to herein as “screening assays”) for identifying modulators, i.e., candidate or test compounds or agents (e.g., proteins, peptides, peptidomimetics, peptoids, small molecules or other drugs) which bind to 21617 or 55562 proteins, have a stimulatory or inhibitory effect on, for example, 21617 or 55562 expression or 21617 or 55562 activity, or have a stimulatory or inhibitory effect on, for example, the expression or activity of a 21617 or 55562 substrate. Compounds thus identified can be used to modulate the activity of target gene products (e.g., 21617 or 55562 genes) in a therapeutic protocol, to elaborate the biological function of the target gene product, or to identify compounds that disrupt normal target gene interactions.

In one embodiment, the invention provides assays for screening candidate or test compounds which are substrates of a 21617 or 55562 protein or polypeptide or a biologically active portion thereof. In another embodiment, the invention provides assays for screening candidate or test compounds that bind to or modulate an activity of a 21617 or 55562 protein or polypeptide or a biologically active portion thereof.

In one embodiment, an activity of a 21617 protein can be assayed as follows: (a) contact a test substrate molecule (e.g., a steroid or alcohol) with a 21617 protein or functional fragment thereof in the presence of a coenzyme, e.g., NAD; and (b) evaluate the ability the 21617 protein or functional fragment thereof to catalyze a reaction, e.g., oxidize an alcohol group or reduce a carbonyl group present on the test substrate. In an exemplary embodiment, the ability of a 21617 molecule to catalyze a reaction is evaluated by evaluating the presence or absence of the substrate or the enzymatic product of the reaction at the end of the assay, e.g., by evaluating the slope of absorbance of the substrate or end product through the assay period.

The test compounds of the present invention can be obtained using any of the numerous approaches in combinatorial library methods known in the art, including: biological libraries; peptoid libraries (libraries of molecules having the functionalities of peptides, but with a novel, non-peptide backbone which are resistant to enzymatic degradation but which nevertheless remain bioactive; see, e.g., Zuckermann, R. N. et al. (1994) J. Med. Chem. 37:2678-85); spatially addressable parallel solid phase or solution phase libraries; synthetic library methods requiring deconvolution; the ‘one-bead one-compound’ library method; and synthetic library methods using affinity chromatography selection. The biological library and peptoid library approaches are limited to peptide libraries, while the other four approaches are applicable to peptide, non-peptide oligomer or small molecule libraries of compounds (Lam (1997) Anticancer Drug Des. 12:145).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and Gallop et al. (1994) J. Med. Chem. 37:1233.

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner, U.S. Pat. No. 5,223,409), spores (Ladner U.S. Pat. No. 5,223,409), plasmids (Cull et al. (1992) Proc Natl Acad Sci USA 89:1865-1869) or on phage (Scott and Smith (1990) Science 249:386-390; Devlin (1990) Science 249:404-406; Cwirla et al. (1990) Proc. Natl. Acad. Sci. 87:6378-6382; Felici (1991) J. Mol. Biol. 222:301-310; Ladner supra.).

In one embodiment, an assay is a cell-based assay in which a cell which expresses a 21617 or 55562 protein or biologically active portion thereof is contacted with a test compound, and the ability of the test compound to modulate 21617 or 55562 activity is determined. Determining the ability of the test compound to modulate 21617 or 55562 activity can be accomplished by monitoring, for example, cellular proliferation and/or differentiation. The cell, for example, can be of mammalian origin, e.g., human.

The ability of the test compound to modulate 21617 or 55562 binding to a compound, e.g., a 21617 or 55562 substrate, or to bind to 21617 or 55562 can also be evaluated. This can be accomplished, for example, by coupling the compound, e.g., the substrate, with a radioisotope or enzymatic label such that binding of the compound, e.g., the substrate, to 21617 or 55562 can be determined by detecting the labeled compound, e.g., substrate, in a complex. Alternatively, 21617 or 55562 could be coupled with a radioisotope or enzymatic label to monitor the ability of a test compound to modulate 21617 or 55562 binding to a 21617 or 55562 substrate in a complex. For example, compounds (e.g., 21617 or 55562 substrates) can be labeled with ¹²⁵I, ³⁵S, ¹⁴C, or ³H, either directly or indirectly, and the radioisotope detected by direct counting of radioemmission or by scintillation counting. Alternatively, compounds can be enzymatically labeled with, for example, horseradish peroxidase, alkaline phosphatase, or luciferase, and the enzymatic label detected by determination of conversion of an appropriate substrate to product.

The ability of a compound (e.g., a 21617 or 55562 substrate) to interact with 21617 or 55562 with or without the labeling of any of the interactants can be evaluated. For example, a microphysiometer can be used to detect the interaction of a compound with 21617 or 55562 without the labeling of either the compound or the 21617 or 55562. McConnell, H. M. et al. (1992) Science 257:1906-1912. As used herein, a “microphysiometer” (e.g., Cytosensor) is an analytical instrument that measures the rate at which a cell acidifies its environment using a light-addressable potentiometric sensor (LAPS). Changes in this acidification rate can be used as an indicator of the interaction between a compound and 21617 or 55562.

In yet another embodiment, a cell-free assay is provided in which a 21617 or 55562 protein or biologically active portion thereof is contacted with a test compound and the ability of the test compound to bind to the 21617 or 55562 protein or biologically active portion thereof is evaluated. Preferred biologically active portions of the 21617 or 55562 proteins to be used in assays of the present invention include fragments which participate in interactions with non-21617 or 55562 molecules, e.g., fragments with high surface probability scores.

Soluble and/or membrane-bound forms of isolated proteins (e.g., 21617 or 55562 proteins or biologically active portions thereof) can be used in the cell-free assays of the invention. When membrane-bound forms of the protein are used, it may be desirable to utilize a solubilizing agent. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)ₙ, 3-[(3-cholamidopropyl)dimethylamminio]-1-propane sulfonate (CHAPS), 3-[(3-cholamidopropyl)dimethylamminio]-2-hydroxy-1-propane sulfonate (CHAPSO), or N-dodecyl=N,N-dimethyl-3-ammonio-1-propane sulfonate.

Cell-free assays involve preparing a reaction mixture of the target gene protein and the test compound under conditions and for a time sufficient to allow the two components to interact and bind, thus forming a complex that can be removed and/or detected.

The interaction between two molecules can also be detected, e.g., using fluorescence energy transfer (FET) (see, for example, Lakowicz et al., U.S. Pat. No. 5,631,169; Stavrianopoulos, et al., U.S. Pat. No. 4,868,103). A fluorophore label on the first, ‘donor’ molecule is selected such that its emitted fluorescent energy will be absorbed by a fluorescent label on a second, ‘acceptor’ molecule, which in turn is able to fluoresce due to the absorbed energy. Alternately, the ‘donor’ protein molecule may simply utilize the natural fluorescent energy of tryptophan residues. Labels are chosen that emit different wavelengths of light, such that the ‘acceptor’ molecule label may be differentiated from that of the ‘donor’. Since the efficiency of energy transfer between the labels is related to the distance separating the molecules, the spatial relationship between the molecules can be assessed. In a situation in which binding occurs between the molecules, the fluorescent emission of the ‘acceptor’ molecule label in the assay should be maximal. An FET binding event can be conveniently measured through standard fluorometric detection means well known in the art (e.g., using a fluorimeter).

In another embodiment, determining the ability of the 21617 or 55562 protein to bind to a target molecule can be accomplished using real-time Biomolecular Interaction Analysis (BIA) (see, e.g., Sjolander, S. and Urbaniczky, C. (1991) Anal. Chem. 63:2338-2345 and Szabo et al. (1995) Curr. Opin. Struct. Biol. 5:699-705). “Surface plasmon resonance” or “BIA” detects biospecific interactions in real time, without labeling any of the interactants (e.g., BIAcore). Changes in the mass at the binding surface (indicative of a binding event) result in alterations of the refractive index of light near the surface (the optical phenomenon of surface plasmon resonance (SPR)), resulting in a detectable signal which can be used as an indication of real-time reactions between biological molecules.

In one embodiment, the target gene product or the test substance is anchored onto a solid phase. The target gene product/test compound complexes anchored on the solid phase can be detected at the end of the reaction. Preferably, the target gene product can be anchored onto a solid surface, and the test compound, (which is not anchored), can be labeled, either directly or indirectly, with detectable labels discussed herein.

It may be desirable to immobilize either 21617 or 55562, an anti-21617 or 55562 antibody or its target molecule to facilitate separation of complexed from uncomplexed forms of one or both of the proteins, as well as to accommodate automation of the assay. Binding of a test compound to a 21617 or 55562 protein, or interaction of a 21617 or 55562 protein with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided which adds a domain that allows one or both of the proteins to be bound to a matrix.

For example, glutathione-S-transferase/21617 or 55562 fusion proteins or glutathione-S-transferase/target fusion proteins can be adsorbed onto glutathione sepharose beads (Sigma Chemical, St. Louis, Mo.) or glutathione derivatized microtiter plates, which are then combined with the test compound or the test compound and either the non-adsorbed target protein or 21617 or 55562 protein, and the mixture incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described above. Alternatively, the complexes can be dissociated from the matrix, and the level of 21617 or 55562 binding or activity determined using standard techniques.

Other techniques for immobilizing either a 21617 or 55562 protein or a target molecule on matrices include using conjugation of biotin and streptavidin. Biotinylated 21617 or 55562 protein or target molecules can be prepared from biotin-NHS(N-hydroxy-succinimide) using techniques known in the art (e.g., biotinylation kit, Pierce Chemicals, Rockford, Ill.), and immobilized in the wells of streptavidin-coated 96 well plates (Pierce Chemical).

In order to conduct the assay, the non-immobilized component is added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways. Where the previously non-immobilized component is pre-labeled, the detection of label immobilized on the surface indicates that complexes were formed. Where the previously non-immobilized component is not pre-labeled, an indirect label can be used to detect complexes anchored on the surface; e.g., using a labeled antibody specific for the immobilized component (the antibody, in turn, can be directly labeled or indirectly labeled with, e.g., a labeled anti-Ig antibody).

In one embodiment, this assay is performed utilizing antibodies reactive with 21617 or 55562 protein or target molecules but which do not interfere with binding of the 21617 or 55562 protein to its target molecule. Such antibodies can be derivatized to the wells of the plate, and unbound target or 21617 or 55562 protein trapped in the wells by antibody conjugation. Methods for detecting such complexes, in addition to those described above for the GST-immobilized complexes, include immunodetection of complexes using antibodies reactive with the 21617 or 55562 protein or target molecule, as well as enzyme-linked assays which rely on detecting an enzymatic activity associated with the 21617 or 55562 protein or target molecule.

Alternatively, cell free assays can be conducted in a liquid phase. In such an assay, the reaction products are separated from unreacted components, by any of a number of standard techniques, including but not limited to: differential centrifugation (see, for example, Rivas, G., and Minton, A. P., (1993) Trends Biochem Sci 18:284-7); chromatography (gel filtration chromatography, ion-exchange chromatography); electrophoresis (see, e.g., Ausubel, F. et al., eds. Current Protocols in Molecular Biology 1999, J. Wiley: New York.); and immunoprecipitation (see, for example, Ausubel, F. et al., eds. (1999) Current Protocols in Molecular Biology, J. Wiley: New York). Such resins and chromatographic techniques are known to one skilled in the art (see, e.g., Heegaard, N. H., (1998) J Mol Recognit 11:141-8; Hage, D. S., and Tweed, S. A. (1997) J Chromatogr B Biomed Sci Appl. 699:499-525). Further, fluorescence energy transfer may also be conveniently utilized, as described herein, to detect binding without further purification of the complex from solution.

In a preferred embodiment, the assay includes contacting the 21617 or 55562 protein or biologically active portion thereof with a known compound which binds 21617 or 55562 to form an assay mixture, contacting the assay mixture with a test compound, and determining the ability of the test compound to interact with a 21617 or 55562 protein, wherein determining the ability of the test compound to interact with a 21617 or 55562 protein includes determining the ability of the test compound to preferentially bind to 21617 or 55562 or biologically active portion thereof, or to modulate the activity of a target molecule, as compared to the known compound.

The target gene products of the invention can, in vivo, interact with one or more cellular or extracellular macromolecules, such as proteins. For the purposes of this discussion, such cellular and extracellular macromolecules are referred to herein as “binding partners.”

Compounds that disrupt such interactions can be useful in regulating the activity of the target gene product. Such compounds can include, but are not limited to molecules such as antibodies, peptides, and small molecules. The preferred target genes/products for use in this embodiment are the 21617 or 55562 genes herein identified. In an alternative embodiment, the invention provides methods for determining the ability of the test compound to modulate the activity of a 21617 or 55562 protein through modulation of the activity of a downstream effector of a 21617 or 55562 target molecule. For example, the activity of the effector molecule on an appropriate target can be determined, or the binding of the effector to an appropriate target can be determined, as previously described.

To identify compounds that interfere with the interaction between the target gene product and its cellular or extracellular binding partner(s), a reaction mixture containing the target gene product and the binding partner is prepared, under conditions and for a time sufficient, to allow the two products to form complex. In order to test an inhibitory agent, the reaction mixture is provided in the presence and absence of the test compound. The test compound can be initially included in the reaction mixture, or can be added at a time subsequent to the addition of the target gene and its cellular or extracellular binding partner. Control reaction mixtures are incubated without the test compound or with a placebo. The formation of any complexes between the target gene product and the cellular or extracellular binding partner is then detected. The formation of a complex in the control reaction, but not in the reaction mixture containing the test compound, indicates that the compound interferes with the interaction of the target gene product and the interactive binding partner. Additionally, complex formation within reaction mixtures containing the test compound and normal target gene product can also be compared to complex formation within reaction mixtures containing the test compound and mutant target gene product. This comparison can be important in those cases wherein it is desirable to identify compounds that disrupt interactions of mutant but not normal target gene products.

These assays can be conducted in a heterogeneous or homogeneous format. Heterogeneous assays involve anchoring either the target gene product or the binding partner onto a solid phase, and detecting complexes anchored on the solid phase at the end of the reaction. In homogeneous assays, the entire reaction is carried out in a liquid phase. In either approach, the order of addition of reactants can be varied to obtain different information about the compounds being tested. For example, test compounds that interfere with the interaction between the target gene products and the binding partners, e.g., by competition, can be identified by conducting the reaction in the presence of the test substance. Alternatively, test compounds that disrupt preformed complexes, e.g., compounds with higher binding constants that displace one of the components from the complex, can be tested by adding the test compound to the reaction mixture after complexes have been formed. The various formats are briefly described below.

In a heterogeneous assay system, either the target gene product or the interactive cellular or extracellular binding partner, is anchored onto a solid surface (e.g., a microtiter plate), while the non-anchored species is labeled, either directly or indirectly. The anchored species can be immobilized by non-covalent or covalent attachments. Alternatively, an immobilized antibody specific for the species to be anchored can be used to anchor the species to the solid surface.

In order to conduct the assay, the partner of the immobilized species is exposed to the coated surface with or without the test compound. After the reaction is complete, unreacted components are removed (e.g., by washing) and any complexes formed will remain immobilized on the solid surface. Where the non-immobilized species is pre-labeled, the detection of label immobilized on the surface indicates that complexes were formed. Where the non-immobilized species is not pre-labeled, an indirect label can be used to detect complexes anchored on the surface; e.g., using a labeled antibody specific for the initially non-immobilized species (the antibody, in turn, can be directly labeled or indirectly labeled with, e.g., a labeled anti-Ig antibody). Depending upon the order of addition of reaction components, test compounds that inhibit complex formation or that disrupt preformed complexes can be detected.

Alternatively, the reaction can be conducted in a liquid phase in the presence or absence of the test compound, the reaction products separated from unreacted components, and complexes detected; e.g., using an immobilized antibody specific for one of the binding components to anchor any complexes formed in solution, and a labeled antibody specific for the other partner to detect anchored complexes. Again, depending upon the order of addition of reactants to the liquid phase, test compounds that inhibit complex or that disrupt preformed complexes can be identified.

In an alternate embodiment of the invention, a homogeneous assay can be used. For example, a preformed complex of the target gene product and the interactive cellular or extracellular binding partner product is prepared in that either the target gene products or their binding partners are labeled, but the signal generated by the label is quenched due to complex formation (see, e.g., U.S. Pat. No. 4,109,496 that utilizes this approach for immunoassays). The addition of a test substance that competes with and displaces one of the species from the preformed complex will result in the generation of a signal above background. In this way, test substances that disrupt target gene product-binding partner interaction can be identified.

In yet another aspect, the 21617 or 55562 proteins can be used as “bait proteins” in a two-hybrid assay or three-hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Biotechniques 14:920-924; Iwabuchi et al. (1993) Oncogene 8:1693-1696; and Brent WO94/10300), to identify other proteins, which bind to or interact with 21617 or 55562 (“21617 or 55562-binding proteins” or “21617 or 55562-bp”) and are involved in 21617 or 55562 activity. Such 21617 or 55562-bps can be activators or inhibitors of signals by the 21617 or 55562 proteins or 21617 or 55562 targets as, for example, downstream elements of a 21617 or 55562-mediated signaling pathway.

The two-hybrid system is based on the modular nature of most transcription factors, which consist of separable DNA-binding and activation domains. Briefly, the assay utilizes two different DNA constructs. In one construct, the gene that codes for a 21617 or 55562 protein is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. (Alternatively the: 21617 or 55562 protein can be the fused to the activator domain.) If the “bait” and the “prey” proteins are able to interact, in vivo, forming a 21617 or 55562-dependent complex, the DNA-binding and activation domains of the transcription factor are brought into close proximity. This proximity allows transcription of a reporter gene (e.g., lacZ) which is operably linked to a transcriptional regulatory site responsive to the transcription factor. Expression of the reporter gene can be detected and cell colonies containing the functional transcription factor can be isolated and used to obtain the cloned gene which encodes the protein which interacts with the 21617 or 55562 protein.

In another embodiment, modulators of 21617 or 55562 expression are identified. For example, a cell or cell free mixture is contacted with a candidate compound and the expression of 21617 or 55562 mRNA or protein evaluated relative to the level of expression of 21617 or 55562 mRNA or protein in the absence of the candidate compound. When expression of 21617 or 55562 mRNA or protein is greater in the presence of the candidate compound than in its absence, the candidate compound is identified as a stimulator of 21617 or 55562 mRNA or protein expression. Alternatively, when expression of 21617 or 55562 mRNA or protein is less (statistically significantly less) in the presence of the candidate compound than in its absence, the candidate compound is identified as an inhibitor of 21617 or 55562 mRNA or protein expression. The level of 21617 or 55562 mRNA or protein expression can be determined by methods described herein for detecting 21617 or 55562 mRNA or protein.

In another aspect, the invention pertains to a combination of two or more of the assays described herein. For example, a modulating agent can be identified using a cell-based or a cell free assay, and the ability of the agent to modulate the activity of a 21617 or 55562 protein can be confirmed in vivo, e.g., in an animal such as an animal model for a cellular proliferation and/or differentiation disorder, metabolic disorder, kidney disorder, endothelial cell disorder, neural disorder, or viral disorder.

This invention further pertains to novel agents identified by the above-described screening assays. Accordingly, it is within the scope of this invention to further use an agent identified as described herein (e.g., a 21617 or 55562 modulating agent, an antisense 21617 or 55562 nucleic acid molecule, a 21617 or 55562-specific antibody, or a 21617 or 55562-binding partner) in an appropriate animal model to determine the efficacy, toxicity, side effects, or mechanism of action, of treatment with such an agent. Furthermore, novel agents identified by the above-described screening assays can be used for treatments as described herein.

21617 and 55562 Detection Assays

Portions or fragments of the nucleic acid sequences identified herein can be used as polynucleotide reagents. For example, these sequences can be used to: (i) map their respective genes on a chromosome e.g., to locate gene regions associated with genetic disease or to associate 21617 or 55562 with a disease; (ii) identify an individual from a minute biological sample (tissue typing); and (iii) aid in forensic identification of a biological sample. These applications are described in the subsections below.

21617 and 55562 Chromosome Mapping

The 21617 or 55562 nucleotide sequences or portions thereof can be used to map the location of the 21617 or 55562 genes on a chromosome. This process is called chromosome mapping. Chromosome mapping is useful in correlating the 21617 or 55562 sequences with genes associated with disease.

Briefly, 21617 or 55562 genes can be mapped to chromosomes by preparing PCR primers (preferably 15-25 bp in length) from the 21617 or 55562 nucleotide sequences. These primers can then be used for PCR screening of somatic cell hybrids containing individual human chromosomes. Only those hybrids containing the human gene corresponding to the 21617 or 55562 sequences will yield an amplified fragment.

A panel of somatic cell hybrids in which each cell line contains either a single human chromosome or a small number of human chromosomes, and a full set of mouse chromosomes, can allow easy mapping of individual genes to specific human chromosomes. (D'Eustachio P. et al. (1983) Science 220:919-924).

Other mapping strategies e.g., in situ hybridization (described in Fan, Y. et al. (1990) Proc. Natl. Acad. Sci. USA, 87:6223-27), pre-screening with labeled flow-sorted chromosomes, and pre-selection by hybridization to chromosome specific cDNA libraries can be used to map 21617 or 55562 to a chromosomal location.

Fluorescence in situ hybridization (FISH) of a DNA sequence to a metaphase chromosomal spread can further be used to provide a precise chromosomal location in one step. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases will suffice to get good results at a reasonable amount of time. For a review of this technique, see Verma et al., Human Chromosomes: A Manual of Basic Techniques ((1988) Pergamon Press, New York).

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. (Such data are found, for example, in V. McKusick, Mendelian Inheritance in Man, available on-line through Johns Hopkins University Welch Medical Library). The relationship between a gene and a disease, mapped to the same chromosomal region, can then be identified through linkage analysis (co-inheritance of physically adjacent genes), described in, for example, Egeland, J. et al. (1987) Nature, 325:783-787.

Moreover, differences in the DNA sequences between individuals affected and unaffected with a disease associated with the 21617 or 55562 gene, can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

21617 and 55562 Tissue Typing

21617 or 55562 sequences can be used to identify individuals from biological samples using, e.g., restriction fragment length polymorphism (RFLP). In this technique, an individual's genomic DNA is digested with one or more restriction enzymes, the fragments separated, e.g., in a Southern blot, and probed to yield bands for identification. The sequences of the present invention are useful as additional DNA markers for RFLP (described in U.S. Pat. No. 5,272,057).

Furthermore, the sequences of the present invention can also be used to determine the actual base-by-base DNA sequence of selected portions of an individual's genome. Thus, the 21617 or 55562 nucleotide sequences described herein can be used to prepare two PCR primers from the 5′ and 3′ ends of the sequences. These primers can then be used to amplify an individual's DNA and subsequently sequence it. Panels of corresponding DNA sequences from individuals, prepared in this manner, can provide unique individual identifications, as each individual will have a unique set of such DNA sequences due to allelic differences.

Allelic variation occurs to some degree in the coding regions of these sequences, and to a greater degree in the noncoding regions. Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences of SEQ ID NO:63 OR SEQ ID NO:66 can provide positive individual identification with a panel of perhaps 10 to 1,000 primers which each yield a noncoding amplified sequence of 100 bases. If predicted coding sequences, such as those in SEQ ID NO:65 OR SEQ ID NO:68 are used, a more appropriate number of primers for positive individual identification would be 500-2,000.

If a panel of reagents from 21617 or 55562 nucleotide sequences described herein is used to generate a unique identification database for an individual, those same reagents can later be used to identify tissue from that individual. Using the unique identification database, positive identification of the individual, living or dead, can be made from extremely small tissue samples.

Use of Partial 21617 or 55562 Sequences in Forensic Biology

DNA-based identification techniques can also be used in forensic biology. To make such an identification, PCR technology can be used to amplify DNA sequences taken from very small biological samples such as tissues, e.g., hair or skin, or body fluids, e.g., blood, saliva, or semen found at a crime scene. The amplified sequence can then be compared to a standard, thereby allowing identification of the origin of the biological sample.

The sequences of the present invention can be used to provide polynucleotide reagents, e.g., PCR primers, targeted to specific loci in the human genome, which can enhance the reliability of DNA-based forensic identifications by, for example, providing another “identification marker” (i.e. another DNA sequence that is unique to a particular individual). As mentioned above, actual base sequence information can be used for identification as an accurate alternative to patterns formed by restriction enzyme generated fragments. Sequences targeted to noncoding regions of SEQ ID NO:63 or SEQ ID NO:66 (e.g., fragments derived from the noncoding regions of SEQ ID NO:63 or SEQ ID NO:66 having a length of at least 20 bases, preferably at least 30 bases) are particularly appropriate for this use.

The 21617 or 55562 nucleotide sequences described herein can further be used to provide polynucleotide reagents, e.g., labeled or labelable probes which can be used in, for example, an in situ hybridization technique, to identify a specific tissue. This can be very useful in cases where a forensic pathologist is presented with a tissue of unknown origin. Panels of such 21617 or 55562 probes can be used to identify tissue by species and/or by organ type.

In a similar fashion, these reagents, e.g., 21617 or 55562 primers or probes can be used to screen tissue culture for contamination (i.e. screen for the presence of a mixture of different types of cells in a culture).

Predictive Medicine of 21617 and 55562

The present invention also pertains to the field of predictive medicine in which diagnostic assays, prognostic assays, and monitoring clinical trials are used for prognostic (predictive) purposes to thereby treat an individual.

Generally, the invention provides, a method of determining if a subject is at risk for a disorder related to a lesion in or the misexpression of a gene which encodes 21617 or 55562.

Such disorders include, e.g., a disorder associated with the misexpression of 21617 or 55562 gene; a cellular proliferation and/or differentiation disorder, e.g., cancer, e.g., colon, colorectal, breast, lung, cervical, ovarian or liver cancer.

The method includes one or more of the following:

detecting, in a tissue of the subject, the presence or absence of a mutation which affects the expression of the 21617 or 55562 gene, or detecting the presence or absence of a mutation in a region which controls the expression of the gene, e.g., a mutation in the 5′ control region;

detecting, in a tissue of the subject, the presence or absence of a mutation which alters the structure of the 21617 or 55562 gene;

detecting, in a tissue of the subject, the misexpression of the 21617 or 55562 gene, at the mRNA level, e.g., detecting a non-wild type level of a mRNA;

detecting, in a tissue of the subject, the misexpression of the gene, at the protein level, e.g., detecting a non-wild type level of a 21617 or 55562 polypeptide.

In preferred embodiments the method includes: ascertaining the existence of at least one of: a deletion of one or more nucleotides from the 21617 or 55562 gene; an insertion of one or more nucleotides into the gene, a point mutation, e.g., a substitution of one or more nucleotides of the gene, a gross chromosomal rearrangement of the gene, e.g., a translocation, inversion, or deletion.

For example, detecting the genetic lesion can include: (i) providing a probe/primer including an oligonucleotide containing a region of nucleotide sequence which hybridizes to a sense or antisense sequence from SEQ ID NO:63 or SEQ ID NO:66, or naturally occurring mutants thereof or 5′ or 3′ flanking sequences naturally associated with the 21617 or 55562 gene; (ii) exposing the probe/primer to nucleic acid of the tissue; and detecting, by hybridization, e.g., in situ hybridization, of the probe/primer to the nucleic acid, the presence or absence of the genetic lesion.

In preferred embodiments detecting the misexpression includes ascertaining the existence of at least one of: an alteration in the level of a messenger RNA transcript of the 21617 or 55562 gene; the presence of a non-wild type splicing pattern of a messenger RNA transcript of the gene; or a non-wild type level of 21617 or 55562.

Methods of the invention can be used prenatally or to determine if a subject's offspring will be at risk for a disorder.

In preferred embodiments the method includes determining the structure of a 21617 or 55562 gene, an abnormal structure being indicative of risk for the disorder.

In preferred embodiments the method includes contacting a sample from the subject with an antibody to the 21617 or 55562 protein or a nucleic acid, which hybridizes specifically with the gene. These and other embodiments are discussed below.

Diagnostic and Prognostic Assays of 21617 and 55562

Diagnostic and prognostic assays of the invention include method for assessing the expression level of 21617 or 55562 molecules and for identifying variations and mutations in the sequence of 21617 or 55562 molecules.

Expression Monitoring and Profiling:

The presence, level, or absence of 21617 or 55562 protein or nucleic acid in a biological sample can be evaluated by obtaining a biological sample from a test subject and contacting the biological sample with a compound or an agent capable of detecting 21617 or 55562 protein or nucleic acid (e.g., mRNA, genomic DNA) that encodes 21617 or 55562 protein such that the presence of 21617 or 55562 protein or nucleic acid is detected in the biological sample. The term “biological sample” includes tissues, cells and biological fluids isolated from a subject, as well as tissues, cells and fluids present within a subject. A preferred biological sample is serum.

The level of expression of the 21617 or 55562 gene can be measured in a number of ways, including, but not limited to: measuring the mRNA encoded by the 21617 or 55562 genes; measuring the amount of protein encoded by the 21617 or 55562 genes; or measuring the activity of the protein encoded by the 21617 or 55562 genes.

The level of mRNA corresponding to the 21617 or 55562 gene in a cell can be determined both by in situ and by in vitro formats.

The isolated mRNA can be used in hybridization or amplification assays that include, but are not limited to, Southern or Northern analyses, polymerase chain reaction analyses and probe arrays. One preferred diagnostic method for the detection of mRNA levels involves contacting the isolated mRNA with a nucleic acid molecule (probe) that can hybridize to the mRNA encoded by the gene being detected. The nucleic acid probe can be, for example, a full-length 21617 or 55562 nucleic acid, such as the nucleic acid of SEQ ID NO:63 or SEQ ID NO:66, or a portion thereof, such as an oligonucleotide of at least 7, 15, 30, 50, 100, 250 or 500 nucleotides in length and sufficient to specifically hybridize under stringent conditions to 21617 or 55562 mRNA or genomic DNA. The probe can be disposed on an address of an array, e.g., an array described below. Other suitable probes for use in the diagnostic assays are described herein.

In one format, mRNA (or cDNA) is immobilized on a surface and contacted with the probes, for example by running the isolated mRNA on an agarose gel and transferring the mRNA from the gel to a membrane, such as nitrocellulose. In an alternative format, the probes are immobilized on a surface and the mRNA (or cDNA) is contacted with the probes, for example, in a two-dimensional gene chip array described below. A skilled artisan can adapt known mRNA detection methods for use in detecting the level of mRNA encoded by the 21617 or 55562 genes.

The level of mRNA in a sample that is encoded by one of 21617 or 55562 can be evaluated with nucleic acid amplification, e.g., by rtPCR (Mullis (1987) U.S. Pat. No. 4,683,202), ligase chain reaction (Barany (1991) Proc. Natl. Acad. Sci. USA 88:189-193), self sustained sequence replication (Guatelli et al., (1990) Proc. Natl. Acad. Sci. USA 87:1874-1878), transcriptional amplification system (Kwoh et al., (1989), Proc. Natl. Acad. Sci. USA 86:1173-1177), Q-Beta Replicase (Lizardi et al., (1988) Bio/Technology 6:1197), rolling circle replication (Lizardi et al., U.S. Pat. No. 5,854,033) or any other nucleic acid amplification method, followed by the detection of the amplified molecules using techniques known in the art. As used herein, amplification primers are defined as being a pair of nucleic acid molecules that can anneal to 5′ or 3′ regions of a gene (plus and minus strands, respectively, or vice-versa) and contain a short region in between. In general, amplification primers are from about 10 to 30 nucleotides in length and flank a region from about 50 to 200 nucleotides in length. Under appropriate conditions and with appropriate reagents, such primers permit the amplification of a nucleic acid molecule comprising the nucleotide sequence flanked by the primers.

For in situ methods, a cell or tissue sample can be prepared/processed and immobilized on a support, typically a glass slide, and then contacted with a probe that can hybridize to mRNA that encodes the 21617 or 55562 gene being analyzed.

In another embodiment, the methods further contacting a control sample with a compound or agent capable of detecting 21617 or 55562 mRNA, or genomic DNA, and comparing the presence of 21617 or 55562 mRNA or genomic DNA in the control sample with the presence of 21617 or 55562 mRNA or genomic DNA in the test sample. In still another embodiment, serial analysis of gene expression, as described in U.S. Pat. No. 5,695,937, is used to detect 21617 or 55562 transcript levels.

A variety of methods can be used to determine the level of protein encoded by 21617 or 55562. In general, these methods include contacting an agent that selectively binds to the protein, such as an antibody with a sample, to evaluate the level of protein in the sample. In a preferred embodiment, the antibody bears a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with a detectable substance. Examples of detectable substances are provided herein.

The detection methods can be used to detect 21617 or 55562 protein in a biological sample in vitro as well as in vivo. In vitro techniques for detection of 21617 or 55562 protein include enzyme linked immunosorbent assays (ELISAs), immunoprecipitations, immunofluorescence, enzyme immunoassay (EIA), radioimmunoassay (RIA), and Western blot analysis. In vivo techniques for detection of 21617 or 55562 protein include introducing into a subject a labeled anti-21617 or 55562 antibody. For example, the antibody can be labeled with a radioactive marker whose presence and location in a subject can be detected by standard imaging techniques. In another embodiment, the sample is labeled, e.g., biotinylated and then contacted to the antibody, e.g., an anti-21617 or 55562 antibody positioned on an antibody array (as described below). The sample can be detected, e.g., with avidin coupled to a fluorescent label.

In another embodiment, the methods further include contacting the control sample with a compound or agent capable of detecting 21617 or 55562 protein, and comparing the presence of 21617 or 55562 protein in the control sample with the presence of 21617 or 55562 protein in the test sample.

The invention also includes kits for detecting the presence of 21617 or 55562 in a biological sample. For example, the kit can include a compound or agent capable of detecting 21617 or 55562 protein or mRNA in a biological sample; and a standard. The compound or agent can be packaged in a suitable container. The kit can further comprise instructions for using the kit to detect 21617 or 55562 protein or nucleic acid.

For antibody-based kits, the kit can include: (1) a first antibody (e.g., attached to a solid support) which binds to a polypeptide corresponding to a marker of the invention; and, optionally, (2) a second, different antibody which binds to either the polypeptide or the first antibody and is conjugated to a detectable agent.

For oligonucleotide-based kits, the kit can include: (1) an oligonucleotide, e.g., a detectably labeled oligonucleotide, which hybridizes to a nucleic acid sequence encoding a polypeptide corresponding to a marker of the invention or (2) a pair of primers useful for amplifying a nucleic acid molecule corresponding to a marker of the invention. The kit can also includes a buffering agent, a preservative, or a protein stabilizing agent. The kit can also includes components necessary for detecting the detectable agent (e.g., an enzyme or a substrate). The kit can also contain a control sample or a series of control samples which can be assayed and compared to the test sample contained. Each component of the kit can be enclosed within an individual container and all of the various containers can be within a single package, along with instructions for interpreting the results of the assays performed using the kit.

The diagnostic methods described herein can identify subjects having, or at risk of developing, a disease or disorder associated with misexpressed or aberrant or unwanted 21617 or 55562 expression or activity. As used herein, the term “unwanted” includes an unwanted phenomenon involved in a biological response such as a cellular proliferation and/or differentiation disorder, metabolic disorder, kidney disorder, endothelial cell disorder, neural disorder, or viral disorder.

In one embodiment, a disease or disorder associated with aberrant or unwanted 21617 or 55562 expression or activity is identified. A test sample is obtained from a subject and 21617 or 55562 protein or nucleic acid (e.g., mRNA or genomic DNA) is evaluated, wherein the level, e.g., the presence or absence, of 21617 or 55562 protein or nucleic acid is diagnostic for a subject having or at risk of developing a disease or disorder associated with aberrant or unwanted 21617 or 55562 expression or activity. As used herein, a “test sample” refers to a biological sample obtained from a subject of interest, including a biological fluid (e.g., serum), cell sample, or tissue.

The prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant or unwanted 21617 or 55562 expression or activity. For example, such methods can be used to determine whether a subject can be effectively treated with an agent for a cellular proliferative and/or differentiative disorder, metabolic disorder, kidney disorder, endothelial cell disorder, neural disorder, or viral disorder.

In another aspect, the invention features a computer medium having a plurality of digitally encoded data records. Each data record includes a value representing the level of expression of 21617 or 55562 in a sample, and a descriptor of the sample. The descriptor of the sample can be an identifier of the sample, a subject from which the sample was derived (e.g., a patient), a diagnosis, or a treatment (e.g., a preferred treatment). In a preferred embodiment, the data record further includes values representing the level of expression of genes other than 21617 or 55562 (e.g., other genes associated with a 21617 or 55562-disorder, or other genes on an array). The data record can be structured as a table, e.g., a table that is part of a database such as a relational database (e.g., a SQL database of the Oracle or Sybase database environments).

Also featured is a method of evaluating a sample. The method includes providing a sample, e.g., from the subject, and determining a gene expression profile of the sample, wherein the profile includes a value representing the level of 21617 or 55562 expression. The method can further include comparing the value or the profile (i.e., multiple values) to a reference value or reference profile. The gene expression profile of the sample can be obtained by any of the methods described herein (e.g., by providing a nucleic acid from the sample and contacting the nucleic acid to an array). The method can be used to diagnose a cellular proliferation and/or differentiation disorders, metabolic disorders, kidney disorders, endothelial cell disorders, neural disorders, and viral disorders disorder in a subject wherein an increase or decrease in 21617 or 55562 expression is an indication that the subject has or is disposed to having such a disorder. For example, an increase in 21617 expression can be an indication that the subject has or is disposed to having a cellular proliferation and/or differentiation disorder, e.g., colon, colorectal, breast, lung, cervical, ovarian or liver cancer. The method can be used to monitor a treatment for a cellular proliferation and/or differentiation disorder, metabolic disorder, kidney disorder, endothelial cell disorder, neural disorder, or viral disorder in a subject. For example, the gene expression profile can be determined for a sample from a subject undergoing treatment. The profile can be compared to a reference profile or to a profile obtained from the subject prior to treatment or prior to onset of the disorder (see, e.g., Golub et al. (1999) Science 286:531).

In yet another aspect, the invention features a method of evaluating a test compound (see also, “Screening Assays”, above). The method includes providing a cell and a test compound; contacting the test compound to the cell; obtaining a subject expression profile for the contacted cell; and comparing the subject expression profile to one or more reference profiles. The profiles include a value representing the level of 21617 or 55562 expression. In a preferred embodiment, the subject expression profile is compared to a target profile, e.g., a profile for a normal cell or for desired condition of a cell. The test compound is evaluated favorably if the subject expression profile is more similar to the target profile than an expression profile obtained from an uncontacted cell.

In another aspect, the invention features, a method of evaluating a subject. The method includes: a) obtaining a sample from a subject, e.g., from a caregiver, e.g., a caregiver who obtains the sample from the subject; b) determining a subject expression profile for the sample. Optionally, the method further includes either or both of steps: c) comparing the subject expression profile to one or more reference expression profiles; and d) selecting the reference profile most similar to the subject reference profile. The subject expression profile and the reference profiles include a value representing the level of 21617 or 55562 expression. A variety of routine statistical measures can be used to compare two reference profiles. One possible metric is the length of the distance vector that is the difference between the two profiles. Each of the subject and reference profile is represented as a multi-dimensional vector, wherein each dimension is a value in the profile.

The method can further include transmitting a result to a caregiver. The result can be the subject expression profile, a result of a comparison of the subject expression profile with another profile, a most similar reference profile, or a descriptor of any of the aforementioned. The result can be transmitted across a computer network, e.g., the result can be in the form of a computer transmission, e.g., a computer data signal embedded in a carrier wave.

Also featured is a computer medium having executable code for effecting the following steps: receive a subject expression profile; access a database of reference expression profiles; and either i) select a matching reference profile most similar to the subject expression profile or ii) determine at least one comparison score for the similarity of the subject expression profile to at least one reference profile. The subject expression profile, and the reference expression profiles each include a value representing the level of 21617 or 55562 expression.

21617 and 55562 Arrays and uses Thereof

In another aspect, the invention features an array that includes a substrate having a plurality of addresses. At least one address of the plurality includes a capture probe that binds specifically to a 21617 or 55562 molecule (e.g., a 21617 or 55562 nucleic acid or a 21617 or 55562 polypeptide). The array can have a density of at least than 10, 50, 100, 200, 500, 1,000, 2,000, or 10,000 or more addresses/cm², and ranges between. In a preferred embodiment, the plurality of addresses includes at least 10, 100, 500, 1,000, 5,000, 10,000, 50,000 addresses. In a preferred embodiment, the plurality of addresses includes equal to or less than 10, 100, 500, 1,000, 5,000, 10,000, or 50,000 addresses. The substrate can be a two-dimensional substrate such as a glass slide, a wafer (e.g., silica or plastic), a mass spectroscopy plate, or a three-dimensional substrate such as a gel pad. Addresses in addition to address of the plurality can be disposed on the array.

In a preferred embodiment, at least one address of the plurality includes a nucleic acid capture probe that hybridizes specifically to a 21617 or 55562 nucleic acid, e.g., the sense or anti-sense strand. In one preferred embodiment, a subset of addresses of the plurality of addresses has a nucleic acid capture probe for 21617 or 55562. Each address of the subset can include a capture probe that hybridizes to a different region of a 21617 or 55562 nucleic acid. In another preferred embodiment, addresses of the subset include a capture probe for a 21617 or 55562 nucleic acid. Each address of the subset is unique, overlapping, and complementary to a different variant of 21617 or 55562 (e.g., an allelic variant, or all possible hypothetical variants). The array can be used to sequence 21617 or 55562 by hybridization (see, e.g., U.S. Pat. No. 5,695,940).

An array can be generated by various methods, e.g., by photolithographic methods (see, e.g., U.S. Pat. Nos. 5,143,854; 5,510,270; and 5,527,681), mechanical methods (e.g., directed-flow methods as described in U.S. Pat. No. 5,384,261), pin-based methods (e.g., as described in U.S. Pat. No. 5,288,514), and bead-based techniques (e.g., as described in PCT US/93/04145).

In another preferred embodiment, at least one address of the plurality includes a polypeptide capture probe that binds specifically to a 21617 or 55562 polypeptide or fragment thereof. The polypeptide can be a naturally-occurring interaction partner of 21617 or 55562 polypeptide. Preferably, the polypeptide is an antibody, e.g., an antibody described herein (see “Anti-21617 or 55562 Antibodies,” above), such as a monoclonal antibody or a single-chain antibody.

In another aspect, the invention features a method of analyzing the expression of 21617 or 55562. The method includes providing an array as described above; contacting the array with a sample and detecting binding of a 21617 or 55562-molecule (e.g., nucleic acid or polypeptide) to the array. In a preferred embodiment, the array is a nucleic acid array. Optionally the method further includes amplifying nucleic acid from the sample prior or during contact with the array.

In another embodiment, the array can be used to assay gene expression in a tissue to ascertain tissue specificity of genes in the array, particularly the expression of 21617 or 55562. If a sufficient number of diverse samples is analyzed, clustering (e.g., hierarchical clustering, k-means clustering, Bayesian clustering and the like) can be used to identify other genes which are co-regulated with 21617 or 55562. For example, the array can be used for the quantitation of the expression of multiple genes. Thus, not only tissue specificity, but also the level of expression of a battery of genes in the tissue is ascertained. Quantitative data can be used to group (e.g., cluster) genes on the basis of their tissue expression per se and level of expression in that tissue.

For example, array analysis of gene expression can be used to assess the effect of cell-cell interactions on 21617 or 55562 expression. A first tissue can be perturbed and nucleic acid from a second tissue that interacts with the first tissue can be analyzed. In this context, the effect of one cell type on another cell type in response to a biological stimulus can be determined, e.g., to monitor the effect of cell-cell interaction at the level of gene expression.

In another embodiment, cells are contacted with a therapeutic agent. The expression profile of the cells is determined using the array, and the expression profile is compared to the profile of like cells not contacted with the agent. For example, the assay can be used to determine or analyze the molecular basis of an undesirable effect of the therapeutic agent. If an agent is administered therapeutically to treat one cell type but has an undesirable effect on another cell type, the invention provides an assay to determine the molecular basis of the undesirable effect and thus provides the opportunity to co-administer a counteracting agent or otherwise treat the undesired effect. Similarly, even within a single cell type, undesirable biological effects can be determined at the molecular level. Thus, the effects of an agent on expression of other than the target gene can be ascertained and counteracted.

In another embodiment, the array can be used to monitor expression of one or more genes in the array with respect to time. For example, samples obtained from different time points can be probed with the array. Such analysis can identify and/or characterize the development of a 21617 or 55562-associated disease or disorder; and processes, such as a cellular transformation associated with a 21617 or 55562-associated disease or disorder. The method can also evaluate the treatment and/or progression of a 21617 or 55562-associated disease or disorder The array is also useful for ascertaining differential expression patterns of one or more genes in normal and abnormal cells. This provides a battery of genes (e.g., including 21617 or 55562) that could serve as a molecular target for diagnosis or therapeutic intervention.

In another aspect, the invention features an array having a plurality of addresses. Each address of the plurality includes a unique polypeptide. At least one address of the plurality has disposed thereon a 21617 or 55562 polypeptide or fragment thereof. Methods of producing polypeptide arrays are described in the art, e.g., in De Wildt et al. (2000). Nature Biotech. 18, 989-994; Lueking et al. (1999). Anal. Biochem. 270, 103-111; Ge, H. (2000). Nucleic Acids Res. 28, e3, I-VII; MacBeath, G., and Schreiber, S. L. (2000). Science 289, 1760-1763; and WO 99/51773A1. In a preferred embodiment, each addresses of the plurality has disposed thereon a polypeptide at least 60, 70, 80, 85, 90, 95 or 99% identical to a 21617 or 55562 polypeptide or fragment thereof. For example, multiple variants of a 21617 or 55562 polypeptide (e.g., encoded by allelic variants, site-directed mutants, random mutants, or combinatorial mutants) can be disposed at individual addresses of the plurality. Addresses in addition to the address of the plurality can be disposed on the array.

The polypeptide array can be used to detect a 21617 or 55562 binding compound, e.g., an antibody in a sample from a subject with specificity for a 21617 or 55562 polypeptide or the presence of a 21617 or 55562-binding protein or ligand.

The array is also useful for ascertaining the effect of the expression of a gene on the expression of other genes in the same cell or in different cells (e.g., ascertaining the effect of 21617 or 55562 expression on the expression of other genes). This provides, for example, for a selection of alternate molecular targets for therapeutic intervention if the ultimate or downstream target cannot be regulated.

In another aspect, the invention features a method of analyzing a plurality of probes. The method is useful, e.g., for analyzing gene expression. The method includes: providing a two dimensional array having a plurality of addresses, each address of the plurality being positionally distinguishable from each other address of the plurality having a unique capture probe, e.g., wherein the capture probes are from a cell or subject which express 21617 or 55562 or from a cell or subject in which a 21617 or 55562 mediated response has been elicited, e.g., by contact of the cell with 21617 or 55562 nucleic acid or protein, or administration to the cell or subject 21617 or 55562 nucleic acid or protein; providing a two dimensional array having a plurality of addresses, each address of the plurality being positionally distinguishable from each other address of the plurality, and each address of the plurality having a unique capture probe, e.g., wherein the capture probes are from a cell or subject which does not express 21617 or 55562 (or does not express as highly as in the case of the 21617 or 55562 positive plurality of capture probes) or from a cell or subject which in which a 21617 or 55562 mediated response has not been elicited (or has been elicited to a lesser extent than in the first sample); contacting the array with one or more inquiry probes (which is preferably other than a 21617 or 55562 nucleic acid, polypeptide, or antibody), and thereby evaluating the plurality of capture probes. Binding, e.g., in the case of a nucleic acid, hybridization with a capture probe at an address of the plurality, is detected, e.g., by signal generated from a label attached to the nucleic acid, polypeptide, or antibody.

In another aspect, the invention features a method of analyzing a plurality of probes or a sample. The method is useful, e.g., for analyzing gene expression. The method includes: providing a two dimensional array having a plurality of addresses, each address of the plurality being positionally distinguishable from each other address of the plurality having a unique capture probe, contacting the array with a first sample from a cell or subject which express or mis-express 21617 or 55562 or from a cell or subject in which a 21617 or 55562-mediated response has been elicited, e.g., by contact of the cell with 21617 or 55562 nucleic acid or protein, or administration to the cell or subject 21617 or 55562 nucleic acid or protein; providing a two dimensional array having a plurality of addresses, each address of the plurality being positionally distinguishable from each other address of the plurality, and each address of the plurality having a unique capture probe, and contacting the array with a second sample from a cell or subject which does not express 21617 or 55562 (or does not express as highly as in the case of the 21617 or 55562 positive plurality of capture probes) or from a cell or subject which in which a 21617 or 55562 mediated response has not been elicited (or has been elicited to a lesser extent than in the first sample); and comparing the binding of the first sample with the binding of the second sample. Binding, e.g., in the case of a nucleic acid, hybridization with a capture probe at an address of the plurality, is detected, e.g., by signal generated from a label attached to the nucleic acid, polypeptide, or antibody. The same array can be used for both samples or different arrays can be used. If different arrays are used the plurality of addresses with capture probes should be present on both arrays.

In another aspect, the invention features a method of analyzing 21617 or 55562, e.g., analyzing structure, function, or relatedness to other nucleic acid or amino acid sequences. The method includes: providing a 21617 or 55562 nucleic acid or amino acid sequence; comparing the 21617 or 55562 sequence with one or more preferably a plurality of sequences from a collection of sequences, e.g., a nucleic acid or protein sequence database; to thereby analyze 21617 or 55562.

Detection of 21617 and 55562 Variations or Mutations

The methods of the invention can also be used to detect genetic alterations in a 21617 or 55562 gene, thereby determining if a subject with the altered gene is at risk for a disorder characterized by misregulation in 21617 or 55562 protein activity or nucleic acid expression, such as a cellular proliferation and/or differentiation disorders, metabolic disorders, kidney disorders, endothelial cell disorders, neural disorders, and viral disorders In preferred embodiments, the methods include detecting, in a sample from the subject, the presence or absence of a genetic alteration characterized by at least one of an alteration affecting the integrity of a gene encoding a 21617 or 55562-protein, or the mis-expression of the 21617 or 55562 gene. For example, such genetic alterations can be detected by ascertaining the existence of at least one of 1) a deletion of one or more nucleotides from a 21617 or 55562 gene; 2) an addition of one or more nucleotides to a 21617 or 55562 gene; 3) a substitution of one or more nucleotides of a 21617 or 55562 gene, 4) a chromosomal rearrangement of a 21617 or 55562 gene; 5) an alteration in the level of a messenger RNA transcript of a 21617 or 55562 gene, 6) aberrant modification of a 21617 or 55562 gene, such as of the methylation pattern of the genomic DNA, 7) the presence of a non-wild type splicing pattern of a messenger RNA transcript of a 21617 or 55562 gene, 8) a non-wild type level of a 21617 or 55562-protein, 9) allelic loss of a 21617 or 55562 gene, and 10) inappropriate post-translational modification of a 21617 or 55562-protein.

An alteration can be detected without a probe/primer in a polymerase chain reaction, such as anchor PCR or RACE PCR, or, alternatively, in a ligation chain reaction (LCR), the latter of which can be particularly useful for detecting point mutations in the 21617 or 55562-gene. This method can include the steps of collecting a sample of cells from a subject, isolating nucleic acid (e.g., genomic, mRNA or both) from the sample, contacting the nucleic acid sample with one or more primers which specifically hybridize to a 21617 or 55562 gene under conditions such that hybridization and amplification of the 21617 or 55562-gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein. Alternatively, other amplification methods described herein or known in the art can be used.

In another embodiment, mutations in a 21617 or 55562 gene from a sample cell can be identified by detecting alterations in restriction enzyme cleavage patterns. For example, sample and control DNA is isolated, amplified (optionally), digested with one or more restriction endonucleases, and fragment length sizes are determined, e.g., by gel electrophoresis and compared. Differences in fragment length sizes between sample and control DNA indicates mutations in the sample DNA. Moreover, the use of sequence specific ribozymes (see, for example, U.S. Pat. No. 5,498,531) can be used to score for the presence of specific mutations by development or loss of a ribozyme cleavage site.

In other embodiments, genetic mutations in 21617 or 55562 can be identified by hybridizing a sample and control nucleic acids, e.g., DNA or RNA, two-dimensional arrays, e.g., chip based arrays. Such arrays include a plurality of addresses, each of which is positionally distinguishable from the other. A different probe is located at each address of the plurality. A probe can be complementary to a region of a 21617 or 55562 nucleic acid or a putative variant (e.g., allelic variant) thereof. A probe can have one or more mismatches to a region of a 21617 or 55562 nucleic acid (e.g., a destabilizing mismatch). The arrays can have a high density of addresses, e.g., can contain hundreds or thousands of oligonucleotides probes (Cronin, M. T. et al. (1996) Human Mutation 7: 244-255; Kozal, M. J. et al. (1996) Nature Medicine 2: 753-759). For example, genetic mutations in 21617 or 55562 can be identified in two-dimensional arrays containing light-generated DNA probes as described in Cronin, M. T. et al. supra. Briefly, a first hybridization array of probes can be used to scan through long stretches of DNA in a sample and control to identify base changes between the sequences by making linear arrays of sequential overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the 21617 or 55562 gene and detect mutations by comparing the sequence of the sample 21617 or 55562 with the corresponding wild-type (control) sequence. Automated sequencing procedures can be utilized when performing the diagnostic assays ((1995) Biotechniques 19:448), including sequencing by mass spectrometry.

Other methods for detecting mutations in the 21617 or 55562 gene include methods in which protection from cleavage agents is used to detect mismatched bases in RNA/RNA or RNA/DNA heteroduplexes (Myers et al. (1985) Science 230:1242; Cotton et al. (1988) Proc. Natl Acad Sci USA 85:4397; Saleeba et al. (1992) Methods Enzymol. 217:286-295).

In still another embodiment, the mismatch cleavage reaction employs one or more proteins that recognize mismatched base pairs in double-stranded DNA (so called “DNA mismatch repair” enzymes) in defined systems for detecting and mapping point mutations in 21617 or 55562 cDNAs obtained from samples of cells. For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1662; U.S. Pat. No. 5,459,039).

In other embodiments, alterations in electrophoretic mobility will be used to identify mutations in 21617 or 55562 genes. For example, single strand conformation polymorphism (SSCP) may be used to detect differences in electrophoretic mobility between mutant and wild type nucleic acids (Orita et al. (1989) Proc Natl. Acad. Sci USA: 86:2766, see also Cotton (1993) Mutat. Res. 285:125-144; and Hayashi (1992) Genet. Anal. Tech. Appl. 9:73-79). Single-stranded DNA fragments of sample and control 21617 or 55562 nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. (1991) Trends Genet 7:5).

In yet another embodiment, the movement of mutant or wild-type fragments in polyacrylamide gels containing a gradient of denaturant is assayed using denaturing gradient gel electrophoresis (DGGE) (Myers et al. (1985) Nature 313:495). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner (1987) Biophys Chem 265:12753).

Examples of other techniques for detecting point mutations include, but are not limited to, selective oligonucleotide hybridization, selective amplification, or selective primer extension (Saiki et al. (1986) Nature 324:163); Saiki et al. (1989) Proc. Natl. Acad. Sci USA 86:6230). A further method of detecting point mutations is the chemical ligation of oligonucleotides as described in Xu et al. ((2001) Nature Biotechnol. 19:148). Adjacent oligonucleotides, one of which selectively anneals to the query site, are ligated together if the nucleotide at the query site of the sample nucleic acid is complementary to the query oligonucleotide; ligation can be monitored, e.g., by fluorescent dyes coupled to the oligonucleotides.

Alternatively, allele specific amplification technology that depends on selective PCR amplification may be used in conjunction with the instant invention. Oligonucleotides used as primers for specific amplification may carry the mutation of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent, or reduce polymerase extension (Prossner (1993) Tibtech 11:238). In addition it may be desirable to introduce a novel restriction site in the region of the mutation to create cleavage-based detection (Gasparini et al. (1992) Mol. Cell Probes 6:1). It is anticipated that in certain embodiments amplification may also be performed using Taq ligase for amplification (Barany (1991) Proc. Natl. Acad. Sci USA 88:189). In such cases, ligation will occur only if there is a perfect match at the 3′ end of the 5′ sequence making it possible to detect the presence of a known mutation at a specific site by looking for the presence or absence of amplification.

In another aspect, the invention features a set of oligonucleotides. The set includes a plurality of oligonucleotides, each of which is at least partially complementary (e.g., at least 50%, 60%, 70%, 80%, 90%, 92%, 95%, 97%, 98%, or 99% complementary) to a 21617 or 55562 nucleic acid.

In a preferred embodiment the set includes a first and a second oligonucleotide. The first and second oligonucleotide can hybridize to the same or to different locations of SEQ ID NO:63 or SEQ ID NO:66 or the complement of SEQ ID NO:63 or SEQ ID NO:66. Different locations can be different but overlapping, or non-overlapping on the same strand. The first and second oligonucleotide can hybridize to sites on the same or on different strands.

The set can be useful, e.g., for identifying SNP's, or identifying specific alleles of 21617 or 55562. In a preferred embodiment, each oligonucleotide of the set has a different nucleotide at an interrogation position. In one embodiment, the set includes two oligonucleotides, each complementary to a different allele at a locus, e.g., a biallelic or polymorphic locus.

In another embodiment, the set includes four oligonucleotides, each having a different nucleotide (e.g., adenine, guanine, cytosine, or thymidine) at the interrogation position. The interrogation position can be a SNP or the site of a mutation. In another preferred embodiment, the oligonucleotides of the plurality are identical in sequence to one another (except for differences in length). The oligonucleotides can be provided with differential labels, such that an oligonucleotide that hybridizes to one allele provides a signal that is distinguishable from an oligonucleotide that hybridizes to a second allele. In still another embodiment, at least one of the oligonucleotides of the set has a nucleotide change at a position in addition to a query position, e.g., a destabilizing mutation to decrease the Tₘ of the oligonucleotide. In another embodiment, at least one oligonucleotide of the set has a non-natural nucleotide, e.g., inosine. In a preferred embodiment, the oligonucleotides are attached to a solid support, e.g., to different addresses of an array or to different beads or nanoparticles.

In a preferred embodiment the set of oligo nucleotides can be used to specifically amplify, e.g., by PCR, or detect, a 21617 or 55562 nucleic acid.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving a 21617 or 55562 gene.

Use of 21617 or 55562 Molecules as Surrogate Markers

The 21617 or 55562 molecules of the invention are also useful as markers of disorders or disease states, as markers for precursors of disease states, as markers for predisposition of disease states, as markers of drug activity, or as markers of the pharmacogenomic profile of a subject. Using the methods described herein, the presence, absence and/or quantity of the 21617 or 55562 molecules of the invention may be detected, and may be correlated with one or more biological states in vivo. For example, the 21617 or 55562 molecules of the invention may serve as surrogate markers for one or more disorders or disease states or for conditions leading up to disease states. As used herein, a “surrogate marker” is an objective biochemical marker which correlates with the absence or presence of a disease or disorder, or with the progression of a disease or disorder (e.g., with the presence or absence of a tumor). The presence or quantity of such markers is independent of the disease. Therefore, these markers may serve to indicate whether a particular course of treatment is effective in lessening a disease state or disorder. Surrogate markers are of particular use when the presence or extent of a disease state or disorder is difficult to assess through standard methodologies (e.g., early stage tumors), or when an assessment of disease progression is desired before a potentially dangerous clinical endpoint is reached (e.g., an assessment of cardiovascular disease may be made using cholesterol levels as a surrogate marker, and an analysis of HIV infection may be made using HIV RNA levels as a surrogate marker, well in advance of the undesirable clinical outcomes of myocardial infarction or fully-developed AIDS). Examples of the use of surrogate markers in the art include: Koomen et al. (2000) J. Mass. Spectrom. 35: 258-264; and James (1994) AIDS Treatment News Archive 209.

The 21617 or 55562 molecules of the invention are also useful as pharmacodynamic markers. As used herein, a “pharmacodynamic marker” is an objective biochemical marker which correlates specifically with drug effects. The presence or quantity of a pharmacodynamic marker is not related to the disease state or disorder for which the drug is being administered; therefore, the presence or quantity of the marker is indicative of the presence or activity of the drug in a subject. For example, a pharmacodynamic marker may be indicative of the concentration of the drug in a biological tissue, in that the marker is either expressed or transcribed or not expressed or transcribed in that tissue in relationship to the level of the drug. In this fashion, the distribution or uptake of the drug may be monitored by the pharmacodynamic marker. Similarly, the presence or quantity of the pharmacodynamic marker may be related to the presence or quantity of the metabolic product of a drug, such that the presence or quantity of the marker is indicative of the relative breakdown rate of the drug in vivo. Pharmacodynamic markers are of particular use in increasing the sensitivity of detection of drug effects, particularly when the drug is administered in low doses. Since even a small amount of a drug may be sufficient to activate multiple rounds of marker (e.g., a 21617 or 55562 marker) transcription or expression, the amplified marker may be in a quantity which is more readily detectable than the drug itself. Also, the marker may be more easily detected due to the nature of the marker itself; for example, using the methods described herein, anti-21617 or 55562 antibodies may be employed in an immune-based detection system for a 21617 or 55562 protein marker, or 21617 or 55562-specific radiolabeled probes may be used to detect a 21617 or 55562 mRNA marker. Furthermore, the use of a pharmacodynamic marker may offer mechanism-based prediction of risk due to drug treatment beyond the range of possible direct observations. Examples of the use of pharmacodynamic markers in the art include: Matsuda et al. U.S. Pat. No. 6,033,862; Hattis et al. (1991) Env. Health Perspect. 90: 229-238; Schentag (1999) Am. J. Health-Syst. Pharm. 56 Suppl. 3: S21-S24; and Nicolau (1999) Am, J Health-Syst. Pharm. 56 Suppl. 3: S16-S20.

The 21617 or 55562 molecules of the invention are also useful as pharmacogenomic markers. As used herein, a “pharmacogenomic marker” is an objective biochemical marker which correlates with a specific clinical drug response or susceptibility in a subject (see, e.g., McLeod et al. (1999) Eur. J. Cancer 35:1650-1652). The presence or quantity of the pharmacogenomic marker is related to the predicted response of the subject to a specific drug or class of drugs prior to administration of the drug. By assessing the presence or quantity of one or more pharmacogenomic markers in a subject, a drug therapy which is most appropriate for the subject, or which is predicted to have a greater degree of success, may be selected. For example, based on the presence or quantity of RNA, or protein (e.g., 21617 or 55562 protein or RNA) for specific tumor markers in a subject, a drug or course of treatment may be selected that is optimized for the treatment of the specific tumor likely to be present in the subject. Similarly, the presence or absence of a specific sequence mutation in 21617 or 55562 DNA may correlate 21617 or 55562 drug response. The use of pharmacogenomic markers therefore permits the application of the most appropriate treatment for each subject without having to administer the therapy.

Pharmaceutical Compositions of 21617 and 55562

The nucleic acid and polypeptides, fragments thereof, as well as anti-21617 or 55562 antibodies (also referred to herein as “active compounds”) of the invention can be incorporated into pharmaceutical compositions. Such compositions typically include the nucleic acid molecule, protein, or antibody and a pharmaceutically acceptable carrier. As used herein the language “pharmaceutically acceptable carrier” includes solvents, dispersion media, coatings, antibacterial and antifungal agents, isotonic and absorption delaying agents, and the like, compatible with pharmaceutical administration. Supplementary active compounds can also be incorporated into the compositions.

A pharmaceutical composition is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor ELTM (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

As defined herein, a therapeutically effective amount of protein or polypeptide (i.e., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The protein or polypeptide can be administered one time per week for between about 1 to 10 weeks, preferably between 2 to 8 weeks, more preferably between about 3 to 7 weeks, and even more preferably for about 4, 5, or 6 weeks. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of a protein, polypeptide, or antibody can include a single treatment or, preferably, can include a series of treatments.

For antibodies, the preferred dosage is 0.1 mg/kg of body weight (generally 10 mg/kg to 20 mg/kg). If the antibody is to act in the brain, a dosage of 50 mg/kg to 100 mg/kg is usually appropriate. Generally, partially human antibodies and fully human antibodies have a longer half-life within the human body than other antibodies. Accordingly, lower dosages and less frequent administration is often possible. Modifications such as lipidation can be used to stabilize antibodies and to enhance uptake and tissue penetration (e.g., into the brain). A method for lipidation of antibodies is described by Cruikshank et al. ((1997) J. Acquired Immune Deficiency Syndromes and Human Retrovirology 14:193).

The present invention encompasses agents which modulate expression or activity. An agent may, for example, be a small molecule. For example, such small molecules include, but are not limited to, peptides, peptidomimetics (e.g., peptoids), amino acids, amino acid analogs, polynucleotides, polynucleotide analogs, nucleotides, nucleotide analogs, organic or inorganic compounds (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

Exemplary doses include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram. It is furthermore understood that appropriate doses of a small molecule depend upon the potency of the small molecule with respect to the expression or activity to be modulated. When one or more of these small molecules is to be administered to an animal (e.g., a human) in order to modulate expression or activity of a polypeptide or nucleic acid of the invention, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

An antibody (or fragment thereof) may be conjugated to a therapeutic moiety such as a cytotoxin, a therapeutic agent or a radioactive ion. A cytotoxin or cytotoxic agent includes any agent that is detrimental to cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, puromycin, maytansinoids, e.g., maytansinol (see U.S. Pat. No. 5,208,020), CC-1065 (see U.S. Pat. Nos. 5,475,092, 5,585,499, 5,846,545) and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, CC-1065, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine, vinblastine, taxol and maytansinoids). Radioactive ions include, but are not limited to iodine, yttrium and praseodymium.

The conjugates of the invention can be used for modifying a given biological response, the drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, α-interferon, β-interferon, nerve growth factor, platelet derived growth factor, tissue plasminogen activator; or, biological response modifiers such as, for example, lymphokines, interleukin-1 (“IL-1”), interleukin-2 (“IL-2”), interleukin-6 (“IL-6”), granulocyte macrophase colony stimulating factor (“GM-CSF”), granulocyte colony stimulating factor (“G-CSF”), or other growth factors. Alternatively, an antibody can be conjugated to a second antibody to form an antibody heteroconjugate as described by Segal in U.S. Pat. No. 4,676,980.

The nucleic acid molecules of the invention can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

The pharmaceutical compositions can be included in a container, pack, or dispenser together with instructions for administration.

Methods of Treatment for 21617 and 55562

The present invention provides for both prophylactic and therapeutic methods of treating a subject at risk of (or susceptible to) a disorder or having a disorder associated with aberrant or unwanted 21617 or 55562 expression or activity. As used herein, the term “treatment” is defined as the application or administration of a therapeutic agent to a patient, or application or administration of a therapeutic agent to an isolated tissue or cell line from a patient, who has a disease, a symptom of disease or a predisposition toward a disease, with the purpose to cure, heal, alleviate, relieve, alter, remedy, ameliorate, improve or affect the disease, the symptoms of disease or the predisposition toward disease. A therapeutic agent includes, but is not limited to, small molecules, peptides, antibodies, ribozymes and antisense oligonucleotides.

With regards to both prophylactic and therapeutic methods of treatment, such treatments may be specifically tailored or modified, based on knowledge obtained from the field of pharmacogenomics. “Pharmacogenomics”, as used herein, refers to the application of genomics technologies such as gene sequencing, statistical genetics, and gene expression analysis to drugs in clinical development and on the market. More specifically, the term refers the study of how a patient's genes determine his or her response to a drug (e.g., a patient's “drug response phenotype”, or “drug response genotype”.) Thus, another aspect of the invention provides methods for tailoring an individual's prophylactic or therapeutic treatment with either the 21617 or 55562 molecules of the present invention or 21617 or 55562 modulators according to that individual's drug response genotype. Pharmacogenomics allows a clinician or physician to target prophylactic or therapeutic treatments to patients who will most benefit from the treatment and to avoid treatment of patients who will experience toxic drug-related side effects.

In one aspect, the invention provides a method for preventing in a subject, a disease or condition associated with an aberrant or unwanted 21617 or 55562 expression or activity, by administering to the subject a 21617 or 55562 or an agent which modulates 21617 or 55562 expression or at least one 21617 or 55562 activity. Subjects at risk for a disease which is caused or contributed to by aberrant or unwanted 21617 or 55562 expression or activity can be identified by, for example, any or a combination of diagnostic or prognostic assays as described herein. Administration of a prophylactic agent can occur prior to the manifestation of symptoms characteristic of the 21617 or 55562 aberrance, such that a disease or disorder is prevented or, alternatively, delayed in its progression. Depending on the type of 21617 or 55562 aberrance, for example, a 21617 or 55562, 21617 or 55562 agonist or 21617 or 55562 antagonist agent can be used for treating the subject. The appropriate agent can be determined based on screening assays described herein.

It is possible that some 21617 or 55562 disorders can be caused, at least in part, by an abnormal level of gene product, or by the presence of a gene product exhibiting abnormal activity. As such, the reduction in the level and/or activity of such gene products would bring about the amelioration of disorder symptoms.

The 21617 or 55562 molecules can act as novel diagnostic targets and therapeutic agents for controlling one or more of cellular proliferative and/or differentiative disorders, metabolic disorders, viral disorders, kidney disorders, endothelial cell disorders, neural disorders (e.g., brain disorders), and viral disorders, as discussed above, as well as disorders associated with bone metabolism, immune disorders, cardiovascular disorders, liver disorders, and pain disorders.

Disorders associated with bone metabolism refers to disorders in the formation or degeneration of bone structures, e.g., bone formation, bone resorption, etc., which may ultimately affect the concentrations in serum of calcium and phosphate. The disorders may involve bone cells, e.g. osteoclasts and osteoblasts, and may result in bone formation or degeneration. Disorders effecting bone metabolism can also involve monocytes and mononuclear phagocytes, which differentiate to form osteoclasts. Accordingly, 21617 or 55562 molecules that modulate the production of bone cells can influence bone formation and degeneration, and thus may be used to treat bone disorders. Examples of such disorders include, but are not limited to, osteoporosis, osteodystrophy, osteomalacia, rickets, osteitis fibrosa cystica, renal osteodystrophy, osteosclerosis, anti-convulsant treatment, osteopenia, fibrogenesis-imperfecta ossium, secondary hyperparathyrodism, hypoparathyroidism, hyperparathyroidism, cirrhosis, obstructive jaundice, drug induced metabolism, medullary carcinoma, chronic renal disease, rickets, sarcoidosis, glucocorticoid antagonism, malabsorption syndrome, steatorrhea, tropical sprue, idiopathic hypercalcemia and milk fever.

Examples of immune disorders or diseases include, but are not limited to, autoimmune diseases (including, for example, diabetes mellitus, arthritis (including rheumatoid arthritis, juvenile rheumatoid arthritis, osteoarthritis, psoriatic arthritis), multiple sclerosis, encephalomyelitis, myasthenia gravis, systemic lupus erythematosis, autoimmune thyroiditis, dermatitis (including atopic dermatitis and eczematous dermatitis), psoriasis, Sjogren's Syndrome, Crohn's disease, aphthous ulcer, iritis, conjunctivitis, keratoconjunctivitis, ulcerative colitis, asthma, allergic asthma, cutaneous lupus erythematosus, scleroderma, vaginitis, proctitis, drug eruptions, leprosy reversal reactions, erythema nodosum leprosum, autoimmune uveitis, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis, Wegener's granulomatosis, chronic active hepatitis, Stevens-Johnson syndrome, idiopathic sprue, lichen planus, Graves' disease, sarcoidosis, primary biliary cirrhosis, uveitis posterior, and interstitial lung fibrosis), graft-versus-host disease, cases of transplantation, and allergy such as, atopic allergy.

As used herein, disorders involving the heart, or “cardiovascular disease” or a “cardiovascular disorder” includes a disease or disorder which affects the cardiovascular system, e.g., the heart, the blood vessels, and/or the blood. A cardiovascular disorder can be caused by an imbalance in arterial pressure, a malfunction of the heart, or an occlusion of a blood vessel, e.g., by a thrombus. A cardiovascular disorder includes, but is not limited to disorders such as arteriosclerosis, atherosclerosis, cardiac hypertrophy, ischemia reperfusion injury, restenosis, arterial inflammation, vascular wall remodeling, ventricular remodeling, rapid ventricular pacing, coronary microembolism, tachycardia, bradycardia, pressure overload, aortic bending, coronary artery ligation, vascular heart disease, valvular disease, including but not limited to, valvular degeneration caused by calcification, rheumatic heart disease, endocarditis, or complications of artificial valves; atrial fibrillation, long-QT syndrome, congestive heart failure, sinus node dysfunction, angina, heart failure, hypertension, atrial fibrillation, atrial flutter, pericardial disease, including but not limited to, pericardial effusion and pericarditis; cardiomyopathies, e.g., dilated cardiomyopathy or idiopathic cardiomyopathy, myocardial infarction, coronary artery disease, coronary artery spasm, ischemic disease, arrhythmia, sudden cardiac death, and cardiovascular developmental disorders (e.g., arteriovenous malformations, arteriovenous fistulae, raynaud's syndrome, neurogenic thoracic outlet syndrome, causalgia/reflex sympathetic dystrophy, hemangioma, aneurysm, cavernous angioma, aortic valve stenosis, atrial septal defects, atrioventricular canal, coarctation of the aorta, ebsteins anomaly, hypoplastic left heart syndrome, interruption of the aortic arch, mitral valve prolapse, ductus arteriosus, patent foramen ovale, partial anomalous pulmonary venous return, pulmonary atresia with ventricular septal defect, pulmonary atresia without ventricular septal defect, persistance of the fetal circulation, pulmonary valve stenosis, single ventricle, total anomalous pulmonary venous return, transposition of the great vessels, tricuspid atresia, truncus arteriosus, ventricular septal defects). A cardiovasular disease or disorder can also include an endothelial cell disorder and a hematological disorder.

A hematological disorder can include thrombosis. Thrombosis can result from platelet dysfunction, e.g., seen in myocardial infarction, angina, hypertension, lipid disorders, diabetes mellitus; myelodysplastic syndromes; myeloproliferative syndromes (including polycythemia vera and thombocythemia); thrombotic thrombocytopenic purpuras; HIV-induced platelet disorders (AIDS-Thrombocytopenia); heparin induced thrombocytopenia; mural cell alterations/interactions leading to platelet aggregation/degranulation, vascular endothelial cell activation/injury, monocyte/macrophage extravasation and smooth muscle cell proliferation; autoimmune disorders such as, but not limited to vasculitis, antiphospholipid syndromes, systemic lupus erythromatosis; inflammatory diseases, such as, but not limited to immune activation; graft vs. host disease; radiation induced hypercoagulation; clotting factor dysregulation either hereditary (autosomal dominant or recessive) such as, but not limited to clotting factor pathways including protein C/S, Anti-thrombin III deficiency, and the Factor V Leiden mutation or acquired such as but not limited to autoimmune, cancer-associated and drug-induced dysregulation of clotting factors.

Disorders which may be treated or diagnosed by methods described herein include, but are not limited to, disorders associated with an accumulation in the liver of fibrous tissue, such as that resulting from an imbalance between production and degradation of the extracellular matrix accompanied by the collapse and condensation of preexisting fibers. The methods described herein can be used to diagnose or treat hepatocellular necrosis or injury induced by a wide variety of agents including processes which disturb homeostasis, such as an inflammatory process, tissue damage resulting from toxic injury or altered hepatic blood flow, and infections (e.g., bacterial, viral and parasitic). For example, the methods can be used for the early detection of hepatic injury, such as portal hypertension or hepatic fibrosis. In addition, the methods can be employed to detect liver fibrosis attributed to inborn errors of metabolism, for example, fibrosis resulting from a storage disorder such as Gaucher's disease (lipid abnormalities) or a glycogen storage disease, A1-antitrypsin deficiency; a disorder mediating the accumulation (e.g., storage) of an exogenous substance, for example, hemochromatosis (iron-overload syndrome) and copper storage diseases (Wilson's disease), disorders resulting in the accumulation of a toxic metabolite (e.g., tyrosinemia, fructosemia and galactosemia) and peroxisomal disorders (e.g., Zellweger syndrome). Additionally, the methods described herein may be useful for the early detection and treatment of liver injury associated with the administration of various chemicals or drugs, such as for example, methotrexate, isonizaid, oxyphenisatin, methyldopa, chlorpromazine, tolbutamide or alcohol, or which represents a hepatic manifestation of a vascular disorder such as obstruction of either the intrahepatic or extrahepatic bile flow or an alteration in hepatic circulation resulting, for example, from chronic heart failure, veno-occlusive disease, portal vein thrombosis or Budd-Chiari syndrome.

Additionally, 21617 or 55562 may play an important role in the regulation of pain disorders. Examples of pain disorders include, but are not limited to, pain response elicited during various forms of tissue injury, e.g., inflammation, infection, and ischemia, usually referred to as hyperalgesia (described in, for example, Fields, H. L. (1987) Pain, New York:McGraw-Hill); pain associated with musculoskeletal disorders, e.g., joint pain; tooth pain; headaches; pain associated with surgery; pain related to irritable bowel syndrome; or chest pain.

As discussed, successful treatment of 21617 or 55562 disorders can be brought about by techniques that serve to inhibit the expression or activity of target gene products. For example, compounds, e.g., an agent identified using an assays described above, that proves to exhibit negative modulatory activity, can be used in accordance with the invention to prevent and/or ameliorate symptoms of 21617 or 55562 disorders. Such molecules can include, but are not limited to peptides, phosphopeptides, small organic or inorganic molecules, or antibodies (including, for example, polyclonal, monoclonal, humanized, anti-idiotypic, chimeric or single chain antibodies, and Fab, F(ab′)₂ and Fab expression library fragments, scFV molecules, and epitope-binding fragments thereof).

Further, antisense and ribozyme molecules that inhibit expression of the target gene can also be used in accordance with the invention to reduce the level of target gene expression, thus effectively reducing the level of target gene activity. Still further, triple helix molecules can be utilized in reducing the level of target gene activity. Antisense, ribozyme and triple helix molecules are discussed above.

It is possible that the use of antisense, ribozyme, and/or triple helix molecules to reduce or inhibit mutant gene expression can also reduce or inhibit the transcription (triple helix) and/or translation (antisense, ribozyme) of mRNA produced by normal target gene alleles, such that the concentration of normal target gene product present can be lower than is necessary for a normal phenotype. In such cases, nucleic acid molecules that encode and express target gene polypeptides exhibiting normal target gene activity can be introduced into cells via gene therapy method. Alternatively, in instances in that the target gene encodes an extracellular protein, it can be preferable to co-administer normal target gene protein into the cell or tissue in order to maintain the requisite level of cellular or tissue target gene activity.

Another method by which nucleic acid molecules may be utilized in treating or preventing a disease characterized by 21617 or 55562 expression is through the use of aptamer molecules specific for 21617 or 55562 protein. Aptamers are nucleic acid molecules having a tertiary structure which permits them to specifically bind to protein ligands (see, e.g., Osborne, et al. (1997) Curr. Opin. Chem Biol. 1: 5-9; and Patel, D. J. (1997) Curr Opin Chem Biol 1:32-46). Since nucleic acid molecules may in many cases be more conveniently introduced into target cells than therapeutic protein molecules may be, aptamers offer a method by which 21617 or 55562 protein activity may be specifically decreased without the introduction of drugs or other molecules which may have pluripotent effects.

Antibodies can be generated that are both specific for target gene product and that reduce target gene product activity. Such antibodies may, therefore, by administered in instances whereby negative modulatory techniques are appropriate for the treatment of 21617 or 55562 disorders. For a description of antibodies, see the Antibody section above.

In circumstances wherein injection of an animal or a human subject with a 21617 or 55562 protein or epitope for stimulating antibody production is harmful to the subject, it is possible to generate an immune response against 21617 or 55562 through the use of anti-idiotypic antibodies (see, for example, Herlyn, D. (1999) Ann Med 31:66-78; and Bhattacharya-Chatterjee, M., and Foon, K. A. (1998) Cancer Treat Res. 94:51-68). If an anti-idiotypic antibody is introduced into a mammal or human subject, it should stimulate the production of anti-anti-idiotypic antibodies, which should be specific to the 21617 or 55562 protein. Vaccines directed to a disease characterized by 21617 or 55562 expression may also be generated in this fashion.

In instances where the target antigen is intracellular and whole antibodies are used, internalizing antibodies may be preferred. Lipofectin or liposomes can be used to deliver the antibody or a fragment of the Fab region that binds to the target antigen into cells. Where fragments of the antibody are used, the smallest inhibitory fragment that binds to the target antigen is preferred. For example, peptides having an amino acid sequence corresponding to the Fv region of the antibody can be used. Alternatively, single chain neutralizing antibodies that bind to intracellular target antigens can also be administered. Such single chain antibodies can be administered, for example, by expressing nucleotide sequences encoding single-chain antibodies within the target cell population (see e.g., Marasco et al. (1993) Proc. Natl. Acad. Sci. USA 90:7889-7893).

The identified compounds that inhibit target gene expression, synthesis and/or activity can be administered to a patient at therapeutically effective doses to prevent, treat or ameliorate 21617 or 55562 disorders. A therapeutically effective dose refers to that amount of the compound sufficient to result in amelioration of symptoms of the disorders. Toxicity and therapeutic efficacy of such compounds can be determined by standard pharmaceutical procedures as described above.

The data obtained from the cell culture assays and animal studies can be used in formulating a range of dosage for use in humans. The dosage of such compounds lies preferably within a range of circulating concentrations that include the ED₅₀ with little or no toxicity. The dosage can vary within this range depending upon the dosage form employed and the route of administration utilized. For any compound used in the method of the invention, the therapeutically effective dose can be estimated initially from cell culture assays. A dose can be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma can be measured, for example, by high performance liquid chromatography.

Another example of determination of effective dose for an individual is the ability to directly assay levels of “free” and “bound” compound in the serum of the test subject. Such assays may utilize antibody mimics and/or “biosensors” that have been created through molecular imprinting techniques. The compound which is able to modulate 21617 or 55562 activity is used as a template, or “imprinting molecule”, to spatially organize polymerizable monomers prior to their polymerization with catalytic reagents. The subsequent removal of the imprinted molecule leaves a polymer matrix which contains a repeated “negative image” of the compound and is able to selectively rebind the molecule under biological assay conditions. A detailed review of this technique can be seen in Ansell, R. J. et al. (1996) Current Opinion in Biotechnology 7:89-94 and in Shea, K. J. (1994) Trends in Polymer Science 2:166-173. Such “imprinted” affinity matrixes are amenable to ligand-binding assays, whereby the immobilized monoclonal antibody component is replaced by an appropriately imprinted matrix. An example of the use of such matrixes in this way can be seen in Vlatakis, G. et al. (1993) Nature 361:645-647. Through the use of isotope-labeling, the “free” concentration of compound which modulates the expression or activity of 21617 or 55562 can be readily monitored and used in calculations of IC₅₀.

Such “imprinted” affinity matrixes can also be designed to include fluorescent groups whose photon-emitting properties measurably change upon local and selective binding of target compound. These changes can be readily assayed in real time using appropriate fiberoptic devices, in turn allowing the dose in a test subject to be quickly optimized based on its individual IC₅₀. An rudimentary example of such a “biosensor” is discussed in Kriz, D. et al. (1995) Analytical Chemistry 67:2142-2144.

Another aspect of the invention pertains to methods of modulating 21617 or 55562 expression or activity for therapeutic purposes. Accordingly, in an exemplary embodiment, the modulatory method of the invention involves contacting a cell with a 21617 or 55562 or agent that modulates one or more of the activities of 21617 or 55562 protein activity associated with the cell. An agent that modulates 21617 or 55562 protein activity can be an agent as described herein, such as a nucleic acid or a protein, a naturally-occurring target molecule of a 21617 or 55562 protein (e.g., a 21617 or 55562 substrate or receptor), a 21617 or 55562 antibody, a 21617 or 55562 agonist or antagonist, a peptidomimetic of a 21617 or 55562 agonist or antagonist, or other small molecule.

In one embodiment, the agent stimulates one or 21617 or 55562 activities. Examples of such stimulatory agents include active 21617 or 55562 protein and a nucleic acid molecule encoding 21617 or 55562. In another embodiment, the agent inhibits one or more 21617 or 55562 activities. Examples of such inhibitory agents include antisense 21617 or 55562 nucleic acid molecules, anti-21617 or 55562 antibodies, and 21617 or 55562 inhibitors. These modulatory methods can be performed in vitro (e.g., by culturing the cell with the agent) or, alternatively, in vivo (e.g., by administering the agent to a subject). As such, the present invention provides methods of treating an individual afflicted with a disease or disorder characterized by aberrant or unwanted expression or activity of a 21617 or 55562 protein or nucleic acid molecule. In one embodiment, the method involves administering an agent (e.g., an agent identified by a screening assay described herein), or combination of agents that modulates (e.g., up regulates or down regulates) 21617 or 55562 expression or activity. In another embodiment, the method involves administering a 21617 or 55562 protein or nucleic acid molecule as therapy to compensate for reduced, aberrant, or unwanted 21617 or 55562 expression or activity.

Stimulation of 21617 or 55562 activity is desirable in situations in which 21617 or 55562 is abnormally downregulated and/or in which increased 21617 or 55562 activity is likely to have a beneficial effect. For example, stimulation of 21617 or 55562 activity is desirable in situations in which a 21617 or 55562 is downregulated and/or in which increased 21617 or 55562 activity is likely to have a beneficial effect. Likewise, inhibition of 21617 or 55562 activity is desirable in situations in which 21617 or 55562 is abnormally upregulated and/or in which decreased 21617 or 55562 activity is likely to have a beneficial effect.

21617 and 55562 Pharmacogenomics

The 21617 or 55562 molecules of the present invention, as well as agents, or modulators which have a stimulatory or inhibitory effect on 21617 or 55562 activity (e.g., 21617 or 55562 gene expression) as identified by a screening assay described herein can be administered to individuals to treat (prophylactically or therapeutically) 21617 or 55562 associated disorders (e.g., cellular proliferation and/or differentiation disorders, metabolic disorders, kidney disorders, endothelial cell disorders, neural disorders, or viral disorders) associated with aberrant or unwanted 21617 or 55562 activity. In conjunction with such treatment, pharmacogenomics (i.e., the study of the relationship between an individual's genotype and that individual's response to a foreign compound or drug) may be considered. Differences in metabolism of therapeutics can lead to severe toxicity or therapeutic failure by altering the relation between dose and blood concentration of the pharmacologically active drug. Thus, a physician or clinician may consider applying knowledge obtained in relevant pharmacogenomics studies in determining whether to administer a 21617 or 55562 molecule or 21617 or 55562 modulator as well as tailoring the dosage and/or therapeutic regimen of treatment with a 21617 or 55562 molecule or 21617 or 55562 modulator.

Pharmacogenomics deals with clinically significant hereditary variations in the response to drugs due to altered drug disposition and abnormal action in affected persons. See, for example, Eichelbaum, M. et al. (1996) Clin. Exp. Pharmacol. Physiol. 23:983-985 and Linder, M. W. et al. (1997) Clin. Chem. 43:254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare genetic defects or as naturally-occurring polymorphisms. For example, glucose-6-phosphate dehydrogenase deficiency (G6PD) is a common inherited enzymopathy in which the main clinical complication is haemolysis after ingestion of oxidant drugs (anti-malarials, sulfonamides, analgesics, nitrofurans) and consumption of fava beans.

One pharmacogenomics approach to identifying genes that predict drug response, known as “a genome-wide association”, relies primarily on a high-resolution map of the human genome consisting of already known gene-related markers (e.g., a “bi-allelic” gene marker map which consists of 60,000-100,000 polymorphic or variable sites on the human genome, each of which has two variants.) Such a high-resolution genetic map can be compared to a map of the genome of each of a statistically significant number of patients taking part in a Phase II/III drug trial to identify markers associated with a particular observed drug response or side effect. Alternatively, such a high resolution map can be generated from a combination of some ten-million known single nucleotide polymorphisms (SNPs) in the human genome. As used herein, a “SNP” is a common alteration that occurs in a single nucleotide base in a stretch of DNA. For example, a SNP may occur once per every 1000 bases of DNA. A SNP may be involved in a disease process, however, the vast majority may not be disease-associated. Given a genetic map based on the occurrence of such SNPs, individuals can be grouped into genetic categories depending on a particular pattern of SNPs in their individual genome. In such a manner, treatment regimens can be tailored to groups of genetically similar individuals, taking into account traits that may be common among such genetically similar individuals.

Alternatively, a method termed the “candidate gene approach,” can be utilized to identify genes that predict drug response. According to this method, if a gene that encodes a drug's target is known (e.g., a 21617 or 55562 protein of the present invention), all common variants of that gene can be fairly easily identified in the population and it can be determined if having one version of the gene versus another is associated with a particular drug response.

Alternatively, a method termed the “gene expression profiling,” can be utilized to identify genes that predict drug response. For example, the gene expression of an animal dosed with a drug (e.g., a 21617 or 55562 molecule or 21617 or 55562 modulator of the present invention) can give an indication whether gene pathways related to toxicity have been turned on.

Information generated from more than one of the above pharmacogenomics approaches can be used to determine appropriate dosage and treatment regimens for prophylactic or therapeutic treatment of an individual. This knowledge, when applied to dosing or drug selection, can avoid adverse reactions or therapeutic failure and thus enhance therapeutic or prophylactic efficiency when treating a subject with a 21617 or 55562 molecule or 21617 or 55562 modulator, such as a modulator identified by one of the exemplary screening assays described herein.

The present invention further provides methods for identifying new agents, or combinations, that are based on identifying agents that modulate the activity of one or more of the gene products encoded by one or more of the 21617 or 55562 genes of the present invention, wherein these products may be associated with resistance of the cells to a therapeutic agent. Specifically, the activity of the proteins encoded by the 21617 or 55562 genes of the present invention can be used as a basis for identifying agents for overcoming agent resistance. By blocking the activity of one or more of the resistance proteins, target cells, e.g., human cells, will become sensitive to treatment with an agent that the unmodified target cells were resistant to.

Monitoring the influence of agents (e.g., drugs) on the expression or activity of a 21617 or 55562 protein can be applied in clinical trials. For example, the effectiveness of an agent determined by a screening assay as described herein to increase 21617 or 55562 gene expression, protein levels, or upregulate 21617 or 55562 activity, can be monitored in clinical trials of subjects exhibiting decreased 21617 or 55562 gene expression, protein levels, or downregulated 21617 or 55562 activity. Alternatively, the effectiveness of an agent determined by a screening assay to decrease 21617 or 55562 gene expression, protein levels, or downregulate 21617 or 55562 activity, can be monitored in clinical trials of subjects exhibiting increased 21617 or 55562 gene expression, protein levels, or upregulated 21617 or 55562 activity. In such clinical trials, the expression or activity of a 21617 or 55562 gene, and preferably, other genes that have been implicated in, for example, a 21617 or 55562-associated disorder can be used as a “read out” or markers of the phenotype of a particular cell.

21617 or 55562 Informatics

The sequence of a 21617 or 55562 molecule is provided in a variety of media to facilitate use thereof. A sequence can be provided as a manufacture, other than an isolated nucleic acid or amino acid molecule, which contains a 21617 or 55562. Such a manufacture can provide a nucleotide or amino acid sequence, e.g., an open reading frame, in a form which allows examination of the manufacture using means not directly applicable to examining the nucleotide or amino acid sequences, or a subset thereof, as they exists in nature or in purified form. The sequence information can include, but is not limited to, 21617 or 55562 full-length nucleotide and/or amino acid sequences, partial nucleotide and/or amino acid sequences, polymorphic sequences including single nucleotide polymorphisms (SNPs), epitope sequence, and the like. In a preferred embodiment, the manufacture is a machine-readable medium, e.g., a magnetic, optical, chemical or mechanical information storage device.

As used herein, “machine-readable media” refers to any medium that can be read and accessed directly by a machine, e.g., a digital computer or analogue computer. Non-limiting examples of a computer include a desktop PC, laptop, mainframe, server (e.g., a web server, network server, or server farm), handheld digital assistant, pager, mobile telephone, and the like. The computer can be stand-alone or connected to a communications network, e.g., a local area network (such as a VPN or intranet), a wide area network (e.g., an Extranet or the Internet), or a telephone network (e.g., a wireless, DSL, or ISDN network). Machine-readable media include, but are not limited to: magnetic storage media, such as floppy discs, hard disc storage medium, and magnetic tape; optical storage media such as CD-ROM; electrical storage media such as RAM, ROM, EPROM, EEPROM, flash memory, and the like; and hybrids of these categories such as magnetic/optical storage media.

A variety of data storage structures are available to a skilled artisan for creating a machine-readable medium having recorded thereon a nucleotide or amino acid sequence of the present invention. The choice of the data storage structure will generally be based on the means chosen to access the stored information. In addition, a variety of data processor programs and formats can be used to store the nucleotide sequence information of the present invention on computer readable medium. The sequence information can be represented in a word processing text file, formatted in commercially-available software such as WordPerfect and Microsoft Word, or represented in the form of an ASCII file, stored in a database application, such as DB2, Sybase, Oracle, or the like. The skilled artisan can readily adapt any number of data processor structuring formats (e.g., text file or database) in order to obtain computer readable medium having recorded thereon the nucleotide sequence information of the present invention.

In a preferred embodiment, the sequence information is stored in a relational database (such as Sybase or Oracle). The database can have a first table for storing sequence (nucleic acid and/or amino acid sequence) information. The sequence information can be stored in one field (e.g., a first column) of a table row and an identifier for the sequence can be store in another field (e.g., a second column) of the table row. The database can have a second table, e.g., storing annotations. The second table can have a field for the sequence identifier, a field for a descriptor or annotation text (e.g., the descriptor can refer to a functionality of the sequence, a field for the initial position in the sequence to which the annotation refers, and a field for the ultimate position in the sequence to which the annotation refers. Non-limiting examples for annotation to nucleic acid sequences include polymorphisms (e.g., SNP's) translational regulatory sites and splice junctions. Non-limiting examples for annotations to amino acid sequence include polypeptide domains, e.g., a domain described herein; active sites and other functional amino acids; and modification sites.

By providing the nucleotide or amino acid sequences of the invention in computer readable form, the skilled artisan can routinely access the sequence information for a variety of purposes. For example, one skilled in the art can use the nucleotide or amino acid sequences of the invention in computer readable form to compare a target sequence or target structural motif with the sequence information stored within the data storage means. A search is used to identify fragments or regions of the sequences of the invention which match a particular target sequence or target motif. The search can be a BLAST search or other routine sequence comparison, e.g., a search described herein.

Thus, in one aspect, the invention features a method of analyzing 21617 or 55562, e.g., analyzing structure, function, or relatedness to one or more other nucleic acid or amino acid sequences. The method includes: providing a 21617 or 55562 nucleic acid or amino acid sequence; comparing the 21617 or 55562 sequence with a second sequence, e.g., one or more preferably a plurality of sequences from a collection of sequences, e.g., a nucleic acid or protein sequence database to thereby analyze 21617 or 55562. The method can be performed in a machine, e.g., a computer, or manually by a skilled artisan.

The method can include evaluating the sequence identity between a 21617 or 55562 sequence and a database sequence. The method can be performed by accessing the database at a second site, e.g., over the Internet.

As used herein, a “target sequence” can be any DNA or amino acid sequence of six or more nucleotides or two or more amino acids. A skilled artisan can readily recognize that the longer a target sequence is, the less likely a target sequence will be present as a random occurrence in the database. Typical sequence lengths of a target sequence are from about 10 to 100 amino acids or from about 30 to 300 nucleotide residues. However, it is well recognized that commercially important fragments, such as sequence fragments involved in gene expression and protein processing, may be of shorter length.

Computer software is publicly available which allows a skilled artisan to access sequence information provided in a computer readable medium for analysis and comparison to other sequences. A variety of known algorithms are disclosed publicly and a variety of commercially available software for conducting search means are and can be used in the computer-based systems of the present invention. Examples of such software include, but are not limited to, MacPattern (EMBL), BLASTN and BLASTX (NCBI).

Thus, the invention features a method of making a computer readable record of a sequence of a 21617 or 55562 sequence which includes recording the sequence on a computer readable matrix. In a preferred embodiment the record includes one or more of the following: identification of an ORF; identification of a domain, region, or site; identification of the start of transcription; identification of the transcription terminator; the full length amino acid sequence of the protein, or a mature form thereof; the 5′ end of the translated region.

In another aspect, the invention features, a method of analyzing a sequence. The method includes: providing a 21617 or 55562 sequence, or record, in machine-readable form; comparing a second sequence to the 21617 or 55562 sequence; thereby analyzing a sequence.

Comparison can include comparing to sequences for sequence identity or determining if one sequence is included within the other, e.g., determining if the 21617 or 55562 sequence includes a sequence being compared. In a preferred embodiment the 21617 or 55562 or second sequence is stored on a first computer, e.g., at a first site and the comparison is performed, read, or recorded on a second computer, e.g., at a second site. E.g., the 21617 or 55562 or second sequence can be stored in a public or proprietary database in one computer, and the results of the comparison performed, read, or recorded on a second computer. In a preferred embodiment the record includes one or more of the following: identification of an ORF; identification of a domain, region, or site; identification of the start of transcription; identification of the transcription terminator; the full length amino acid sequence of the protein, or a mature form thereof; the 5′ end of the translated region.

In another aspect, the invention provides a machine-readable medium for holding instructions for performing a method for determining whether a subject has a 21617 or 55562-associated disease or disorder or a pre-disposition to a 21617 or 55562-associated disease or disorder, wherein the method comprises the steps of determining 21617 or 55562 sequence information associated with the subject and based on the 21617 or 55562 sequence information, determining whether the subject has a 21617 or 55562-associated disease or disorder or a pre-disposition to a 21617 or 55562-associated disease or disorder and/or recommending a particular treatment for the disease, disorder or pre-disease condition.

The invention further provides in an electronic system and/or in a network, a method for determining whether a subject has a 21617 or 55562-associated disease or disorder or a pre-disposition to a disease associated with a 21617 or 55562 wherein the method comprises the steps of determining 21617 or 55562 sequence information associated with the subject, and based on the 21617 or 55562 sequence information, determining whether the subject has a 21617 or 55562-associated disease or disorder or a pre-disposition to a 21617 or 55562-associated disease or disorder, and/or recommending a particular treatment for the disease, disorder or pre-disease condition. In a preferred embodiment, the method further includes the step of receiving information, e.g., phenotypic or genotypic information, associated with the subject and/or acquiring from a network phenotypic information associated with the subject. The information can be stored in a database, e.g., a relational database. In another embodiment, the method further includes accessing the database, e.g., for records relating to other subjects, comparing the 21617 or 55562 sequence of the subject to the 21617 or 55562 sequences in the database to thereby determine whether the subject as a 21617 or 55562-associated disease or disorder, or a pre-disposition for such.

The present invention also provides in a network, a method for determining whether a subject has a 21617 or 55562 associated disease or disorder or a pre-disposition to a 21617 or 55562-associated disease or disorder associated with 21617 or 55562, said method comprising the steps of receiving 21617 or 55562 sequence information from the subject and/or information related thereto, receiving phenotypic information associated with the subject, acquiring information from the network corresponding to 21617 or 55562 and/or corresponding to a 21617 or 55562-associated disease or disorder (e.g., a cellular proliferation and/or differentiation disorder, metabolic disorder, kidney disorder, endothelial cell disorder, neural disorder, or viral disorder), and based on one or more of the phenotypic information, the 21617 or 55562 information (e.g., sequence information and/or information related thereto), and the acquired information, determining whether the subject has a 21617 or 55562-associated disease or disorder or a pre-disposition to a 21617 or 55562-associated disease or disorder. The method may further comprise the step of recommending a particular treatment for the disease, disorder or pre-disease condition.

The present invention also provides a method for determining whether a subject has a 21617 or 55562-associated disease or disorder or a pre-disposition to a 21617 or 55562-associated disease or disorder, said method comprising the steps of receiving information related to 21617 or 55562 (e.g., sequence information and/or information related thereto), receiving phenotypic information associated with the subject, acquiring information from the network related to 21617 or 55562 and/or related to a 21617 or 55562-associated disease or disorder, and based on one or more of the phenotypic information, the 21617 or 55562 information, and the acquired information, determining whether the subject has a 21617 or 55562-associated disease or disorder or a pre-disposition to a 21617 or 55562-associated disease or disorder. The method may further comprise the step of recommending a particular treatment for the disease, disorder or pre-disease condition.

This invention is further illustrated by the following examples that should not be construed as limiting. The contents of all references, patents and published patent applications cited throughout this application are incorporated herein by reference.

This invention is further illustrated by the following examples that should not be construed as limiting. The contents of all references, patents and published patent applications cited throughout this application are incorporated herein by reference.

### BACKGROUND OF THE 23566, 33489, AND 57779 INVENTION

Carboxypeptidases

Peptidases are divided into exopeptidases, that act only near a terminus of a polypeptide chain, and endopeptidases, that act internally. Exopeptidases (3.4.11-19) act either at the N-terminus (aminopeptidases) or at the C-terminus (carboxypeptidases; 3.4.16-18). The carboxypeptidases are grouped on the basis of catalytic mechanism: serine-type carboxypeptidases, metallocarboxypeptidases (3.4.17) and the cysteine-type carboxypeptidases. Other exopeptidases are specific for dipeptides (dipeptidases) or remove terminal residues that are substituted, cyclized or linked by isopeptide bonds (peptide linkages other than those of α-carboxyl to α-amino groups) (omega peptidases).

Carboxypeptidase A is a representative example of a metallocarboxypeptidase that catalyzes the hydrolysis of the peptide bonds of proteins sequentially from the C-terminus. In vertebrates, carboxypeptidase A is secreted from the pancreas into the small intestine as a zymogen (i.e., an inactive precursor). After activation, carboxypeptidase A participates in the digestion of proteins. Carboxypeptidase A will remove any C-terminus amino acid, while carboxypeptidase B, for example, is specific for terminal lysines or arginines.

Carboxypeptidase A contains a Zn²⁺ ion, which forms a ligand to the imidazole groups of histidine residues 69 and 196 and to the carboxylate group of glutamate residue 72. The fourth ligand in this tetrahedral complex is either a water molecule or a carbonyl group of the substrate. The action of carboxypeptidase A can be divided into three steps. In the first step, after the zinc ion forms a complex with a carbonyl group of the substrate and polarizes the carbon-oxygen bond, the carbonyl carbon is attacked by water in a reaction that is assisted by the carboxylate side chain of glutamate 270. As a result, a tetrahedral intermediate forms. In the second step, the tetrahedral intermediate rapidly ionizes to give a dianion intermediate. In the third step, the C—N bond of the substrate is cleaved, and the tetrahedral intermediate collapses.

Scramblases

The plasma membrane separates the cytoplasm from the extracellular environment and is composed of lipids arranged in a bilayer having a hydrophobic interior. The two layers are referred to as the inner and outer leaflets. Since these leaflets face, alternatively, the extracellular milieu and the cytoplasm, they can have different compositions and functions. For example, the plasma membrane of erythrocytes and platelets have an asymmetric distribution of amino phospholipids. In these cells, phosphatidyl serine and phosphatidylethanolamine are almost exclusively in the inner membrane leaflet whereas neutral polar phospholipids, such as phosphatidylcholine and sphingomyelin are on the outer leaflet.

However, in response to a variety of events including cell activation, e.g., by Ca²⁺ signals, injury, and apoptosis, the asymmetric composition of the plasma membrane can be disrupted. This resorting of plasma membrane phospholipids is mediated by a scramblase enzyme, which “scrambles” the contents of the two membrane leaflets. In the cardiovascular system, this event has dramatic consequences. Amino phospholipids are presented on the outer membrane leaflet where they are recognized by enzymes in the coagulation and complement systems. In addition, reticuloendothelial cells are stimulated to clear injured and apoptotic cells. Thus, scramblase enzymes are able to transduce an intracellular signal, e.g., Ca²⁺ activation, to the extracellular environment by presenting certain phospholipids on the outer leaflet of the plasma membrane.

The importance of scramblase activity is exemplified by Scott syndrome, a genetic disorder in which blood clotting is defective. In afflicted individuals, platelets and other blood cells are unable to activate the protease cascade of the coagulation system. The defect has been demonstrated to be the result of a failure in scramblase activity platelets as phosphatidylserine is not mobilized to the outer membrane leaflet (see e.g., Bevers et al. (1995) Blood 86:1983).

Protocadherins

Cadherins utilize a Ca²⁺-dependent mechanism for mediating cell-cell adhesion. Extracellular cadherin domains preferentially interact with the extracellular domains of the same cadherin on another cell. Because of this binding specificity for the same type, cadherin interactions are described as “homotypic”. Cadherins provide cells with in a tissue with a molecular addressing system that organizes both the physical integrity and the differentiative identity of the tissue. Cadherin proteins can mediate tissue identity during normal organismal development. Moreover, a number of cadherins are involved in the regulation of cell proliferation.

Among cadherin family members are a subclass of cadherins termed, “protocadherins.”

These proteins have at least six extracellular cadherin domains. However, they lack the cytoplasmic domain and prosegment typical of conventional cadherins (see, e.g., Sano et al. (1993) EMBO 12:2249-2256). Protocadherins can have multiple transmembrane domains, and a signal peptide. Typically, protocadherins mediate calcium dependent cell aggregation, as demonstrated by transfection of L cells with the protocadherin Pcdh2 (Obata et al. (1995) J Cell Sci. 108:3765-3773).

Carboxypeptidases, scramblases, and protocadherins have all been implicated in human disease. Consequently, the isolation and characterization of additional carboxypeptidases, scramblases, and protocadherins will provide novel reagents for the treatment or prevention of disease, as well as new targets for the development of drugs that can be used to treat or prevent disease.

### SUMMARY OF THE 23566, 33489, AND 57779 INVENTION

The present invention is based, in part, on the discovery of novel carboxypeptidase, scramblase, and protocadherin family members, referred to herein as “23566”, “33489”, and “57779”, respectively. The nucleotide sequence of a cDNA encoding 23566 is recited in SEQ ID NO:73; the nucleotide sequence of a cDNA encoding 33489 is recited in SEQ ID NO:76; and the nucleotide sequence of a cDNA encoding 57779 is recited in SEQ ID NO:79 (see Example 10). The amino acid sequence of a 23566, 33489, or 57779 polypeptide is shown in SEQ ID NO:74,, SEQ ID NO:77, and SEQ ID NO:80, respectively, while the nucleotide sequences of the coding regions are depicted in SEQ ID NO:75, SEQ ID NO:78, and SEQ ID NO:81, respectively.

Accordingly, in one aspect, the invention features a nucleic acid molecule that encodes a 23566, 33489, or 57779 protein or polypeptide, e.g., a biologically active portion of the 23566, 33489, or 57779 protein. In a preferred embodiment the isolated nucleic acid molecule encodes a polypeptide having the amino acid sequence of SEQ ID NO:74, SEQ ID NO:77, or SEQ ID NO:80. In other embodiments, the invention provides isolated 23566, 33489, or 57779 nucleic acid molecules having the nucleotide sequence shown in SEQ ID NO:73, SEQ ID NO:75, SEQ ID NO:76, SEQ ID NO:78, SEQ ID NO:79, or SEQ ID NO:81, or the sequence of a DNA insert of a plasmid deposited with an ATCC Acce Number as described herein. In still other embodiments, the invention provides nucleic acid molecules that are substantially identical (e.g., naturally occurring allelic variants) to the nucleotide sequence shown in SEQ ID NO:73, SEQ ID NO:75, SEQ ID NO:76, SEQ ID NO:78, SEQ ID NO:79, or SEQ ID NO:81, or the sequence of a DNA insert of a plasmid deposited with an ATCC Accession Number as described herein. In other embodiments, the invention provides a nucleic acid molecule which hybridizes under a stringency condition described herein to a nucleic acid molecule comprising the nucleotide sequence of SEQ ID NO:73, SEQ ID NO:75, SEQ ID NO:76, SEQ ID NO:78, SEQ ID NO:79, or SEQ ID NO:81, or the sequence of a DNA insert of a plasmid deposited with an ATCC Accession Number as described herein, wherein the nucleic acid encodes a full length 23566, 33489, or 57779 protein or an active fragment thereof.

In a related aspect, the invention further provides nucleic acid constructs that include a 23566, 33489, or 57779 nucleic acid molecule described herein. In certain embodiments, the nucleic acid molecules of the invention are operatively linked to native or heterologous regulatory sequences. Also included, are vectors and host cells containing the 23566, 33489, or 57779 nucleic acid molecules of the invention e.g., vectors and host cells suitable for producing 23566, 33489, or 57779 nucleic acid molecules and polypeptides.

In another related aspect, the invention provides nucleic acid fragments suitable as primers or hybridization probes for the detection of 23566, 33489, or 57779-encoding nucleic acids.

In still another related aspect, isolated nucleic acid molecules that are antisense to a 23566, 33489, or 57779 encoding nucleic acid molecule are provided.

In another aspect, the invention features, 23566, 33489, or 57779 polypeptides, and biologically active or antigenic fragments thereof that are useful, e.g., as reagents or targets in assays applicable to treatment and diagnosis of 23566, 33489, or 57779-mediated or related disorders. In another embodiment, the invention provides 23566, 33489, or 57779 polypeptides having a 23566, 33489, or 57779 activity. Preferred polypeptides are 23566, 33489, or 57779 proteins including at least one carboxypeptidase, scramblase, or protocadherin domain, and, preferably, having a 23566, 33489, or 57779 activity, e.g., a 23566, 33489, or 57779 activity as described herein.

In other embodiments, the invention provides 23566, 33489, or 57779 polypeptides, e.g., a 23566, 33489, or 57779 polypeptide having the amino acid sequence shown in SEQ ID NO:74, SEQ ID NO:77, OR SEQ ID NO:80 or the amino acid sequence encoded by the cDNA insert of a plasmid deposited with an ATCC Accession Number as described herein; an amino acid sequence that is substantially identical to the amino acid sequence shown in SEQ ID NO:74, SEQ ID NO:77, OR SEQ ID NO:80 or the amino acid sequence encoded by the cDNA insert of a plasmid deposited with an ATCC Accession Number as described herein; or an amino acid sequence encoded by a nucleic acid molecule having a nucleotide sequence which hybridizes under a stringency condition described herein to a nucleic acid molecule comprising the nucleotide sequence of SEQ ID NO:73, SEQ ID NO:75, SEQ ID NO:76, SEQ ID NO:78, SEQ ID NO:79, or SEQ ID NO:81, or the sequence of the DNA insert of a plasmid deposited with an ATCC Accession Number as described herein, wherein the nucleic acid encodes a full length 23566, 33489, or 57779 protein or an active fragment thereof.

In a related aspect, the invention provides 23566, 33489, or 57779 polypeptides or fragments operatively linked to non-23566, 33489, or 57779 polypeptides to form fusion proteins.

In another aspect, the invention features antibodies and antigen-binding fragments thereof, that react with, or more preferably specifically bind 23566, 33489, or 57779 polypeptides or fragments thereof, e.g., an extracellular domain of an 23566, 33489, or 57779 polypeptide.

In another aspect, the invention provides methods of screening for compounds that modulate the expression or activity of the 23566, 33489, or 57779 polypeptides or nucleic acids.

In still another aspect, the invention provides a process for modulating 23566, 33489, or 57779 polypeptide or nucleic acid expression or activity, e.g. using the screened compounds. In certain embodiments, the methods involve treatment of conditions related to aberrant activity or expression of the 23566, 33489, or 57779 polypeptides or nucleic acids, such as conditions involving inflammatory disorders, neurological disorders, cardiovascular disorders (e.g., disorders of the heart and/or blood vessels), blood clotting disorders, or cellular proliferation or differentiation disorders.

In some embodiments, the compound (i.e., screened compound) is an inhibitor of a 23566, 33489, or 57779 polypeptide. Preferably, the inhibitor is chosen from a peptide, a phosphopeptide, a small organic molecule, a small inorganic molecule and an antibody (e.g., an antibody conjugated to a therapeutic moiety). In some embodiments, the compound is an inhibitor of a 23566, 33489, or 57779 nucleic acid, e.g., an antisense, a ribozyme, or a triple helix molecule.

In some embodiments, the compound (i.e., screened compound) is an activator of a 23566, 33489, or 57779 polypeptide. Preferably, the inhibitor is chosen from a peptide, a phosphopeptide, a small organic molecule, a small inorganic molecule and an antibody. In some embodiments, the compound is a transcriptional activator that stimulates the expression of a 23566, 33489, or 57779 nucleic acid, e.g., by directly or indirectly modulating the activity of a nuclear transcription factor.

In some embodiments, the compound is administered (e.g., to cells or to a subject) in combination with a second compound, e.g., a known therapeutic agent. Such a compound could be, e.g., a therapeutic agent used to treat inflammatory disorders, neurological disorders, cardiovascular disorders (e.g., disorders of the heart and/or blood vessels), blood clotting disorders, or cellular proliferation or differentiation disorders.

In another aspect, the invention features methods for treating or preventing a disorder characterized by aberrant cellular proliferation or differentiation of a 23566, 33489, or 57779-expressing cell, in a subject. Preferably, the method includes administering to the subject (e.g., a mammal, e.g., a human) an effective amount of a compound (e.g., a compound identified using the methods described herein) that modulates the activity, or expression, of the 23566, 33489, or 57779 polypeptide or nucleic acid. In a preferred embodiment, the disorder is a cancerous or pre-cancerous condition.

In a further aspect, the invention provides methods for evaluating the efficacy of a treatment of a disorder, e.g., an inflammatory disorder, neurological disorder, cardiovascular disorder (e.g., a disorder of the heart and/or blood vessels), blood clotting disorder, or cellular proliferation or differentiation disorder. The method includes: treating a subject, e.g., a patient or an animal, with a protocol under evaluation (e.g., treating a subject with one or more of: a known therapeutic agent, and/or a compound identified using the methods described herein); and evaluating the expression of a 23566, 33489, or 57779 nucleic acid or polypeptide before and after treatment. A change, e.g., a decrease or increase, in the level of a 23566, 33489, or 57779 nucleic acid (e.g., mRNA) or polypeptide after treatment, relative to the level of expression before treatment, is indicative of the efficacy of the treatment of the disorder. The level of 23566, 33489, or 57779 nucleic acid or polypeptide expression can be detected by any method described herein.

In a preferred embodiment, the evaluating step includes obtaining a sample (e.g., a tissue sample, e.g., a biopsy, or a fluid sample) from the subject, before and after treatment and comparing the level of expressing of a 23566, 33489, or 57779 nucleic acid (e.g., mRNA) or polypeptide before and after treatment.

In another aspect, the invention provides methods for evaluating the efficacy of a therapeutic or prophylactic agent (e.g., an anti-neoplastic agent). The method includes: contacting a sample with an agent (e.g., a known therapeutic agent or a compound identified using the methods described herein) and, evaluating the expression of 23566, 33489, or 57779 nucleic acid or polypeptide in the sample before and after the contacting step. A change, e.g., a decrease or increase, in the level of 23566, 33489, or 57779 nucleic acid (e.g., mRNA) or polypeptide in the sample obtained after the contacting step, relative to the level of expression in the sample before the contacting step, is indicative of the efficacy of the agent. The level of 23566, 33489, or 57779 nucleic acid or polypeptide expression can be detected by any method described herein. In a preferred embodiment, the sample includes cells obtained from the blood (e.g., hematopoietic cells, e.g., white blood cells or red blood cells), a neural tissue, a cardiovascular tissue (e.g., heart, endothelial, or smooth muscle cells), or a cancerous tissue.

The invention also provides assays for determining the activity of or the presence or absence of 23566, 33489, or 57779 polypeptides or nucleic acid molecules in a biological sample, including for disease diagnosis.

In further aspect, the invention provides assays for determining the presence or absence of a genetic alteration in a 23566, 33489, or 57779 polypeptide or nucleic acid molecule, including for disease diagnosis.

In another aspect, the invention features a two dimensional array having a plurality of addresses, each address of the plurality being positionally distinguishable from each other address of the plurality, and each address of the plurality having a unique capture probe, e.g., a nucleic acid or peptide sequence. At least one address of the plurality has a capture probe that recognizes a 23566, 33489, or 57779 molecule. In one embodiment, the capture probe is a nucleic acid, e.g., a probe complementary to a 23566, 33489, or 57779 nucleic acid sequence. In another embodiment, the capture probe is a polypeptide, e.g., an antibody specific for 23566, 33489, or 57779 polypeptides. Also featured is a method of analyzing a sample by contacting the sample to the aforementioned array and detecting binding of the sample to the array.

Other features and advantages of the invention will be apparent from the following detailed description, and from the claims.

### DETAILED DESCRIPTION OF 23566, 33489, AND 57779

Human 23566

The human 23566 sequence (see SEQ ID NO:73, as recited in Example 10), which is approximately 1603 nucleotides long including untranslated regions, contains a predicted methionine-initiated coding sequence of about 1332 nucleotides, including the termination codon (SEQ ID NO:75). The coding sequence encodes a 444 amino acid protein (see SEQ ID NO:74, as recited in Example 10). The human 23566 protein of SEQ ID NO:74 includes an amino-terminal hydrophobic amino acid sequence, consistent with a signal sequence, of about 68 amino acids (from amino acid 1 to about amino acid 68 of SEQ ID NO:74), which upon cleavage results in the production of a mature protein form.

Human 23566 contains the following regions or other structural features:

a zinc carboxypeptidase domain (PFAM Accession Number PF00246) located at about amino acid residues 180 to 443 of SEQ ID NO:74;

a zinc carboxypeptidase zinc-binding region 1 signature motif (PS00132) located at about amino acid residues 229 to 251 of SEQ ID NO:74;

a predicted signal peptide located at about amino acid residues 1 to 68 of SEQ ID NO:74;

3 predicted N-glycosylation sites (PS00001) at about amino acids 304 to 307, 317 to 320 and 381 to 384 of SEQ ID NO:74;

5 predicted Protein Kinase C phosphorylation sites (PS00005) at about amino acid residues 46 to 48, 194 to 196, 291 to 293, 354 to 356 and 423 to 425 of SEQ ID NO:74;

5 predicted Casein Kinase II phosphorylation sites (PS00006) located at about amino acid residues 341 to 344, 350 to 353, 354 to 357, 412 to 415 and 423 to 426 of SEQ ID NO:74;

1 predicted Tyrosine kinase phosphorylation site (PS00007) located at about amino acid residues 132 to 139 of SEQ ID NO:74;

5 predicted N-myristylation sites (PS00008) located at about amino acid residues 14 to 19, 305 to 310, 324 to 329, 393 to 398 and 432 to 437 of SEQ ID NO:74; and

1 predicted Amidation site (PS00009) located at about amino acid residues 37 to 40 of SEQ ID NO:74.

For general information regarding PFAM identifiers, PS prefix and PF prefix domain identification numbers, refer to Sonnhammer et al. (1997) Protein 28:405-420 and http://www.psc.edu/general/software/packages/pfam/pfam.html.

A plasmid containing the nucleotide sequence encoding human 23566 (clone “Fbh23566FL”) was deposited with American Type Culture Collection (ATCC), 10801 University Boulevard, Manassas, Va. 20110-2209, on ______ and assigned Accession Number ______. This deposit will be maintained under the terms of the Budapest Treaty on the International Recognition of the Deposit of Microorganisms for the Purposes of Patent Procedure. This deposit was made merely as a convenience for those of skill in the art and is not an admission that a deposit is required under 35 U.S.C. §112.

Human 33489

The human 33489 sequence (see SEQ ID NO:76, as recited in Example 10), which is approximately 1748 nucleotides long including untranslated regions, contains a predicted methionine-initiated coding sequence of about 888 nucleotides, including the termination codon (SEQ ID NO:78). The coding sequence encodes a 295 amino acid protein (see SEQ ID NO:77, as recited in Example 10).

Human 33489 contains the following regions or other structural features:

a scramblase domain (ProDom Accession Number PD006852; Release 2000.1) located at about amino acid residues 103 to 285 of SEQ ID NO:77;

a proline rich potential SH3-domain binding motif located at about amino acid residues 12 to 19 of SEQ ID NO:77;

one predicted transmembrane domain located at about aino acid residues 266 to 282 of SEQ ID NO:77;

one predicted amino terminal cytoplasmic domain located at about amino acid residues 1 to 265 of SEQ ID NO:77;

one predicted carboxy terminal extracellular domain located at about amino acid residues 283 to 295 of SEQ ID NO:77;

one predicted glycosaminoglycan attachment site (PS00002) located at about amino acid residues 110 to 113 of SEQ ID NO:77;

five predicted casein kinase II phosphorylation sites (PS00006) located at about amino acid residues 74 to 77, 195 to 198, 220 to 223, 226 to 229, and 249 to 252 of SEQ ID NO:77; and

five predicted N-myristylation sites (PS00008) located at about amino acid residues 71 to 76, 113 to 118, 177 to 182, 217 to 222, and 287 to 292 of SEQ ID NO:77.

For general information regarding PFAM identifiers, PS prefix and PF prefix domain identification numbers, refer to Sonnhammer et al. (1997) Protein 28:405-420 and http://www.psc.edu/general/software/packages/pfam/pfam.html.

A plasmid containing the nucleotide sequence encoding human 33489 (clone “Fbh33489FL”) was deposited with American Type Culture Collection (ATCC), 10801 University Boulevard, Manassas, Va. 20110-2209, on ______ and assigned Accession Number ______. This deposit will be maintained under the terms of the Budapest Treaty on the International Recognition of the Deposit of Microorganisms for the Purposes of Patent Procedure. This deposit was made merely as a convenience for those of skill in the art and is not an admission that a deposit is required under 35 U.S.C. §112.

Human 57779

The human 57779 sequence (see SEQ ID NO:79, as recited in Example 10), which is approximately 2634 nucleotides long including untranslated regions, contains a predicted methionine-initiated coding sequence of about 2622 nucleotides, including the termination codon (SEQ ID NO:81). The coding sequence encodes a 873 amino acid protein (see SEQ ID NO:80, as recited in Example 10). The human 57779 protein of SEQ ID NO:80 includes an amino-terminal hydrophobic amino acid sequence, consistent with a signal sequence, of about 17 amino acids (from amino acid 8 to about amino acid 25 of SEQ ID NO:80). Cleavage and processing of human 57779 results in production of the mature protein form, of approximately 847 amino acid residues in length (from about amino acid 26 to amino acid 873 of SEQ ID NO:80).

Human 57779 contains the following regions or other structural features:

six cadherin domains (PFAM Accession Number PF00028) located at about amino acid residues 25 to 120, 134 to 229, 243 to 337, 354 to 444, 458 to 554, and 573 to 663 of SEQ ID NO:80;

five cadherin extracellular domain signature motifs (PS00232), located at about amino acid residues 117 to 127, 226 to 236, 334 to 344, 441 to 451, and 551 to 561 of SEQ ID NO:80;

a signal peptide located at about amino acid residues 8 to 25 of SEQ ID NO:80;

two predicted transmembrane domains locate at about amino acid residues 677 to 701, and 835 to 859 of SEQ ID NO:80;

two predicted extracellular domains locate at about amino acid residues 26 to 676 and 860 to 873 of SEQ ID NO:80;

one predicted intracellular domain locate at about amino acid residues 702 to 835 of SEQ ID NO:80;

eleven predicted protein kinase C phosphorylation sites (PS00005) located at about amino acid residues 96 to 98, 205 to 207, 225 to 227, 374 to 376, 435 to 437, 476 to 478, 548 to 550, 626 to 628, 633 to 635, 728 to 730, and 749 to 751 of SEQ ID NO:80;

fifteen predicted casein kinase II phosphorylation sites (PS00006) located at about amino acid residues 28 to 31, 108 to 111, 152 to 155, 169 to 172, 180 to 183, 209 to 212, 246 to 249, 263 to 266, 335 to 338, 374 to 37, 476 to 479, 608 to 611, 629 to 632, 636 to 639, and 735 to 738 of SEQ ID NO:80;

three predicted tyrosine kinase phosphorylation sites (PS00007) located at about amino acid residues 147 to 154, 705 to 713, and 716 to 722 of SEQ ID NO:80;

one predicted RGD cell attachment sequence (PS00016) located about amino acid residues 181 to 183 of SEQ ID NO:80;

one predicted glycosaminoglycan attachment site (PS00002) located at about amino acid residues 581 to 584 of SEQ ID NO:80;

seven predicted N-glycosylation sites (PS00001) located at about amino acid residues 261 to 264, 420 to 423, 485 to 488, 546 to 549, 570 to 573, 676 to 679, and 795 to 798 of SEQ ID NO:80; and

seven predicted N-myristylation sites (PS00008) located at about amino acid residues 36 to 41, 79 to 84, 184 to 189, 483 to 488, 538 to 543, 686 to 691, and 777 to 782 of SEQ ID NO:80.

For general information regarding PFAM identifiers, PS prefix and PF prefix domain identification numbers, refer to Sonnhammer et al. (1997) Protein 28:405-420 and http://www.psc.edu/general/software/packages/pfam/pfam.html.

A plasmid containing the nucleotide sequence encoding human 57779 was deposited with American Type Culture Collection (ATCC), 10801 University Boulevard, Manassas, Va. 20110-2209, on ______ and assigned Accession Number ______. This deposit will be maintained under the terms of the Budapest Treaty on the International Recognition of the Deposit of Microorganisms for the Purposes of Patent Procedure. This deposit was made merely as a convenience for those of skill in the art and is not an admission that a deposit is required under 35 U.S.C. §112.

| | | | | ATCC |
|---|---|---|---|---|
| Gene Name | cDNA | Protein | Coding Region | accession number |
| 23566 | SEQ ID NO: 73 | SEQ ID NO: 74 | SEQ ID NO: 75 | |
| 33489 | SEQ ID NO: 76 | SEQ ID NO: 77 | SEQ ID NO: 78 | |
| 57779 | SEQ ID NO: 79 | SEQ ID NO: 80 | SEQ ID NO: 81 | |

23566 Protein

The 23566 protein contains a significant number of structural characteristics in common with members of the carboxypeptidase family, in particular the zinc carboxypeptidase family, of proteins. The term “family” when referring to the protein and nucleic acid molecules of the invention means two or more proteins or nucleic acid molecules having a common structural domain or motif and having sufficient amino acid or nucleotide sequence homology as defined herein. Such family members can be naturally or non-naturally occurring and can be from either the same or different species. For example, a family can contain a first protein of human origin as well as other distinct proteins of human origin, or alternatively, can contain homologues of non-human origin, e.g., rat or mouse proteins. Members of a family can also have common functional characteristics.

Zinc carboxypeptidases are structurally and functionally related. They share a characteristic fold that includes a zinc carboxypeptidase zinc binding motif, which can be have one of two possible sequences: [PK]-X-[LIVMFY]-X-[LIVMFY]-X-X-X-X-H-[STAG]-X-E-X-[LIVM]-[STAG]-X-X-X-X-X-X-[LIVMFYTA], wherein X represents any amino acid and the H (hisidine) and E (glutamic acid) residues are directly involved in zinc ion coordination and substrate catalysis, respectively; or H-[STAG]-X-X-X-[LIVME]-X-X-[LIVMFYW]-P-[FYW], wherein X represents any amino acid and the H (histidine) residue is directly involved in zinc ion cooridination. A zinc carboxypeptidase zinc-binding region 1 signature motif includes the former sequence. Zinc carboxypeptidases belong to the families M14A/Ml4B in the classification of peptidases.

A 23566 polypeptide can include a “zinc carboxypeptidase domain” or regions homologous with a “zinc carboxypeptidase domain”.

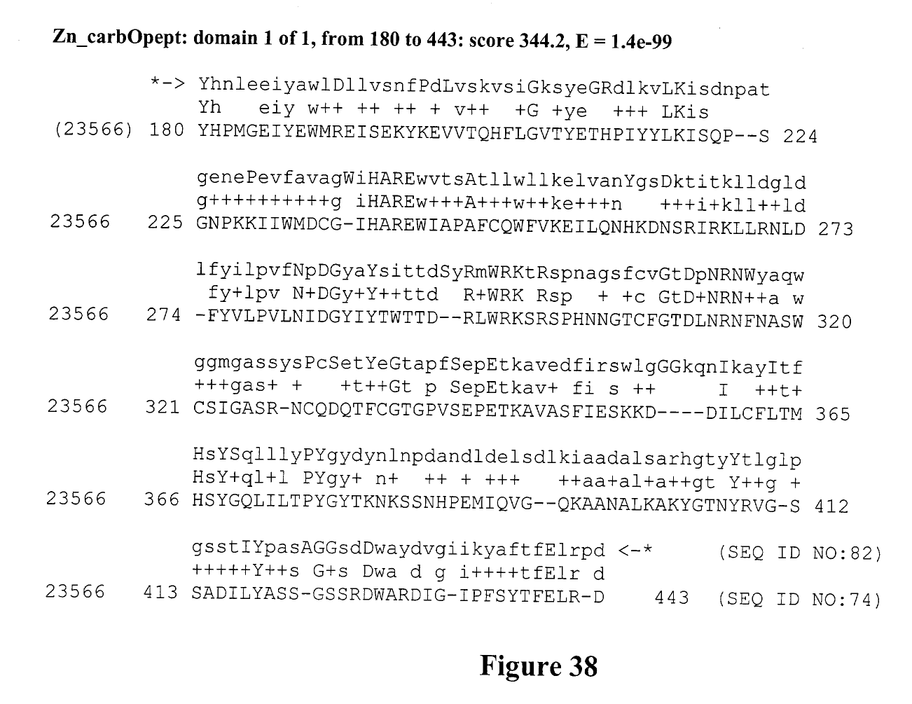

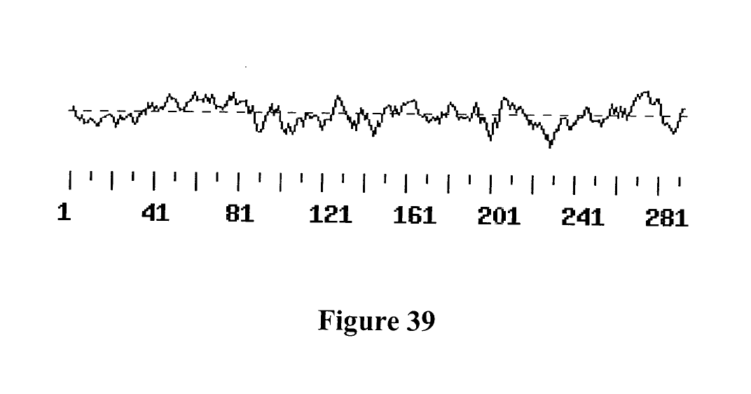

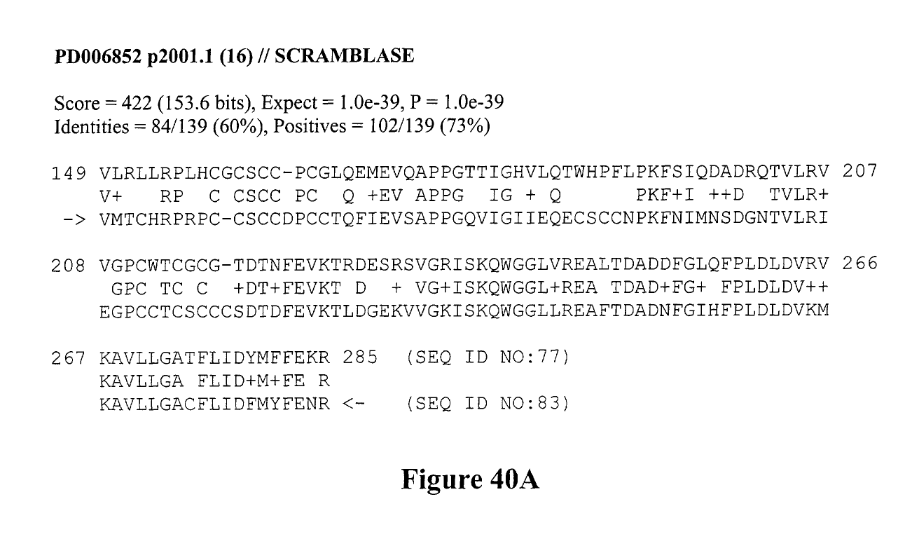

As used herein, the term “zinc carboxypeptidase domain” includes an amino acid sequence of about 100 to 400 amino acid residues in length and having a bit score for the alignment of the sequence to the zinc carboxypeptidase domain (HMM) of at least 200. Preferably, a zinc carboxypeptidase domain includes at least about 200 to 300 amino acids, more preferably about 250 to 275 amino acid residues, or about 260 to 265 amino acids and has a bit score for the alignment of the sequence to the zinc carboxypeptidase domain (HMM) of at least 250, 300 or greater. The zinc carboxypeptidase domain (HMM) has been assigned the PFAM Accession Number PF00246 (http;//genome.wustl.edu/Pfam/.html). An alignment of the zinc carboxypeptidase domain (amino acids 180 to 443 of SEQ ID NO:74) of human 23566 with a consensus amino acid sequence derived from a hidden Markov model is depicted in FIG. 38.

In a preferred embodiment 23566 polypeptide or protein has a “zinc carboxypeptidase domain” or a region which includes at least about 200 to 300 more preferably about 250 to 275 or 260 to 265 amino acid residues and has at least about 60%, 70% 80% 90% 95%, 99%, or 100% homology with a “zinc carboxypeptidase domain,” e.g., the zinc carboxypeptidase domain of human 23566 (e.g., residues 180 to 443 of SEQ ID NO:74).

To identify the presence of a “zinc carboxypeptidase” domain in a 23566 protein sequence, and make the determination that a polypeptide or protein of interest has a particular profile, the amino acid sequence of the protein can be searched against the PFAM database of HMMs (e.g., the PFAM database, release 2.1) using the default parameters (http://www.sanger.ac.uk/Software/Pfam/HMM search). For example, the hmmsf program, which is available as part of the HMMER package of search programs, is a family specific default program for MILPAT0063 and a score of 15 is the default threshold score for determining a hit. Alternatively, the threshold score for determining a hit can be lowered (e.g., to 8 bits). A description of the PFAM database can be found in Sonhammer et al. (1997) Proteins 28(3):405-420 and a detailed description of HMMs can be found, for example, in Gribskov et al. (1990) Meth. Enzymol. 183:146-159; Gribskov et al. (1987) Proc. Natl. Acad. Sci. USA 84:4355-4358; Krogh et al. (1994) J. Mol. Biol. 235:1501-1531; and Stultz et al. (1993) Protein Sci. 2:305-314, the contents of which are incorporated herein by reference. A search was performed against the HMM database resulting in the identification of a “zinc carboxypeptidase domain” in the amino acid sequence of human 23566 at about residues 180 to 443 of SEQ ID NO:74 (see FIG. 38).

In some embodiments, a 23566 molecule includes at least one zinc carboxypeptidase zinc-binding region 1 signature motif. As used herein, a “zinc carboxypeptidase zinc-binding region 1 signature motif” includes a sequence of about 15 to 30 amino acid residues defined by the sequence: [PK]-X-[LIVMFY]-X-[LIVMFY]-X-X-X-X-H-[STAG]-X-E-X-[LIVM]-[STAG]-X-X-X-X-X-X-[LIVMFYTA], wherein X represents any amino acid. A zinc carboxypeptidase zinc-binding region 1 signature motif, as defined, can be involved in the cooridination of a zinc ion and the hydrolysis of a peptide bond, e.g., a carboxyterminal peptide bond. More preferably, a zinc carboxypeptidase zinc-binding region 1 signature motif includes about 20 to 25 amino acid residues, and most preferably about 23 amino acid residues. The zinc carboxypeptidase zinc-binding region 1 signature motif has been assigned the Prosite Accession Number PS00132. Human 23566 contains a zinc carboxypeptidase zinc-binding region 1 signature motif located at about amino acid residues 229 to 251 of SEQ ID NO:74.

In preferred embodiments, a 23566 polypeptide or protein has at least one zinc carboxypeptidase zinc-binding region 1 singature motif, or a region which includes at least 15, 20, or preferably 23 amino acid residues and has at least 70%, 80%, 90%, 95%, or 100% homology with a “zinc carboxypeptidase zinc-binding region 1 singnature motif”, e.g., the zinc carboxypeptidase zinc-binding region 1 signature motif of human 23566 (e.g., residues 229 to 251 of SEQ ID NO:74).

In some embodiments, the 23566 molecule can further include a signal sequence. As used herein, a “signal sequence” refers to a peptide of about 40-80 amino acid residues in length which occurs at the N-terminus of secretory and integral membrane proteins and which contains a majority of hydrophobic amino acid residues. For example, a signal sequence contains at least about 50-70 amino acid residues, preferably about 68 amino acid residues, and has at least about 40-70%, preferably about 50-65%, and more preferably about 55-60% hydrophobic amino acid residues (e.g., alanine, valine, leucine, isoleucine, phenylalanine, tyrosine, tryptophan, or proline). Such a “signal sequence”, also referred to in the art as a “signal peptide”, serves to direct a protein containing such a sequence to a lipid bilayer where the protein is passed through the lipid bilayer, e.g., for the purpose of sorting and secretion, or inserted into the lipid bilayer. A predicted signal sequence was identified in human 23566, located at about amino acid residues 1-68 of SEQ ID NO:74. The “signal sequence” can be cleaved during processing resulting in the fomation, e.g., of a mature protein. The mature human 23566 protein is predicted to correspond to about amino acid residues 69 to 444 of SEQ ID NO:74.

In preferred embodiments, a 23566 protein includes a signal sequence, or a region which includes at least 30, 40, 50, 60, 65, or more amino acid residues and shares at least 70%, 80%, 90%, 95%, 98%, or more homology with a “signal sequenc”, e.g., the signal sequence of human 23566 (e.g., about amino acid residues 1 to 68 of SEQ ID NO:74).

A 23566 family member can include at least one zinc carboxypeptidase domain. Furthermore, a 23566 family member can include at least one zinc carboxypeptidase zinc-binding region 1 signature motif; one at least one singal sequence; at least one, two, three, four, preferably five predicted protein kinase C phosphorylation sites (PS00005); at least one, two, three, four, and preferably five predicted casein kinase II phosphorylation sites (PS00006); and at least one, two, three, and preferably four predicted N-glycosylation sites (PS00001); at least one predicted Tyrosine kinase phosphorylation site (PS00007); at least one, two, three, four, preferably five 5 predicted N-myristylation sites (PS00008); and at least one predicted Amidation site (PS00009).

As the 23566 polypeptides of the invention may modulate 23566-mediated activities, they may be useful as of for developing novel diagnostic and therapeutic agents for 23566-mediated or related disorders, as described below.

As used herein, a “23566 activity”, “biological activity of 23566” or “functional activity of 23566”, refers to an activity exerted by a 23566 protein, polypeptide or nucleic acid molecule. For example, a 23566 activity can be an activity exerted by 23566 in a physiological milieu on, e.g., a 23566-responsive cell or on a 23566 substrate, e.g., a protein substrate. A 23566 activity can be determined in vivo or in vitro. In one embodiment, a 23566 activity is a direct activity, such as an association with a 23566 target molecule. A “target molecule” or “binding partner” is a molecule with which a 23566 protein binds or interacts in nature. In some embodiments, a 23566 protein is a receptor, e.g., for a polypeptide ligand. In an exemplary embodiment, 23566 is an enzyme that binds to polypeptide substrates and hydrolyzes polypeptide bonds, e.g., polypeptide bonds present at the carboxy-terminus of the polypeptide substrate.

A 23566 activity can also be an indirect activity, e.g., a cellular signaling activity mediated by interaction of the 23566 protein with a 23566 ligand or receptor. The features of the 23566 molecules of the present invention can provide similar biological activities as zinc carboxypeptidase family members. For example, the 23566 proteins of the present invention can have one or more of the following activities: (1) formation of a zinc ion complex with a carbonyl group of a substrate polypeptide and polarization of the carbon-oxygen bond; (2) formation of a tetrahedral intermediate due to attack of the carbonyl carbon by water in a reaction assisted by a carboxylate side chain of glutamate; (3) production of a dianion intermediate by rapid ionization of the tetrahedral intermediate produced; (4) cleavage of the C—N bond of the substrate to collapse the tetrahedral intermediate; (5) binding the carboxy-terminus of polypeptides; (6) hydrolyzing polypeptides to remove/release a carboxy-terminal residue; (7) participating in digestion of polypeptides/proteins; (8) processing prohormones; (9) regulating peptide hormones (e.g., prior to or after interaction with plasma membrane receptors); (10) inactivating potent vasoactive and inflammatory peptides (e.g., kinins or anaphylatoxins) released into the circulation; or (11) inactivating potent neuroactive peptides (e.g., bombesin, bradykinin, neuropeptide substance P).

In addition, the 23566 molecules of the invention can be expected to function in tissues in which they are expressed. For example, human 23566 is expressed in the brain (e.g., the cortex and hypothalamus), arteries, skeletal muscle, kidney, lymph nodes, and pancreas, as well as a variety of other tissues (see Example 11). Thus, the 23566 molecules can act as novel diagnostic targets and therapeutic agents for controlling disorders, such as conditions involving aberrant or deficient digestion (e.g., disorders of the small intestine), disorders of the pancreas, inflammatory diseases, neurological disorders and/or hormonal disorders/imbalances.

33489 Protein

The 33489 protein contains a significant number of structural characteristics in common with members of the scramblase family. The term “family” when referring to the protein and nucleic acid molecules of the invention means two or more proteins or nucleic acid molecules having a common structural domain or motif and having sufficient amino acid or nucleotide sequence homology as defined herein. Such family members can be naturally or non-naturally occurring and can be from either the same or different species. For example, a family can contain a first protein of human origin as well as other distinct proteins of human origin, or alternatively, can contain homologues of non-human origin, e.g., rat or mouse proteins. Members of a family can also have common functional characteristics.

Scramblase family members are, typically, Type II transmembrane proteins with an intracellular amino terminal domain, a transmembrane span, and a small extracellular carboxy terminal domain. Zhou et al. (1998), Biochemistry 37:2356, have identified key conserved residues important for scramblase catalytic activity. These residues include aspartic acids 273, 275, and 284, phenylalanine 277 and 281, and isoleucine 279 of TrEMBL Entry 015162.

Included among these conserved residues, are residues that can sense the binding of a Ca²⁺ ligand.

A 33489 polypeptide can include a “scramblase domain” or regions homologous with a “scramblase domain”.

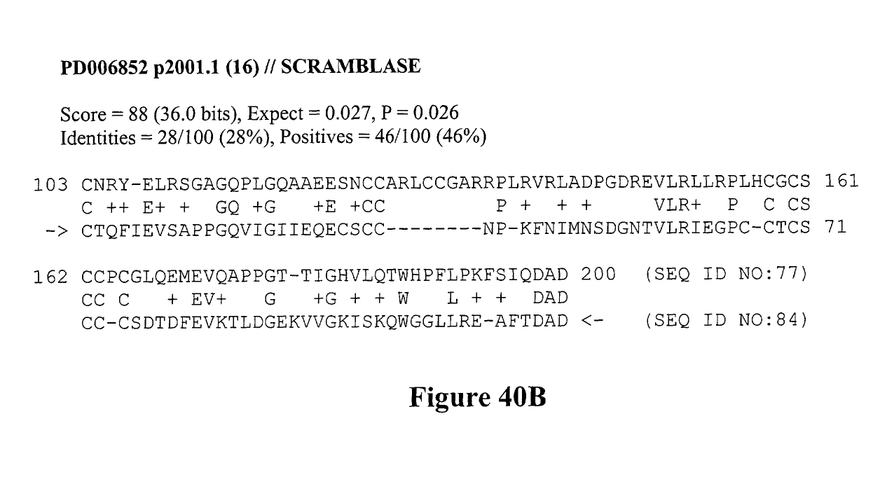

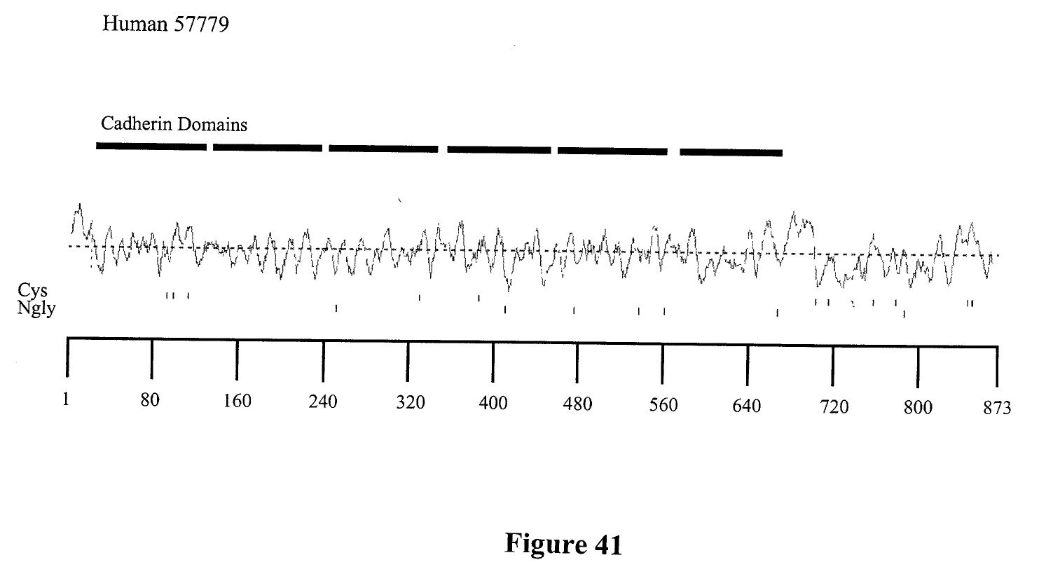

As used herein, the term “scramblase domain” includes an amino acid sequence of about 100 to 300 amino acid residues in length having a BLAST score for the alignment of the sequence to the scramblase domain of at least 160, or a bit score for the alignment of at least 70. Preferably, a scramblase domain includes at least about 140 to 220 amino acids, more preferably about 160 to 200 amino acid residues, or about 170 to 185 amino acids and has a score for the alignment of the sequence to the scramblase domain of at least 200, 250, 300, 350, 360, 370 or greater. The scramblase domain is homologous to ProDom family PD006852 (ProDomain Release 2000.1; www.toulouse.inra.fr/prodom.html; see also ProDomain No. 5246, Release 1999.2) An alignment of a portion of the scramblase domain (amino acid residues 149 to 285 of SEQ ID NO:77) of human 33489 with a consensus amino acid sequence (SEQ ID NO:83) derived from the ProDom family PD006852 is depicted in FIG. 40A, while an alignment of another portion of the scramblase domain (amino acid residues 103 to 200 of SEQ ID NO:77) of human 23566 with a consensus amino acid sequence (SEQ ID NO:84) derived from the ProDom family PD006852 is depicted in FIG. 40B.

In a preferred embodiment, a 33489 polypeptide or protein has a “scramblase domain” or a region which includes at least about 140 to 220 more preferably about 160 to 200 or 170 to 185 amino acid residues and has at least about 50%, 60%, 70% 80% 90% 95%, 99%, or 100% homology with a “scramblase domain,” e.g., the scramblase domain of human 33489 (e.g., residues 103 to 285 of SEQ ID NO:77). The 33489 polypeptide preferably includes at least one, preferably two and most preferably three conserved aspartic acid residues, e.g., located at about amino acids 250, 252, and 261 of SEQ ID NO:77, and conserved phenylalanine residues, e.g., residues located at about amino acids 254 and 258 of SEQ ID NO:77. Preferably, the conserved amino acid side chains participate in sensing the binding of Ca²⁺ ligand.

To identify the presence of a “scramblase” domain in a 33489 protein sequence, and make the determination that a polypeptide or protein of interest has a particular profile, the amino acid sequence of the protein can be searched against a database of domains, e.g., the ProDom database (Corpet et al. (1999), Nucl. Acids Res. 27:263-267). The ProDom protein domain database consists of an automatic compilation of homologous domains. Current versions of ProDom are built using recursive PSI-BLAST searches (Altschul SF et al. (1997) Nucleic Acids Res. 25:3389-3402; Gouzy et al. (1999) Computers and Chemistry 23:333-340) of the SWISS-PROT 38 and TREMBL protein databases. The database automatically generates a consensus sequence for each domain. A BLAST search was performed against the ProDom database resulting in the identification of a “scramblase” domain in the amino acid sequence of human 33489 at about residues 103 to 285 of SEQ ID NO:77 (see FIGS. 40A-B).

A 33489 family member preferably has a Type II transmembrane topology, e.g., an N-terminal cytoplasmic domain, a transmembrane domain, and a short C-terminal extracellular domain.

As used with respect to 33489 family members, an “N-terminal cytoplasmic domain” refers to a region of the polypeptide located in the interior of the cell, and can include an amino acid sequence having about 230 to 300 amino acids, preferably about 250 to 280 amino acids, and more preferably about 255 to 270 amino acids. The C-terminal amino acid residue of an “N-terminal cytoplasmic domain” is adjacent to an N-terminal amino acid residue of a transmembrane domain in a naturally-occurring 33489 or 33489-like protein, e.g., about residues 266 to 282 of SEQ ID NO:77. For example, an N-terminal cytoplasmic domain is located at about amino acid residues 1 to 265 of SEQ ID NO:77.

In preferred embodiments, a 33489 polypeptide or protein has an N-terminal cytoplasmic domain, or a region which includes at least 200, 230, 250, 255 or more amino acid residues and has at least 70%, 80%, 90%, 95%, 98%, 99%, or more homology with an “N-terminal cytoplasmic domain” of human 33489, e.g., about amino acid residues 1 to 265 or SEQ ID NO:77.

As used herein, a “transmembrane domain” includes an amino acid sequence of about 15 amino acid residues in length which spans the plasma membrane. A transmembrane domain can include about at least 17, 20, 24, 25, or 30 amino acid residues and span the plasma membrane. Transmembrane domains are rich in hydrophobic residues, and typically have an α-helical structure. In a preferred embodiment, at least 50%, 60%, 70%, 80%, 90%, 95% or more of the amino acids of a transmembrane domain are hydrophobic, e.g., leucines, isoleucines, tyrosines, or tryptophans. Transmembrane domains are described in, for example, htto://pfam.wustl.edu/cgi-bin/getdesc?name=7tm-1, and Zagotta W. N. et al., (1996) Annual Rev. Neuronsci. 19: 235-63, the contents of which are incorporated herein by reference. A transmembrane domain was detected in human 33489, located at about amino acid residues 266 to 282 of SEQ ID NO:77.

In preferred embodiments, a 33489 polypeptide or protein has at least one transmembrane domain, or a region which includes at least 17 or more amino acid residues and has at least 70%, 80%, 90%, or 100% homology with a “transmembrane domain”, e.g., a transmembrane domain of human 33489, e.g., about amino acid residues 266 to 282 of SEQ ID NO:77.

When located at the C-terminal domain, an extracellular domain is referred to herein as a “C-terminal extracellular domain”, or as a “C-terminal extracellular loop” in the amino acid sequence of the protein. As used herein with respect to 33489 family members, a “C-terminal extracellular domain” includes an amino acid sequence having about 1-20, preferably about 1-15, more preferably about 5-15, more preferably about 10-15, even more preferably about 12 amino acid residues in length and is located outside of a cell or extracellularly. The N-terminal amino acid residue of a “C-terminal extracellular domain” is adjacent to a C-terminal amino acid residue of a transmembrane domain in a naturally-occurring 33489 or 33489-like protein. For example, a C-terminal extracellular domain is located at about amino acid residues 283 to 295 of SEQ ID NO:77.

In preferred embodiments, a 33489 polypeptide or protein has a C-terminal extracellular domain, or a region which includes at least 1, 5, 10, or even 12 or more amino acid residues and has at least 70%, 80%, 90%, or 100% homology with a “C-terminal extracellular domain” of human 33489, e.g., about amino acid residues 283 to 295 of SEQ ID NO:77.

A 33489 family member can include at least one scramblase domain. In addition, a 33489 family member can have type II membrane topology and include an N-terminal cytoplasmic domain, a transmembrane domain, and a short C-terminal extracellular domain. Furthermore, a 33489 family member can include at least one, two, three, four, or preferably five predicted casein kinase II phosphorylation sites (PS00006); at least one predicted glycosaminoglycan attachment site (PS00002); at least one a proline rich potential SH3-domain binding site; and at least one, two, three, four, or preferably five predicted N-myristylation sites (PS00008).

As the 33489 polypeptides of the invention may modulate 33489-mediated activities, they may be useful as of for developing novel diagnostic and therapeutic agents for 33489-mediated or related disorders, as described below.

As used herein, a “33489 activity”, “biological activity of 33489” or “functional activity of 33489”, refers to an activity exerted by a 33489 protein, polypeptide or nucleic acid molecule. For example, a 33489 activity can be an activity exerted by 33489 in a physiological milieu on, e.g., a 33489-responsive cell or on a 33489 substrate, e.g., a protein substrate. A 33489 activity can be determined in vivo or in vitro. In one embodiment, a 33489 activity is a direct activity, such as an association with a 33489 target molecule. A “target molecule” or “binding partner” is a molecule with which a 33489 protein binds or interacts in nature. In an exemplary embodiment, 33489 is a scramblase enzyme. As used herein, a “scramblase” activity refers to a catalytic activity which accelerates the movement of a membrane lipid, e.g., a phospholipid, e.g., an amino phospholipid, from one leaflet of the membrane bilayer to the other.

A 33489 activity can also be an indirect activity, e.g., a cellular signaling activity mediated by the redistribution of a membrane lipid from the inner leaflet to the outer leaflet of the plasma membrane, and vice versa. Based on the above-described sequence similarities, the 33489 molecules of the present invention are predicted to have similar biological activities as scramblase family members. For example, the 33489 proteins of the present invention can have one or more of the following activities: (1) equilibration of membrane lipid molecules between membrane leaflets, e.g., of amino phospholipids, e.g., phosphoserine, phosphocholine, etc.; (2) presentation of inner membrane leaflet phospholipids on the outer membrane leaflet, e.g., in response to a cell signal, e.g., a Ca²⁺ signal; (3) modulation of membrane fluidity, thereby altering fusion efficiency of the cell with another cell or with a retrovirus; (4) activation of an extracellular protease cascade, e.g., a coagulation system, or a complement system; (5) activation of an apoptotic process; and (6) stimulation of a noninflammatory engulfinent process.

In addition, the 33489 molecules of the invention can be expected to function in tissues in which they are expressed. For example, human 33489 is expressed in the brain (e.g., the cortex and hypothalamus), arteries, skeletal muscle, kidney, lymph nodes, and pancreas, as well as a variety of other tissues (see Example 11). Thus, the 33489 molecules can act as novel diagnostic targets and therapeutic agents for controlling a variety of disorders, including but not limited to cardiovascular disorders (e.g., disorders of the heart and/or blood vessels), apoptotic disorders, red blood cell disorders (e.g., blood clotting disorders, anemias), inflammatory disorders (e.g., complement activity disorders), viral disorders, cell engulfinent disorders, or cellular proliferation and/or differentiation disorders.

57779 Protein

The 57779 protein contains a significant number of structural characteristics in common with members of the protocadherin family including a signal peptide, multiple, e.g., six, cadherin ectodomains, and two transmembrane domains. The term “family” when referring to the protein and nucleic acid molecules of the invention means two or more proteins or nucleic acid molecules having a common structural domain or motif and having sufficient amino acid or nucleotide sequence homology as defined herein. Such family members can be naturally or non-naturally occurring and can be from either the same or different species. For example, a family can contain a first protein of human origin as well as other distinct proteins of human origin, or alternatively, can contain homologues of non-human origin, e.g., rat or mouse proteins. Members of a family can also have common functional characteristics.

The protocadherin family of proteins is characterized by a common domain organization. A protocadherin can contain a signal peptide, typically, without a preceding prosegment, six or more cadherin repeats in its ectodomain, at least one transmembrane domain, and at least one intracellular domain (see, e.g., Wu and Maniatis (2000) Proc. Natl. Acad. Sci. USA 97:3124, and Suzuki (1996) J. Cell Sci. 109:2609)

A 57779 polypeptide can include a “cadherin domain” or regions homologous with a “cadherin domain.” The canonical cadherin domain is an extracellular protein module, frequently repeated in cell surface membrane proteins. The domain folds as a predominantly β-barrel of seven β-strands, and one short α-helix (Shapiro et al. (1995), Nature 374:327-337). Cadherin domains from opposing cells can dimerize. This physical association structurally rigidifies and extends the folded structure (Nagar et al. (1996) Nature 380:360-364). The calcium ion which is essential to cadherin functionality can bind to residues on the short alpha helix, and to loops between the β-strands on one face of the molecule (Overduin et al. (1995) Science 267:386-389).

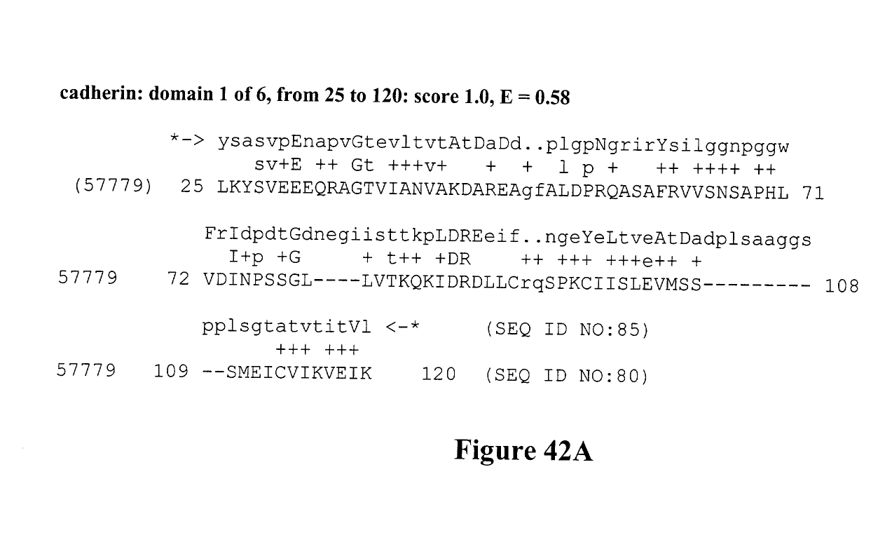

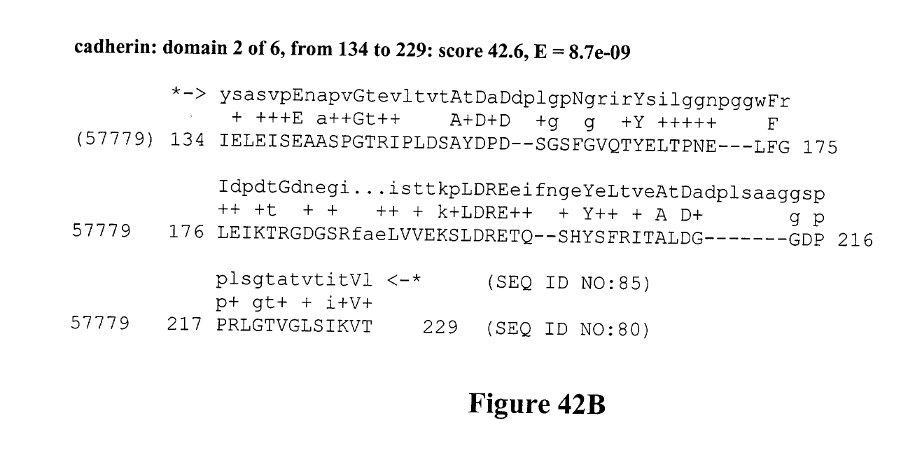

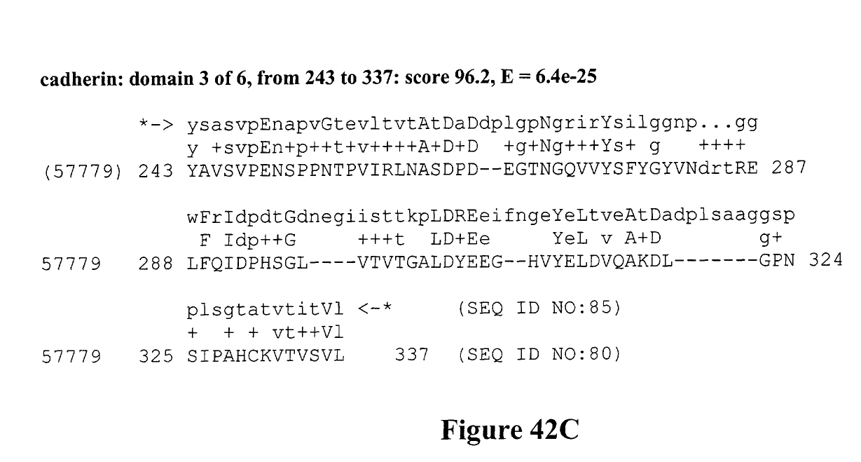

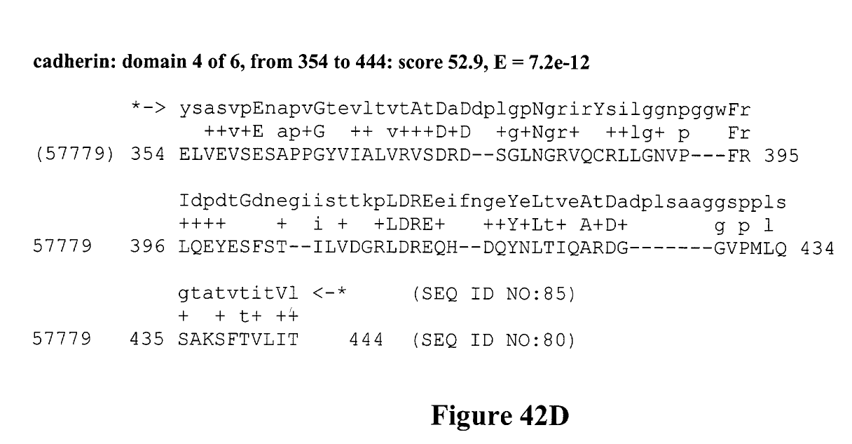

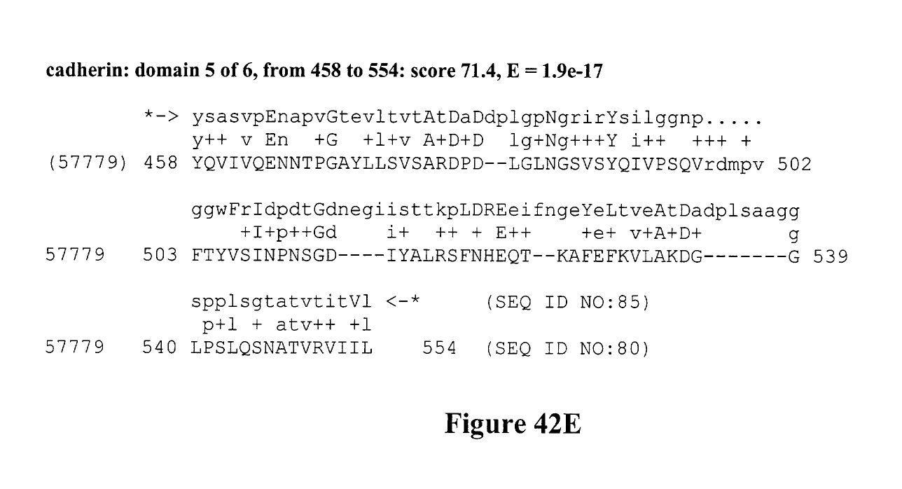

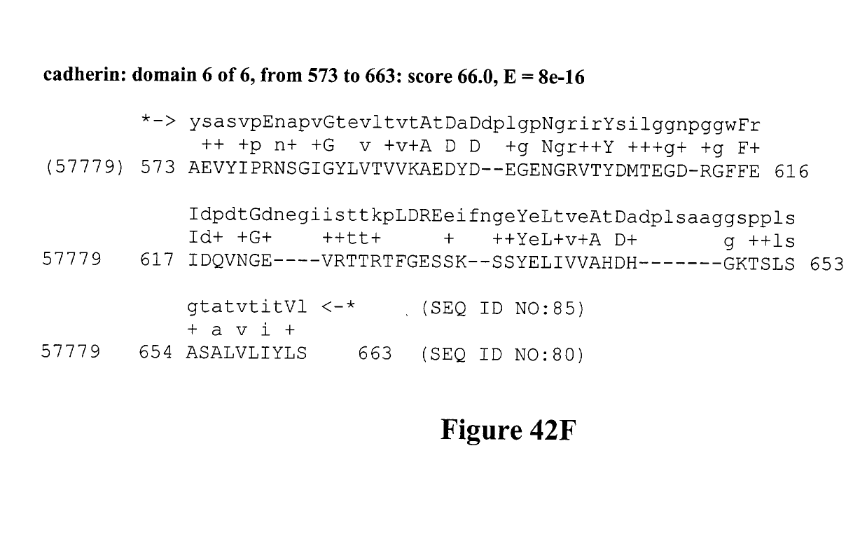

As used herein, the term “cadherin domain” includes an amino acid sequence of about 70 to 150 amino acid residues in length and having a bit score for the alignment of the sequence to the cadherin domain (HMM) of at least 20. Preferably, a cadherin domain includes at least about 70 to 150, 80 to 140, preferably about 90 to 130, or more preferably about 100 to 120 amino acids and has a bit score for the alignment of the sequence to the cadherin domain (HMM) of at least 20, 30, 40 or greater. In cases in which multiple cadherin domains are repeated, e.g., six times, the combined bit score for the alignment of the sequence of the cadherin domain (HMM) is preferably at least 250, 270, 290, 310 or greater. The cadherin domain (HMM) has been assigned the PFAM Accession Number PF00028 (at the online resouce for PFAM available from Washington University, St. Louis Mo., USA). Alignments of the cadherin domains (amino acids 25 to 120, 134 to 229, 243 to 337, 354 to 444, 458 to 554, and 573 to 663 of SEQ ID NO:80) of human 57779 with a consensus amino acid sequence (SEQ ID NO:85) derived from a hidden Markov model in the PFAM database are depicted in FIGS. 42A-F.

In a preferred embodiment, a 57779 polypeptide or protein has at least one, two, three, four, five, and preferably six “cadherin domains” or regions which includes at least 70 to 150, 80 to 140, preferably about 90 to 130, or more preferably about 100 to 120 amino acids residues and have at least about 50%, 60%, 70% 80% 90% 95%, 99%, or 100% homology with a “cadherin domain,” e.g., one of the cadherin domains of human 57779 (e.g., residues 25 to 120, 134 to 229, 243 to 337, 354 to 444, 458 to 554, and 573 to 663 of SEQ ID NO:80).

To identify the presence of a “cadherin” domain in a 57779 protein sequence, and make the determination that a polypeptide or protein of interest has a particular profile, the amino acid sequence of the protein can be searched against the PFAM database of HMMs (e.g., the PFAM database, release 2.1) using the default parameters (http://www.sanger.ac.uk/Software/Pfam/HMM_search). For example, the hmmsf program, which is available as part of the HMMER package of search programs, is a family specific default program for MILPAT0063 and a score of 15 is the default threshold score for determining a hit. Alternatively, the threshold score for determining a hit can be lowered (e.g., to 8 bits). A description of the PFAM database can be found in Sonhammer et al. (1997) Proteins 28(3):405-420 and a detailed description of HMMs can be found, for example, in Gribskov et al. (1990) Meth. Enzymol. 183:146-159; Gribskov et al. (1987) Proc. Natl. Acad. Sci. USA 84:4355-4358; Krogh et al. (1994) J. Mol. Biol. 235:1501-1531; and Stultz et al. (1993) Protein Sci. 2:305-314, the contents of which are incorporated herein by reference. A search was performed against the HMM database resulting in the identification of six “cadherin domains” in the amino acid sequence of human 57779, located at about amino acid residues 25 to 120, 134 to 229, 243 to 337, 354 to 444, 458 to 554, and 573 to 663 of SEQ ID NO:80. The results of the search are depicted by the alignments shown in FIGS. 42A-B.

Similarly, the presence of multiple “cadherin” domains in a 57779 protein sequence was further evidenced by a search against a SMART database (Simple Modular Architecture Research Tool, available online from the EMBL at Heidelberg Germany) of HMMs as described in Schultz et al. (1998), Proc. Natl. Acad. Sci. USA 95:5857 and Schultz et al. (200) Nucl. Acids Res 28:231. The database contains domains identified by profiling with the hidden Markov models of the HMMer2 search program (R. Durbin et al. (1998) Biological sequence analysis: probabilistic models of proteins and nucleic acids. Cambridge University Press.; also available online from the HMMER site at Washington University, St. Louis Mo., USA). The database also is extensively annotated and monitored by experts to enhance accuracy. A search was performed against the SMART HMM database resulting in the identification of a “cadherin” domain in the amino acid sequence of human 57779 at about residues 25 to 120, 134 to 229, 243 to 337, 354 to 444, 458 to 554, and 573 to 663 of SEQ ID NO:80.

In some embodiments, a 57779 protein includes a cadherin extracellular domain signature motif. As used herein, a “cadherin extracellular domain signature motif” includes a sequence of at least nine amino acids having the consensus amino acid sequence: [LIV]-X-[LIV]-X-D-X-N-D-[NH]-X-P (SEQ ID NO:86), wherein X represents any amino acid. More preferably, a cadherin extracellular domain signature motif includes a sequence of at least eleven amino acid residues. The cadherin extracellular domain signature motif has been given the Prosite accession number PS00232. Human 57779 contains five identical matches to the cadherin extracellular domain signature motif, located at about amino acid residues 117 to 127, 226 to 236, 334 to 344, 441 to 451, and 551 to 561 of SEQ ID NO:80.

In a preferred embodiment, a 57779 protein includes at least one, two, three, four, and preferably five cadherin extracellular domain signature motifs, or sequences including at least nine, preferably eleven amino acid residues and having at least 70%, 80%, 90%, or even 100% homology with a “cadherin extracellular domain signature motif”, e.g., at least one cadherin extracellular domain signature motif of human 57779, e.g., about amino acid residues 117 to 127, 226 to 236, 334 to 344, 441 to 451, and 551 to 561 of SEQ ID NO:80.

In one embodiment, a 57779 protein includes at least one and preferably two extracellular domains. When located at the N-terminal end of the protein, the extracellular domain is referred to herein as an “N-terminal extracellular domain”, or as an “N-terminal extracellular loop” in the amino acid sequence of the protein. As used herein in reference to a 57779 protein, an “N-terminal extracellular domain” includes an amino acid sequence having about 1-900, preferably about 400-800, more preferably about 500-700, more preferably about 600-680, even more preferably about 650 amino acid residues in length and is located outside of a cell or extracellularly. The C-terminal amino acid residue of a “N-terminal extracellular domain” is adjacent to an N-terminal amino acid residue of a transmembrane domain in a naturally-occurring 57779 or 57779-like protein. For example, an N-terminal extracellular domain is located at about amino acid residues 1-676 of SEQ ID NO:80 (and 26-676 of SEQ ID NO:80 of the mature protein). Preferably, the N-terminal extracellular domain is capable of interacting (e.g., binding to) with an extracellular signal, for example, a cell surface molecule (e.g., a cadherin molecule). Most preferably, the N-terminal extracellular domain mediates protein-protein interactions, signal transduction and/or cell adhesion or aggregation.

In one embodiment, the N-terminal extracellular domain contains at least one, two, three, four, five, and preferably six cadherin domains, e.g., a cadherin domain as described herein. Preferably, the cadherin domains in the extracellular domain of a 57779 protein mediate protein-protein interactions, signal transduction and/or cell adhesion or aggregation. Preferably, the N-terminal extracellular domain is capable of interacting (e.g., binding to) with an extracellular molecule, for example, a cell surface molecule (e.g., a cadherin molecule).

In preferred embodiments, a 57779 polypeptide or protein has an N-terminal extracellular domain, or a region which includes at least 400, 500, 600, 650, or more amino acid residues and shares at least 70%, 80%, 90%, 95%, 98%, 99%, or more homology with an “N-terminal extracellular domain” of human 57779, e.g., about amino acid residues Ito 676 of SEQ ID NO:80.

When located at the C-terminal end of a protein an extracellular domain is referred to herein as a “C-terminal extracellular domain”, or as a “C-terminal extracellular loop” in the amino acid sequence of the protein. As used herein in reference to 57779 proteins, a “C-terminal extracellular domain” includes an amino acid sequence having about 1-20, preferably about 1-15, more preferably about 5-15, more preferably about 10-15, even more preferably about 13 amino acid residues in length and is located outside of a cell or extracellularly. The N-terminal amino acid residue of a “C-terminal extracellular domain” is adjacent to a C-terminal amino acid residue of a transmembrane domain in a naturally-occurring 57779 or 57779-like protein. For example, a C-terminal extracellular domain of human 57779 is located at about amino acid residues 860-873 of SEQ ID NO:80.

In preferred embodiments, a 57779 polypeptide or protein has a C-terminal extracellular domain, or a region which includes at least 1, 5, 10, 13, or more amino acid residues and shares at least 70%, 80%, 90%, or 100% homology with a “C-terminal extracellular domain” of human 57779, e.g., about amino acid residues 860 to 873 of SEQ ID NO:80.

In one embodiment, the 57779 proteins of the present invention contain at least one RGD cell attachment site. As used herein, the term “RGD cell attachment site” refers to a cell adhesion sequence consisting of amino acids Arg-Gly-Asp typically found in extracellular matrix proteins such as collagens, laminin and fibronectin, among others (reviewed in Ruoslahti, E. (1996) Annu. Rev. Cell Dev. Biol. 12:697-715). Preferably, the RGD cell attachment site is located in the extracellular domain of a 57779 protein and interacts (e.g., binds to) a cell surface receptor, such as an integrin receptor. As used herein, the term “integrin” refers to a family of receptors comprising αβ heterodimers that mediate cell attachment to extracellular matrices and cell-cell adhesion events. The α subunits vary in size between 120 and 180 kd and are each noncovalently associated with a β subunit (90-110 kd) (reviewed by Hynes (1992) Cell 69:11-25). Most integrins are expressed in a wide variety of cells, and most cells express several integrins. There are at least 8 known β subunits and 14 known α subunits. The majority of the integrin ligands are extracellular matrix proteins involved in substratum cell adhesion such as collagens, laminin, fibronectin among others. An RGD cell attachment site is present in human 57779, located at about amino acid residues 181 to 183 of SEQ ID NO:80.

In another embodiment, the 57779 proteins of the present invention contain at least one, and preferably two transmembrane domains. As used herein, the term “transmembrane domain” includes an amino acid sequence of about 15 amino acid residues in length which spans the plasma membrane. More preferably, a transmembrane domain includes about at least 20, 24, 25, or 30 amino acid residues and spans the plasma membrane. Transmembrane domains are rich in hydrophobic residues, and typically have an α-helical structure. In a preferred embodiment, at least 50%, 60%, 70%, 80%, 90%, 95% or more of the amino acids of a transmembrane domain are hydrophobic, e.g., leucines, isoleucines, tyrosines, or tryptophans. Transmembrane domains are described in, for example, the “7tm” entry of the PFAM on-line resource provided by Washington University at St. Louis, Mo., USA and Zagotta W. N. et al., (1996) Annual Rev. Neurosci. 19: 235-63, the contents of which are incorporated herein by reference. Two transmembrane domain are present in human 57779, located at about amino acid residues 677 to 701 and 835 to 859 of SEQ ID NO:80.

In preferred embodiments, a 57779 polypeptide or protein has at least one, and preferably two transmembrane domains, or regions which include at least 17, 20, 22, 24, or more amino acid residues and share at least 70%, 80%, 90%, 95%, 98%, 99%, or more homology with a “transmembrane domain”, e.g., a transmembrane domain of human 57779, e.g., about amino acid residues 1 to 676 of SEQ ID NO:80.

In another embodiment, a 57779 protein include at least one cytoplasmic loop, also referred to herein as a cytoplasmic domain. As used herein, a “cytoplasmic loop” includes an amino acid sequence located within a cell or within the cytoplasm of a cell. As used herein in reference to a 57779 protein, a “cytoplasmic loop” includes an amino acid sequence having a length of at least about 40-300, preferably about 80-200, more preferably about 100-150, and more preferably about 132 amino acid residues and is located within a cell or within the cytoplasm of a cell. Accordingly, the N-terminal amino acid residue of a “cytoplasmic loop” is adjacent to a C-terminal amino acid residue of a transmembrane domain in a naturally-occurring 57779 or 57779-like protein, and the C-terminal amino acid residue of a “cytoplasmic loop is adjacent to an N-terminal amino acid residue of a transmembrane domain. A cytoplasmic loop is present in human 57779, located at about amino acid residues 702-834 of SEQ ID NO:80.

In preferred embodiments, a 57779 polypeptide or protein has at least one cytoplasmic loop, or region which includes at least 40, 80, 100, 120, 130, or more amino acid residues and share at least 70%, 80%, 90%, 95%, 98%, 99%, or more homology with a “cytoplasmic loop”, e.g., a cytoplasmic loop of human 57779, e.g., about amino acid residues 702 to 834 of SEQ ID NO:80.

In another embodiment of the invention, a 57779 includes a C-terminal cytoplasmic domain. As used herein, a “C-terminal cytoplasmic domain” includes an amino acid sequence located within a cellor within the cytoplasm of a cell. As used herein with reference to a 57779 protein, a C-terminal cytoplasmic domain includes an amino acid sequence having a length of at least about 40-300, preferably about 80-200, more preferably about 100-150, more preferably about 132 amino acid residues and is located within a cell or within the cytoplasm of a cell. Accordingly, the N-terminal amino acid residue of a “C-terminal cytoplasmic domain” is adjacent to a C-terminal amino acid residue of a transmembrane domain in a naturally-occurring 57779 or 57779-like protein. For example, a C-terminal cytoplasmic domain could be produced in human 57779 by truncating the protein at about amino acid residues 834 of SEQ ID NO:80.

In preferred embodiments, a 57779 polypeptide or protein includes a C-terminal cytoplasmic domain, or region which includes at least 40, 80, 100, 120, 130, or more amino acid residues and share at least 70%, 80%, 90%, 95%, 98%, 99%, or more homology with a “cytoplasmic loop” of human 57779, e.g., about amino acid residues 702 to 834 of SEQ ID NO:80.

In yet another embodiment, the 57779 molecule can further include a signal sequence. As used herein, a “signal sequence” refers to a peptide of about 10-40 amino acid residues in length which occurs at the N-terminus of secretory and integral membrane proteins and which contains a majority of hydrophobic amino acid residues. For example, a signal sequence contains at least about 15-30 amino acid residues, preferably about 17 amino acid residues, and has at least about 40-70%, preferably about 50-65%, and more preferably about 55-60% hydrophobic amino acid residues (e.g., alanine, valine, leucine, isoleucine, phenylalanine, tyrosine, tryptophan, or proline). Such a “signal sequence”, also referred to in the art as a “signal peptide”, serves to direct a protein containing such a sequence to a lipid bilayer. For example, in one embodiment, a 57779 protein contains a signal sequence of about amino acids 8-25 of SEQ ID NO:80. The “signal sequence” can be cleaved during processing of the mature protein, resulting in a mature 57779 protein corresponding to about amino acids 26 to 873 of SEQ ID NO:80.

Accordingly in one embodiment of the invention, a 57779 molecule includes at least one, preferably two, transmembrane domains and/or at least one cytoplasmic loop, and/or at least one N-terminal extracellular domain and/or a C-terminal extracellular domain.

A 57779 family member can include at least one, two, three, four, five, or preferably six cadherin domains; at least one, two, three, four, preferably five cadherin extracellular domain signature motifs; at least one N-terminal signal sequence; at least one, preferably two transmembrane domains; and at least one intracellular domain. Furthermore, a 57779 family member can include at least one, two, three, four, five, six, seven, eight, nine, ten, or preferably eleven predicted protein kinase C phosphorylation sites; at least one, two, three, four, five, six, seven, eight, nine, ten, twelve or preferably fifteen predicted casein kinase II phosphorylation sites (PS00006); at least one, two, or preferably three predicted tyrosine kinase phosphorylation sites; at least one RGD cell attachment sequence; at least one predicted glycosaminoglycan attachment site; at least one, two, three, four, five, six, or preferably seven N-glycosylation sites, and at least one, two, three, four, five, six, or preferably seven predicted N-myristylation sites.

As the 57779 polypeptides of the invention may modulate 57779-mediated activities, they may be useful as of for developing novel diagnostic and therapeutic agents for 57779-mediated or related disorders, as described below.

As used herein, a “57779 activity”, “biological activity of 57779” or “functional activity of 57779”, refers to an activity exerted by a 57779 protein, polypeptide or nucleic acid molecule on e.g., a 57779-responsive cell or on a 57779 substrate, e.g., a protein substrate, as determined in vivo or in vitro. In one embodiment, a 57779 activity is a direct activity, such as an association with a 57779 target molecule. A “target molecule” or “binding partner” is a molecule with which a 57779 protein binds or interacts in nature. In an exemplary embodiment, 57779 is a cell surface adhesion molecules, e.g., one that engages in homotypic intermolecular interactions.

A 57779 activity can also be an indirect activity, e.g., a cellular signaling activity mediated by interaction of the 57779 protein with a 57779 receptor. Based on the above-described sequence similarities, the 57779 molecules of the present invention are predicted to have similar biological activities as protocadherin family members. For example, the 57779 proteins of the present invention can have one or more of the following activities: (1) cell-cell adhesion; (2) tissue junction formation; (3) cell motility, e.g., cell-extracellular matrix interactions; (4) integrin binding, e.g., via an RGD peptide sequence (at about residues 181 to 183 of SEQ ID NO:80); (5) calcium binding and sensing; (6) homotypic intermolecular cohesion of surface membranes; (7) signal transduction; (8) regulation of the cytoskelton, e.g., the actin cytoskelton; and (9) modulation of growth factor signaling pathways.

In addition, the 57779 molecules of the invention can be expected to function in tissues in which they are expressed. For example, human 57779 is expressed in the brain (e.g., the cortex, hypothalamus, and dorsal root ganglia), endothelial cells, ovary, and breast, as well as a variety of other tissues (see Example 11). Thus, the 57779 molecules can act as novel diagnostic targets and therapeutic agents for controlling aberrant or deficient cellular adhesion, proliferation, and/or differentiation disorders (e.g., in the brain, lung, or colon), neurological tissue and/or neurological cell disorders (e.g., brain disorders), or cardiovascular disorders (e.g., endothelial cell disorders).

Examples of “inflammatory disorders” or hematopoieitic disorders or diseases include, but are not limited to, autoimmune diseases (including, for example, diabetes mellitus, arthritis (including rheumatoid arthritis, juvenile rheumatoid arthritis, osteoarthritis, psoriatic arthritis), multiple sclerosis, encephalomyelitis, myasthenia gravis, systemic lupus erythematosis, autoimmune thyroiditis, dermatitis (including atopic dermatitis and eczematous dermatitis), psoriasis, Sjogren's Syndrome, Crohn's disease, aphthous ulcer, iritis, conjunctivitis, keratoconjunctivitis, ulcerative colitis, asthma, allergic asthma, cutaneous lupus erythematosus, scleroderma, vaginitis, proctitis, drug eruptions, leprosy reversal reactions, erythema nodosum leprosum, autoimmune uveitis, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis, Wegener's granulomatosis, chronic active hepatitis, Stevens-Johnson syndrome, idiopathic sprue, lichen planus, Graves' disease, sarcoidosis, primary biliary cirrhosis, uveitis posterior, and interstitial lung fibrosis), graft-versus-host disease, cases of transplantation, and allergy such as, atopic allergy.

Neurological disorders or disorders involving the brain include, but are not limited to, disorders involving neurons, and disorders involving glia, such as astrocytes, oligodendrocytes, ependymal cells, and microglia; cerebral edema, raised intracranial pressure and herniation, and hydrocephalus; malformations and developmental diseases, such as neural tube defects, forebrain anomalies, posterior fossa anomalies, and syringomyelia and hydromyelia; perinatal brain injury; cerebrovascular diseases, such as those related to hypoxia, ischemia, and infarction, including hypotension, hypoperfusion, and low-flow states—global cerebral ischemia and focal cerebral ischemia—infarction from obstruction of local blood supply, intracranial hemorrhage, including intracerebral (intraparenchymal) hemorrhage, subarachnoid hemorrhage and ruptured berry aneurysms, and vascular malformations, hypertensive cerebrovascular disease, including lacunar infarcts, slit hemorrhages, and hypertensive encephalopathy; infections, such as acute meningitis, including acute pyogenic (bacterial) meningitis and acute aseptic (viral) meningitis, acute focal suppurative infections, including brain abscess, subdural empyema, and extradural abscess, chronic bacterial meningoencephalitis, including tuberculosis and mycobacterioses, neurosyphilis, and neuroborreliosis (Lyme disease), viral meningoencephalitis, including arthropod-bome (Arbo) viral encephalitis, Herpes simplex virus Type 1, Herpes simplex virus Type 2, Varicalla-zoster virus (Herpes zoster), cytomegalovirus, poliomyelitis, rabies, and human immunodeficiency virus 1, including HIV-1 meningoencephalitis (subacute encephalitis), vacuolar myelopathy, AIDS-associated myopathy, peripheral neuropathy, and AIDS in children, progressive multifocal leukoencephalopathy, subacute sclerosing panencephalitis, fungal meningoencephalitis, other infectious diseases of the nervous system; transmissible spongiform encephalopathies (prion diseases); demyelinating diseases, including multiple sclerosis, multiple sclerosis variants, acute disseminated encephalomyelitis and acute necrotizing hemorrhagic encephalomyelitis, and other diseases with demyelination; degenerative diseases, such as degenerative diseases affecting the cerebral cortex, including Alzheimer disease and Pick disease, degenerative diseases of basal ganglia and brain stem, including Parkinsonism, idiopathic Parkinson disease (paralysis agitans), progressive supranuclear palsy, corticobasal degenration, multiple system atrophy, including striatonigral degenration, Shy-Drager syndrome, and olivopontocerebellar atrophy, and Huntington disease; spinocerebellar degenerations, including spinocerebellar ataxias, including Friedreich ataxia, and ataxia-telanglectasia, degenerative diseases affecting motor neurons, including amyotrophic lateral sclerosis (motor neuron disease), bulbospinal atrophy (Kennedy syndrome), and spinal muscular atrophy; inborn errors of metabolism, such as leukodystrophies, including Krabbe disease, metachromatic leukodystrophy, adrenoleukodystrophy, Pelizaeus-Merzbacher disease, and Canavan disease, mitochondrial encephalomyopathies, including Leigh disease and other mitochondrial encephalomyopathies; toxic and acquired metabolic diseases, including vitamin deficiencies such as thiamine (vitamin B1) deficiency and vitamin B12 deficiency, neurologic sequelae of metabolic disturbances, including hypoglycemia, hyperglycemia, and hepatic encephatopathy, toxic disorders, including carbon monoxide, methanol, ethanol, and radiation, including combined methotrexate and radiation-induced injury; tumors, such as gliomas, including astrocytoma, including fibrillary (diffuse) astrocytoma and glioblastoma multiforme, pilocytic astrocytoma, pleomorphic xanthoastrocytoma, and brain stem glioma, oligodendroglioma, and ependymoma and related paraventricular mass lesions, neuronal tumors, poorly differentiated neoplasms, including medulloblastoma, other parenchymal tumors, including primary brain lymphoma, germ cell tumors, and pineal parenchymal tumors, meningiomas, metastatic tumors, paraneoplastic syndromes, peripheral nerve sheath tumors, including schwannoma, neurofibroma, and malignant peripheral nerve sheath tumor (malignant schwannoma), and neurocutaneous syndromes (phakomatoses), including neurofibromotosis, including Type 1 neurofibromatosis (NF1) and TYPE 2 neurofibromatosis (NF2), tuberous sclerosis, and Von Hippel-Lindau disease.

As used herein, disorders involving the heart, or “cardiovascular disease” or a “cardiovascular disorder” includes a disease or disorder which affects the cardiovascular system, e.g., the heart, the blood vessels, and/or the blood. A cardiovascular disorder can be caused by an imbalance in arterial pressure, a malfunction of the heart, or an occlusion of a blood vessel, e.g., by a thrombus. A cardiovascular disorder includes, but is not limited to disorders such as arteriosclerosis, atherosclerosis, cardiac hypertrophy, ischemia reperfusion injury, restenosis, arterial inflammation, vascular wall remodeling, ventricular remodeling, rapid ventricular pacing, coronary microembolism, tachycardia, bradycardia, pressure overload, aortic bending, coronary artery ligation, vascular heart disease, valvular disease, including but not limited to, valvular degeneration caused by calcification, rheumatic heart disease, endocarditis, or complications of artificial valves; atrial fibrillation, long-QT syndrome, congestive heart failure, sinus node dysfunction, angina, heart failure, hypertension, atrial fibrillation, atrial flutter, pericardial disease, including but not limited to, pericardial effusion and pericarditis; cardiomyopathies, e.g., dilated cardiomyopathy or idiopathic cardiomyopathy, myocardial infarction, coronary artery disease, coronary artery spasm, ischemic disease, arrhythmia, sudden cardiac death, and cardiovascular developmental disorders (e.g., arteriovenous malformations, arteriovenous fistulae, raynaud's syndrome, neurogenic thoracic outlet syndrome, causalgia/reflex sympathetic dystrophy, hemangioma, aneurysm, cavernous angioma, aortic valve stenosis, atrial septal defects, atrioventricular canal, coarctation of the aorta, ebsteins anomaly, hypoplastic left heart syndrome, interruption of the aortic arch, mitral valve prolapse, ductus arteriosus, patent foramen ovale, partial anomalous pulmonary venous return, pulmonary atresia with ventricular septal defect, pulmonary atresia without ventricular septal defect, persistance of the fetal circulation, pulmonary valve stenosis, single ventricle, total anomalous pulmonary venous return, transposition of the great vessels, tricuspid atresia, truncus artenosus, ventricular septal defects). A cardiovasular disease or disorder also can include an endothelial cell disorder.

As used herein, an “endothelial cell disorder” includes a disorder characterized by aberrant, unregulated, or unwanted endothelial cell activity, e.g., proliferation, migration, angiogenesis, or vascularization; or aberrant expression of cell surface adhesion molecules or genes associated with angiogenesis, e.g., TIE-2, FLT and FLK. Endothelial cell disorders include tumorigenesis, tumor metastasis, psoriasis, diabetic retinopathy, endometriosis, Grave's disease, ischemic disease (e.g., atherosclerosis), and chronic inflammatory diseases (e.g., rheumatoid arthritis).

Disorders involving blood vessels include, but are not limited to, responses of vascular cell walls to injury, such as endothelial dysfunction and endothelial activation and intimal thickening; vascular diseases including, but not limited to, congenital anomalies, such as arteriovenous fistula, atherosclerosis, and hypertensive vascular disease, such as hypertension; inflammatory disease—the vasculitides, such as giant cell (temporal) arteritis, Takayasu arteritis, polyarteritis nodosa (classic), Kawasaki syndrome (mucocutaneous lymph node syndrome), microscopic polyanglitis (microscopic polyarteritis, hypersensitivity or leukocytoclastic anglitis), Wegener granulomatosis, thromboanglitis obliterans (Buerger disease), vasculitis associated with other disorders, and infectious arteritis; Raynaud disease; aneurysms and dissection, such as abdominal aortic aneurysms, syphilitic (luetic) aneurysms, and aortic dissection (dissecting hematoma); disorders of veins and lymphatics, such as varicose veins, thrombophlebitis and phlebothrombosis, obstruction of superior vena cava (superior vena cava syndrome), obstruction of inferior vena cava (inferior vena cava syndrome), and lymphangitis and lymphedema; tumors, including benign tumors and tumor-like conditions, such as hemangioma, lymphangioma, glomus tumor (glomangioma), vascular ectasias, and bacillary angiomatosis, and intermediate-grade (borderline low-grade malignant) tumors, such as Kaposi sarcoma and hemangloendothelioma, and malignant tumors, such as angiosarcoma and hemangiopericytoma; and pathology of therapeutic interventions in vascular disease, such as balloon angioplasty and related techniques and vascular replacement, such as coronary artery bypass graft surgery.

Examples of cellular proliferative and/or differentiative disorders include cancer, e.g., carcinoma, sarcoma, metastatic disorders or hematopoietic neoplastic disorders, e.g., leukemias. A metastatic tumor can arise from a multitude of primary tumor types, including but not limited to those of prostate, colon, lung, breast and liver origin.

As used herein, the terms “cancer”, “hyperproliferative” and “neoplastic” refer to cells having the capacity for autonomous growth. Examples of such cells include cells having an abnormal state or condition characterized by rapidly proliferating cell growth. Hyperproliferative and neoplastic disease states may be categorized as pathologic, i.e., characterizing or constituting a disease state, or may be categorized as non-pathologic, i.e., a deviation from normal but not associated with a disease state. The term is meant to include all types of cancerous growths or oncogenic processes, metastatic tissues or malignantly transformed cells, tissues, or organs, irrespective of histopathologic type or stage of invasiveness. “Pathologic hyperproliferative” cells occur in disease states characterized by malignant tumor growth. Examples of non-pathologic hyperproliferative cells include proliferation of cells associated with wound repair.

The terms “cancer” or “neoplasms” include malignancies of the various organ systems, such as affecting lung, breast, thyroid, lymphoid, gastrointestinal, and genito-urinary tract, as well as adenocarcinomas which include malignancies such as most colon cancers, renal-cell carcinoma, prostate cancer and/or testicular tumors, non-small cell carcinoma of the lung, cancer of the small intestine and cancer of the esophagus.

The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. Exemplary carcinomas include those forming from tissue of the cervix, lung, prostate, breast, head and neck, colon and ovary. The term also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures.

The term “sarcoma” is art recognized and refers to malignant tumors of mesenchymal derivation.

Examples of cellular proliferative and/or differentiative disorders of the colon include, but are not limited to, non-neoplastic polyps, adenomas, familial syndromes, colorectal carcinogenesis, colorectal carcinoma, and carcinoid tumors.

Examples of cellular proliferative and/or differentiative disorders of the liver include, but are not limited to, nodular hyperplasias, adenomas, and malignant tumors, including primary carcinoma of the liver and metastatic tumors.

Examples of cellular proliferative and/or differentiative disorders of the breast include, but are not limited to, proliferative breast disease including, e.g., epithelial hyperplasia, sclerosing adenosis, and small duct papillomas; tumors, e.g., stromal tumors such as fibroadenoma, phyllodes tumor, and sarcomas, and epithelial tumors such as large duct papilloma; carcinoma of the breast including in situ (noninvasive) carcinoma that includes ductal carcinoma in situ (including Paget's disease) and lobular carcinoma in situ, and invasive (infiltrating) carcinoma including, but not limited to, invasive ductal carcinoma, invasive lobular carcinoma, medullary carcinoma, colloid (mucinous) carcinoma, tubular carcinoma, and invasive papillary carcinoma, and miscellaneous malignant neoplasms. Disorders in the male breast include, but are not limited to, gynecomastia and carcinoma.

Examples of cellular proliferative and/or differentiative disorders of the lung include, but are not limited to, bronchogenic carcinoma, including paraneoplastic syndromes, bronchioloalveolar carcinoma, neuroendocrine tumors, such as bronchial carcinoid, miscellaneous tumors, and metastatic tumors; pathologies of the pleura, including inflammatory pleural effusions, noninflammatory pleural effusions, pneumothorax, and pleural tumors, including solitary fibrous tumors (pleural fibroma) and malignant mesothelioma.

Additional examples of proliferative disorders include hematopoietic neoplastic disorders. As used herein, the term “hematopoietic neoplastic disorders” includes diseases involving hyperplastic/neoplastic cells of hematopoietic origin. A hematopoietic neoplastic disorder can arise from myeloid, lymphoid or erythroid lineages, or precursor cells thereof. Preferably, the diseases arise from poorly differentiated acute leukemias, e.g., erythroblastic leukemia and acute megakaryoblastic leukemia. Additional exemplary myeloid disorders include, but are not limited to, acute promyeloid leukemia (APML), acute myelogenous leukemia (AML) and chronic myelogenous leukemia (CML) (reviewed in Vaickus, L. (1991) Crit Rev. in Oncol./Hemotol. 11:267-97); lymphoid malignancies include, but are not limited to acute lymphoblastic leukemia (ALL) which includes B-lineage ALL and T-lineage ALL, chronic lymphocytic leukemia (CLL), prolymphocytic leukemia (PLL), hairy cell leukemia (HLL) and Waldenstrom's macroglobulinemia (WM). Additional forms of malignant lymphomas include, but are not limited to non-Hodgkin lymphoma and variants thereof, peripheral T cell lymphomas, adult T cell leukemia/lymphoma (ATL), cutaneous T-cell lymphoma (CTCL), large granular lymphocytic leukemia (LGF), Hodgkin's disease and Reed-Sternberg disease.

Disorders involving the small intestine include the malabsorption syndromes such as, celiac sprue, tropical sprue (postinfectious sprue), whipple disease, disaccharidase (lactase) deficiency, abetalipoproteinemia, and tumors of the small intestine including adenomas and adenocarcinoma.

Disorders involving the pancreas include those of the exocrine pancreas such as congenital anomalies, including but not limited to, ectopic pancreas; pancreatitis, including but not limited to, acute pancreatitis; cysts, including but not limited to, pseudocysts; tumors, including but not limited to, cystic tumors and carcinoma of the pancreas; and disorders of the endocrine pancreas such as, diabetes mellitus; islet cell tumors, including but not limited to, insulinomas, gastrinomas, and other rare islet cell tumors.

Disorders involving red blood cells include, but are not limited to, anemias, such as hemolytic anemias, including hereditary spherocytosis, hemolytic disease due to erythrocyte enzyme defects: glucose-6-phosphate dehydrogenase deficiency, sickle cell disease, thalassemia syndromes, paroxysmal nocturnal hemoglobinuria, immunohemolytic anemia, and hemolytic anemia resulting from trauma to red cells; and anemias of diminished erythropoiesis, including megaloblastic anemias, such as anemias of vitamin B12 deficiency: pernicious anemia, and anemia of folate deficiency, iron deficiency anemia, anemia of chronic disease, aplastic anemia, pure red cell aplasia, and other forms of marrow failure.

Disorders related to reduced platelet number, thrombocytopenia, include idiopathic thrombocytopenic purpura, including acute idiopathic thrombocytopenic purpura, drug-induced thrombocytopenia, HIV-associated thrombocytopenia, and thrombotic microangiopathies: thrombotic thrombocytopenic purpura and hemolytic-uremic syndrome.

Examples of viral diseases include, but are not limited to, Hepatitis B, Hepatitis C and Herpes Simplex Virus (HSV). Modulators of 33489 activity, in particular, could be used to control viral diseases. The modulators can be used in the treatment and/or diagnosis of viral infected tissue or virus-associated tissue fibrosis, especially liver and liver fibrosis. Also, 33489 modulators can be used in the treatment and/or diagnosis of virus-associated carcinoma, especially hepatocellular cancer.

The 23566, 33489, and 57779 proteins, fragments thereof, and derivatives and other variants of the sequences in SEQ ID NO:74, SEQ ID NO:77, or SEQ ID NO:80 thereof are collectively referred to as “polypeptides or proteins of the invention” or “23566”, “33489”, and “57779” polypeptides or proteins. Nucleic acid molecules encoding such polypeptides or proteins are collectively referred to as “nucleic acids of the invention” or “23566”, “33489”, and “57779” nucleic acids. 23566, 33489, and 57779 molecules refer to 23566, 33489, and 57779 nucleic acids, polypeptides, and antibodies.

As used herein, the term “nucleic acid molecule” includes DNA molecules (e.g., a cDNA or genomic DNA), RNA molecules (e.g., an mRNA) and analogs of the DNA or RNA. A DNA or RNA analog can be synthesized from nucleotide analogs. The nucleic acid molecule can be single-stranded or double-stranded, but preferably is double-stranded DNA.

The term “isolated nucleic acid molecule” or “purified nucleic acid molecule” includes nucleic acid molecules that are separated from other nucleic acid molecules present in the natural source of the nucleic acid. For example, with regards to genomic DNA, the term “isolated” includes nucleic acid molecules which are separated from the chromosome with which the genomic DNA is naturally associated. Preferably, an “isolated” nucleic acid is free of sequences which naturally flank the nucleic acid (i.e., sequences located at the 5′ and/or 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb or 0.1 kb of 5′ and/or 3′ nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material, or culture medium when produced by recombinant techniques, or substantially free of chemical precursors or other chemicals when chemically synthesized.

As used herein, the term “hybridizes under low stringency, medium stringency, high stringency, or very high stringency conditions” describes conditions for hybridization and washing. Guidance for performing hybridization reactions can be found in Current Protocols in Molecular Biology, John Wiley & Sons, N.Y. (1989), 6.3.1-6.3.6, which is incorporated by reference. Aqueous and nonaqueous methods are described in that reference and either can be used. Specific hybridization conditions referred to herein are as follows: 1) low stringency hybridization conditions in 6× sodium chloride/sodium citrate (SSC) at about 45° C., followed by two washes in 0.2× SSC, 0.1% SDS at least at 50° C. (the temperature of the washes can be increased to 55° C. for low stringency conditions); 2) medium stringency hybridization conditions in 6× SSC at about 45° C., followed by one or more washes in 0.2× SSC, 0.1% SDS at 60° C.; 3) high stringency hybridization conditions in 6× SSC at about 45° C., followed by one or more washes in 0.2× SSC, 0.1% SDS at 65° C.; and preferably 4) very high stringency hybridization conditions are 0.5M sodium phosphate, 7% SDS at 65° C., followed by one or more washes at 0.2× SSC, 1% SDS at 65° C. Very high stringency conditions (4) are the preferred conditions and the ones that should be used unless otherwise specified.

Preferably, an isolated nucleic acid molecule of the invention that hybridizes under a stringency condition described herein to the sequence of SEQ ID NO:73, SEQ ID NO:75, or SEQ ID NO:76, SEQ ID NO:78, SEQ ID NO:79, or SEQ ID NO:81, corresponds to a naturally-occurring nucleic acid molecule.

As used herein, a “naturally-occurring” nucleic acid molecule refers to an RNA or DNA molecule having a nucleotide sequence that occurs in nature. For example a naturally occurring nucleic acid molecule can encode a natural protein.

As used herein, the terms “gene” and “recombinant gene” refer to nucleic acid molecules which include at least an open reading frame encoding a 23566, 33489, or 57779 protein. The gene can optionally further include non-coding sequences, e.g., regulatory sequences and introns. Preferably, a gene encodes a mammalian 23566, 33489, or 57779 protein or derivative thereof.

An “isolated” or “purified” polypeptide or protein is substantially free of cellular material or other contaminating proteins from the cell or tissue source from which the protein is derived, or substantially free from chemical precursors or other chemicals when chemically synthesized. “Substantially free” means that a preparation of 23566, 33489, or 57779 protein is at least 10% pure. In a preferred embodiment, the preparation of 23566, 33489, or 57779 protein has less than about 30%, 20%, 10% and more preferably 5% (by dry weight), of non-23566, 33489, or 57779 protein (also referred to herein as a “contaminating protein”), or of chemical precursors or non-23566, 33489, or 57779 chemicals. When the 23566, 33489, or 57779 protein or biologically active portion thereof is recombinantly produced, it is also preferably substantially free of culture medium, i.e., culture medium represents less than about 20%, more preferably less than about 10%, and most preferably less than about 5% of the volume of the protein preparation. The invention includes isolated or purified preparations of at least 0.01, 0.1, 1.0, and 10 milligrams in dry weight.

A “non-essential” amino acid residue is a residue that can be altered from the wild-type sequence of 23566, 33489, or 57779 without abolishing or substantially altering a 23566, 33489, or 57779 activity. Preferably the alteration does not substantially alter the 23566, 33489, or 57779 activity, e.g., the activity is at least 20%, 40%, 60%, 70% or 80% of wild-type. An “essential” amino acid residue is a residue that, when altered from the wild-type sequence of 23566, 33489, or 57779, results in abolishing a 23566, 33489, or 57779 activity such that less than 20% of the wild-type activity is present. For example, conserved amino acid residues in 23566, 33489, or 57779 are predicted to be particularly unamenable to alteration.

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in a 23566, 33489, or 57779 protein is preferably replaced with another amino acid residue from the same side chain family.

Alternatively, in another embodiment, mutations can be introduced randomly along all or part of a 23566, 33489, or 57779 coding sequence, such as by saturation mutagenesis, and the resultant mutants can be screened for 23566, 33489, or 57779 biological activity to identify mutants that retain activity. Following mutagenesis of SEQ ID NO:73, SEQ ID NO:75, SEQ ID NO:76, SEQ ID NO:78, SEQ ID NO:79, or SEQ ID NO:81, the encoded protein can be expressed recombinantly and the activity of the protein can be determined.

As used herein, a “biologically active portion” of a 23566, 33489, or 57779 protein includes a fragment of a 23566, 33489, or 57779 protein which participates in an interaction, e.g., an intramolecular or an inter-molecular interaction. An inter-molecular interaction can be a specific binding interaction or an enzymatic interaction (e.g., the interaction can be transient and a covalent bond is formed or broken). An inter-molecular interaction can be between a 23566, 33489, or 57779 molecule and a non-23566, 33489, or 57779 molecule or between a first 23566, 33489, or 57779 molecule and a second 23566, 33489, or 57779 molecule (e.g., a dimerization interaction). Biologically active portions of a 23566, 33489, or 57779 protein include peptides comprising amino acid sequences sufficiently homologous to or derived from the amino acid sequence of the 23566, 33489, or 57779 protein, e.g., the amino acid sequence shown in SEQ ID NO:74, SEQ ID NO:77, OR SEQ ID NO:80, which include less amino acids than the full length 23566, 33489, or 57779 proteins, and exhibit at least one activity of a 23566, 33489, or 57779 protein. Typically, biologically active portions comprise a domain or motif with at least one activity of the 23566, 33489, or 57779 protein, e.g., peptide hydrolysis activity (e.g., carboxy terminal peptide hydrolysis activity), scramblase activity, or cellular adhesive activity (e.g., homotypic cellular adhesion). A biologically active portion of a 23566, 33489, or 57779 protein can be a polypeptide which is, for example, 10, 25, 50, 100, 200 or more amino acids in length. Biologically active portions of a 23566, 33489, or 57779 protein can be used as targets for developing agents which modulate a 23566, 33489, or 57779 mediated activity, e.g., peptide hydrolysis activity (e.g., carboxy terminal peptide hydrolysis activity), scramblase activity, or cellular adhesive activity (e.g., homotypic cellular adhesion).

Calculations of homology or sequence identity between sequences (the terms are used interchangeably herein) are performed as follows.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, 60%, and even more preferably at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”).

The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch ((1970) J. Mol. Biol. 48:444-453) algorithm which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. A particularly preferred set of parameters (and the one that should be used unless otherwise specified) are a Blossum 62 scoring matrix with a gap penalty of 12, a gap extend penalty of 4, and a frameshift gap penalty of 5.

The percent identity between two amino acid or nucleotide sequences can be determined using the algorithm of E. Meyers and W. Miller ((1989) CABIOS, 4:11-17) which has been incorporated into the ALIGN program (version 2.0), using a PAM120 weight residue table, a gap length penalty of 12 and a gap penalty of 4.

The nucleic acid and protein sequences described herein can be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to 23566, 33489, or 57779 nucleic acid molecules of the invention. BLAST protein searches can be performed with the XBLAST program, score=50, wordlength=3 to obtain amino acid sequences homologous to 23566, 33489, or 57779 protein molecules of the invention. To obtain gapped alignments for comparison purposes, Gapped BLAST can be utilized as described in Altschul et al., (1997) Nucleic Acids Res. 25:3389-3402. When utilizing BLAST and Gapped BLAST programs, the default parameters of the respective programs (e.g., XBLAST and NBLAST) can be used. See http://www.ncbi.nlm.nih.gov.

Particularly preferred 23566, 33489, or 57779 polypeptides of the present invention have an amino acid sequence substantially identical to the amino acid sequence of SEQ ID NO:74, SEQ ID NO:77, or SEQ ID NO:80. In the context of an amino acid sequence, the term “substantially identical” is used herein to refer to a first amino acid that contains a sufficient or minimum number of amino acid residues that are i) identical to, or ii) conservative substitutions of aligned amino acid residues in a second amino acid sequence such that the first and second amino acid sequences can have a common structural domain and/or common functional activity. For example, amino acid sequences that contain a common structural domain having at least about 60%, or 65% identity, likely 75% identity, more likely 85%, 90%. 91%, 92%, 93%, 94%, 95%, 96%, 97%, 98% or 99% identity to SEQ ID NO:74, SEQ ID NO:77, or SEQ ID NO:80 are termed substantially identical.

In the context of nucleotide sequence, the term “substantially identical” is used herein to refer to a first nucleic acid sequence that contains a sufficient or minimum number of nucleotides that are identical to aligned nucleotides in a second nucleic acid sequence such that the first and second nucleotide sequences encode a polypeptide having common functional activity, or encode a common structural polypeptide domain or a common functional polypeptide activity. For example, nucleotide sequences having at least about 60%, or 65% identity, likely 75% identity, more likely 85%, 90%. 91%, 92%, 93%, 94%, 95%, 96%, 97%, 98% or 99% identity to SEQ ID NO:73, SEQ ID NO:75, SEQ ID NO:76, SEQ ID NO:78, SEQ ID NO:79, or SEQ ID NO:81 are termed substantially identical.

“Misexpression or aberrant expression”, as used herein, refers to a non-wildtype pattern of gene expression at the RNA or protein level. It includes: expression at non-wild type levels, i.e., over- or under-expression; a pattern of expression that differs from wild type in terms of the time or stage at which the gene is expressed, e.g., increased or decreased expression (as compared with wild type) at a predetermined developmental period or stage; a pattern of expression that differs from wild type in terms of altered, e.g., increased or decreased, expression (as compared with wild type) in a predetermined cell type or tissue type; a pattern of expression that differs from wild type in terms of the splicing size, translated amino acid sequence, post-transitional modification, or biological activity of the expressed polypeptide; a pattern of expression that differs from wild type in terms of the effect of an environmental stimulus or extracellular stimulus on expression of the gene, e.g., a pattern of increased or decreased expression (as compared with wild type) in the presence of an increase or decrease in the strength of the stimulus.

“Subject,” as used herein, refers to human and non-human animals. The term “non-human animals” of the invention includes all vertebrates, e.g., mammals, such as non-human primates (particularly higher primates), sheep, dog, rodent (e.g., mouse or rat), guinea pig, goat, pig, cat, rabbits, cow, and non-mammals, such as chickens, amphibians, reptiles, etc. In a preferred embodiment, the subject is a human. In another embodiment, the subject is an experimental animal or animal suitable as a disease model.

A “purified preparation of cells”, as used herein, refers to an in vitro preparation of cells. In the case cells from multicellular organisms (e.g., plants and animals), a purified preparation of cells is a subset of cells obtained from the organism, not the entire intact organism. In the case of unicellular microorganisms (e.g., cultured cells and microbial cells), it consists of a preparation of at least 10% and more preferably 50% of the subject cells.

Various aspects of the invention are described in further detail below.

Isolated Nucleic Acid Molecules of 23566, 33489, and 57779

In one aspect, the invention provides, an isolated or purified, nucleic acid molecule that encodes a 23566, 33489, or 57779 polypeptide described herein, e.g., a full-length 23566, 33489, or 57779 protein or a fragment thereof, e.g., a biologically active portion of 23566, 33489, or 57779 protein. Also included is a nucleic acid fragment suitable for use as a hybridization probe, which can be used, e.g., to identify a nucleic acid molecule encoding a polypeptide of the invention, 23566, 33489, or 57779 mRNA, and fragments suitable for use as primers, e.g., PCR primers for the amplification or mutation of nucleic acid molecules.

In one embodiment, an isolated nucleic acid molecule of the invention includes the nucleotide sequence shown in SEQ ID NO:73, SEQ ID NO:76, or SEQ ID NO:79, or a portion of any of these nucleotide sequences. In one embodiment, the nucleic acid molecule includes sequences encoding the human 23566, 33489, or 57779 protein (i.e., “the coding region” of SEQ ID NO:73, SEQ ID NO:76, or SEQ ID NO:79, as shown in SEQ ID NO:75, SEQ ID NO:78, and SEQ ID NO:81, respectively), as well as 5′ untranslated sequences. Alternatively, the nucleic acid molecule can include only the coding region of SEQ ID NO:73, SEQ ID NO:76, or SEQ ID NO:79 (e.g., SEQ ID NO:75, SEQ ID NO:78, or SEQ ID NO:81) and, e.g., no flanking sequences which normally accompany the subject sequence. In another embodiment, the nucleic acid molecule encodes a sequence corresponding to a fragment of the protein from about amino acid residues 67 to 444 of SEQ ID NO:74, or about amino acid residues 25 to 873 of SEQ ID NO:80.

In another embodiment, an isolated nucleic acid molecule of the invention includes a nucleic acid molecule which is a complement, e.g., a full complement, of the nucleotide sequence shown in SEQ ID NO:73, SEQ ID NO:75, SEQ ID NO:76, SEQ ID NO:78, SEQ ID NO:79, or SEQ ID NO:81, or a portion of any of these nucleotide sequences. In other embodiments, the nucleic acid molecule of the invention is sufficiently complementary to the nucleotide sequence shown in SEQ ID NO:73, SEQ ID NO:75, SEQ ID NO:76, SEQ ID NO:78, SEQ ID NO:79, or SEQ ID NO:81, such that it can hybridize (e.g., under a stringency condition described herein) to the nucleotide sequence shown in SEQ ID NO:73, SEQ ID NO:75, SEQ ID NO:76, SEQ ID NO:78, SEQ ID NO:79 or SEQ ID NO:81, thereby forming a stable duplex.

In one embodiment, an isolated nucleic acid molecule of the present invention includes a nucleotide sequence which is at least about: 60%, 65%, 70%, 75%, 80%, 85%, 90%, 91%, 92%, 93%, 94%, 95%, 96%, 97%, 98%, 99%, or more homologous to the entire length of the nucleotide sequence shown in SEQ ID NO:73, SEQ ID NO:76, OR SEQ ID NO:79 or SEQ ID NO:75, SEQ ID NO:78, or SEQ ID NO:81, or a portion, preferably of the same length, of any of these nucleotide sequences.

23566 Nucleic Acid Fragments

A nucleic acid molecule of the invention can include only a portion of the nucleic acid sequence of SEQ ID NO:73 or SEQ ID NO:75. For example, such a nucleic acid molecule can include a fragment which can be used as a probe or primer or a fragment encoding a portion of a 23566 protein, e.g., an immunogenic or biologically active portion of a 23566 protein. A fragment can comprise those nucleotides of SEQ ID NO:73, which encode a zinc carboxypeptidase domain of human 23566. The nucleotide sequence determined from the cloning of the 23566 gene allows for the generation of probes and primers designed for use in identifying and/or cloning other 23566 family members, or fragments thereof, as well as 23566 homologues, or fragments thereof, from other species.

In another embodiment, a nucleic acid includes a nucleotide sequence that includes part, or all, of the coding region and extends into either (or both) the 5′ or 3′ noncoding region. Other embodiments include a fragment which includes a nucleotide sequence encoding an amino acid fragment described herein. Nucleic acid fragments can encode a specific domain or site described herein or fragments thereof, particularly fragments thereof which are at least 50 amino acids in length e.g. at least 75, 100, 150, 200, 250, 300, 350, 400, or 440 amino acids in length. Preferably, the nucleic acid fragments encode a specific domain or fragment thereof, wherein the domain or fragment is at least 311, 320, 340, 360, 380, 400, 420, or 440 amino acids in length. Fragments also include nucleic acid sequences corresponding to specific amino acid sequences described above or fragments thereof. Nucleic acid fragments should not to be construed as encompassing those fragments that may have been disclosed prior to the invention.

A nucleic acid fragment can include a sequence corresponding to a domain, region, or functional site described herein. A nucleic acid fragment can also include one or more domain, region, or functional site described herein. Thus, for example, a 23566 nucleic acid fragment can include a sequence corresponding to a zinc carboxypeptidase domain, e.g., about amino acid residues 180 to 443 of SEQ ID NO:74, or a zinc carboxylpeptidase zinc-binding region 1 signature motif, e.g., about amino acid residues 229 to 251 of SEQ ID NO:74.

23566 probes and primers are provided. Typically a probe/primer is an isolated or purified oligonucleotide. The oligonucleotide typically includes a region of nucleotide sequence that hybridizes under a stringency condition described herein to at least about 7, 12 or 15, preferably about 20 or 25, more preferably about 30, 35, 40, 45, 50, 55, 60, 65, or 75 consecutive nucleotides of a sense or antisense sequence of SEQ ID NO:73 or SEQ ID NO:75, or of a naturally occurring allelic variant or mutant of SEQ ID NO:73 or SEQ ID NO:75. Preferably, an oligonucleotide is less than about 200, 150, 120, or 100 nucleotides in length.

In one embodiment, the probe or primer is attached to a solid support, e.g., a solid support described herein.

One exemplary kit of primers includes a forward primer that anneals to the coding strand and a reverse primer that anneals to the non-coding strand. The forward primer can anneal to the start codon, e.g., the nucleic acid sequence encoding amino acid residue 1 of SEQ ID NO:74. The reverse primer can anneal to the ultimate codon, e.g., the codon immediately before the stop codon, e.g., the codon encoding amino acid residue 444 of SEQ ID NO:74. In a preferred embodiment, the annealing temperatures of the forward and reverse primers differ by no more than 5, 4, 3, or 2° C.

In a preferred embodiment the nucleic acid is a probe which is at least 10, 12, 15, 18, 20 and less than 200, more preferably less than 100, or less than 50, nucleotides in length. It should be identical, or differ by 1, or 2, or less than 5 or 10 nucleotides, from a sequence disclosed herein. If alignment is needed for this comparison the sequences should be aligned for maximum homology. “Looped” out sequences from deletions or insertions, or mismatches, are considered differences.

A probe or primer can be derived from the sense or anti-sense strand of a nucleic acid which encodes: a zinc carboxypeptidase domain of 23566, e.g., located at about amino acid residues 180 to 443 of SEQ ID NO:74; a zinc carboxypeptidase zinc-binding region 1 signature motif of 23566, e.g., located at about amino acid residues 229 to 251 of SEQ ID NO:74; predicted N-glycosylation sites of 23566, e.g., located at about amino acids 304 to 307, 317 to 320 or 381 to 384 of SEQ ID NO:74; predicted protein kinase C phosphorylation sites of 23566, e.g., located at about amino acids 46 to 48, 194 to 196, 291 to 293, 354 to 356 or 423 to 425 of SEQ ID NO:74; predicted casein kinase II phosphorylation sites of 23566, e.g., located at about amino 341 to 344, 350 to 353, 354 to 357, 412 to 415 or 423 to 426 of SEQ ID NO:74; predicted tyrosine kinase phosphorylation sites of 23566, e.g., located at about amino acid 132 to 139 of SEQ ID NO:74; predicted N-myristylation sites of 23566, e.g., located at about amino acid 14 to 19, 305 to 310, 324 to 329, 393 to 398 or 432 to 437 of SEQ ID NO:74; and predicted amidation sites of 23566, e.g., located at about amino acid 37 to 40 of SEQ ID NO:74.

In another embodiment a set of primers is provided, e.g., primers suitable for use in a PCR, which can be used to amplify a selected region of a 23566 sequence, e.g., a domain, region, site or other sequence described herein. The primers should be at least 5, 10, or 50 base pairs in length and less than 100, or less than 200, base pairs in length. The primers should be identical, or differs by one base from a sequence disclosed herein or from a naturally occurring variant. For example, primers suitable for amplifying all or a portion of any of the following regions are provided: a zinc carboxypeptidase domain from about amino acid 180 to 443 of SEQ ID NO:74; and a zinc carboxypeptidase zinc-binding region 1 signature domain from about amino acid 229 to 251 of SEQ ID NO:74.

A nucleic acid fragment can encode an epitope bearing region of a polypeptide described herein.

A nucleic acid fragment encoding a “biologically active portion of a 23566 polypeptide” can be prepared by isolating a portion of the nucleotide sequence of SEQ ID NO:73 or SEQ ID NO:75, which encodes a polypeptide having a 23566 biological activity (e.g., the biological activities of the 23566 proteins are described herein), expressing the encoded portion of the 23566 protein (e.g., by recombinant expression in vitro) and assessing the activity of the encoded portion of the 23566 protein. For example, a nucleic acid fragment encoding a biologically active portion of 23566 includes a zinc carboxypeptidase domain, e.g., amino acid residues about 180 to 443 of SEQ ID NO:74. A nucleic acid fragment encoding a biologically active portion of a 23566 polypeptide may comprise a nucleotide sequence that is greater than 300 or more nucleotides in length.

In preferred embodiments, a nucleic acid includes a nucleotide sequence which is about 300, 400, 500, 600, 700, 800, 900, 1000, 1100, 1200, 1300, 1400, 1500, 1600, or more nucleotides in length and hybridizes under a stringency condition described herein to a nucleic acid molecule of SEQ ID NO:73, or SEQ ID NO:75.

In a preferred embodiment, a nucleic acid fragment differs by at least 1, 2, 3, 10, 20, or more nucleotides from the sequence of Genbank accession number AX083139, F81728, X51497 or C00454, or SEQ ID NOs:1, 2, 3, 5, 23, or 26 of WO2001157265. Differences can include differing in length or sequence identity. For example, a nucleic acid fragment can: include one or more nucleotides from SEQ ID NO:73 or SEQ ID NO:75 located outside the region of nucleotides 1 to 322, 597 to 1602, 133 to 143, or 589 to 913; not include all of the nucleotides of AX083139, F81728, X51497 or C00454, or SEQ ID NOs:1, 2, 3, 5, 23, or 26 of WO2001157265, e.g., can be one or more nucleotides shorter (at one or both ends) than the sequence of AX083139, F81728, X51497 or C00454, or SEQ ID NOs:1, 2, 3, 5, 23, or 26 of WO2001157265; or can differ by one or more nucleotides in the region of overlap.

33489 Nucleic Acid Fragments

A nucleic acid molecule of the invention can include only a portion of the nucleic acid sequence of SEQ ID NO:76 or SEQ ID NO:78. For example, such a nucleic acid molecule can include a fragment which can be used as a probe or primer or a fragment encoding a portion of a 33489 protein, e.g., an immunogenic or biologically active portion of a 33489 protein. A fragment can comprise those nucleotides of SEQ ID NO:76, which encode a scramblase domain of human 33489. The nucleotide sequence determined from the cloning of the 33489 gene allows for the generation of probes and primers designed for use in identifying and/or cloning other 33489 family members, or fragments thereof, as well as 33489 homologues, or fragments thereof, from other species.

In another embodiment, a nucleic acid includes a nucleotide sequence that includes part, or all, of the coding region and extends into either (or both) the 5′ or 3′ noncoding region. Other embodiments include a fragment which includes a nucleotide sequence encoding an amino acid fragment described herein. Nucleic acid fragments can encode a specific domain or site described herein or fragments thereof, particularly fragments thereof which are at least 50, 75, 100, 125, 150, 175, 200, 225, 250, or 275 amino acids in length. Preferably, the nucleic acid fragments encode a specific domain or fragment thereof, wherein the domain or fragment is at least 100 amino acids in length. Fragments also include nucleic acid sequences corresponding to specific amino acid sequences described above or fragments thereof. Nucleic acid fragments should not to be construed as encompassing those fragments that may have been disclosed prior to the invention.

A nucleic acid fragment can include a sequence corresponding to a domain, region, or functional site described herein. A nucleic acid fragment can also include one or more domain, region, or functional site described herein. Thus, for example, a 33489 nucleic acid fragment can include a sequence corresponding to a scramblase domain of 33489, a transmembrane domain of 33489, an N-terminal cytoplasmic domain of 33489, or a C-terminal extracellular domain of 33489.

33489 probes and primers are provided. Typically a probe/primer is an isolated or purified oligonucleotide. The oligonucleotide typically includes a region of nucleotide sequence that hybridizes under a stringency condition described herein to at least about 7, 12 or 15, preferably about 20 or 25, more preferably about 30, 35, 40, 45, 50, 55, 60, 65, or 75 consecutive nucleotides of a sense or antisense sequence of SEQ ID NO:76 or SEQ ID NO:78, or of a naturally occurring allelic variant or mutant of SEQ ID NO:76 or SEQ ID NO:78. Preferably, an oligonucleotide is less than about 200, 150, 120, or 100 nucleotides in length.

In one embodiment, the probe or primer is attached to a solid support, e.g., a solid support described herein.

One exemplary kit of primers includes a forward primer that anneals to the coding strand and a reverse primer that anneals to the non-coding strand. The forward primer can anneal to the start codon, e.g., the nucleic acid sequence encoding amino acid residue 1 of SEQ ID NO:77. The reverse primer can anneal to the ultimate codon, e.g., the codon immediately before the stop codon, e.g., the codon encoding amino acid residue 295 of SEQ ID NO:74. In a preferred embodiment, the annealing temperatures of the forward and reverse primers differ by no more than 5, 4, 3, or 2° C.

In a preferred embodiment the nucleic acid is a probe which is at least 10, 12, 15, 18, 20 and less than 200, more preferably less than 100, or less than 50, nucleotides in length. It should be identical, or differ by 1, or 2, or less than 5 or 10 nucleotides, from a sequence disclosed herein. If alignment is needed for this comparison the sequences should be aligned for maximum homology. “Looped” out sequences from deletions or insertions, or mismatches, are considered differences.

A probe or primer can be derived from the sense or anti-sense strand of a nucleic acid which encodes, e.g., a scramblase domain of 33489 (e.g., about amino acid residues 103 to 285 of SEQ ID NO:77), an N-terminal cytoplasmic domain of 33489 (e.g., about amino acid residues 1 to 265 of SEQ ID NO:77), a transmembrane domain of 33489 (e.g., about amino acid residues 266 to 282 of SEQ ID NO:77), or a C-terminal extracellular domain of 33489 (e.g., about amino acid residues 283 to 295 of SEQ ID NO:77).

In another embodiment a set of primers is provided, e.g., primers suitable for use in a PCR, which can be used to amplify a selected region of a 33489 sequence, e.g., a domain, region, site or other sequence described herein. The primers should be at least 5, 10, or 50 base pairs in length and less than 100, or less than 200, base pairs in length. The primers should be identical, or differs by one base from a sequence disclosed herein or from a naturally occurring variant. For example, primers suitable for amplifying all or a portion of any of the following regions are provided: a scramblase domain of 33489 (e.g., about amino acid residues 103 to 285 of SEQ ID NO:77), an N-terminal cytoplasmic domain of 33489 (e.g., about amino acid residues 1 to 265 of SEQ ID NO:77), a transmembrane domain of 33489 (e.g., about amino acid residues 266 to 282 of SEQ ID NO:77), or a C-terminal extracellular domain of 33489 (e.g., about amino acid residues 283 to 295 of SEQ ID NO:77).

A nucleic acid fragment can encode an epitope bearing region of a polypeptide described herein.

A nucleic acid fragment encoding a “biologically active portion of a 33489 polypeptide” can be prepared by isolating a portion of the nucleotide sequence of SEQ ID NO:76 or SEQ ID NO:78, which encodes a polypeptide having a 33489 biological activity (e.g., the biological activities of the 33489 proteins are described herein), expressing the encoded portion of the 33489 protein (e.g., by recombinant expression in vitro) and assessing the activity of the encoded portion of the 33489 protein. For example, a nucleic acid fragment encoding a biologically active portion of 33489 includes a scramblase domain (e.g., amino acid residues about 103 to 285 of SEQ ID NO:77), an N-terminal cytoplasmic domain of 33489 (e.g., about amino acid residues 1 to 265 of SEQ ID NO:77), a transmembrane domain of 33489 (e.g., about amino acid residues 266 to 282 of SEQ ID NO:77), or a C-terminal extracellular domain of 33489 (e.g., about amino acid residues 283 to 295 of SEQ ID NO:77). A nucleic acid fragment encoding a biologically active portion of a 33489 polypeptide, may comprise a nucleotide sequence which is greater than 300 or more nucleotides in length.

In preferred embodiments, a nucleic acid includes a nucleotide sequence which is about 300, 400, 500, 600, 700, 800, 900, 1000, 1100, 1200, 1300, 1400, 1500, 1560, 1600, 1700 or more nucleotides in length and hybridizes under a stringency condition described herein to a nucleic acid molecule of SEQ ID NO:76 or SEQ ID NO:78.

In a preferred embodiment, a nucleic acid fragment differs by at least 1, 2, 3, 10, 20, or more nucleotides from the sequence of GenBank™ accession number BE736451, BE792564, AW239215, AF159442, or AF097738. Differences can include differing in length or sequence identity. For example, a nucleic acid fragment can: include one or more nucleotides from SEQ ID NO:76 or SEQ ID NO:78 located outside the region of nucleotides 433-892, 436-1030, 41-658, 143-1733, 1189-1744, or 928-1268 of SEQ ID NO:76; not include all of the nucleotides of GenBank™ accession number BE736451, BE792564, AW239215, AF159442, or AF097738, e.g., can be one or more nucleotides shorter (at one or both ends) than the sequence of GenBank™ accession number BE736451, BE792564, AW239215, AF159442, or AF097738; or can differ by one or more nucleotides in the region of overlap.

57779 Nucleic Acid Fragments

A nucleic acid molecule of the invention can include only a portion of the nucleic acid sequence of SEQ ID NO:79 or SEQ ID NO:81. For example, such a nucleic acid molecule can include a fragment which can be used as a probe or primer or a fragment encoding a portion of a 57779 protein, e.g., an immunogenic or biologically active portion of a 57779 protein. A fragment can comprise those nucleotides of SEQ ID NO:79, which encode at least one cadherin domains of human 57779. The nucleotide sequence determined from the cloning of the 57779 gene allows for the generation of probes and primers designed for use in identifying and/or cloning other 57779 family members, or fragments thereof, as well as 57779 homologues, or fragments thereof, from other species.

In another embodiment, a nucleic acid includes a nucleotide sequence that includes part, or all, of the coding region and extends into either (or both) the 5′ or 3′ noncoding region. Other embodiments include a fragment that includes a nucleotide sequence encoding an amino acid fragment described herein. Nucleic acid fragments can encode a specific domain or site described herein or fragments thereof, particularly fragments thereof which are at least 100, 200, 300, 400, 500, or 750 amino acids in length. Preferably, the nucleic acid fragments encode a specific domain or fragment thereof, wherein the domain or fragment is at least 400 amino acids in length, e.g., the domain can be a protocadherin domain that includes six cadherin domains. Fragments also include nucleic acid sequences corresponding to specific amino acid sequences described above or fragments thereof. Nucleic acid fragments should not to be construed as encompassing those fragments that may have been disclosed prior to the invention.

A nucleic acid fragment can include a sequence corresponding to a domain, region, or functional site described herein. A nucleic acid fragment can also include one or more domain, region, or functional site described herein. Thus, for example, a 57779 nucleic acid fragment can include a sequence corresponding to a cadherin domain, e.g., one, two, three, four or more cadherin domains, e.g., a protocadherin domain having six cadherin domains.

57779 probes and primers are provided. Typically a probe/primer is an isolated or purified oligonucleotide. The oligonucleotide typically includes a region of nucleotide sequence that hybridizes under a stringency condition described herein to at least about 7, 12 or 15, preferably about 20 or 25, more preferably about 30, 35, 40, 45, 50, 55, 60, 65, or 75 consecutive nucleotides of a sense or antisense sequence of SEQ ID NO:79 or SEQ ID NO:81, or of a naturally occurring allelic variant or mutant of SEQ ID NO:79 or SEQ ID NO:81. Preferably, an oligonucleotide is less than about 200, 150, 120, or 100 nucleotides in length.

In one embodiment, the probe or primer, is attached to a solid support, e.g., a solid support described herein.

One exemplary kit of primers includes a forward primer that anneals to the coding strand and a reverse primer that anneals to the non-coding strand. The forward primer can anneal to the start codon, e.g., the nucleic acid sequence encoding amino acid residue 1 of SEQ ID NO:80. The reverse primer can anneal to the ultimate codon, e.g., the codon immediately before the stop codon, e.g., the codon encoding amino acid residue 873 of SEQ ID NO:80. In a preferred embodiment, the annealing temperatures of the forward and reverse primers differ by no more than 5, 4, 3, or 2° C.

In a preferred embodiment the nucleic acid is a probe which is at least 10, 12, 15, 18, 20 and less than 200, more preferably less than 100, or less than 50, nucleotides in length. It should be identical, or differ by 1, or 2, or less than 5 or 10 nucleotides, from a sequence disclosed herein. If alignment is needed for this comparison the sequences should be aligned for maximum homology. “Looped” out sequences from deletions or insertions, or mismatches, are considered differences.

A probe or primer can be derived from the sense or anti-sense strand of a nucleic acid which encodes: a cadherin domain of 57779, e.g., located at about 25 to 120, 134 to 229, 243 to 337, 354 to 444, 458 to 554, and 573 to 663 of SEQ ID NO:80; a signal peptide of 57779, e.g., located at about amino acids 8 to 25 of SEQ ID NO:80; a transmembrane domain of 57779, e.g., located at about amino acids 677 to 701, 835 to 859 of SEQ ID NO:80; an extracellular domain of 57779, e.g., located at about 26 to 676, and about 860 to 873 of SEQ ID NO:80; or an intracellular domain of 57779, e.g., located at about 702 to 834 of SEQ ID NO:80.

In another embodiment a set of primers is provided, e.g., primers suitable for use in a PCR, which can be used to amplify a selected region of a 57779 sequence, e.g., a domain, region, site or other sequence described herein. The primers should be at least 5, 10, or 50 base pairs in length and less than 100, or less than 200, base pairs in length. The primers should be identical, or differs by one base from a sequence disclosed herein or from a naturally occurring variant. For example, primers suitable for amplifying all or a portion of any of the following regions are provided: a protocadherin domain from about amino acid 25 to 663 of SEQ ID NO:80; a cadherin domain of 57779, e.g., located at about 25 to 120, 134 to 229, 243 to 337, 354 to 444, 458 to 554, and 573 to 663 of SEQ ID NO:80; a signal peptide of 57779, e.g., located at about amino acids 8 to 25 of SEQ ID NO:80; a transmembrane domain of 57779, e.g., located at about amino acids 677 to 701, 835 to 859 of SEQ ID NO:80; an extracellular domain of 57779, e.g., located at about 26 to 676, and about 860 to 873 of SEQ ID NO:80; or an intracellular domain of 57779, e.g., located at about 702 to 834 of SEQ ID NO:80.

A nucleic acid fragment can encode an epitope bearing region of a polypeptide described herein.

A nucleic acid fragment encoding a “biologically active portion of a 57779 polypeptide” can be prepared by isolating a portion of the nucleotide sequence of SEQ ID NO:79 or SEQ ID NO:81, which encodes a polypeptide having a 57779 biological activity (e.g., the biological activities of the 57779 proteins are described herein), expressing the encoded portion of the 57779 protein (e.g., by recombinant expression in vitro) and assessing the activity of the encoded portion of the 57779 protein. For example, a nucleic acid fragment encoding a biologically active portion of 57779 includes a protocadherin domain (e.g., amino acid residues about 25 to 663 of SEQ ID NO:80), a cadherin domain of 57779 (e.g., located at about 25 to 120, 134 to 229, 243 to 337, 354 to 444, 458 to 554, and 573 to 663 of SEQ ID NO:80), a signal peptide of 57779 (e.g., located at about amino acids 8 to 25 of SEQ ID NO:80); a transmembrane domain of 57779 (e.g., located at about amino acids 677 to 701, 835 to 859 of SEQ ID NO:80), an extracellular domain of 57779 (e.g., located at about 26 to 676, and about 860 to 873 of SEQ ID NO:80), or an intracellular domain of 57779 (e.g., located at about 702 to 834 of SEQ ID NO:80). A nucleic acid fragment encoding a biologically active portion of a 57779 polypeptide, may comprise a nucleotide sequence which is greater than 300, 500, 800, 1500, 2000, or more nucleotides in length.

In preferred embodiments, a nucleic acid includes a nucleotide sequence which is about 300, 400, 500, 600, 700, 800, 900, 1000, 1100, 1200, 1300, 1400, 1500, 1600, 1700, 1800, 1900, 2000, 2200, 2400, 2600, or more nucleotides in length and hybridizes under a stringency condition described herein to a nucleic acid molecule of SEQ ID NO:79, or SEQ ID NO:81.

In a preferred embodiment, a nucleic acid fragment differs by at least 1, 2, 3, 10, 20, or more nucleotides from a 57779 variant sequence. Differences can include differing in length or sequence identity. For example, a nucleic acid fragment can: include one or more nucleotides from SEQ ID NO:79 or SEQ ID NO:81 located outside the region of nucleotides 1984 to 2181 or 1780 to 2366. The nucleic acid fragment can include a sequence identical to the region of nucleotides 1-300, 300-800, 500-1000, 700-1300, 1000-1500, or 1400-1900 of SEQ ID NO:79.

23566, 33489, or 57779 Nucleic Acid Variants

The invention further encompasses nucleic acid molecules that differ from the nucleotide sequence shown in SEQ ID NO:73, SEQ ID NO:76, SEQ ID NO:79, SEQ ID NO:75, SEQ ID NO:78, or SEQ ID NO:81. Such differences can be due to degeneracy of the genetic code (and result in a nucleic acid which encodes the same 23566, 33489, or 57779 proteins as those encoded by the nucleotide sequence disclosed herein. In another embodiment, an isolated nucleic acid molecule of the invention has a nucleotide sequence encoding a protein having an amino acid sequence which differs, by at least 1, but less than 5, 10, 20, 50, or 100 amino acid residues that shown in SEQ ID NO:74, SEQ ID NO:77, or SEQ ID NO:80. If alignment is needed for this comparison the sequences should be aligned for maximum homology. The encoded protein can differ by no more than 5, 4, 3, 2, or 1 amino acid. “Looped” out sequences from deletions or insertions, or mismatches, are considered differences.

Nucleic acids of the inventor can be chosen for having codons, which are preferred, or non-preferred, for a particular expression system. E.g., the nucleic acid can be one in which at least one codon, at preferably at least 10%, or 20% of the codons has been altered such that the sequence is optimized for expression in E. coli, yeast, human, insect, or CHO cells.

Nucleic acid variants can be naturally occurring, such as allelic variants (same locus), homologs (different locus), and orthologs (different organism) or can be non naturally occurring. Non-naturally occurring variants can be made by mutagenesis techniques, including those applied to polynucleotides, cells, or organisms. The variants can contain nucleotide substitutions, deletions, inversions and insertions. Variation can occur in either or both the coding and non-coding regions. The variations can produce both conservative and non-conservative amino acid substitutions (as compared in the encoded product).

In a preferred embodiment, the nucleic acid differs from that of SEQ ID NO:73, SEQ ID NO:75, SEQ ID NO:76, SEQ ID NO:78, SEQ ID NO:79, or SEQ ID NO:81, e.g., as follows: by at least one but less than 10, 20, 30, or 40 nucleotides; at least one but less than 1%, 5%, 10% or 20% of the nucleotides in the subject nucleic acid. The nucleic acid can differ by no more than 5, 4, 3, 2, or 1 nucleotide. If necessary for this analysis the sequences should be aligned for maximum homology. “Looped” out sequences from deletions or insertions, or mismatches, are considered differences.

Orthologs, homologs, and allelic variants can be identified using methods known in the art. These variants comprise a nucleotide sequence encoding a polypeptide that is 50%, at least about 55%, typically at least about 70-75%, more typically at least about 80-85%, and most typically at least about 90-95% or more identical to the nucleotide sequences that encode the sequences shown in SEQ ID NO:74, SEQ ID NO:77, or SEQ ID NO:80, or a fragment thereof. Such nucleic acid molecules can readily be identified as being able to hybridize under a stringency condition described herein, to the nucleotide sequence that encodes a sequence shown in SEQ ID NO:74, SEQ ID NO:77, or SEQ ID NO:80, or a fragment thereof. Nucleic acid molecules corresponding to orthologs, homologs, and allelic variants of the 23566, 33489, or 57779 cDNAs of the invention can further be isolated by mapping to the same chromosome or locus as the 23566, 33489, or 57779 gene.

Preferred variants include those that are correlated with peptide hydrolysis activity (e.g., carboxy terminal peptide hydrolysis activity), scramblase activity, or cellular adhesive activity (e.g., homotypic cell-cell adhesion).

Allelic variants of 23566, e.g., human 23566, include both functional and non-functional proteins. Functional allelic variants are naturally occurring amino acid sequence variants of the 23566 protein within a population that maintain the ability to bind and hydrolyze polypeptide substrates. Functional allelic variants will typically contain only conservative substitution of one or more amino acids of SEQ ID NO:74, or substitution, deletion or insertion of non-critical residues in non-critical regions of the protein. Non-functional allelic variants are naturally-occurring amino acid sequence variants of the 23566, e.g., human 23566, protein within a population that do not have the ability to bind and/or hydrolyze peptide substrates. Non-functional allelic variants will typically contain a non-conservative substitution, a deletion, or insertion, or premature truncation of the amino acid sequence of SEQ ID NO:74, or a substitution, insertion, or deletion in critical residues or critical regions of the protein.

Allelic variants of 33489, e.g., human 33489, include both functional and non-functional proteins. Functional allelic variants are naturally occurring amino acid sequence variants of the 33489 protein within a population that maintain the ability to catalyze the redistribution of phospholipids between leaflets of the lipid bilayer. Functional allelic variants will typically contain only conservative substitution of one or more amino acids of SEQ ID NO:77, or substitution, deletion or insertion of non-critical residues in non-critical regions of the protein. Non-functional allelic variants are naturally-occurring amino acid sequence variants of the 33489, e.g., human 33489, protein within a population that do not have the ability to catalyze the redistribution of phospholipids between leaflets of the lipid bilayer. Non-functional allelic variants will typically contain a non-conservative substitution, a deletion, or insertion, or premature truncation of the amino acid sequence of SEQ ID NO:77, or a substitution, insertion, or deletion in critical residues or critical regions of the protein.

Allelic variants of 57779, e.g., human 57779, include both functional and non-functional proteins. Functional allelic variants are naturally occurring amino acid sequence variants of the 57779 protein within a population that maintain the ability to mediate intermolecular association at the cell surface. Functional allelic variants will typically contain only conservative substitution of one or more amino acids of SEQ ID NO:80, or substitution, deletion or insertion of non-critical residues in non-critical regions of the protein. Non-functional allelic variants are naturally-occurring amino acid sequence variants of the 57779, e.g., human 57779, protein within a population that do not have the ability to mediate intermolecular association at the cell surface. Non-functional allelic variants will typically contain a non-conservative substitution, a deletion, or insertion, or premature truncation of the amino acid sequence of SEQ ID NO:80, or a substitution, insertion, or deletion in critical residues or critical regions of the protein.

Moreover, nucleic acid molecules encoding other 23566, 33489, or 57779 family members and, thus, which have a nucleotide sequence which differs from the 23566, 33489, or 57779 sequences of SEQ ID NO:73, SEQ ID NO:76, SEQ ID NO:79, SEQ ID NO:75, SEQ ID NO:78, or SEQ ID NO:81 are intended to be within the scope of the invention.

Antisense Nucleic Acid Molecules, Ribozymes and Modified 23566, 33489, or 57779 Nucleic Acid Molecules

In another aspect, the invention features, an isolated nucleic acid molecule which is antisense to 23566, 33489, or 57779. An “antisense” nucleic acid can include a nucleotide sequence which is complementary to a “sense” nucleic acid encoding a protein, e.g., complementary to the coding strand of a double-stranded cDNA molecule or complementary to an mRNA sequence. The antisense nucleic acid can be complementary to an entire 23566, 33489, or 57779 coding strand, or to only a portion thereof (e.g., the coding region of human 23566, 33489, or 57779 corresponding to SEQ ID NO:75, SEQ ID NO:78, and SEQ ID NO:81, respectively). In another embodiment, the antisense nucleic acid molecule is antisense to a “noncoding region” of the coding strand of a nucleotide sequence encoding 23566, 33489, or 57779 (e.g., the 5′ and 3′ untranslated regions).

An antisense nucleic acid can be designed such that it is complementary to the entire coding region of 23566, 33489, or 57779 mRNA, but more preferably is an oligonucleotide which is antisense to only a portion of the coding or noncoding region of 23566, 33489, or 57779 mRNA. For example, the antisense oligonucleotide can be complementary to the region surrounding the translation start site of 23566, 33489, or 57779 mRNA, e.g., between the −10 and +10 regions of the target gene nucleotide sequence of interest. An antisense oligonucleotide can be, for example, about 7, 10, 15, 20, 25, 30, 35, 40, 45, 50, 55, 60, 65, 70, 75, 80, or more nucleotides in length.

An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. The antisense nucleic acid also can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject (e.g., by direct injection at a tissue site), or generated in situ such that they hybridize with or bind to cellular mRNA and/or genomic DNA encoding a 23566, 33489, or 57779 protein to thereby inhibit expression of the protein, e.g., by inhibiting transcription and/or translation. Alternatively, antisense nucleic acid molecules can be modified to target selected cells and then administered systemically. For systemic administration, antisense molecules can be modified such that they specifically bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids. Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

In still another embodiment, an antisense nucleic acid of the invention is a ribozyme. A ribozyme having specificity for a 23566, 33489, or 57779-encoding nucleic acid can include one or more sequences complementary to the nucleotide sequence of a 23566, 33489, or 57779 cDNA disclosed herein (i.e., SEQ ID NO:73, SEQ ID NO:76, SEQ ID NO:79, SEQ ID NO:75, SEQ ID NO:78, or SEQ ID NO:81), and a sequence having known catalytic sequence responsible for mRNA cleavage (see U.S. Pat. No. 5,093,246 or Haselhoff and Gerlach (1988) Nature 334:585-591). For example, a derivative of a Tetrahymena L-19 IVS RNA can be constructed in which the nucleotide sequence of the active site is complementary to the nucleotide sequence to be cleaved in a 23566, 33489, or 57779-encoding mRNA. See, e.g., Cech et al. U.S. Pat. No. 4,987,071; and Cech et al. U.S. Pat. No. 5,116,742. Alternatively, 23566, 33489, or 57779 mRNA can be used to select a catalytic RNA having a specific ribonuclease activity from a pool of RNA molecules. See, e.g., Bartel, D. and Szostak, J. W. (1993) Science 261:1411-1418.

23566, 33489, or 57779 gene expression can be inhibited by targeting nucleotide sequences complementary to the regulatory region of the 23566, 33489, or 57779 (e.g., the 23566, 33489, or 57779 promoter and/or enhancers) to form triple helical structures that prevent transcription of the 23566, 33489, or 57779 gene in target cells. See generally, Helene, C. (1991) Anticancer Drug Des. 6:569-84; Helene, C. i (1992) Ann. N.Y. Acad. Sci. 660:27-36; and Maher, L. J. (1992) Bioassays 14:807-15. The potential sequences that can be targeted for triple helix formation can be increased by creating a so-called “switchback” nucleic acid molecule. Switchback molecules are synthesized in an alternating 5′-3′, 3′-5′ manner, such that they base pair with first one strand of a duplex and then the other, eliminating the necessity for a sizeable stretch of either purines or pyrimidines to be present on one strand of a duplex.

The invention also provides detectably labeled oligonucleotide primer and probe molecules. Typically, such labels are chemiluminescent, fluorescent, radioactive, or colorimetric.

A 23566, 33489, or 57779 nucleic acid molecule can be modified at the base moiety, sugar moiety or phosphate backbone to improve, e.g., the stability, hybridization, or solubility of the molecule. For non-limiting examples of synthetic oligonucleotides with modifications see Toulmé (2001) Nature Biotech. 19:17 and Faria et al. (2001) Nature Biotech. 19:40-44. Such phosphoramidite oligonucleotides can be effective antisense agents.

For example, the deoxyribose phosphate backbone of the nucleic acid molecules can be modified to generate peptide nucleic acids (see Hyrup B. et al. (1996) Bioorganic & Medicinal Chemistry 4: 5-23). As used herein, the terms “peptide nucleic acid” or “PNA” refers to a nucleic acid mimic, e.g., a DNA mimic, in which the deoxyribose phosphate backbone is replaced by a pseudopeptide backbone and only the four natural nucleobases are retained. The neutral backbone of a PNA can allow for specific hybridization to DNA and RNA under conditions of low ionic strength. The synthesis of PNA oligomers can be performed using standard solid phase peptide synthesis protocols as described in Hyrup B. et al. (1996) supra and Perry-O'Keefe et al. Proc. Natl. Acad. Sci. 93: 14670-675.

PNAs of 23566, 33489, or 57779 nucleic acid molecules can be used in therapeutic and diagnostic applications. For example, PNAs can be used as antisense or antigene agents for sequence-specific modulation of gene expression by, for example, inducing transcription or translation arrest or inhibiting replication. PNAs of 23566, 33489, or 57779 nucleic acid molecules can also be used in the analysis of single base pair mutations in a gene, (e.g., by PNA-directed PCR clamping); as ‘artificial restriction enzymes’ when used in combination with other enzymes, (e.g., S1 nucleases (Hyrup B. et al. (1996) supra)); or as probes or primers for DNA sequencing or hybridization (Hyrup B. et al. (1996) supra; Perry-O'Keefe supra).

In other embodiments, the oligonucleotide may include other appended groups such as peptides (e.g., for targeting host cell receptors in vivo), or agents facilitating transport across the cell membrane (see, e.g., Letsinger et al. (1989) Proc. Natl. Acad. Sci. USA 86:6553-6556; Lemaitre et al. (1987) Proc. Natl. Acad. Sci. USA 84:648-652; PCT Publication No. WO88/09810) or the blood-brain barrier (see, e.g., PCT Publication No. WO89/10134). In addition, oligonucleotides can be modified with hybridization-triggered cleavage agents (see, e.g., Krol et al. (1988) Bio-Techniques 6:958-976) or intercalating agents. (see, e.g., Zon (1988) Pharm. Res. 5:539-549). To this end, the oligonucleotide may be conjugated to another molecule, (e.g., a peptide, hybridization triggered cross-linking agent, transport agent, or hybridization-triggered cleavage agent).

The invention also includes molecular beacon oligonucleotide primer and probe molecules having at least one region which is complementary to a 23566, 33489, or 57779 nucleic acid of the invention, two complementary regions one having a fluorophore and one a quencher such that the molecular beacon is useful for quantitating the presence of the 23566, 33489, or 57779 nucleic acid of the invention in a sample. Molecular beacon nucleic acids are described, for example, in Lizardi et al., U.S. Pat. No. 5,854,033; Nazarenko et al., U.S. Pat. No. 5,866,336, and Livak et al., U.S. Pat. No. 5,876,930.

Isolated 23566, 33489, or 57779 Polypeptides

In another aspect, the invention features, an isolated 23566, 33489, or 57779 protein, or fragment, e.g., a biologic ally active portion, for use as immunogens or antigens to raise or test (or more generally to bind) anti-23566, 33489, or 57779 antibodies. 23566, 33489, or 57779 protein can be isolated from cells or tissue sources using standard protein purification techniques. 23566, 33489, or 57779 protein or fragments thereof can be produced by recombinant DNA techniques or synthesized chemically.

Polypeptides of the invention include those which arise as a result of the existence of multiple genes, alternative transcription events, alternative RNA splicing events, and alternative translational and post-translational events. The polypeptide can be expressed in systems, e.g., cultured cells, which result in substantially the same post-translational modifications present when expressed the polypeptide is expressed in a native cell, or in systems which result in the alteration or omission of post-translational modifications, e.g., glycosylation or cleavage, present when expressed in a native cell.

In a preferred embodiment, a 23566 polypeptide has one or more of the following characteristics:

(i) it has the ability to bind to and hydrolyze polypeptide substrates, e.g., to hydrolyze the C-terminal residue of a polypeptide substrate;

(ii) it has a molecular weight, e.g., a deduced molecular weight, preferably ignoring any contribution of post translational modifications, amino acid composition or other physical characteristic of a 23566 polypeptide, e.g., a polypeptide of SEQ ID NO:74;

(iii) it has an overall sequence similarity of at least 60%, more preferably at least 70%, 80%, 90%, 95%, 98%, 99%, or more with a polypeptide a of SEQ ID NO:74;

(iv) it can be found in the pancreas, the circulation, or the brain;

(v) it has at least one zinc carboxypeptidase domain which is preferably about 70%, 80%, 90% or 95% with amino acid residues about 180 to 443 of SEQ ID NO:74;

(vi) it has at least one zinc carboxypeptidase zinc-binding region 1 signature motif (PS00132);

(vii) it has at least one predicted signal peptide;

(viii) it has at least one, two, preferably three predicted N-glycosylation sites;

(ix) it has at least one, two, three, four, preferably five predicted Protein Kinase C phosphorylation sites (PS00005);

(x) it has at least one, two, three, four, preferably five predicted Casein Kinase II phosphorylation sites (PS00006);

(xi) is has at least one predicted Tyrosine kinase phosphorylation site (PS00007) located at about amino acid residues 132 to 139 of SEQ ID NO:74;

(xii) it has at least one, two, three, four, preferably five predicted N-myristylation sites (PS00008);

(xiii) it has at least one predicted Amidation site (PS00009); or

(vi) it has at least 5, preferably 10, and most preferably 13 of the cysteines found amino acid sequence of the native protein.

In a preferred embodiment the 23566 protein, or fragment thereof, differs from the corresponding sequence in SEQ ID:2. In one embodiment it differs by at least one but by less than 15, 10 or 5 amino acid residues. In another it differs from the corresponding sequence in SEQ ID NO:74 by at least one residue but less than 20%, 15%, 10% or 5% of the residues in it differ from the corresponding sequence in SEQ ID NO:74. (If this comparison requires alignment the sequences should be aligned for maximum homology. “Looped” out sequences from deletions or insertions, or mismatches, are considered differences.) The differences are, preferably, differences or changes at a non-essential residue or a conservative substitution. In a preferred embodiment the differences are not in the zinc carboxypeptidase domain, e.g., from about amino acid 180 to 443 of SEQ ID NO:74. In another preferred embodiment one or more differences are in the zinc carboxypeptidase domain, e.g., from about amino acid 180 to 443 of SEQ ID NO:74.

Other embodiments include a protein that contain one or more changes in amino acid sequence, e.g., a change in an amino acid residue which is not essential for activity. Such 23566 proteins differ in amino acid sequence from SEQ ID NO:74, yet retain biological activity.

In one embodiment, the protein includes an amino acid sequence at least about 60%, 65%, 70%, 75%, 80%, 85%, 90%, 95%, 98% or more homologous to SEQ ID NO:74.

A 23566 protein or fragment is provided which varies from the sequence of SEQ ID NO:74 in regions defined by amino acids about 1 to 179 by at least one but by less than 15, 10 or 5 amino acid residues in the protein or fragment but which does not differ from SEQ ID NO:74 in regions defined by amino acids about 180 to 443. (If this comparison requires alignment the sequences should be aligned for maximum homology. “Looped” out sequences from deletions or insertions, or mismatches, are considered differences.) In some embodiments the difference is at a non-essential residue or is a conservative substitution, while in others the difference is at an essential residue or is a non-conservative substitution.

In one embodiment, a biologically active portion of a 23566 protein includes a zinc carboxypeptidase domain. Moreover, other biologically active portions, in which other regions of the protein are deleted, can be prepared by recombinant techniques and evaluated for one or more of the functional activities of a native 23566 protein.

In a preferred embodiment, the 23566 protein has an amino acid sequence shown in SEQ ID NO:74. In other embodiments, the 23566 protein is substantially identical to SEQ ID NO:74. In yet another embodiment, the 23566 protein is substantially identical to SEQ ID NO:74 and retains the functional activity of the protein of SEQ ID NO:74, as described in detail in the subsections above.

In a preferred embodiment, a fragment differs by at least 1, 2, 3, 10, 20, or more amino acid residues from a protein encoded by a sequence present in Genbank™ accession number AX083139, F81728, X51497 or C00454, or SEQ ID NOs:1, 2, 3, 5, 23, or 26 of WO2001157165. Differences can include differing in length or sequence identity. For example, a fragment can: include one or more amino acid residues from SEQ ID NO:74 outside the region encoded by nucleotides 1 to 322, 597 to 1602, 133 to 143, or 589 to 913 of SEQ ID NO:73; not include all of the amino acid residues encoded by a nucleotide sequence present in Genbank™ accession number AX083139, F81728, X51497 or C00454, or SEQ ID NOs:1, 2, 3, 5, 23, or 26 of WO2001157165, e.g., can be one or more amino acid residues shorter (at one or both ends) than a sequence encoded by the nucleotide sequence present in Genbank™ accession number AX083139, F81728, X51497 or C00454, or SEQ ID NOs:1, 2, 3, 5, 23, or 26 of WO2001157165; or can differ by one or more amino acid residues in the region of overlap.

In a preferred embodiment, a 33489 polypeptide has one or more of the following characteristics:

(i) it has scramblase activity, i.e., the ability to catalyze the redistribution of phospholipids between leaflets of the lipid bilayer;

(ii) it has a molecular weight, e.g., a deduced molecular weight, preferably ignoring any contribution of post translational modifications, amino acid composition or other physical characteristic of a 33489 polypeptide, e.g., a polypeptide of SEQ ID NO:74;

(iii) it has an overall sequence similarity of at least 60%, more preferably at least 70%, 80%, 90%, 95%, 98%, 99%, or more with a polypeptide of SEQ ID NO:77;

(iv) it is an integral membrane protein;

(v) it is expressed in coronary smooth muscle cells and endothelial cells;

(vi) it has at least one scramblase domain which is preferably at least about 70%, 80%, 90% or 95% identical to amino acid residues about 103 to 285 of SEQ ID NO:77, including at least one, two, three, four, preferably five conserved functional side chains that coorespond to the amino acid residues locates at about 250, 252, 254, 258, and 261 of SEQ ID NO:77;

(vii) it has type II transmembrane topology including one predicted amino terminal cytoplasmic domain, one predicted transmembrane domain, and one predicted carboxy terminal extracellular domain;

(viii) it has has at least one proline rich potential SH3-domain binding motif;

(ix) it has at least one, two, three, four, preferably five predicted casein kinase II phosphorylation sites (PS00006); and

(x) it has at least one, two, three, four, preferably five predicted N-myristylation sites (PS00008).

In a preferred embodiment the 33489 protein, or fragment thereof, differs from the corresponding sequence in SEQ ID:5. In one embodiment it differs by at least one but by less than 15, 10 or 5 amino acid residues. In another embodiment, it differs from the corresponding sequence in SEQ ID NO:77 by at least one residue, but less than 20%, 15%, 10% or 5% of the residues in it differ from the corresponding sequence in SEQ ID NO:77. (If this comparison requires alignment the sequences should be aligned for maximum homology. “Looped” out sequences from deletions or insertions, or mismatches, are considered differences.) The differences are, preferably, differences or changes at a non-essential residue or a conservative substitution. In a preferred embodiment, the differences are not in the scramblase domain, e.g., about amino acid residues 103 to 285 of SEQ If) NO:77. In another preferred embodiment, one or more differences are in the scramblase domain, e.g., about amino acid residues 103 to 285 of SEQ ID NO:77.

Other embodiments include a protein that contain one or more changes in amino acid sequence, e.g., a change in an amino acid residue which is not essential for activity. Such 33489 proteins differ in amino acid sequence from SEQ ID NO:77, yet retain biological activity.

In one embodiment, the protein includes an amino acid sequence at least about 60%, 65%, 70%, 75%, 80%, 85%, 90%, 95%, 98% or more homologous to SEQ ID NO:77.

A 33489 protein or fragment is provided which varies from the sequence of SEQ ID NO:77 in regions defined by amino acids about 1 to 106 and 286 to 296 of SEQ ID NO:77, by at least one but by less than 15, 10 or 5 amino acid residues in the protein or fragment, but which does not differ from SEQ ID NO:77 in regions defined by amino acids about 103 to 285 of SEQ ID NO:77. (If this comparison requires alignment the sequences should be aligned for maximum homology. “Looped” out sequences from deletions or insertions, or mismatches, are considered differences.) In some embodiments the difference is at a non-essential residue or is a conservative substitution, while in others the difference is at an essential residue or is a non-conservative substitution.

In one embodiment, a biologically active portion of a 33489 protein includes a scramblase domain. Moreover, other biologically active portions, in which other regions of the protein are deleted, can be prepared by recombinant techniques and evaluated for one or more of the functional activities of a native 33489 protein.

In a preferred embodiment, the 33489 protein has an amino acid sequence shown in SEQ ID NO:77. In other embodiments, the 33489 protein is substantially identical to SEQ ID NO:77. In yet another embodiment, the 33489 protein is substantially identical to SEQ ID NO:77 and retains the functional activity of the protein of SEQ ID NO:77, as described in detail in the subsections above.

In a preferred embodiment, a fragment differs by at least 1, 2, 3, 10, 20, or more amino acid residues encoded by a sequence present in GenBank™ accession number BE736451, BE792564, AW239215, AF159442, or AF097738. Differences can include differing in length or sequence identity. For example, a fragment can: include one or more amino acid residues from SEQ ID NO:77 outside the region encoded by nucleotides 433-892, 436-1030, 41-658, 1189-1744, or 928-1268 of SEQ ID NO:76; not include all of the amino acid residues encoded by a nucleotide sequence in GenBank™ accession number BE736451, BE792564, AW239215, AF159442, or AF097738, e.g., can be one or more amino acid residues shorter (at one or both ends) than a sequence encoded by the nucleotide sequence in GenBank™ accession number BE736451, BE792564, AW239215, AF159442, or AF097738; or can differ by one or more amino acid residues in the region of overlap.

In a preferred embodiment, a 57779 polypeptide has one or more of the following characteristics:

(i) it has the ability to mediate cell-cell adhesion, e.g., homotypic cell-cell adhesion;

(ii) it has a molecular weight, e.g., a deduced molecular weight, preferably ignoring any contribution of post translational modifications, amino acid composition or other physical characteristic of a 57779 polypeptide, e.g., a polypeptide of SEQ ID NO:80;

(iii) it has an overall sequence similarity of at least 60%, more preferably at least 70%, 80%, 90%, 95%, 98%, 99%, or more with a polypeptide of SEQ ID NO:80;

(iv) it can be found on the cell surface as an integral membrane protein, e.g., in a mature form;

(v) it has at least one, two, three, four, five, preferably six cadherin domains which preferably share about 70%, 80%, 90%, 95%, 98%, 99%, or more homology with at least one cadherin domain of human 57779, e.g., about amino acid residues about 25 to 120, 134 to 229, 243 to 337, 354 to 444, 458 to 554, or 573 to 663 of SEQ ID NO:80;

(vi) it has at least one, two, three, four, preferably five cadherin extracellular domain signature motifs (PS00232);

(vii) it has a signal peptide;

(viii) it has at least one, preferably two predicted transmembrane domains;

(ix) it has at least one, preferably two predicted extracellular domains, e.g., an N-terminal extracellular domain and a C-terminal extracellular domain;

(x) it has at least one, two, three, four, five, six, seven, eight, nine, ten, preferably eleven predicted protein kinase C phosphorylation sites (PS00005);

(xi) it has at least one, two, three, four, five, six, seven, eight, nine, ten, eleven, twelve, thirteen, fourteen, preferably fifteen predicted casein kinase II phosphorylation sites (PS00006);

(xii) it has at least one, two, preferably three predicted tyrosine kinase phosphorylation sites (PS00007);

(xiii) it has at least one predicted RGD cell attachment sequence (PS00016);

(xiv) it has at least one predicted glycosaminoglycan attachment site (PS00002);

(xii) it has at least one, two, three, four, five, six, preferably seven predicted N-glycosylation sites (PS00001); and

(xiii) it has at least one, two, three, four, five, six, preferably seven predicted N-myristylation sites (PS00008).

In a preferred embodiment the 57779 protein, or fragment thereof, differs from the corresponding sequence in SEQ ID:8. In one embodiment it differs by at least one but by less than 15, 10 or 5 amino acid residues. In another it differs from the corresponding sequence in SEQ ID NO:80 by at least one residue but less than 20%, 15%, 10% or 5% of the residues in it differ from the corresponding sequence in SEQ ID NO:80. (If this comparison requires alignment the sequences should be aligned for maximum homology. “Looped” out sequences from deletions or insertions, or mismatches, are considered differences.) The differences are, preferably, differences or changes at a non-essential residue or a conservative substitution. In a preferred embodiment the differences are not in the protocadherin domain, e.g., about amino acid residues 25 to 663 of SEQ ID NO:80. In another preferred embodiment one or more differences are in the protocadherin domain, e.g., about amino acid residues 25 to 663 of SEQ ID NO:80.

Other embodiments include a protein that contain one or more changes in amino acid sequence, e.g., a change in an amino acid residue which is not essential for activity. Such 57779 proteins differ in amino acid sequence from SEQ ID NO:80, yet retain biological activity.

In one embodiment, the protein includes an amino acid sequence at least about 60%, 65%, 70%, 75%, 80%, 85%, 90%, 95%, 98% or more homologous to SEQ ID NO:80.

A 57779 protein or fragment is provided which varies from the sequence of SEQ ID NO:80 in regions defined by amino acids about 25 to 663 by at least one but by less than 15, 10 or 5 amino acid residues in the protein or fragment but which does not differ from SEQ ID NO:80 in regions defined by amino acids about 25 to 663. (If this comparison requires alignment the sequences should be aligned for maximum homology. “Looped” out sequences from deletions or insertions, or mismatches, are considered differences.) In some embodiments the difference is at a non-essential residue or is a conservative substitution, while in others the difference is at an essential residue or is a non-conservative substitution.

In one embodiment, a biologically active portion of a 57779 protein includes at least one cadherin domain, all six cadherin domains, an N-terminal extracellular domain, a C-terminal extracellular domain, a transmembrane domain, a cytoplasmic loop, or a signal sequence. Moreover, other biologically active portions, in which other regions of the protein are deleted, can be prepared by recombinant techniques and evaluated for one or more of the functional activities of a native 57779 protein.

In a preferred embodiment, the 57779 protein has an amino acid sequence shown in SEQ ID NO:80. In other embodiments, the 57779 protein is substantially identical to SEQ ID NO:80. In yet another embodiment, the 57779 protein is substantially identical to SEQ ID NO:80 and retains the functional activity of the protein of SEQ ID NO:80, as described in detail in the subsections above.

In a preferred embodiment, a fragment differs by at least 1, 2, 3, 10, 20, or more amino acid residues encoded by a 57779 variant sequence. Differences can include differing in length or sequence identity. In another embodiment, a 57779 protein fragment includes one or more cadherin domains and is at least about 100, 200, 300, 400, 500, or 600 amino acids in length.

23566, 33489, or 57779 Chimeric or Fusion Proteins

In another aspect, the invention provides 23566, 33489, or 57779 chimeric or fusion proteins. As used herein, a 23566, 33489, or 57779 “chimeric protein” or “fusion protein” includes a 23566, 33489, or 57779 polypeptide linked to a non-23566, 33489, or 57779 polypeptide. A “non-23566, 33489, or 57779 polypeptide” refers to a polypeptide having an amino acid sequence corresponding to a protein which is not substantially homologous to the 23566, 33489, or 57779 protein, e.g., a protein which is different from the 23566, 33489, or 57779 protein and which is derived from the same or a different organism. The 23566, 33489, or 57779 polypeptide of the fusion protein can correspond to all or a portion e.g., a fragment described herein of a 23566, 33489, or 57779 amino acid sequence. In a preferred embodiment, a 23566, 33489, or 57779 fusion protein includes at least one (or two) biologically active portion of a 23566, 33489, or 57779 protein. The non-23566, 33489, or 57779 polypeptide can be fused to the N-terminus or C-terminus of the 23566, 33489, or 57779 polypeptide.

The fusion protein can include a moiety which has a high affinity for a ligand. For example, the fusion protein can be a GST-23566, 33489, or 57779 fusion protein in which the 23566, 33489, or 57779 sequences are fused to the C-terminus of the GST sequences. Such fusion proteins can facilitate the purification of recombinant 23566, 33489, or 57779. Alternatively, the fusion protein can be a 23566, 33489, or 57779 protein containing a heterologous signal sequence at its N-terminus. In certain host cells (e.g., mammalian host cells), expression and/or secretion of 23566, 33489, or 57779 can be increased through use of a heterologous signal sequence.

Fusion proteins can include all or a part of a serum protein, e.g., an IgG constant region, or human serum albumin.

The 23566, 33489, or 57779 fusion proteins of the invention can be incorporated into pharmaceutical compositions and administered to a subject in vivo. The 23566, 33489, or 57779 fusion proteins can be used to affect the bioavailability of a 23566, 33489, or 57779 substrate. 23566, 33489, or 57779 fusion proteins may be useful therapeutically for the treatment of disorders caused by, for example, (i) aberrant modification or mutation of a gene encoding a 23566, 33489, or 57779 protein; (ii) mis-regulation of the 23566, 33489, or 57779 gene; and (iii) aberrant post-translational modification of a 23566, 33489, or 57779 protein.

Moreover, the 23566, 33489, or 57779-fusion proteins of the invention can be used as immunogens to produce anti-23566, 33489, or 57779 antibodies in a subject, to purify 23566, 33489, or 57779 ligands and in screening assays to identify molecules which inhibit the interaction of 23566, 33489, or 57779 with a 23566, 33489, or 57779 substrate.

Expression vectors are commercially available that already encode a fusion moiety (e.g., a GST polypeptide). A 23566, 33489, or 57779-encoding nucleic acid can be cloned into such an expression vector such that the fusion moiety is linked in-frame to the 23566, 33489, or 57779 protein.

Variants of 23566, 33489, or 57779 Proteins

In another aspect, the invention also features a variant of a 23566, 33489, or 57779 polypeptide, e.g., which functions as an agonist (mimetics) or as an antagonist. Variants of the 23566, 33489, or 57779 proteins can be generated by mutagenesis, e.g., discrete point mutation, the insertion or deletion of sequences or the truncation of a 23566, 33489, or 57779 protein. An agonist of the 23566, 33489, or 57779 proteins can retain substantially the same, or a subset, of the biological activities of the naturally occurring form of a 23566, 33489, or 57779 protein. An antagonist of a 23566, 33489, or 57779 protein can inhibit one or more of the activities of the naturally occurring form of the 23566, 33489, or 57779 protein by, for example, competitively modulating a 23566, 33489, or 57779-mediated activity of a 23566, 33489, or 57779 protein. Thus, specific biological effects can be elicited by treatment with a variant of limited function. Preferably, treatment of a subject with a variant having a subset of the biological activities of the naturally occurring form of the protein has fewer side effects in a subject relative to treatment with the naturally occurring form of the 23566, 33489, or 57779 protein.

Variants of a 23566, 33489, or 57779 protein can be identified by screening combinatorial libraries of mutants, e.g., truncation mutants, of a 23566, 33489, or 57779 protein for agonist or antagonist activity.

Libraries of fragments e.g., N terminal, C terminal, or internal fragments, of a 23566, 33489, or 57779 protein coding sequence can be used to generate a variegated population of fragments for screening and subsequent selection of variants of a 23566, 33489, or 57779 protein. Variants in which a cysteine residues is added or deleted or in which a residue which is glycosylated is added or deleted are particularly preferred.

Methods for screening gene products of combinatorial libraries made by point mutations or truncation, and for screening cDNA libraries for gene products having a selected property are known in the art. Such methods are adaptable for rapid screening of the gene libraries generated by combinatorial mutagenesis of 23566, 33489, or 57779 proteins. Recursive ensemble mutagenesis (REM), a new technique which enhances the frequency of functional mutants in the libraries, can be used in combination with the screening assays to identify 23566, 33489, or 57779 variants (Arkin and Yourvan (1992) Proc. Natl. Acad. Sci. USA 89:7811-7815; Delgrave et al. (1993) Protein Engineering 6:327-331).

Cell based assays can be exploited to analyze a variegated 23566, 33489, or 57779 library. For example, a library of expression vectors can be transfected into a cell line, e.g., a cell line, which ordinarily responds to 23566, 33489, or 57779 in a substrate-dependent manner. The transfected cells are then contacted with 23566, 33489, or 57779 and the effect of the expression of the mutant on signaling by the 23566, 33489, or 57779 substrate can be detected. For 23566, the effect of the substrate could be detected, for example, by monitoring the response of the cells to a neuropeptide, wherein the effect of the neuropeptide in the absence of functional 23566 is to stimulate changes in cell shape and/or proliferation. For 33489, the effect of the substrate (e.g., Ca2+) could be to activate endothelial cell such that they could bind to blood cells, e.g., platelets, neutraphil, macrophages. For 57779, the effect of the substrate (e.g., Ca²⁺) could be to activate cell-cell adhesion. Plasmid DNA can then be recovered from the cells which score for inhibition, or alternatively, potentiation of signaling by the 23566, 33489, or 57779 substrate, and the individual clones further characterized.

In another aspect, the invention features a method of making a 23566, 33489, or 57779 polypeptide, e.g., a peptide having a non-wild type activity, e.g., an antagonist, agonist, or super agonist of a naturally occurring 23566, 33489, or 57779 polypeptide, e.g., a naturally occurring 23566, 33489, or 57779 polypeptide. The method includes: altering the sequence of a 23566, 33489, or 57779 polypeptide, e.g., altering the sequence, e.g., by substitution or deletion of one or more residues of a non-conserved region, a domain or residue disclosed herein, and testing the altered polypeptide for the desired activity.

In another aspect, the invention features a method of making a fragment or analog of a 23566, 33489, or 57779 polypeptide a biological activity of a naturally occurring 23566, 33489, or 57779 polypeptide. The method includes: altering the sequence, e.g., by substitution or deletion of one or more residues, of a 23566, 33489, or 57779 polypeptide, e.g., altering the sequence of a non-conserved region, or a domain or residue described herein, and testing the altered polypeptide for the desired activity.

Anti-23566, 33489 or 57779 Antibodies

In another aspect, the invention provides an anti-23566, 33489, or 57779 antibody, or a fragment thereof (e.g., an antigen-binding fragment thereof). The term “antibody” as used herein refers to an immunoglobulin molecule or immunologically active portion thereof, i.e., an antigen-binding portion. As used herein, the term “antibody” refers to a protein comprising at least one, and preferably two, heavy (H) chain variable regions (abbreviated herein as VH), and at least one and preferably two light (L) chain variable regions (abbreviated herein as VL). The VH and VL regions can be further subdivided into regions of hypervariability, termed “complementarity determining regions” (“CDR”), interspersed with regions that are more conserved, termed “framework regions” (FR). The extent of the framework region and CDR's has been precisely defined (see, Kabat, E. A., et al. (1991) Sequences of Proteins of Immunological Interest, Fifth Edition, U.S. Department of Health and Human Services, NIH Publication No. 91-3242, and Chothia, C. et al. (1987) J. Mol. Biol. 196:901-917, which are incorporated herein by reference). Each VH and VL is composed of three CDR's and four FRs, arranged from amino-terminus to carboxy-terminus in the following order: FR1, CDR1, FR2, CDR2, FR3, CDR3, FR4.

The anti-23566, 33489, or 57779 antibody can further include a heavy and light chain constant region, to thereby form a heavy and light immunoglobulin chain, respectively. In one embodiment, the antibody is a tetramer of two heavy immunoglobulin chains and two light immunoglobulin chains, wherein the heavy and light immunoglobulin chains are inter-connected by, e.g., disulfide bonds. The heavy chain constant region is comprised of three domains, CH1, CH2 and CH3. The light chain constant region is comprised of one domain, CL. The variable region of the heavy and light chains contains a binding domain that interacts with an antigen. The constant regions of the antibodies typically mediate the binding of the antibody to host tissues or factors, including various cells of the immune system (e.g., effector cells) and the first component (C1q) of the classical complement system.

As used herein, the term “immunoglobulin” refers to a protein consisting of one or more polypeptides substantially encoded by immunoglobulin genes. The recognized human immunoglobulin genes include the kappa, lambda, alpha (IgA1 and IgA2), gamma (IgG1, IgG2, IgG3, IgG4), delta, epsilon and mu constant region genes, as well as the myriad immunoglobulin variable region genes. Full-length immunoglobulin “light chains” (about 25 KDa or 214 amino acids) are encoded by a variable region gene at the NH2-terminus (about 110 amino acids) and a kappa or lambda constant region gene at the COOH—terminus. Full-length immunoglobulin “heavy chains” (about 50 KDa or 446 amino acids), are similarly encoded by a variable region gene (about 116 amino acids) and one of the other aforementioned constant region genes, e.g., gamma (encoding about 330 amino acids).

The term “antigen-binding fragment” of an antibody (or simply “antibody portion,” or “fragment”), as used herein, refers to one or more fragments of a full-length antibody that retain the ability to specifically bind to the antigen, e.g., 23566, 33489, or 57779 polypeptide or fragment thereof. Examples of antigen-binding fragments of the anti-23566, 33489, or 57779 antibody include, but are not limited to: (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also encompassed within the term “antigen-binding fragment” of an antibody. These antibody fragments are obtained using conventional techniques known to those with skill in the art, and the fragments are screened for utility in the same manner as are intact antibodies.

The anti-23566, 33489, or 57779 antibody can be a polyclonal or a monoclonal antibody. In other embodiments, the antibody can be recombinantly produced, e.g., produced by phage display or by combinatorial methods.

Phage display and combinatorial methods for generating anti-23566, 33489, or 57779 antibodies are known in the art (as described in, e.g., Ladner et al. U.S. Pat. No. 5,223,409; Kang et al. International Publication No. WO 92/18619; Dower et al. International Publication No. WO 91/17271; Winter et al. International Publication WO 92/20791; Markland et al. International Publication No. WO 92/15679; Breitling et al. International Publication WO 93/01288; McCafferty et al. International Publication No. WO 92/01047; Garrard et al.

International Publication No. WO 92/09690; Ladner et al. International Publication No. WO 90/02809; Fuchs et al. (1991) Bio/Technology 9:1370-1372; Hay et al. (1992) Hum Antibod Hybridomas 3:81-85; Huse et al. (1989) Science 246:1275-1281; Griffths et al. (1993) EMBO J. 12:725-734; Hawkins et al. (1992) J Mol Biol 226:889-896; Clackson et al. (1991) Nature 352:624-628; Gram et al. (1992) PNAS 89:3576-3580; Garrad et al. (1991) Bio/Technology 9:1373-1377; Hoogenboom et al. (1991) Nuc Acid Res 19:4133-4137; and Barbas et al. (1991) PNAS 88:7978-7982, the contents of all of which are incorporated by reference herein).

In one embodiment, the anti-23566, 33489, or 57779 antibody is a fully human antibody (e.g., an antibody made in a mouse which has been genetically engineered to produce an antibody from a human immunoglobulin sequence), or a non-human antibody, e.g., a rodent (mouse or rat), goat, primate (e.g., monkey), camel antibody. Preferably, the non-human antibody is a rodent (mouse or rat antibody). Method of producing rodent antibodies are known in the art.

Human monoclonal antibodies can be generated using transgenic mice carrying the human immunoglobulin genes rather than the mouse system. Splenocytes from these transgenic mice immunized with the antigen of interest are used to produce hybridomas that secrete human mAbs with specific affinities for epitopes from a human protein (see, e.g., Wood et al. International Application WO 91/00906, Kucherlapati et al. PCT publication WO 91/10741; Lonberg et al. International Application WO 92/03918; Kay et al. International Application 92/03917; Lonberg, N. et al. 1994 Nature 368:856-859; Green, L. L. et al. 1994 Nature Genet. 7:13-21; Morrison, S. L. et al. 1994 Proc. Natl. Acad. Sci. USA 81:6851-6855; Bruggeman et al. 1993 Year Immunol 7:33-40; Tuaillon et al. 1993 PNAS 90:3720-3724; Bruggeman et al. 1991 Eur J Immunol 21:1323-1326).

An anti-23566, 33489, or 57779 antibody can be one in which the variable region, or a portion thereof, e.g., the CDR's, are generated in a non-human organism, e.g., a rat or mouse. Chimeric, CDR-grafted, and humanized antibodies are within the invention. Antibodies generated in a non-human organism, e.g., a rat or mouse, and then modified, e.g., in the variable framework or constant region, to decrease antigenicity in a human are within the invention.

Chimeric antibodies can be produced by recombinant DNA techniques known in the art. For example, a gene encoding the Fc constant region of a murine (or other species) monoclonal antibody molecule is digested with restriction enzymes to remove the region encoding the murine Fc, and the equivalent portion of a gene encoding a human Fe constant region is substituted (see Robinson et al., International Patent Publication PCT/US86/02269; Akira, et al., European Patent Application 184,187; Taniguchi, M., European Patent Application 171,496; Morrison et al., European Patent Application 173,494; Neuberger et al., International Application WO 86/01533; Cabilly et al. U.S. Pat. No. 4,816,567; Cabilly et al., European Patent Application 125,023; Better et al. (1988 Science 240:1041-1043); Liu et al. (1987) PNAS 84:3439-3443; Liu et al., 1987, J. Immunol. 139:3521-3526; Sun et al. (1987) PNAS 84:214-218; Nishimura et al., 1987, Canc. Res. 47:999-1005; Wood et al. (1985) Nature 314:446-449; and Shaw et al., 1988, J. Natl Cancer Inst. 80:1553-1559).

A humanized or CDR-grafted antibody will have at least one or two but generally all three recipient CDR's (of heavy and or light immuoglobulin chains) replaced with a donor CDR. The antibody may be replaced with at least a portion of a non-human CDR or only some of the CDR's may be replaced with non-human CDR's. It is only necessary to replace the number of CDR's required for binding of the humanized antibody to a 23566, 33489, or 57779 or a fragment thereof. Preferably, the donor will be a rodent antibody, e.g., a rat or mouse antibody, and the recipient will be a human framework or a human consensus framework. Typically, the immunoglobulin providing the CDR's is called the “donor” and the immunoglobulin providing the framework is called the “acceptor.” In one embodiment, the donor immunoglobulin is a non-human (e.g., rodent). The acceptor framework is a naturally-occurring (e.g., a human) framework or a consensus framework, or a sequence about 85% or higher, preferably 90%, 95%, 99% or higher identical thereto. As used herein, the term “consensus sequence” refers to the sequence formed from the most frequently occurring amino acids (or nucleotides) in a family of related sequences (See e.g., Winnaker, From Genes to Clones (Verlagsgesellschaft, Weinheim, Germany 1987). In a family of proteins, each position in the consensus sequence is occupied by the amino acid occurring most frequently at that position in the family. If two amino acids occur equally frequently, either can be included in the consensus sequence. A “consensus framework” refers to the framework region in the consensus immunoglobulin sequence.

An antibody can be humanized by methods known in the art. Humanized antibodies can be generated by replacing sequences of the Fv variable region which are not directly involved in antigen binding with equivalent sequences from human Fv variable regions. General methods for generating humanized antibodies are provided by Morrison, S. L., 1985, Science 229:1202-1207, by Oi et al., 1986, BioTechniques 4:214, and by Queen et al. U.S. Pat. No. 5,585,089, U.S. Pat. No. 5,693,761 and U.S. Pat. No. 5,693,762, the contents of all of which are hereby incorporated by reference. Those methods include isolating, manipulating, and expressing the nucleic acid sequences that encode all or part of immunoglobulin Fv variable regions from at least one of a heavy or light chain. Sources of such nucleic acid are well known to those skilled in the art and, for example, may be obtained from a hybridoma producing an antibody against a 23566, 33489, or 57779 polypeptide or fragment thereof. The recombinant DNA encoding the humanized antibody, or fragment thereof, can then be cloned into an appropriate expression vector.

Humanized or CDR-grafted antibodies can be produced by CDR-grafting or CDR substitution, wherein one, two, or all CDR's of an immunoglobulin chain can be replaced. See e.g., U.S. Pat. No. 5,225,539; Jones et al. 1986 Nature 321:552-525; Verhoeyan et al. 1988 Science 239:1534; Beidler et al. 1988 J. Immunol. 141:4053-4060; Winter U.S. Pat. No. 5,225,539, the contents of all of which are hereby expressly incorporated by reference. Winter describes a CDR-grafting method that may be used to prepare the humanized antibodies of the present invention (UK Patent Application GB 2188638A, filed on Mar. 26, 1987; Winter U.S. Pat. No. 5,225,539), the contents of which is expressly incorporated by reference.

Also within the scope of the invention are humanized antibodies in which specific amino acids have been substituted, deleted or added. Preferred humanized antibodies have amino acid substitutions in the framework region, such as to improve binding to the antigen. For example, a humanized antibody will have framework residues identical to the donor framework residue or to another amino acid other than the recipient framework residue. To generate such antibodies, a selected, small number of acceptor framework residues of the humanized immunoglobulin chain can be replaced by the corresponding donor amino acids. Preferred locations of the substitutions include amino acid residues adjacent to the CDR, or which are capable of interacting with a CDR (see e.g., U.S. Pat. No. 5,585,089). Criteria for selecting amino acids from the donor are described in U.S. Pat. No. 5,585,089, e.g., columns 12-16 of U.S. Pat. No. 5,585,089, the e.g., columns 12-16 of U.S. Pat. No. 5,585,089, the contents of which are hereby incorporated by reference. Other techniques for humanizing antibodies are described in Padlan et al. EP 519596 A1, published on Dec. 23, 1992.

In preferred embodiments an antibody can be made by immunizing with purified 23566, 33489, or 57779 antigen, or a fragment thereof, e.g., a fragment described herein, membrane associated antigen, tissue, e.g., crude tissue preparations, whole cells, preferably living cells, lysed cells, or cell fractions, e.g., membrane fractions.

A full-length 23566, 33489, or 57779 protein or, antigenic peptide fragment of 23566, 33489, or 57779 can be used as an immunogen or can be used to identify anti-23566, 33489, or 57779 antibodies made with other immunogens, e.g., cells, membrane preparations, and the like. The antigenic peptide of 23566, 33489, or 57779 should include at least 8 amino acid residues of the amino acid sequence shown in SEQ ID NO:74, SEQ ID NO:77, or SEQ ID NO:80 and encompasses an epitope of 23566, 33489, or 57779. Preferably, the antigenic peptide includes at least 10 amino acid residues, more preferably at least 15 amino acid residues, even more preferably at least 20 amino acid residues, and most preferably at least 30 amino acid residues.

Fragments of 23566 can be used, e.g., to characterize the specificity of an antibody or to make immunogens. For example, fragments of 23566 which include residues about 70 to 100, about 170 to 200, or about 285 to 310 can be used to make antibodies against hydrophilic regions of the 23566 protein. Similarly, fragments of 23566 which include residues 41 to 68, about 130 to 150, or about 270 to 285 can be used to make an antibody against a hydrophobic region of the 23566 protein; fragments of 23566 which include residues about 152 to 444 can be used to make an antibody against an extracellular region of the 23566 protein; fragments of 23566 which include residues about 1 to 133 can be used to make an antibody against an intracellular region of the 23566 protein; and fragment of 23566 which include residues about 180 to 443 or about 229 to 251 can be used to make an antibody against the zinc carboxypeptidase domain or the zinc carboxypeptidase zinc-binding region 1 signature domain, respectively, of the 23566 protein.

Fragments of 33489 can be used, e.g., to characterize the specificity of an antibody or to make immunogens. For example, fragments of 33489 which include residues from about 89 to 96, from about 218 to 232, or from about 245 to 253 of SEQ ID NO:77 can be used to make antibodies against hydrophilic regions of the 33489 protein. Similarly, fragments of 33489 which include residues from about 58 to 80, from about 204 to 212, or from about 266 to 282 of SEQ ID NO:77 can be used to make an antibody against a hydrophobic region of the 33489 protein; a fragment of 33489 which includes residues about 283 to 295 of SEQ ID NO:77 can be used to make an antibody against an extracellular region of the 33489 protein; a fragment of 33489 which includes residues about 1 to 265 of SEQ ID NO:77 can be used to make an antibody against an intracellular region of the 33489 protein; a fragment of 33489 which includes residues about 103 to 285 of SEQ ID NO:77 can be used to make an antibody against the scramblase region of the 33489 protein.

Fragments of 57779 can be used, e.g., as immunogens or used to characterize the specificity of an antibody. For example, fragments of 57779 which include residues from about 408 to 420, from about 434 to 455, and from about 719 to 740 of SEQ ID NO:80, antibodies against hydrophilic regions of the 57779 protein. Similarly, fragments of 57779 which include residues from about 4 to 16, from about 676 to 701, and from about 832 to 852 of SEQ ID NO:80 can be used to make an antibody against a hydrophobic region of the 57779 protein; fragments of 57779 which include residues about 26 to 676, and about 860 to 873 of SEQ ID NO:80 can be used to make an antibody against an extracellular region of the 57779 protein; a fragment of 57779 which includes residues about 702 to 834 of SEQ ID NO:80 can be used to make an antibody against an intracellular region of the 57779 protein; a fragment of 57779 which include residues about 25 to 120, 134 to 229, 243 to 337, 354 to 444, 458 to 554, and 573 to 663 of SEQ ID NO:80 can be used to make an antibody against a cadherin domain of the 57779 protein.

Antibodies reactive with, or specific for, any of these regions, or other regions or domains described herein are provided.

Antibodies that bind only native 23566, 33489, or 57779 protein, only denatured or otherwise non-native 23566, 33489, or 57779 protein, or which bind both, are with in the invention. Antibodies with linear or conformational epitopes are within the invention. Conformational epitopes can sometimes be identified by identifying antibodies that bind to native but not denatured 23566, 33489, or 57779 protein.

Preferred epitopes encompassed by the antigenic peptide are regions of 23566, 33489, or 57779 are located on the surface of the protein, e.g., hydrophilic regions, as well as regions with high antigenicity. For example, an Emini surface probability analysis of the human 23566, 33489, or 57779 protein sequence can be used to indicate the regions that have a particularly high probability of being localized to the surface of the 23566, 33489, or 57779 protein and are thus likely to constitute surface residues useful for targeting antibody production.

In a preferred embodiment the antibody can bind to the extracellular portion of the 23566, 33489, or 57779 protein, e.g., it can bind to a whole cell which expresses the 23566, 33489, or 57779 protein. In another embodiment, the antibody binds an intracellular portion of the 23566, 33489, or 57779 protein. In preferred embodiments antibodies can bind one or more of purified antigen, membrane associated antigen, tissue, e.g., tissue sections, whole cells, preferably living cells, lysed cells, cell fractions, e.g., membrane fractions.

The anti-23566, 33489, or 57779 antibody can be a single chain antibody. A single-chain antibody (scFV) may be engineered (see, for example, Colcher, D. et al. (1999) Ann NY Acad Sci 880:263-80; and Reiter, Y. (1996) Clin Cancer Res 2:245-52). The single chain antibody can be dimerized or multimerized to generate multivalent antibodies having specificities for different epitopes of the same target 23566, 33489, or 57779 protein.

In a preferred embodiment the antibody has effector function and/or can fix complement. In other embodiments the antibody does not recruit effector cells; or fix complement.

In a preferred embodiment, the antibody has reduced or no ability to bind an Fc receptor. For example, it is a isotype or subtype, fragment or other mutant, which does not support binding to an Fc receptor, e.g., it has a mutagenized or deleted Fe receptor binding region.

In a preferred embodiment, an anti-23566 antibody alters (e.g., increases or decreases) the carboxypeptidase activity of a 23566 polypeptide. For example, the antibody can bind at or in proximity to the active site, e.g., to an epitope that includes a residue located from about 180 to 443 or about 229 to 251 of SEQ ID NO:74.

In a preferred embodiment, an anti-33489 antibody alters (e.g., increases or decreases) the scramblase activity of a 33489 polypeptide. For example, the antibody can bind at or in proximity to the active site or a site distict from the active site that moves, e.g., changes position relative to other parts of the molecule, when the scramblase domain is transferring a lipid from one memebrane leaflet to another.

In a preferred embodiment, an anti-57779 antibody alters (e.g., increases or decreases) the cell-cell adhesion activity mediated by a 57779 polypeptide. For example, the antibody can bind at or in proximity to: one of the calcium binding epitopes; one of the cadherin domains, e.g., the first or second cadherin domain, e.g., about amino acid residues 25 to 120 or 134 to 229 of SEQ ID NO:80; or to the RGD cell attachment motif, e.g., about amino acid residues 181 to 183 of SEQ ID NO:80.

The antibody can be coupled to a toxin, e.g., a polypeptide toxin, e,g, ricin or diphtheria toxin or active fragment hereof, or a radioactive nucleus, or imaging agent, e.g. a radioactive, enzymatic, or other, e.g., imaging agent, e.g., a NMR contrast agent. Labels which produce detectable radioactive emissions or fluorescence are preferred.

An anti-23566, 33489, or 57779 antibody (e.g., monoclonal antibody) can be used to isolate 23566, 33489, or 57779 by standard techniques, such as affinity chromatography or immunoprecipitation. Moreover, an anti-23566, 33489, or 57779 antibody can be used to detect 23566, 33489, or 57779 protein (e.g., in a cellular lysate or cell supernatant) in order to evaluate the abundance and pattern of expression of the protein. Anti-23566, 33489, or 57779 antibodies can be used diagnostically to monitor protein levels in tissue as part of a clinical testing procedure, e.g., to determine the efficacy of a given treatment regimen. Detection can be facilitated by coupling (i.e., physically linking) the antibody to a detectable substance (i.e., antibody labelling). Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

The invention also includes a nucleic acid which encodes an anti-23566, 33489, or 57779 antibody, e.g., an anti-23566, 33489, or 57779 antibody described herein. Also included are vectors which include the nucleic acid and cells transformed with the nucleic acid, particularly cells which are useful for producing an antibody, e.g., mammalian cells, e.g. CHO or lymphatic cells.

The invention also includes cell lines, e.g., hybridomas, which make an anti-23566, 33489, or 57779 antibody, e.g., an antibody described herein, and method of using said cells to make a 23566, 33489, or 57779 antibody.

23566, 33489, and 57779 Recombinant Expression Vectors, Host Cells and Genetically Engineered Cells

In another aspect, the invention includes, vectors, preferably expression vectors, containing a nucleic acid encoding a polypeptide described herein. As used herein, the term “vector” refers to a nucleic acid molecule capable of transporting another nucleic acid to which it has been linked and can include a plasmid, cosmid or viral vector. The vector can be capable of autonomous replication or it can integrate into a host DNA. Viral vectors include, e.g., replication defective retroviruses, adenoviruses and adeno-associated viruses.

A vector can include a 23566, 33489, or 57779 nucleic acid in a form suitable for expression of the nucleic acid in a host cell. Preferably the recombinant expression vector includes one or more regulatory sequences operatively linked to the nucleic acid sequence to be expressed. The term “regulatory sequence” includes promoters, enhancers and other expression control elements (e.g., polyadenylation signals). Regulatory sequences include those which direct constitutive expression of a nucleotide sequence, as well as tissue-specific regulatory and/or inducible sequences. The design of the expression vector can depend on such factors as the choice of the host cell to be transformed, the level of expression of protein desired, and the like. The expression vectors of the invention can be introduced into host cells to thereby produce proteins or polypeptides, including fusion proteins or polypeptides, encoded by nucleic acids as described herein (e.g., 23566, 33489, or 57779 proteins, mutant forms of 23566, 33489, or 57779 proteins, fusion proteins, and the like).

The recombinant expression vectors of the invention can be designed for expression of 23566, 33489, or 57779 proteins in prokaryotic or eukaryotic cells. For example, polypeptides of the invention can be expressed in E. coli, insect cells (e.g., using baculovirus expression vectors), yeast cells or mammalian cells. Suitable host cells are discussed further in Goeddel, (1990) Gene Expression Technology: Methods in Enzymology 185, Academic Press, San Diego, Calif. Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith, D. B. and Johnson, K. S. (1988) Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

Purified fusion proteins can be used in 23566, 33489, or 57779 activity assays, (e.g., direct assays or competitive assays described in detail below), or to generate antibodies specific for 23566, 33489, or 57779 proteins. In a preferred embodiment, a fusion protein expressed in a retroviral expression vector of the present invention can be used to infect bone marrow cells which are subsequently transplanted into irradiated recipients. The pathology of the subject recipient is then examined after sufficient time has passed (e.g., six weeks).

To maximize recombinant protein expression in E. coli is to express the protein in a host bacteria with an impaired capacity to proteolytically cleave the recombinant protein (Gottesman, S., (1990) Gene Expression Technology: Methods in Enzymology 185, Academic Press, San Diego, Calif. 119-128). Another strategy is to alter the nucleic acid sequence of the nucleic acid to be inserted into an expression vector so that the individual codons for each amino acid are those preferentially utilized in E. coli (Wada et al., (1992) Nucleic Acids Res. 20:2111-2118). Such alteration of nucleic acid sequences of the invention can be carried out by standard DNA synthesis techniques.

The 23566, 33489, or 57779 expression vector can be a yeast expression vector, a vector for expression in insect cells, e.g., a baculovirus expression vector or a vector suitable for expression in mammalian cells.

When used in mammalian cells, the expression vector's control functions can be provided by viral regulatory elements. For example, commonly used promoters are derived from polyoma, Adenovirus 2, cytomegalovirus and Simian Virus 40.

In another embodiment, the promoter is an inducible promoter, e.g., a promoter regulated by a steroid hormone, by a polypeptide hormone (e.g., by means of a signal transduction pathway), or by a heterologous polypeptide (e.g., the tetracycline-inducible systems, “Tet-On” and “Tet-Off”; see, e.g., Clontech Inc., CA, Gossen and Bujard (1992) Proc. Natl. Acad. Sci. USA 89:5547, and Paillard (1989) Human Gene Therapy 9:983).

In another embodiment, the recombinant mammalian expression vector is capable of directing expression of the nucleic acid preferentially in a particular cell type (e.g., tissue-specific regulatory elements are used to express the nucleic acid). Non-limiting examples of suitable tissue-specific promoters include the albumin promoter (liver-specific; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) Proc. Natl. Acad. Sci. USA 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, for example, the murine hox promoters (Kessel and Gruss (1990) Science 249:374-379) and the α-fetoprotein promoter (Campes and Tilghman (1989) Genes Dev. 3:537-546).

The invention further provides a recombinant expression vector comprising a DNA molecule of the invention cloned into the expression vector in an antisense orientation. Regulatory sequences (e.g., viral promoters and/or enhancers) operatively linked to a nucleic acid cloned in the antisense orientation can be chosen which direct the constitutive, tissue specific or cell type specific expression of antisense RNA in a variety of cell types. The antisense expression vector can be in the form of a recombinant plasmid, phagemid or attenuated virus.

Another aspect the invention provides a host cell which includes a nucleic acid molecule described herein, e.g., a 23566, 33489, or 57779 nucleic acid molecule within a recombinant expression vector or a 23566, 33489, or 57779 nucleic acid molecule containing sequences which allow it to homologously recombine into a specific site of the host cell's genome. The terms “host cell” and “recombinant host cell” are used interchangeably herein. Such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, a 23566, 33489, or 57779 protein can be expressed in bacterial cells (such as E. coli), insect cells, yeast or mammalian cells (such as Chinese hamster ovary cells (CHO) or COS cells (African green monkey kidney cells CV-1 origin SV40 cells; Gluzman (1981) Cell 23:175-182)). Other suitable host cells are known to those skilled in the art.

Vector DNA can be introduced into host cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation.

A host cell of the invention can be used to produce (i.e., express) a 23566, 33489, or 57779 protein. Accordingly, the invention further provides methods for producing a 23566, 33489, or 57779 protein using the host cells of the invention. In one embodiment, the method includes culturing the host cell of the invention (into which a recombinant expression vector encoding a 23566, 33489, or 57779 protein has been introduced) in a suitable medium such that a 23566, 33489, or 57779 protein is produced. In another embodiment, the method further includes isolating a 23566, 33489, or 57779 protein from the medium or the host cell.

In another aspect, the invention features, a cell or purified preparation of cells which include a 23566, 33489, or 57779 transgene, or which otherwise misexpress 23566, 33489, or 57779. The cell preparation can consist of human or non-human cells, e.g., rodent cells, e.g., mouse or rat cells, rabbit cells, or pig cells. In preferred embodiments, the cell or cells include a 23566, 33489, or 57779 transgene, e.g., a heterologous form of a 23566, 33489, or 57779, e.g., a gene derived from humans (in the case of a non-human cell). The 23566, 33489, or 57779 transgene can be misexpressed, e.g., overexpressed or underexpressed. In other preferred embodiments, the cell or cells include a gene that mis-expresses an endogenous 23566, 33489, or 57779, e.g., a gene the expression of which is disrupted, e.g., a knockout. Such cells can serve as a model for studying disorders that are related to mutated or mis-expressed 23566, 33489, or 57779 alleles or for use in drug screening.

In another aspect, the invention features, a human cell, e.g., a hematopoietic stem cell, a neuronal cell, or an endothelial cell, transformed with nucleic acid which encodes a 23566, 33489, or 57779 polypeptide.

Also provided are cells, preferably human cells, e.g., human hematopoietic, neuronal, endothelial, or fibroblast cells, in which an endogenous 23566, 33489, or 57779 is under the control of a regulatory sequence that does not normally control the expression of the endogenous 23566, 33489, or 57779 gene. The expression characteristics of an endogenous gene within a cell, e.g., a cell line or microorganism, can be modified by inserting a heterologous DNA regulatory element into the genome of the cell such that the inserted regulatory element is operably linked to the endogenous 23566, 33489, or 57779 gene. For example, an endogenous 23566, 33489, or 57779 gene which is “transcriptionally silent,” e.g., not normally expressed, or expressed only at very low levels, may be activated by inserting a regulatory element which is capable of promoting the expression of a normally expressed gene product in that cell. Techniques such as targeted homologous recombinations, can be used to insert the heterologous DNA as described in, e.g., Chappel, U.S. Pat. No. 5,272,071; WO 91/06667, published in May 16, 1991.

In a preferred embodiment, recombinant cells described herein can be used for replacement therapy in a subject. For example, a nucleic acid encoding a 23566, 33489, or 57779 polypeptide operably linked to an inducible promoter (e.g., a steroid hormone receptor-regulated promoter) is introduced into a human or nonhuman, e.g., mammalian, e.g., porcine recombinant cell. The cell is cultivated and encapsulated in a biocompatible material, such as poly-lysine alginate, and subsequently implanted into the subject. See, e.g., Lanza (1996) Nat. Biotechnol. 14:1107; Joki et al. (2001) Nat. Biotechnol. 19:35; and U.S. Pat. No. 5,876,742. Production of 23566, 33489, or 57779 polypeptide can be regulated in the subject by administering an agent (e.g., a steroid hormone) to the subject. In another preferred embodiment, the implanted recombinant cells express and secrete an antibody specific for a 23566, 33489, or 57779 polypeptide. The antibody can be any antibody or any antibody derivative described herein.

23566, 33489, and 57779 Transgenic Animals

The invention provides non-human transgenic animals. Such animals are useful for studying the function and/or activity of a 23566, 33489, or 57779 protein and for identifying and/or evaluating modulators of 23566, 33489, or 57779 activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, and the like. A transgene is exogenous DNA or a rearrangement, e.g., a deletion of endogenous chromosomal DNA, which preferably is integrated into or occurs in the genome of the cells of a transgenic animal. A transgene can direct the expression of an encoded gene product in one or more cell types or tissues of the transgenic animal, other transgenes, e.g., a knockout, reduce expression. Thus, a transgenic animal can be one in which an endogenous 23566, 33489, or 57779 gene has been altered by, e.g., by homologous recombination between the endogenous gene and an exogenous DNA molecule introduced into a cell of the animal, e.g., an embryonic cell of the animal, prior to development of the animal.

Intronic sequences and polyadenylation signals can also be included in the transgene to increase the efficiency of expression of the transgene. A tissue-specific regulatory sequence(s) can be operably linked to a transgene of the invention to direct expression of a 23566, 33489, or 57779 protein to particular cells. A transgenic founder animal can be identified based upon the presence of a 23566, 33489, or 57779 transgene in its genome and/or expression of 23566, 33489, or 57779 mRNA in tissues or cells of the animals. A transgenic founder animal can then be used to breed additional animals carrying the transgene. Moreover, transgenic animals carrying a transgene encoding a 23566, 33489, or 57779 protein can further be bred to other transgenic animals carrying other transgenes.

23566, 33489, or 57779 proteins or polypeptides can be expressed in transgenic animals or plants, e.g., a nucleic acid encoding the protein or polypeptide can be introduced into the genome of an animal. In preferred embodiments the nucleic acid is placed under the control of a tissue specific promoter, e.g., a milk or egg specific promoter, and recovered from the milk or eggs produced by the animal. Suitable animals are mice, pigs, cows, goats, and sheep.

The invention also includes a population of cells from a transgenic animal, as discussed, e.g., below.

Uses of 23566, 33489, and 57779

The nucleic acid molecules, proteins, protein homologues, and antibodies described herein can be used in one or more of the following methods: a) screening assays; b) predictive medicine (e.g., diagnostic assays, prognostic assays, monitoring clinical trials, and pharmacogenetics); and c) methods of treatment (e.g., therapeutic and prophylactic).

The isolated nucleic acid molecules of the invention can be used, for example, to express a 23566, 33489, or 57779 protein (e.g., via a recombinant expression vector in a host cell in gene therapy applications), to detect a 23566, 33489, or 57779 mRNA (e.g., in a biological sample) or a genetic alteration in a 23566, 33489, or 57779 gene, and to modulate 23566, 33489, or 57779 activity, as described further below. The 23566, 33489, or 57779 proteins can be used to treat disorders characterized by insufficient or excessive production of a 23566, 33489, or 57779 substrate or production of 23566, 33489, or 57779 inhibitors. In addition, the 23566, 33489, or 57779 proteins can be used to screen for naturally occurring 23566, 33489, or 57779 substrates, to screen for drugs or compounds which modulate 23566, 33489, or 57779 activity, as well as to treat disorders characterized by insufficient or excessive production of 23566, 33489, or 57779 protein or production of 23566, 33489, or 57779 protein forms which have decreased, aberrant or unwanted activity compared to 23566, 33489, or 57779 wild type protein (e.g., inflammatory disorders, neurological disorders, cardiovascular disorders (e.g., disorders of the heart and/or blood vessels), blood clotting disorders, or cellular proliferation and/or differentiation disorders). Moreover, the anti-23566, 33489, or 57779 antibodies of the invention can be used to detect and isolate 23566, 33489, or 57779 proteins, regulate the bioavailability of 23566, 33489, or 57779 proteins, and modulate 23566, 33489, or 57779 activity.

Isolated nucleic 23566 polypeptides can be used in vitro, for example, to produce such products as digested polypeptides/proteins, or to process prohormones. Isolated 57779 polypeptides can be used, for example to generate antibodies which identify cells associated with the brain cortex or hypothalamus

A method of evaluating a compound for the ability to interact with, e.g., bind, a subject 23566, 33489, or 57779 polypeptide is provided. The method includes: contacting the compound with the subject 23566, 33489, or 57779 polypeptide; and evaluating ability of the compound to interact with, e.g., to bind or form a complex with the subject 23566, 33489, or 57779 polypeptide. This method can be performed in vitro, e.g., in a cell free system, or in vivo, e.g., in a two-hybrid interaction trap assay. This method can be used to identify naturally occurring molecules that interact with subject 23566, 33489, or 57779 polypeptide. It can also be used to find natural or synthetic inhibitors of subject 23566, 33489, or 57779 polypeptide. Screening methods are discussed in more detail below.

23566, 33489, and 57779 Screening Assays

The invention provides methods (also referred to herein as “screening assays”) for identifying modulators, i.e., candidate or test compounds or agents (e.g., proteins, peptides, peptidomimetics, peptoids, small molecules or other drugs) which bind to 23566, 33489, or 57779 proteins, have a stimulatory or inhibitory effect on, for example, 23566, 33489, or 57779 expression or 23566, 33489, or 57779 activity, or have a stimulatory or inhibitory effect on, for example, the expression or activity of a 23566, 33489, or 57779 substrate. Compounds thus identified can be used to modulate the activity of target gene products (e.g., 23566, 33489, or 57779 genes) in a therapeutic protocol, to elaborate the biological function of the target gene product, or to identify compounds that disrupt normal target gene interactions.

In one embodiment, the invention provides assays for screening candidate or test compounds which are substrates of a 23566, 33489, or 57779 protein or polypeptide or a biologically active portion thereof. In another embodiment, the invention provides assays for screening candidate or test compounds that bind to or modulate an activity of a 23566, 33489, or 57779 protein or polypeptide or a biologically active portion thereof.

In one embodiment, an activity of a 23566 protein can be assayed by measuring the rate of hydrolysis cause by the enzyme against a range of carboxypeptidase substrates, e.g., measuring hydrolysis in vitro spectrophotometrically in 50 mM Tris-HCl, 0.5 M NaCl, pH 7.5, at 25° C. (see, for example, Joshi et al., J Biol Chem 274: 9803-9811 (1999), and Gingras et al., J Biol Chem 274: 11742-11750 (1999)).

In one embodiment, an activity of a 33489 protein can be assayed by detecting scramblase activity as described in Basse et al. (1996) J Biol Chem 271:17205-17210. An exemplary assay is described briefly as follows.

Liposomes are prepared by drying phosphatidylcholine and phosphatidylserine in a 9:1 molar ratio under a stream of nitrogen. The dried mixture is rehydrated in a standard buffer, e.g., 100 mM Tris.HCl pH 7.4, 100 mM KCl, 0.1 mM EGTA. The outer leaflet of the liposomes can be labeled by the addition of NBD-phosphoserine (1-oleoyl-2-[6(7-nitrobenz-2-oxa-1,3-diazol-4-yl)amino]caproyl-sn-glycero-3-phosphoserine) or NBD-phosphocholine (1-oleoyl-2-[6(7-nitrobenz-2-oxa-1,3-diazol-4-yl)amino]caproyl-sn-glycero-3-phosphocholine) from a DMSO stock solution. A protein sample to be assayed is prepared in a buffer with 60 mM octylglucoside detergent. The sample is combined with the liposome preparation and dialyzed to remove the octylglucoside. If sample activation is also assayed, an activator, e.g., Ca²⁺ or other signal can be applied, e.g., into a stirred fluorescence cuvette. Fluorescence is monitored with time, by excitation at 470 nm, and monitoring emission at 532 nm. After 90 to 120 seconds, dithionite (Sigma Co., MO) is added to quench fluorophores on the outer leaflet. Scramblase activity will result in the transfer of labeled molecules to the inner leaflet, and thus increased fluorescence relative to a control lacking active protein. The assay can also be performed by labeling the inner leaflet, and determining if increased quenching is observed.

In one embodiment, an activity of a 57779 protein can be assayed using methods well known in the art, e.g., the method described in Sano et al. (1993) EMBO J. 12:2249.

The test compounds of the present invention can be obtained using any of the numerous approaches in combinatorial library methods known in the art, including: biological libraries; peptoid libraries (libraries of molecules having the functionalities of peptides, but with a novel, non-peptide backbone which are resistant to enzymatic degradation but which nevertheless remain bioactive; see, e.g., Zuckermann, R. N. et al. (1994) J. Med. Chem. 37:2678-85); spatially addressable parallel solid phase or solution phase libraries; synthetic library methods requiring deconvolution; the ‘one-bead one-compound’ library method; and synthetic library methods using affinity chromatography selection. The biological library and peptoid library approaches are limited to peptide libraries, while the other four approaches are applicable to peptide, non-peptide oligomer or small molecule libraries of compounds (Lam (1997) Anticancer Drug Des. 12:145).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and Gallop et al. (1994) J. Med. Chem. 37:1233.

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner, U.S. Pat. No. 5,223,409), spores (Ladner U.S. Pat. No. 5,223,409), plasmids (Cull et al. (1992) Proc Natl Acad Sci USA 89:1865-1869) or on phage (Scott and Smith (1990) Science 249:386-390; Devlin (1990) Science 249:404-406; Cwirla et al. (1990) Proc. Natl. Acad. Sci. 87:6378-6382; Felici (1991) J. Mol. Biol. 222:301-310; Ladner supra.).

In one embodiment, an assay is a cell-based assay in which a cell which expresses a 23566, 33489, or 57779 protein or biologically active portion thereof is contacted with a test compound, and the ability of the test compound to modulate 23566, 33489, or 57779 activity is determined. Determining the ability of the test compound to modulate 23566, 33489, or 57779 activity can be accomplished by monitoring, for example, peptide hydrolysis activity (e.g., carboxy terminal peptide hydrolysis activity), scramblase activity, or cellular adhesive activity (e.g., homotypic cellular adhesion). The cell, for example, can be of mammalian origin, e.g., human.

The ability of the test compound to modulate 23566, 33489, or 57779 binding to a compound, e.g., a 23566, 33489, or 57779 substrate, or to bind to 23566, 33489, or 57779 can also be evaluated. This can be accomplished, for example, by coupling the compound, e.g., the substrate, with a radioisotope or enzymatic label such that binding of the compound, e.g., the substrate, to 23566, 33489, or 57779 can be determined by detecting the labeled compound, e.g., substrate, in a complex. Alternatively, 23566, 33489, or 57779 could be coupled with a radioisotope or enzymatic label to monitor the ability of a test compound to modulate 23566, 33489, or 57779 binding to a 23566, 33489, or 57779 substrate in a complex. For example, compounds (e.g., 23566, 33489, or 57779 substrates) can be labeled with ¹²⁵I, ³⁵S, ¹⁴C, or ³H, either directly or indirectly, and the radioisotope detected by direct counting of radioemmission or by scintillation counting. Alternatively, compounds can be enzymatically labeled with, for example, horseradish peroxidase, alkaline phosphatase, or luciferase, and the enzymatic label detected by determination of conversion of an appropriate substrate to product.

The ability of a compound (e.g., a 23566, 33489, or 57779 substrate) to interact with 23566, 33489, or 57779 with or without the labeling of any of the interactants can be evaluated. For example, a microphysiometer can be used to detect the interaction of a compound with 23566, 33489, or 57779 without the labeling of either the compound or the 23566, 33489, or 57779. McConnell, H. M. et al. (1992) Science 257:1906-1912. As used herein, a “microphysiometer” (e.g., Cytosensor) is an analytical instrument that measures the rate at which a cell acidifies its environment using a light-addressable potentiometric sensor (LAPS). Changes in this acidification rate can be used as an indicator of the interaction between a compound and 23566, 33489, or 57779.

In yet another embodiment, a cell-free assay is provided in which a 23566, 33489, or 57779 protein or biologically active portion thereof is contacted with a test compound and the ability of the test compound to bind to the 23566, 33489, or 57779 protein or biologically active portion thereof is evaluated. Preferred biologically active portions of the 23566, 33489, or 57779 proteins to be used in assays of the present invention include fragments which participate in interactions with non-23566, 33489, or 57779 molecules, e.g., fragments with high surface probability scores.

Soluble and/or membrane-bound forms of isolated proteins (e.g., 23566, 33489, or 57779 proteins or biologically active portions thereof) can be used in the cell-free assays of the invention. When membrane-bound forms of the protein are used, it may be desirable to utilize a solubilizing agent. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)ₙ, 3-[(3-cholamidopropyl)dimethylamminio]-1-propane sulfonate (CHAPS), 3-[(3-cholamidopropyl)dimethylamminio]-2-hydroxy-1-propane sulfonate (CHAPSO), or N-dodecyl=N,N-dimethyl-3-ammonio-1-propane sulfonate.

Cell-free assays involve preparing a reaction mixture of the target gene protein and the test compound under conditions and for a time sufficient to allow the two components to interact and bind, thus forming a complex that can be removed and/or detected.

The interaction between two molecules can also be detected, e.g., using fluorescence energy transfer (FET) (see, for example, Lakowicz et al., U.S. Pat. No. 5,631,169; Stavrianopoulos, et al., U.S. Pat. No. 4,868,103). A fluorophore label on the first, ‘donor’ molecule is selected such that its emitted fluorescent energy will be absorbed by a fluorescent label on a second, ‘acceptor’ molecule, which in turn is able to fluoresce due to the absorbed energy. Alternately, the ‘donor’ protein molecule may simply utilize the natural fluorescent energy of tryptophan residues. Labels are chosen that emit different wavelengths of light, such that the ‘acceptor’ molecule label may be differentiated from that of the ‘donor’. Since the efficiency of energy transfer between the labels is related to the distance separating the molecules, the spatial relationship between the molecules can be assessed. In a situation in which binding occurs between the molecules, the fluorescent emission of the ‘acceptor’ molecule label in the assay should be maximal. An FET binding event can be conveniently measured through standard fluorometric detection means well known in the art (e.g., using a fluorimeter).

In another embodiment, determining the ability of the 23566, 33489, or 57779 protein to bind to a target molecule can be accomplished using real-time Biomolecular Interaction Analysis (BIA) (see, e.g., Sjolander, S. and Urbaniczky, C. (1991) Anal. Chem. 63:2338-2345 and Szabo et al. (1995) Curr. Opin. Struct. Biol. 5:699-705). “Surface plasmon resonance” or “BIA” detects biospecific interactions in real time, without labeling any of the interactants (e.g., BIAcore). Changes in the mass at the binding surface (indicative of a binding event) result in alterations of the refractive index of light near the surface (the optical phenomenon of surface plasmon resonance (SPR)), resulting in a detectable signal which can be used as an indication of real-time reactions between biological molecules.

In one embodiment, the target gene product or the test substance is anchored onto a solid phase. The target gene product/test compound complexes anchored on the solid phase can be detected at the end of the reaction. Preferably, the target gene product can be anchored onto a solid surface, and the test compound, (which is not anchored), can be labeled, either directly or indirectly, with detectable labels discussed herein.

It may be desirable to immobilize either 23566, 33489, or 57779, an anti-23566, 33489, or 57779 antibody or its target molecule to facilitate separation of complexed from uncomplexed forms of one or both of the proteins, as well as to accommodate automation of the assay. Binding of a test compound to a 23566, 33489, or 57779 protein, or interaction of a 23566, 33489, or 57779 protein with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided which adds a domain that allows one or both of the proteins to be bound to a matrix. For example, glutathione-S-transferase/23566, 33489, or 57779 fusion proteins or glutathione-S-transferase/target fusion proteins can be adsorbed onto glutathione sepharose beads (Sigrna Chemical, St. Louis, Mo.) or glutathione derivatized microtiter plates, which are then combined with the test compound or the test compound and either the non-adsorbed target protein or 23566, 33489, or 57779 protein, and the mixture incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described above. Alternatively, the complexes can be dissociated from the matrix, and the level of 23566, 33489, or 57779 binding or activity determined using standard techniques.

Other techniques for immobilizing either a 23566, 33489, or 57779 protein or a target molecule on matrices include using conjugation of biotin and streptavidin. Biotinylated 23566, 33489, or 57779 protein or target molecules can be prepared from biotin-NHS(N-hydroxy-succinimide) using techniques known in the art (e.g., biotinylation kit, Pierce Chemicals, Rockford, Ill.), and immobilized in the wells of streptavidin-coated 96 well plates (Pierce Chemical).

In order to conduct the assay, the non-immobilized component is added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways. Where the previously non-immobilized component is pre-labeled, the detection of label immobilized on the surface indicates that complexes were formed. Where the previously non-immobilized component is not pre-labeled, an indirect label can be used to detect complexes anchored on the surface; e.g., using a labeled antibody specific for the immobilized component (the antibody, in turn, can be directly labeled or indirectly labeled with, e.g., a labeled anti-Ig antibody).

In one embodiment, this assay is performed utilizing antibodies reactive with 23566, 33489, or 57779 protein or target molecules but which do not interfere with binding of the 23566, 33489, or 57779 protein to its target molecule. Such antibodies can be derivatized to the wells of the plate, and unbound target or 23566, 33489, or 57779 protein trapped in the wells by antibody conjugation. Methods for detecting such complexes, in addition to those described above for the GST-immobilized complexes, include immunodetection of complexes using antibodies reactive with the 23566, 33489, or 57779 protein or target molecule, as well as enzyme-linked assays which rely on detecting an enzymatic activity associated with the 23566, 33489, or 57779 protein or target molecule.

Alternatively, cell free assays can be conducted in a liquid phase. In such an assay, the reaction products are separated from unreacted components, by any of a number of standard techniques, including but not limited to: differential centrifugation (see, for example, Rivas, G., and Minton, A. P., (1993) Trends Biochem Sci 18:284-7); chromatography (gel filtration chromatography, ion-exchange chromatography); electrophoresis (see, e.g., Ausubel, F. et al., eds. Current Protocols in Molecular Biology 1999, J. Wiley: New York.); and immunoprecipitation (see, for example, Ausubel, F. et al., eds. (1999) Current Protocols in Molecular Biology, J. Wiley: New York). Such resins and chromatographic techniques are known to one skilled in the art (see, e.g., Heegaard, N. H., (1998) J Mol Recognit 11: 141-8; Hage, D. S., and Tweed, S. A. (1997) J Chromatogr B Biomed Sci Appl. 699:499-525). Further, fluorescence energy transfer may also be conveniently utilized, as described herein, to detect binding without further purification of the complex from solution.

In a preferred embodiment, the assay includes contacting the 23566, 33489, or 57779 protein or biologically active portion thereof with a known compound which binds 23566, 33489, or 57779 to form an assay mixture, contacting the assay mixture with a test compound, and determining the ability of the test compound to interact with a 23566, 33489, or 57779 protein, wherein determining the ability of the test compound to interact with a 23566, 33489, or 57779 protein includes determining the ability of the test compound to preferentially bind to 23566, 33489, or 57779 or biologically active portion thereof, or to modulate the activity of a target molecule, as compared to the known compound.

The target gene products of the invention can, in vivo, interact with one or more cellular or extracellular macromolecules, such as proteins. For the purposes of this discussion, such cellular and extracellular macromolecules are referred to herein as “binding partners.”

Compounds that disrupt such interactions can be useful in regulating the activity of the target gene product. Such compounds can include, but are not limited to molecules such as antibodies, peptides, and small molecules. The preferred target genes/products for use in this embodiment are the 23566, 33489, or 57779 genes herein identified. In an alternative embodiment, the invention provides methods for determining the ability of the test compound to modulate the activity of a 23566, 33489, or 57779 protein through modulation of the activity of a downstream effector of a 23566, 33489, or 57779 target molecule. For example, the activity of the effector molecule on an appropriate target can be determined, or the binding of the effector to an appropriate target can be determined, as previously described.

To identify compounds that interfere with the interaction between the target gene product and its cellular or extracellular binding partner(s), a reaction mixture containing the target gene product and the binding partner is prepared, under conditions and for a time sufficient, to allow the two products to form complex. In order to test an inhibitory agent, the reaction mixture is provided in the presence and absence of the test compound. The test compound can be initially included in the reaction mixture, or can be added at a time subsequent to the addition of the target gene and its cellular or extracellular binding partner. Control reaction mixtures are incubated without the test compound or with a placebo. The formation of any complexes between the target gene product and the cellular or extracellular binding partner is then detected. The formation of a complex in the control reaction, but not in the reaction mixture containing the test compound, indicates that the compound interferes with the interaction of the target gene product and the interactive binding partner. Additionally, complex formation within reaction mixtures containing the test compound and normal target gene product can also be compared to complex formation within reaction mixtures containing the test compound and mutant target gene product. This comparison can be important in those cases wherein it is desirable to identify compounds that disrupt interactions of mutant but not normal target gene products.

These assays can be conducted in a heterogeneous or homogeneous format. Heterogeneous assays involve anchoring either the target gene product or the binding partner onto a solid phase, and detecting complexes anchored on the solid phase at the end of the reaction. In homogeneous assays, the entire reaction is carried out in a liquid phase. In either approach, the order of addition of reactants can be varied to obtain different information about the compounds being tested. For example, test compounds that interfere with the interaction between the target gene products and the binding partners, e.g., by competition, can be identified by conducting the reaction in the presence of the test substance. Alternatively, test compounds that disrupt preformed complexes, e.g., compounds with higher binding constants that displace one of the components from the complex, can be tested by adding the test compound to the reaction mixture after complexes have been formed. The various formats are briefly described below.

In a heterogeneous assay system, either the target gene product or the interactive cellular or extracellular binding partner, is anchored onto a solid surface (e.g., a microtiter plate), while the non-anchored species is labeled, either directly or indirectly. The anchored species can be immobilized by non-covalent or covalent attachments. Alternatively, an immobilized antibody specific for the species to be anchored can be used to anchor the species to the solid surface.

In order to conduct the assay, the partner of the immobilized species is exposed to the coated surface with or without the test compound. After the reaction is complete, unreacted components are removed (e.g., by washing) and any complexes formed will remain immobilized on the solid surface. Where the non-immobilized species is pre-labeled, the detection of label immobilized on the surface indicates that complexes were formed. Where the non-immobilized species is not pre-labeled, an indirect label can be used to detect complexes anchored on the surface; e.g., using a labeled antibody specific for the initially non-immobilized species (the antibody, in turn, can be directly labeled or indirectly labeled with, e.g., a labeled anti-Ig antibody). Depending upon the order of addition of reaction components, test compounds that inhibit complex formation or that disrupt preformed complexes can be detected.

Alternatively, the reaction can be conducted in a liquid phase in the presence or absence of the test compound, the reaction products separated from unreacted components, and complexes detected; e.g., using an immobilized antibody specific for one of the binding components to anchor any complexes formed in solution, and a labeled antibody specific for the other partner to detect anchored complexes. Again, depending upon the order of addition of reactants to the liquid phase, test compounds that inhibit complex or that disrupt preformed complexes can be identified.

In an alternate embodiment of the invention, a homogeneous assay can be used. For example, a preformed complex of the target gene product and the interactive cellular or extracellular binding partner product is prepared in that either the target gene products or their binding partners are labeled, but the signal generated by the label is quenched due to complex formation (see, e.g., U.S. Pat. No. 4,109,496 that utilizes this approach for immunoassays). The addition of a test substance that competes with and displaces one of the species from the preformed complex will result in the generation of a signal above background. In this way, test substances that disrupt target gene product-binding partner interaction can be identified.

In yet another aspect, the 23566, 33489, or 57779 proteins can be used as “bait proteins” in a two-hybrid assay or three-hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Biotechniques 14:920-924; Iwabuchi et al. (1993) Oncogene 8:1693-1696; and Brent WO94/10300), to identify other proteins, which bind to or interact with 23566, 33489, or 57779 (“23566, 33489, or 57779-binding proteins” or “23566, 33489, or 57779-bp”) and are involved in 23566, 33489, or 57779 activity. Such 23566, 33489, or 57779-bps can be activators or inhibitors of signals by the 23566, 33489, or 57779 proteins or 23566, 33489, or 57779 targets as, for example, downstream elements of a 23566, 33489, or 57779-mediated signaling pathway.

The two-hybrid system is based on the modular nature of most transcription factors, which consist of separable DNA-binding and activation domains. Briefly, the assay utilizes two different DNA constructs. In one construct, the gene that codes for a 23566, 33489, or 57779 protein is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. (Alternatively the: 23566, 33489, or 57779 protein can be the fused to the activator domain.) If the “bait” and the “prey” proteins are able to interact, in vivo, forming a 23566, 33489, or 57779-dependent complex, the DNA-binding and activation domains of the transcription factor are brought into close proximity. This proximity allows transcription of a reporter gene (e.g., lacZ) which is operably linked to a transcriptional regulatory site responsive to the transcription factor. Expression of the reporter gene can be detected and cell colonies containing the functional transcription factor can be isolated and used to obtain the cloned gene which encodes the protein which interacts with the 23566, 33489, or 57779 protein.

In another embodiment, modulators of 23566, 33489, or 57779 expression are identified. For example, a cell or cell free mixture is contacted with a candidate compound and the expression of 23566, 33489, or 57779 mRNA or protein evaluated relative to the level of expression of 23566, 33489, or 57779 mRNA or protein in the absence of the candidate compound. When expression of 23566, 33489, or 57779 mRNA or protein is greater in the presence of the candidate compound than in its absence, the candidate compound is identified as a stimulator of 23566, 33489, or 57779 mRNA or protein expression. Alternatively, when expression of 23566, 33489, or 57779 mRNA or protein is less (statistically significantly less) in the presence of the candidate compound than in its absence, the candidate compound is identified as an inhibitor of 23566, 33489, or 57779 mRNA or protein expression. The level of 23566, 33489, or 57779 mRNA or protein expression can be determined by methods described herein for detecting 23566, 33489, or 57779 mRNA or protein.

In another aspect, the invention pertains to a combination of two or more of the assays described herein. For example, a modulating agent can be identified using a cell-based or a cell free assay, and the ability of the agent to modulate the activity of a 23566, 33489, or 57779 protein can be confirmed in vivo, e.g., in an animal such as an animal model for inflammatory disorders, neurological disorders, cardiovascular disorders (e.g., disorders of the heart and/or blood vessels), blood clotting disorders, or cellular proliferation and/or differentiation disorders.

This invention further pertains to novel agents identified by the above-described screening assays. Accordingly, it is within the scope of this invention to further use an agent identified as described herein (e.g., a 23566, 33489, or 57779 modulating agent, an antisense 23566, 33489, or 57779 nucleic acid molecule, a 23566, 33489, or 57779-specific antibody, or a 23566, 33489, or 57779-binding partner) in an appropriate animal model to determine the efficacy, toxicity, side effects, or mechanism of action, of treatment with such an agent. Furthermore, novel agents identified by the above-described screening assays can be used for treatments as described herein.

23566, 33489, and 57779 Detection Assays

Portions or fragments of the nucleic acid sequences identified herein can be used as polynucleotide reagents. For example, these sequences can be used to: (i) map their respective genes on a chromosome e.g., to locate gene regions associated with genetic disease or to associate 23566, 33489, or 57779 with a disease; (ii) identify an individual from a minute biological sample (tissue typing); and (iii) aid in forensic identification of a biological sample. These applications are described in the subsections below.

23566, 33489, and 57779 Chromosome Mapping

The 23566, 33489, or 57779 nucleotide sequences or portions thereof can be used to map the location of the 23566, 33489, or 57779 genes on a chromosome. This process is called chromosome mapping. Chromosome mapping is useful in correlating the 23566, 33489, or 57779 sequences with genes associated with disease.

Briefly, 23566, 33489, or 57779 genes can be mapped to chromosomes by preparing PCR primers (preferably 15-25 bp in length) from the 23566, 33489, or 57779 nucleotide sequences. These primers can then be used for PCR screening of somatic cell hybrids containing individual human chromosomes. Only those hybrids containing the human gene corresponding to the 23566, 33489, or 57779 sequences will yield an amplified fragment.

A panel of somatic cell hybrids in which each cell line contains either a single human chromosome or a small number of human chromosomes, and a full set of mouse chromosomes, can allow easy mapping of individual genes to specific human chromosomes. (D'Eustachio P. et al. (1983) Science 220:919-924).

Other mapping strategies e.g., in situ hybridization (described in Fan, Y. et al. (1990) Proc. Natl. Acad. Sci. USA, 87:6223-27), pre-screening with labeled flow-sorted chromosomes, and pre-selection by hybridization to chromosome specific cDNA libraries can be used to map 23566, 33489, or 57779 to a chromosomal location.

Fluorescence in situ hybridization (FISH) of a DNA sequence to a metaphase chromosomal spread can further be used to provide a precise chromosomal location in one step. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases will suffice to get good results at a reasonable amount of time. For a review of this technique, see Verma et al., Human Chromosomes: A Manual of Basic Techniques ((1988) Pergamon Press, New York).

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. (Such data are found, for example, in V. McKusick, Mendelian Inheritance in Man, available on-line through Johns Hopkins University Welch Medical Library). The relationship between a gene and a disease, mapped to the same chromosomal region, can then be identified through linkage analysis (co-inheritance of physically adjacent genes), described in, for example, Egeland, J. et al. (1987) Nature, 325:783-787.

Moreover, differences in the DNA sequences between individuals affected and unaffected with a disease associated with the 23566, 33489, or 57779 gene, can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

23566, 33489, and 57779 Tissue Typing

23566, 33489, or 57779 sequences can be used to identify individuals from biological samples using, e.g., restriction fragment length polymorphism (RFLP). In this technique, an individual's genomic DNA is digested with one or more restriction enzymes, the fragments separated, e.g., in a Southern blot, and probed to yield bands for identification. The sequences of the present invention are useful as additional DNA markers for RFLP (described in U.S. Pat. No. 5,272,057).

Furthermore, the sequences of the present invention can also be used to determine the actual base-by-base DNA sequence of selected portions of an individual's genome. Thus, the 23566, 33489, or 57779 nucleotide sequences described herein can be used to prepare two PCR primers from the 5′ and 3′ ends of the sequences. These primers can then be used to amplify an individual's DNA and subsequently sequence it. Panels of corresponding DNA sequences from individuals, prepared in this manner, can provide unique individual identifications, as each individual will have a unique set of such DNA sequences due to allelic differences.

Allelic variation occurs to some degree in the coding regions of these sequences, and to a greater degree in the noncoding regions. Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences of SEQ ID NO:73, SEQ ID NO:76, or SEQ ID NO:79 can provide positive individual identification with a panel of perhaps 10 to 1,000 primers which each yield a noncoding amplified sequence of 100 bases. If predicted coding sequences, such as those in SEQ ID NO:75, SEQ ID NO:78, or SEQ ID NO:81 are used, a more appropriate number of primers for positive individual identification would be 500-2,000.

If a panel of reagents from 23566, 33489, or 57779 nucleotide sequences described herein is used to generate a unique identification database for an individual, those same reagents can later be used to identify tissue from that individual. Using the unique identification database, positive identification of the individual, living or dead, can be made from extremely small tissue samples.

Use of Partial 23566, 33489, or 57779 Sequences in Forensic Biology

DNA-based identification techniques can also be used in forensic biology. To make such an identification, PCR technology can be used to amplify DNA sequences taken from very small biological samples such as tissues, e.g., hair or skin, or body fluids, e.g., blood, saliva, or semen found at a crime scene. The amplified sequence can then be compared to a standard, thereby allowing identification of the origin of the biological sample.

The sequences of the present invention can be used to provide polynucleotide reagents, e.g., PCR primers, targeted to specific loci in the human genome, which can enhance the reliability of DNA-based forensic identifications by, for example, providing another “identification marker” (i.e. another DNA sequence that is unique to a particular individual). As mentioned above, actual base sequence information can be used for identification as an accurate alternative to patterns formed by restriction enzyme generated fragments. Sequences targeted to noncoding regions of SEQ ID NO:73, SEQ ID NO:76, or SEQ ID NO:79 (e.g., fragments derived from the noncoding regions of SEQ ID NO:73, SEQ ID NO:76, or SEQ ID NO:79 having a length of at least 20 bases, preferably at least 30 bases) are particularly appropriate for this use.

The 23566, 33489, or 57779 nucleotide sequences described herein can further be used to provide polynucleotide reagents, e.g., labeled or labelable probes which can be used in, for example, an in situ hybridization technique, to identify a specific tissue. This can be very useful in cases where a forensic pathologist is presented with a tissue of unknown origin. Panels of such 23566, 33489, or 57779 probes can be used to identify tissue by species and/or by organ type.

In a similar fashion, these reagents, e.g., 23566, 33489, or 57779 primers or probes can be used to screen tissue culture for contamination (i.e. screen for the presence of a mixture of different types of cells in a culture).

Predictive Medicine of 23566, 33489, and 57779

The present invention also pertains to the field of predictive medicine in which diagnostic assays, prognostic assays, and monitoring clinical trials are used for prognostic (predictive) purposes to thereby treat an individual.

Generally, the invention provides, a method of determining if a subject is at risk for a disorder related to a lesion in or the misexpression of a gene which encodes 23566, 33489, or 57779.

Such disorders include, e.g., a disorder associated with the misexpression of a 23566, 33489, or 57779 gene; inflammatory disorders, neurological disorders, cardiovascular disorders (e.g., disorders of the heart and/or blood vessels), blood clotting disorders, or cellular proliferation and/or differentiation disorders. The method includes one or more of the following:

detecting, in a tissue of the subject, the presence or absence of a mutation which affects the expression of the 23566, 33489, or 57779 gene, or detecting the presence or absence of a mutation in a region which controls the expression of the gene, e.g., a mutation in the 5′ control region;

detecting, in a tissue of the subject, the presence or absence of a mutation which alters the structure of the 23566, 33489, or 57779 gene;

detecting, in a tissue of the subject, the misexpression of the 23566, 33489, or 57779 gene, at the mRNA level, e.g., detecting a non-wild type level of a mRNA;

detecting, in a tissue of the subject, the misexpression of the gene, at the protein level, e.g., detecting a non-wild type level of a 23566, 33489, or 57779 polypeptide.

In preferred embodiments the method includes: ascertaining the existence of at least one of: a deletion of one or more nucleotides from the 23566, 33489, or 57779 gene; an insertion of one or more nucleotides into the gene, a point mutation, e.g., a substitution of one or more nucleotides of the gene, a gross chromosomal rearrangement of the gene, e.g., a translocation, inversion, or deletion.

For example, detecting the genetic lesion can include: (i) providing a probe/primer including an oligonucleotide containing a region of nucleotide sequence which hybridizes to a sense or antisense sequence from SEQ ID NO:73, SEQ ID NO:76, or SEQ ID NO:79, or naturally occurring mutants thereof or 5′ or 3′ flanking sequences naturally associated with the 23566, 33489, or 57779 gene; (ii) exposing the probe/primer to nucleic acid of the tissue; and detecting, by hybridization, e.g., in situ hybridization, of the probe/primer to the nucleic acid, the presence or absence of the genetic lesion.

In preferred embodiments detecting the misexpression includes ascertaining the existence of at least one of: an alteration in the level of a messenger RNA transcript of the 23566, 33489, or 57779 gene; the presence of a non-wild type splicing pattern of a messenger RNA transcript of the gene; or a non-wild type level of 23566, 33489, or 57779.

Methods of the invention can be used prenatally or to determine if a subject's offspring will be at risk for a disorder.

In preferred embodiments the method includes determining the structure of a 23566, 33489, or 57779 gene, an abnormal structure being indicative of risk for the disorder.

In preferred embodiments the method includes contacting a sample from the subject with an antibody to the 23566, 33489, or 57779 protein or a nucleic acid, which hybridizes specifically with the gene. These and other embodiments are discussed below.

Diagnostic and Prognostic Assays of 23566, 33489, and 57779

Diagnostic and prognostic assays of the invention include method for assessing the expression level of 23566, 33489, or 57779 molecules and for identifying variations and mutations in the sequence of 23566, 33489, or 57779 molecules.

Expression Monitoring and Profiling:

The presence, level, or absence of 23566, 33489, or 57779 protein or nucleic acid in a biological sample can be evaluated by obtaining a biological sample from a test subject and contacting the biological sample with a compound or an agent capable of detecting 23566, 33489, or 57779 protein or nucleic acid (e.g., mRNA, genomic DNA) that encodes 23566, 33489, or 57779 protein such that the presence of 23566, 33489, or 57779 protein or nucleic acid is detected in the biological sample. The term “biological sample” includes tissues, cells and biological fluids isolated from a subject, as well as tissues, cells and fluids present within a subject. A preferred biological sample is serum. The level of expression of the 23566, 33489, or 57779 gene can be measured in a number of ways, including, but not limited to: measuring the mRNA encoded by the 23566, 33489, or 57779 genes; measuring the amount of protein encoded by the 23566, 33489, or 57779 genes; or measuring the activity of the protein encoded by the 23566, 33489, or 57779 genes.

The level of mRNA corresponding to the 23566, 33489, or 57779 gene in a cell can be determined both by in situ and by in vitro formats.

The isolated mRNA can be used in hybridization or amplification assays that include, but are not limited to, Southern or Northern analyses, polymerase chain reaction analyses and probe arrays. One preferred diagnostic method for the detection of mRNA levels involves contacting the isolated mRNA with a nucleic acid molecule (probe) that can hybridize to the mRNA encoded by the gene being detected. The nucleic acid probe can be, for example, a full-length 23566, 33489, or 57779 nucleic acid, such as the nucleic acid of SEQ ID NO:73, SEQ ID NO:76, or SEQ ID NO:79, or a portion thereof, such as an oligonucleotide of at least 7, 15, 30, 50, 100, 250 or 500 nucleotides in length and sufficient to specifically hybridize under stringent conditions to 23566, 33489, or 57779 mRNA or genomic DNA. The probe can be disposed on an address of an array, e.g., an array described below. Other suitable probes for use in the diagnostic assays are described herein.

In one format, mRNA (or cDNA) is immobilized on a surface and contacted with the probes, for example by running the isolated mRNA on an agarose gel and transferring the mRNA from the gel to a membrane, such as nitrocellulose. In an alternative format, the probes are immobilized on a surface and the mRNA (or cDNA) is contacted with the probes, for example, in a two-dimensional gene chip array described below. A skilled artisan can adapt known mRNA detection methods for use in detecting the level of mRNA encoded by the 23566, 33489, or 57779 genes.

The level of mRNA in a sample that is encoded by one of 23566, 33489, or 57779 can be evaluated with nucleic acid amplification, e.g., by rtPCR (Mullis (1987) U.S. Pat. No. 4,683,202), ligase chain reaction (Barany (1991) Proc. Natl. Acad. Sci. USA 88:189-193), self sustained sequence replication (Guatelli et al., (1990) Proc. Natl. Acad. Sci. USA 87:1874-1878), transcriptional amplification system (Kwoh et al., (1989), Proc. Natl. Acad. Sci. USA 86:1173-1177), Q-Beta Replicase (Lizardi et al., (1988) Bio/Technology 6:1197), rolling circle replication (Lizardi et al., U.S. Pat. No. 5,854,033) or any other nucleic acid amplification method, followed by the detection of the amplified molecules using techniques known in the art. As used herein, amplification primers are defined as being a pair of nucleic acid molecules that can anneal to 5′ or 3′ regions of a gene (plus and minus strands, respectively, or vice-versa) and contain a short region in between. In general, amplification primers are from about 10 to 30 nucleotides in length and flank a region from about 50 to 200 nucleotides in length. Under appropriate conditions and with appropriate reagents, such primers permit the amplification of a nucleic acid molecule comprising the nucleotide sequence flanked by the primers.

For in situ methods, a cell or tissue sample can be prepared/processed and immobilized on a support, typically a glass slide, and then contacted with a probe that can hybridize to mRNA that encodes the 23566, 33489, or 57779 gene being analyzed.

In another embodiment, the methods further contacting a control sample with a compound or agent capable of detecting 23566, 33489, or 57779 mRNA, or genomic DNA, and comparing the presence of 23566, 33489, or 57779 mRNA or genomic DNA in the control sample with the presence of 23566, 33489, or 57779 mRNA or genomic DNA in the test sample. In still another embodiment, serial analysis of gene expression, as described in U.S. Pat. No. 5,695,937, is used to detect 23566, 33489, or 57779 transcript levels.

A variety of methods can be used to determine the level of protein encoded by 23566, 33489, or 57779. In general, these methods include contacting an agent that selectively binds to the protein, such as an antibody with a sample, to evaluate the level of protein in the sample. In a preferred embodiment, the antibody bears a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with a detectable substance. Examples of detectable substances are provided herein.

The detection methods can be used to detect 23566, 33489, or 57779 protein in a biological sample in vitro as well as in vivo. In vitro techniques for detection of 23566, 33489, or 57779 protein include enzyme linked immunosorbent assays (ELISAs), immunoprecipitations, immunofluorescence, enzyme immunoassay (EIA), radioimmunoassay (RIA), and Western blot analysis. In vivo techniques for detection of 23566, 33489, or 57779 protein include introducing into a subject a labeled anti-23566, 33489, or 57779 antibody. For example, the antibody can be labeled with a radioactive marker whose presence and location in a subject can be detected by standard imaging techniques. In another embodiment, the sample is labeled, e.g., biotinylated and then contacted to the antibody, e.g., an anti-23566, 33489, or 57779 antibody positioned on an antibody array (as described below). The sample can be detected, e.g., with avidin coupled to a fluorescent label.

In another embodiment, the methods further include contacting the control sample with a compound or agent capable of detecting 23566, 33489, or 57779 protein, and comparing the presence of 23566, 33489, or 57779 protein in the control sample with the presence of 23566, 33489, or 57779 protein in the test sample.

The invention also includes kits for detecting the presence of 23566, 33489, or 57779 in a biological sample. For example, the kit can include a compound or agent capable of detecting 23566, 33489, or 57779 protein or mRNA in a biological sample; and a standard. The compound or agent can be packaged in a suitable container. The kit can further comprise instructions for using the kit to detect 23566, 33489, or 57779 protein or nucleic acid.

For antibody-based kits, the kit can include: (1) a first antibody (e.g., attached to a solid support) which binds to a polypeptide corresponding to a marker of the invention; and, optionally, (2) a second, different antibody which binds to either the polypeptide or the first antibody and is conjugated to a detectable agent.

For oligonucleotide-based kits, the kit can include: (1) an oligonucleotide, e.g., a detectably labeled oligonucleotide, which hybridizes to a nucleic acid sequence encoding a polypeptide corresponding to a marker of the invention or (2) a pair of primers useful for amplifying a nucleic acid molecule corresponding to a marker of the invention. The kit can also includes a buffering agent, a preservative, or a protein stabilizing agent. The kit can also includes components necessary for detecting the detectable agent (e.g., an enzyme or a substrate). The kit can also contain a control sample or a series of control samples which can be assayed and compared to the test sample contained. Each component of the kit can be enclosed within an individual container and all of the various containers can be within a single package, along with instructions for interpreting the results of the assays performed using the kit.

The diagnostic methods described herein can identify subjects having, or at risk of developing, a disease or disorder associated with misexpressed or aberrant or unwanted 23566, 33489, or 57779 expression or activity. As used herein, the term “unwanted” includes an unwanted phenomenon involved in a biological response such as an inflammatory disorder, neurological disorder, cardiovascular disorder (e.g., disorder of the heart and/or blood vessels), a blood clotting disorder, or deregulated cell proliferation.

In one embodiment, a disease or disorder associated with aberrant or unwanted 23566, 33489, or 57779 expression or activity is identified. A test sample is obtained from a subject and 23566, 33489, or 57779 protein or nucleic acid (e.g., mRNA or genomic DNA) is evaluated, wherein the level, e.g., the presence or absence, of 23566, 33489, or 57779 protein or nucleic acid is diagnostic for a subject having or at risk of developing a disease or disorder associated with aberrant or unwanted 23566, 33489, or 57779 expression or activity. As used herein, a “test sample” refers to a biological sample obtained from a subject of interest, including a biological fluid (e.g., serum), cell sample, or tissue.

The prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant or unwanted 23566, 33489, or 57779 expression or activity. For example, such methods can be used to determine whether a subject can be effectively treated with an agent for an inflammatory disorder, neurological disorder, cardiovascular disorder (e.g., disorder of the heart and/or blood vessels), blood clotting disorders disorder, or cellular proliferation and/or differentiation disorder.

In another aspect, the invention features a computer medium having a plurality of digitally encoded data records. Each data record includes a value representing the level of expression of 23566, 33489, or 57779 in a sample, and a descriptor of the sample. The descriptor of the sample can be an identifier of the sample, a subject from which the sample was derived (e.g., a patient), a diagnosis, or a treatment (e.g., a preferred treatment). In a preferred embodiment, the data record further includes values representing the level of expression of genes other than 23566, 33489, or 57779 (e.g., other genes associated with a 23566, 33489, or 57779-disorder, or other genes on an array). The data record can be structured as a table, e.g., a table that is part of a database such as a relational database (e.g., a SQL database of the Oracle or Sybase database environments).

Also featured is a method of evaluating a sample. The method includes providing a sample, e.g., from the subject, and determining a gene expression profile of the sample, wherein the profile includes a value representing the level of 23566, 33489, or 57779 expression. The method can further include comparing the value or the profile (i.e., multiple values) to a reference value or reference profile. The gene expression profile of the sample can be obtained by any of the methods described herein (e.g., by providing a nucleic acid from the sample and contacting the nucleic acid to an array). The method can be used to diagnose a disorder, e.g., an inflammatory disorder, neurological disorder, cardiovascular disorder (e.g., disorder of the heart and/or blood vessels), blood clotting disorder, or cellular proliferation and/or differentiation disorder, in a subject wherein an increase or decrease in 23566, 33489, or 57779 expression is an indication that the subject has or is disposed to having such a disorder. The method can be used to monitor a treatment for a disorder, e.g., an inflammatory disorder, neurological disorder, cardiovascular disorder (e.g., disorder of the heart and/or blood vessels), blood clotting disorder, or cellular proliferation and/or differentiation disorder, in a subject. For example, the gene expression profile can be determined for a sample from a subject undergoing treatment. The profile can be compared to a reference profile or to a profile obtained from the subject prior to treatment or prior to onset of the disorder (see, e.g., Golub et al. (1999) Science 286:531).

In yet another aspect, the invention features a method of evaluating a test compound (see also, “Screening Assays”, above). The method includes providing a cell and a test compound; contacting the test compound to the cell; obtaining a subject expression profile for the contacted cell; and comparing the subject expression profile to one or more reference profiles. The profiles include a value representing the level of 23566, 33489, or 57779 expression. In a preferred embodiment, the subject expression profile is compared to a target profile, e.g., a profile for a normal cell or for desired condition of a cell. The test compound is evaluated favorably if the subject expression profile is more similar to the target profile than an expression profile obtained from an uncontacted cell.

In another aspect, the invention features, a method of evaluating a subject. The method includes: a) obtaining a sample from a subject, e.g., from a caregiver, e.g., a caregiver who obtains the sample from the subject; b) determining a subject expression profile for the sample. Optionally, the method further includes either or both of steps: c) comparing the subject expression profile to one or more reference expression profiles; and d) selecting the reference profile most similar to the subject reference profile. The subject expression profile and the reference profiles include a value representing the level of 23566, 33489, or 57779 expression. A variety of routine statistical measures can be used to compare two reference profiles. One possible metric is the length of the distance vector that is the difference between the two profiles. Each of the subject and reference profile is represented as a multi-dimensional vector, wherein each dimension is a value in the profile.

The method can further include transmitting a result to a caregiver. The result can be the subject expression profile, a result of a comparison of the subject expression profile with another profile, a most similar reference profile, or a descriptor of any of the aforementioned. The result can be transmitted across a computer network, e.g., the result can be in the form of a computer transmission, e.g., a computer data signal embedded in a carrier wave.

Also featured is a computer medium having executable code for effecting the following steps: receive a subject expression profile; access a database of reference expression profiles; and either i) select a matching reference profile most similar to the subject expression profile or ii) determine at least one comparison score for the similarity of the subject expression profile to at least one reference profile. The subject expression profile, and the reference expression profiles each include a value representing the level of 23566, 33489, or 57779 expression.

23566, 33489, and 57779 Arrays and uses Thereof

In another aspect, the invention features an array that includes a substrate having a plurality of addresses. At least one address of the plurality includes a capture probe that binds specifically to a 23566, 33489, or 57779 molecule (e.g., a 23566, 33489, or 57779 nucleic acid or a 23566, 33489, or 57779 polypeptide). The array can have a density of at least than 10, 50, 100, 200, 500, 1,000, 2,000, or 10,000 or more addresses/cm², and ranges between. In a preferred embodiment, the plurality of addresses includes at least 10, 100, 500, 1,000, 5,000, 10,000, 50,000 addresses. In a preferred embodiment, the plurality of addresses includes equal to or less than 10, 100, 500, 1,000, 5,000, 10,000, or 50,000 addresses. The substrate can be a two-dimensional substrate such as a glass slide, a wafer (e.g., silica or plastic), a mass spectroscopy plate, or a three-dimensional substrate such as a gel pad. Addresses in addition to address of the plurality can be disposed on the array.

In a preferred embodiment, at least one address of the plurality includes a nucleic acid capture probe that hybridizes specifically to a 23566, 33489, or 57779 nucleic acid, e.g., the sense or anti-sense strand. In one preferred embodiment, a subset of addresses of the plurality of addresses has a nucleic acid capture probe for 23566, 33489, or 57779. Each address of the subset can include a capture probe that hybridizes to a different region of a 23566, 33489, or 57779 nucleic acid. In another preferred embodiment, addresses of the subset include a capture probe for a 23566, 33489, or 57779 nucleic acid. Each address of the subset is unique, overlapping, and complementary to a different variant of 23566, 33489, or 57779 (e.g., an allelic variant, or all possible hypothetical variants). The array can be used to sequence 23566, 33489, or 57779 by hybridization (see, e.g., U.S. Pat. No. 5,695,940).

An array can be generated by various methods, e.g., by photolithographic methods (see, e.g., U.S. Pat. Nos. 5,143,854; 5,510,270; and 5,527,681), mechanical methods (e.g., directed-flow methods as described in U.S. Pat. No. 5,384,261), pin-based methods (e.g., as described in U.S. Pat. No. 5,288,514), and bead-based techniques (e.g., as described in PCT US/93/04145).

In another preferred embodiment, at least one address of the plurality includes a polypeptide capture probe that binds specifically to a 23566, 33489, or 57779 polypeptide or fragment thereof. The polypeptide can be a naturally-occurring interaction partner of 23566, 33489, or 57779 polypeptide. Preferably, the polypeptide is an antibody, e.g., an antibody described herein (see “Anti-23566, 33489, or 57779 Antibodies,” above), such as a monoclonal antibody or a single-chain antibody.

In another aspect, the invention features a method of analyzing the expression of 23566, 33489, or 57779. The method includes providing an array as described above; contacting the array with a sample and detecting binding of a 23566, 33489, or 57779-molecule (e.g., nucleic acid or polypeptide) to the array. In a preferred embodiment, the array is a nucleic acid array. Optionally the method further includes amplifying nucleic acid from the sample prior or during contact with the array.

In another embodiment, the array can be used to assay gene expression in a tissue to ascertain tissue specificity of genes in the array, particularly the expression of 23566, 33489, or 57779. If a sufficient number of diverse samples is analyzed, clustering (e.g., hierarchical clustering, k-means clustering, Bayesian clustering and the like) can be used to identify other genes which are co-regulated with 23566, 33489, or 57779. For example, the array can be used for the quantitation of the expression of multiple genes. Thus, not only tissue specificity, but also the level of expression of a battery of genes in the tissue is ascertained. Quantitative data can be used to group (e.g., cluster) genes on the basis of their tissue expression per se and level of expression in that tissue.

For example, array analysis of gene expression can be used to assess the effect of cell-cell interactions on 23566, 33489, or 57779 expression. A first tissue can be perturbed and nucleic acid from a second tissue that interacts with the first tissue can be analyzed. In this context, the effect of one cell type on another cell type in response to a biological stimulus can be determined, e.g., to monitor the effect of cell-cell interaction at the level of gene expression.

In another embodiment, cells are contacted with a therapeutic agent. The expression profile of the cells is determined using the array, and the expression profile is compared to the profile of like cells not contacted with the agent. For example, the assay can be used to determine or analyze the molecular basis of an undesirable effect of the therapeutic agent. If an agent is administered therapeutically to treat one cell type but has an undesirable effect on another cell type, the invention provides an assay to determine the molecular basis of the undesirable effect and thus provides the opportunity to co-administer a counteracting agent or otherwise treat the undesired effect. Similarly, even within a single cell type, undesirable biological effects can be determined at the molecular level. Thus, the effects of an agent on expression of other than the target gene can be ascertained and counteracted.

In another embodiment, the array can be used to monitor expression of one or more genes in the array with respect to time. For example, samples obtained from different time points can be probed with the array. Such analysis can identify and/or characterize the development of a 23566, 33489, or 57779-associated disease or disorder; and processes, such as a cellular transformation associated with a 23566, 33489, or 57779-associated disease or disorder. The method can also evaluate the treatment and/or progression of a 23566, 33489, or 57779-associated disease or disorder

The array is also useful for ascertaining differential expression patterns of one or more genes in normal and abnormal cells. This provides a battery of genes (e.g., including 23566, 33489, or 57779) that could serve as a molecular target for diagnosis or therapeutic intervention.

In another aspect, the invention features an array having a plurality of addresses. Each address of the plurality includes a unique polypeptide. At least one address of the plurality has disposed thereon a 23566, 33489, or 57779 polypeptide or fragment thereof. Methods of producing polypeptide arrays are described in the art, e.g., in De Wildt et al. (2000). Nature Biotech. 18, 989-994; Lueking et al. (1999). Anal. Biochem. 270, 103-111; Ge, H. (2000). Nucleic Acids Res. 28, e3, I-VII; MacBeath, G., and Schreiber, S. L. (2000). Science 289, 1760-1763; and WO 99/51773A1. In a preferred embodiment, each addresses of the plurality has disposed thereon a polypeptide at least 60, 70, 80, 85, 90, 95 or 99% identical to a 23566, 33489, or 57779 polypeptide or fragment thereof. For example, multiple variants of a 23566, 33489, or 57779 polypeptide (e.g., encoded by allelic variants, site-directed mutants, random mutants, or combinatorial mutants) can be disposed at individual addresses of the plurality. Addresses in addition to the address of the plurality can be disposed on the array.

The polypeptide array can be used to detect a 23566, 33489, or 57779 binding compound, e.g., an antibody in a sample from a subject with specificity for a 23566, 33489, or 57779 polypeptide or the presence of a 23566, 33489, or 57779-binding protein or ligand.

The array is also useful for ascertaining the effect of the expression of a gene on the expression of other genes in the same cell or in different cells (e.g., ascertaining the effect of 23566, 33489, or 57779 expression on the expression of other genes). This provides, for example, for a selection of alternate molecular targets for therapeutic intervention if the ultimate or downstream target cannot be regulated.

In another aspect, the invention features a method of analyzing a plurality of probes. The method is useful, e.g., for analyzing gene expression. The method includes: providing a two dimensional array having a plurality of addresses, each address of the plurality being positionally distinguishable from each other address of the plurality having a unique capture probe, e.g., wherein the capture probes are from a cell or subject which express 23566, 33489, or 57779 or from a cell or subject in which a 23566, 33489, or 57779 mediated response has been elicited, e.g., by contact of the cell with 23566, 33489, or 57779 nucleic acid or protein, or administration to the cell or subject 23566, 33489, or 57779 nucleic acid or protein; providing a two dimensional array having a plurality of addresses, each address of the plurality being positionally distinguishable from each other address of the plurality, and each address of the plurality having a unique capture probe, e.g., wherein the capture probes are from a cell or subject which does not express 23566, 33489, or 57779 (or does not express as highly as in the case of the 23566, 33489, or 57779 positive plurality of capture probes) or from a cell or subject which in which a 23566, 33489, or 57779 mediated response has not been elicited (or has been elicited to a lesser extent than in the first sample); contacting the array with one or more inquiry probes (which is preferably other than a 23566, 33489, or 57779 nucleic acid, polypeptide, or antibody), and thereby evaluating the plurality of capture probes. Binding, e.g., in the case of a nucleic acid, hybridization with a capture probe at an address of the plurality, is detected, e.g., by signal generated from a label attached to the nucleic acid, polypeptide, or antibody.

In another aspect, the invention features a method of analyzing a plurality of probes or a sample. The method is useful, e.g., for analyzing gene expression. The method includes: providing a two dimensional array having a plurality of addresses, each address of the plurality being positionally distinguishable from each other address of the plurality having a unique capture probe, contacting the array with a first sample from a cell or subject which express or mis-express 23566, 33489, or 57779 or from a cell or subject in which a 23566, 33489, or 57779-mediated response has been elicited, e.g., by contact of the cell with 23566, 33489, or 57779 nucleic acid or protein, or administration to the cell or subject 23566, 33489, or 57779 nucleic acid or protein; providing a two dimensional array having a plurality of addresses, each address of the plurality being positionally distinguishable from each other address of the plurality, and each address of the plurality having a unique capture probe, and contacting the array with a second sample from a cell or subject which does not express 23566, 33489, or 57779 (or does not express as highly as in the case of the 23566, 33489, or 57779 positive plurality of capture probes) or from a cell or subject which in which a 23566, 33489, or 57779 mediated response has not been elicited (or has been elicited to a lesser extent than in the first sample); and comparing the binding of the first sample with the binding of the second sample. Binding, e.g., in the case of a nucleic acid, hybridization with a capture probe at an address of the plurality, is detected, e.g., by signal generated from a label attached to the nucleic acid, polypeptide, or antibody. The same array can be used for both samples or different arrays can be used. If different arrays are used the plurality of addresses with capture probes should be present on both arrays.

In another aspect, the invention features a method of analyzing 23566, 33489, or 57779, e.g., analyzing structure, function, or relatedness to other nucleic acid or amino acid sequences. The method includes: providing a 23566, 33489, or 57779 nucleic acid or amino acid sequence; comparing the 23566, 33489, or 57779 sequence with one or more preferably a plurality of sequences from a collection of sequences, e.g., a nucleic acid or protein sequence database; to thereby analyze 23566, 33489, or 57779.

Detection of 23566, 33489, and 57779 Variations or Mutations

The methods of the invention can also be used to detect genetic alterations in a 23566, 33489, or 57779 gene, thereby determining if a subject with the altered gene is at risk for a disorder characterized by misregulation in 23566, 33489, or 57779 protein activity or nucleic acid expression, such as an inflammatory disorder, neurological disorder, cardiovascular disorder (e.g., disorder of the heart and/or blood vessels), blood clotting disorder, or cellular proliferation and/or differentiation disorder. In preferred embodiments, the methods include detecting, in a sample from the subject, the presence or absence of a genetic alteration characterized by at least one of an alteration affecting the integrity of a gene encoding a 23566, 33489, or 57779-protein, or the mis-expression of the 23566, 33489, or 57779 gene. For example, such genetic alterations can be detected by ascertaining the existence of at least one of 1) a deletion of one or more nucleotides from a 23566, 33489, or 57779 gene; 2) an addition of one or more nucleotides to a 23566, 33489, or 57779 gene; 3) a substitution of one or more nucleotides of a 23566, 33489, or 57779 gene, 4) a chromosomal rearrangement of a 23566, 33489, or 57779 gene; 5) an alteration in the level of a messenger RNA transcript of a 23566, 33489, or 57779 gene, 6) aberrant modification of a 23566, 33489, or 57779 gene, such as of the methylation pattern of the genomic DNA, 7) the presence of a non-wild type splicing pattern of a messenger RNA transcript of a 23566, 33489, or 57779 gene, 8) a non-wild type level of a 23566, 33489, or 57779-protein, 9) allelic loss of a 23566, 33489, or 57779 gene, and 10) inappropriate post-translational modification of a 23566, 33489, or 57779-protein.

An alteration can be detected without a probe/primer in a polymerase chain reaction, such as anchor PCR or RACE PCR, or, alternatively, in a ligation chain reaction (LCR), the latter of which can be particularly useful for detecting point mutations in the 23566, 33489, or 57779-gene. This method can include the steps of collecting a sample of cells from a subject, isolating nucleic acid (e.g., genomic, mRNA or both) from the sample, contacting the nucleic acid sample with one or more primers which specifically hybridize to a 23566, 33489, or 57779 gene under conditions such that hybridization and amplification of the 23566, 33489, or 57779-gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein. Alternatively, other amplification methods described herein or known in the art can be used.

In another embodiment, mutations in a 23566, 33489, or 57779 gene from a sample cell can be identified by detecting alterations in restriction enzyme cleavage patterns. For example, sample and control DNA is isolated, amplified (optionally), digested with one or more restriction endonucleases, and fragment length sizes are determined, e.g., by gel electrophoresis and compared. Differences in fragment length sizes between sample and control DNA indicates mutations in the sample DNA. Moreover, the use of sequence specific ribozymes (see, for example, U.S. Pat. No. 5,498,531) can be used to score for the presence of specific mutations by development or loss of a ribozyme cleavage site.

In other embodiments, genetic mutations in 23566, 33489, or 57779 can be identified by hybridizing a sample and control nucleic acids, e.g., DNA or RNA, two-dimensional arrays, e.g., chip based arrays. Such arrays include a plurality of addresses, each of which is positionally distinguishable from the other. A different probe is located at each address of the plurality. A probe can be complementary to a region of a 23566, 33489, or 57779 nucleic acid or a putative variant (e.g., allelic variant) thereof. A probe can have one or more mismatches to a region of a 23566, 33489, or 57779 nucleic acid (e.g., a destabilizing mismatch). The arrays can have a high density of addresses, e.g., can contain hundreds or thousands of oligonucleotides probes (Cronin, M. T. et al. (1996) Human Mutation 7: 244-255; Kozal, M. J. et al. (1996) Nature Medicine 2: 753-759). For example, genetic mutations in 23566, 33489, or 57779 can be identified in two-dimensional arrays containing light-generated DNA probes as described in Cronin, M. T. et al. supra. Briefly, a first hybridization array of probes can be used to scan through long stretches of DNA in a sample and control to identify base changes between the sequences by making linear arrays of sequential overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the 23566, 33489, or 57779 gene and detect mutations by comparing the sequence of the sample 23566, 33489, or 57779 with the corresponding wild-type (control) sequence. Automated sequencing procedures can be utilized when performing the diagnostic assays ((1995) Biotechniques 19:448), including sequencing by mass spectrometry.

Other methods for detecting mutations in the 23566, 33489, or 57779 gene include methods in which protection from cleavage agents is used to detect mismatched bases in RNA/RNA or RNA/DNA heteroduplexes (Myers et al. (1985) Science 230:1242; Cotton et al. (1988) Proc. Natl. Acad Sci USA 85:4397; Saleeba et al. (1992) Methods Enzymol. 217:286-295).

In still another embodiment, the mismatch cleavage reaction employs one or more proteins that recognize mismatched base pairs in double-stranded DNA (so called “DNA mismatch repair” enzymes) in defined systems for detecting and mapping point mutations in 23566, 33489, or 57779 cDNAs obtained from samples of cells. For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1662; U.S. Pat. No. 5,459,039).

In other embodiments, alterations in electrophoretic mobility will be used to identify mutations in 23566, 33489, or 57779 genes. For example, single strand conformation polymorphism (SSCP) may be used to detect differences in electrophoretic mobility between mutant and wild type nucleic acids (Orita et al. (1989) Proc Natl. Acad. Sci USA: 86:2766, see also Cotton (1993) Mutat. Res. 285:125-144; and Hayashi (1992) Genet. Anal. Tech. Appl. 9:73-79). Single-stranded DNA fragments of sample and control 23566, 33489, or 57779 nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. (1991) Trends Genet 7:5).

In yet another embodiment, the movement of mutant or wild-type fragments in polyacrylamide gels containing a gradient of denaturant is assayed using denaturing gradient gel electrophoresis (DGGE) (Myers et al. (1985) Nature 313:495). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner (1987) Biophys Chem 265:12753).

Examples of other techniques for detecting point mutations include, but are not limited to, selective oligonucleotide hybridization, selective amplification, or selective primer extension (Saiki et al. (1986) Nature 324:163); Saiki et al. (1989) Proc. Natl Acad. Sci USA 86:6230). A further method of detecting point mutations is the chemical ligation of oligonucleotides as described in Xu et al. ((2001) Nature Biotechnol. 19:148). Adjacent oligonucleotides, one of which selectively anneals to the query site, are ligated together if the nucleotide at the query site of the sample nucleic acid is complementary to the query oligonucleotide; ligation can be monitored, e.g., by fluorescent dyes coupled to the oligonucleotides.

Alternatively, allele specific amplification technology that depends on selective PCR amplification may be used in conjunction with the instant invention. Oligonucleotides used as primers for specific amplification may carry the mutation of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent, or reduce polymerase extension (Prossner (1993) Tibtech 11:238). In addition it may be desirable to introduce a novel restriction site in the region of the mutation to create cleavage-based detection (Gasparini et al. (1992) Mol. Cell Probes 6:1). It is anticipated that in certain embodiments amplification may also be performed using Taq ligase for amplification (Barany (1991) Proc. Natl. Acad. Sci USA 88:189). In such cases, ligation will occur only if there is a perfect match at the 3′ end of the 5′ sequence making it possible to detect the presence of a known mutation at a specific site by looking for the presence or absence of amplification.

In another aspect, the invention features a set of oligonucleotides. The set includes a plurality of oligonucleotides, each of which is at least partially complementary (e.g., at least 50%, 60%, 70%, 80%, 90%, 92%, 95%, 97%, 98%, or 99% complementary) to a 23566, 33489, or 57779 nucleic acid.

In a preferred embodiment the set includes a first and a second oligonucleotide. The first and second oligonucleotide can hybridize to the same or to different locations of SEQ ID NO:73, SEQ ID NO:76, or SEQ ID NO:79, or the complement of SEQ ID NO:73, SEQ ID NO:76, or SEQ ID NO:79. Different locations can be different but overlapping, or non-overlapping on the same strand. The first and second oligonucleotide can hybridize to sites on the same or on different strands.

The set can be useful, e.g., for identifying SNP's, or identifying specific alleles of 23566, 33489, or 57779. In a preferred embodiment, each oligonucleotide of the set has a different nucleotide at an interrogation position. In one embodiment, the set includes two oligonucleotides, each complementary to a different allele at a locus, e.g., a biallelic or polymorphic locus.

In another embodiment, the set includes four oligonucleotides, each having a different nucleotide (e.g., adenine, guanine, cytosine, or thymidine) at the interrogation position. The interrogation position can be a SNP or the site of a mutation. In another preferred embodiment, the oligonucleotides of the plurality are identical in sequence to one another (except for differences in length). The oligonucleotides can be provided with differential labels, such that an oligonucleotide that hybridizes to one allele provides a signal that is distinguishable from an oligonucleotide that hybridizes to a second allele. In still another embodiment, at least one of the oligonucleotides of the set has a nucleotide change at a position in addition to a query position, e.g., a destabilizing mutation to decrease the Tₘ of the oligonucleotide. In another embodiment, at least one oligonucleotide of the set has a non-natural nucleotide, e.g., inosine. In a preferred embodiment, the oligonucleotides are attached to a solid support, e.g., to different addresses of an array or to different beads or nanoparticles.

In a preferred embodiment the set of oligo nucleotides can be used to specifically amplify, e.g., by PCR, or detect, a 23566, 33489, or 57779 nucleic acid.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving a 23566, 33489, or 57779 gene.

Use of 23566, 33489, or 57779 Molecules as Surrogate Markers

The 23566, 33489, or 57779 molecules of the invention are also useful as markers of disorders or disease states, as markers for precursors of disease states, as markers for predisposition of disease states, as markers of drug activity, or as markers of the pharmacogenomic profile of a subject. Using the methods described herein, the presence, absence and/or quantity of the 23566, 33489, or 57779 molecules of the invention may be detected, and may be correlated with one or more biological states in vivo. For example, the 23566, 33489, or 57779 molecules of the invention may serve as surrogate markers for one or more disorders or disease states or for conditions leading up to disease states. As used herein, a “surrogate marker” is an objective biochemical marker which correlates with the absence or presence of a disease or disorder, or with the progression of a disease or disorder (e.g., with the presence or absence of a tumor). The presence or quantity of such markers is independent of the disease. Therefore, these markers may serve to indicate whether a particular course of treatment is effective in lessening a disease state or disorder. Surrogate markers are of particular use when the presence or extent of a disease state or disorder is difficult to assess through standard methodologies (e.g., early stage tumors), or when an assessment of disease progression is desired before a potentially dangerous clinical endpoint is reached (e.g., an assessment of cardiovascular disease may be made using cholesterol levels as a surrogate marker, and an analysis of HIV infection may be made using HIV RNA levels as a surrogate marker, well in advance of the undesirable clinical outcomes of myocardial infarction or fully-developed AIDS). Examples of the use of surrogate markers in the art include: Koomen et al. (2000) J. Mass. Spectrom. 35: 258-264; and James (1994) AIDS Treatment News Archive 209.

The 23566, 33489, or 57779 molecules of the invention are also useful as pharmacodynamic markers. As used herein, a “pharmacodynamic marker” is an objective biochemical marker which correlates specifically with drug effects. The presence or quantity of a pharmacodynamic marker is not related to the disease state or disorder for which the drug is being administered; therefore, the presence or quantity of the marker is indicative of the presence or activity of the drug in a subject. For example, a pharmacodynamic marker may be indicative of the concentration of the drug in a biological tissue, in that the marker is either expressed or transcribed or not expressed or transcribed in that tissue in relationship to the level of the drug. In this fashion, the distribution or uptake of the drug may be monitored by the pharmacodynamic marker. Similarly, the presence or quantity of the pharmacodynamic marker may be related to the presence or quantity of the metabolic product of a drug, such that the presence or quantity of the marker is indicative of the relative breakdown rate of the drug in vivo. Pharmacodynamic markers are of particular use in increasing the sensitivity of detection of drug effects, particularly when the drug is administered in low doses. Since even a small amount of a drug may be sufficient to activate multiple rounds of marker (e.g., a 23566, 33489, or 57779 marker) transcription or expression, the amplified marker may be in a quantity which is more readily detectable than the drug itself. Also, the marker may be more easily detected due to the nature of the marker itself; for example, using the methods described herein, anti-23566, 33489, or 57779 antibodies may be employed in an immune-based detection system for a 23566, 33489, or 57779 protein marker, or 23566, 33489, or 57779-specific radiolabeled probes may be used to detect a 23566, 33489, or 57779 mRNA marker. Furthermore, the use of a pharmacodynamic marker may offer mechanism-based prediction of risk due to drug treatment beyond the range of possible direct observations. Examples of the use of pharmacodynamic markers in the art include: Matsuda et al. U.S. Pat. No. 6,033,862; Hattis et al. (1991) Env. Health Perspect. 90: 229-238; Schentag (1999) Am. J. Health-Syst. Pharm. 56 Suppl. 3: S21-S24; and Nicolau (1999) Am, J Health-Syst. Pharm. 56 Suppl. 3: S16-S20.

The 23566, 33489, or 57779 molecules of the invention are also useful as pharmacogenomic markers. As used herein, a “pharmacogenomic marker” is an objective biochemical marker which correlates with a specific clinical drug response or susceptibility in a subject (see, e.g., McLeod et al. (1999) Eur. J. Cancer 35:1650-1652). The presence or quantity of the pharmacogenomic marker is related to the predicted response of the subject to a specific drug or class of drugs prior to administration of the drug. By assessing the presence or quantity of one or more pharmacogenomic markers in a subject, a drug therapy which is most appropriate for the subject, or which is predicted to have a greater degree of success, may be selected. For example, based on the presence or quantity of RNA, or protein (e.g., 23566, 33489, or 57779 protein or RNA) for specific tumor markers in a subject, a drug or course of treatment may be selected that is optimized for the treatment of the specific tumor likely to be present in the subject. Similarly, the presence or absence of a specific sequence mutation in 23566, 33489, or 57779 DNA may correlate 23566, 33489, or 57779 drug response. The use of pharmacogenomic markers therefore permits the application of the most appropriate treatment for each subject without having to administer the therapy.

Pharmaceutical Compositions of 23566, 33489, and 57779

The nucleic acid and polypeptides, fragments thereof, as well as anti-23566, 33489, or 57779 antibodies (also referred to herein as “active compounds”) of the invention can be incorporated into pharmaceutical compositions. Such compositions typically include the nucleic acid molecule, protein, or antibody and a pharmaceutically acceptable carrier. As used herein the language “pharmaceutically acceptable carrier” includes solvents, dispersion media, coatings, antibacterial and antifungal agents, isotonic and absorption delaying agents, and the like, compatible with pharmaceutical administration. Supplementary active compounds can also be incorporated into the compositions.

A pharmaceutical composition is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor ELTM (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

As defined herein, a therapeutically effective amount of protein or polypeptide (i.e., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The protein or polypeptide can be administered one time per week for between about 1 to 10 weeks, preferably between 2 to 8 weeks, more preferably between about 3 to 7 weeks, and even more preferably for about 4, 5, or 6 weeks. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of a protein, polypeptide, or antibody can include a single treatment or, preferably, can include a series of treatments.

For antibodies, the preferred dosage is 0.1 mg/kg of body weight (generally 10 mg/kg to 20 mg/kg). If the antibody is to act in the brain, a dosage of 50 mg/kg to 100 mg/kg is usually appropriate. Generally, partially human antibodies and fully human antibodies have a longer half-life within the human body than other antibodies. Accordingly, lower dosages and less frequent administration is often possible. Modifications such as lipidation can be used to stabilize antibodies and to enhance uptake and tissue penetration (e.g., into the brain). A method for lipidation of antibodies is described by Cruikshank et al. ((1997) J. Acquired Immune Deficiency Syndromes and Human Retrovirology 14:193).

The present invention encompasses agents which modulate expression or activity. An agent may, for example, be a small molecule. For example, such small molecules include, but are not limited to, peptides, peptidomimetics (e.g., peptoids), amino acids, amino acid analogs, polynucleotides, polynucleotide analogs, nucleotides, nucleotide analogs, organic or inorganic compounds (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

Exemplary doses include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram. It is furthermore understood that appropriate doses of a small molecule depend upon the potency of the small molecule with respect to the expression or activity to be modulated. When one or more of these small molecules is to be administered to an animal (e.g., a human) in order to modulate expression or activity of a polypeptide or nucleic acid of the invention, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

An antibody (or fragment thereof) may be conjugated to a therapeutic moiety such as a cytotoxin, a therapeutic agent or a radioactive ion. A cytotoxin or cytotoxic agent includes any agent that is detrimental to cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, puromycin, maytansinoids, e.g., maytansinol (see U.S. Pat. No. 5,208,020), CC-1065 (see U.S. Pat. Nos. 5,475,092, 5,585,499, 5,846,545) and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, CC-1065, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine, vinblastine, taxol and maytansinoids). Radioactive ions include, but are not limited to iodine, yttrium and praseodymium.

The conjugates of the invention can be used for modifying a given biological response, the drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, α-interferon, β-interferon, nerve growth factor, platelet derived growth factor, tissue plasminogen activator; or, biological response modifiers such as, for example, lymphokines, interleukin-1 (“IL-1”), interleukin-2 (“IL-2”), interleukin-6 (“IL-6”), granulocyte macrophase colony stimulating factor (“GM-CSF”), granulocyte colony stimulating factor (“G-CSF”), or other growth factors. Alternatively, an antibody can be conjugated to a second antibody to form an antibody heteroconjugate as described by Segal in U.S. Pat. No. 4,676,980.

The nucleic acid molecules of the invention can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

The pharmaceutical compositions can be included in a container, pack, or dispenser together with instructions for administration.

Methods of Treatment for 23566, 33489, and 57779

The present invention provides for both prophylactic and therapeutic methods of treating a subject at risk of (or susceptible to) a disorder or having a disorder associated with aberrant or unwanted 23566, 33489, or 57779 expression or activity. As used herein, the term “treatment” is defined as the application or administration of a therapeutic agent to a patient, or application or administration of a therapeutic agent to an isolated tissue or cell line from a patient, who has a disease, a symptom of disease or a predisposition toward a disease, with the purpose to cure, heal, alleviate, relieve, alter, remedy, ameliorate, improve or affect the disease, the symptoms of disease or the predisposition toward disease. A therapeutic agent includes, but is not limited to, small molecules, peptides, antibodies, ribozymes and antisense oligonucleotides.

With regards to both prophylactic and therapeutic methods of treatment, such treatments may be specifically tailored or modified, based on knowledge obtained from the field of pharmacogenomics. “Pharmacogenomics”, as used herein, refers to the application of genomics technologies such as gene sequencing, statistical genetics, and gene expression analysis to drugs in clinical development and on the market. More specifically, the term refers the study of how a patient's genes determine his or her response to a drug (e.g., a patient's “drug response phenotype”, or “drug response genotype”.) Thus, another aspect of the invention provides methods for tailoring an individual's prophylactic or therapeutic treatment with either the 23566, 33489, or 57779 molecules of the present invention or 23566, 33489, or 57779 modulators according to that individual's drug response genotype. Pharmacogenomics allows a clinician or physician to target prophylactic or therapeutic treatments to patients who will most benefit from the treatment and to avoid treatment of patients who will experience toxic drug-related side effects.

In one aspect, the invention provides a method for preventing in a subject, a disease or condition associated with an aberrant or unwanted 23566, 33489, or 57779 expression or activity, by administering to the subject a 23566, 33489, or 57779 or an agent which modulates 23566, 33489, or 57779 expression or at least one 23566, 33489, or 57779 activity. Subjects at risk for a disease which is caused or contributed to by aberrant or unwanted 23566, 33489, or 57779 expression or activity can be identified by, for example, any or a combination of diagnostic or prognostic assays as described herein. Administration of a prophylactic agent can occur prior to the manifestation of symptoms characteristic of the 23566, 33489, or 57779 aberrance, such that a disease or disorder is prevented or, alternatively, delayed in its progression. Depending on the type of 23566, 33489, or 57779 aberrance, for example, a 23566, 33489, or 57779, 23566, 33489, or 57779 agonist or 23566, 33489, or 57779 antagonist agent can be used for treating the subject. The appropriate agent can be determined based on screening assays described herein.

It is possible that some 23566, 33489, or 57779 disorders can be caused, at least in part, by an abnormal level of gene product, or by the presence of a gene product exhibiting abnormal activity. As such, the reduction in the level and/or activity of such gene products would bring about the amelioration of disorder symptoms.

The 23566, 33489, or 57779 molecules can act as novel diagnostic targets and therapeutic agents for controlling one or more disorder, e.g., digestive disorders (e.g., disorders of the small intestine), disorders of the pancreas, inflammatory disorders, neurological disorders, hormonal disorders/imbalances, cardiovascular disorders (e.g., disorders of the heart and/or blood vessels), apoptotic disorders, red blood cell disorders (e.g., blood clotting disorders, anemias), viral disorders, cell engulfiment disorders, or cellular adhesion, proliferation, and/or differentiation disorders, as discussed above. In addition, the 23566, 33489, or 57779 molecules of the invention can act as novel diagnostic targets and therapeutic agents for controlling disorders associated with bone metabolism, liver disorders, pain disorders, or metabolic disorders.

Aberrant expression and/or activity of 23566, 33489, or 57779 molecules may mediate disorders associated with bone metabolism. “Bone metabolism” refers to direct or indirect effects in the formation or degeneration of bone structures, e.g., bone formation, bone resorption, etc., which may ultimately affect the concentrations in serum of calcium and phosphate. This term also includes activities mediated by 23566, 33489, or 57779 molecules effects in bone cells, e.g. osteoclasts and osteoblasts, that may in turn result in bone formation and degeneration. For example, 23566, 33489, or 57779 molecules may support different activities of bone resorbing osteoclasts such as the stimulation of differentiation of monocytes and mononuclear phagocytes into osteoclasts. Accordingly, 23566, 33489, or 57779 molecules that modulate the production of bone cells can influence bone formation and degeneration, and thus may be used to treat bone disorders. Examples of such disorders include, but are not limited to, osteoporosis, osteodystrophy, osteomalacia, rickets, osteitis fibrosa cystica, renal osteodystrophy, osteosclerosis, anti-convulsant treatment, osteopenia, fibrogenesis-imperfecta ossium, secondary hyperparathyrodism, hypoparathyroidism, hyperparathyroidism, cirrhosis, obstructive jaundice, drug induced metabolism, medullary carcinoma, chronic renal disease, rickets, sarcoidosis, glucocorticoid antagonism, malabsorption syndrome, steatorrhea, tropical sprue, idiopathic hypercalcemia and milk fever.

Disorders which may be treated or diagnosed by methods described herein include, but are not limited to, disorders associated with an accumulation in the liver of fibrous tissue, such as that resulting from an imbalance between production and degradation of the extracellular matrix accompanied by the collapse and condensation of preexisting fibers. The methods described herein can be used to diagnose or treat hepatocellular necrosis or injury induced by a wide variety of agents including processes which disturb homeostasis, such as an inflammatory process, tissue damage resulting from toxic injury or altered hepatic blood flow, and infections (e.g., bacterial, viral and parasitic). For example, the methods can be used for the early detection of hepatic injury, such as portal hypertension or hepatic fibrosis. In addition, the methods can be employed to detect liver fibrosis attributed to inborn errors of metabolism, for example, fibrosis resulting from a storage disorder such as Gaucher's disease (lipid abnormalities) or a glycogen storage disease, A1-antitrypsin deficiency; a disorder mediating the accumulation (e.g., storage) of an exogenous substance, for example, hemochromatosis (iron-overload syndrome) and copper storage diseases (Wilson's disease), disorders resulting in the accumulation of a toxic metabolite (e.g., tyrosinemia, fructosemia and galactosemia) and peroxisomal disorders (e.g., Zellweger syndrome). Additionally, the methods described herein may be useful for the early detection and treatment of liver injury associated with the administration of various chemicals or drugs, such as for example, methotrexate, isonizaid, oxyphenisatin, methyldopa, chlorpromazine, tolbutamide or alcohol, or which represents a hepatic manifestation of a vascular disorder such as obstruction of either the intrahepatic or extrahepatic bile flow or an alteration in hepatic circulation resulting, for example, from chronic heart failure, veno-occlusive disease, portal vein thrombosis or Budd-Chiari syndrome.

Additionally, 23566, 33489, or 57779 may play an important role in the regulation of metabolism or pain disorders. Diseases of metabolic imbalance include, but are not limited to, obesity, anorexia nervosa, cachexia, lipid disorders, and diabetes. Examples of pain disorders include, but are not limited to, pain response elicited during various forms of tissue injury, e.g., inflammation, infection, and ischemia, usually referred to as hyperalgesia (described in, for example, Fields, H. L. (1987) Pain, New York:McGraw-Hill); pain associated with musculoskeletal disorders, e.g., joint pain; tooth pain; headaches; pain associated with surgery; pain related to irritable bowel syndrome; or chest pain.

As discussed, successful treatment of 23566, 33489, or 57779 disorders can be brought about by techniques that serve to inhibit the expression or activity of target gene products. For example, compounds, e.g., an agent identified using an assays described above, that proves to exhibit negative modulatory activity, can be used in accordance with the invention to prevent and/or ameliorate symptoms of 23566, 33489, or 57779 disorders. Such molecules can include, but are not limited to peptides, phosphopeptides, small organic or inorganic molecules, or antibodies (including, for example, polyclonal, monoclonal, humanized, anti-idiotypic, chimeric or single chain antibodies, and Fab, F(ab′)₂ and Fab expression library fragments, scFV molecules, and epitope-binding fragments thereof).

Further, antisense and ribozyme molecules that inhibit expression of the target gene can also be used in accordance with the invention to reduce the level of target gene expression, thus effectively reducing the level of target gene activity. Still further, triple helix molecules can be utilized in reducing the level of target gene activity. Antisense, ribozyme and triple helix molecules are discussed above.

It is possible that the use of antisense, ribozyme, and/or triple helix molecules to reduce or inhibit mutant gene expression can also reduce or inhibit the transcription (triple helix) and/or translation (antisense, ribozyme) of mRNA produced by normal target gene alleles, such that the concentration of normal target gene product present can be lower than is necessary for a normal phenotype. In such cases, nucleic acid molecules that encode and express target gene polypeptides exhibiting normal target gene activity can be introduced into cells via gene therapy method. Alternatively, in instances in that the target gene encodes an extracellular protein, it can be preferable to co-administer normal target gene protein into the cell or tissue in order to maintain the requisite level of cellular or tissue target gene activity.

Another method by which nucleic acid molecules may be utilized in treating or preventing a disease characterized by 23566, 33489, or 57779 expression is through the use of aptamer molecules specific for 23566, 33489, or 57779 protein. Aptamers are nucleic acid molecules having a tertiary structure which permits them to specifically bind to protein ligands (see, e.g., Osborne, et al. (1997) Curr. Opin. Chem Biol. 1: 5-9; and Patel, D. J. (1997) Curr Opin Chem Biol 1:32-46). Since nucleic acid molecules may in many cases be more conveniently introduced into target cells than therapeutic protein molecules may be, aptamers offer a method by which 23566, 33489, or 57779 protein activity may be specifically decreased without the introduction of drugs or other molecules which may have pluripotent effects.

Antibodies can be generated that are both specific for target gene product and that reduce target gene product activity. Such antibodies may, therefore, by administered in instances whereby negative modulatory techniques are appropriate for the treatment of 23566, 33489, or 57779 disorders. For a description of antibodies, see the Antibody section above.

In circumstances wherein injection of an animal or a human subject with a 23566, 33489, or 57779 protein or epitope for stimulating antibody production is harmful to the subject, it is possible to generate an immune response against 23566, 33489, or 57779 through the use of anti-idiotypic antibodies (see, for example, Herlyn, D. (1999) Ann Med 31:66-78; and Bhattacharya-Chattejee, M., and Foon, K. A. (1998) Cancer Treat Res. 94:51-68). If an anti-idiotypic antibody is introduced into a mammal or human subject, it should stimulate the production of anti-anti-idiotypic antibodies, which should be specific to the 23566, 33489, or 57779 protein. Vaccines directed to a disease characterized by 23566, 33489, or 57779 expression may also be generated in this fashion.

In instances where the target antigen is intracellular and whole antibodies are used, internalizing antibodies may be preferred. Lipofectin or liposomes can be used to deliver the antibody or a fragment of the Fab region that binds to the target antigen into cells. Where fragments of the antibody are used, the smallest inhibitory fragment that binds to the target antigen is preferred. For example, peptides having an amino acid sequence corresponding to the Fv region of the antibody can be used. Alternatively, single chain neutralizing antibodies that bind to intracellular target antigens can also be administered. Such single chain antibodies can be administered, for example, by expressing nucleotide sequences encoding single-chain antibodies within the target cell population (see e.g., Marasco et al. (1993) Proc. Natl. Acad. Sci. USA 90:7889-7893).

The identified compounds that inhibit target gene expression, synthesis and/or activity can be administered to a patient at therapeutically effective doses to prevent, treat or ameliorate 23566, 33489, or 57779 disorders. A therapeutically effective dose refers to that amount of the compound sufficient to result in amelioration of symptoms of the disorders. Toxicity and therapeutic efficacy of such compounds can be determined by standard pharmaceutical procedures as described above.

The data obtained from the cell culture assays and animal studies can be used in formulating a range of dosage for use in humans. The dosage of such compounds lies preferably within a range of circulating concentrations that include the ED₅₀ with little or no toxicity. The dosage can vary within this range depending upon the dosage form employed and the route of administration utilized. For any compound used in the method of the invention, the therapeutically effective dose can be estimated initially from cell culture assays. A dose can be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma can be measured, for example, by high performance liquid chromatography.

Another example of determination of effective dose for an individual is the ability to directly assay levels of “free” and “bound” compound in the serum of the test subject. Such assays may utilize antibody mimics and/or “biosensors” that have been created through molecular imprinting techniques. The compound which is able to modulate 23566, 33489, or 57779 activity is used as a template, or “imprinting molecule”, to spatially organize polymerizable monomers prior to their polymerization with catalytic reagents. The subsequent removal of the imprinted molecule leaves a polymer matrix which contains a repeated “negative image” of the compound and is able to selectively rebind the molecule under biological assay conditions. A detailed review of this technique can be seen in Ansell, R. J. et al. (1996) Current Opinion in Biotechnology 7:89-94 and in Shea, K. J. (1994) Trends in Polymer Science 2:166-173. Such “imprinted” affinity matrixes are amenable to ligand-binding assays, whereby the immobilized monoclonal antibody component is replaced by an appropriately imprinted matrix. An example of the use of such matrixes in this way can be seen in Vlatakis, G. et al. (1993) Nature 361:645-647. Through the use of isotope-labeling, the “free” concentration of compound which modulates the expression or activity of 23566, 33489, or 57779 can be readily monitored and used in calculations of IC₅₀.

Such “imprinted” affinity matrixes can also be designed to include fluorescent groups whose photon-emitting properties measurably change upon local and selective binding of target compound. These changes can be readily assayed in real time using appropriate fiberoptic devices, in turn allowing the dose in a test subject to be quickly optimized based on its individual IC50. An rudimentary example of such a “biosensor” is discussed in Kriz, D. et al. (1995) Analytical Chemistry 67:2142-2144.

Another aspect of the invention pertains to methods of modulating 23566, 33489, or 57779 expression or activity for therapeutic purposes. Accordingly, in an exemplary embodiment, the modulatory method of the invention involves contacting a cell with a 23566, 33489, or 57779 or agent that modulates one or more of the activities of 23566, 33489, or 57779 protein activity associated with the cell. An agent that modulates 23566, 33489, or 57779 protein activity can be an agent as described herein, such as a nucleic acid or a protein, a naturally-occurring target molecule of a 23566, 33489, or 57779 protein (e.g., a 23566, 33489, or 57779 substrate or receptor), a 23566, 33489, or 57779 antibody, a 23566, 33489, or 57779 agonist or antagonist, a peptidomimetic of a 23566, 33489, or 57779 agonist or antagonist, or other small molecule.

In one embodiment, the agent stimulates one or 23566, 33489, or 57779 activities. Examples of such stimulatory agents include active 23566, 33489, or 57779 protein and a nucleic acid molecule encoding 23566, 33489, or 57779. In another embodiment, the agent inhibits one or more 23566, 33489, or 57779 activities. Examples of such inhibitory agents include antisense 23566, 33489, or 57779 nucleic acid molecules, anti-23566, 33489, or 57779 antibodies, and 23566, 33489, or 57779 inhibitors. These modulatory methods can be performed in vitro (e.g., by culturing the cell with the agent) or, alternatively, in vivo (e.g., by administering the agent to a subject). As such, the present invention provides methods of treating an individual afflicted with a disease or disorder characterized by aberrant or unwanted expression or activity of a 23566, 33489, or 57779 protein or nucleic acid molecule. In one embodiment, the method involves administering an agent (e.g., an agent identified by a screening assay described herein), or combination of agents that modulates (e.g., up regulates or down regulates) 23566, 33489, or 57779 expression or activity. In another embodiment, the method involves administering a 23566, 33489, or 57779 protein or nucleic acid molecule as therapy to compensate for reduced, aberrant, or unwanted 23566, 33489, or 57779 expression or activity.

Stimulation of 23566, 33489, or 57779 activity is desirable in situations in which 23566, 33489, or 57779 is abnormally downregulated and/or in which increased 23566, 33489, or 57779 activity is likely to have a beneficial effect. For example, stimulation of 23566, 33489, or 57779 activity is desirable in situations in which a 23566, 33489, or 57779 is downregulated and/or in which increased 23566, 33489, or 57779 activity is likely to have a beneficial effect. Likewise, inhibition of 23566, 33489, or 57779 activity is desirable in situations in which 23566, 33489, or 57779 is abnormally upregulated and/or in which decreased 23566, 33489, or 57779 activity is likely to have a beneficial effect.

23566, 33489, and 57779 Pharmacogenomics

The 23566, 33489, or 57779 molecules of the present invention, as well as agents, or modulators which have a stimulatory or inhibitory effect on 23566, 33489, or 57779 activity (e.g., 23566, 33489, or 57779 gene expression) as identified by a screening assay described herein can be administered to individuals to treat (prophylactically or therapeutically) 23566, 33489, or 57779 associated disorders (e.g., inflammatory disorders, neurological disorders, cardiovascular disorders (e.g., disorders of the heart and/or blood vessels), blood clotting disorders, or cellular proliferation and/or differentiation disorders) associated with aberrant or unwanted 23566, 33489, or 57779 activity. In conjunction with such treatment, pharmacogenomics (i.e., the study of the relationship between an individual's genotype and that individual's response to a foreign compound or drug) may be considered. Differences in metabolism of therapeutics can lead to severe toxicity or therapeutic failure by altering the relation between dose and blood concentration of the pharmacologically active drug. Thus, a physician or clinician may consider applying knowledge obtained in relevant pharmacogenomics studies in determining whether to administer a 23566, 33489, or 57779 molecule or 23566, 33489, or 57779 modulator as well as tailoring the dosage and/or therapeutic regimen of treatment with a 23566, 33489, or 57779 molecule or 23566, 33489, or 57779 modulator.

Pharmacogenomics deals with clinically significant hereditary variations in the response to drugs due to altered drug disposition and abnormal action in affected persons. See, for example, Eichelbaum, M. et al. (1996) Clin. Exp. Pharmacol. Physiol. 23:983-985 and Linder, M. W. et al. (1997) Clin. Chem. 43:254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare genetic defects or as naturally-occurring polymorphisms. For example, glucose-6-phosphate dehydrogenase deficiency (G6PD) is a common inherited enzymopathy in which the main clinical complication is haemolysis after ingestion of oxidant drugs (anti-malarials, sulfonamides, analgesics, nitrofurans) and consumption of fava beans.

One pharmacogenomics approach to identifying genes that predict drug response, known as “a genome-wide association”, relies primarily on a high-resolution map of the human genome consisting of already known gene-related markers (e.g., a “bi-allelic” gene marker map which consists of 60,000-100,000 polymorphic or variable sites on the human genome, each of which has two variants.) Such a high-resolution genetic map can be compared to a map of the genome of each of a statistically significant number of patients taking part in a Phase II/III drug trial to identify markers associated with a particular observed drug response or side effect. Alternatively, such a high resolution map can be generated from a combination of some ten-million known single nucleotide polymorphisms (SNPs) in the human genome. As used herein, a “SNP” is a common alteration that occurs in a single nucleotide base in a stretch of DNA. For example, a SNP may occur once per every 1000 bases of DNA. A SNP may be involved in a disease process, however, the vast majority may not be disease-associated. Given a genetic map based on the occurrence of such SNPs, individuals can be grouped into genetic categories depending on a particular pattern of SNPs in their individual genome. In such a manner, treatment regimens can be tailored to groups of genetically similar individuals, taking into account traits that may be common among such genetically similar individuals.

Alternatively, a method termed the “candidate gene approach,” can be utilized to identify genes that predict drug response. According to this method, if a gene that encodes a drug's target is known (e.g., a 23566, 33489, or 57779 protein of the present invention), all common variants of that gene can be fairly easily identified in the population and it can be determined if having one version of the gene versus another is associated with a particular drug response.

Alternatively, a method termed the “gene expression profiling,” can be utilized to identify genes that predict drug response. For example, the gene expression of an animal dosed with a drug (e.g., a 23566, 33489, or 57779 molecule or 23566, 33489, or 57779 modulator of the present invention) can give an indication whether gene pathways related to toxicity have been turned on.

Information generated from more than one of the above pharmacogenomics approaches can be used to determine appropriate dosage and treatment regimens for prophylactic or therapeutic treatment of an individual. This knowledge, when applied to dosing or drug selection, can avoid adverse reactions or therapeutic failure and thus enhance therapeutic or prophylactic efficiency when treating a subject with a 23566, 33489, or 57779 molecule or 23566, 33489, or 57779 modulator, such as a modulator identified by one of the exemplary screening assays described herein.

The present invention further provides methods for identifying new agents, or combinations, that are based on identifying agents that modulate the activity of one or more of the gene products encoded by one or more of the 23566, 33489, or 57779 genes of the present invention, wherein these products may be associated with resistance of the cells to a therapeutic agent. Specifically, the activity of the proteins encoded by the 23566, 33489, or 57779 genes of the present invention can be used as a basis for identifying agents for overcoming agent resistance. By blocking the activity of one or more of the resistance proteins, target cells, e.g., human cells, will become sensitive to treatment with an agent that the unmodified target cells were resistant to.

Monitoring the influence of agents (e.g., drugs) on the expression or activity of a 23566, 33489, or 57779 protein can be applied in clinical trials. For example, the effectiveness of an agent determined by a screening assay as described herein to increase 23566, 33489, or 57779 gene expression, protein levels, or upregulate 23566, 33489, or 57779 activity, can be monitored in clinical trials of subjects exhibiting decreased 23566, 33489, or 57779 gene expression, protein levels, or downregulated 23566, 33489, or 57779 activity. Alternatively, the effectiveness of an agent determined by a screening assay to decrease 23566, 33489, or 57779 gene expression, protein levels, or downregulate 23566, 33489, or 57779 activity, can be monitored in clinical trials of subjects exhibiting increased 23566, 33489, or 57779 gene expression, protein levels, or upregulated 23566, 33489, or 57779 activity. In such clinical trials, the expression or activity of a 23566, 33489, or 57779 gene, and preferably, other genes that have been implicated in, for example, a 23566, 33489, or 57779-associated disorder can be used as a “read out” or markers of the phenotype of a particular cell.

23566, 33489, or 57779 Informatics

The sequence of a 23566, 33489, or 57779 molecule is provided in a variety of media to facilitate use thereof. A sequence can be provided as a manufacture, other than an isolated nucleic acid or amino acid molecule, which contains a 23566, 33489, or 57779. Such a manufacture can provide a nucleotide or amino acid sequence, e.g., an open reading frame, in a form which allows examination of the manufacture using means not directly applicable to examining the nucleotide or amino acid sequences, or a subset thereof, as they exists in nature or in purified form. The sequence information can include, but is not limited to, 23566, 33489, or 57779 full-length nucleotide and/or amino acid sequences, partial nucleotide and/or amino acid sequences, polymorphic sequences including single nucleotide polymorphisms (SNPs), epitope sequence, and the like. In a preferred embodiment, the manufacture is a machine-readable medium, e.g., a magnetic, optical, chemical or mechanical information storage device.

As used herein, “machine-readable media” refers to any medium that can be read and accessed directly by a machine, e.g., a digital computer or analogue computer. Non-limiting examples of a computer include a desktop PC, laptop, mainframe, server (e.g., a web server, network server, or server farm), handheld digital assistant, pager, mobile telephone, and the like. The computer can be stand-alone or connected to a communications network, e.g., a local area network (such as a VPN or intranet), a wide area network (e.g., an Extranet or the Internet), or a telephone network (e.g., a wireless, DSL, or ISDN network). Machine-readable media include, but are not limited to: magnetic storage media, such as floppy discs, hard disc storage medium, and magnetic tape; optical storage media such as CD-ROM; electrical storage media such as RAM, ROM, EPROM, EEPROM, flash memory, and the like; and hybrids of these categories such as magnetic/optical storage media.

A variety of data storage structures are available to a skilled artisan for creating a machine-readable medium having recorded thereon a nucleotide or amino acid sequence of the present invention. The choice of the data storage structure will generally be based on the means chosen to access the stored information. In addition, a variety of data processor programs and formats can be used to store the nucleotide sequence information of the present invention on computer readable medium. The sequence information can be represented in a word processing text file, formatted in commercially-available software such as WordPerfect and Microsoft Word, or represented in the form of an ASCII file, stored in a database application, such as DB2, Sybase, Oracle, or the like. The skilled artisan can readily adapt any number of data processor structuring formats (e.g., text file or database) in order to obtain computer readable medium having recorded thereon the nucleotide sequence information of the present invention.

In a preferred embodiment, the sequence information is stored in a relational database (such as Sybase or Oracle). The database can have a first table for storing sequence (nucleic acid and/or amino acid sequence) information. The sequence information can be stored in one field (e.g., a first column) of a table row and an identifier for the sequence can be store in another field (e.g., a second column) of the table row. The database can have a second table, e.g., storing annotations. The second table can have a field for the sequence identifier, a field for a descriptor or annotation text (e.g., the descriptor can refer to a functionality of the sequence, a field for the initial position in the sequence to which the annotation refers, and a field for the ultimate position in the sequence to which the annotation refers. Non-limiting examples for annotation to nucleic acid sequences include polymorphisms (e.g., SNP's) translational regulatory sites and splice junctions. Non-limiting examples for annotations to amino acid sequence include polypeptide domains, e.g., a domain described herein; active sites and other functional amino acids; and modification sites.

By providing the nucleotide or amino acid sequences of the invention in computer readable form, the skilled artisan can routinely access the sequence information for a variety of purposes. For example, one skilled in the art can use the nucleotide or amino acid sequences of the invention in computer readable form to compare a target sequence or target structural motif with the sequence information stored within the data storage means. A search is used to identify fragments or regions of the sequences of the invention which match a particular target sequence or target motif The search can be a BLAST search or other routine sequence comparison, e.g., a search described herein.

Thus, in one aspect, the invention features a method of analyzing 23566, 33489, or 57779, e.g., analyzing structure, function, or relatedness to one or more other nucleic acid or amino acid sequences. The method includes: providing a 23566, 33489, or 57779 nucleic acid or amino acid sequence; comparing the 23566, 33489, or 57779 sequence with a second sequence, e.g., one or more preferably a plurality of sequences from a collection of sequences, e.g., a nucleic acid or protein sequence database to thereby analyze 23566, 33489, or 57779. The method can be performed in a machine, e.g., a computer, or manually by a skilled artisan.

The method can include evaluating the sequence identity between a 23566, 33489, or 57779 sequence and a database sequence. The method can be performed by accessing the database at a second site, e.g., over the Internet.

As used herein, a “target sequence” can be any DNA or amino acid sequence of six or more nucleotides or two or more amino acids. A skilled artisan can readily recognize that the longer a target sequence is, the less likely a target sequence will be present as a random occurrence in the database. Typical sequence lengths of a target sequence are from about 10 to 100 amino acids or from about 30 to 300 nucleotide residues. However, it is well recognized that commercially important fragments, such as sequence fragments involved in gene expression and protein processing, may be of shorter length.

Computer software is publicly available which allows a skilled artisan to access sequence information provided in a computer readable medium for analysis and comparison to other sequences. A variety of known algorithms are disclosed publicly and a variety of commercially available software for conducting search means are and can be used in the computer-based systems of the present invention. Examples of such software include, but are not limited to, MacPattern (EMBL), BLASTN and BLASTX (NCBI).

Thus, the invention features a method of making a computer readable record of a sequence of a 23566, 33489, or 57779 sequence which includes recording the sequence on a computer readable matrix. In a preferred embodiment the record includes one or more of the following: identification of an ORF; identification of a domain, region, or site; identification of the start of transcription; identification of the transcription terminator; the full length amino acid sequence of the protein, or a mature form thereof; the 5′ end of the translated region.

In another aspect, the invention features, a method of analyzing a sequence. The method includes: providing a 23566, 33489, or 57779 sequence, or record, in machine-readable form; comparing a second sequence to the 23566, 33489, or 57779 sequence; thereby analyzing a sequence. Comparison can include comparing to sequences for sequence identity or determining if one sequence is included within the other, e.g., determining if the 23566, 33489, or 57779 sequence includes a sequence being compared. In a preferred embodiment the 23566, 33489, or 57779 or second sequence is stored on a first computer, e.g., at a first site and the comparison is performed, read, or recorded on a second computer, e.g., at a second site. E.g., the 23566, 33489, or 57779 or second sequence can be stored in a public or proprietary database in one computer, and the results of the comparison performed, read, or recorded on a second computer. In a preferred embodiment the record includes one or more of the following: identification of an ORF; identification of a domain, region, or site; identification of the start of transcription; identification of the transcription terminator; the full length amino acid sequence of the protein, or a mature form thereof; the 5′ end of the translated region.

In another aspect, the invention provides a machine-readable medium for holding instructions for performing a method for determining whether a subject has a 23566, 33489, or 57779-associated disease or disorder or a pre-disposition to a 23566, 33489, or 57779-associated disease or disorder, wherein the method comprises the steps of determining 23566, 33489, or 57779 sequence information associated with the subject and based on the 23566, 33489, or 57779 sequence information, determining whether the subject has a 23566, 33489, or 57779-associated disease or disorder or a pre-disposition to a 23566, 33489, or 57779-associated disease or disorder and/or recommending a particular treatment for the disease, disorder or pre-disease condition.

The invention further provides in an electronic system and/or in a network, a method for determining whether a subject has a 23566, 33489, or 57779-associated disease or disorder or a pre-disposition to a disease associated with a 23566, 33489, or 57779 wherein the method comprises the steps of determining 23566, 33489, or 57779 sequence information associated with the subject, and based on the 23566, 33489, or 57779 sequence information, determining whether the subject has a 23566, 33489, or 57779-associated disease or disorder or a pre-disposition to a 23566, 33489, or 57779-associated disease or disorder, and/or recommending a particular treatment for the disease, disorder or pre-disease condition. In a preferred embodiment, the method further includes the step of receiving information, e.g., phenotypic or genotypic information, associated with the subject and/or acquiring from a network phenotypic information associated with the subject. The information can be stored in a database, e.g., a relational database. In another embodiment, the method further includes accessing the database, e.g., for records relating to other subjects, comparing the 23566, 33489, or 57779 sequence of the subject to the 23566, 33489, or 57779 sequences in the database to thereby determine whether the subject as a 23566, 33489, or 57779-associated disease or disorder, or a pre-disposition for such.

The present invention also provides in a network, a method for determining whether a subject has a 23566, 33489, or 57779 associated disease or disorder or a pre-disposition to a 23566, 33489, or 57779-associated disease or disorder associated with 23566, 33489, or 57779, said method comprising the steps of receiving 23566, 33489, or 57779 sequence information from the subject and/or information related thereto, receiving phenotypic information associated with the subject, acquiring information from the network corresponding to 23566, 33489, or 57779 and/or corresponding to a 23566, 33489, or 57779-associated disease or disorder (e.g., an inflammatory disorder, neurological disorder, cardiovascular disorder (e.g., disorders of the heart and/or blood vessels), blood clotting disorder, or cellular proliferation and/or differentiation disorder), and based on one or more of the phenotypic information, the 23566, 33489, or 57779 information (e.g., sequence information and/or information related thereto), and the acquired information, determining whether the subject has a 23566, 33489, or 57779-associated disease or disorder or a pre-disposition to a 23566, 33489, or 57779-associated disease or disorder. The method may further comprise the step of recommending a particular treatment for the disease, disorder or pre-disease condition.

The present invention also provides a method for determining whether a subject has a 23566, 33489, or 57779-associated disease or disorder or a pre-disposition to a 23566, 33489, or 57779-associated disease or disorder, said method comprising the steps of receiving information related to 23566, 33489, or 57779 (e.g., sequence information and/or information related thereto), receiving phenotypic information associated with the subject, acquiring information from the network related to 23566, 33489, or 57779 and/or related to a 23566, 33489, or 57779-associated disease or disorder, and based on one or more of the phenotypic information, the 23566, 33489, or 57779 information, and the acquired information, determining whether the subject has a 23566, 33489, or 57779-associated disease or disorder or a pre-disposition to a 23566, 33489, or 57779-associated disease or disorder. The method may further comprise the step of recommending a particular treatment for the disease, disorder or pre-disease condition.

This invention is further illustrated by the following examples that should not be construed as limiting. The contents of all references, patents and published patent applications cited throughout this application are incorporated herein by reference.

### EXAMPLES

### EXAMPLES FOR 47476, 67210, 49875, 46842, 33201, 83378, 84233, 64708, 85041, AND 84234

### Example 1

### Identification and Characterization of Human 47476, 67210, 49875, 46842, 33201, 83378, 84233, 64708, 85041, and 84234 cDNAs

The human 47476 nucleic acid sequence is recited as follows:

(SEQ ID NO:1)

CACCGAGGAGGCCCCAGCTCCCTAGGGGCTGAGAAGCTGGAGTCCTGGGC
AAGGGGAGGAGCTGAGCCCTACTCTTGCAAGACCCCCGGCCTCCTCACCC
CACGCGGGAAGCATGAACAGAAAAGACAGTAAGAGGAAGTCCCACCAGGA
ATGCACCGGAAAAATAGGAGGGCGAGGCCGGCCCCGCCAAGTGCGCCGCC
ACAAGACATGCCCCAGCCCTCGGGAAATCAGCAAGGTCATGGCTTCCATG
AACCTGGGCCTGCTGAGTGAGGGCGGCTGCAGCGAAGATGAGCTGCTGGA
GAAATGCATCCAGTCCTTCGATTCAGCTGGCAGCCTGTGCCACGAGGACC
ACATGCTCAACATGGTGCTGGCCATGCACAGCTGGGTGCTGCCGTCCGCC
GACCTGGCTGCCCGCCTGCTGACCTCATACCAGAAGGCCACAGGGGACAC
CCAGGAGCTGAGACGGCTGCAGATCTGTCACCTGGTCAGGTACTGGCTGA
TGCGACACCCTGAGGTGATGCACCAGGATCCCCAGCTAGAAGAAGTCATA
GGTCGTTTCTGGGCCACCGTGGCCCGGGAGGGCAACTCAGCCCAGAGAAG
ACTGGGAGACTCTTCTGACCTCCTGAGCCCTGGTGGCCCTGGCCCCCCAC
TCCCAATGAGCAGCCCAGGCCTGGGCAAAAAGCGCAAAGTGTCCTTGCTT
TTCGACCACTTGGAGACGGGGGAGCTGGCTCAGCACCTCACCTACCTGGA
GTTCCGGTCCTTCCAGGCTATCACGCCCCAGGACCTGCGGAGCTACGTTT
TGCAGGGCTCAGTACGAGGCTGCCCGGCCCTGGAGGGCTCCGTAGGTCTC
AGCAACAGCGTGTCCCGCTGGGTGCAGGTGATGGTGCTGAGCCGTCCCGG
GCCCCTACAGCGTGCACAGGTGCTGGACAAGTTCATTCACGTGGCACAGA
GGCTCCACCAGCTGCAGAATTTCAACACGCTGATGGCAGTCACAGGGGGC
CTGTGTCACAGTGCCATCTCCAGACTCAAGGACTCCCATGCCCACCTGAG
CCCTGACAGCACCAAGGCCCTCCTGGAGCTCACTGAGCTCCTTGCCTCCC
ACAACAACTACGCCCGCTACCGCCGCACCTGGGCTGGCTGCGCGGGTTTC
CGGCTGCCTGTACTGGGCGTGCACCTCAAGGACCTGGTGTCCCTGCATGA
GGCACAGCCCGACAGGTTGCCTGACGGCCGCCTGCACCTACCCAAGCTGA
ACAACCTCTACCTGCGGCTGCAGGAGCTGGTGGCCCTCCAAGGGCAGCAT
CCACCCTGCAGCGCCAATGAGGATCTGCTGCACCTGCTCACGCTCTCCCT
GGACCTCTTCTACACGGAAGACGAGATCTATGAGCTTTCTTATGCCCGGG
AGCCGCGTTGTCCCAAGAGCCTGCCACCCTCCCCCTTCAATGCACCTCTG
GTGGTGGAGTGGGCCCCTGGTGTGACACCCAAGCCGGACAGGGTCACACT
GGGTCGGCATGTGGAGCAGCTGGTGGAGTCTGTGTTCAAGAATTATGACC
CTGAAGGCCGAGGAACAATCTCTCAGGAGGACTTTGAGCGACTCTCGGGC
AATTTTCCCTTCGCCTGCCATGGGCTTCACCCACCCCCACGCCAGGGGAG
AGGATCCTTCAGCAGAGAGGAGCTGACAGGGTACCTGCTCCGGGCCAGCG
CCATCTGCTCCAAGTTGGGCCTGGCCTTCCTGCACACCTTCCATGAGGTC
ACCTTCCGAAAGCCTACCTTCTGCGACAGCTGCAGTGGCTTCCTCTGGGG
TGTCACCAAGCAAGGCTACCGCTGTCGGGAGTGCGGGCTGTGTTGCCACA
AACACTGCAGAGACCAGGTGAAGGTAGAATGTAAGAAGAGGCCAGGGGCC
AAGGGCGATGCAGGACCCCCCGGAGCTCCTGTCCCATCCACACCAGCTCC
CCATGCCAGCTGTGGCTCCGAGGAAAATCACTCCTACACGCTATCCCTGG
AGCCTGAGACTGGGTGCCAGCTTCGCCATGCCTGGACCCAGACTGAATCC
CCACACCCTTCCTGGGAAACAGATACGGTCCCCTGCCCGGTGATGGACCC
ACCATCAACTGCATCCTCCAAGCTGGATTCCTAGACATCTCTTGGTCTCC
TCTCTTCCTCACTCCCTTCCCCCAGTCAGTCCTGAGTCCTGCCGTCAGAC
TCTGGCAGGGCTCCCAGAGGTAGGCCTCCAGAGGCATCCGCCTTCCCATC
CACACTGCCTTTAGTGGTACGTCCGTCATCTTTTTCCCTGGAAGTGACTT
TCCTCTTTTGCATCCTGGTGGATGCTAATCCTGCTCCCTTTCCCTGCACT
TAGTATCTGTGCGGCTGTGTGTTGGATACATTTAGACTCAGGCCTCATTC
TACCAACTCCCTACATCTCCATTTCTTTTTTTTTTTGAGACAGAGTCTTG
CTCTGTCACCCAGGCTGGAGTGCAATGATGCAATCTCAGCTCACTGCAAC
TTCCGCCTCCTGGGTTCAAGTGATTCTTCTGCCTCAGCCTCCCAAGTAGC
TGGGATTACAGGCACGCGCCACCATGCCCGGCTAATTTTTGTATTTTTAG
TAGAGACGGGGTTTCCCCATGTTGGCCAAGATGGTCTCAAACTCCTGACC
TCATGATCCACCAGCCTCAGCCTCCCAAAGTGCTGGGAATACAGGCGTGA
GCCACCACGCCCAGCTCCTGTTTTTCTGCATAGAATGTGCTCCCTCTGAC
AGCCTATACTTTTTCCCTCTTTATCTCGTTGAACGTCTGTCTTTCCTCAA
CAGGAGCATCAACTCCAGGAGAACAGGGATTTCTATCAATGTCATTCACT
GCTGCATCCCCAGTGCCTAGAACAGGGCTGGCAGGTGGTAGGCGCCTGAC
AGATGCTTGTCTGATGCCTGAATGTCTCCTTACCCATGCCCACGGCACAG
GATAGATGTGCTATAGGGCAAAGAACTTTGAGGGTCGAGCAGCAGGGGCC
CATTCAGTCCCAGGGAGCAGAGACCCCCCCAACCCCTTCACAAACCCCAA
CACCCCTGACTTGGCCCCCACAGAGAGAGGTCCTACAGCTGTCATAAATT
AAATTTATTCTCTGGAAAAAAAAAAAAAAAAAAA.

The human 47476 sequence (SEQ ID NO:1), which is approximately 3134 nucleotides long, includes an initiation codon (ATG) and a termination codon (TAA) which are underscored above. The region between and inclusive of the initiation codon and the termination codon is a methionine-initiated coding sequence of about 2022 nucleotides, including the termination codon (nucleotides indicated as “coding” of SEQ ID NO:1; SEQ ID NO:3). The coding sequence encodes a 673 amino acid protein (SEQ ID NO:2), which is recited as follows:

MNRKDSKRKSHQECTGKIGGRGRPRQVRRHKTCPSPREISKVMASMNLGLLSEGGCSE DELLEKClQSFDSAGSLCHEDHMLNMVLAMHSWVLPSADLAARLLTSYQKATGDTQE LRRLQICHLVRYWLMRHPEVMHQDPQLEEVIGRFWATVAREGNSAQRRLGDSSDLLS PGGPGPPLPMS SPGLGKKRKVSLLFDHLETGELAQHLTYLEFRSFQAITPQDLRSYVLQ GSVRGCPALEGSVGLSNSVSRWVQVMVLSRPGPLQRAQVLDKFIHVAQRLHQLQNFN TLMAVTGGLCHSAISRLKDSHAHLSPDSTKALLELTELLASHNNYARYRRTWAGCAGF RLPVLGVHLKDLVSLHEAQPDRLPDGRLHLPKLNNLYLRLQELVALQGQHPPCSANED LLHLLTLSLDLFYTEDEIYELSYAREPRCPKSLPPSPFNAPLVVEWAPGVTPKPDRVTLG RHVEQLVESVFKNYDPEGRGTISQEDFERLSGNFPFACHGLHPPPRQGRGSFSREELTG YLLRASAICSKLGLAFLHTFHEVTFRKPTFCDSCSGFLWGVTKQGYRCRECGLCCHKH CRDQVKVECKKRPGAKGDAGPPGAPVPSTPAPHASCGSEENHSYTLSLEPETGCQLRH AWTQTESPHPSWETDTVPCPVMDPPSTASSKLDS (SEQ ID NO:2).

The human 67210 nucleic acid sequence is recited as follows:

(SEQ ID NO:4)

GAATTTGTAATACGACTCACTATAGGGAGTCGACCCACGCGTCCGGCGGG
TCGGGGAGGAATATTCTTTTGGAAACGTAATATTGGCCTTGGGGCTCTCC
AGCCCTTTGGGACTTCCAATGGGATCTTAGAAGCAGCCGAAGCAGCGTGA
GGGCGGCAGCCCAGGGCCAGCCACGATTTGAACGCTCTGCCTTGCAGCTC
TTCTGGACCGAGGAGCCCAAAGCCCTACCCTCACCATTCACCAGGTTACA
GTTCTTATCCGCGTGAATACACATGGCTCTGTTACGAAAAATTAATCAGG
TGCTGCTGTTCCTTCTGATCGTGACCCTCTGTGTGATTCTGTATAAGAAA
GTTCATAAGGGGACTGTGCCCAAGAATGACGCAGATGATGAATCCGAGAC
TCCTGAAGAACTGGAAGAAGAGATTCCTGTGGTGATTTGTGCTGCAGCAG
GGAGGATGGGTGCCACTATGGCTGCCATCAATAGCATCTACAGCAACACT
GACGCCAACATCTTGTTCTATGTAGTGGGACTCCGGAATACTCTGACTCG
AATACGAAAATGGATTGAACATTCCAAACTGAGAGAAATAAACTTTAAAA
TCGTGGAATTCAACCCGATGGTCCTCAAAGGGAAGATCAGACCAGACTCA
TCGAGGCCTGAATTGCTCCAGCCTCTGAACTTTGTTCGATTTTATCTCCC
TCTACTTATCCACCAACACGAGAAAGTCATCTATTTGGACGATGATGTAA
TTGTACAAGGTGATATCCAAGAACTGTATGACACCACCTTGGCCCTGGGC
CACGCGGCGGCTTTCTCAGATGACTGCGATTTGCCCTCTGCTCAGGACAT
AAACAGACTCGTGGGACTTCAGAACACATATATGGGCTATCTGGACTACC
GGAAGAAGGCCATCAAGGACCTTGGCATCAGCCCCAGCACCTGCTCTTTC
AATCCTGGTGTGATTGTTGCCAACATGACAGAATGGAAGCACCAGCGCAT
CACCAAGCAATTGGAGAAATGGATGCAAAAGAATGTGGAGGAAAACCTCT
ATAGCAGCTCCCTGGGAGGAGGGGTGGCCACCTCCCCAATGCTGATTGTG
TTTCATGGGAAATATTCCACAATTAACCCCCTGTGGCACATAAGGCACCT
GGGCTGGAATCCAGATGCCAGATATTCGGAGCATTTTCTGCAGGAAGCTA
NATTACTCCACTGGAATGGAAGACATAAACCTTGGGACTTCCCTAGTGTT
CACAACGACTTATGGGAAAGCTGGTTTGTTCCTGACCCTGCAGGGATATT
TAAACTCAATCACCATAGCTGATATAACTCTACCCTTAAAATATTCCCTG
TATAGAAATGTGGAATTGTCCCTTTGTAGCCAACTATAACATTGTTCTTT
ATGAATATTACCTTTGATACATATGATCCACAATATAAAAACCAAAAACT
ACTGTGTGCAAATTATACCTTGGACCATATAGGCATTGATTAACTTCTTT
AAGTACATGTGATAACTATGGAAATCAAGATTATGTGACTGAAAAACATA
AAGGAAGAGACCCATCTAGATAACAGCAATCAACCTGCTTAATTCTGAAT
GACAATTATATCCACAAATTTTTAAAACTTCTACATGTATTTTTCACATG
AAGATCTCCTTAACAGGTTGCCAACCTTTTCTTTTATAAAACTATTACAT
TTAAAATATGGACGTCTGAAAAATAAAATATTCATCATTTTTAAAAAAAA
AAAAAAAAMAAAAAANAAAAAAAAAAAA.

The human 67210 sequence (SEQ ID NO:4), which is approximately 1778 nucleotides long. The nucleic acid sequence includes an initiation codon (ATG) and a termination codon (TGA) which are underscored above. The region between and inclusive of the initiation codon and the termination codon is a methionine-initiated coding sequence of about 1050 nucleotides, including the termination codon (nucleotides indicated as “coding” of SEQ ID NO:4; SEQ ID NO:6). The coding sequence encodes a 349 amino acid protein (SEQ ID NO:5), which is recited as follows:

(SEQ ID NO:5)

MALLRKINQVLLFLLIVTLCVILYKKVHKGTVPKNDADDESETPEELEE
EIPVVICAAAGRMGATMAAINSIYSNTDANILFYVVGLRNTLTRIRKWI
EHSKLREINFKTVEFNIPMVLKGKIRPDSSRPELLQPLNFVRFYLPLLI
HQHEKVIYLDDDVIVQGDIQELYDTTLALGHAAAFSDDCDLPSAQDINR
LVGLQNTYMGYLDYRKKAIKDLGISPSTCSFNPGVIVANMTEWKHQRIT
KQLEKWMQKNVEENLYSSSLGGGVATSPMLIVFHGKYSTINPLWHIRHL
GWNPDARYSEHFLQEAKLLHWNGRHKPWDFPSVHNDLWESWFVPDPAGI
FKLNHHS.

The human 49875 nucleic acid sequence is recited as follows:

(SEQ ID NO:7)

GGCGCGTTCGAGCAGCGGCGACCGACGCGGCGAAGGAGCGCGCCATGGAG
CATGTGACAGAGGGCTCCTGGGAGTCGCTGCCTGTGCCGCTGCACCCGCA
GGTGCTGGGCGCGCTGCGGGAGCTGGGCTTCCCGTACATGACGCCGGTGC
AGTCCGCAACCATCCCTCTGTTCATGCGAAACAAAGATGTCGCTGCAGAA
GCGGTCACAGGTAGTGGCAAAACACTCGCTTTTGTCATCCCCATCCTGGA
AATTCTTCTGAGAAGAGAAGAGAAGTTAAAAAAGAGTCAGGTTGGAGCCA
TAATCATCACCCCCACTCGAGAGCTGGCCATTCAAATAGACGAGGTCCTG
TCGCATTTCACGAAGCACTTCCCCGAGTTCAGCCAGATTCTTTGGATCGG
AGGCAGGAATCCTGGAGAAGATGTTGAGAGGTTTAAGCAACAAGGTGGGA
ACATCATTGTGGCCACTCCAGGCCGCTTGGAGGACATGTTCCGGAGGAAG
GCCGAAGGCTTGGATCTGGCCAGCTGTGTGCGATCCCTGGATGTCCTGGT
GTTGGATGAGGCAGACAGACTTCTGGACATGGGGTTTGAGGCAAGCATCA
ACACCATTCTGGAGTTTTTGCCAAAGCAGAGGAGAACAGGCCTTTTCTCT
GCCACTCAGACGCAGGAAGTGGAGAACCTGGTGAGAGCGGGCCTCCGGAA
CCCTGTCCGGGTCTCAGTGAAGGAGAAGGGCGTGGCAGCCAGCAGTGCCC
AGAAGACCCCCTCCCGCCTGGAAAACTACTACATGGTATGCAAGGCAGAT
GAGAAATTTAATCAGCTGGTCCATTTTCTTCGCAATCATAAGCAGGAGAA
ACACCTGGTCTTCTTCAGCACCTGTGCCTGTGTGGAATACTATGGGAAGG
CTCTGGAAGTGCTGGTGAAGGGCGTGAAGATTATGTGCATTCACGGAAAG
ATGAAATATAAACGCAATAAGATCTTCATGGAGTTCCGCAAATTGCAAAG
TGGGATTTTAGTGTGCACTGATGTGATGGCCCGGGGAATTGATATTCCTG
AAGTCAACTGGGTTTTGCAGTATGACCCTCCCAGCAATGCAAGTGCCTTC
GTGCATCGCTGCGGTCGCACAGCTCGCATTGGCCACGGGGGCAGCGCTCT
GGTGTTCCTCCTGCCCATGGAAGAGTCATACATCAATTTCCTTGCAATTA
ACCAAAAATGCCCCCTGCAGGAGATGAAGCCCCAGAGAAACACAGCGGAC
CTTCTGCCAAAACTCAAGTCCATGGCCCTGGCTGACAGAGCTGTGTTTGA
AAAGGGCATGAAAGCTTTTGTGTCATATGTCCAAGCTTATGCAAAGCATG
AATGCAACCTGATTTTCAGATTAAAGGATCTTGATTTTGCCAGCCTTGCT
CGAGGTTTTGCCCTGCTGAGGATGCCCAAGATGCCAGAATTGAGAGGAAA
GCAGTTTCCAGATTTTGTGCCCGTGGACGTTAATACCGACACGATTCCAT
TTAAAGATAAAATCAGAGAAAAGCAGAGGCAGAAACTCCTGGAGCAACAA
AGAAGAGAGAAAACAGAAAATGAAGGGAGAAGAAAATTCATAAAAAATAA
AGCTTGGTCAAAGCAGAAGGCCAAAAAAGAAAAGAAGAAAAAAATGAATG
AGAAAAGGAAAAGGGAAGAGGGTTCTGATATTGAAGATGAGGACATGGAA
GAACTTCTTAATGACACAAGACTCTTGAAAAAACTTAAGAAAGGCAAAAT
AACTGAAGAAGAATTTGAGAAGGGCTTGTTGACAACTGGCAAAAGAACAA
TCAAGACAGTGGATTTAGGGATCTCAGATTTGGAAGATGACTGCTGATTC
CAGTGCCACAGATGAACCCACAAGGACATAGCTGTTCCCTAACTTGGTGG
ATGGCTCCAGTTTGCTTTTAACGAAAATCACAACTTCAGGAGACATCTGA
AAAGAATGATGTCTCTGAAAGCTGTCCTTTCAGATGAGGGAGAAATGAAG
GATTTCACACTTCAGAATATTTTACTAAAAACATTCCAGTCTTGGCCGGG
TGCGGTGGCTCCTGCCTATAATCCCAGCACTTTGGGAGGCTGAGGCAGGA
GGATCACTTGAGCCCAGGAGTTCAAGACCAGCCTGGGAACACAGCGAGAC
CCTCTCATTAAAAACAACAAAACAAAACAATTCCAGTCTTGGAGTAGTCT
AACAGAAGAAAATGTAAAATTATTTGAGTGTAAATAATAGATGTCAGTAT
TTATCATGATGGGTCACATATAGACATATGTHCATATTATATATATATAT
ATATATATATATATATATATATATATATATATATATATAAGCTCTTTTTT
CTGAGGCTATTTTATAGTTATTTTTAAACATAAAGAYACASAAGTCTTCT
TSACTTCTGATTTTCACAAACCATTCCTCAGTATCTTCAGGCATTTGWCC
TCCTGAATGTGCTTGGCCMTGGGCTTCAGTTATCCTTTGATGTCCTGCAG
GGGTGGCTAATGTGCTGGGGTTTTTCTGTGTTAATAGTCMCAGTATTGTT
TTATTGGTSAATAGCTGAAMAWCAGAGGGATTAAGTCATATTCCGGGAAA
GAGAATTATAGTTTTTATGCCTCCTGTTGAATAAATGGTGTCMTGATTGC
CTGGGTAAAAAAAAAAAAAAWAAAGGKCGKCCGYTAGACTATTTAGAGAA
AAAA.

The human 49875 sequence (SEQ ID NO:7), which is approximately 2704 nucleotides long. The nucleic acid sequence includes an initiation codon (ATG) and a termination codon (TGA) which are underscored above. The region between and inclusive of the initiation codon and the termination codon is a methionine-initiated coding sequence of about 1803 nucleotides, including the termination codon (nucleotides indicated as “coding” of SEQ ID NO:7; SEQ ID NO:9). The coding sequence encodes a 600 amino acid protein (SEQ ID NO:8), which is recited as follows:

(SEQ ID NO:8)

MEHVTEGSWESLPVPLHPQVLGALRELGFPYMTPVQSATIPLFMRNKDVA
AEAVTGSGKTLAFVIPILEILLRREEKLKKSQVGAIIITPTRELAIQIDE
VLSHFTKHFPEFSQILWIGGRNPGEDVERFKQQGGNIIVATPGRLEDMFR
RKAEGLDLASCVRSLDVLVLDEADRLLDMGFEASINTILEFLPKQRRTGL
FSATQTQEVENLVRAGLRNPVRVSVKGVAASSAQKTPSRLENYYMVCKAD
EKFNQLVHFLRNHKQEKHLVFFSTCACVEYYGKALEVLVKGVKIMCIHGK
MKYKRNKIFMEFRKLQSGILVCTDVMARGIDIPEVNWVLQYDPPSNASAF
VHRCGRTARIGHGGSALVFLLPMEESYINFLAINQKCPLQEMKPQRNTAD
LLPKLKSMALADRAVFEKGMKAFVSYVQAYAKHECNLIFRLKDLDFASLA
RGFALLRMPKMPELRGKQFPDFVPVDVNTDTIPFKDKIREKQRQKLLEQQ
RREKTENEGRRKFIKNKAWSKQKAKKEKKKKMNEKRKREEGSDIEDEDME
ELLNDTRLLKKLKKGKITEEEFEKGLLTTGKRTIKTVDLGISDLEDDC.

The human 46842 nucleic acid sequence is recited as follows:

(SEQ ID NO:10)

CGGTCGGGGCGTTACGGCTGCCGCTCGGCGCCGCGGTCTCGTGCCAGTGA
GCGCCGGGCGCCGCAGCCATGACCGTGGAGTTCGAGGAGTGCGTCAAGGA
CTCCCCGCGCTTCAGGGCGACCATTGACGAGGTGGAGACGGACGTGGTGG
AGATTGAGGCCAAACTGGACAAGCTGGTGAAGCTGTGCAGTGGCATGGTG
GAAGCCGGTAAGGCCTACGTCAGCACCAGCAGGCTTTTCGTGAGCGGCGT
CCGCGACCTGTCCCAGCAGTGCCAGGGCGACACCGTCATCTCGGAATGTC
TGCAGAGGTTCGCTGACAGCCTACAGGAGGTGGTGAACTACCACATGATC
CTGTTTGACCAGGCCCAGAGGTCCGTGCGGCAGCAGCTCCAGAGCTTTGT
CAAAGAGGATGTGCGGAAGTTCAAGGAGACAAAGAAGCAGTTTGACAAGG
TGCGGGAGGACCTGGAGCTGTCCCTGGTGAGGAACGCCCAGGCCCCGAGG
CACCGGCCCCACGAGGTGGAGGAAGCCACCGGGGCCCTCACCCTCACCAG
GAAGTGCTTCCGCCACCTGGCACTGGACTATGTGCTCCAGATCAATGTTC
TGCAGGCCAAGAAGAAGTTTGAGATCCTGGACTCTATGCTGTCCTTCATG
CACGCCCAGTCCAGCTTCTTCCAGCAGGGCTACAGCCTCCTGCACCAGCT
GGACCCCTACATGAAGAAGCTGGCAGCCGAGCTGGACCAGCTGGTGATCG
ACTCTGCGGTGGAAAAGCGTGAGATGGAGCGAAAGCACGCCGCCATCCAG
CAGCGGACGCTGCTGCAGGACTTCTCCTACGATGAGTCCAAAGTGGAGTT
TGACGTGGACGCGCCCAGTGGGGTGGTGATGGAGGGCTACCTCTTCAAGA
GGGCCAGCAACGCTTTCAAGACATGGAACCGGCGCTGGTTCTCCATTCAG
AACAGCCAGCTGGTCTACCAGAAGAAGCTCAAGGATGCCCTCACCGTGGT
GGTGGATGACCTCCGCCTGTGCTCTGTGAAGCCGTGTGAGGACATCGAGC
GGAGGTTCTGCTTCGAGGTGCTGTCACCCACCAAGAGCTGCATGCTGCAG
GCTGACTCCGAGAAGCTGCGGCAAGCCTGGGTCCAGGCTGTGCAGGCCAG
CATCGCCTCCGCCTACCGCGAGAGCCCTGACAGTTGCTATAGCGAGAGGC
TGGACCGCACAGCATCCCCGTCCACGAGCAGCATCGACTCCGCCACCGAC
ACTCGGGAGCGTGGCGTGAAGGGCGAGAGTGTGCTGCAGCGTGTGCAGAG
TGTGGCCGGCAACAGCCAGTGCGGCGACTGCGGCCAGCCGGACCCCCGCT
GGGCCAGCATCAACCTGGGCGTGCTGCTCTGCATTGAGTGCTCCGGCATC
CACAGGAGCCTGGGTGTCCACTGCTCCAAGGTGCGGTCCCTGACGCTGGA
CTCGTGGGAGCCTGAGCTGCTAAAGCTGATGTGTGAGCTTGGAAACAGCG
CTGTGAATCAGATCTATGAGGCCCAGTGTGAGGGTGCAGGCAGCAGGAAA
CCCACAGCCAGCAGCTCCCGGCAGGACAAGGAGGCCTGGATCAAGGACAA
ATACGTGGAAAAGAAGTTTCTGCGGAAGGCGCCCATGGCACCAGCCCTGG
AGGCCCCAAGACGCTGGAGGGTGCAGAAGTGCCTGCGGCCCCACAGCTCT
CCCCGCGCTCCCACTGCCCGCCGCAAGGTCCGGCTTGAGCCCGTTCTGCC
CTGTGTGGCCGCTCTGTCCTCAGTGGGCACCCTGGATCGTAAGTTCCGCC
GAGACTCCCTCTTCTGTCCCGACGAGCTGGACTCGCTCTTCTCCTACTTC
GACGCAGGGGCCGCAGGGGCTGGCCCTCGCAGTCTGAGTAGCGACAGTGG
CCTTGGGGGCAGCTCGGATGGCAGCTCGGACGTCCTGGCTTTCGGCTCGG
GCTCTGTGGTGGACAGCGTCACTGAGGAGGAGGGTGCAGAGTCGGAGGAG
TCCAGCGGTGAGGCAGACGGGGACACTGAGGCCGAGGCCTGGGGCCTGGC
GGACGTGCGCGAGCTGCACCCGGGGCTCTTGGCGCACCGCGCAGCGCGTG
CCCGCGACCTTCCTGCGCTGGCGGCGGCGCTGGCCCACGGGGCCGAGGTC
AACTGGGCGGACGCGGAGGATGAGGGCAAGACGCCGCTGGTGCAGGCCGT
GCTAGGGGGCTCCTTGATCGTCTGTGAGTTCCTGCTGCAAAACGGAGCGG
ACGTGAACCAAAGAGACAGCCGGGGCCGGGCGCCCCTGCACCACGCCACG
CTGCTGGGCCGCACCGGCCAGGTTTGCCTGTTCCTGAAGCGGGGCGCGGA
CCAGCACGCCCTGGACCAAGAGCAGCGGGACCCGTTGGCCATCGCAGTGC
AGGCGGCCAACGCTGACATCGTGACACTGCTCCGTCTGGCGCGCATGGCG
GAGGAAATGCGCGAGGCCGAGGCTGCCCCTGGTCCCCCGGGCGCCCTGGC
GGGCAGCCCCACGGAGCTCCAGTTCCGCAGGTGTATCCAGGAGTTCATCA
GCCTCCACCTGGAAGAGAGCTAGGGCCGGGCGGGCCGGGCAGCTGCCACC
CCGCCCGGCCCGACGCCCCGCATGCCCCGAAGTCCCTGGCGCCCACCCGG
CCGCGGCCCTGCGTGTGACCCGCGGGTCGATACCTGGCAGCCCCAGTGCT
GGGGCGCCGCGGCCCTGCTCGCCCAGGAGGAGAGCGA.

The human 46842 sequence (SEQ ID NO:10), which is approximately 2737 nucleotides long. The nucleic acid sequence includes an initiation codon (ATG) and a termination codon (TAG) which are underscored above. The region between and inclusive of the initiation codon and the termination codon is a methionine-initiated coding sequence of about 2505 nucleotides, including the termination codon (nucleotides indicated as “coding” of SEQ ID NO:10; SEQ ID NO:12). The coding sequence encodes a 834 amino acid protein (SEQ ID NO:11), which is recited as follows:

(SEQ ID NO:11)

MTVEFEECVKDSPRFRATIDEVETDVVEIEAKLDKLVKLCSGMVEAGKAY
VSTSRLFVSGVRDLSQQCQGDTVISECLQRFADSLQEVVNYHMILFDQAQ
RSVRQQLQSFVKDVRKFKETKKQFDKVREDLELSLVRNAQAPRHRPHEVE
EATGALTLTRKCFRHLALDYVLQINVLQAKKKFEILDSMLSFMHAQSSFF
QQGYSLLHQLDPYMKKLAAELDQLVIDSAVEKREMERKHAAIQQRTLLQD
FSYDESKVEFDVDAPSGVVMEGYLFKRASNAFKTWNRRWFSIQNSQLVYQ
KKLKDALTVVVDDLRLCSVKPCEDIERRFCFEVLSPTKSCMLQADSEKLR
QAWVQAVQASIASAYRESPDSCYSERLDRTASPSTSSIDSATDTRERGVK
GESVLQRVQSVAGNSQCGDCGQPDPRWASINLGVLLCIECSGIHRSLGVH
CSKVRSLTLDSWEPELLKLMCELGNSAVNQIYEAQCEGAGSRKPTASSSR
QDKEAWIKDKYVEKKFLRKAPMAPALEAPRRWRVQKCLRPHSSPRAPTAR
RKVRLEPVLPCVAALSSVGTLDRKFRRDSLFCPDELDSLFSYFDAGAAGA
GPRSLSSDSGLGGSSDGSSDVLAFGSGSVVDSVTEEEGAESEESSGEADG
DTEAEAWGLADVRELHPGLLAHRAARARDLPALAAALAHGAEVNWADAED
EGKTPLVQAVLGGSLIVCEFLLQNGADVNQRDSRGRAPLHHATLLGRTGQ
VCLFLKRGADQHALDQEQRDPLAIAVQAANADIVTLLRLARMAEEMREAE
AAPGPPGALAGSPTELQFRRCIQEFISLHLEES.

The human 33201 nucleic acid sequence is recited as follows:

(SEQ ID NO:13)

CACGCGTCCGGGCCTGGGGGCGAGCTGGGGTCGTGCAGTACAGCCTCTTT
CCGGCAAATCACGCGAGATTTCGTTCACCCGGGCTCCACACGGAGTATTT
TATACAGAAATCTTGTGAAACCACTGCCCAACCAGAGCAATGATTGTTCA
AAGAGTGGTATTGAATTCTCGACCTGGAAAAAATGGTAATCCAGTGGCAG
AGAATTTCCGAATGGAAGAAGTCTATTTACCAGATAATATTAATGAAGGA
CAAGTACAAGTTAGAACTCTTTATCTTTCTGTGGATCCTTACATGCGTTG
TAGAATGAATGAAGACACTGGCACTGATTATATAACACCTTGGCAGCTAT
CTCAAGTCGTTGATGGTGGAGGTATTGGAATTATAGAAGAAAGCAAACAC
ACAAATTTGACTAAAGGCGATTTTGTGACTTCTTTCTATTGGCCCTGGCA
AACCAAGGTTATTCTGGATGGAAATAGCCTTGAAAAGGTAGACCCACAAC
TTGTGGATGGACACCTTTCATATTTTCTTGGAGCTATAGGTATGCCTGGT
TTGACTTCCTTGATTGGGATACAGGAAAAAGGTCATATAACTGCTGGATC
TAATAAGACAATGGTTGTCAGTGGGGCCGCAGGTGCCTGTGGATCTGTGG
CTGGGCAGATTGGCCATTTCTTAGGTTGTTCCAGAGTGGTGGGAATTTGT
GGAACACATGAGAAATGCATCCTCTTGACCTCAGAACTGGGCTTTGATGC
TGCAATTAATTATAAAAAGACAATGTGGCAGAACAGCTCCGTGAATCATG
CCCAGCTGGAGTGGATGTTTATTTTGACAATGTTGGTGGTAACATCAGTG
ATACAGTGATAAGTCAGATGAATGAGAACAGCCACATCATCCTGTGTGGT
CAAATTTCTCAGTACAACAAAGATGTGCCTTATCCTCCCCCGCTATCCCC
TGCTATAGAGGCAATCCAGAAAGAAAGAAACATCACAAGGGAAAGATTTC
TGGTATTAAATTATAAAGACAAATTTGAGCCTGGCATTCTACAGCTGAGT
CAGTGGTTTAAAGAAGGAAAGCTAAAGATTAAAGAGACGGTAATAAATGG
GTTGGAAAACATGGGAGCTGCATTCCAGTCCATGATGACAGGAGGTAACA
TTGGAAAGCAGATAGTTTGCATTTCAGAAGAAATCTCTTTGTAATTGCTG
TAAATGTCATCAAGGCAATCATAGATTTCTTTTCCATTTTGCATATTTTC
AAAGATATGTTAAAAAATCCTTAGACTATACATAGCTCTTGATTTAAATG
TGATCATAGGTGTTATTTTTAGTTGCATAGGGTATTTGATACAATCATTA
ATGGATCATACACAATAGGTTTTTAAAAATTAATAACTTTTAGTAATTAC
TTTTATTAATTTAAAATAGAACACTTGAGAGGCACTTTGTAAAGATTTGT
TAAACTGGAAACGTTTTACATGATCTGATACAACCATTAATGAATCATAC
ACAATAGGTTTTTTAAAATTAATATTAATAACTTTTATTAATTTAAAATA
GAATGCTTAAAATAAAATAGAATGCTTGAGAGGCACTGAGTAAAGATTTG
TTGAACTGGAAATGTTTTACATGATTTTTAAACTGAAACTTGGTGTAAAA
ATAGAATTGAGATGGCCTTTTTTTCACATTGTAGACTGAAAAGAGACTTA
ATGGTATGATGTGTACC.

The human 33201 sequence (SEQ ID NO:13), which is approximately 1718 nucleotides long. The nucleic acid sequence includes an initiation codon (ATG) and a termination codon (TAA) which are underscored above. The region between and inclusive of the initiation codon and the termination codon is a methionine-initiated coding sequence of about 1056 nucleotides, including the termination codon (nucleotides indicated as “coding” of SEQ ID NO:13; SEQ ID NO:15). The coding sequence encodes a 351 amino acid protein (SEQ ID NO:14), which is recited as follows:

(SEQ ID NO:14)

MIVQRVVLNSRPGKNGNPVAENFRMEEVYLPDNINEGQVQVRTLYLSVDP
YMRCRMNEDTGTDYITPWQLSQVVDGGGIGIIEESKHTNLTKGDFVTSFY
WPWQTKVILDGNSLEKVDPQLVDGHLSYFLGAIGMPGLTSLIGIQEKGHI
TAGSNKTMVVSGAAGACGSVAGQIGHFLGCSRVVGICGTHEKCILLTSEL
GFDAAINYKKDNVAEQLRESCPAGVDVYFDNVGGNISDTVISQMNENSHI
ILCGQISQYNKDVPYPPPLSPAIEAIQKERNITRERFLVLNYKDKFEPGI
LQLSQWFKEGKLKIKETVINGLENMGAAFQSMMTGGNIGKQIVCISEEJS
L.

The human 83378 nucleic acid sequence is recited as follows:

(SEQ ID NO:16)

GCCCTTATGGGCCGCTACTCTGGCAAGACGTGCCGGCTGCTCTTCATGCT
GGTGCTCACCGTCGCCTTCTTCGTGGCGGAGCTGGTCTCCGGCTACCTGG
GCAACTCCATCGCGCTGCTCTCCGACTCCTTCAACATGCTCTCCGACCTG
ATCTCGCTGTGCGTGGGCCTGAGCGCCGGCTACATCGCCCGGCGCCCCAC
CCGGGGCTTCAGCGCCACCTACGGCTACGCCCGCGCCGAGGTGGTGGGCG
CGCTGAGCAACGCGGTCTTCCTCACCGCGCTCTGCTTCACCATCTTCGTG
GAGGCCGTGCTGCGCCTGGCCCGGCCCGAGCGCATCGATGACCCCGAGCT
GGTGCTCATCGCCGGCGTCCTGGGGCTGTTGGTCAACGTGGTGGGGCTGC
TCATCTTCCAGGACTGCGCCGCCTGGTTCGCGTGCTGCCTCCGGGGACGC
AGTCGCCGCCTGCAGCAGCGGCAGCAGCTGGCGGAGGGCTGTGTCCCCGG
CGCTTTCGGGGGGCCTCAGGGCGCGGAGGACCCGCGGCGCGCGGCGGACC
CGACAGCCCCAGGCTCGGACTCGGCCGTAACCCTCCGGGGGACCTCGGTG
GAAAGGAAGCGGGAGAAGGGGGCGACCGTGTTCGCAAACGTAGCAGGTGA
TTCCTTCAACACCCAGAATGAGCCAGAAGACATGATGAAAAAAGAGAAAA
AGTCTGAAGCTCTGAATATCAGAGGTGTACTTTTGCATGTGATGGGAGAT
GCCCTGGGGTCCGTGGTTGTGGTCATCACGGCCATCATATTCTATGTGCT
TCCCCTGAAGAGTGAGGACCCGTGTAACTGGCAGTGTTACATTGACCCCA
GCCTGACTGTCCTCATGGTCATCATCATTTTGTCATCTGCCTTCCCGCTT
ATCAAGGAGACCGCTGCCATTCTGCTACAGATGGTCCCAAGGAGTCAACA
TGGAAGAGCTGATGAGTAAACTCTCTGCTGTGCCTGGAATTAGCAGTGTA
CATGAAGTGCACATCTGGGAACTTGTAAGTGGAAAGATTATTGCCACCCT
GCACATCAAGTATCCTAAGGACAGGGGATATCAAGATGCCAGCACAAAAA
TTCGAGAAATCTTCCACCATGCGGGAATCCACAATGTGACCATCCAGTTT
GAAAATGTGGACTTGAAGGAACCCCTGGAGCAGAAGGACTTACTGTTGCT
CTGCAACTCACCCTGCATCTCCAAGGGCTGTGCTAAGCAGCTGTGTTGTC
CCCCCGGGGCACTGCCTCTGGCTCACGTCAATGGCTGTGCTGAGCAAAAT
GGTGGGCCCTCTCTAGACACATACGGAAGTGATGGCCTCAGTAGAAGAGA
CGCAAGAGAAGTGGCTATTGAAGTGTCTTTGGATAGCTGTCTGAGTGACC
ACGGACAAAGTCTTAACAAACTCAGGAGGACCAATGTTATGTCAACAGAA
CGCATTTTTAATCTGGTACTCACATAATCAGACCATATAGACGAGGCACT
TTGGAACCACAAGCTTGGCTCACAAAAAGAGCTTTCTGGGTTGTAGGCCC
AGACTAGACTTGCAGCATGCATGCTCTGTGTTCACTAGGGGTTGGCTGTT
TGGGATTTTAGTTAAACGTGTCTGTGAATTTTTATTGTTAACTAACTCCT
TTCCATTCCCCTGGGTGTCTCATGCTGCTCTTTGACTGTTTCAGCTTGAA
CATGCATTTTCTAAAGCAAACTGCACTAGTGTATATATCAGGGACATTAA
AGTCTGGGACTGGGGCTCAATAAAAAAAAAAAAAAAAAAAAAAAAAAAAA
AAAAAAAAAAAAAAGGGCGGCCGC.

The human 83378 sequence (SEQ ID NO:16) is approximately 1827 nucleotides long. The nucleic acid sequence includes an initiation codon (ATG) and a termination codon (TAA) which are underscored above. The region between and inclusive of the initiation codon and the termination codon is a methionine-initiated coding sequence of about 1458 nucleotides, including the termination codon (nucleotides indicated as “coding” of SEQ ID NO:16; SEQ ID NO:18). The coding sequence encodes a 485 amino acid protein (SEQ ID NO:17), which is recited as follows:

(SEQ ID NO:17)

MGRYSGKTCRLLFMLVLTVAFFVAELVSGYLGNSIALLSDSFNMLSDLIS
LCVGLSAGYIARRPTRGFSATYGYARAEVVGALSNAVFLTALCFTIFVEA
VLRLARPERIDDPELVLIAGVLGLLVNVVGLUFQDCAAWFACCLRGRSRR
LLQQRQQLAEGCVPGAYGGPQGAEDPRRNADPTAPGSDSAVTLRGTSVER
KREKGATVFANVAGDSFNTQNEPEDMMKKEKKSEALNIRGVLLHVMGDAL
GSVVVVITAIIFYVLPLKSEDPCNWQCYIDPSLTVLMVIIILSSAFPLIK
FTAAILLQMVPKGVNMEELMSKLSAVPGISSVHEVHIWELVSGKIIATLH
IKYPKDRGYQDASTKIREIFHHAGIHNVTIQFENVDLKEPLEQKDLLLLC
NSPCISKGCAKQLCCPPGALPLAHVNGCAEQNGGPSLDTYGSDGLSRRDA
REVAIEVSLDSCLSDHGQSLNKTQEDQCYVNRTHF.

The human 84233 nucleic acid sequence is recited as follows:

(SEQ ID NO:19)

CCGGGCAGGTACGCGGGGAAGCTCTTGAGCTCCTCTACCTCTTAGAAAGC
ACAATTGAATCAGATATCATATGAAAGACATACACACTTCATGTAATGCT
ACCTGCGAGTCTCCCTAGAAAAGCAGTTTTTGTAGGTGAAAACAATGAAG
CCAGGTAATATTGCAAGGAGGCTGTAATTTTAGCAGACCTACCAACAACA
CTGATGTAGGAAGCTCATTATTTTAATTTCTGGAGCCTTTTAATTTTTTC
TTTAGAAAGTGTATAAATAATTGCAGTGCTGCTTTGCTTCCAAAACTGGG
CAGTGAGTTCAACAACAACGACAACAACAGCCGCAGCTCATCCTGGCCGT
CATGGAGTTTCTTGAAAGAACGTATCTTGTGAATGATAAAGCTGCCAAGA
TGTATGCTTTCACACTAGAAAGGAGCTGCAAATGAACACTTCATAGCAAT
GTGGAACTCCAACAGAAACCGGTGAATAAAGATCAGTGTCCCAGAGAGAG
ACCAGAGGAGCTGGAGTCAGGAGGCATGTACCACTGCCACAGTGGCTCCA
AGCCCACAGAAAAGGGGGCGAATGAGTACGCCTATGCCAAGTGGAAACTC
TGTTCTGCTTCAGCAATATGCTTCATTTTCATGATTGCAGAGGTCGTGGG
TGGGCACATTGCTGGGAGTCTTGCTGTTGTCACAGATGCTGCCCACCTCT
TAATTGACCTGACCAGTTTCCTGCTCAGTCTCTTCTCCCTGTGGTTGTCA
TCGAAGCCTCCCTCTAAGCGGCTGACATTTGGATGGCACCGAGCAGAGAT
CCTTGGTGCCCTGCTCTCCATCCTGTGCATCTGGGTGGTGACTGGCGTGC
TAGTGTACCTGGCATGTGAGCGCCTGCTGTATCCTGATTACCAGATCCAG
GCGACTGTGATGATCATCGTTTCCAGCTGCGCAGTGGCGGCCAACATTGT
ACTAACTGTGGTTTTGCACCAGAGATGCCTTGGCCACAATCACAAGGAAG
TACAAGCCAATGCCAGCGTCAGAGCTGCTTTTGTGCATGCCCTTGGAGAT
CTATTTCAGAGTATCAGTGTGCTAATTAGTGCACTTATTATCTACTTTAA
GCCAGAGTATAAAATAGCCGACCCAATCTGCACATTCATCTTTTCCATCC
TGGTCTTGGCCAGCACCATCACTATCTTAAAGGACTTCTCCATCTTACTC
ATGGAAGGTGTGCCAAAGAGCCTGAATTACAGTGGTGTGAAAGAGCTTAT
TTTAGCAGTCGACGGGGTGCTGTCTGTGCACAGCCTGCACATCTGGTCTC
TAACAATGAATCAAGTAATTCTCTCAGCTCATGTTGCTACAGCAGCCAGC
CGGGACAGCCAAGTGGTTCGGAGAGAAATTGCTAAAGCCCTTAGCAAAAG
CTTTACGATGCACTCACTCACCATTCAGATGGAATCTCCAGTTGACCAGG
ACCCCGACTGCCTTTTCTGTGAAGACCCCTGTGACTAGCTCAGTCACACC
GTCAGTTTCCCAAATTTGACAGGCCACCTTCAAACATGCTGCTATGCAGT
TTCTGCATCATAGAAAATAAGGAACCAAAGGAAGAAATTCATGTCATGGT
GCAATGCATATTTTATCTATTTATTTAGTTGCATTCACCATGAAGGAAGA
GGCACTGAGATCCATCAATCAATTGGATTATATACTGATCAGTAGCTGTG
TTCAATTGCAGGAATGTGTATATAGATTATTCCTGAGTGGAGCCGAAGTA
ACAGCTGTTTGTAACTATCGGCAATACCAAATTCATCTCCCTTCCAATAA
TGCATCTTGAGAACACATAGGTAAATTTGAACTCAGGAAAGTCTTACTAG
AAATCAGTGGAAGGGACAAATAGTCACAAAATTTTACCAAAACATTAGAA
ACAAAAAATAAGGAGAGCCAAGTCAGGAATAAAAGTGACTCTGTATGCTA
ACGCCACATTAGAACTTGGTTCTCTCACCAAGCTGTAATGTGATTTTTTT
TTCTACTCTGAATTGGAAATATGTATGAATATACAGAGAAGTGCTTACAA
CTAATTTTTATTTACTTGTCACATTTTGGCAATAAATCCCTCTTATTTCT
AAAAAAAAAAAAAAAAAAAAAAAAAAAAAAAAAAAAAAAAAAAAAAAAAA
AAAAAAAAAAAAAAA.

The human 84233 sequence (SEQ ID NO:19) is approximately 2165 nucleotides long. The nucleic acid sequence includes an initiation codon (ATG) and a termination codon (TAG) which are underscored above. The region between and inclusive of the initiation codon and the termination codon is a methionine-initiated coding sequence of about 963 nucleotides, including the termination codon (nucleotides indicated as “coding” of SEQ ID NO:19; SEQ ID NO:21). The coding sequence encodes a 320 amino acid protein (SEQ ID NO:20), which is recited as follows:

(SEQ ID NO:20)

MYHCHSGSKPTEKGANEYAYAKWKICSASAICFIFMIAEVVGGHIAGSLA
VVTDAAHLLIDLTSFLLSLFSLWLSSKPPSKRLTFGWHRAEILGALLSIL
CIWVVTGVLVYLACERLLYPDYQIQATVMIIVSSCAVAANIVLTVVLHQR
CLGHNHKEVQANASVRAAFVHALGDLFQSISVLISALIWFKPEYKIADPI
CTFWSILVLASTITILKDFSILLMEGVPKSLNYSGVKELILAVDGVLSVH
SLHIWSLTMNQVILSAHVATAASRDSQVVRREIAKALSKSFTMHSLTIQM
ESPVDQDPDCLFCEDPCD.

The human 64708 nucleic acid sequence is recited as follows:

(SEQ ID NO:22)

GCACGAGGGCGGGAGCTGTGCAGCTCCTTATCATGGGGACAATTCATCTC
TTTCGAAAACCACAAAGATCCTTTTTTGGCAAGTTGTTACGGGAATTTAG
ACTTGTAGCAGCTGACCGAAGGTCCTGGAAGATACTGCTCTTTGGTGTAA
TAAACTTGATATGTACTGGCTTCCTGCTTATGTGGTGCAGTTCTACTAAT
AGTATAGCTTTAACTGCCTATACTTACCTGACCATTTTTGATCTTTTTAG
TTTAATGACATGTTTAATAAGTTACTGGGTAACATTGAGGAAACCTAGCC
CTGTCTATTCATTTGGGTTTGAAAGATTAGAAGTCCTGGCTGTATTTGCC
TCCACAGTCTTGGCACAGTTGGGAGCTCTCTTTATATTAAAGAAAGTGCA
GAACGCTTTTTGGAACAGCCCGAGATACACACGGGAAGATTATTAGTTGG
TACTTTTGTGGCTCTTTGTTTCAACCTGTTCACGATGCTTTCTATTCGGA
ATAAACCTTTTGCTTATGTCTCAGAAGCTGCTAGTACGAGCTGGCTTCAA
GAGCATGTTGCAGATCTTAGTCGAAGCTTGTGTGGAATTATTCCGGGACT
TAGCAGTATCTTCCTTCCCCGAATGAATCCATTTGTTTTGATTGATCTTG
CTGGAGCATTTGCTCTTTGTATTACATATATGCTCATTGAAATTAATAAT
TATTTTGCCGTAGACACTGCCTCTGCTATAGCTATTGCCTTGATGACATT
TGGCACTATGTATCCCATGAGTGTGTACAGTGGGAAAGTCTTACTCCAGA
CAACACCACCCCATGTTATTGGTCAGTTGGACAAACTCATCAGAGAGGTA
TCTACCTTAGATGGAGTTTTAGAAGTCCGAAATGAACATTTTTGGACCCT
AGGTTTTGGCTCATTGGCTGGATCAGTGCATGTAAGAATTCGACGAGATG
CCAATGAACAAATGGTTCTTGCTCATGTGACCAACAGGCTGTACACTCTA
GTGTCTACTCTAACTGTTCAAATTTTCAAGGATGACTGGATTAGGCCTGC
CTTATTGTCTGGGCCTGTTGCAGCCAATGTCCTAAACTTTTCAGATCATC
ACGTAATCCCAATGCCTCTTTTAAAGGGTACTGATGATTTGAACCCAGTT
ACATCAACTCCAGCTAAACCTAGTAGTCCACCTCCAGAATTTTCATTTAA
CACTCCTGGGAAAAATGTGAACCCAGTTATTCTTCTAAACACACAAACAA
GGCCTTATGGTTTTGGTCTCAATCATGGACACACACCTTACAGCAGCATG
CTTAATCAAGGACTTGGAGTTCCAGGKAKTTGGAGCAACTCAAGGATTGA
GGACTGGTTTTACAAATATACCAAGTAGATATGGAACTAATAATAGAATT
GGACAACCAAGACCATGATAGACTCTAACTTATTTTTATAAGGAATATTG
ACTCCTTGGCTTCCAATTTATTTAGTAATCCAACTTTGCATTGACTGTTT
AATCATTTACTCTAAATGTTAGATAATAGTAGTCTTGTTCACATTTCATG
ALACCTATGAAACTATATTTTTGTAAAATGTATTTGTGACAGTGAAATCC
TCGTAAATGTTAAAGGCTTTAAATAGGCTTCCTTTAGAAAATGTGTTTCT
TTAAATTTGGATTTTGGTATCTTTGGTTTTGTAGTTGACTGCAGTGTGAT
GTGACCTTACCTTTATAAGAGCCACTTGATGGAGTAGATCTGTCACATTA
CTAAGATACGATATTTCTTTTTTTTTCCGAGACGGAGTCTTGCTCTGCCA
CTGTGCCCGGCCAATACATTATTATTAACTTAAGGCTGTACTTTATTAAG
GCTTCCTTAGTTTTTGTTTTGTTTTGTTTTTTGAGATGGAGTCTCACTCT
GTCGCCCAGGCTGGAATGCAGTGGCATGATCTCAGCTCACTGCAACCTCT
GCCTCCTGAGGCAGGAGAGTTGCTTGAACCTGGGGGGCGGAGGTTGCAGT
GAGCCACTGCACTCCAGCCTGATGACAGAGCAAGACTCAGTCTCAAAAAT
AAATAAAAATAATAAAACCTCCATAAGTAATCCTGAAAAAAAAAAAAAAA
AAAAAATCCGAGGGGGGGCGCCGGTACCCA.

The human 64708 sequence (SEQ ID NO:22) is approximately 2130 nucleotides long. The nucleic acid sequence includes an initiation codon (ATG) and a termination codon (TGA) which are underscored above. The region between and inclusive of the initiation codon and the termination codon is a methionine-initiated coding sequence of about 1386 nucleotides, including the termination codon (nucleotides indicated as “coding” of SEQ ID NO:22; SEQ ID NO:24). The coding sequence encodes a 461 amino acid protein (SEQ ID NO:23), which is recited as follows:

(SEQ ID NO:23)

MGTIHLFRKPQRSFFGKLLREFRLVAADRRSWKILLFGVINLICTGFLLM
WCSSTNSIALTAYTYLTIFDLFSLMTCLISYWVTLRKPSPVYSFGFERLE
VLAVFASTVLAQLGALFILKESAERFLEQPEIITTGRLLVGTFVALCFNL
FTMLSIRNKPFAYVSEAASTSWLQEHVALSRSLCGIIPGLSSIFLPRMNP
FVLIDLAGAFALCITYMLIEINNYFAVDTASAIAIALMTFGTMYPMSVYS
GKVLLQTTPPHVIGQLDKLIREVSTLDGVLEVRNEHFWTLGFGSLAGSVH
VRIRRDANEQMVLAHVTNRLYTLVSTLTVQIFKDDWIRPALLSGPVAANV
LNFSDHHVIMPLLKGTDDLNPVTSTPAKPSSPPPEFSFNTPGKNVNPVIL
LNTQTRPYGFGLNHGHTPYSSMLNQGLGVPGIGATQGLRTGFTNIPSRYG
TNNRIGQPRP.

The human 85041 nucleic acid sequence is recited as follows:

(SEQ ID NO:25)

TTGCTGGGCCTGATGACGTGGCTTGGCAACGTCCCTACCGCCGCTGCTTC
CCGGGAACCTGGCGCCGCCGGAACTGATCGCGGCCTAGTCCCGACGCGTG
TGTGCTAGTGAGCCGGAGCCGGCGACGGCGGCAGTGGCGGCCCGGCCTGC
AGGAGCCCGACGGGGTCTCTGCCATGGGGGAGTGACGCGCCTGCACCCGC
TGTTCCGCGGCAGCGGCGAGACATGAGGAGACCCCGCGACAGGGGCAGCG
GCGGCGGCTCGTGAGCCCCGGGATGGAGGAGAAATACGGCGGGGACGTGC
TGGCCGGCCCCGGCGGCGGCGGCGGCCTTGGGCCGGTGGACGTACCCAGC
GCTCGATTAACAAAATATATTGTGTTACTATGTTTCACTAAATTTTTGAA
GGCTGTGGGACTTTTCGAATCATATGATCTCCTAAAAGCTGTTCACATTG
TTCAGTTCATTTTTATATTAAAACTTGGGACTGCATTTTTTATGGTTTTG
TTTCAAAAGCCATTTTCTTCTGGGAAAACTATTACCAAACACCAGTGGAT
CAAAATATTTAAACATGCAGTTGCTGGGTGTATTATTTCACTCTTGTGGT
TTTTTGGCCTCACTCTTTGTGGACCACTAAGGACTTTGCTGCTATTTGAG
CACAGTGATATTGTTGTCATTTCACTACTCAGTGTTTTGTTCACCAGTTC
TTGGAGGAGGACCAGCAAAGACAAGGGGAGCTGCTTTTTTCATTATTGCT
GTGATCTGTTTATTGCTTTTTGACAATGATGATCTCATGGCTAAAATGGC
TGAACACCCTGAAGGACATCATGACAGTGCTCTAACTCATATGCTTTACA
CAGCCATTGCCTTCTTAGGTGTGGCAGATCACAAGGGTGGAGTATTATTG
CTAGTACTGGCTTTGTGTTGTAAAGTTGGTTTTCATACAGCTTCCAGAAA
GCTCTCTGTCGACGTTGGTGGAGCTAAACGTCTTCAAGCTTTATCTCATC
TTGTTTCTGTGCTTCTCTTGTGCCCATGGGTCATTGTTCTTTCTGTGACA
ACTGAGAGTAAAGTGGAGTCTTGGTTTTCTCTCATTATGCCTTTTGCAAC
GGTTATCTTTTTTGTCATGATCCTGGATTTCTACGTGGATTCCATTTGTT
CAGTCAAAATGGAAGTTTCCAAATGTGCTCGTTATGGATCCTTTCCCATT
TTTATTAGTGCTCTCCTTTTTGGAAATTTTTGGACACATCCAATAACAGA
CCAGCTTCGGGCTATGAACAAAGCAGCACACCAGGAGAGCACTGAACACG
TCCTGTCTGGAGGAGTGGTAGTGAGTGCTATATTCTTCATTTTGTCTGCC
AATATCTTATCATCTCCCTCTAAGAGAGGACAAAAAGGTACCCTTATTGG
ATATTCTCCTGAAGGAACACCTCTTTATAACTTCATGGGTGATGCTTTTC
AGCATAGCTCTCAATCGATCCCTAGGTTTATTAAGGAATCACTAAAACAA
ATTCTTGAGGAGAGTGACTCTAGGCAGATCTTTTACTTCTTGTGCTTGAA
TCTGCTTTTTACCTTTGTGGAATTATTCTATGGCGTGCTGACCAATAGTC
TGGGCCTGATCTCGGATGGATTCCACATGCTTTTTGACTGCTCTGCTTTA
GTCATGGGACTTTTTGCTGCCCTGATGAGTAGGTGGAAAGCCACTCGGAT
TTTCTCCTATGGGTACGGCCGAATAGAAATTCTGTCTGGATTTATTAATG
GACTTTTTCTAATAGTAATAGCGTTTTTTGTGTTTATGGAGTCAGTGGCT
ARATTGATTGATCCTCCAGAATTAGACACTCACATGTTAACACCAGTYTC
AGTTGGAGGGCTGATAGTAAACCTTATTGGTATCTGTGCCTTTAGCCATG
CCCATAGCCATGCCCATGGAGCTTCTCAAGGAAGCTGTCACTCATCTGAT
CACAGCCATTCACAYCATATGCATGGACACAGTGACCATGGGCATGGTCA
CAGCCACGGATCTGCGGGTGGAGGCATGAATGCTAACATGAGGGGTGTAT
TTCTACATGTTTTGGCAGATACACTTGGCAGCATTGGTGTGATCGTATCC
ACAGTTCTTATAGAGCAGTTTGGATGGTTCATCGCTGACCCACTCTGTTC
TCTTTTTATTGCTATATTAATATTTCTCAGTGTTGTTCCACTGATTAAAG
ATGCCTGCCAGGTTCTACTCCTGAGATTGCCACCAGAATATGAAAAAGAA
CTACATATTGCTTTAGAAAAGGTACAGGAAATTGAAGGATTAATATCATA
CCGAGACCCTCATTTTTGGCGTCATTYTGCTAGTATTGTGGCAGGAACAA
TTCATATACAGGTGACATYTGATGTGCTAGAACAAAGAATAGTACRGCAG
GTTACAGGAATACTTAAAGATGCTGGAGTAAACAATTTAACAATTCAAGT
GGAAAAGGAGGCATACTTTCAACATATGTCTGGCCTAAGTACTGGATTTC
ATGATGTTCTGGCTATGACAAAACAAATGGAATCCATGAAATACTGCAAA
GATGGTACTTACATCATGTGAGATAACTCAAGRATTACCCCTGGRGPATA
AACAATGAAGRTTAAATGACTCAGTATTTGTAATATTGCCAGAAGGATAA
AAATTACACATTAACTGTACAGAAACAGAGTTCCCTACTACTGGATCAAG
GAATCTTTCTTGAAGGAAATTTAAATACAGAATGAAACATTAATGGTAAA
GTGGAGTAATTATTTAAATTATGTGTATAAAAGGAATCAAATTTTGAGTA
AACATGATGTATTACATCATCTTCAAAAATAGATATGATGGATTCTAGTG
AAGACCAAAATTACTTCTGTTTACTTTCTATCAGGAAGCATCTCCATTGT
AATATGTATTTACATGTTTATTACAAAGACCCAAATGAAAAATTTTTAGT
CCATTTTTTGCATAGCCTAAAGATAAAATAGGAATAAAAGTTCTATATTT
ATGGGATTTTCTGTATATAAAACTGGTTTCTTTCTTATAACTTAAGTCCA
TTAAGTAAAATCTGTATTGCCACTTTAAATGTAAACTAAATTATTTGGGA
GAAACTTCAACCACTGATATGAGATAAGCAATGAGAATAGGGAAGTGTAT
AACATCACAGTTTTTGATGTATTACAAAAATCAACCACTTTATAAAATAA
ATTTTTTTTACTTTTGGTAAAAAAAAAAAAAAAAAAAAAAAAAAAAAAAA
AAAAAAAAAGCGGCCGCTGAATTCTAGNTAGAATTCAGCGGCCGCTGAAT
NCTA.

The human 85041 sequence (SEQ ID NO:25) is approximately 3304 nucleotides long. The nucleic acid sequence includes an initiation codon (ATG) and a termination codon (TGA) which are underscored above. The region between and inclusive of the initiation codon and the termination codon is a methionine-initiated coding sequence of about 2298 nucleotides, including the termination codon (nucleotides indicated as “coding” of SEQ ID NO:25; SEQ ID NO:27). The coding sequence encodes a 765 amino acid protein (SEQ ID NO:26), which is recited as follows:

(SEQ ID NO:26)

MEEKYGGDVALAGPGGGGGLGPVDVPSARLTKYIVLLCFTKFLKAVGLFE
SYDLLKAVHIVQFIFILKLGTAFFMVLFQKPFSSGKTITIKHQWIKIFKH
AVAGCIISLLWFFGLTLCGPLRTLLLFEHSDIVVISLLSVLFTSSGGGPA
KTRGAAYFIIAVICLLLFDNDDLMAKMAEHPEGHHDSALTHMLYTAIAFL
GVADHKGGVLLLVLALCCKVGFHTASRKLSVDVGGAKRLQALSHLVSVLL
LCPWVIVLSVTTESKVESWFSLTMPFATVIFFVMILDFYVDSICSVKMEV
SKCARYGSFPIFISALLFGNFWTHPITDQLRAMNKAAHQESTEHVLSGGV
VVSAIFFILSANILSSPSKRGQKGTLIGYSPEGTPLYNFMGDAYQHSSQS
HPRFIKESLKQILEESDSRQIFYFLCLNLLFTFVELFYGVLTNSLGLISD
GFHMLFDCSALVMGLFAALMSRWKATRIFSYGYGRIEILSGFNGLFLIVI
AFFVFMESVARLIDPPELDTHMLTPVSVGGLIVNLIGICAFSHAHSHAHG
ASQGSCHSSDHSHSHHMHGHSDHGHGHSHGSAGGGMNANMRGVFLHVLAD
TLGSIGVWSTVLIEQFGWFIADPLCSLFIAILTFLSVVPLIKDACQVLLL
RLPPEYEKELHIALEKIQEIEGLISYRDPHFWRHSASIVAGTIHIQVTSD
VLEQRIVQQVTGILKDAGVNNLTIQVEKEAYFQHMSGLSTGFHDVLAMTK
QMESMKYCKDGTYIM.

The human 84234 nucleic acid sequence is recited as follows:

(SEQ ID NO:28)

GTCGACCCACGCGTCCGGTCTGTGTCTGTCTGTGTCTCGCAGCGGCGCGC
GGCCCCGGACAAGCGCTGGGGATTCCCGTTTGAGGCGTCACTACTGTCAC
TGCCATCACCCCACGGAGCCACTTCTAGAGGGGAGTAGACCCGGCCCTTC
GCCGGGCAGAGAAGATGTTGCCCCTGTCCATCAAAGACGATGAATACAAA
CCACCCAAGTTCAATTTGTTCGGCAAGATCTCGGGCTGGTTTAGGTCTAT
ACTGTCCGACAAGACTTCCCGGAACCTGTTTTTCTTCCTGTGCCTGAACC
TCTCTTTCGCTTTTGTGGAACTACTCTACGGCATCTGGAGCAACTGCTTA
GGCTTGATTTCCGACTCTTTTCACATGTTTTTCGATAGCACTGCCATTTT
GGCTGGACTGGCAGCTTCTGTTATTTCAAAATGGAGAGATAATGATGCTT
TCTCCTATGGGTATGTTAGAGCGGAAGTTCTGGCTGGCTTTGTCAATGGC
CTATTTTTGATCTTCACTGCTTTTTTTATTTTCTCAGAAGGAGTTGAGAG
AGCATTAGCCCCTCCAGATGTCCACCATGAGAGACTGCTTCTTGTTTCCA
TTCTTGGGTTTGTGGTAAACCTAATAGGAATATTTGTTTTCAAACATGGA
GGTCATGGACATTCTCATGGCTCTGGCCACGGACACAGTCATTCCCTCTT
TAATGGTGCTCTAGATCAGGCACATGGCCATGTCGATCATTGCCATAGCC
ATGAAGTGAAACATGGTGCTGCACATAGCCATGATCATGCTCATGGACAT
GGACACTTTCATTCTCATGATGGCCCGTCCTTAAAAGAAACAACAGGACC
CAGCAGACAGATTTTACAAGGTGTATTTTTACATATCCTAGCAGATACAC
TTGGAAGTATTGGTGTAATTGCTTCTGCCATCATGATGCAAAATTTTGGT
CTGATGATAGCAGATCCTATCTGTTCAATTCTTATAGCCATTCTTATAGT
TGTAAGTGTTATTCCTCTTTTAAGAGAATCTGTTGGAATATTAATGCAGA
GAACTCCTCCCCTATTAGAAAATAGTCTGCCTCAGTGCTATCAGAGGGTA
CAGCAGTTGCAAGGAGTTTACAGTTTACAGGAACAGCACTTCTGGACTTT
ATGTTCTGACGTTTATGTTGGGACCTTGAAATTAATAGTAGCACCTGATG
CTGATGCTAGGTGGATTTTAAGCCACACATAATATTTTTACTCAGGCTGG
AGTGAGACAGCTCTACGTACAGATTGACTTTGCAGCCATGTAGTGAATGG
AAAGAAATTATGCACCTTTTATGGACCAAATTTTTCTGCCAGTAAGAATT
TCAGTTGTGGGCCTCCAGTCTTCTGGAATGTCTTCACTGCAGCTGCTGGA
AATCACTGCTTTCATTCCCACAAAACCAGTATTACTTTTTTTTAAAAAAA
GAAAGAAATTGGAAATCTGTGCTATACGTAATGTCATAATTGTTGACGTT
CTTCAGTATAATCATGTTTGTTAAATTGTTTGTACCTTGCAAACTTATGA
ACCMkACCATATTGGGTTTTCAAAGTGCCTTTGTATCCAAAAATGTATTT
GCCAGCATAGAAATGTACCATCAAGAGTCATGTATGTTTTATTTTTGTTT
TTATTTTTTATTTTTTGAGATGGAGTCTCGCTCTGTAGCCAGGCTGGAGT
GCAGTGGCATAATCTCAGCTCACTGCAACCTCTGCTTCCCGGGTTCAAGC
TATTCTCCTGCCTCAGCCTTCTGAGTAGCTGCGACTATGGGCGTGTGCCA
CCATGCCCAGCTAATTTTTGCATTTTTAGTAGAGATGGGGTTTCACCATG
TTGGCCAGGATGGTCTTGATCTCTTGACCTCGTGATCCGTCCACCTCAGC
CTCCCAAAGTGCTGGGATTACAGGTGTGAGCCACCACGCCCGGCTATGTT
TTTTTTTTTTTTTTTTTTTTAACAATGGATATTCTGTAAAATATCCTGTA
CAAAACAAGACTATTGCCAGTAATGTATACAAATTGTCTTTTTGTGTACT
TCCAGCATAGGTTTGGATATATGAACATTTTTCTTTTATTGTTTTATCTT
CACAGAAAATAAAAGGTGTTAATTTGCTTTTTAAAACAAATTTAATTATC
ACATTTTAAATTACTTTGTGGACTGTGTTTTCAAAACTTTGCAAATTCAT
CTGTTACACAGAAAATTTTGACTTAAAACATCTGCTGAATTCCAAATTTC
TGTAATAGCTACTGTATCTGTGATAAACTTTCCTATATCTTTCCTTGCCA
TTTCTATGGATTTTATAATGAAAGAAAAAGGCATTGGAGACTGAAGGCAG
AAATGGTTGTGACAGTGCTGTTTGGCTTTTTCATTCTTCAAATGCCAAGT
CATCCACTTTGTTTTCCTGTTTAGGCTTTGCACAAATACAATTGCTTTCA
GGAATCCTAAAGCAGCATTTTATTGAGTTTGAATTATTAAAGGTACAGAG
GAAATGTGGTGATGTAGAACTTTTCCTAACACAGGTATCTAGGAAGTAAG
TGCTGAGTTGATTTTCTAGGTTCTTACGTATTTGAAAAATAAAATTGCAA
TTCGAGATAAAAAAAAAAAAAAAAAGGGCGGCCGC.

The human 84234 sequence (SEQ ID NO:28) is approximately 2637 nucleotides long. The nucleic acid sequence includes an initiation codon (ATG) and a termination codon (TAG) which are underscored above. The region between and inclusive of the initiation codon and the termination codon is a methionine-initiated coding sequence of about 1131 nucleotides, including the termination codon (nucleotides indicated as “coding” of SEQ ID NO:28; SEQ ID NO:30). The coding sequence encodes a 376 amino acid protein (SEQ ID NO:29), which is recited as follows:

(SEQ ID NO:29)

MLPLSIKDDEYKPPKFNLFGKISGWFRSILSDKTSRNLFFFLCLNLSFAY
VELLYGIWSNCLGLISDSFHMFFDSTAILAGLAASVISKWRDNDAFSYGY
VRAEVLAGFVNGLFLWTAFFIFSEGVERALAPPDVHHERLLLVSILGFVV
NLIGIFVFKHGGHGHSHGSGHGHSHSLFNGALDQAHGHVDHCHSHEVKHG
AATTSHDHAHGHGHFHSHDGPSLKETTGPSRQILQGVFLHILADTLGSIG
VIASMMMQNFGLMTADPICSILIAILIVVSVTPLLRESVGILMQRTPPLL
ENSLPQCYQRVQQLQGVYSLQEQHFWTLCSDVYVGTLKLIVAPDADARWI
LSQTHNIFTQAGVRQLYVQIDFAAM.

### Example 2

### Tissue Distribution of 47476, 67210, 49875, 46842, 33201, 83378, 84233, 64708, 85041, or 84234 mRNA by TaqMan Analysis

Endogenous human 47476, 67210, 49875, 46842, 33201, 83378, 84233, 64708, 85041, or 84234 gene expression was determined using the Perkin-Elmer/ABI 7700 Sequence Detection System which employs TaqMan technology. Briefly, TaqMan technology relies on standard RT-PCR with the addition of a third gene-specific oligonucleotide (referred to as a probe) which has a fluorescent dye coupled to its 5′ end (typically 6-FAM) and a quenching dye at the 3′ end (typically TAMRA). When the fluorescently tagged oligonucleotide is intact, the fluorescent signal from the 5′ dye is quenched. As PCR proceeds, the 5′ to 3′ nucleolytic activity of Taq polymerase digests the labeled primer, producing a free nucleotide labeled with 6-FAM, which is now detected as a fluorescent signal. The PCR cycle where fluorescence is first released and detected is directly proportional to the starting amount of the gene of interest in the test sample, thus providing a quantitative measure of the initial template concentration. Samples can be internally controlled by the addition of a second set of primers/probe specific for a housekeeping gene such as GAPDH which has been labeled with a different fluorophore on the 5′ end (typically VIC).

To determine the level of 47476 mRNA in various human tissues a primer/probe set was designed. Total RNA was prepared from a series of human tissues using an RNeasy kit from Qiagen. First strand cDNA was prepared from 1 μg total RNA using an oligo-dT primer and Superscript II reverse transcriptase (Gibco/BRL). cDNA obtained from approximately 50 ng total RNA was used per TaqMan reaction. Tissues tested include the human tissues and several cell lines shown in Table 1.

TABLE 1

| | Relative |
|---|---|
| Tissue Type | Expression |
| Artery normal | 0 |
| Aorta diseased | 0 |
| Vein normal | 0 |
| Coronary SMC | 0 |
| HUVEC | 0 |
| Hemangioma | 0 |
| Heart normal | 0 |
| Heart CHF | 0 |
| Kidney | 0 |
| Skeletal Muscle | 0 |
| Adipose normal | 0 |
| Pancreas | 0 |
| primary osteoblasts | 0 |
| Osteoclasts (diff) | 0 |
| Skin normal | 0 |
| Spinal cord normal | 0 |
| Brain Cortex normal | 0 |
| Brain Hypothalamus normal | 0 |
| Nerve | 0 |
| DRG (Dorsal Root Ganglion) | 0 |
| Breast normal | 0 |
| Breast tumor | 0 |
| Ovary normal | 0 |
| Ovary Tumor | 0 |
| Prostate Tumor | 0 |
| Salivary glands | 0 |
| Colon normal | 0 |
| Colon Tumor | 0 |
| Lung normal | 0.2975 |
| Lung tumor | 0 |
| Lung COPD | 0.4021 |
| Liver normal | 0 |
| Liver fibrosis | 0 |
| Spleen normal | 0.2903 |
| Tonsil normal | 0 |
| Lymph node normal | 0 |
| Small intestine normal | 0 |
| Macrophages | 0 |
| Synovium | 0 |
| BM-MNC | 15.4634 |
| Activated PBMC | 0.0277 |
| Neutrophils | 2.7526 |
| Megakaryocytes | 3.0542 |
| Erythroid | 0.5418 |
| positive control | 0 |
| Prostate Normal | 0.0456 |
| Colon IBD | 0.0343 |

As shown in Table 1, 47476 mRNA was detected in bone marrow mononuclear cells (BM-MNC), megakaryocytes, neutrophils, erythroid cells, lung (normal and diseased, i.e., COPD (chronic obstructive pulmonary disease) and spleen.

To determine the level of 67210 mRNA in various human tissues a primer/probe set was designed. Total RNA was prepared from a series of human tissues using an RNeasy kit from Qiagen. First strand cDNA was prepared from 1 μg total RNA using an oligo-dT primer and Superscript II reverse transcriptase (Gibco/BRL). cDNA obtained from approximately 50 ng total RNA was used per TaqMan reaction. Tissues tested include the human tissues and several cell lines shown in Table 2.

TABLE 2

| | Relative |
|---|---|
| Tissue Type | Expression |
| Artery normal | 57.5117 |
| Aorta diseased | 38.7409 |
| Vein normal | 20.8328 |
| Coronary SMC | 102.2378 |
| HUVEC | 28.6564 |
| Hemangioma | 7.34 |
| Heart normal | 3.5573 |
| Heart CHF | 24.5183 |
| Kidney | 16.8046 |
| Skeletal Muscle | 31.5766 |
| Adipose normal | 9.7188 |
| Pancreas | 10.0268 |
| primary osteoblasts | 9.0054 |
| Osteoclasts (diff) | 0 |
| Skin normal | 6.9682 |
| Spinal cord normal | 8.4607 |
| Brain Cortex normal | 61.2138 |
| Brain Hypothalamus | 9.9575 |
| normal | |
| Nerve | 12.3873 |
| DRG (Dorsal Root | 23.5195 |
| Ganglion) | |
| Breast normal | 50.4151 |
| Breast tumor | 7.4167 |
| Ovary normal | 65.6073 |
| Ovary Tumor | 10.1667 |
| Prostate Normal | 9.4859 |
| Prostate Tumor | 18.7756 |
| Salivary glands | 7.8668 |
| Colon normal | 5.4482 |
| Colon Tumor | 45.2794 |
| Lung normal | 4.8932 |
| Lung tumor | 4.4407 |
| Lung COPD | 7.5989 |
| Colon IBD | 1.8866 |
| Liver normal | 0.908 |
| Liver fibrosis | 6.0243 |
| Spleen normal | 0.0988 |
| Tonsil normal | 1.5484 |
| Lymph node normal | 2.9604 |
| Small intestine normal | 5.0834 |
| Macrophages | 0 |
| Synovium | 15.5171 |
| BM-MNC | 0 |
| Activated PBMC | 0 |
| Neutrophils | 0 |
| Megakaryocytes | 0 |
| Erythroid | 0.412 |
| positive control | 44.8111 |

As shown in Table 2, 67210 mRNA was detected (average levels of expression greater than about 50) in coronary smooth muscle cells (SMC), normal artery, normal brain cortex, normal breast, and normal ovary. The highest level of 67210 expression was found in coronary SMC.

To determine the level of 49875 mRNA in various human tissues a primer/probe set was designed. Total RNA was prepared from a series of human tissues using an RNeasy kit from Qiagen. First strand cDNA was prepared from 1 μg total RNA using an oligo-dT primer and Superscript II reverse transcriptase (Gibco/BRL). cDNA obtained from approximately 50 ng total RNA was used per TaqMan reaction. Tissues tested include the human tissues shown in Table 3.

TABLE 3

| | Relative |
|---|---|
| Tissue Type | Expression |
| Breast N | 34.92 |
| Breast N | 24.77 |
| Breast N | *173.74 |
| Breast T | 24.10 |
| Breast T | 7.63 |
| Breast T | 23.85 |
| Breast T | 5.34 |
| Breast T | 21.87 |
| Breast T | 101.18 |
| Breast T | 12.30 |
| Breast T | *178.01 |
| Ovary N | 12.09 |
| Ovary N | 10.49 |
| Ovary N | 20.55 |
| Ovary T | 13.79 |
| Ovary T | 44.81 |
| Ovary T | 17.64 |
| Ovary T | 12.87 |
| Ovary T | 63.37 |
| Ovary T | 85.38 |
| Ovary T | *129.41 |
| Lung N | 1.25 |
| Lung N | 12.05 |
| Lung N | 3.31 |
| Lung N | 1.52 |
| Lung T | 34.08 |
| Lung T | 93.10 |
| Lung T | 16.52 |
| Lung T | 14.28 |
| Lung T | 31.58 |
| Lung T | 20.83 |
| Lung T | *130.31 |
| Lung T | 9.62 |
| Colon N | 4.58 |
| Colon N | 5.34 |
| Colon N | 19.78 |
| Colon N | 0.71 |
| Colon T | **145.59 |
| Colon T | 48.19 |
| Colon T | 83.62 |
| Colon T | 12.56 |
| Colon T | 30.29 |
| Colon T | 20.33 |
| Colon T | 45.75 |
| Colon T | 10.27 |
| Liver N | 1.74 |
| Liver N | 36.65 |
| Liver Met | 10.03 |
| Liver Met | 47.86 |
| Liver Met | 99.10 |
| Liver Met | 24.52 |
| A4 HMVEC-Arrested | 2.31 |
| C48 HMVEC-Proliferating | 14.28 |
| CHT 50 Placenta | 103.66 |
| ONC 102 Hemangioma | 5.84 |

*β2 Ct and target Ct values very close
**∂∂Ct value less than 3

As shown in Table 3, 49875 mRNA was detected in normal (N) or tumor (T) breast, ovary, lung, and colon tissue samples. Significantly, 49875 mRNA was upregulated in a subset of ovary, lung, and colon tumors, as compared to the respective normal samples. This suggests that 49875 molecules play a role in tumor development, and that they can be used for diagnosis of ovary, lung, and colon tumors. 49875 mRNA was also detected in placenta tissue samples and in normal liver (N), as well as metastatic liver (Met), samples. Again, 49875 mRNA was upregulated in a subset of the metastatic liver samples, as compared to the normal liver tissue.

### Example 3

### Tissue Distribution of 47476, 67210, 49875, 46842, 33201, 83378, 84233, 64708, 85041, or 84234 mRNA by Northern Analysis

Northern blot hybridizations with various RNA samples can be performed under standard conditions and washed under stringent conditions, i.e., 0.2× SSC at 65° C. A DNA probe corresponding to all or a portion of the 47476, 67210, 49875, 46842, 33201, 83378, 84233, 64708, 85041, or 84234 cDNA (SEQ ID NO:1, SEQ ID NO:4, SEQ ID NO:7, SEQ ID NO:10, SEQ ID NO:13, SEQ ID NO:16, SEQ ID NO:19, SEQ ID NO:22, SEQ ID NO:25, or SEQ ID NO:28) can be used. The DNA was radioactively labeled with ³²P-dCTP using the Prime-It Kit (Stratagene, La Jolla, Calif.) according to the instructions of the supplier. Filters containing mRNA from mouse hematopoietic and endocrine tissues, and cancer cell lines (Clontech, Palo Alto, Calif.) can be probed in ExpressHyb hybridization solution (Clontech) and washed at high stringency according to manufacturer's recommendations.

### Example 4

### Recombinant Expression of 47476, 67210, 49875, 46842, 33201, 83378, 84233, 64708, 85041, or 84234 in Bacterial Cells

In this example, 47476, 67210, 49875, 46842, 33201, 83378, 84233, 64708, 85041, or 84234 is expressed as a recombinant glutathione-S-transferase (GST) fusion polypeptide in E. coli and the fusion polypeptide is isolated and characterized. Specifically, 47476, 67210, 49875, 46842, 33201, 83378, 84233, 64708, 85041, or 84234 is fused to GST and this fusion polypeptide is expressed in E. coli, e.g., strain PEB 199. Expression of the GST-47476, 67210, 49875, 46842, 33201, 83378, 84233, 64708, 85041, or 84234 fusion protein in PEB199 is induced with IPTG. The recombinant fusion polypeptide is purified from crude bacterial lysates of the induced PEB 199 strain by affinity chromatography on glutathione beads. Using polyacrylamide gel electrophoretic analysis of the polypeptide purified from the bacterial lysates, the molecular weight of the resultant fusion polypeptide is determined.

### Example 5

### Expression of Recombinant 47476, 67210, 49875, 46842, 33201, 83378, 84233, 64708, 85041, or 84234 Protein in COS Cells

To express the 47476, 67210, 49875, 46842, 33201, 83378, 84233, 64708, 85041, or 84234 gene in COS cells (e.g., COS-7 cells, CV-1 origin SV40 cells; Gluzman (1981) CellI23:175-182), the pcDNA/Amp vector by Invitrogen Corporation (San Diego, Calif.) is used. This vector contains an SV40 origin of replication, an ampicillin resistance gene, an E. coli replication origin, a CMV promoter followed by a polylinker region, and an SV40 intron and polyadenylation site. A DNA fragment encoding the entire 47476, 67210, 49875, 46842, 33201, 83378, 84233, 64708, 85041, or 84234 protein and an HA tag (Wilson et al. (1984) Cell 37:767) or a FLAG tag fused in-frame to its 3′ end of the fragment is cloned into the polylinker region of the vector, thereby placing the expression of the recombinant protein under the control of the CMV promoter.

To construct the plasmid, the 47476, 67210, 49875, 46842, 33201, 83378, 84233, 64708, 85041, or 84234 DNA sequence is amplified by PCR using two primers. The 5′ primer contains the restriction site of interest followed by approximately twenty nucleotides of the 47476, 67210, 49875, 46842, 33201, 83378, 84233, 64708, 85041, or 84234 coding sequence starting from the initiation codon; the 3′ end sequence contains complementary sequences to the other restriction site of interest, a translation stop codon, the HA tag or FLAG tag and the last 20 nucleotides of the 47476, 67210, 49875, 46842, 33201, 83378, 84233, 64708, 85041, or 84234 coding sequence. The PCR amplified fragment and the pcDNA/Amp vector are digested with the appropriate restriction enzymes and the vector is dephosphorylated using the CIAP enzyme (New England Biolabs, Beverly, Mass.). Preferably the two restriction sites chosen are different so that the 47476, 67210, 49875, 46842, 33201, 83378, 84233, 64708, 85041, or 84234 gene is inserted in the correct orientation. The ligation mixture is transformed into E. coli cells (strains HB101, DH5α, SURE, available from Stratagene Cloning Systems, La Jolla, Calif., can be used), the transformed culture is plated on ampicillin media plates, and resistant colonies are selected. Plasmid DNA is isolated from transformants and examined by restriction analysis for the presence of the correct fragment.

COS cells are subsequently transfected with the 47476, 67210, 49875, 46842, 33201, 83378, 84233, 64708, 85041, or 84234-pcDNA/Amp plasmid DNA using the calcium phosphate or calcium chloride co-precipitation methods, DEAE-dextran-mediated transfection, lipofection, or electroporation. Other suitable methods for transfecting host cells can be found in Sambrook, J., Fritsh, E. F., and Maniatis, T. (1989) Molecular Cloning: A Laboratory Manual. 2nd, ed., Cold Spring Harbor Laboratory, Cold Spring Harbor Laboratory Press, Cold Spring Harbor, N.Y. The expression of the 47476, 67210, 49875, 46842, 33201, 83378, 84233, 64708, 85041, or 84234 polypeptide is detected by radiolabelling (³⁵S-methionine or ³⁵S-cysteine available from NEN, Boston, Mass., can be used) and immunoprecipitation (Harlow, E. and Lane, D. (1988) Antibodies: A Laboratory Manual, Cold Spring Harbor Laboratory Press, Cold Spring Harbor, N.Y.) using an HA specific monoclonal antibody. Briefly, the cells are labeled for 8 hours with ³⁵S-methionine (or ³⁵S-cysteine). The culture media are then collected and the cells are lysed using detergents (RIPA buffer, 150 mM NaCl, 1% NP-40, 0.1% SDS, 0.5% DOC, 50 mM Tris, pH 7.5). Both the cell lysate and the culture media are precipitated with an HA specific monoclonal antibody. Precipitated polypeptides are then analyzed by SDS-PAGE.

Alternatively, DNA containing the 47476, 67210, 49875, 46842, 33201, 83378, 84233, 64708, 85041, or 84234 coding sequence is cloned directly into the polylinker of the pcDNA/Amp vector using the appropriate restriction sites. The resulting plasmid is transfected into COS cells in the manner described above, and the expression of the 47476, 67210, 49875, 46842, 33201, 83378, 84233, 64708, 85041, or 84234 polypeptide is detected by radiolabelling and immunoprecipitation using a 47476, 67210, 49875, 46842, 33201, 83378, 84233, 64708, 85041, or 84234 specific monoclonal antibody.

### EXAMPLES FOR 21617 AND 55562

### Example 6

### Identification and Characterization of Human 21617 or 55562 cDNA

The human 21617 nucleic acid sequence is recited as follows:

(SEQ ID NO:63)

TAGTCTAACTCGCGGCTGTCACCGCCACTGCAGCGGAGCCGGCCGGCCGG
GCGCTGCGGGACGGGCGGGCGGCTGCCGGCAGGAGGCGCCGAGCCGGGTG
ACTGCCGCGGCGGGCACAGTCCGGGGCCACAGCGCCGAGCCCGGGCGGGA
GTGGCCCCGCGCAGGCAGGGAGCGGCGCCGCGCACTCCAACCCGGCGGGC
ACCTCGGGGGCGGGCGCGGGGCGCAGCCTTCTCGTCCCGGCCTCTGTGAC
AAGCGCCCCGGAGCCGGGAGCCCGATTGCCGGGCTCGGGGTGGGCGCGGA
CGCAGGCACTGGGCTCGTGCGGGGCCCCGGGCGTCGCGATGGAACATCGT
GGTGGAGTTCTTCGTGGTCACTTTCAAAGTGCTCTGGGCGTTCGTGCTGG
CCGCGGCGCGCTGGCTGGTGCGGCCCAAGGAGAAGAGCGTGGCGGGCCAG
GTGTGCCTCATCACCGGCGCCGGCAGCGGCCTGGGCCGCCTCTTCGCGCT
GGAGTTCGCCCGGCGTCGGGCGCTGCTGGTGCTGTGGGACATCAACACGC
AAAGCAACGAGGAGACGGCTGGCATGGTGCGCCACATCTACCGCGACCTG
GAGGCGGCCGACGCCGCTGCGCTGCAAGCTGGGAATGGTGAGGAAGAAAT
TCTGCCCCACTGTAACTTGCAGGTTTTTACCTACACCTGTGACGTGGGGA
AGAGGGAGAACGTCTACCTGACGGCTGAAAGAGTCCGCAAGGAGGTTGGC
GAAGTCTCAGTCCTGGTCAATAATGCTGGTGTGGTCTCTGGGCATCACCT
TCTGGAATGTCCTGATGAGCTCATTGAGAGAACCATGATGGTCAATTGCC
ATGCACACTTCTGGACCACTAAGGCTTTTCTTCCTACGATGCTGGAGATT
AATCATGGTCATATTGTGACAGTTGCAAGTTCCTTGGGATTGTTCAGTAC
TGCCGGAGTTGAGGATTACTGTGCCAGTAAATTTGGAGTTGTGGGTTTTC
ATGAATCCCTGAGCCATGAACTAAAGGCTGCTGAAAAGGATGGAATTAAA
ACAACCTTGGTTTGCCCTTATCTTGTAGACACTGGCATGTTCAGAGGCTG
CCGAATCAGGAAAGAAATTGAGCCTTTTCTGCCACCTCTGAAGCCTGATT
ACTGTGTGAAGCAGGCCATGAAGGCCATCCTCACTGACCAGCCCATGATC
TGCACTCCCCGCCTCATGTACATCGTGACCTTCATGAAGAGCATCCTACC
ATTTGAAGCAGTTGTGTGCATGTATCGGTTCCTAGGAGCGGACAAGTGTA
TGTACCCCTTTATTGCTCAAAGAAAGCAAGCCACAAACAATAATGAAGCA
AAAAATGGAATCTAAGAATCTTTTTGTATGGAATATTACTTCTATCAGAA
GATGATCAAGATGTTTCAGTCCAGTGCACATCAGCATTGCTGACATTTTA
TGGATTCTAAACTTGTGTTGTTTCTTTTTTAAATCAACTTTTTAAAAAAA
TAAAGTGTAAATTAACCGACTAGAGTACTTGGAAAATGTGATCAGTACAA
GTGAACTTAGGTTGTTGCCAACAGGGTCCTTTTAGGCAGAACCCAGAAAC
CAGTCAAATCTGTAGAGAAGCAGTGTGACATCTTCAGGTTACCATTATTT
TTTAATGAGCAGGAAGTCTAGAAATGATAACTAGACTGTATGTTTCATGT
GTGTGATTTTTCAGAATTCCCAGAGTTTACTCATTCTTGTTATTAAACTC
TAGCCAGTTGACATCTTCGCAATTTCAAGGACTGATAGTGCTGTATTTTC
TCACGTTTTCTAAGTTTCCGTTTTGCAAGGCCTAGGTGACTTTTTCATGG
TGTTTGTATGTTTAGCTCTTTTGAAAAGGATTTTGAATCTCCATCAACTG
AAGTAAATGATGTCTGAGTGTTACAGTWAAGGTGACCAAGTCTCTTTCTT
AAAGTCACAATGACTAAAGTATTAGTTGAATTTTTTTTTTTTTTTTTGAT
GGAGTCTCGCTCTGTCACCAGGCTGGAGTGCAGTAGCACAATCACGGCTC
ACTGCAATCTCTGCCTCCCRGTTTCAAGTGATTCTGCTGTCTCAGCCTCC
CAAGTAGCTGGGACTACAGGCATGCGCCACCACGCCCAGCTAATTTTTGT
ATTTTTAGTAGAGACGGGGTTTCACCATGTTGGTCAGGATGGTCTCCATC
TCTTGACATTGTGATCCACCTGCCTCGGCCTCCCAAAGTGCTGGGATTAC
AGGCATGAGCCACTGCACCCAGCCTTGAATTTTTAATTTTATCTCTGATA
TACTTCATTAAGTGTCTGGAGACCTAATTATCCTAAAAGATCATACATTT
TCTACCTATGAATTTTGCTGCATACAGAAAGTGCCCTTTCCTCAGGAAGT
TGCTGTGTTTCATTTCTTTGGATGGACTCTTATCTAGAATACATAGCAGC
TCTGCAAAGAAACAGTTTTTAAAAATGGGAACTTCTACATTGAAAAGTCC
CCATTTTTGTGCCAACTATGATTAGTGAGAGGAAGAAATCTTATTCTATG
GCATATGTATGGAAGGGTGTAAAGATTCTTTTGAAAGGTTTATTCACATT
GTAGAACAGCAAATGACATTTTTACAGTATTTTTTTGTAAAGCAAACTAT
TTTGTGCCTTGAATTTGGTATATGTGTATTAGTGAAACATTGTAAAGGTG
AACTTCTACCTCTGTATCTAAATGTATACCATCCACTTGTAAATGACTAT
AAACTATTATGTGATTGCTTTTTTTTTTAGAATGTCTTGTTTAAATAGTG
GCCAATGTTTAAGGCTGTTAAAATAAGCCAACTTTTACTAATTGGGGAGT
TTTATAAATGACTGATTAAATTTAAAGAATTAACTTACATGCAATTGTGT
GATTATTAGTTATCAGCAGTGTTGTAAGGAAAATTATTGTGTTTTTTTTT
ATGATCATTATCCCACTTTAGGTAAAGAAAAATATTGGAATGGAATAGTG
TTGGGAAACAGACATTAACAACCTAGGGTGCCTGCACTCAAATAGCCGAT
GTTACTGTCCCTAGATTAGAGACTTGATTAAGGGCTTGTTTGTACCAAAA
GTGGGGAAACAATGCCATGACCTGTGTTTTAGTTTGGCTGCACCACAGAT
CAAATCTGCACTGTGTCTACATATAGGAAAGGTCCTGGTGTGTGCTAATG
TTCCCAATGCAGGACTTGAGGAAGAGCTCTGTTATATGTTTCCATTTCTC
TTTATCAAAGATAACCAAACCTTATGGCCCTTATAACAATGGAGGCACTG
GCTGCCTCTTAATTTTCAATCATGGACCTAAAGAAGTACTCTGAAGGGTC
TCAACAATGCCAGGTGGGGACAGATATACTCAGAGATTATCCAGGTCTGC
CTCCCAGCGAGCCTGGAGTACACCAGACCCTCCTAGAGAAATCTGTTATA
ATTTAACAACCCACTTATCCACCTTAAAACTGAGGAAAGTCGTCTTTACA
TCTAATTTTATTCTTGTGTGTTATAACTTAAACCTATTTCTATTTTTGTT
TGTTATTGCCCTTATAAGGGTGTCCATCTCCAAGTTCAATAAACTAATTC
ATTTAAAAAAAAAAAAAAAAAAA.

The human 21617 sequence (SEQ ID NO:63) is approximately 3624 nucleotides long. The nucleic acid sequence includes an initiation codon (ATG) and a termination codon (TAA), which are indicated in bold and underscored above. The region between and inclusive of the initiation codon and the termination codon is a methionine-initiated coding sequence of about 1026 nucleotides, including the termination codon (nucleotides 339 to 1364 of SEQ ID NO:63, SEQ ID NO:65). The coding sequence encodes a 341 amino acid protein (SEQ ID NO:64), which is recited as follows:

(SEQ ID NO:64)

MNIVVEFFVVTFKVLWAFVLAAARWLVRPREKSVAGQVCLITGAGSGLGR
LFALEFARRRALLVLWDINTQSNEETAGMVRHIYRDLEAADAAALQAGNG
EEEILPHCNLQVFTYTCDVGKRENVYLTAERVRKIEVGEVSVLVNNAGVV
SGHHLLECPDELIERTMMVNCHAHFWTTKFLPTMLEINTIGHWTVASSLG
LFSTAGVEDYCASKYGVVGFHESLSHELKAAEKDGIKTTLVCPYLVDTGM
FRGCRRKEIEPFLPPLKPDYCvKQAMKAILTDQPMICTPRLMYIVTFMKS
ILPFEAVVCMYRFLGADKCMYPFIAQRKQATNNNEAKNGI.

The human 55562 nucleic acid sequence is recited as follows:

(SEQ ID NO:66)

CCTGCTGCAATGGCTTACGGGAGCCAATGTGACGGGATCAGGGCAGACCC
ATTTAGGGTTTCGTAACCGGCCAATTCAGTACGCAATAGGGAAAATCAAT
TAGGATCTGCAGAGGGTTCCCGGATACACCTTGCGAAGAATGCCGCACTC
TCCGCCACTCATTCCCCACTCACCGGCACCCGCTAAACCTTCAGCCTGAA
TTTTCCTCCGAAGGGAAGCAGAGCAGAGGAAGAACTACCAAGTGCTACAC
TCAAAGCCTGCCGTCGCAGTGAGCGCGACCTCCAAACTGAGGCATTTTTG
TTCCGGCGAAATCCCTCCCACTCAGGAAAGTCCCTAGAAAGAGAGCGCAG
GCGCCTGGGGTATCACATGACCACTTCCCGGAAGCGCAGCAGACCCGCTC
AACTTCATCCTGGGTTGAGGCGGAGGAGAACTTCCAGAATTATGGCGAAG
TCCGGGCTGAGGCAGGACCCGCAGAGCACAGCTGCAGCCACTGTGCTAAA
AGCGGGCAGTAGAACTAGATTCGGAGTCGCGGTATCCGCAGGCTCTGGTG
TGTTACCAAGAGGGGATTGATCTGCTCCTGCAGGTTCTGAAAGGTACCAA
AGATAATACTAAGAGATGTAATCTCAGAGAAAAAATTTCCAAATACATGG
ACAGAGCGGAAAACATAAAGAAGTACTTGGACCAAGAAAAAGAAGATGGA
AAATATCACAAGCAAATTAAAATAGAAGAGAATGCAACAGGTTTCAGTTA
TGAGTCACTTTTTCGCGAATACCTTAATGAGACAGTTACAGAAGTTTGGA
TAGAAGATCCTTATATTAGACATACTCATCAGCTGTATAACTTTCTTCGA
TTTTGTGAGATGCTTATTAAGAGACCATGTAAAGTAAAAACTATTCACCT
TCTCACCTCTCTGGATGAAGGCATTGAGCAAGTGCAGCAAAGTAGAGGCC
TGCAAGAAATAGAAGAGTCACTCAGGAGTCACGGAGTGCTGTTGGAAGTT
CAATACTCTTCTTCAATACATGACCGAGAAATTAGGTTCAACAATGGATG
GATGATTAAGATTGGAAGGGGACTTGATTATTTTAAGAAACCACAGAGTC
GTTTTTCCCTTGGATATTGTGATTTTGATTTAAGACCATGTCATGAAACA
ACAGTAGACATTTTTCATAAGAAGCATACAAAAAATATATGATGGGTGGT
AGCCTAATTTGTATTATGTCTACTTTAAGTGAATATTGGATTTTTTTTAA
AAGATCACTTTTATAATGTATGAATTTAACAATAAACTTTTATATTTCTA
CTAAAAAAAAAAAAAAAAAAAAAAAAA.

The human 55562 sequence (SEQ ID NO:66) is approximately 1327 nucleotides long. The nucleic acid sequence includes an initiation codon (ATG) and a termination codon (TGA), which are indicated in bold and underscored above. The region between and inclusive of the initiation codon and the termination codon is a methionine-initiated coding sequence of about 825 nucleotides, including the termination codon (nucleotides 367 to 1191 of SEQ ID NO:66; SEQ ID NO:68). The coding sequence encodes a 274 amino acid protein (SEQ ID NO:67), which is recited as follows:

(SEQ ID NO:67)

MTTSRKRSRPAQLHPGLRRRRTSRIMAKSGLRQDPQSTAAATVLKRAVEL
DSESRYPQALVCYQEGIDLLLQVLKGTKDNTKRCNLREKISKYMDRAENI
KKYLDQEKIDGKYHKQIKIEENATGFSYESLFREYLNETVTEVWIEDPYT
RHTHQLYNFLRFCEMLIKRPCKVKTIHLLTSLDEGIEQVQQSRGLQEIEE
SLRSHGVLLEVQYSSSHDREIRFNNGWMIKIGRGLDYFKKPQSRFSLGYC
DFDLRPCHETTVDWHKKHTKNI.

### Example 7

### Tissue Distribution of 21617 or 55562 mRNA

Northern blot hybridizations with various RNA samples can be performed under standard conditions and washed under stringent conditions, i.e., 0.2× SSC at 65° C. A DNA probe corresponding to all or a portion of the 21617 or 55562 cDNA (SEQ ID NO:63 or SEQ ID NO:66, respectively) can be used. The DNA can be radioactively labeled with ³²P-dCTP using the Prime-It Kit (Stratagene, La Jolla, Calif.) according to the instructions of the supplier. Filters containing mRNA from mouse hematopoietic and endocrine tissues, and cancer cell lines (Clontech, Palo Alto, Calif.) can be probed in ExpressHyb hybridization solution (Clontech) and washed at high stringency according to manufacturer's recommendations.

Endogenous human 21617 or 55562 gene expression can also be determined using the Perkin-Elmer/ABI 7700 Sequence Detection System which employs TaqMan technology. Briefly, TaqMan technology relies on standard RT-PCR with the addition of a third gene-specific oligonucleotide (referred to as a probe) which has a fluorescent dye coupled to its 5′ end (typically 6-FAM) and a quenching dye at the 3′ end (typically TAMRA). When the fluorescently tagged oligonucleotide is intact, the fluorescent signal from the 5′ dye is quenched. As PCR proceeds, the 5′ to 3′ nucleolytic activity of Taq polymerase digests the labeled primer, producing a free nucleotide labeled with 6-FAM, which is now detected as a fluorescent signal. The PCR cycle where fluorescence is first released and detected is directly proportional to the starting amount of the gene of interest in the test sample, thus providing a quantitative measure of the initial template concentration. Samples can be internally controlled by the addition of a second set of primers/probe specific for a housekeeping gene such as GAPDH which has been labeled with a different fluorophore on the 5′ end (typically VIC).

To determine the level of 21617 in various human tissues a primer/probe set was designed. Total RNA was prepared from a series of human tissues using an RNeasy kit from Qiagen. First strand cDNA was prepared from 1 μg total RNA using an oligo-dT primer and Superscript II reverse transcriptase (Gibco/BRL). cDNA obtained from approximately 50 ng total RNA was used per TaqMan reaction. Tissues tested include the human tissues and several cell lines shown in Tables 4-8. 21617 mRNA was detected in colon cancer cell lines and samples (Tables 4, 5, 8). 21617 mRNA expression was also found in breast, lung, and cervical carcinoma cell lines (Tables 4-8).

TABLE 4
In vitro Expression in Synchronized Cell Cycle Panel

| Tissue Type | Expression |
|---|---|
| HCT 116 Aphidl t = 0 | 63.6 |
| HCT 116 Aphidl t = 3 | 66.3 |
| HCT 116 Aphidl t = 6 | 43.0 |
| HCT 116 Aphidl t = 9 | 70.3 |
| HCT 116 Aphidl t = 12 | 57.1 |
| HCT 116 Aphidl t = 15 | 39.4 |
| HCT 116 Aphidl t = 18 | 57.1 |
| HCT 116 Aphidl t = 21 | 65.2 |
| HCT 116 Aphidl t = 24 | 58.9 |
| HCT 116 Noc t = 0 | 78.8 |
| HCT 116 Noc t = 3 | 92.5 |
| HCT 116 Noc t = 6 | 90.6 |
| HCT 116 Noc t = 9 | 75.1 |
| HCT 116 Noc t = 15 | 86.0 |
| HCT 116 Noc t = 18 | 89.6 |
| HCT 116 Noc t = 21 | 56.9 |
| HCT 116 Noc t = 24 | 66.5 |
| DLD noc t = 0 | 105.5 |
| DLD noc t = 3 | 236.5 |
| DLD noc t = 6 | 216.1 |
| DLD noc t = 9 | 251.7 |
| DLD noc t = 12 | 1117.3 |
| DLD noc t = 15 | 129.4 |
| DLD noc t = 18 | 196.1 |
| DLD noc t = 21 | 170.8 |
| A549 Mimo t = 0 | 110.3 |
| A549 Mimo t = 3 | 160.4 |
| A549 Mimo t = 6 | 64.5 |
| A549 Mimo t = 9 | 54.4 |
| A549 Mimo t = 15 | 48.5 |
| A549 Mimo t = 18 | 62.7 |
| A549 Mimo t = 21 | 53.7 |
| A549 Mimo t = 24 | 69.1 |
| MCF10A Mimo t = 0 | 110.0 |
| MCF10A Mimo t = 3 | 73.6 |
| MCF10A Mimo t = 6 | 49.4 |
| MCF10A Mimo t = 9 | 62.7 |
| MCF10A Mimo t = 12 | 65.8 |
| MCF10A Mimo t = 18 | 42.0 |
| MCF10A Mimo t = 21 | 31.8 |
| MCF10A Mimo t = 24 | 25.0 |

Expression of 21617 mRNA in synchronized cells grown in culture is shown in Table 4. Colon cancer cell lines HCT 116 and DLD, human lung carcinoma cell line A549 and human mammary epithelial cell line MCF10A all show expression of 21617 mRNA. The highest level of expression is shown at the mid pint of the cell cycle in DLD cells (colorectal carcinoma cell line).

TABLE 5
21617 Expression In Colon Metastasis Panel

| Tissue Type | Expression |
|---|---|
| CHT 371 Colon N | 0.45 |
| CHT 523 Colon N | 0.10 |
| NDR 104 Co1on N | 0.16 |
| CHT 520 Colonic ACA-C | 0.41 |
| CHT 1365 Colonic ACA-C | 0.04 |
| CHT 382 Colonic ACA-B | 2.76 |
| CHT 122 Adenocarcinoma | 0.91 |
| CHT 077 Liver-Colon Mets | 2.76 |
| CHT 739 Liver-Colon Mets | 0.79 |
| CHT 755 Liver-Colon Mets | 6.43 |
| CHT001 Liver-Colon Mets | 2.90 |
| CHT 084 Liver-Colon Mets | 1.50 |
| CHT 113 Liver-Colon Mets | 0.16 |
| CHT 114 Liver-Colon Mets | 35.65 |
| CHT 127 Liver-Colon Mets | 4.07 |
| CHT 137 Liver-Colon Mets | 2.07 |
| CHT 218 Liver-Colon Mets | 0.13 |
| CHT 220 Liver-Colon Mets | 1.98 |
| CHT 324 Liver-Colon Mets | 0.54 |
| CHT 340 Liver-Colon Met | 7.24 |
| CHT 530 Liver-Colon Met | 0.65 |
| CHT 849 Liver-Colon Met | 4.76 |
| CHT 1637 Liver-Colon Met | 1.46 |
| CHT 131 Liver-Colon Met | 11.72 |
| NDR 165 Liver Normal | 0.79 |
| NDR 150 Liver Normal | 1.80 |
| PIT 236 Liver Normal | 1.00 |

Expression of 21617 mRNA in a colon tumor metastasis panel is shown in Table 5. One of the colon cancer cell lines displays elevated expression of 21617 mRNA, while a subset of the Liver-Colon metastases express elevated levels of 21617 mRNA, suggesting that 21617 is a marker of cancer of the colon and liver-colon metastases. The highest level of expression in found in a liver metastasis sample.

TABLE 6
21617 Expression in Expanded Breast Panel

| Tissue Type | Expression |
|---|---|
| CHT 2242 Breast Normal | 0.00 |
| CHT 2251 Breast Normal | 2.80 |
| NDR824 Breast Normal | 2.68 |
| CHT 1744 Breast-ILC | 3.77 |
| NDR 133 Breast-ILC | 4.58 |
| CLN 662 Breast-ILC | 0.84 |
| CHT 1985 Breast-ILC | 0.34 |
| CLN 658 Breast-AC IDC II | 1.74 |
| CLN 732 Breast-AC IDC II | 4.52 |
| CHT 1828 Breast-Tumor IDC II | 0.15 |
| CHT 2012 Bresat-Tumor IDC II | 0.01 |
| CLN 1026 Breast-AC IDC II | 2.77 |
| CLN 1027 Breast-AC IDC II | 1.29 |
| CHT1782 Breast-Tumor IDC III | 6.50 |
| CHT1784 Breast-Tumor IDC III | 27.30 |
| CHT1786 Breast-Tumor IDC III | 0.78 |
| CLN 1023 Breast-AC IDC III | 1.38 |
| CLN 1024 Breast-AC IDC III | 0.50 |
| PIT 058 Lung-Breast Met | 0.00 |
| PIT 116 Lung-Breast Met | 0.33 |
| CHT841 LN-Breast Met | 0.00 |
| CLN 425 LN-Breast Met | 0.04 |
| PIT 059 Liver-Breast Met | 0.87 |
| PIT 236 Liver N | 4.63 |
| PIT 260 Liver N | 0.06 |
| PIT 207 Lung N | 0.87 |
| PIT 298 Lung N | 0.07 |
| Pooled LN normal | 12.01 |
| CHT 2248 Breast Normal | 23.60 |

Table 6 shows 21617 mRNA expression in an Expanded Breast Panel.

TABLE 7
21617 Expression in Oncology Phase II Panel

| Tissue Type | Expression |
|---|---|
| PIT 400 Breast N | 0.00 |
| PIT 372 Breast N | 0.00 |
| CHT 1228 Breast N | 0.00 |
| MDA 304 Breast T: MD-IDC | 0.00 |
| CHT 2002 Breast T: IDC | 0.00 |
| MDA 236-Breast T: PD-IDC(ILC?) | 0.00 |
| CHT 562 Breast T: IDC | 3.44 |
| NDR 138 Breast T ILC (LG) | 6.90 |
| CHT 1841 Lymph node (Breast met) | 0.00 |
| PIT 58 Lung (Breast met) | 0.00 |
| CHT 620 Ovary N | 0.00 |
| CHT 619 Ovary N | 0.00 |
| CLN 012 Ovary T | 0.00 |
| CLN 07 Ovary T | 0.00 |
| CLN 17 Ovary T | 0.00 |
| MDA 25 Ovary T | 0.00 |
| CLN 08 Ovary T | 0.00 |
| PIT 298 Lung N | 0.00 |
| MDA 185 Lung N | 0.00 |
| CLN 930 Lung N | 0.00 |
| MPI 215 Lung T—SmC | 0.43 |
| MDA 259 Lung T-PDNSCCL | 30.93 |
| CHT 832 Lung T-PDNSCCL | 1.26 |
| MDA 262 Lung T-SCC | 5.96 |
| CHT 793 Lung T-ACA | 0.20 |
| CHT 331 Lung T-ACA | 0.00 |
| CHT 405 Colon N | 0.00 |
| CHT 1685 Colon N | 0.00 |
| CHT 371 Colon N | 0.01 |
| CHT 382 Colon T: MD | 0.23 |
| CHT 528 Colon T: MD | 0.12 |
| CLN 609 Colon T | 0.18 |
| NDR 210 Colon T: MD-PD | 0.82 |
| CHT 340 Colon-Liver Met | 4.14 |
| CHT l637 Colon-Liver Met | 0.61 |
| PIT 260 Liver N (female) | 0.10 |
| CHT 1653 Cervix Squamous CC | 13.94 |
| CHT 569 Cervix Squamous CC | 0.00 |
| A24 HMVEC-Arr | 0.96 |
| C48 HMVEC-Prol | 0.10 |
| Pooled Hemangiomas | 0.00 |
| HCT116N22 Normoxic | 7.84 |
| HCT116H22 Hypoxic | 2.02 |

Table 7 shows 21617 mRNA expression in an oncology phase II panel. The highest level of expression was found in lung tumor and cervical squamous carcinoma. In addition, elevated expression of 21617 mRNA was detected in a subset of breast (IDC and ILC) and lung tumor (PDNSCCL and SCC) samples as compared to normal breast and lung tissue. Expression of 21617 mRNA was also detected in human vascular endothelial cells (HMVECs).

Table 8
21617 Expression in Xenograft Panel

| Tissue Type | Expression |
|---|---|
| MCF-7 Breast T | 16.63 |
| ZR75 Breast T | 33.84 |
| T47D Breast T | 24.43 |
| MDA 231 Breast T | 0.24 |
| MDA 435 Breast T | 1.58 |
| SKBr3 Breast | 75.10 |
| DLD 1 ColonT (stageC) | 138.22 |
| SW480 Colon T (stage B) | 6.99 |
| SW620 ColonT (stageC) | 92.46 |
| HCT116 | 19.37 |
| HT29 | 4.38 |
| Colo 205 | 32.69 |
| NCIH125 | 9.55 |
| NCIH67 | 83.33 |
| NCIH322 | 50.42 |
| NCIH460 | 1.95 |
| A549 | 11.92 |
| NHBE | 35.40 |
| SKOV-3 ovary | 6.87 |
| OVCAR-3 ovary | 8.37 |
| 293 Baby Kidney | 27.97 |
| 293T Baby Kidney | 113.05 |

Table 8 shows 21617 mRNA expression in a xenograft panel. Stage C colon tumor DLD cells showed the highest relative level of expression.

### Example 8

### Recombinant Expression of 21617 or 55562 in Bacterial Cells

In this example, 21617 or 55562 is expressed as a recombinant glutathione-S-transferase (GST) fusion polypeptide in E. coli and the fusion polypeptide is isolated and characterized. Specifically, 21617 or 55562 is fused to GST and this fusion polypeptide is expressed in E. coli, e.g., strain PEB199. Expression of the GST-21617 or 55562 fusion protein in PEB199 is induced with IPTG. The recombinant fusion polypeptide is purified from crude bacterial lysates of the induced PEB 199 strain by affinity chromatography on glutathione beads. Using polyacrylamide gel electrophoretic analysis of the polypeptide purified from the bacterial lysates, the molecular weight of the resultant fusion polypeptide is determined.

### Example 9

### Expression of Recombinant 21617 or 55562 Protein in COS Cells

To express the 21617 or 55562 gene in COS cells, the pcDNA/Amp vector by Invitrogen Corporation (San Diego, Calif.) is used. This vector contains an SV40 origin of replication, an ampicillin resistance gene, an E. coli replication origin, a CMV promoter followed by a polylinker region, and an SV40 intron and polyadenylation site. A DNA fragment encoding the entire 21617 or 55562 protein and an HA tag (Wilson et al. (1984) Cell 37:767) or a FLAG tag fused in-frame to its 3′ end of the fragment is cloned into the polylinker region of the vector, thereby placing the expression of the recombinant protein under the control of the CMV promoter.

To construct the plasmid, the 21617 or 55562 DNA sequence is amplified by PCR using two primers. The 5′ primer contains the restriction site of interest followed by approximately twenty nucleotides of the 21617 or 55562 coding sequence starting from the initiation codon; the 3′ end sequence contains complementary sequences to the other restriction site of interest, a translation stop codon, the HA tag or FLAG tag and the last 20 nucleotides of the 21617 or 55562 coding sequence. The PCR amplified fragment and the pcDNA/Amp vector are digested with the appropriate restriction enzymes and the vector is dephosphorylated using the CIAP enzyme (New England Biolabs, Beverly, Mass.). Preferably the two restriction sites chosen are different so that the 21617 or 55562 gene is inserted in the correct orientation. The ligation mixture is transformed into E. coli cells (strains HB101, DH5α, SURE, available from Stratagene Cloning Systems, La Jolla, Calif., can be used), the transformed culture is plated on ampicillin media plates, and resistant colonies are selected. Plasmid DNA is isolated from transformants and examined by restriction analysis for the presence of the correct fragment.

COS cells are subsequently transfected with the 21617 or 55562-pcDNA/Amp plasmid DNA using the calcium phosphate or calcium chloride co-precipitation methods, DEAE-dextran-mediated transfection, lipofection, or electroporation. Other suitable methods for transfecting host cells can be found in Sambrook, J., Fritsh, E. F., and Maniatis, T. (1989) Molecular Cloning: A Laboratory Manual. 2nd, ed., Cold Spring Harbor Laboratory, Cold Spring Harbor Laboratory Press, Cold Spring Harbor, N.Y. The expression of the 21617 or 55562 polypeptide is detected by radiolabelling (³⁵S-methionine or ³⁵S-cysteine available from NEN, Boston, Mass., can be used) and immunoprecipitation (Harlow, E. and Lane, D. (1988) Antibodies: A Laboratory Manual, Cold Spring Harbor Laboratory Press, Cold Spring Harbor, N.Y.) using an HA specific monoclonal antibody. Briefly, the cells are labeled for 8 hours with ³⁵S-methionine (or ³⁵S-cysteine). The culture media are then collected and the cells are lysed using detergents (RIPA buffer, 150 mM NaCl, 1% NP-40, 0.1% SDS, 0.5% DOC, 50 mM Tris, pH 7.5). Both the cell lysate and the culture media are precipitated with an HA specific monoclonal antibody. Precipitated polypeptides are then analyzed by SDS-PAGE.

Alternatively, DNA containing the 21617 or 55562 coding sequence is cloned directly into the polylinker of the pcDNA/Amp vector using the appropriate restriction sites. The resulting plasmid is transfected into COS cells in the manner described above, and the expression of the 21617 or 55562 polypeptide is detected by radiolabelling and immunoprecipitation using a 21617 or 55562 specific monoclonal antibody.

### EXAMPLES FOR 23566, 33489, AND 57779

### Example 10

### Identification and Characterization of Human 23566, 33489, or 57779 cDNA

The human 23566 nucleic acid sequence is recited as follows:

(SEQ ID NO:73)

TTATGGTTTTGTAGAATTTCTTTGACGAACCATTGGCAAAAAGCAGGAGC
AATCCATTCTCTGGCGTGAATTCCACAGTCCATCCAAATGATTTTCTTGG
GATTACCAGATGGTTGGCTGATCTTCAGATAATACATGGGGTGGGTCTCC
TAGGTCACTCCTAGGAAATGCTGTGTCACCACTTCCTTGTACTTCTCACT
GATCTCTCTCATCCACTCATAGATCTCTCCCATGGGGTGGTATATGTTAT
AGGAATACGTCTCCAGGCTCCATGGACTCACTGACTTGTCCACAATCTCT
TGTCTGTGTTGGGCTAAGGATCAGTTGGCTTTTTGAAAAAAGTGTTTTCT
CTTTCACTGCTTGCTCACCCTGGCCAACATGTGGTAGGCGCAGGGTCCTC
AGCCTTTCCGTGAAGACGATCAACGTTGCTCTTTCCCACGTGCTGATGTA
TTTTCTACCTGCCCGGGCGGCCGCTCGAGCGGCTACTCACTATAGGGCTC
GAGCGGCTACTCACTATAGGGCTCGAGCGGCCACTCACTATAGGGCTCGA
GCGGCTACTCACTATAGGGCTCGAGCGGCCGCCCGGGCAGGTATTCATTC
TCAAGGACACTTGATCCACTGCCAGAGAGGCCCAGAATTTTCTAACTTAC
TGTGTGGCAGAATGAAGCCTCTGCTTGAAACCCTTTATCTTTTGGGGATG
CTGGTTCCTGGAGGGCTGGGATATGATAGATCCTTAGCCCAACACAGACA
AGAGATTGTGGACAAGTCAGTGAGTCCATGGAGCCTGGAGACGTATTCCT
ATAACATATACCACCCCATGGGAGAGATCTATGAGTGGATGAGAGAGATC
AGTGAGAAGTACAAGGAAGTGGTGACACAGCATTTCCTAGGAGTGACCTA
TGAGACCCACCCCATATATTATCTGAAGATCAGCCAACCATCTGGTAATC
CCAAGAAAATCATTTGGATGGACTGTGGAATTCACGCCAGAGAATGGATT
GCTCCTGCTTTTTGCCAATGGTTCGTCAAAGAAATTCTACAAAACCATAA
AGACAACTCAAGGATACGCAAGCTCCTTAGGAACCTGGACTTCTATGTCC
TTCCAGTTCTTAACATAGATGGTTATATCTACACTTGGACAACTGATCGT
CTTTGGAGGAAATCCCGTTCACCCCATAATAATGGCACATGTTTTGGGAC
GGATCTCAATCGAAATTTCAATGCATCTTGGTGTAGTATTGGTGCCTCTA
GAAACTGCCAAGATCAAACATTCTGTGGGACAGGGCCAGTGTCTGAACCA
GAGACTAAAGCTGTTGCCAGCTTCATAGAGAGCAAGAAGGATGATATTTT
GTGCTTCCTGACCATGCACTCTTATGGGCAGTTAATTCTCACACCTTACG
GCTACACCAAAAATAAATCAAGTAACCACCCAGAAATGATTCAAGTTGGA
CAGAAGGCAGCAAATGCATTGAAAGCAAAGTATGGAACCAATTATAGAGT
TGGATCGAGTGCAGATATTTTATATGCCTCATCAGGGTCTTCAAGAGATT
GGGCCCGAGACATTGGGATTCCCTTCTCATATACGTTTGAGCTGAGGGAC
AGA.

The human 23566 sequence (SEQ ID NO:73) is approximately 1603 nucleotides long. The nucleic acid sequence includes an initiation codon (ATG) which is bolded and underscored above. The region between and inclusive of the initiation codon and the end of the sequence is a methionine-initiated coding sequence of about 1332 nucleotides (nucleotides indicated as “coding” of SEQ ID NO:73; SEQ ID NO:75). The coding sequence encodes a 444 amino acid protein (SEQ ID NO:74), which is recited as follows:

MDSLTCPQSLVCVGLRISWLFEKSVFSFTACSPWPTCGRRRVLSLSVKTINVALSHVLM YFLPARAAARAATHYRARAATHYRARAATHYRARAATHYRARAAARAGIHSQGHLI HCQRGPEFSNLLCGRMKPLLETLYLLGMLVPGGLGYDRSLAQHRQEIVDKSVSPWSLE TYSYNIYHPMGEIYEWMREISEKYKEVVTQHFLGVTYETHPIYYLKISQPSGNPKKIIW MDCGIHAREWIAPAFCQWFVKEILQNHKDNSRIRKLLRNLDFYVLPVLNIDGYIYTWTT DRLWRKSRSPHNNGTCFGTDLNRNFNASWCSIGASRNCQDQTFCGTGPVSEPETKAVA SFIESKKDDILCFLTMHSYGQLILTPYGYTKNKSSNHPEMIQVGQKAANALKAKYGTN YRVGSSADILYASSGSSRDWARDIGIPFSYTFELRDR (SEQ ID NO:74).

The human 33489 nucleic acid sequence is recited as follows:

(SEQ ID NO:76)

GCCGAGTGCTAGGCACCCGGGCTCTTCTGGGGGCTCCAGAGGCGCCGCCC
AAGAGACCCTGGGCCGGCGCCGGGCGCAGCTGCCTCTCCGTCTTTGTGTC
TGTCTCTGTGTCTGTCTGGCTATCTCCGAGTTTGCCTCCGCTTCCAGAAC
TAAGCCACCCAGACACCATCATCCCGAAAACCCCAGCCCTTCTCCCATGG
CAGGCTACTTGCCCCCCAAAGGCTACGCCCCTTCGCCCCCACCTCCCTAC
CCTGTCACCCCTGGGTACCCGGAGCCGGCGCTACATCCTGGGCCCGGGCA
GGCGCCAGTGCCCGCCCAGGTACCTGCCCCAGCTCCCGGCTTCGCCCTCT
TCCCCTCGCCTGGCCCCGTGGCCTTGGGGTCTGCTGCCCCCTTCTTGCCA
CTGCCAGGGGTGCCTTCTGGCCTCGAATTCCTGGTGCAGATTGATCAGAT
TTTGATTCACCAGAAGGCTGAGCGAGTGGAAACGTTCCTAGGCTGGGAGA
CCTGTAATCGGTATGAACTGCGCTCTGGGGCCGGGCAGCCCCTGGGTCAG
GCGGCCGAGGAGAGCAACTGCTGCGCCCGTCTGTGCTGTGGCGCCCGCCG
GCCGCTGCGTGTCCGCCTGGCCGACCCCGGGGACCGTGAGGTGCTGCGTT
TGCTCCGCCCGCTGCACTGTGGCTGCAGCTGCTGCCCCTGTGGCCTCCAG
GAGATGGAAGTACAGGCTCCACCAGGCACCACCATTGGCCACGTGCTACA
GACCTGGCATCCCTTCCTCCCCAAGTTCTCCATCCAGGATGCCGATCGCC
AGACAGTCTTGCGAGTGGTGGGGCCCTGCTGGACCTGTGGCTGTGGCACA
GACACCAACTTTGAGGTGAAGACTCGGGATGAATCCCGCAGTGTGGGCCG
CATCAGCAAGCAGTGGGGGGGCCTGGTCCGAGAAGCCCTCACAGATGCAG
ATGACTTTGGCCTACAGTTCCCGCTGGACCTGGATGTGAGGGTGAAGGCT
GTGCTGCTGGGAGCCACATTCCTCATTGACTACATGTTCTTTGAGAAGCG
AGGAGGCGCTGGGCCCTCTGCCATCACCAGTTAGAGGCCACCATGGTGTG
AGGAGACCATCACCTCGACCAGAACTCCAGATGGTCACCTGCCCTGGCCC
CTCCTCTGGGCAGCCCCTTTCCTCCATGTACACTGCAGGGGACAGAAGGG
GGGCCCCATCCCTACCCTACTCCCTGGCCGCCTGCCCCTGTGGTTCCCAA
GGAGGGGTATGTATGAGAGCCGCTCTCCTGCTACCTCCCACCACTGTCCC
AGCAGTCCCTCGGCACACAGGCATATCAGCTTTCACACTTTCCCCATGCA
CTCTCTCCCACCCCCTTCCAGGGCCTCTGCTCCAAAGGAGGCCTCTGGAA
CCCAGGACTCTGGGGTTTTACAAGAGGGCTGGGGTGTGGAAGGGCAAGCT
GCACCAAAGACGGTGGATATAGCCACCGCCCCCCCGCCGCTGCCTAGCAT
CTGCTTGGCCAATTAGTTCAGCCTCAGACCATGGCACTTTGAGGGGGTCT
CTACCTCCCCATCAACAGCTGCAGGGGGACCCCAGTGCCAACTTCCTCTC
CCACTAGGGCCCTGCCTTCAGCTGGTGCTTGCTGCGATTCCTGTGCCTTA
TGTAACTGCCCTTCCTTCCCTTGCCCTAGGAAAAAGGCTGCATCTTTATA
TGTTACATTCATATAAACTTTGTAACTTTTTGGAAAAAAAAAAAAAAA.

The human 33489 sequence (SEQ ID NO:76) is approximately 1748 nucleotides long. The nucleic acid sequence includes an initiation codon (ATG) and a termination codon (TAG) which are bolded and underscored above. The region between and inclusive of the initiation codon and the termination codon is a methionine-initiated coding sequence of about 888 nucleotides, including the termination codon (nucleotides indicated as “coding” of SEQ ID NO:76; SEQ ID NO:78). The coding sequence encodes a 295 amino acid protein (SEQ ID NO:77), which is recited as follows:

(SEQ ID NO:77)

MAGYLPPKGYAPSPPPPYPVTPGYPEPALHPGPGQAPVPAQVPAPAPGFA
LFPSPGPVALGSAAPFLPLPGVPSGLEFLVQIDQILIHQKAERVETFLGW
ETCNRYELRSGAGQPLGQAAEESNCCARLCCGARRPLRVRLADPGDREVL
RLLRPLHCGCSCCPCGLQEMEVQAPPGTTIGHVLQTWHPFLPKFSIQDAD
RQTVLRVVGPCWTCGCGTDTNFEVKTRDESRSVGRISKQWGGLVREALTD
ADDFGLQFPLDLDVRVKAVLLGATFLDYMFFEKRGGAGPSAITS.

The human 57779 nucleic acid sequence is recited as follows:

(SEQ ID NO:79)

GCACGAGCAGCCATGGAGTCGCTCCTGCTGCCGGTGCTGCTGCTGCTGGC
CATACTGTGGACGCAGGCTGCCGCCCTCATTAATCTCAAGTACTCGGTAG
AAGAGGAGCAGCGCGCCGGGACGGTGATTGCCAACGTGGCCAAAGACGCG
CGAGAGGCGGGCTTCGCGCTGGACCCCCGGCAGGCTTCAGCCTTTCGCGT
GGTGTCCAACTCGGCTCCACACCTAGTGGACATCAATCCCAGCTCTGGCC
TGCTGGTCACCAAGCAGAAGATTGACCGTGATCTGCTGTGCCGCCAGAGC
CCCAAGTGCATCATCTCGCTCGAGGTCATGTCCAGCTCAATGGAAATCTG
CGTGATAAAGGTGGAGATCAAGGACCTGAACGACAATGCGCCCAGTTTCC
CGGCAGCACGGATCGAGCTGGAGATCTCGGAGGCAGCCAGCCCTGGCACG
CGCATCCCGCTGGACAGCGCTTACGATCCAGACTCAGGAAGCTTTGGCGT
GCAGACTTACGAGCTCACGCCCAACGAGCTGTTCGGCCTGGAGATCAAGA
CGCGCGGCGACGGCTCCCGCTTTGCCGAACTCGTGGTGGAAAAGAGCCTG
GACCGCGAGACGCAGTCGCACTACAGCTTCCGAATCACTGCGCTAGACGG
TGGCGACCCGCCGCGCCTGGGCACCGTTGGCCTTAGTATCAAGGTGACCG
ACTCCAATGACAACAACCCGGTGTTTAGCGAGTCCACCTACGCGGTGAGC
GTGCCAGAAAACTCGCCTCCCAACACACCCGTCATCCGCCTCAACGCCAG
CGATCCAGACGAGGGCACCAACGGCCAGGTGGTCTACTCCTTCTATGGCT
ACGTCAACGACCGCACGCGCGAGCTCTTTCAGATCGACCCGCACAGTGGC
CTGGTCACTGTCACTGGCGCTTTAGACTACGAAGAGGGGCACGTGTACGA
ACTGGACGTGCAGGCTAAGGACTTGGGGCCCAATTCCATCCCGGCACACT
GCAAGGTCACCGTCAGCGTGCTGGACACCAATGACAATCCGCCGGTCATC
AACCTGCTGTCAGTCAACAGTGAGCTTGTGGAGGTCAGCGAGAGCGCCCC
CCCGGGCTACGTGATCGCCTTGGTGCGGGTGTCTGATCGCGACTCAGGCC
TCAATGGACGTGTGCAGTGCCGTTTGCTGGGCAATGTGCCCTTTCGACTG
CAGGAATATGAGAGCTTCTCCACTATTCTGGTGGACGGACGGCTGGACCG
CGAGCAGCACGACCAATACAACCTCACAATTCAGGCACGCGACGGCGGCG
TGCCCATGCTGCAGAGTGCCAAGTCCTTTACCGTGCTCATCACTGACGAA
AATGACAACCACCCGCACTTTTCCAAGCCCTACTACCAGGTCATTGTGCA
GGAGAACAACACGCCTGGCGCCTATCTGCTCTCTGTGTCTGCTCGCGACC
CCGACCTGGGTCTCAACGGCAGTGTCTCCTACCAGATCGTGCCGTCGCAG
GTGCGGGACATGCCTGTCTTCACCTATGTCTCCATCAATCCCAACTCAGG
CGACATCTACGCGCTGCGATCCTTTAACCACGAGCAGACCAAGGCGTTCG
AATTCAAGGTGCTGGCCAAGGACGGCGGCCTTCCCTCACTGCAAAGCAAC
GCTACGGTGCGGGTCATCATCCTCGACGTCAACGACAACACCCCGGTCAT
CACAGCCCCACCTCTGATTAACGGCACTGCCGAGGTCTACATACCCCGCA
ACTCTGGCATAGGCTACCTGGTGACTGTTGTCAAGGCAGAAGACTACGAT
GAGGGCGAAAATGGCCGAGTCACCTACGACATGACCGAGGGCGACCGCGG
CTTCTTTGAAATAGACCAGGTCAATGGCGAAGTCAGAACCACCCGCACCT
TCGGGGAGAGCTCCAAGTCCTCCTATGAGCTTATCGTGGTGGCTCACGAC
CACGGCAAGACATCTCTCTCTGCCTCTGCTCTCGTCCTAATCTACTTGTC
CCCTGCTCTCGATGCCCAAGAGTCAATGGGCTCTGTGAACTTGTCCTTGA
TTTTCATTATTGCCCTGGGCTCCATTGCGGGCATCCTCTTTGTAACTATG
ATCTTCGTGGCAATCAAGTGCAAGCGAGACAACAAAGAGATCCGGACCTA
CAACTGCAGAATTGCTGAGTACTCCTATGGGCATCAAAAGAAATCAAGCA
AGAAGAAAAAAATCAGTAAGAATGACAACCGCCTGGTACCCCGGGATGTG
GAGGAGACAGACAAGATGAACGTTGTCAGTTGCTCTTCCCTGACCTCCTC
CCTCAACTATTTTGACTACCACCAGCAGACGCTGCCCCTGGGCTGCCGCC
GCTCTGAGAGCACTTTCCTGAATGTGGAGAACCAGAATACCCGCAACACC
AGTGCTAACCACATCTACCATCACTCTTTCAACAGCCAGGGGCCCCAGCA
GCCTGACCTGATTATCAACGGTGTGCCTCTGCCTGAGAGCAAGGAAAGCC
AGCAGTGGTACTTTGTCGCTGCTGCAGCACTCTTTCCTCATGCCTGTTTT
GTGGGCTGCTGTCTGATATCCACTCCTACAGTGCTTTGGGAACAATATCA
AGCAGGGCATAAGGCTGTGGCCCAAAGAAAATGA.

The human 57779 sequence (SEQ ID NO:79), which is approximately 2634 nucleotides long. The nucleic acid sequence includes an initiation codon (ATG) and a termination codon (TGA) which are bolded and underscored above. The region between and inclusive of the initiation codon and the termination codon is a methionine-initiated coding sequence of about 2622 nucleotides, including the termination codon (nucleotides indicated as “coding” of SEQ ID NO:79; SEQ ID NO:81). The coding sequence encodes a 873 amino acid protein (SEQ ID NO:80), which is recited as follows:

(SEQ ID NO:80)

MESLLLPVLLLLAILWTQAAALINLKYSVEEEQRAGTVTANVAKDAREAG
FALDPRQASAFRVVSNSAPHLVDINPSSGLLVTKQKIDRDLLCRQSPKCI
ISLEVMSSSMEICVIKVEIKDLNDNAPSFPAARIELEISEAASPGTRIIP
LDSAYDPDSGSFGVQTYELTPNELFGLEIKTRGDGSRFAELVVEKSLDRE
TQSHYSFRITALDGGDPPRLGTVGLSIKVTDSNDNNPVFSESTYAVSVPE
NSPPNTPVIRLNASDPDEGTNGQVVYSFYGYVNPRTRELFQIDPHSGLVT
VTGALDYEEGHVYELDVQAKDLGPNSIPAHCKVTVSVLDTNDNPPVINLL
SVNSELVEVSESAPPGYVIALVRVSDRDSGLNGRVQCRILGNVPFRLQEY
ESFSTILVDGRLDREQHDQYNLTIQARDGGVPMLQSAKSFTVLITDENDN
HPHFSKPYYQVIVQENNTPGAYLLSVSARDPDLGLNGSVSYQIVPSQVRD
MPVFTYVSTNPNSGDIYALRSFNLTEQTKAFEFKVLAKDGGLPSLQSNAT
VRVIILDVDNTPVITAYPLNGTAEVYIIRNSGIGYLVTVVKAEDYDEGEN
GRVTYDMTEGDRGFFFIDQVNGEVRTTRTFGESSKSSYELIVVAHDHGKT
SLSASALVLIYLSPALDAQESMGSVNLSLTFITALGSIAGILFVTMIFVA
IKCKRDNKEIRTYNCRIAEYSYGHQKKSSKKKIKISKNDNRLVPRDVEET
DKMNVVSCSSLTSSLNYFDYHQQTLPLGCRRSESTFLNVENQNTRNTSAN
HIYLIHSFNSQGPQQPDLIINGVPLPESKESQQWYFVAAAALFPHACFVG
CCLISTPTVLWEQYQAGHKAVAQRK.

### Example 11

### Tissue Distribution of 23566, 33489, or 57779 mRNA by TaqMan Analysis

Endogenous human 23566, 33489, or 57779 gene expression was determined using the Perkin-Elmer/ABI 7700 Sequence Detection System which employs TaqMan technology. Briefly, TaqMan technology relies on standard RT-PCR with the addition of a third gene-specific oligonucleotide (referred to as a probe) which has a fluorescent dye coupled to its 5′ end (typically 6-FAM) and a quenching dye at the 3′ end (typically TAMRA). When the fluorescently tagged oligonucleotide is intact, the fluorescent signal from the 5′ dye is quenched. As PCR proceeds, the 5′ to 3′ nucleolytic activity of Taq polymerase digests the labeled primer, producing a free nucleotide labeled with 6-FAM, which is now detected as a fluorescent signal. The PCR cycle where fluorescence is first released and detected is directly proportional to the starting amount of the gene of interest in the test sample, thus providing a quantitative measure of the initial template concentration. Samples can be internally controlled by the addition of a second set of primers/probe specific for a housekeeping gene such as GAPDH which has been labeled with a different fluorophore on the 5′ end (typically VIC).

To determine the level of 23566 mRNA in various human tissues a primer/probe set was designed. Total RNA was prepared from a series of human tissues using an RNeasy kit from Qiagen. First strand cDNA was prepared from 1 μg total RNA using an oligo-dT primer and Superscript II reverse transcriptase (Gibco/BRL). cDNA obtained from approximately 50 ng total RNA was used per TaqMan reaction. Tissues tested include the human tissues and cell lines shown in Tables 1-3. Elevated levels of 23566 mRNA were detected in, e.g., teste, neural tissues (e.g., brain cortex, hypothalamus, dorsal root ganglia, and spinal chord), kidney, adrenal gland, muscle (e.g., skeletal muscle), pancreas, pituitary gland, aorta, and lymph nodes.

Table 9
Expression of human 23566 mRNA

| | Relative |
|---|---|
| Tissue | Expression |
| Adrenal Gland | 3.34 |
| Brain | 0.48 |
| Heart | 0.24 |
| Kidney | 3.49 |
| Liver | 0.43 |
| Lung | 0.26 |
| Mammary Gland | 0.91 |
| Pancreas | 0.13 |
| Placenta | 0.09 |
| Prostate | 0.67 |
| Pituatary Gland | 0.74 |
| Muscle | 0.30 |
| Sm. Intestine | 0.50 |
| Spleen | 0.17 |
| Stomach | 0.50 |
| Teste | 2.67 |
| Thymus | 0.13 |
| Trachea | 0.16 |
| Uterus | 0.61 |
| Spinal Cord | 0.66 |
| Skin | 1.27 |
| DRG | 1.04 |

Expression of human 23566 mRNA, as shown in Table 9, was detected in a wide range of human tissues, including teste, kidney, adrenal gland, brain, dorsal root ganglia, spinal chord, muscle, pancreas, and pituitary gland, as well as additional tissues that displayed lower expression.

Table 10
Expression of human 23566 mRNA

| | Relative |
|---|---|
| Tissue | Expression |
| Aorta/normal | 0.15775791 |
| Fetal heart/normal | 0.00133475 |
| Heart normal | 0.00153322 |
| Heart/CHF | 0.04514732 |
| Vein/Normal | 0.0007303 |
| Spinal cord/Normal | 0.00823386 |
| Brain cortex/Normal | 0.64877237 |
| Brain hypothalamus/Normal | 0.23994644 |
| Glial cells (Astrocytes) | 0.0062835 |
| Brain/Glioblastoma | 0.00108792 |
| Breast/Normal | 0.00257857 |
| Breast tumor/IDC | 0.00030919 |
| OVARY/Normal | 0.00556572 |
| OVARY/Tumor | 0.0145361 |
| Prostate/Normal | 0.00885547 |
| Prostate/Tumor | 0.01136534 |
| Colon/normal | 0.00068376 |
| Colon/tumor | 0.00256964 |
| Colon/IBD | 0.00029866 |
| Kidney/normal | 0.13497689 |
| Liver/normal | 0.00121974 |
| Liver fibrosis | 0.0038147 |
| Fetal Liver/normal | 0.01310067 |
| Lung/normal | 0.000224 |
| Lung/tumor | 0.00061199 |
| Lung/COPD | 0.00067435 |
| Spleen/normal | 0.00359644 |
| Tonsil/normal | 0.00015675 |
| Lymph node/normal | 0.08308802 |
| Thymus/normal | 0.00340244 |
| Epithelial Cells (prostate) | 0.02120847 |
| Endothelial Cells (aortic) | 0.00038464 |
| Skeletal Muscle/Normal | 0.14516688 |
| Fibroblasts (Dermal) | 0.00019366 |
| Skin/normal | 0.00382794 |
| Adipose/Normal | 0.00073538 |
| Osteoblasts (primary) | 0.00051284 |
| Osteoblasts (Undiff) | 0.00012214 |
| Osteoblasts(Diff) | 0.00018006 |
| Osteoclasts | 2.0006E − 05 |
| Aortic SMC Early | 0.000448 |
| Aortic SMC Late | 0.00436678 |
| shear HUVEC | 0.00160388 |
| static HUVEC | 0.00070055 |
| Osteoclasts(Undiff) | 1.4245E − 05 |

Expression of human 23566 mRNA, as shown in Table 10, was highest in normal brain cortex and hypothalamus samples. Relatively moderate expression was found in normal aorta, kidney, lymph node and skeletal muscle, and low levels of expression were detected in a variety of other tissues, e.g., spinal chord, ovary, prostate, fetal liver, and epithelial cells. In addition, elevated expression was detected in heart tissue from a patient that suffered from congestive heart failure.

Table 11
Expression of human 23566 mRNA

| | Relative |
|---|---|
| Tissue | Expression |
| MK cortex | 0.0587521 |
| MK DRG | 0.00323008 |
| MK spinal cord | 0.01109293 |
| MK sciatic nerve | 0.0081205 |
| MK kidney | 0.00413122 |
| MK hairy skin | 0.00972413 |
| MK heart LV | 0.00689987 |
| MK gastro muscle | 0.016241 |
| MK liver | 0.06679039 |
| Hu. Brain | 9.75258241 |
| Hu. Spinal cord | 2.7430564 |
| Hu. Heart | 0.3133364 |
| Hu. Kidney | 3.55730404 |
| Hu. Liver | 0.76090291 |
| Hu. Lung | 0.18247672 |

Expression of human 23566 mRNA, as shown in Table 11, was detected in several tissues, with highest expression observed in normal brain tissue (e.g., brain cortex and hypothalamus). Moderate to high expression was found in spinal cord and kidney tissue, moderate levels were found in heart and liver tissue, and a lower level of expression was observed in lung tissue.

To determine the level of 33489 mRNA in various human tissues a primer/probe set was designed. Total RNA was prepared from a series of human tissues using an RNeasy kit from Qiagen. First strand cDNA was prepared from 1 μg total RNA using an oligo-dT primer and Superscript II reverse transcriptase (Gibco/BRL). cDNA obtained from approximately 50 ng total RNA was used per TaqMan reaction. Tissues tested include the human tissues and several cell lines shown in Table 12. Elevated levels of human 33489 mRNA were detected in, e.g., coronary smooth muscle cells and human umbilical vein endothelial cells.

Table 12
Expression of human 33489 mRNA

| | Relative |
|---|---|
| Tissue | Expression |
| Artery normal | 17.277 |
| Aorta diseased | 9.6183 |
| Vein normal | 6.1936 |
| Coronary Smooth Muscle Cells | 69.8304 |
| Human Umbilical Vein Endothelial | |
| Cells (HUVEC) | 64.2571 |
| Hemangioma | 18.453 |
| Heart normal | 10.4888 |
| Heart congestive heart failure | 10.202 |
| Kidney | 12.4734 |
| Skeletal Muscle | 13.5084 |
| Adipose normal | 6.8961 |
| Pancreas | 13.7445 |
| primary osteoblasts | 8.7591 |
| Osteoclasts (differentiated) | 0.9936 |
| Skin normal | 15.5171 |
| Spinal cord normal | 4.3948 |
| Brain Cortex normal | 13.7445 |
| Brain Hypothalamus normal | 12.5602 |
| Nerve | 15.6792 |
| Dorsal Root Ganglion | 13.5084 |
| Breast normal | 8.4901 |
| Breast tumor | 3.4124 |
| Ovary normal | 11.6381 |
| Ovary Tumor | 6.5695 |
| Prostate Normal | 8.9432 |
| Prostate Tumor | 7.34 |
| Salivary glands | 1.2533 |
| Colon normal | 2.2436 |
| Colon Tumor | 8.9122 |
| Lung normal | 4.0161 |
| Lung tumor | 13.5084 |
| Lung Chronic Obstructive | |
| Pulmonary Disease | 12.1323 |
| Colon Inflammatory Bowel Disease | 4.0022 |
| Liver normal | 2.4722 |
| Liver fibrosis | 5.3361 |
| Spleen normal | 3.4962 |
| Tonsil normal | 3.7471 |
| Lymph node normal | 6.7542 |
| Small intestine normal | 2.5951 |
| Macrophages | 0.37 |
| Synovium | 3.7342 |
| Bone marrow mononuclear cells | 0.8269 |
| Activated peripheral blood | |
| mononuclear cells | 3.7994 |
| Neutrophils | 0.7742 |
| Megakaryocytes | 3.1509 |
| Erythroid | 5.7589 |
| positive control | 15.0405 |

Expression of human 33489 mRNA, as shown in Table 12, was detected in a wide range of tissues, with highest expression observed in smooth muscle cells (e.g., coronary smooth muscle cells) and endothelial cells (e.g., HUVECs). Moderate expression of human 33489 mRNA was detected in, e.g., arteries, veins, heart, kidney, skeletal muscle, adipose tissue, pancreas, osteoblasts, skin, neural tissue (e.g., brain cortex, hypothalamus, dorsal root ganglia), breast, ovary, prostate, colon, lung, liver, lymph nodes, and blood cells (e.g., erythroid cells). Furthermore, elevated expression of human 33489 mRNA was detected in colon and lung tumors, as compared to normal colon and lung tissue, while reduced expression was detected in breast and ovary tumors, as compared to normal breast and ovary tissue. Elevated expression of human 33489 mRNA was also detected in COPD (chronic obstructive pulmonary disease) lung tissue, as compared to normal lung tissue.

To determine the level of 57779 mRNA in various human tissues a primer/probe set was designed. Total RNA was prepared from a series of human tissues using an RNeasy kit from Qiagen. First strand cDNA was prepared from 1 μg total RNA using an oligo-dT primer and Superscript II reverse transcriptase (Gibco/BRL). cDNA obtained from approximately 50 ng total RNA was used per TaqMan reaction. Tissues tested include the human tissues and several cell lines shown in Table 13. Abundant human 57779 mRNA was detected in the brain cortex and in brain hypothalamus.

Table 13
Expression of human 57779 mRNA

| | Relative |
|---|---|
| Tissue | Expression |
| Artery normal | 0.92 |
| Aorta diseased | 0.47 |
| Vein normal | 0.00 |
| Coronary smooth muscle cells | 0.00 |
| HUVECs (endothelial cells) | 1.97 |
| Hemangioma | 0.58 |
| Heart normal | 0.16 |
| Heart CHF | 0.30 |
| Kidney | 0.09 |
| Skeletal Muscle | 1.12 |
| Adipose normal | 1.02 |
| Pancreas | 0.00 |
| primary osteoblasts | 0.00 |
| Osteoclasts (diff) | 0.00 |
| Skin normal | 0.85 |
| Spinal cord normal | 0.77 |
| Brain Cortex normal | 54.60 |
| Brain Hypothalamus normal | 17.46 |
| Nerve | 1.12 |
| DRG (Dorsal Root Ganglion) | 2.31 |
| Breast normal | 2.98 |
| Breast tumor | 0.00 |
| Ovary normal | 5.15 |
| Ovary Tumor | 1.03 |
| Prostate Normal | 0.09 |
| Prostate Tumor | 0.21 |
| Salivary glands | 0.18 |
| Colon normal | 0.07 |
| Colon Tumor | 0.78 |
| Lung normal | 0.01 |
| Lung tumor | 2.92 |
| Lung COPD | 0.04 |
| Colon IBD | 0.06 |
| Liver normal | 0.04 |
| Liver fibrosis | 0.58 |
| Spleen normal | 0.00 |
| Tonsil normal | 0.01 |
| Lymph node normal | 0.03 |
| Small intestine normal | 0.07 |
| Skin-Decubitus | 0.65 |
| Synovium | 0.00 |
| BM-MNC | 0.00 |
| Activated PBMC | 0.00 |
| Neutrophils | 0.00 |
| Megakaryocytes | 0.00 |
| Erythroid | 0.00 |

Expression of human 57779 mRNA, as shown in Table 13, was detected in numerous tissues, with highest expression levels detected in neural tissue (e.g., the brain cortex and hypothalamus). Moderate to low 57779 mRNA expression was observed in, e.g., ovary, breast, lung, dorsal root ganglia, endothelial cells (e.g., HUVECs), arteries, skeletal muscle, adipose, skin, heart, kidney, prostate, salivary gland, colon, liver, tonsil, lymph node, and small intestine tissues. Furthermore, elevated levels of 57779 mRNA were detected in colon and lung tumors, as compared to normal colon and lung tissues, while reduced levels of expression were detected in breast and ovary tumors, as compared to normal breast and ovary tissues. Elevated levels of 57779 mRNA expression were also detected in fibrotic liver tissue as compared to normal liver tissue.

### Example 12

### Tissue Distribution of 23566, 33489, or 57779 mRNA by Northern Analysis

Northern blot hybridizations with various RNA samples can be performed under standard conditions and washed under stringent conditions, i.e., 0.2× SSC at 65° C. A DNA probe corresponding to all or a portion of the 23566, 33489, or 57779 cDNA (SEQ ID NO:73, SEQ ID NO:76, OR SEQ ID NO:79) can be used. The DNA was radioactively labeled with ³²P-dCTP using the Prime-It Kit (Stratagene, La Jolla, Calif.) according to the instructions of the supplier. Filters containing mRNA from mouse hematopoietic and endocrine tissues, and cancer cell lines (Clontech, Palo Alto, Calif.) can be probed in ExpressHyb hybridization solution (Clontech) and washed at high stringency according to manufacturer's recommendations.

### Example 13

### Recombinant Expression of 23566, 33489, or 57779 in Bacterial Cells

In this example, 23566, 33489, or 57779 is expressed as a recombinant glutathione-S-transferase (GST) fusion polypeptide in E. coli and the fusion polypeptide is isolated and characterized. Specifically, 23566, 33489, or 57779 is fused to GST and this fusion polypeptide is expressed in E. coli, e.g., strain PEB199. Expression of the GST-23566, 33489, or 57779 fusion protein in PEB199 is induced with IPTG. The recombinant fusion polypeptide is purified from crude bacterial lysates of the induced PEB199 strain by affinity chromatography on glutathione beads. Using polyacrylamide gel electrophoretic analysis of the polypeptide purified from the bacterial lysates, the molecular weight of the resultant fusion polypeptide is determined.

### Example 14

### Expression of Recombinant 23566, 33489, or 57779 Protein in COS Cells

To express the 23566, 33489, or 57779 gene in COS cells (e.g., COS-7 cells, CV-1 origin SV40 cells; Gluzman (1981) CellI23:175-182), the pcDNA/Amp vector by Invitrogen Corporation (San Diego, Calif.) is used. This vector contains an SV40 origin of replication, an ampicillin resistance gene, an E. coli replication origin, a CMV promoter followed by a polylinker region, and an SV40 intron and polyadenylation site. A DNA fragment encoding the entire 23566, 33489, or 57779 protein and an HA tag (Wilson et al. (1984) Cell 37:767) or a FLAG tag fused in-frame to its 3′ end of the fragment is cloned into the polylinker region of the vector, thereby placing the expression of the recombinant protein under the control of the CMV promoter.

To construct the plasmid, the 23566, 33489, or 57779 DNA sequence is amplified by PCR using two primers. The 5′ primer contains the restriction site of interest followed by approximately twenty nucleotides of the 23566, 33489, or 57779 coding sequence starting from the initiation codon; the 3′ end sequence contains complementary sequences to the other restriction site of interest, a translation stop codon, the HA tag or FLAG tag and the last 20 nucleotides of the 23566, 33489, or 57779 coding sequence. The PCR amplified fragment and the pcDNA/Amp vector are digested with the appropriate restriction enzymes and the vector is dephosphorylated using the CIAP enzyme (New England Biolabs, Beverly, Mass.). Preferably the two restriction sites chosen are different so that the 23566, 33489, or 57779 gene is inserted in the correct orientation. The ligation mixture is transformed into E. coli cells (strains HB101, DH5α, SURE, available from Stratagene Cloning Systems, La Jolla, Calif., can be used), the transformed culture is plated on ampicillin media plates, and resistant colonies are selected. Plasmid DNA is isolated from transformants and examined by restriction analysis for the presence of the correct fragment.

COS cells are subsequently transfected with the 23566, 33489, or 57779-pcDNA/Amp plasmid DNA using the calcium phosphate or calcium chloride co-precipitation methods, DEAE-dextran-mediated transfection, lipofection, or electroporation. Other suitable methods for transfecting host cells can be found in Sambrook, J., Fritsh, E. F., and Maniatis, T. (1989) Molecular Cloning: A Laboratory Manual. 2nd, ed., Cold Spring Harbor Laboratory, Cold Spring Harbor Laboratory Press, Cold Spring Harbor, N.Y. The expression of the 23566, 33489, or 57779 polypeptide is detected by radiolabelling (³⁵S-methionine or ³⁵S-cysteine available from NEN, Boston, Mass., can be used) and immunoprecipitation (Harlow, E. and Lane, D. (1988) Antibodies: A Laboratory Manual, Cold Spring Harbor Laboratory Press, Cold Spring Harbor, N.Y.) using an HA specific monoclonal antibody. Briefly, the cells are labeled for 8 hours with ³⁵S-methionine (or ³⁵S-cysteine). The culture media are then collected and the cells are lysed using detergents (RIPA buffer, 150 mM NaCl, 1% NP-40, 0.1% SDS, 0.5% DOC, 50 mM Tris, pH 7.5). Both the cell lysate and the culture media are precipitated with an HA specific monoclonal antibody. Precipitated polypeptides are then analyzed by SDS-PAGE.

Alternatively, DNA containing the 23566, 33489, or 57779 coding sequence is cloned directly into the polylinker of the pcDNA/Amp vector using the appropriate restriction sites. The resulting plasmid is transfected into COS cells in the manner described above, and the expression of the 23566, 33489, or 57779 polypeptide is detected by radiolabelling and immunoprecipitation using a 23566, 33489, or 57779 specific monoclonal antibody.

Equivalents

Those skilled in the art will recognize, or be able to ascertain using no more than routine experimentation, many equivalents to the specific embodiments of the invention described herein. Such equivalents are intended to be encompassed by the following claims.

What is claimed is:

1. An isolated nucleic acid molecule selected from the group consisting of:
a) a nucleic acid comprising the nucleotide sequence of SEQ ID NO:1, 3, 4, 6, 7, 9, 10, 12, 13, 15, 16, 18, 19, 21, 22, 24, 25, 27, 28, 30, 63, 65, 66, 68, 73, 75, 76, 78, 79, or 81; and
b) a nucleic acid molecule which encodes a polypeptide comprising the amino acid sequence of SEQ ID NO:2, 5, 8, 11, 14, 17, 20, 23, 26, 29, 64, 67, 74, 77, or 80.

2. The nucleic acid molecule of claim 1, further comprising vector nucleic acid sequences.

3. The nucleic acid molecule of claim 1, further comprising nucleic acid sequences encoding a heterologous polypeptide.

4. A host cell which contains the nucleic acid molecule of claim 1.

5. An isolated polypeptide comprising the amino acid sequence of SEQ ID NO:2, 5, 8, 11, 14, 17, 20, 23, 26, 29, 64, 67, 74, 77, or 80.

6. The polypeptide of claim 5 further comprising heterologous amino acid sequences.

7. An antibody or antigen-binding fragment thereof that selectively binds to a polypeptide of claim 5.

8. A method for producing a polypeptide comprising the amino acid sequence of SEQ ID NO:2, 5, 8, 11, 14, 17, 20, 23, 26, 29, 64, 67, 74, 77, or 80, the method comprising culturing the host cell of claim 4 under conditions in which the nucleic acid molecule is expressed.

9. A method for detecting the presence of a polypeptide of claim 5 in a sample, comprising:
a) contacting the sample with a compound which selectively binds to the polypeptide; and
b) determining whether the compound binds to the polypeptide in the sample.

10. The method of claim 9, wherein the compound which binds to the polypeptide is an antibody.

11. A kit comprising a compound which selectively binds to a polypeptide of claim 5 and instructions for use.

12. A method for detecting the presence of a nucleic acid molecule of claim 1 in a sample, comprising the steps of:
a) contacting the sample with a nucleic acid probe or primer which selectively hybridizes to the nucleic acid molecule; and
b) determining whether the nucleic acid probe or primer binds to a nucleic acid molecule in the sample.

13. The method of claim 12, wherein the sample comprises mRNA molecules and is contacted with a nucleic acid probe.

14. A kit comprising a compound which selectively hybridizes to a nucleic acid molecule of claim 1 and instructions for use.

15. A method for identifying a compound which binds to a polypeptide of claim 5 comprising the steps of:
a) contacting a polypeptide, or a cell expressing a polypeptide of claim 5 with a test compound; and
b) determining whether the polypeptide binds to the test compound.

16. A method for modulating the activity of a polypeptide of claim 5, comprising contacting a polypeptide or a cell expressing a polypeptide of claim 5 with a compound which binds to the polypeptide in a sufficient concentration to modulate the activity of the polypeptide.

17. A method of inhibiting aberrant activity of a 47476, 67210, 49875, 46842, 33201, 83378, 84233, 64708, 85041, 84234, 21617, 55562, 23566, 33489, or 57779-expressing cell, comprising contacting a 47476, 67210, 49875, 46842, 33201, 83378, 84233, 64708, 85041, 84234, 21617, 55562, 23566, 33489, or 57779-expressing cell with a compound that modulates the activity or expression of a polypeptide of claim 5, in an amount which is effective to reduce or inhibit the aberrant activity of the cell.

18. The method of claim 17, wherein the compound is selected from the group consisting of a peptide, a phosphopeptide, a small organic molecule, and an antibody.

19. A method of treating or preventing a disorder characterized by aberrant activity of a 47476, 67210, 49875, 46842, 33201, 83378, 84233, 64708, 85041, 84234, 21617, 55562, 23566, 33489, or 57779-expressing cell, in a subject, comprising:
administering to the subject an effective amount of a compound that modulates the activity or expression of a nucleic acid molecule of claim 1, such that the aberrant activity of the 47476, 67210, 49875, 46842, 33201, 83378, 84233, 64708, 85041, 84234, 21617, 55562, 23566, 33489, or 57779-expressing cell is reduced or inhibited.


SEQUENCE LISTING (86 sequences)

SEQ ID NO: 1 - DNA, 3134 bp, Homo sapiens
  CDS (113)...(2131)
caccgaggaggccccagctccctaggggctgagaagctggagtcctgggcaaggggaggagctgagccctactcttgcaagacccccggcctcctcaccccacgcgggaagcatgaacagaaaagacagtaagaggaagtcccaccaggaatgcaccggaaaaataggagggcgaggccggccccgccaagtgcgccgccacaagacatgccccagccctcgggaaatcagcaaggtcatggcttccatgaacctgggcctgctgagtgagggcggctgcagcgaagatgagctgctggagaaatgcatccagtccttcgattcagctggcagcctgtgccacgaggaccacatgctcaacatggtgctggccatgcacagctgggtgctgccgtccgccgacctggctgcccgcctgctgacctcataccagaaggccacaggggacacccaggagctgagacggctgcagatctgtcacctggtcaggtactggctgatgcgacaccctgaggtgatgcaccaggatccccagctagaagaagtcataggtcgtttctgggccaccgtggcccgggagggcaactcagcccagagaagactgggagactcttctgacctcctgagccctggtggccctggccccccactcccaatgagcagcccaggcctgggcaaaaagcgcaaagtgtccttgcttttcgaccacttggagacgggggagctggctcagcacctcacctacctggagttccggtccttccaggctatcacgccccaggacctgcggagctacgttttgcagggctcagtacgaggctgcccggccctggagggctccgtaggtctcagcaacagcgtgtcccgctgggtgcaggtgatggtgctgagccgtcccgggcccctacagcgtgcacaggtgctggacaagttcattcacgtggcacagaggctccaccagctgcagaatttcaacacgctgatggcagtcacagggggcctgtgtcacagtgccatctccagactcaaggactcccatgcccacctgagccctgacagcaccaaggccctcctggagctcactgagctccttgcctcccacaacaactacgcccgctaccgccgcacctgggctggctgcgcgggtttccggctgcctgtactgggcgtgcacctcaaggacctggtgtccctgcatgaggcacagcccgacaggttgcctgacggccgcctgcacctacccaagctgaacaacctctacctgcggctgcaggagctggtggccctccaagggcagcatccaccctgcagcgccaatgaggatctgctgcacctgctcacgctctccctggacctcttctacacggaagacgagatctatgagctttcttatgcccgggagccgcgttgtcccaagagcctgccaccctcccccttcaatgcacctctggtggtggagtgggcccctggtgtgacacccaagccggacagggtcacactgggtcggcatgtggagcagctggtggagtctgtgttcaagaattatgaccctgaaggccgaggaacaatctctcaggaggactttgagcgactctcgggcaattttcccttcgcctgccatgggcttcacccacccccacgccaggggagaggatccttcagcagagaggagctgacagggtacctgctccgggccagcgccatctgctccaagttgggcctggccttcctgcacaccttccatgaggtcaccttccgaaagcctaccttctgcgacagctgcagtggcttcctctggggtgtcaccaagcaaggctaccgctgtcgggagtgcgggctgtgttgccacaaacactgcagagaccaggtgaaggtagaatgtaagaagaggccaggggccaagggcgatgcaggaccccccggagctcctgtcccatccacaccagctccccatgccagctgtggctccgaggaaaatcactcctacacgctatccctggagcctgagactgggtgccagcttcgccatgcctggacccagactgaatccccacacccttcctgggaaacagatacggtcccctgcccggtgatggacccaccatcaactgcatcctccaagctggattcctagacatctcttggtctcctctcttcctcactcccttcccccagtcagtcctgagtcctgccgtcagactctggcagggctcccagaggtaggcctccagaggcatccgccttcccatccacactgcctttagtggtacgtccgtcatctttttccctggaagtgactttcctcttttgcatcctggtggatgctaatcctgctccctttccctgcacttagtatctgtgcggctgtgtgttggatacatttagactcaggcctcattctaccaactccctacatctccatttctttttttttttgagacagagtcttgctctgtcacccaggctggagtgcaatgatgcaatctcagctcactgcaacttccgcctcctgggttcaagtgattcttctgcctcagcctcccaagtagctgggattacaggcacgcgccaccatgcccggctaatttttgtatttttagtagagacggggtttccccatgttggccaagatggtctcaaactcctgacctcatgatccaccagcctcagcctcccaaagtgctgggaatacaggcgtgagccaccacgcccagctcctgtttttctgcatagaatgtgctccctctgacagcctatactttttccctctttatctcgttgaacgtctgtctttcctcaacaggagcatcaactccaggagaacagggatttctatcaatgtcattcactgctgcatccccagtgcctagaacagggctggcaggtggtaggcgcctgacagatgcttgtctgatgcctgaatgtctccttacccatgcccacggcacaggatagatgtgctatagggcaaagaactttgagggtcgagcagcaggggcccattcagtcccagggagcagagacccccccaaccccttcacaaaccccaacacccctgacttggcccccacagagagaggtcctacagctgtcataaattaaatttattctctggaaaaaaaaaaaaaaaaaaa

SEQ ID NO: 2 - protein, 673 aa, Homo sapiens
MNRKDSKRKSHQECTGKIGGRGRPRQVRRHKTCPSPREISKVMASMNLGLLSEGGCSEDELLEKCIQSFDSAGSLCHEDHMLNMVLAMHSWVLPSADLAARLLTSYQKATGDTQELRRLQICHLVRYWLMRHPEVMHQDPQLEEVIGRFWATVAREGNSAQRRLGDSSDLLSPGGPGPPLPMSSPGLGKKRKVSLLFDHLETGELAQHLTYLEFRSFQAITPQDLRSYVLQGSVRGCPALEGSVGLSNSVSRWVQVMVLSRPGPLQRAQVLDKFIHVAQRLHQLQNFNTLMAVTGGLCHSAISRLKDSHAHLSPDSTKALLELTELLASHNNYARYRRTWAGCAGFRLPVLGVHLKDLVSLHEAQPDRLPDGRLHLPKLNNLYLRLQELVALQGQHPPCSANEDLLHLLTLSLDLFYTEDEIYELSYAREPRCPKSLPPSPFNAPLVVEWAPGVTPKPDRVTLGRHVEQLVESVFKNYDPEGRGTISQEDFERLSGNFPFACHGLHPPPRQGRGSFSREELTGYLLRASAICSKLGLAFLHTFHEVTFRKPTFCDSCSGFLWGVTKQGYRCRECGLCCHKHCRDQVKVECKKRPGAKGDAGPPGAPVPSTPAPHASCGSEENHSYTLSLEPETGCQLRHAWTQTESPHPSWETDTVPCPVMDPPSTASSKLDS

SEQ ID NO: 3 - DNA, 2022 bp, Homo sapiens
atgaacagaaaagacagtaagaggaagtcccaccaggaatgcaccggaaaaataggagggcgaggccggccccgccaagtgcgccgccacaagacatgccccagccctcgggaaatcagcaaggtcatggcttccatgaacctgggcctgctgagtgagggcggctgcagcgaagatgagctgctggagaaatgcatccagtccttcgattcagctggcagcctgtgccacgaggaccacatgctcaacatggtgctggccatgcacagctgggtgctgccgtccgccgacctggctgcccgcctgctgacctcataccagaaggccacaggggacacccaggagctgagacggctgcagatctgtcacctggtcaggtactggctgatgcgacaccctgaggtgatgcaccaggatccccagctagaagaagtcataggtcgtttctgggccaccgtggcccgggagggcaactcagcccagagaagactgggagactcttctgacctcctgagccctggtggccctggccccccactcccaatgagcagcccaggcctgggcaaaaagcgcaaagtgtccttgcttttcgaccacttggagacgggggagctggctcagcacctcacctacctggagttccggtccttccaggctatcacgccccaggacctgcggagctacgttttgcagggctcagtacgaggctgcccggccctggagggctccgtaggtctcagcaacagcgtgtcccgctgggtgcaggtgatggtgctgagccgtcccgggcccctacagcgtgcacaggtgctggacaagttcattcacgtggcacagaggctccaccagctgcagaatttcaacacgctgatggcagtcacagggggcctgtgtcacagtgccatctccagactcaaggactcccatgcccacctgagccctgacagcaccaaggccctcctggagctcactgagctccttgcctcccacaacaactacgcccgctaccgccgcacctgggctggctgcgcgggtttccggctgcctgtactgggcgtgcacctcaaggacctggtgtccctgcatgaggcacagcccgacaggttgcctgacggccgcctgcacctacccaagctgaacaacctctacctgcggctgcaggagctggtggccctccaagggcagcatccaccctgcagcgccaatgaggatctgctgcacctgctcacgctctccctggacctcttctacacggaagacgagatctatgagctttcttatgcccgggagccgcgttgtcccaagagcctgccaccctcccccttcaatgcacctctggtggtggagtgggcccctggtgtgacacccaagccggacagggtcacactgggtcggcatgtggagcagctggtggagtctgtgttcaagaattatgaccctgaaggccgaggaacaatctctcaggaggactttgagcgactctcgggcaattttcccttcgcctgccatgggcttcacccacccccacgccaggggagaggatccttcagcagagaggagctgacagggtacctgctccgggccagcgccatctgctccaagttgggcctggccttcctgcacaccttccatgaggtcaccttccgaaagcctaccttctgcgacagctgcagtggcttcctctggggtgtcaccaagcaaggctaccgctgtcgggagtgcgggctgtgttgccacaaacactgcagagaccaggtgaaggtagaatgtaagaagaggccaggggccaagggcgatgcaggaccccccggagctcctgtcccatccacaccagctccccatgccagctgtggctccgaggaaaatcactcctacacgctatccctggagcctgagactgggtgccagcttcgccatgcctggacccagactgaatccccacacccttcctgggaaacagatacggtcccctgcccggtgatggacccaccatcaactgcatcctccaagctggattcctag

SEQ ID NO: 4 - DNA, 1778 bp, Homo sapiens
  CDS (273)...(1319)
  misc_feature (1)...(1778): n = A,T,C or G
gaatttgtaatacgactcactatagggagtcgacccacgcgtccggcgggtcggggaggaatattcttttggaaacgtaatattggccttggggctctccagccctttgggacttccaatgggatcttagaagcagccgaagcagcgtgagggcggcagcccagggccagccacgatttgaacgctctgccttgcagctcttctggaccgaggagcccaaagccctaccctcaccattcaccaggttacagttcttatccgcgtgaatacacatggctctgttacgaaaaattaatcaggtgctgctgttccttctgatcgtgaccctctgtgtgattctgtataagaaagttcataaggggactgtgcccaagaatgacgcagatgatgaatccgagactcctgaagaactggaagaagagattcctgtggtgatttgtgctgcagcagggaggatgggtgccactatggctgccatcaatagcatctacagcaacactgacgccaacatcttgttctatgtagtgggactccggaatactctgactcgaatacgaaaatggattgaacattccaaactgagagaaataaactttaaaatcgtggaattcaacccgatggtcctcaaagggaagatcagaccagactcatcgaggcctgaattgctccagcctctgaactttgttcgattttatctccctctacttatccaccaacacgagaaagtcatctatttggacgatgatgtaattgtacaaggtgatatccaagaactgtatgacaccaccttggccctgggccacgcggcggctttctcagatgactgcgatttgccctctgctcaggacataaacagactcgtgggacttcagaacacatatatgggctatctggactaccggaagaaggccatcaaggaccttggcatcagccccagcacctgctctttcaatcctggtgtgattgttgccaacatgacagaatggaagcaccagcgcatcaccaagcaattggagaaatggatgcaaaagaatgtggaggaaaacctctatagcagctccctgggaggaggggtggccacctccccaatgctgattgtgtttcatgggaaatattccacaattaaccccctgtggcacataaggcacctgggctggaatccagatgccagatattcggagcattttctgcaggaagctaaattactccactggaatggaagacataaaccttgggacttccctagtgttcacaacgacttatgggaaagctggtttgttcctgaccctgcagggatatttaaactcaatcaccatagctgatataactctacccttaaaatattccctgtatagaaatgtggaattgtccctttgtagccaactataacattgttctttatgaatattacctttgatacatatgatccacaatataaaaaccaaaaactactgtgtgcaaattataccttggaccatataggcattgattaacttctttaagtacatgtgataactatggaaatcaagattatgtgactgaaaaacataaaggaagagacccatctagataacagcaatcaacctgcttaattctgaatgacaattatatccacaaatttttaaaacttctacatgtatttttcacatgaagatctccttaacaggttgccaaccttttcttttataaaactattacatttaaaatatggacgtctgaaaaataaaatattcatcatttttaaaaaaaaaaaaaaaamaaaaaanaaaaaaaaaaaa

SEQ ID NO: 5 - protein, 349 aa, Homo sapiens
MALLRKINQVLLFLLIVTLCVILYKKVHKGTVPKNDADDESETPEELEEEIPVVICAAAGRMGATMAAINSIYSNTDANILFYVVGLRNTLTRIRKWIEHSKLREINFKIVEFNPMVLKGKIRPDSSRPELLQPLNFVRFYLPLLIHQHEKVIYLDDDVIVQGDIQELYDTTLALGHAAAFSDDCDLPSAQDINRLVGLQNTYMGYLDYRKKAIKDLGISPSTCSFNPGVIVANMTEWKHQRITKQLEKWMQKNVEENLYSSSLGGGVATSPMLIVFHGKYSTINPLWHIRHLGWNPDARYSEHFLQEAKLLHWNGRHKPWDFPSVHNDLWESWFVPDPAGIFKLNHHS

SEQ ID NO: 6 - DNA, 1050 bp, Homo sapiens
atggctctgttacgaaaaattaatcaggtgctgctgttccttctgatcgtgaccctctgtgtgattctgtataagaaagttcataaggggactgtgcccaagaatgacgcagatgatgaatccgagactcctgaagaactggaagaagagattcctgtggtgatttgtgctgcagcagggaggatgggtgccactatggctgccatcaatagcatctacagcaacactgacgccaacatcttgttctatgtagtgggactccggaatactctgactcgaatacgaaaatggattgaacattccaaactgagagaaataaactttaaaatcgtggaattcaacccgatggtcctcaaagggaagatcagaccagactcatcgaggcctgaattgctccagcctctgaactttgttcgattttatctccctctacttatccaccaacacgagaaagtcatctatttggacgatgatgtaattgtacaaggtgatatccaagaactgtatgacaccaccttggccctgggccacgcggcggctttctcagatgactgcgatttgccctctgctcaggacataaacagactcgtgggacttcagaacacatatatgggctatctggactaccggaagaaggccatcaaggaccttggcatcagccccagcacctgctctttcaatcctggtgtgattgttgccaacatgacagaatggaagcaccagcgcatcaccaagcaattggagaaatggatgcaaaagaatgtggaggaaaacctctatagcagctccctgggaggaggggtggccacctccccaatgctgattgtgtttcatgggaaatattccacaattaaccccctgtggcacataaggcacctgggctggaatccagatgccagatattcggagcattttctgcaggaagctaaattactccactggaatggaagacataaaccttgggacttccctagtgttcacaacgacttatgggaaagctggtttgttcctgaccctgcagggatatttaaactcaatcaccatagctga

SEQ ID NO: 7 - DNA, 2704 bp, Homo sapiens
  CDS (45)...(1844)
ggcgcgttcgagcagcggcgaccgacgcggcgaaggagcgcgccatggagcatgtgacagagggctcctgggagtcgctgcctgtgccgctgcacccgcaggtgctgggcgcgctgcgggagctgggcttcccgtacatgacgccggtgcagtccgcaaccatccctctgttcatgcgaaacaaagatgtcgctgcagaagcggtcacaggtagtggcaaaacactcgcttttgtcatccccatcctggaaattcttctgagaagagaagagaagttaaaaaagagtcaggttggagccataatcatcacccccactcgagagctggccattcaaatagacgaggtcctgtcgcatttcacgaagcacttccccgagttcagccagattctttggatcggaggcaggaatcctggagaagatgttgagaggtttaagcaacaaggtgggaacatcattgtggccactccaggccgcttggaggacatgttccggaggaaggccgaaggcttggatctggccagctgtgtgcgatccctggatgtcctggtgttggatgaggcagacagacttctggacatggggtttgaggcaagcatcaacaccattctggagtttttgccaaagcagaggagaacaggccttttctctgccactcagacgcaggaagtggagaacctggtgagagcgggcctccggaaccctgtccgggtctcagtgaaggagaagggcgtggcagccagcagtgcccagaagaccccctcccgcctggaaaactactacatggtatgcaaggcagatgagaaatttaatcagctggtccattttcttcgcaatcataagcaggagaaacacctggtcttcttcagcacctgtgcctgtgtggaatactatgggaaggctctggaagtgctggtgaagggcgtgaagattatgtgcattcacggaaagatgaaatataaacgcaataagatcttcatggagttccgcaaattgcaaagtgggattttagtgtgcactgatgtgatggcccggggaattgatattcctgaagtcaactgggttttgcagtatgaccctcccagcaatgcaagtgccttcgtgcatcgctgcggtcgcacagctcgcattggccacgggggcagcgctctggtgttcctcctgcccatggaagagtcatacatcaatttccttgcaattaaccaaaaatgccccctgcaggagatgaagccccagagaaacacagcggaccttctgccaaaactcaagtccatggccctggctgacagagctgtgtttgaaaagggcatgaaagcttttgtgtcatatgtccaagcttatgcaaagcatgaatgcaacctgattttcagattaaaggatcttgattttgccagccttgctcgaggttttgccctgctgaggatgcccaagatgccagaattgagaggaaagcagtttccagattttgtgcccgtggacgttaataccgacacgattccatttaaagataaaatcagagaaaagcagaggcagaaactcctggagcaacaaagaagagagaaaacagaaaatgaagggagaagaaaattcataaaaaataaagcttggtcaaagcagaaggccaaaaaagaaaagaagaaaaaaatgaatgagaaaaggaaaagggaagagggttctgatattgaagatgaggacatggaagaacttcttaatgacacaagactcttgaaaaaacttaagaaaggcaaaataactgaagaagaatttgagaagggcttgttgacaactggcaaaagaacaatcaagacagtggatttagggatctcagatttggaagatgactgctgattccagtgccacagatgaacccacaaggacatagctgttccctaacttggtggatggctccagtttgcttttaacgaaaatcacaacttcaggagacatctgaaaagaatgatgtctctgaaagctgtcctttcagatgagggagaaatgaaggatttcacacttcagaatattttactaaaaacattccagtcttggccgggtgcggtggctcctgcctataatcccagcactttgggaggctgaggcaggaggatcacttgagcccaggagttcaagaccagcctgggaacacagcgagaccctctcattaaaaacaacaaaacaaaacaattccagtcttggagtagtctaacagaagaaaatgtaaaattatttgagtgtaaataatagatgtcagtatttatcatgatgggtcacatatagacatatgthcatattatatatatatatatatatatatatatatatatatatatatatatatatataagctcttttttctgaggctattttatagttatttttaaacataaagayacasaagtcttcttsacttctgattttcacaaaccattcctcagtatcttcaggcatttgwcctcctgaatgtgcttggccmtgggcttcagttatcctttgatgtcctgcaggggtggctaatgtgctggggtttttctgtgttaatagtcmcagtattgttttattggtsaatagctgaamawcagagggattaagtcatattccgggaaagagaattatagtttttatgcctcctgttgaataaatggtgtcmtgattgcctgggtaaaaaaaaaaaaaawaaaggkcgkccgytagactatttagagaaaaaa

SEQ ID NO: 8 - protein, 600 aa, Homo sapiens
MEHVTEGSWESLPVPLHPQVLGALRELGFPYMTPVQSATIPLFMRNKDVAAEAVTGSGKTLAFVIPILEILLRREEKLKKSQVGAIIITPTRELAIQIDEVLSHFTKHFPEFSQILWIGGRNPGEDVERFKQQGGNIIVATPGRLEDMFRRKAEGLDLASCVRSLDVLVLDEADRLLDMGFEASINTILEFLPKQRRTGLFSATQTQEVENLVRAGLRNPVRVSVKEKGVAASSAQKTPSRLENYYMVCKADEKFNQLVHFLRNHKQEKHLVFFSTCACVEYYGKALEVLVKGVKIMCIHGKMKYKRNKIFMEFRKLQSGILVCTDVMARGIDIPEVNWVLQYDPPSNASAFVHRCGRTARIGHGGSALVFLLPMEESYINFLAINQKCPLQEMKPQRNTADLLPKLKSMALADRAVFEKGMKAFVSYVQAYAKHECNLIFRLKDLDFASLARGFALLRMPKMPELRGKQFPDFVPVDVNTDTIPFKDKIREKQRQKLLEQQRREKTENEGRRKFIKNKAWSKQKAKKEKKKKMNEKRKREEGSDIEDEDMEELLNDTRLLKKLKKGKITEEEFEKGLLTTGKRTIKTVDLGISDLEDDC

SEQ ID NO: 9 - DNA, 1803 bp, Homo sapiens
atggagcatgtgacagagggctcctgggagtcgctgcctgtgccgctgcacccgcaggtgctgggcgcgctgcgggagctgggcttcccgtacatgacgccggtgcagtccgcaaccatccctctgttcatgcgaaacaaagatgtcgctgcagaagcggtcacaggtagtggcaaaacactcgcttttgtcatccccatcctggaaattcttctgagaagagaagagaagttaaaaaagagtcaggttggagccataatcatcacccccactcgagagctggccattcaaatagacgaggtcctgtcgcatttcacgaagcacttccccgagttcagccagattctttggatcggaggcaggaatcctggagaagatgttgagaggtttaagcaacaaggtgggaacatcattgtggccactccaggccgcttggaggacatgttccggaggaaggccgaaggcttggatctggccagctgtgtgcgatccctggatgtcctggtgttggatgaggcagacagacttctggacatggggtttgaggcaagcatcaacaccattctggagtttttgccaaagcagaggagaacaggccttttctctgccactcagacgcaggaagtggagaacctggtgagagcgggcctccggaaccctgtccgggtctcagtgaaggagaagggcgtggcagccagcagtgcccagaagaccccctcccgcctggaaaactactacatggtatgcaaggcagatgagaaatttaatcagctggtccattttcttcgcaatcataagcaggagaaacacctggtcttcttcagcacctgtgcctgtgtggaatactatgggaaggctctggaagtgctggtgaagggcgtgaagattatgtgcattcacggaaagatgaaatataaacgcaataagatcttcatggagttccgcaaattgcaaagtgggattttagtgtgcactgatgtgatggcccggggaattgatattcctgaagtcaactgggttttgcagtatgaccctcccagcaatgcaagtgccttcgtgcatcgctgcggtcgcacagctcgcattggccacgggggcagcgctctggtgttcctcctgcccatggaagagtcatacatcaatttccttgcaattaaccaaaaatgccccctgcaggagatgaagccccagagaaacacagcggaccttctgccaaaactcaagtccatggccctggctgacagagctgtgtttgaaaagggcatgaaagcttttgtgtcatatgtccaagcttatgcaaagcatgaatgcaacctgattttcagattaaaggatcttgattttgccagccttgctcgaggttttgccctgctgaggatgcccaagatgccagaattgagaggaaagcagtttccagattttgtgcccgtggacgttaataccgacacgattccatttaaagataaaatcagagaaaagcagaggcagaaactcctggagcaacaaagaagagagaaaacagaaaatgaagggagaagaaaattcataaaaaataaagcttggtcaaagcagaaggccaaaaaagaaaagaagaaaaaaatgaatgagaaaaggaaaagggaagagggttctgatattgaagatgaggacatggaagaacttcttaatgacacaagactcttgaaaaaacttaagaaaggcaaaataactgaagaagaatttgagaagggcttgttgacaactggcaaaagaacaatcaagacagtggatttagggatctcagatttggaagatgactgctga

SEQ ID NO: 10 - DNA, 2737 bp, Homo sapiens
  CDS (69)...(2570)
cggtcggggcgttacggctgccgctcggcgccgcggtctcgtgccagtgagcgccgggcgccgcagccatgaccgtggagttcgaggagtgcgtcaaggactccccgcgcttcagggcgaccattgacgaggtggagacggacgtggtggagattgaggccaaactggacaagctggtgaagctgtgcagtggcatggtggaagccggtaaggcctacgtcagcaccagcaggcttttcgtgagcggcgtccgcgacctgtcccagcagtgccagggcgacaccgtcatctcggaatgtctgcagaggttcgctgacagcctacaggaggtggtgaactaccacatgatcctgtttgaccaggcccagaggtccgtgcggcagcagctccagagctttgtcaaagaggatgtgcggaagttcaaggagacaaagaagcagtttgacaaggtgcgggaggacctggagctgtccctggtgaggaacgcccaggccccgaggcaccggccccacgaggtggaggaagccaccggggccctcaccctcaccaggaagtgcttccgccacctggcactggactatgtgctccagatcaatgttctgcaggccaagaagaagtttgagatcctggactctatgctgtccttcatgcacgcccagtccagcttcttccagcagggctacagcctcctgcaccagctggacccctacatgaagaagctggcagccgagctggaccagctggtgatcgactctgcggtggaaaagcgtgagatggagcgaaagcacgccgccatccagcagcggacgctgctgcaggacttctcctacgatgagtccaaagtggagtttgacgtggacgcgcccagtggggtggtgatggagggctacctcttcaagagggccagcaacgctttcaagacatggaaccggcgctggttctccattcagaacagccagctggtctaccagaagaagctcaaggatgccctcaccgtggtggtggatgacctccgcctgtgctctgtgaagccgtgtgaggacatcgagcggaggttctgcttcgaggtgctgtcacccaccaagagctgcatgctgcaggctgactccgagaagctgcggcaagcctgggtccaggctgtgcaggccagcatcgcctccgcctaccgcgagagccctgacagttgctatagcgagaggctggaccgcacagcatccccgtccacgagcagcatcgactccgccaccgacactcgggagcgtggcgtgaagggcgagagtgtgctgcagcgtgtgcagagtgtggccggcaacagccagtgcggcgactgcggccagccggacccccgctgggccagcatcaacctgggcgtgctgctctgcattgagtgctccggcatccacaggagcctgggtgtccactgctccaaggtgcggtccctgacgctggactcgtgggagcctgagctgctaaagctgatgtgtgagcttggaaacagcgctgtgaatcagatctatgaggcccagtgtgagggtgcaggcagcaggaaacccacagccagcagctcccggcaggacaaggaggcctggatcaaggacaaatacgtggaaaagaagtttctgcggaaggcgcccatggcaccagccctggaggccccaagacgctggagggtgcagaagtgcctgcggccccacagctctccccgcgctcccactgcccgccgcaaggtccggcttgagcccgttctgccctgtgtggccgctctgtcctcagtgggcaccctggatcgtaagttccgccgagactccctcttctgtcccgacgagctggactcgctcttctcctacttcgacgcaggggccgcaggggctggccctcgcagtctgagtagcgacagtggccttgggggcagctcggatggcagctcggacgtcctggctttcggctcgggctctgtggtggacagcgtcactgaggaggagggtgcagagtcggaggagtccagcggtgaggcagacggggacactgaggccgaggcctggggcctggcggacgtgcgcgagctgcacccggggctcttggcgcaccgcgcagcgcgtgcccgcgaccttcctgcgctggcggcggcgctggcccacggggccgaggtcaactgggcggacgcggaggatgagggcaagacgccgctggtgcaggccgtgctagggggctccttgatcgtctgtgagttcctgctgcaaaacggagcggacgtgaaccaaagagacagccggggccgggcgcccctgcaccacgccacgctgctgggccgcaccggccaggtttgcctgttcctgaagcggggcgcggaccagcacgccctggaccaagagcagcgggacccgttggccatcgcagtgcaggcggccaacgctgacatcgtgacactgctccgtctggcgcgcatggcggaggaaatgcgcgaggccgaggctgcccctggtcccccgggcgccctggcgggcagccccacggagctccagttccgcaggtgtatccaggagttcatcagcctccacctggaagagagctagggccgggcgggccgggcagctgccaccccgcccggcccgacgccccgcatgccccgaagtccctggcgcccacccggccgcggccctgcgtgtgacccgcgggtcgatacctggcagccccagtgctggggcgccgcggccctgctcgcccaggaggagagcga

SEQ ID NO: 11 - protein, 834 aa, Homo sapiens
MTVEFEECVKDSPRFRATIDEVETDVVEIEAKLDKLVKLCSGMVEAGKAYVSTSRLFVSGVRDLSQQCQGDTVISECLQRFADSLQEVVNYHMILFDQAQRSVRQQLQSFVKEDVRKFKETKKQFDKVREDLELSLVRNAQAPRHRPHEVEEATGALTLTRKCFRHLALDYVLQINVLQAKKKFEILDSMLSFMHAQSSFFQQGYSLLHQLDPYMKKLAAELDQLVIDSAVEKREMERKHAAIQQRTLLQDFSYDESKVEFDVDAPSGVVMEGYLFKRASNAFKTWNRRWFSIQNSQLVYQKKLKDALTVVVDDLRLCSVKPCEDIERRFCFEVLSPTKSCMLQADSEKLRQAWVQAVQASIASAYRESPDSCYSERLDRTASPSTSSIDSATDTRERGVKGESVLQRVQSVAGNSQCGDCGQPDPRWASINLGVLLCIECSGIHRSLGVHCSKVRSLTLDSWEPELLKLMCELGNSAVNQIYEAQCEGAGSRKPTASSSRQDKEAWIKDKYVEKKFLRKAPMAPALEAPRRWRVQKCLRPHSSPRAPTARRKVRLEPVLPCVAALSSVGTLDRKFRRDSLFCPDELDSLFSYFDAGAAGAGPRSLSSDSGLGGSSDGSSDVLAFGSGSVVDSVTEEEGAESEESSGEADGDTEAEAWGLADVRELHPGLLAHRAARARDLPALAAALAHGAEVNWADAEDEGKTPLVQAVLGGSLIVCEFLLQNGADVNQRDSRGRAPLHHATLLGRTGQVCLFLKRGADQHALDQEQRDPLAIAVQAANADIVTLLRLARMAEEMREAEAAPGPPGALAGSPTELQFRRCIQEFISLHLEES

SEQ ID NO: 12 - DNA, 2505 bp, Homo sapiens
atgaccgtggagttcgaggagtgcgtcaaggactccccgcgcttcagggcgaccattgacgaggtggagacggacgtggtggagattgaggccaaactggacaagctggtgaagctgtgcagtggcatggtggaagccggtaaggcctacgtcagcaccagcaggcttttcgtgagcggcgtccgcgacctgtcccagcagtgccagggcgacaccgtcatctcggaatgtctgcagaggttcgctgacagcctacaggaggtggtgaactaccacatgatcctgtttgaccaggcccagaggtccgtgcggcagcagctccagagctttgtcaaagaggatgtgcggaagttcaaggagacaaagaagcagtttgacaaggtgcgggaggacctggagctgtccctggtgaggaacgcccaggccccgaggcaccggccccacgaggtggaggaagccaccggggccctcaccctcaccaggaagtgcttccgccacctggcactggactatgtgctccagatcaatgttctgcaggccaagaagaagtttgagatcctggactctatgctgtccttcatgcacgcccagtccagcttcttccagcagggctacagcctcctgcaccagctggacccctacatgaagaagctggcagccgagctggaccagctggtgatcgactctgcggtggaaaagcgtgagatggagcgaaagcacgccgccatccagcagcggacgctgctgcaggacttctcctacgatgagtccaaagtggagtttgacgtggacgcgcccagtggggtggtgatggagggctacctcttcaagagggccagcaacgctttcaagacatggaaccggcgctggttctccattcagaacagccagctggtctaccagaagaagctcaaggatgccctcaccgtggtggtggatgacctccgcctgtgctctgtgaagccgtgtgaggacatcgagcggaggttctgcttcgaggtgctgtcacccaccaagagctgcatgctgcaggctgactccgagaagctgcggcaagcctgggtccaggctgtgcaggccagcatcgcctccgcctaccgcgagagccctgacagttgctatagcgagaggctggaccgcacagcatccccgtccacgagcagcatcgactccgccaccgacactcgggagcgtggcgtgaagggcgagagtgtgctgcagcgtgtgcagagtgtggccggcaacagccagtgcggcgactgcggccagccggacccccgctgggccagcatcaacctgggcgtgctgctctgcattgagtgctccggcatccacaggagcctgggtgtccactgctccaaggtgcggtccctgacgctggactcgtgggagcctgagctgctaaagctgatgtgtgagcttggaaacagcgctgtgaatcagatctatgaggcccagtgtgagggtgcaggcagcaggaaacccacagccagcagctcccggcaggacaaggaggcctggatcaaggacaaatacgtggaaaagaagtttctgcggaaggcgcccatggcaccagccctggaggccccaagacgctggagggtgcagaagtgcctgcggccccacagctctccccgcgctcccactgcccgccgcaaggtccggcttgagcccgttctgccctgtgtggccgctctgtcctcagtgggcaccctggatcgtaagttccgccgagactccctcttctgtcccgacgagctggactcgctcttctcctacttcgacgcaggggccgcaggggctggccctcgcagtctgagtagcgacagtggccttgggggcagctcggatggcagctcggacgtcctggctttcggctcgggctctgtggtggacagcgtcactgaggaggagggtgcagagtcggaggagtccagcggtgaggcagacggggacactgaggccgaggcctggggcctggcggacgtgcgcgagctgcacccggggctcttggcgcaccgcgcagcgcgtgcccgcgaccttcctgcgctggcggcggcgctggcccacggggccgaggtcaactgggcggacgcggaggatgagggcaagacgccgctggtgcaggccgtgctagggggctccttgatcgtctgtgagttcctgctgcaaaacggagcggacgtgaaccaaagagacagccggggccgggcgcccctgcaccacgccacgctgctgggccgcaccggccaggtttgcctgttcctgaagcggggcgcggaccagcacgccctggaccaagagcagcgggacccgttggccatcgcagtgcaggcggccaacgctgacatcgtgacactgctccgtctggcgcgcatggcggaggaaatgcgcgaggccgaggctgcccctggtcccccgggcgccctggcgggcagccccacggagctccagttccgcaggtgtatccaggagttcatcagcctccacctggaagagagctag

SEQ ID NO: 13 - DNA, 1718 bp, Homo sapiens
  CDS (140)...(1192)
cacgcgtccgggcctgggggcgagctggggtcgtgcagtacagcctctttccggcaaatcacgcgagatttcgttcacccgggctccacacggagtattttatacagaaatcttgtgaaaccactgcccaaccagagcaatgattgttcaaagagtggtattgaattctcgacctggaaaaaatggtaatccagtggcagagaatttccgaatggaagaagtctatttaccagataatattaatgaaggacaagtacaagttagaactctttatctttctgtggatccttacatgcgttgtagaatgaatgaagacactggcactgattatataacaccttggcagctatctcaagtcgttgatggtggaggtattggaattatagaagaaagcaaacacacaaatttgactaaaggcgattttgtgacttctttctattggccctggcaaaccaaggttattctggatggaaatagccttgaaaaggtagacccacaacttgtggatggacacctttcatattttcttggagctataggtatgcctggtttgacttccttgattgggatacaggaaaaaggtcatataactgctggatctaataagacaatggttgtcagtggggccgcaggtgcctgtggatctgtggctgggcagattggccatttcttaggttgttccagagtggtgggaatttgtggaacacatgagaaatgcatcctcttgacctcagaactgggctttgatgctgcaattaattataaaaaagacaatgtggcagaacagctccgtgaatcatgcccagctggagtggatgtttattttgacaatgttggtggtaacatcagtgatacagtgataagtcagatgaatgagaacagccacatcatcctgtgtggtcaaatttctcagtacaacaaagatgtgccttatcctcccccgctatcccctgctatagaggcaatccagaaagaaagaaacatcacaagggaaagatttctggtattaaattataaagacaaatttgagcctggcattctacagctgagtcagtggtttaaagaaggaaagctaaagattaaagagacggtaataaatgggttggaaaacatgggagctgcattccagtccatgatgacaggaggtaacattggaaagcagatagtttgcatttcagaagaaatctctttgtaattgctgtaaatgtcatcaaggcaatcatagatttcttttccattttgcatattttcaaagatatgttaaaaaatccttagactatacatagctcttgatttaaatgtgatcataggtgttatttttagttgcatagggtatttgatacaatcattaatggatcatacacaataggtttttaaaaattaataacttttagtaattacttttattaatttaaaatagaacacttgagaggcactttgtaaagatttgttaaactggaaacgttttacatgatctgatacaaccattaatgaatcatacacaataggttttttaaaattaatattaataacttttattaatttaaaatagaatgcttaaaataaaatagaatgcttgagaggcactgagtaaagatttgttgaactggaaatgttttacatgatttttaaactgaaacttggtgtaaaaatagaattgagatggcctttttttcacattgtagactgaaaagagacttaatggtatgatgtgtacc

SEQ ID NO: 14 - protein, 351 aa, Homo sapiens
MIVQRVVLNSRPGKNGNPVAENFRMEEVYLPDNINEGQVQVRTLYLSVDPYMRCRMNEDTGTDYITPWQLSQVVDGGGIGIIEESKHTNLTKGDFVTSFYWPWQTKVILDGNSLEKVDPQLVDGHLSYFLGAIGMPGLTSLIGIQEKGHITAGSNKTMVVSGAAGACGSVAGQIGHFLGCSRVVGICGTHEKCILLTSELGFDAAINYKKDNVAEQLRESCPAGVDVYFDNVGGNISDTVISQMNENSHIILCGQISQYNKDVPYPPPLSPAIEAIQKERNITRERFLVLNYKDKFEPGILQLSQWFKEGKLKIKETVINGLENMGAAFQSMMTGGNIGKQIVCISEEISL

SEQ ID NO: 15 - DNA, 1056 bp, Homo sapiens
atgattgttcaaagagtggtattgaattctcgacctggaaaaaatggtaatccagtggcagagaatttccgaatggaagaagtctatttaccagataatattaatgaaggacaagtacaagttagaactctttatctttctgtggatccttacatgcgttgtagaatgaatgaagacactggcactgattatataacaccttggcagctatctcaagtcgttgatggtggaggtattggaattatagaagaaagcaaacacacaaatttgactaaaggcgattttgtgacttctttctattggccctggcaaaccaaggttattctggatggaaatagccttgaaaaggtagacccacaacttgtggatggacacctttcatattttcttggagctataggtatgcctggtttgacttccttgattgggatacaggaaaaaggtcatataactgctggatctaataagacaatggttgtcagtggggccgcaggtgcctgtggatctgtggctgggcagattggccatttcttaggttgttccagagtggtgggaatttgtggaacacatgagaaatgcatcctcttgacctcagaactgggctttgatgctgcaattaattataaaaaagacaatgtggcagaacagctccgtgaatcatgcccagctggagtggatgtttattttgacaatgttggtggtaacatcagtgatacagtgataagtcagatgaatgagaacagccacatcatcctgtgtggtcaaatttctcagtacaacaaagatgtgccttatcctcccccgctatcccctgctatagaggcaatccagaaagaaagaaacatcacaagggaaagatttctggtattaaattataaagacaaatttgagcctggcattctacagctgagtcagtggtttaaagaaggaaagctaaagattaaagagacggtaataaatgggttggaaaacatgggagctgcattccagtccatgatgacaggaggtaacattggaaagcagatagtttgcatttcagaagaaatctctttgtaa

SEQ ID NO: 16 - DNA, 1827 bp, Homo sapiens
  CDS (7)...(1461)
gcccttatgggccgctactctggcaagacgtgccggctgctcttcatgctggtgctcaccgtcgccttcttcgtggcggagctggtctccggctacctgggcaactccatcgcgctgctctccgactccttcaacatgctctccgacctgatctcgctgtgcgtgggcctgagcgccggctacatcgcccggcgccccacccggggcttcagcgccacctacggctacgcccgcgccgaggtggtgggcgcgctgagcaacgcggtcttcctcaccgcgctctgcttcaccatcttcgtggaggccgtgctgcgcctggcccggcccgagcgcatcgatgaccccgagctggtgctcatcgccggcgtcctggggctgttggtcaacgtggtggggctgctcatcttccaggactgcgccgcctggttcgcgtgctgcctccggggacgcagtcgccgcctgcagcagcggcagcagctggcggagggctgtgtccccggcgctttcggggggcctcagggcgcggaggacccgcggcgcgcggcggacccgacagccccaggctcggactcggccgtaaccctccgggggacctcggtggaaaggaagcgggagaagggggcgaccgtgttcgcaaacgtagcaggtgattccttcaacacccagaatgagccagaagacatgatgaaaaaagagaaaaagtctgaagctctgaatatcagaggtgtacttttgcatgtgatgggagatgccctggggtccgtggttgtggtcatcacggccatcatattctatgtgcttcccctgaagagtgaggacccgtgtaactggcagtgttacattgaccccagcctgactgtcctcatggtcatcatcattttgtcatctgccttcccgcttatcaaggagaccgctgccattctgctacagatggtcccaaaaggagtcaacatggaagagctgatgagtaaactctctgctgtgcctggaattagcagtgtacatgaagtgcacatctgggaacttgtaagtggaaagattattgccaccctgcacatcaagtatcctaaggacaggggatatcaagatgccagcacaaaaattcgagaaatcttccaccatgcgggaatccacaatgtgaccatccagtttgaaaatgtggacttgaaggaacccctggagcagaaggacttactgttgctctgcaactcaccctgcatctccaagggctgtgctaagcagctgtgttgtccccccggggcactgcctctggctcacgtcaatggctgtgctgagcaaaatggtgggccctctctagacacatacggaagtgatggcctcagtagaagagacgcaagagaagtggctattgaagtgtctttggatagctgtctgagtgaccacggacaaagtcttaacaaaactcaggaggaccaatgttatgtcaacagaacgcatttttaatctggtactcacataatcagaccatatagacgaggcactttggaaccacaagcttggctcacaaaaagagctttctgggttgtaggcccagactagacttgcagcatgcatgctctgtgttcactaggggttggctgtttgggattttagttaaacgtgtctgtgaatttttattgttaactaactcctttccattcccctgggtgtctcatgctgctctttgactgtttcagcttgaacatgcattttctaaagcaaactgcactagtgtatatatcagggacattaaagtctgggactggggctcaataaaaaaaaaaaaaaaaaaaaaaaaaaaaaaaaaaaaaaaaaaagggcggccgc

SEQ ID NO: 17 - protein, 485 aa, Homo sapiens
MGRYSGKTCRLLFMLVLTVAFFVAELVSGYLGNSIALLSDSFNMLSDLISLCVGLSAGYIARRPTRGFSATYGYARAEVVGALSNAVFLTALCFTIFVEAVLRLARPERIDDPELVLIAGVLGLLVNVVGLLIFQDCAAWFACCLRGRSRRLQQRQQLAEGCVPGAFGGPQGAEDPRRAADPTAPGSDSAVTLRGTSVERKREKGATVFANVAGDSFNTQNEPEDMMKKEKKSEALNIRGVLLHVMGDALGSVVVVITAIIFYVLPLKSEDPCNWQCYIDPSLTVLMVIIILSSAFPLIKETAAILLQMVPKGVNMEELMSKLSAVPGISSVHEVHIWELVSGKIIATLHIKYPKDRGYQDASTKIREIFHHAGIHNVTIQFENVDLKEPLEQKDLLLLCNSPCISKGCAKQLCCPPGALPLAHVNGCAEQNGGPSLDTYGSDGLSRRDAREVAIEVSLDSCLSDHGQSLNKTQEDQCYVNRTHF

SEQ ID NO: 18 - DNA, 1458 bp, Homo sapiens
atgggccgctactctggcaagacgtgccggctgctcttcatgctggtgctcaccgtcgccttcttcgtggcggagctggtctccggctacctgggcaactccatcgcgctgctctccgactccttcaacatgctctccgacctgatctcgctgtgcgtgggcctgagcgccggctacatcgcccggcgccccacccggggcttcagcgccacctacggctacgcccgcgccgaggtggtgggcgcgctgagcaacgcggtcttcctcaccgcgctctgcttcaccatcttcgtggaggccgtgctgcgcctggcccggcccgagcgcatcgatgaccccgagctggtgctcatcgccggcgtcctggggctgttggtcaacgtggtggggctgctcatcttccaggactgcgccgcctggttcgcgtgctgcctccggggacgcagtcgccgcctgcagcagcggcagcagctggcggagggctgtgtccccggcgctttcggggggcctcagggcgcggaggacccgcggcgcgcggcggacccgacagccccaggctcggactcggccgtaaccctccgggggacctcggtggaaaggaagcgggagaagggggcgaccgtgttcgcaaacgtagcaggtgattccttcaacacccagaatgagccagaagacatgatgaaaaaagagaaaaagtctgaagctctgaatatcagaggtgtacttttgcatgtgatgggagatgccctggggtccgtggttgtggtcatcacggccatcatattctatgtgcttcccctgaagagtgaggacccgtgtaactggcagtgttacattgaccccagcctgactgtcctcatggtcatcatcattttgtcatctgccttcccgcttatcaaggagaccgctgccattctgctacagatggtcccaaaaggagtcaacatggaagagctgatgagtaaactctctgctgtgcctggaattagcagtgtacatgaagtgcacatctgggaacttgtaagtggaaagattattgccaccctgcacatcaagtatcctaaggacaggggatatcaagatgccagcacaaaaattcgagaaatcttccaccatgcgggaatccacaatgtgaccatccagtttgaaaatgtggacttgaaggaacccctggagcagaaggacttactgttgctctgcaactcaccctgcatctccaagggctgtgctaagcagctgtgttgtccccccggggcactgcctctggctcacgtcaatggctgtgctgagcaaaatggtgggccctctctagacacatacggaagtgatggcctcagtagaagagacgcaagagaagtggctattgaagtgtctttggatagctgtctgagtgaccacggacaaagtcttaacaaaactcaggaggaccaatgttatgtcaacagaacgcatttttaa

SEQ ID NO: 19 - DNA, 2165 bp, Homo sapiens
  CDS (526)...(1485)
ccgggcaggtacgcggggaagctcttgagctcctctacctcttagaaagcacaattgaatcagatatcatatgaaagacatacacacttcatgtaatgctacctgcgagtctccctagaaaagcagtttttgtaggtgaaaacaatgaagccaggtaatattgcaaggaggctgtaattttagcagacctaccaacaacactgatgtaggaagctcattattttaatttctggagccttttaattttttctttagaaagtgtataaataattgcagtgctgctttgcttccaaaactgggcagtgagttcaacaacaacgacaacaacagccgcagctcatcctggccgtcatggagtttcttgaaagaacgtatcttgtgaatgataaagctgccaagatgtatgctttcacactagaaaggagctgcaaatgaacacttcatagcaatgtggaactccaacagaaaccggtgaataaagatcagtgtcccagagagagaccagaggagctggagtcaggaggcatgtaccactgccacagtggctccaagcccacagaaaagggggcgaatgagtacgcctatgccaagtggaaactctgttctgcttcagcaatatgcttcattttcatgattgcagaggtcgtgggtgggcacattgctgggagtcttgctgttgtcacagatgctgcccacctcttaattgacctgaccagtttcctgctcagtctcttctccctgtggttgtcatcgaagcctccctctaagcggctgacatttggatggcaccgagcagagatccttggtgccctgctctccatcctgtgcatctgggtggtgactggcgtgctagtgtacctggcatgtgagcgcctgctgtatcctgattaccagatccaggcgactgtgatgatcatcgtttccagctgcgcagtggcggccaacattgtactaactgtggttttgcaccagagatgccttggccacaatcacaaggaagtacaagccaatgccagcgtcagagctgcttttgtgcatgcccttggagatctatttcagagtatcagtgtgctaattagtgcacttattatctactttaagccagagtataaaatagccgacccaatctgcacattcatcttttccatcctggtcttggccagcaccatcactatcttaaaggacttctccatcttactcatggaaggtgtgccaaagagcctgaattacagtggtgtgaaagagcttattttagcagtcgacggggtgctgtctgtgcacagcctgcacatctggtctctaacaatgaatcaagtaattctctcagctcatgttgctacagcagccagccgggacagccaagtggttcggagagaaattgctaaagcccttagcaaaagctttacgatgcactcactcaccattcagatggaatctccagttgaccaggaccccgactgccttttctgtgaagacccctgtgactagctcagtcacaccgtcagtttcccaaatttgacaggccaccttcaaacatgctgctatgcagtttctgcatcatagaaaataaggaaccaaaggaagaaattcatgtcatggtgcaatgcatattttatctatttatttagttgcattcaccatgaaggaagaggcactgagatccatcaatcaattggattatatactgatcagtagctgtgttcaattgcaggaatgtgtatatagattattcctgagtggagccgaagtaacagctgtttgtaactatcggcaataccaaattcatctcccttccaataatgcatcttgagaacacataggtaaatttgaactcaggaaagtcttactagaaatcagtggaagggacaaatagtcacaaaattttaccaaaacattagaaacaaaaaataaggagagccaagtcaggaataaaagtgactctgtatgctaacgccacattagaacttggttctctcaccaagctgtaatgtgatttttttttctactctgaattggaaatatgtatgaatatacagagaagtgcttacaactaatttttatttacttgtcacattttggcaataaatccctcttatttctaaaaaaaaaaaaaaaaaaaaaaaaaaaaaaaaaaaaaaaaaaaaaaaaaaaaaaaaaaaaaaaaa

SEQ ID NO: 20 - protein, 320 aa, Homo sapiens
MYHCHSGSKPTEKGANEYAYAKWKLCSASAICFIFMIAEVVGGHIAGSLAVVTDAAHLLIDLTSFLLSLFSLWLSSKPPSKRLTFGWHRAEILGALLSILCIWVVTGVLVYLACERLLYPDYQIQATVMIIVSSCAVAANIVLTVVLHQRCLGHNHKEVQANASVRAAFVHALGDLFQSISVLISALIIYFKPEYKIADPICTFIFSILVLASTITILKDFSILLMEGVPKSLNYSGVKELILAVDGVLSVHSLHIWSLTMNQVILSAHVATAASRDSQVVRREIAKALSKSFTMHSLTIQMESPVDQDPDCLFCEDPCD

SEQ ID NO: 21 - DNA, 963 bp, Homo sapiens
atgtaccactgccacagtggctccaagcccacagaaaagggggcgaatgagtacgcctatgccaagtggaaactctgttctgcttcagcaatatgcttcattttcatgattgcagaggtcgtgggtgggcacattgctgggagtcttgctgttgtcacagatgctgcccacctcttaattgacctgaccagtttcctgctcagtctcttctccctgtggttgtcatcgaagcctccctctaagcggctgacatttggatggcaccgagcagagatccttggtgccctgctctccatcctgtgcatctgggtggtgactggcgtgctagtgtacctggcatgtgagcgcctgctgtatcctgattaccagatccaggcgactgtgatgatcatcgtttccagctgcgcagtggcggccaacattgtactaactgtggttttgcaccagagatgccttggccacaatcacaaggaagtacaagccaatgccagcgtcagagctgcttttgtgcatgcccttggagatctatttcagagtatcagtgtgctaattagtgcacttattatctactttaagccagagtataaaatagccgacccaatctgcacattcatcttttccatcctggtcttggccagcaccatcactatcttaaaggacttctccatcttactcatggaaggtgtgccaaagagcctgaattacagtggtgtgaaagagcttattttagcagtcgacggggtgctgtctgtgcacagcctgcacatctggtctctaacaatgaatcaagtaattctctcagctcatgttgctacagcagccagccgggacagccaagtggttcggagagaaattgctaaagcccttagcaaaagctttacgatgcactcactcaccattcagatggaatctccagttgaccaggaccccgactgccttttctgtgaagacccctgtgactag

SEQ ID NO: 22 - DNA, 2130 bp, Homo sapiens
  CDS (33)...(1415)
gcacgagggcgggagctgtgcagctccttatcatggggacaattcatctctttcgaaaaccacaaagatccttttttggcaagttgttacgggaatttagacttgtagcagctgaccgaaggtcctggaagatactgctctttggtgtaataaacttgatatgtactggcttcctgcttatgtggtgcagttctactaatagtatagctttaactgcctatacttacctgaccatttttgatctttttagtttaatgacatgtttaataagttactgggtaacattgaggaaacctagccctgtctattcatttgggtttgaaagattagaagtcctggctgtatttgcctccacagtcttggcacagttgggagctctctttatattaaaagaaagtgcagaacgctttttggaacagcccgagatacacacgggaagattattagttggtacttttgtggctctttgtttcaacctgttcacgatgctttctattcggaataaaccttttgcttatgtctcagaagctgctagtacgagctggcttcaagagcatgttgcagatcttagtcgaagcttgtgtggaattattccgggacttagcagtatcttccttccccgaatgaatccatttgttttgattgatcttgctggagcatttgctctttgtattacatatatgctcattgaaattaataattattttgccgtagacactgcctctgctatagctattgccttgatgacatttggcactatgtatcccatgagtgtgtacagtgggaaagtcttactccagacaacaccaccccatgttattggtcagttggacaaactcatcagagaggtatctaccttagatggagttttagaagtccgaaatgaacatttttggaccctaggttttggctcattggctggatcagtgcatgtaagaattcgacgagatgccaatgaacaaatggttcttgctcatgtgaccaacaggctgtacactctagtgtctactctaactgttcaaattttcaaggatgactggattaggcctgccttattgtctgggcctgttgcagccaatgtcctaaacttttcagatcatcacgtaatcccaatgcctcttttaaagggtactgatgatttgaacccagttacatcaactccagctaaacctagtagtccacctccagaattttcatttaacactcctgggaaaaatgtgaacccagttattcttctaaacacacaaacaaggccttatggttttggtctcaatcatggacacacaccttacagcagcatgcttaatcaaggacttggagttccaggaattggagcaactcaaggattgaggactggttttacaaatataccaagtagatatggaactaataatagaattggacaaccaagaccatgatagactctaacttatttttataaggaatattgactccttggcttccaatttatttagtaatccaactttgcattgactgtttaatcatttactctaaatgttagataatagtagtcttgttcacatttcatgaaacctatgaaactatatttttgtaaaatgtatttgtgacagtgaaatcctcgtaaatgttaaaggctttaaataggcttcctttagaaaatgtgtttctttaaatttggattttggtatctttggttttgtagttgactgcagtgtgatgtgaccttacctttataagagccacttgatggagtagatctgtcacattactaagatacgatatttctttttttttccgagacggagtcttgctctgccactgtgcccggccaatacattattattaacttaaggctgtactttattaaggcttccttagtttttgttttgttttgttttttgagatggagtctcactctgtcgcccaggctggaatgcagtggcatgatctcagctcactgcaacctctgcctcctgaggcaggagagttgcttgaacctggggggcggaggttgcagtgagccactgcactccagcctgatgacagagcaagactcagtctcaaaaataaataaaaataataaaacctccataagtaatcctgaaaaaaaaaaaaaaaaaaaaatccgagggggggcgccggtaccca

SEQ ID NO: 23 - protein, 461 aa, Homo sapiens
MGTIHLFRKPQRSFFGKLLREFRLVAADRRSWKILLFGVINLICTGFLLMWCSSTNSIALTAYTYLTIFDLFSLMTCLISYWVTLRKPSPVYSFGFERLEVLAVFASTVLAQLGALFILKESAERFLEQPEIHTGRLLVGTFVALCFNLFTMLSIRNKPFAYVSEAASTSWLQEHVADLSRSLCGIIPGLSSIFLPRMNPFVLIDLAGAFALCITYMLIEINNYFAVDTASAIAIALMTFGTMYPMSVYSGKVLLQTTPPHVIGQLDKLIREVSTLDGVLEVRNEHFWTLGFGSLAGSVHVRIRRDANEQMVLAHVTNRLYTLVSTLTVQIFKDDWIRPALLSGPVAANVLNFSDHHVIPMPLLKGTDDLNPVTSTPAKPSSPPPEFSFNTPGKNVNPVILLNTQTRPYGFGLNHGHTPYSSMLNQGLGVPGIGATQGLRTGFTNIPSRYGTNNRIGQPRP

SEQ ID NO: 24 - DNA, 1386 bp, Homo sapiens
atggggacaattcatctctttcgaaaaccacaaagatccttttttggcaagttgttacgggaatttagacttgtagcagctgaccgaaggtcctggaagatactgctctttggtgtaataaacttgatatgtactggcttcctgcttatgtggtgcagttctactaatagtatagctttaactgcctatacttacctgaccatttttgatctttttagtttaatgacatgtttaataagttactgggtaacattgaggaaacctagccctgtctattcatttgggtttgaaagattagaagtcctggctgtatttgcctccacagtcttggcacagttgggagctctctttatattaaaagaaagtgcagaacgctttttggaacagcccgagatacacacgggaagattattagttggtacttttgtggctctttgtttcaacctgttcacgatgctttctattcggaataaaccttttgcttatgtctcagaagctgctagtacgagctggcttcaagagcatgttgcagatcttagtcgaagcttgtgtggaattattccgggacttagcagtatcttccttccccgaatgaatccatttgttttgattgatcttgctggagcatttgctctttgtattacatatatgctcattgaaattaataattattttgccgtagacactgcctctgctatagctattgccttgatgacatttggcactatgtatcccatgagtgtgtacagtgggaaagtcttactccagacaacaccaccccatgttattggtcagttggacaaactcatcagagaggtatctaccttagatggagttttagaagtccgaaatgaacatttttggaccctaggttttggctcattggctggatcagtgcatgtaagaattcgacgagatgccaatgaacaaatggttcttgctcatgtgaccaacaggctgtacactctagtgtctactctaactgttcaaattttcaaggatgactggattaggcctgccttattgtctgggcctgttgcagccaatgtcctaaacttttcagatcatcacgtaatcccaatgcctcttttaaagggtactgatgatttgaacccagttacatcaactccagctaaacctagtagtccacctccagaattttcatttaacactcctgggaaaaatgtgaacccagttattcttctaaacacacaaacaaggccttatggttttggtctcaatcatggacacacaccttacagcagcatgcttaatcaaggacttggagttccaggaattggagcaactcaaggattgaggactggttttacaaatataccaagtagatatggaactaataatagaattggacaaccaagaccatga

SEQ ID NO: 25 - DNA, 3304 bp, Homo sapiens
  CDS (273)...(2567)
  misc_feature (1)...(3304): n = A,T,C or G
ttgctgggcctgatgacgtggcttggcaacgtccctaccgccgctgcttcccgggaacctggcgccgccggaactgatcgcggcctagtcccgacgcgtgtgtgctagtgagccggagccggcgacggcggcagtggcggcccggcctgcaggagcccgacggggtctctgccatgggggagtgacgcgcctgcacccgctgttccgcggcagcggcgagacatgaggagaccccgcgacaggggcagcggcggcggctcgtgagccccgggatggaggagaaatacggcggggacgtgctggccggccccggcggcggcggcggccttgggccggtggacgtacccagcgctcgattaacaaaatatattgtgttactatgtttcactaaatttttgaaggctgtgggacttttcgaatcatatgatctcctaaaagctgttcacattgttcagttcatttttatattaaaacttgggactgcattttttatggttttgtttcaaaagccattttcttctgggaaaactattaccaaacaccagtggatcaaaatatttaaacatgcagttgctgggtgtattatttcactcttgtggttttttggcctcactctttgtggaccactaaggactttgctgctatttgagcacagtgatattgttgtcatttcactactcagtgttttgttcaccagttctggaggaggaccagcaaagacaaggggagctgcttttttcattattgctgtgatctgtttattgctttttgacaatgatgatctcatggctaaaatggctgaacaccctgaaggacatcatgacagtgctctaactcatatgctttacacagccattgccttcttaggtgtggcagatcacaagggtggagtattattgctagtactggctttgtgttgtaaagttggttttcatacagcttccagaaagctctctgtcgacgttggtggagctaaacgtcttcaagctttatctcatcttgtttctgtgcttctcttgtgcccatgggtcattgttctttctgtgacaactgagagtaaagtggagtcttggttttctctcattatgccttttgcaacggttatcttttttgtcatgatcctggatttctacgtggattccatttgttcagtcaaaatggaagtttccaaatgtgctcgttatggatcctttcccatttttattagtgctctcctttttggaaatttttggacacatccaataacagaccagcttcgggctatgaacaaagcagcacaccaggagagcactgaacacgtcctgtctggaggagtggtagtgagtgctatattcttcattttgtctgccaatatcttatcatctccctctaagagaggacaaaaaggtacccttattggatattctcctgaaggaacacctctttataacttcatgggtgatgcttttcagcatagctctcaatcgatccctaggtttattaaggaatcactaaaacaaattcttgaggagagtgactctaggcagatcttttacttcttgtgcttgaatctgctttttacctttgtggaattattctatggcgtgctgaccaatagtctgggcctgatctcggatggattccacatgctttttgactgctctgctttagtcatgggactttttgctgccctgatgagtaggtggaaagccactcggattttctcctatgggtacggccgaatagaaattctgtctggatttattaatggactttttctaatagtaatagcgttttttgtgtttatggagtcagtggctarattgattgatcctccagaattagacactcacatgttaacaccagtytcagttggagggctgatagtaaaccttattggtatctgtgcctttagccatgcccatagccatgcccatggagcttctcaaggaagctgtcactcatctgatcacagccattcacaycatatgcatggacacagtgaccatgggcatggtcacagccacggatctgcgggtggaggcatgaatgctaacatgaggggtgtatttctacatgttttggcagatacacttggcagcattggtgtgatcgtatccacagttcttatagagcagtttggatggttcatcgctgacccactctgttctctttttattgctatattaatatttctcagtgttgttccactgattaaagatgcctgccaggttctactcctgagattgccaccagaatatgaaaaagaactacatattgctttagaaaaggtacaggaaattgaaggattaatatcataccgagaccctcatttttggcgtcattytgctagtattgtggcaggaacaattcatatacaggtgacatytgatgtgctagaacaaagaatagtacrgcaggttacaggaatacttaaagatgctggagtaaacaatttaacaattcaagtggaaaaggaggcatactttcaacatatgtctggcctaagtactggatttcatgatgttctggctatgacaaaacaaatggaatccatgaaatactgcaaagatggtacttacatcatgtgagataactcaagrattacccctggrgrataaacaatgaagrttaaatgactcagtatttgtaatattgccagaaggataaaaattacacattaactgtacagaaacagagttccctactactggatcaaggaatctttcttgaaggaaatttaaatacagaatgaaacattaatggtaaaagtggagtaattatttaaattatgtgtataaaaggaatcaaattttgagtaaacatgatgtattacatcatcttcaaaaatagatatgatggattctagtgaagaccaaaattacttctgtttactttctatcaggaagcatctccattgtaaatatgtatttacatgtttattacaaagacccaaatgaaaaatttttagtccattttttgcatagcctaaagataaaataggaataaaagttctatatttatgggattttctgtatataaaactggtttctaattataacttaagtccattaagtaaaatctgtattgccactttaaatgtaaactaaattatttgggagaaacttcaaccactgatatgagataagcaatgagaatagggaagtgtataacatcacagtttttgatgtattacaaaaatcaaccactttataaaataaattttttttacttttggtaaaaaaaaaaaaaaaaaaaaaaaaaaaaaaaaaaaaaaaaagcggccgctgaattctagntagaattcagcggccgctgaatncta

SEQ ID NO: 26 - protein, 765 aa, Homo sapiens
  VARIANT (1)...(765): Xaa = Any Amino Acid
MEEKYGGDVLAGPGGGGGLGPVDVPSARLTKYIVLLCFTKFLKAVGLFESYDLLKAVHIVQFIFILKLGTAFFMVLFQKPFSSGKTITKHQWIKIFKHAVAGCIISLLWFFGLTLCGPLRTLLLFEHSDIVVISLLSVLFTSSGGGPAKTRGAAFFIIAVICLLLFDNDDLMAKMAEHPEGHHDSALTHMLYTAIAFLGVADHKGGVLLLVLALCCKVGFHTASRKLSVDVGGAKRLQALSHLVSVLLLCPWVIVLSVTTESKVESWFSLIMPFATVIFFVMILDFYVDSICSVKMEVSKCARYGSFPIFISALLFGNFWTHPITDQLRAMNKAAHQESTEHVLSGGVVVSAIFFILSANILSSPSKRGQKGTLIGYSPEGTPLYNFMGDAFQHSSQSIPRFIKESLKQILEESDSRQIFYFLCLNLLFTFVELFYGVLTNSLGLISDGFHMLFDCSALVMGLFAALMSRWKATRIFSYGYGRIEILSGFINGLFLIVIAFFVFMESVAXLIDPPELDTHMLTPXSVGGLIVNLIGICAFSHAHSHAHGASQGSCHSSDHSHSHHMHGHSDHGHGHSHGSAGGGMNANMRGVFLHVLADTLGSIGVIVSTVLIEQFGWFIADPLCSLFIAILIFLSVVPLIKDACQVLLLRLPPEYEKELHIALEKVQEIEGLISYRDPHFWRHXASIVAGTIHIQVTXDVLEQRIVXQVTGILKDAGVNNLTIQVEKEAYFQHMSGLSTGFHDVLAMTKQMESMKYCKDGTYIM

SEQ ID NO: 27 - DNA, 2298 bp, Homo sapiens
atggaggagaaatacggcggggacgtgctggccggccccggcggcggcggcggccttgggccggtggacgtacccagcgctcgattaacaaaatatattgtgttactatgtttcactaaatttttgaaggctgtgggacttttcgaatcatatgatctcctaaaagctgttcacattgttcagttcatttttatattaaaacttgggactgcattttttatggttttgtttcaaaagccattttcttctgggaaaactattaccaaacaccagtggatcaaaatatttaaacatgcagttgctgggtgtattatttcactcttgtggttttttggcctcactctttgtggaccactaaggactttgctgctatttgagcacagtgatattgttgtcatttcactactcagtgttttgttcaccagttctggaggaggaccagcaaagacaaggggagctgcttttttcattattgctgtgatctgtttattgctttttgacaatgatgatctcatggctaaaatggctgaacaccctgaaggacatcatgacagtgctctaactcatatgctttacacagccattgccttcttaggtgtggcagatcacaagggtggagtattattgctagtactggctttgtgttgtaaagttggttttcatacagcttccagaaagctctctgtcgacgttggtggagctaaacgtcttcaagctttatctcatcttgtttctgtgcttctcttgtgcccatgggtcattgttctttctgtgacaactgagagtaaagtggagtcttggttttctctcattatgccttttgcaacggttatcttttttgtcatgatcctggatttctacgtggattccatttgttcagtcaaaatggaagtttccaaatgtgctcgttatggatcctttcccatttttattagtgctctcctttttggaaatttttggacacatccaataacagaccagcttcgggctatgaacaaagcagcacaccaggagagcactgaacacgtcctgtctggaggagtggtagtgagtgctatattcttcattttgtctgccaatatcttatcatctccctctaagagaggacaaaaaggtacccttattggatattctcctgaaggaacacctctttataacttcatgggtgatgcttttcagcatagctctcaatcgatccctaggtttattaaggaatcactaaaacaaattcttgaggagagtgactctaggcagatcttttacttcttgtgcttgaatctgctttttacctttgtggaattattctatggcgtgctgaccaatagtctgggcctgatctcggatggattccacatgctttttgactgctctgctttagtcatgggactttttgctgccctgatgagtaggtggaaagccactcggattttctcctatgggtacggccgaatagaaattctgtctggatttattaatggactttttctaatagtaatagcgttttttgtgtttatggagtcagtggctarattgattgatcctccagaattagacactcacatgttaacaccagtytcagttggagggctgatagtaaaccttattggtatctgtgcctttagccatgcccatagccatgcccatggagcttctcaaggaagctgtcactcatctgatcacagccattcacaycatatgcatggacacagtgaccatgggcatggtcacagccacggatctgcgggtggaggcatgaatgctaacatgaggggtgtatttctacatgttttggcagatacacttggcagcattggtgtgatcgtatccacagttcttatagagcagtttggatggttcatcgctgacccactctgttctctttttattgctatattaatatttctcagtgttgttccactgattaaagatgcctgccaggttctactcctgagattgccaccagaatatgaaaaagaactacatattgctttagaaaaggtacaggaaattgaaggattaatatcataccgagaccctcatttttggcgtcattytgctagtattgtggcaggaacaattcatatacaggtgacatytgatgtgctagaacaaagaatagtacrgcaggttacaggaatacttaaagatgctggagtaaacaatttaacaattcaagtggaaaaggaggcatactttcaacatatgtctggcctaagtactggatttcatgatgttctggctatgacaaaacaaatggaatccatgaaatactgcaaagatggtacttacatcatgtga

SEQ ID NO: 28 - DNA, 2637 bp, Homo sapiens
  CDS (165)...(1292)
gtcgacccacgcgtccggtctgtgtctgtctgtgtctcgcagcggcgcgcggccccggacaagcgctggggattcccgtttgaggcgtcactactgtcactgccatcaccccacggagccacttctagaggggagtagacccggcccttcgccgggcagagaagatgttgcccctgtccatcaaagacgatgaatacaaaccacccaagttcaatttgttcggcaagatctcgggctggtttaggtctatactgtccgacaagacttcccggaacctgtttttcttcctgtgcctgaacctctctttcgcttttgtggaactactctacggcatctggagcaactgcttaggcttgatttccgactcttttcacatgtttttcgatagcactgccattttggctggactggcagcttctgttatttcaaaatggagagataatgatgctttctcctatgggtatgttagagcggaagttctggctggctttgtcaatggcctatttttgatcttcactgctttttttattttctcagaaggagttgagagagcattagcccctccagatgtccaccatgagagactgcttcttgtttccattcttgggtttgtggtaaacctaataggaatatttgttttcaaacatggaggtcatggacattctcatggctctggccacggacacagtcattccctctttaatggtgctctagatcaggcacatggccatgtcgatcattgccatagccatgaagtgaaacatggtgctgcacatagccatgatcatgctcatggacatggacactttcattctcatgatggcccgtccttaaaagaaacaacaggacccagcagacagattttacaaggtgtatttttacatatcctagcagatacacttggaagtattggtgtaattgcttctgccatcatgatgcaaaattttggtctgatgatagcagatcctatctgttcaattcttatagccattcttatagttgtaagtgttattcctcttttaagagaatctgttggaatattaatgcagagaactcctcccctattagaaaatagtctgcctcagtgctatcagagggtacagcagttgcaaggagtttacagtttacaggaacagcacttctggactttatgttctgacgtttatgttgggaccttgaaattaatagtagcacctgatgctgatgctaggtggattttaagccaaacacataatatttttactcaggctggagtgagacagctctacgtacagattgactttgcagccatgtagtgaatggaaagaaattatgcaccttttatggaccaaatttttctgccagtaagaatttcagttgtgggcctccagtcttctggaatgtcttcactgcagctgctggaaatcactgctttcattcccacaaaaccagtattacttttttttaaaaaaagaaagaaattggaaatctgtgctatacgtaatgtcataattgttgacgttcttcagtataatcatgtttgttaaattgtttgtaccttgcaaacttatgaaccaaaccatattgggttttcaaagtgcctttgtatccaaaaatgtatttgccagcatagaaatgtaccatcaagagtcatgtatgttttatttttgtttttattttttattttttgagatggagtctcgctctgtagccaggctggagtgcagtggcataatctcagctcactgcaacctctgcttcccgggttcaagctattctcctgcctcagccttctgagtagctgcgactatgggcgtgtgccaccatgcccagctaatttttgcatttttagtagagatggggtttcaccatgttggccaggatggtcttgatctcttgacctcgtgatccgtccacctcagcctcccaaagtgctgggattacaggtgtgagccaccacgcccggctatgttttttttttttttttttttttaacaatggatattctgtaaaatatcctgtacaaaacaagactattgccagtaatgtatacaaattgtctttttgtgtacttccagcataggtttggatatatgaacatttttcttttattgttttatcttcacagaaaataaaaggtgttaatttgctttttaaaacaaatttaattatcacattttaaattactttgtggactgtgttttcaaaactttgcaaattcatctgttacacagaaaattttgacttaaaacatctgctgaattccaaatttctgtaatagctactgtatctgtgataaactttcctatatctttccttgccatttctatggattttataatgaaagaaaaaggcattggagactgaaggcagaaatggttgtgacagtgctgtttggctttttcattcttcaaatgccaagtcatccactttgttttcctgtttaggctttgcacaaatacaattgctttcaggaatcctaaagcagcattttattgagtttgaattattaaaggtacagaggaaatgtggtgatgtagaacttttcctaacacaggtatctaggaagtaagtgctgagttgattttctaggttcttacgtatttgaaaaataaaattgcaattcgagataaaaaaaaaaaaaaaaagggcggccgc

SEQ ID NO: 29 - protein, 376 aa, Homo sapiens
MLPLSIKDDEYKPPKFNLFGKISGWFRSILSDKTSRNLFFFLCLNLSFAFVELLYGIWSNCLGLISDSFHMFFDSTAILAGLAASVISKWRDNDAFSYGYVRAEVLAGFVNGLFLIFTAFFIFSEGVERALAPPDVHHERLLLVSILGFVVNLIGIFVFKHGGHGHSHGSGHGHSHSLFNGALDQAHGHVDHCHSHEVKHGAAHSHDHAHGHGHFHSHDGPSLKETTGPSRQILQGVFLHILADTLGSIGVIASAIMMQNFGLMIADPICSILIAILIVVSVIPLLRESVGILMQRTPPLLENSLPQCYQRVQQLQGVYSLQEQHFWTLCSDVYVGTLKLIVAPDADARWILSQTHNIFTQAGVRQLYVQIDFAAM

SEQ ID NO: 30 - DNA, 1131 bp, Homo sapiens
atgttgcccctgtccatcaaagacgatgaatacaaaccacccaagttcaatttgttcggcaagatctcgggctggtttaggtctatactgtccgacaagacttcccggaacctgtttttcttcctgtgcctgaacctctctttcgcttttgtggaactactctacggcatctggagcaactgcttaggcttgatttccgactcttttcacatgtttttcgatagcactgccattttggctggactggcagcttctgttatttcaaaatggagagataatgatgctttctcctatgggtatgttagagcggaagttctggctggctttgtcaatggcctatttttgatcttcactgctttttttattttctcagaaggagttgagagagcattagcccctccagatgtccaccatgagagactgcttcttgtttccattcttgggtttgtggtaaacctaataggaatatttgttttcaaacatggaggtcatggacattctcatggctctggccacggacacagtcattccctctttaatggtgctctagatcaggcacatggccatgtcgatcattgccatagccatgaagtgaaacatggtgctgcacatagccatgatcatgctcatggacatggacactttcattctcatgatggcccgtccttaaaagaaacaacaggacccagcagacagattttacaaggtgtatttttacatatcctagcagatacacttggaagtattggtgtaattgcttctgccatcatgatgcaaaattttggtctgatgatagcagatcctatctgttcaattcttatagccattcttatagttgtaagtgttattcctcttttaagagaatctgttggaatattaatgcagagaactcctcccctattagaaaatagtctgcctcagtgctatcagagggtacagcagttgcaaggagtttacagtttacaggaacagcacttctggactttatgttctgacgtttatgttgggaccttgaaattaatagtagcacctgatgctgatgctaggtggattttaagccaaacacataatatttttactcaggctggagtgagacagctctacgtacagattgactttgcagccatgtag

SEQ ID NO: 31 - protein, 227 aa, Artificial Sequence
  consensus sequence
LLLDPLELAKQLTLLEHELFKKITPFECLGSKMKHEGPQVWGKKSLKGKEERSPNIDKTIKNFNQLTNFVSETILKQEFPTNPEFSKKVDPKKRAEVIQKFIQVADHCRELNNFNSLLAIISALYSSPIYRLKKTWEKVPPQSLKLIFEELNKLMDSDRNFSNYRELLKSIGPLKPPNLDDDFVRAPCVPFFGVYLSDLTFLEEGNPDYLENRKINTNLVNFSKRRK

SEQ ID NO: 32 - protein, 261 aa, Artificial Sequence
  consensus sequence
LLLLDPEELAEQLTLLDFELFRKIEPSELLGSVWGKRSKKSPSPLAPQNLEAFIERFNEVSNWVATEILKQTTLKPKKRAEVLSKFIKVAKHCRELNNFNSLMAIVSALSSSPISRLKKTWEKLSSKYKKLFEELEELLDPSERNFKNYREALKSCNKSPNVQPPCVPFLGVYLKDLTFIDEGNPDFLENGTKGLVNFEKRRKIAKILREIRQLQSACQPYNLKPNRNDIQELLRASRPLEVLPEEEDELYELSLRIEPRV

SEQ ID NO: 33 - protein, 172 aa, Artificial Sequence
  consensus sequence
CKGGLIKGGTLEKLIEHLTEARDKGKVDLKADPTFVETFLLTYRSFITTQELLQKLLDRYNAIPPEIVSRSLELKATEASEGLSFEDYSKNAIPVGFAETDIIVKEKVNPRRIQNRVLNILRLWVENYWEDFEEDPKLNLFLEELFLELVEDKKDKGPGLEKSLANILERLS

SEQ ID NO: 34 - protein, 29 aa, Artificial Sequence
  consensus sequence
ELKEAFKEFDKDGDGKISFEEFKAALKKL

SEQ ID NO: 35 - protein, 29 aa, Artificial Sequence
  consensus sequence
ELKEAFREFDKDGDGKIDFEEFKALLKSL

SEQ ID NO: 36 - protein, 51 aa, Artificial Sequence
  consensus sequence
HRFKRTTFYKSPTFCDHCGELLWGLAKQGLKCSNCGLNVHKRCHEKVPTNC

SEQ ID NO: 37 - protein, 61 aa, Artificial Sequence
  consensus sequence
HKFVFRTFKPTFCDVCRKSIWGSFKQAAKSQGLRCSECKVKCHKKCAEKVPAQSHKSGLSC

SEQ ID NO: 38 - protein, 334 aa, Artificial Sequence
  consensus sequence
KGVGVAINSLLKNLSAEENVKFHILTDGFKEKSRLLVSQGDNKKLLETLAPPEFQHFTAIRFYDINTSDELLESLPLTANMKRRISKATYAREDIQVLLLADLLPDYSKKRVLYLDADVVVKGADLSELLDLDLGENKILGAVEDLVSNESKFFFVDFWQERAKSRLIEYEQFLYETDGLKVIGSYFNAGVLLINLKKWREENLFKKALELLNEKKEEDVYFYPDQDVLNILFKGKFKSDEIDSIARVLFLPPRYNTQYSNVAYMYYFLKWRHKEKLAKKLLNPTKGNPSIVHYCGAGTKPWHKTKLKYPKKRVEYFDEAFLNSPWKDLYLKPK

SEQ ID NO: 39 - protein, 149 aa, Artificial Sequence
  consensus sequence
KALKKLGFEKPTPIQAEAIPAILEDVLVQAPTGSGKTLAFLLPILERLLLVLAPTRELANQIYEELKKLILVATPGRLLDHLENGSLLEKRLKLKNLKLLVLDEADRMLDMGKAHGFGPDLEEQTLLFSATLPEVERLAKLFLLRIKQK

SEQ ID NO: 40 - protein, 219 aa, Artificial Sequence
  consensus sequence
GFKLRPYQKEAIPWLLEGRDVILAAPTGSGKTLAFLLPLLELLKKGKPLASKKGKGPRVLVLVPTRELAEQWAEELKKLGPSLGVYFVEVLKVVGLYGGSKTKEKEQLRKLKSGKTDILVTTPGRLLDLLNGKGLSLNLDLVILDEAHRLLDMGSGGDHGFGDQLELLKRLPKNRQRLLLSATPPEIENLLKLFLNLPELFPNPVFISVGFTLEPIEQF

SEQ ID NO: 41 - protein, 87 aa, Artificial Sequence
  consensus sequence
DELAKFLKELFPKLPGIKVARLHGGLSQEEREEILEKFRNGKSKVLVATDVAARGIDIPDVNLVINYDLPWNPESYIQRIGRAGRAG

SEQ ID NO: 42 - protein, 86 aa, Artificial Sequence
  consensus sequence
DELAELLKELLKDPGIKVARLHGGLSQEEREEILEKFRKGKIKVLVATDVAERGLDIPGVDLVINYDLPWSPASYIQRIGRAGRAG

SEQ ID NO: 43 - protein, 85 aa, Artificial Sequence
  consensus sequence
VIKEGWLLKKSKSWKKRYFVLFNNVLLYYKDSKKKPKGSIPLSGCQVEKPDKNCFEIRTDRTLLLQAESEEERKEWVKAIQSAIR

SEQ ID NO: 44 - protein, 138 aa, Artificial Sequence
  consensus sequence
QRVLKLLRSLDPGNKKCFDCGAVPNPTWASVNLGVFLCIECSGIHRNLFGVHISKVNVRSLTLDTWTPEELRKLESCKGGNENANSFWESNLDDFSLKPKDSDAAAVKCQDDRQKYESFIAAKYEEKLFRVLKESKEE

SEQ ID NO: 45 - protein, 33 aa, Artificial Sequence
  consensus sequence
DGRTPLHLAARNGHLEVVKLLLEAGADVNARDK

SEQ ID NO: 46 - protein, 457 aa, Artificial Sequence
  consensus sequence
PLEVEEVPVPEPGPGEVLVKVKAAGICGSDLHIYKGGLGLMYPGPGDGTHLFPVKLPLVLGHEGAGVVEEVGSGVTGFKLKVGKFKVGDRVVVLPLVGCCGRGSAECEFCKGSGRENLCPKGRATGPGKGLMPNDGFGGFTPKKQGAPCKGKDGYHFMGDGGFAEYVVVPARRNDYFVVKIPDGLDDEIPLEEAEAAALLGCAGLTAYGALVRAAKVGSLPPGDTVLVHGAGGGVGLAAVQLAKAAGAARVIAVDSSEDPEKKLELAKELGADLDADFVNNSKGLPTVNDDRKEDFVEAIKELTGGRNGAGGVDVVLDCVGIGLGGATLDAALALLKPGGRLVVVGPKVAVGVPGGGAPIPLLLLKEEEKLYERSIKGSFLGGRKPRLSVLSVDTTPDELREALDLLASGIKDKNGKGVLDPLITHTLPPLDDSLEEANEAFELLESGKHGKVVLIP

SEQ ID NO: 47 - protein, 151 aa, Artificial Sequence
  consensus sequence
ALISLALNLLLMLIKLIGGVLSGSLALLADALHSLSDVASSLISLIALRLAEKPPDEKHPFGHHRAETLAALLNSVFLVIVSFLEILYEAIERLISPDYEIPPDAVLAADIMEPEEPGLFEVGGVALGVALGGTALVVLLGLVVNLALHGY

SEQ ID NO: 48 - protein, 166 aa, Artificial Sequence
  consensus sequence
KKLKSEHNLNVRAAALHVLGDALSSVGVLIAALLIYFTGYSFKGWKWWYYADPIASILISLIILYTAFRLLKESVLILLEGTPSKEDLERKIKKTLLSIPGVKGVHDLHIWYLGSNKFIASVHVEVDDNLDLKEAHDILAEIEREILHKFGIEHVTVHVEPASEEE

SEQ ID NO: 49 - protein, 507 aa, Rattus norvegicus
MGCWGRNRGRLLCMLLLTFMFMVLEVVVSRVTASLAMLSDSFHMLSDVLALVVALVAERFARRTHATQKNTFGWIRAEVMGALVNAIFLTGLCFAILLEAVERFIEPHEMQQPLVVLSVGVAGLLVNVLGLCLFHHHSGEGQGAGHGHSHGHGHGHLAKGARKAGRAGGEAGAPPGRAPDQEPDQEETNTLVANTSNSNGLKADQAEPEKLRSDDPVDVQVNGNLIQESDSLESEDNRAGQLNMRGVFLHVLGDALGSVIVVVNALVFYFSWKGCTEDDFCVNPCFPDPCKSSVELMNSTQAPMHEAGPCWVLYLDPTLCIIMVCILLYTTYPLLKESALILLQTVPKQIDIKHLVKELRDVEGVEEVHELHVWQLAGSRIIATAHIKCEDPASYMQVAKTIKDVFHNHGIHATTIQPEFASVGSKSSVVPCELACRTQCALKQCCGTRPQVHSGKEAEKAPTVSISCLELSENLEKKPRRTKAEGSVPAVVIEIKNVPNKQPESSL

SEQ ID NO: 50 - protein, 240 aa, Homo sapiens
MGDALGSVVVVITAIIFYVLPLKSEDPCNWQCYIDPSLTVLMVIIILSSAFPLIKETAAILLQMVPKGVNMEELMSKLSAVPGISSVHEVHIWELVSGKIIATLHIKYPKDRGYQDASTKIREIFHHAGIHNVTIQFENVDLKEPLEQKDLLLLCNSPCISKGCAKQLCCPPGALPLAHVNGCAEHNGGPSLDTYGSDGLSRRDAREVAIEVSLDSCLSDHGQSLNKTQEDQCYVNRTHF

SEQ ID NO: 51 - protein, 322 aa, Artificial Sequence
  consensus sequence
ALISLALNLLLMLIKLIGGVLSGSLALLADALHSLSDVASSLISLIALRLAEKPPDEKHPFGHHRAETLAALLNSVFLVIVSFLEILYEAIERLISPDYEIPPDAVLAADIMEPEEPGLFEVGGVALGVALGGTALVVLLGLVVNLALHGYLRRVGKKLKSEHNLNVRAAALHVLGDALSSVGVLIAALLIYFTGYSFKGWKWWYYADPIASILISLIILYTAFRLLKESVLILLEGTPSKEDLERKIKKTLLSIPGVKGVHDLHIWYLGSNKFIASVHVEVDDNLDLKEAHDILAEIEREILHKFGIEHVTVHVEPASEEE

SEQ ID NO: 52 - protein, 388 aa, Homo sapiens
MEPSPAAGGLETTRLVSPRDRGGAGGSLRLKSLFTEPSEPLPEESKPVEMPFHHCHRDPLPPPGLTPERLHARRQLYAACAVCFVFMAGEVVGGYLAHSLAIMTDAAHLLADVGSMMGSLFSLWLSTRPATRTMTFGWHRSETLGALASVVSLWMVTGILLYLAFVRLLHSDYHIEGGAMLLTASIAVCANLLMAFVLHQAGPPHSHGSRGAEYAPLEEGPEQPLPLGNTSVRAAFVHVLGDLLQSFGVLAASILIYFKPQYKAADPISTFLFSICALGSTAPTLRDVLRILMEGTPRNVGFEPVRDTLLSVPGVRATHELHLWALTLTYHVASAHLAIDSTADPEAVLAEASSRLYSRFGFSSCTLQVEQYQPEMAQCLRCQEPPQA

SEQ ID NO: 53 - protein, 125 aa, Artificial Sequence
  consensus sequence
SGSLALLADALHSLSDVASSLISLIALRLAEKPPDEKHPFGHHRAETLAALLNSVFLVIVSFLEILYEAIERLISPDYEIPPDAVLAADIMEPEEPGLFEVGGVALGVALGGTALVVLLGLVVNL

SEQ ID NO: 54 - protein, 96 aa, Artificial Sequence
  consensus sequence
ADPIASILISLIILYTAFRLLKESVLILLEGTPSKEDLERKIKKTLLSIPGVKGVHDLHIWYLGSNKFIASVHVEVDDNLDLKEAHDILAEIEREI

SEQ ID NO: 55 - protein, 492 aa, Mus musculus
MPFTTVIFFVMILDFYMDSVCSVKMDVSKCARYGSFPIFISALLFGNFWTHPITDQLRAMNRAAHQESTEHVLSGGVVVSAVFFILSANIPSSPSKRGQKGTLIGYSPEGTPLYHFMGDAFQHSSQSVPRFIKDSLKQVLEESDSRQIFYFLCLNLLFTFVELFYGALTNSLGLISDGFHMLFDCSALVMGLFAALMSRWKATRIFSYGYGRIEILSGFINGLFLIVIAFFVFMESVARLIDPPELDTNMLTPVSVGGLIVNLIGICAFSHAHSHGHGASQGNCHSDHGHSHHAHGHGHDHGHSHGFSGGGMNANMRGVFLHVLADTLGSIGVIVSTVLIEQFGWFIADPLCSLFIAVLIFLSVIPLIKDACQVLLLRLPPDHEKELHIALEKIQKIEGLISYRDPHFWRHSASIVAGTIHIQVTSEVLEQRIVQQVTGILKDAGVNNLTIQVEKEAYFQHMSGLSTGFHDVLAMTKQMESLKYCKDGTYIM

SEQ ID NO: 56 - protein, 208 aa, Homo sapiens [sequence omitted]

SEQ ID NO: 57 - protein, 322 aa, Artificial Sequence; identical to SEQ ID NO: 51
  consensus sequence

SEQ ID NO: 58 - protein, 289 aa, Artificial Sequence
  consensus sequence
ALISLALNLLLMLIKLIGGVLSGSLALLADALHSLSDVASSLISLIALRLAEKPPDEKHPFGHHRAETLAALLNSVFLVIVSFLEILYEAIERLISPDYEIPPDAVLAADIMEPEEPGLFEVGGVALGVALGGTALVVLLGLVVNLALHGYLRRVGKKLKSEHNLNVRAAALHVLGDALSSVGVLIAALLIYFTGYSFKGWKWWYYADPIASILISLIILYTAFRLLKESVLILLEGTPSKEDLERKIKKTLLSIPGVKGVHDLHIWYLGSNKFIASVHVEVDDNLDLK

SEQ ID NO: 59 - protein, 378 aa, Mus musculus
MLPLSIKDDEYKPPKFNLFGKISGWFRSILSDKTSRNLFFFLCLNLSFAFVELLYGIWSNCLGLISDSFHMFFDSTAILAGLAASVISKWRDNDAFSYGYVRAEVLAGFVNGLFLIFTAFFIFSEGVERALAPPDVHHERLLLVSILGFVVNLVGIFVFNHGGHGHSHGSGHGHSHSLFNGALDHSHGHEDHCHSHEAKHGAAHSHDHDHAHGHGHLHSHDGPSFKATAGPSRQILQGVFLHILADTLGSIGVIASAIMMQNFGLMIADPICSILIAILIVVSVIPLLRESVGILMQRTPPSLENTLPQCYQRVQQLQGVYNLQEQHFWTLCSDVYVGTLKLVVAPDADARWILSQTHNIFTQAGVRQLYVQIDFAAM

SEQ ID NO: 60 - protein, 54 aa, Artificial Sequence
  exemplary motif
HXXXXXXXXXXXXXCXXCXXXXXXXXXXXXXXCXXCXXXXXXXCXXXXXXXXXC

SEQ ID NO: 61 - protein, 9 aa, Artificial Sequence
  exemplary motif
XXDEADXXX

SEQ ID NO: 62 - protein, 25 aa, Artificial Sequence
  exemplary motif
CXXCXXXXXXXXXXXXXXXXXCXXC

SEQ ID NO: 63 - DNA, 3624 bp, Homo sapiens
  CDS (339)...(1361)
tagtctaactcgcggctgtcaccgccactgcagcggagccggccggccgggcgctgcgggacgggcgggcggctgccggcaggaggcgccgagccgggtgactgccgcggcgggcacagtccggggccacagcgccgagcccgggcgggagtggccccgcgcaggcagggagcggcgccgcgcactccaacccggcgggcacctcgggggcgggcgcggggcgcagccttctcgtcccggcctctgtgacaagcgccccggagccgggagcccgattgccgggctcggggtgggcgcggacgcaggcactgggctcgtgcggggccccgggcgtcgcgatgaacatcgtggtggagttcttcgtggtcactttcaaagtgctctgggcgttcgtgctggccgcggcgcgctggctggtgcggcccaaggagaagagcgtggcgggccaggtgtgcctcatcaccggcgccggcagcggcctgggccgcctcttcgcgctggagttcgcccggcgtcgggcgctgctggtgctgtgggacatcaacacgcaaagcaacgaggagacggctggcatggtgcgccacatctaccgcgacctggaggcggccgacgccgctgcgctgcaagctgggaatggtgaggaagaaattctgccccactgtaacttgcaggtttttacctacacctgtgacgtggggaagagggagaacgtctacctgacggctgaaagagtccgcaaggaggttggcgaagtctcagtcctggtcaataatgctggtgtggtctctgggcatcaccttctggaatgtcctgatgagctcattgagagaaccatgatggtcaattgccatgcacacttctggaccactaaggcttttcttcctacgatgctggagattaatcatggtcatattgtgacagttgcaagttccttgggattgttcagtactgccggagttgaggattactgtgccagtaaatttggagttgtgggttttcatgaatccctgagccatgaactaaaggctgctgaaaaggatggaattaaaacaaccttggtttgcccttatcttgtagacactggcatgttcagaggctgccgaatcaggaaagaaattgagccttttctgccacctctgaagcctgattactgtgtgaagcaggccatgaaggccatcctcactgaccagcccatgatctgcactccccgcctcatgtacatcgtgaccttcatgaagagcatcctaccatttgaagcagttgtgtgcatgtatcggttcctaggagcggacaagtgtatgtacccctttattgctcaaagaaagcaagccacaaacaataatgaagcaaaaaatggaatctaagaatctttttgtatggaatattacttctatcagaagatgatcaagatgtttcagtccagtgcacatcagcattgctgacattttatggattctaaacttgtgttgtttcttttttaaatcaactttttaaaaaaataaagtgtaaattaaccgactagagtacttggaaaatgtgatcagtacaagtgaacttaggttgttgccaacagggtccttttaggcagaacccagaaaccagtcaaatctgtagagaagcagtgtgacatcttcaggttaccattattttttaatgagcaggaagtctagaaatgataactagactgtatgtttcatgtgtgtgatttttcagaattcccagagtttactcattcttgttattaaactctagccagttgacatcttcgcaatttcaaggactgatagtgctgtattttctcacgttttctaagtttccgttttgcaaggcctaggtgactttttcatggtgtttgtatgtttagctcttttgaaaaggaattttgaaatctccatcaactgaagtaaatgatgtctgagtgttacagtwaaggtgaccaagtctctttcttaaagtcacaatgactaaagtattagttgaatttttttttttttttttgatggagtctcgctctgtcaccaggctggagtgcagtagcacaatcacggctcactgcaatctctgcctcccrgtttcaagtgattctgctgtctcagcctcccaagtagctgggactacaggcatgcgccaccacgcccagctaatttttgtatttttagtagagacggggtttcaccatgttggtcaggatggtctccatctcttgacattgtgatccacctgcctcggcctcccaaagtgctgggattacaggcatgagccactgcacccagccttgaatttttaattttatctctgatatacttcattaagtgtctggagacctaattatcctaaaagatcatacattttctacctatgaattttgctgcatacagaaagtgccctttcctcaggaagttgctgtgtttcatttctttggatggactcttatctagaatacatagcagctctgcaaagaaacagtttttaaaaatgggaacttctacattgaaaagtccccatttttgtgccaactatgattagtgagaggaagaaatcttattctatggcatatgtatggaagggtgtaaagattcttttgaaaggtttattcacattgtagaacagcaaatgacatttttacagtatttttttgtaaagcaaactattttgtgccttgaatttggtatatgtgtattagtgaaacattgtaaaggtgaacttctacctctgtatctaaatgtataccatccacttgtaaatgactataaactattatgtgattgcttttttttttagaatgtcttgtttaaatagtggccaatgtttaaggctgttaaaataagccaacttttactaattggggagttttataaatgactgattaaatttaaagaattaacttacatgcaattgtgtgattattagttatcagcagtgttgtaaggaaaattattgtgtttttttttatgatcattatcccactttaggtaaagaaaaatattggaatggaatagtgttgggaaacagacattaacaacctagggtgcctgcactcaaatagccgatgttactgtccctagattagagacttgattaagggcttgtttgtaccaaaagtggggaaacaatgccatgacctgtgttttagtttggctgcaccacagatcaaatctgcactgtgtctacatataggaaaggtcctggtgtgtgctaatgttcccaatgcaggacttgaggaagagctctgttatatgtttccatttctctttatcaaagataaccaaaccttatggcccttataacaatggaggcactggctgcctcttaattttcaatcatggacctaaagaagtactctgaagggtctcaacaatgccaggtggggacagatatactcagagattatccaggtctgcctcccagcgagcctggagtacaccagaccctcctagagaaatctgttataatttaacaacccacttatccaccttaaaactgaggaaagtcgtctttacatctaattttattcttgtgtgttataacttaaacctatttctatttttgtttgttattgcccttataagggtgtccatctccaagttcaataaactaattcatttaaaaaaaaaaaaaaaaaaa

SEQ ID NO: 64 - protein, 341 aa, Homo sapiens
MNIVVEFFVVTFKVLWAFVLAAARWLVRPKEKSVAGQVCLITGAGSGLGRLFALEFARRRALLVLWDINTQSNEETAGMVRHIYRDLEAADAAALQAGNGEEEILPHCNLQVFTYTCDVGKRENVYLTAERVRKEVGEVSVLVNNAGVVSGHHLLECPDELIERTMMVNCHAHFWTTKAFLPTMLEINHGHIVTVASSLGLFSTAGVEDYCASKFGVVGFHESLSHELKAAEKDGIKTTLVCPYLVDTGMFRGCRIRKEIEPFLPPLKPDYCVKQAMKAILTDQPMICTPRLMYIVTFMKSILPFEAVVCMYRFLGADKCMYPFIAQRKQATNNNEAKNGI

SEQ ID NO: 65 - DNA, 1026 bp, Homo sapiens
atgaacatcgtggtggagttcttcgtggtcactttcaaagtgctctgggcgttcgtgctggccgcggcgcgctggctggtgcggcccaaggagaagagcgtggcgggccaggtgtgcctcatcaccggcgccggcagcggcctgggccgcctcttcgcgctggagttcgcccggcgtcgggcgctgctggtgctgtgggacatcaacacgcaaagcaacgaggagacggctggcatggtgcgccacatctaccgcgacctggaggcggccgacgccgctgcgctgcaagctgggaatggtgaggaagaaattctgccccactgtaacttgcaggtttttacctacacctgtgacgtggggaagagggagaacgtctacctgacggctgaaagagtccgcaaggaggttggcgaagtctcagtcctggtcaataatgctggtgtggtctctgggcatcaccttctggaatgtcctgatgagctcattgagagaaccatgatggtcaattgccatgcacacttctggaccactaaggcttttcttcctacgatgctggagattaatcatggtcatattgtgacagttgcaagttccttgggattgttcagtactgccggagttgaggattactgtgccagtaaatttggagttgtgggttttcatgaatccctgagccatgaactaaaggctgctgaaaaggatggaattaaaacaaccttggtttgcccttatcttgtagacactggcatgttcagaggctgccgaatcaggaaagaaattgagccttttctgccacctctgaagcctgattactgtgtgaagcaggccatgaaggccatcctcactgaccagcccatgatctgcactccccgcctcatgtacatcgtgaccttcatgaagagcatcctaccatttgaagcagttgtgtgcatgtatcggttcctaggagcggacaagtgtatgtacccctttattgctcaaagaaagcaagccacaaacaataatgaagcaaaaaatggaatctaa

SEQ ID NO: 66 - DNA, 1327 bp, Homo sapiens
  CDS (367)...(1188)
cctgctgcaatggcttacgggagccaatgtgacgggatcagggcagacccatttagggtttcgtaaccggccaattcagtacgcaatagggaaaatcaattaggatctgcagagggttcccggatacaccttgcgaagaatgccgcactctccgccactcattccccactcaccggcacccgctaaaccttcagcctgaaattttcctccgaaggaagcagagcagaggaagaactaccaagtgctacactcaaagcctgccgtcgcagtgagcgcgacctccaaactgaggcatttttgttccggcgaaatccctcccactcaggaaagtccctagaaagagagcgcaggcgcctggggtatcacatgaccacttcccggaagcgcagcagacccgctcaacttcatcctgggttgaggcggaggagaacttccagaattatggcgaagtccgggctgaggcaggacccgcagagcacagctgcagccactgtgctaaagcgggcagtagaactagattcggagtcgcggtatccgcaggctctggtgtgttaccaagaggggattgatctgctcctgcaggttctgaaaggtaccaaagataatactaagagatgtaatctcagagaaaaaatttccaaatacatggacagagcggaaaacataaagaagtacttggaccaagaaaaagaagatggaaaatatcacaagcaaattaaaatagaagagaatgcaacaggtttcagttatgagtcactttttcgcgaataccttaatgagacagttacagaagtttggatagaagatccttatattagacatactcatcagctgtataactttcttcgattttgtgagatgcttattaagagaccatgtaaagtaaaaactattcaccttctcacctctctggatgaaggcattgagcaagtgcagcaaagtagaggcctgcaagaaatagaagagtcactcaggagtcacggagtgctgttggaagttcaatactcttcttcaatacatgaccgagaaattaggttcaacaatggatggatgattaagattggaaggggacttgattattttaagaaaccacagagtcgtttttcccttggatattgtgattttgatttaagaccatgtcatgaaacaacagtagacatttttcataagaagcatacaaaaaatatatgatgggtggtagcctaatttgtattatgtctactttaagtgaatattggattttttttaaaagatcacttttataatgtatgaatttaacaataaacttttatatttctactaaaaaaaaaaaaaaaaaaaaaaaaaa

SEQ ID NO: 67 - protein, 274 aa, Homo sapiens
MTTSRKRSRPAQLHPGLRRRRTSRIMAKSGLRQDPQSTAAATVLKRAVELDSESRYPQALVCYQEGIDLLLQVLKGTKDNTKRCNLREKISKYMDRAENIKKYLDQEKEDGKYHKQIKIEENATGFSYESLFREYLNETVTEVWIEDPYIRHTHQLYNFLRFCEMLIKRPCKVKTIHLLTSLDEGIEQVQQSRGLQEIEESLRSHGVLLEVQYSSSIHDREIRFNNGWMIKIGRGLDYFKKPQSRFSLGYCDFDLRPCHETTVDIFHKKHTKNI

SEQ ID NO: 68 - DNA, 825 bp, Homo sapiens
atgaccacttcccggaagcgcagcagacccgctcaacttcatcctgggttgaggcggaggagaacttccagaattatggcgaagtccgggctgaggcaggacccgcagagcacagctgcagccactgtgctaaagcgggcagtagaactagattcggagtcgcggtatccgcaggctctggtgtgttaccaagaggggattgatctgctcctgcaggttctgaaaggtaccaaagataatactaagagatgtaatctcagagaaaaaatttccaaatacatggacagagcggaaaacataaagaagtacttggaccaagaaaaagaagatggaaaatatcacaagcaaattaaaatagaagagaatgcaacaggtttcagttatgagtcactttttcgcgaataccttaatgagacagttacagaagtttggatagaagatccttatattagacatactcatcagctgtataactttcttcgattttgtgagatgcttattaagagaccatgtaaagtaaaaactattcaccttctcacctctctggatgaaggcattgagcaagtgcagcaaagtagaggcctgcaagaaatagaagagtcactcaggagtcacggagtgctgttggaagttcaatactcttcttcaatacatgaccgagaaattaggttcaacaatggatggatgattaagattggaaggggacttgattattttaagaaaccacagagtcgtttttcccttggatattgtgattttgatttaagaccatgtcatgaaacaacagtagacatttttcataagaagcatacaaaaaatatatga

SEQ ID NO: 69 - protein, 206 aa, Artificial Sequence
  consensus sequence
KVALVTGASSGIGLAIAKRLAKEGAKVVVADRNEEKLEKGAVAKELKELGGNDKDRALAIQLDVTDEESVKAAVEQAVERLGRGLDVLVNNAGGIILLRPGPFAELSRTMEEDWDRVIDVNLTGVFLLTRAVLPLMAMKKRGGGRIVNISSVAGRKEGGLVGVPGGSAYSASKAAVIGLTRSLALELAPHGGIRVNAVAPGGVDTD

SEQ ID NO: 70 - protein, 34 aa, Artificial Sequence
  consensus sequence
AEAYYNLGNAYLKLGKYDEAIEDYEKALELDPNN

SEQ ID NO: 71 - protein, 228 aa, Artificial Sequence
  consensus sequence
AKELLIRAVECDQVGRILEAQTLYTEGIGQLMQFVNGEPDEAKRKGFLTRIKEYMDRADAIKARINGKLMLGEVVSHVSIDENDTGFDYDQLFGKYMDDKTVEIMLEEPYMTQNYQYQNLIRFLELAATNCPNLKYFRLITKEYKDAKNPDQQETNLGQIKGDLERRNVTVYIKYEDSLHDRKIYLSNGYIIKIGRGLHFYKPANPMYSIGLVNYKFRKCLQTDVDIW

SEQ ID NO: 72 - protein, 59 aa, Artificial Sequence
  consensus sequence
AIDLVQKAIDEDKAGQYEEAYQLYQHALDYFMHALKYEAKNDKSKEIIRAKCTEYLDRA

SEQ ID NO: 73 - DNA, 1603 bp, Homo sapiens
  CDS (272)...(1603)
ttatggttttgtagaatttctttgacgaaccattggcaaaaagcaggagcaatccattctctggcgtgaattccacagtccatccaaatgattttcttgggattaccagatggttggctgatcttcagataatacatggggtgggtctcctaggtcactcctaggaaatgctgtgtcaccacttccttgtacttctcactgatctctctcatccactcatagatctctcccatggggtggtatatgttataggaatacgtctccaggctccatggactcactgacttgtccacaatctcttgtctgtgttgggctaaggatcagttggctttttgaaaaaagtgttttctctttcactgcttgctcaccctggccaacatgtggtaggcgcagggtcctcagcctttccgtgaagacgatcaacgttgctctttcccacgtgctgatgtattttctacctgcccgggcggccgctcgagcggctactcactatagggctcgagcggctactcactatagggctcgagcggccactcactatagggctcgagcggctactcactatagggctcgagcggccgcccgggcaggtattcattctcaaggacacttgatccactgccagagaggcccagaattttctaacttactgtgtggcagaatgaagcctctgcttgaaaccctttatcttttggggatgctggttcctggagggctgggatatgatagatccttagcccaacacagacaagagattgtggacaagtcagtgagtccatggagcctggagacgtattcctataacatataccaccccatgggagagatctatgagtggatgagagagatcagtgagaagtacaaggaagtggtgacacagcatttcctaggagtgacctatgagacccaccccatatattatctgaagatcagccaaccatctggtaatcccaagaaaatcatttggatggactgtggaattcacgccagagaatggattgctcctgctttttgccaatggttcgtcaaagaaattctacaaaaccataaagacaactcaaggatacgcaagctccttaggaacctggacttctatgtccttccagttcttaacatagatggttatatctacacttggacaactgatcgtctttggaggaaatcccgttcaccccataataatggcacatgttttgggacggatctcaatcgaaatttcaatgcatcttggtgtagtattggtgcctctagaaactgccaagatcaaacattctgtgggacagggccagtgtctgaaccagagactaaagctgttgccagcttcatagagagcaagaaggatgatattttgtgcttcctgaccatgcactcttatgggcagttaattctcacaccttacggctacaccaaaaataaatcaagtaaccacccagaaatgattcaagttggacagaaggcagcaaatgcattgaaagcaaagtatggaaccaattatagagttggatcgagtgcagatattttatatgcctcatcagggtcttcaagagattgggcccgagacattgggattcccttctcatatacgtttgagctgagggacaga

SEQ ID NO: 74 - protein, 444 aa, Homo sapiens [sequence omitted]

SEQ ID NO: 75 - DNA, 1332 bp, Homo sapiens
atggactcactgacttgtccacaatctcttgtctgtgttgggctaaggatcagttggctttttgaaaaaagtgttttctctttcactgcttgctcaccctggccaacatgtggtaggcgcagggtcctcagcctttccgtgaagacgatcaacgttgctctttcccacgtgctgatgtattttctacctgcccgggcggccgctcgagcggctactcactatagggctcgagcggctactcactatagggctcgagcggccactcactatagggctcgagcggctactcactatagggctcgagcggccgcccgggcaggtattcattctcaaggacacttgatccactgccagagaggcccagaattttctaacttactgtgtggcagaatgaagcctctgcttgaaaccctttatcttttggggatgctggttcctggagggctgggatatgatagatccttagcccaacacagacaagagattgtggacaagtcagtgagtccatggagcctggagacgtattcctataacatataccaccccatgggagagatctatgagtggatgagagagatcagtgagaagtacaaggaagtggtgacacagcatttcctaggagtgacctatgagacccaccccatatattatctgaagatcagccaaccatctggtaatcccaagaaaatcatttggatggactgtggaattcacgccagagaatggattgctcctgctttttgccaatggttcgtcaaagaaattctacaaaaccataaagacaactcaaggatacgcaagctccttaggaacctggacttctatgtccttccagttcttaacatagatggttatatctacacttggacaactgatcgtctttggaggaaatcccgttcaccccataataatggcacatgttttgggacggatctcaatcgaaatttcaatgcatcttggtgtagtattggtgcctctagaaactgccaagatcaaacattctgtgggacagggccagtgtctgaaccagagactaaagctgttgccagcttcatagagagcaagaaggatgatattttgtgcttcctgaccatgcactcttatgggcagttaattctcacaccttacggctacaccaaaaataaatcaagtaaccacccagaaatgattcaagttggacagaaggcagcaaatgcattgaaagcaaagtatggaaccaattatagagttggatcgagtgcagatattttatatgcctcatcagggtcttcaagagattgggcccgagacattgggattcccttctcatatacgtttgagctgagggacaga

SEQ ID NO: 76 - DNA, 1748 bp, Homo sapiens
  CDS (197)...(1081)
gccgagtgctaggcacccgggctcttctgggggctccagaggcgccgcccaagagaccctgggccggcgccgggcgcagctgcctctccgtctttgtgtctgtctctgtgtctgtctggctatctccgagtttgcctccgcttccagaactaagccacccagacaccatcatcccgaaaaccccagcccttctcccatggcaggctacttgccccccaaaggctacgccccttcgcccccacctccctaccctgtcacccctgggtacccggagccggcgctacatcctgggcccgggcaggcgccagtgcccgcccaggtacctgccccagctcccggcttcgccctcttcccctcgcctggccccgtggccttggggtctgctgcccccttcttgccactgccaggggtgccttctggcctcgaattcctggtgcagattgatcagattttgattcaccagaaggctgagcgagtggaaacgttcctaggctgggagacctgtaatcggtatgaactgcgctctggggccgggcagcccctgggtcaggcggccgaggagagcaactgctgcgcccgtctgtgctgtggcgcccgccggccgctgcgtgtccgcctggccgaccccggggaccgtgaggtgctgcgtttgctccgcccgctgcactgtggctgcagctgctgcccctgtggcctccaggagatggaagtacaggctccaccaggcaccaccattggccacgtgctacagacctggcatcccttcctccccaagttctccatccaggatgccgatcgccagacagtcttgcgagtggtggggccctgctggacctgtggctgtggcacagacaccaactttgaggtgaagactcgggatgaatcccgcagtgtgggccgcatcagcaagcagtgggggggcctggtccgagaagccctcacagatgcagatgactttggcctacagttcccgctggacctggatgtgagggtgaaggctgtgctgctgggagccacattcctcattgactacatgttctttgagaagcgaggaggcgctgggccctctgccatcaccagttagaggccaccatggtgtgaggagaccatcacctcgaccagaactccagatggtcacctgccctggcccctcctctgggcagcccctttcctccatgtacactgcaggggacagaaggggggccccatccctaccctactccctggccgcctgcccctgtggttcccaaggaggggtatgtatgagagccgctctcctgctacctcccaccactgtcccagcagtccctcggcacacaggcatatcagctttcacactttccccatgcactctctcccacccccttccagggcctctgctccaaaggaggcctctggaacccaggactctggggttttacaagagggctggggtgtggaagggcaagctgcaccaaagacggtggatatagccaccgcccccccgccgctgcctagcatctgcttggccaattagttcagcctcagaccatggcactttgagggggtctctacctccccatcaacagctgcagggggaccccagtgccaacttcctctcccactagggccctgccttcagctggtgcttgctgcgattcctgtgccttatgtaactgcccttccttcccttgccctaggaaaaaggctgcatctttatatgttacattcatataaactttgtaactttttggaaaaaaaaaaaaaaa

SEQ ID NO: 77 - protein, 295 aa, Homo sapiens
MAGYLPPKGYAPSPPPPYPVTPGYPEPALHPGPGQAPVPAQVPAPAPGFALFPSPGPVALGSAAPFLPLPGVPSGLEFLVQIDQILIHQKAERVETFLGWETCNRYELRSGAGQPLGQAAEESNCCARLCCGARRPLRVRLADPGDREVLRLLRPLHCGCSCCPCGLQEMEVQAPPGTTIGHVLQTWHPFLPKFSIQDADRQTVLRVVGPCWTCGCGTDTNFEVKTRDESRSVGRISKQWGGLVREALTDADDFGLQFPLDLDVRVKAVLLGATFLIDYMFFEKRGGAGPSAITS

SEQ ID NO: 78 - DNA, 888 bp, Homo sapiens
atggcaggctacttgccccccaaaggctacgccccttcgcccccacctccctaccctgtcacccctgggtacccggagccggcgctacatcctgggcccgggcaggcgccagtgcccgcccaggtacctgccccagctcccggcttcgccctcttcccctcgcctggccccgtggccttggggtctgctgcccccttcttgccactgccaggggtgccttctggcctcgaattcctggtgcagattgatcagattttgattcaccagaaggctgagcgagtggaaacgttcctaggctgggagacctgtaatcggtatgaactgcgctctggggccgggcagcccctgggtcaggcggccgaggagagcaactgctgcgcccgtctgtgctgtggcgcccgccggccgctgcgtgtccgcctggccgaccccggggaccgtgaggtgctgcgtttgctccgcccgctgcactgtggctgcagctgctgcccctgtggcctccaggagatggaagtacaggctccaccaggcaccaccattggccacgtgctacagacctggcatcccttcctccccaagttctccatccaggatgccgatcgccagacagtcttgcgagtggtggggccctgctggacctgtggctgtggcacagacaccaactttgaggtgaagactcgggatgaatcccgcagtgtgggccgcatcagcaagcagtgggggggcctggtccgagaagccctcacagatgcagatgactttggcctacagttcccgctggacctggatgtgagggtgaaggctgtgctgctgggagccacattcctcattgactacatgttctttgagaagcgaggaggcgctgggccctctgccatcaccagttag

SEQ ID NO: 79 - DNA, 2634 bp, Homo sapiens
  CDS (13)...(2631)
gcacgagcagccatggagtcgctcctgctgccggtgctgctgctgctggccatactgtggacgcaggctgccgccctcattaatctcaagtactcggtagaagaggagcagcgcgccgggacggtgattgccaacgtggccaaagacgcgcgagaggcgggcttcgcgctggacccccggcaggcttcagcctttcgcgtggtgtccaactcggctccacacctagtggacatcaatcccagctctggcctgctggtcaccaagcagaagattgaccgtgatctgctgtgccgccagagccccaagtgcatcatctcgctcgaggtcatgtccagctcaatggaaatctgcgtgataaaggtggagatcaaggacctgaacgacaatgcgcccagtttcccggcagcacggatcgagctggagatctcggaggcagccagccctggcacgcgcatcccgctggacagcgcttacgatccagactcaggaagctttggcgtgcagacttacgagctcacgcccaacgagctgttcggcctggagatcaagacgcgcggcgacggctcccgctttgccgaactcgtggtggaaaagagcctggaccgcgagacgcagtcgcactacagcttccgaatcactgcgctagacggtggcgacccgccgcgcctgggcaccgttggccttagtatcaaggtgaccgactccaatgacaacaacccggtgtttagcgagtccacctacgcggtgagcgtgccagaaaactcgcctcccaacacacccgtcatccgcctcaacgccagcgatccagacgagggcaccaacggccaggtggtctactccttctatggctacgtcaacgaccgcacgcgcgagctctttcagatcgacccgcacagtggcctggtcactgtcactggcgctttagactacgaagaggggcacgtgtacgaactggacgtgcaggctaaggacttggggcccaattccatcccggcacactgcaaggtcaccgtcagcgtgctggacaccaatgacaatccgccggtcatcaacctgctgtcagtcaacagtgagcttgtggaggtcagcgagagcgcccccccgggctacgtgatcgccttggtgcgggtgtctgatcgcgactcaggcctcaatggacgtgtgcagtgccgtttgctgggcaatgtgccctttcgactgcaggaatatgagagcttctccactattctggtggacggacggctggaccgcgagcagcacgaccaatacaacctcacaattcaggcacgcgacggcggcgtgcccatgctgcagagtgccaagtcctttaccgtgctcatcactgacgaaaatgacaaccacccgcacttttccaagccctactaccaggtcattgtgcaggagaacaacacgcctggcgcctatctgctctctgtgtctgctcgcgaccccgacctgggtctcaacggcagtgtctcctaccagatcgtgccgtcgcaggtgcgggacatgcctgtcttcacctatgtctccatcaatcccaactcaggcgacatctacgcgctgcgatcctttaaccacgagcagaccaaggcgttcgaattcaaggtgctggccaaggacggcggccttccctcactgcaaagcaacgctacggtgcgggtcatcatcctcgacgtcaacgacaacaccccggtcatcacagccccacctctgattaacggcactgccgaggtctacataccccgcaactctggcataggctacctggtgactgttgtcaaggcagaagactacgatgagggcgaaaatggccgagtcacctacgacatgaccgagggcgaccgcggcttctttgaaatagaccaggtcaatggcgaagtcagaaccacccgcaccttcggggagagctccaagtcctcctatgagcttatcgtggtggctcacgaccacggcaagacatctctctctgcctctgctctcgtcctaatctacttgtcccctgctctcgatgcccaagagtcaatgggctctgtgaacttgtccttgattttcattattgccctgggctccattgcgggcatcctctttgtaactatgatcttcgtggcaatcaagtgcaagcgagacaacaaagagatccggacctacaactgcagaattgctgagtactcctatgggcatcaaaagaaatcaagcaagaagaaaaaaatcagtaagaatgacaaccgcctggtaccccgggatgtggaggagacagacaagatgaacgttgtcagttgctcttccctgacctcctccctcaactattttgactaccaccagcagacgctgcccctgggctgccgccgctctgagagcactttcctgaatgtggagaaccagaatacccgcaacaccagtgctaaccacatctaccatcactctttcaacagccaggggccccagcagcctgacctgattatcaacggtgtgcctctgcctgagagcaaggaaagccagcagtggtactttgtcgctgctgcagcactctttcctcatgcctgttttgtgggctgctgtctgatatccactcctacagtgctttgggaacaatatcaagcagggcataaggctgtggcccaaagaaaatga

SEQ ID NO: 80 - protein, 873 aa, Homo sapiens
MESLLLPVLLLLAILWTQAAALINLKYSVEEEQRAGTVIANVAKDAREAGFALDPRQASAFRVVSNSAPHLVDINPSSGLLVTKQKIDRDLLCRQSPKCIISLEVMSSSMEICVIKVEIKDLNDNAPSFPAARIELEISEAASPGTRIPLDSAYDPDSGSFGVQTYELTPNELFGLEIKTRGDGSRFAELVVEKSLDRETQSHYSFRITALDGGDPPRLGTVGLSIKVTDSNDNNPVFSESTYAVSVPENSPPNTPVIRLNASDPDEGTNGQVVYSFYGYVNDRTRELFQIDPHSGLVTVTGALDYEEGHVYELDVQAKDLGPNSIPAHCKVTVSVLDTNDNPPVINLLSVNSELVEVSESAPPGYVIALVRVSDRDSGLNGRVQCRLLGNVPFRLQEYESFSTILVDGRLDREQHDQYNLTIQARDGGVPMLQSAKSFTVLITDENDNHPHFSKPYYQVIVQENNTPGAYLLSVSARDPDLGLNGSVSYQIVPSQVRDMPVFTYVSINPNSGDIYALRSFNHEQTKAFEFKVLAKDGGLPSLQSNATVRVIILDVNDNTPVITAPPLINGTAEVYIPRNSGIGYLVTVVKAEDYDEGENGRVTYDMTEGDRGFFEIDQVNGEVRTTRTFGESSKSSYELIVVAHDHGKTSLSASALVLIYLSPALDAQESMGSVNLSLIFIIALGSIAGILFVTMIFVAIKCKRDNKEIRTYNCRIAEYSYGHQKKSSKKKKISKNDNRLVPRDVEETDKMNVVSCSSLTSSLNYFDYHQQTLPLGCRRSESTFLNVENQNTRNTSANHIYHHSFNSQGPQQPDLIINGVPLPESKESQQWYFVAAAALFPHACFVGCCLISTPTVLWEQYQAGHKAVAQRK

SEQ ID NO: 81 - DNA, 2622 bp, Homo sapiens
atggagtcgctcctgctgccggtgctgctgctgctggccatactgtggacgcaggctgccgccctcattaatctcaagtactcggtagaagaggagcagcgcgccgggacggtgattgccaacgtggccaaagacgcgcgagaggcgggcttcgcgctggacccccggcaggcttcagcctttcgcgtggtgtccaactcggctccacacctagtggacatcaatcccagctctggcctgctggtcaccaagcagaagattgaccgtgatctgctgtgccgccagagccccaagtgcatcatctcgctcgaggtcatgtccagctcaatggaaatctgcgtgataaaggtggagatcaaggacctgaacgacaatgcgcccagtttcccggcagcacggatcgagctggagatctcggaggcagccagccctggcacgcgcatcccgctggacagcgcttacgatccagactcaggaagctttggcgtgcagacttacgagctcacgcccaacgagctgttcggcctggagatcaagacgcgcggcgacggctcccgctttgccgaactcgtggtggaaaagagcctggaccgcgagacgcagtcgcactacagcttccgaatcactgcgctagacggtggcgacccgccgcgcctgggcaccgttggccttagtatcaaggtgaccgactccaatgacaacaacccggtgtttagcgagtccacctacgcggtgagcgtgccagaaaactcgcctcccaacacacccgtcatccgcctcaacgccagcgatccagacgagggcaccaacggccaggtggtctactccttctatggctacgtcaacgaccgcacgcgcgagctctttcagatcgacccgcacagtggcctggtcactgtcactggcgctttagactacgaagaggggcacgtgtacgaactggacgtgcaggctaaggacttggggcccaattccatcccggcacactgcaaggtcaccgtcagcgtgctggacaccaatgacaatccgccggtcatcaacctgctgtcagtcaacagtgagcttgtggaggtcagcgagagcgcccccccgggctacgtgatcgccttggtgcgggtgtctgatcgcgactcaggcctcaatggacgtgtgcagtgccgtttgctgggcaatgtgccctttcgactgcaggaatatgagagcttctccactattctggtggacggacggctggaccgcgagcagcacgaccaatacaacctcacaattcaggcacgcgacggcggcgtgcccatgctgcagagtgccaagtcctttaccgtgctcatcactgacgaaaatgacaaccacccgcacttttccaagccctactaccaggtcattgtgcaggagaacaacacgcctggcgcctatctgctctctgtgtctgctcgcgaccccgacctgggtctcaacggcagtgtctcctaccagatcgtgccgtcgcaggtgcgggacatgcctgtcttcacctatgtctccatcaatcccaactcaggcgacatctacgcgctgcgatcctttaaccacgagcagaccaaggcgttcgaattcaaggtgctggccaaggacggcggccttccctcactgcaaagcaacgctacggtgcgggtcatcatcctcgacgtcaacgacaacaccccggtcatcacagccccacctctgattaacggcactgccgaggtctacataccccgcaactctggcataggctacctggtgactgttgtcaaggcagaagactacgatgagggcgaaaatggccgagtcacctacgacatgaccgagggcgaccgcggcttctttgaaatagaccaggtcaatggcgaagtcagaaccacccgcaccttcggggagagctccaagtcctcctatgagcttatcgtggtggctcacgaccacggcaagacatctctctctgcctctgctctcgtcctaatctacttgtcccctgctctcgatgcccaagagtcaatgggctctgtgaacttgtccttgattttcattattgccctgggctccattgcgggcatcctctttgtaactatgatcttcgtggcaatcaagtgcaagcgagacaacaaagagatccggacctacaactgcagaattgctgagtactcctatgggcatcaaaagaaatcaagcaagaagaaaaaaatcagtaagaatgacaaccgcctggtaccccgggatgtggaggagacagacaagatgaacgttgtcagttgctcttccctgacctcctccctcaactattttgactaccaccagcagacgctgcccctgggctgccgccgctctgagagcactttcctgaatgtggagaaccagaatacccgcaacaccagtgctaaccacatctaccatcactctttcaacagccaggggccccagcagcctgacctgattatcaacggtgtgcctctgcctgagagcaaggaaagccagcagtggtactttgtcgctgctgcagcactctttcctcatgcctgttttgtgggctgctgtctgatatccactcctacagtgctttgggaacaatatcaagcagggcataaggctgtggcccaaagaaaatga

SEQ ID NO: 82 - protein, 281 aa, Artificial Sequence
  consensus sequence
YHNLEEIYAWLDLLVSNFPDLVSKVSIGKSYEGRDLKVLKISDNPATGENEPEVFAVAGWIHAREWVTSATLLWLLKELVANYGSDKTITKLLDGLDLFYILPVFNPDGYAYSITTDSYRMWRKTRSPNAGSFCVGTDPNRNWYAQWGGMGASSYSPCSETYEGTAPFSEPETKAVEDFIRSWLGGGKQNIKAYITFHSYSQLLLYPYGYDYNLNPDANDLDELSDLKIAADALSARHGTYYTLGLPGSSTIYPASAGGSDDWAYDVGIIKYAFTFELRPD

SEQ ID NO: 83 - protein, 138 aa, Artificial Sequence
  consensus sequence
VMTCHRPRPCCSCCDPCCTQFIEVSAPPGQVIGIIEQECSCCNPKFNIMNSDGNTVLRIEGPCCTCSCCCSDTDFEVKTLDGEKVVGKISKQWGGLLREAFTDADNFGIHFPLDLDVKMKAVLLGACFLIDFMYFENR

SEQ ID NO: 84 - protein, 88 aa, Artificial Sequence
  consensus sequence
CTQFIEVSAPPGQVIGIIEQECSCCNPKFNIMNSDGNTVLRIEGPCCTCSCCCSDTDFEVKTLDGEKVVGKISKQWGGLLREAFTDAD

SEQ ID NO: 85 - protein, 107 aa, Artificial Sequence
  consensus sequence
YSASVPENAPVGTEVLTVTATDADDPLGPNGRIRYSILGGNPGGWFRIDPDTGDNEGIISTTKPLDREEIFNGEYELTVEATDADPLSAAGGSPPLSGTATVTITVL

SEQ ID NO: 86 - protein, 11 aa, Artificial Sequence
  consensus sequence
XXXXDXNDXXP